(19) United States
(12) Patent Application Publication
(10) Pub. No.: US 2006/0014166 A1
(43) Pub. Date: Jan. 19, 2006

(54) Novel nucleotide and amino acid sequences, and assays and methods of use thereof for diagnosis of endometriosis

(76) Inventors: Yossi Cohen, Banstead (GB); Sarah Pollock, Tel-Aviv (IL); Amit Novik, Beit-HaSharon (IL); Alexander Diber, Rishon-LeZion (IL)

Correspondence Address: STAAS & HALSEY LLP, SUITE 700, 1201 NEW YORK AVENUE, N.W., WASHINGTON, DC, 20005, US

(21) Appl. No.: 11/043,788

(22) Filed: Jan. 27, 2005

Related U.S. Application Data
(60) Provisional application No. 60/628,145, filed on Nov. 17, 2004.
(60) Provisional application No. 60/628,178, filed on Nov. 17, 2004.
(60) Provisional application No. 60/621,004, filed on Oct. 22, 2004.
(60) Provisional application No. 60/628,230, filed on Nov. 17, 2004.
(60) Provisional application No. 60/539,129, filed on Jan. 27, 2004.
(60) Provisional application No. 60/539,128, filed on Jan. 27, 2004.

(51) Int. Cl. C12Q 1/68; C07H 21/04; C12N 15/09; C07K 14/47; C07K 16/18

(52) U.S. Cl. 435/6; 435/69.1; 435/320.1; 435/325; 530/324; 530/388.25; 536/23.5

(57) ABSTRACT

Novel markers for endometriosis that are both sensitive and accurate. These markers are differentially expressed in endometriosis specifically, as opposed to normal tissue. The measurement of these markers, alone or in combination, in patient samples provides information that the diagnostician can correlate with a probable diagnosis of endometriosis. The markers of the present invention, alone or in combination, show a high degree of differential detection between endometriosis and non-endometriosis states.

### CROSS-REFERENCE TO RELATED APPLICATION(S)

THIS APPLICATION IS RELATED TO NOVEL NUCLEOTIDE AND AMINO ACID SEQUENCES, AND ASSAYS AND METHODS OF USE THEREOF FOR DIAGNOSIS OF ENDOMETRIOSIS, AND CLAIMS PRIORITY TO THE BELOW U.S. PROVISIONAL APPLICATIONS WHICH ARE INCORPORATED BY REFERENCE HEREIN:
APPLICATION No. 60/628,145 FILED Nov. 17, 2004—DIFFERENTIAL EXPRESSION OF MARKERS IN PANCREATIC CANCER II
APPLICATION No. 60/628,178 FILED Nov. 17, 2004—DIFFERENTIAL EXPRESSION OF MARKERS IN BRAIN CANCER II
APPLICATION No. 60/621,004 FILED Oct. 22, 2004—DIFFERENTIAL EXPRESSION OF MARKERS IN SKIN AND EPITHELIAL CANCER II
APPLICATION No. 60/628,230 FILED Nov. 17, 2004—DIFFERENTIAL EXPRESSION OF MARKERS IN ENDOMETRIOSIS
APPLICATION No. 60/539,129 FILED Jan. 27, 2004—METHODS AND SYSTEMS FOR ANNOTATING BIOMOLECULAR SEQUENCES
APPLICATION No. 60/539,128 FILED Jan. 27, 2004—EVOLUTIONARY CONSERVED SPLICED SEQUENCES AND METHODS AND SYSTEMS FOR IDENTIFYING THEREOF

### FIELD OF THE INVENTION

The present invention is related to novel nucleotide and protein sequences that are diagnostic markers for endometriosis, and assays and methods of use thereof.

### BACKGROUND OF THE INVENTION

Endometriosis represents one of the most common admitting diagnoses in women of reproductive age. It is defined as the presence of endometrial tissue outside of the uterus and is typically present in the pelvis such as on the ovaries and pelvic peritoneum. It may also involve the bowel, ureter or bladder. Endometriosis is a common gynecologic disorder that presents with chronic pelvic pain or infertility. The histologic diagnosis requires the presence of endometrial glands and stroma from a tissue sample. (Clin Chim Acta. 2004 February; 340(1-2):41-56). Endometriosis diagnosis is problematic. Studies in the USA, UK and Australia have demonstrated that the delay in the diagnosis of endometriosis is universal. For example, a study by the Australian Endometriosis Society in 1990 found a delay of approximately 4.4 years from consultation to diagnosis. Younger women are more likely to experience a delay in diagnosis. Those between 15-19 years of age experience an average delay to diagnosis of 8.3 years (Aust Fam Physician. 2001 July; 30(7):649-53).

The gold standard for the diagnosis of endometriosis is a surgical intervention, a laparoscopy. The severity of disease is variable and patients are usually categorized according to the American Fertility Society classification of disease into four groups that represent mild to severe disease, stages I to IV. There is a poor correlation between the severity of disease and the patient's symptoms. Furthermore, the disease can be found in asymptomatic patients. This heterogeneity in clinical presentation has contributed to the difficulties in identifying a marker. Since some women are asymptomatic, clinical trials require a control group of women that require a surgical procedure to exclude the presence of endometriosis. Considerable effort has been invested in searching for non-invasive methods of diagnosis (Clin Chim Acta. 2004 February; 340(1-2):41-56).

Serum CA-125, a 200,000 Da glycoprotein, concentration has been associated with the presence of many gynecologic disorders including endometriosis (Int J Biol Markers. 1998 October-December; 13(4):231-7). The CA-125 antigen is expressed in many normal tissues such as the endometrium, endocervix and peritoneum. In some women, CA-125 levels increase during menstruation. Mean CA-125 levels are higher during menses in patients with and without endometriosis and it is therefore recommended that CA-125 levels not be drawn during a menstrual period (Am J Obstet Gynecol. 1987 December; 157(6):1426-8). Many studies tried to assess the role of serum CA-125 measurement in the detection of endometriosis. The main confounding variable in determining the sensitivity and specificity of serum CA-125 is the stage of the disease. Typically, most patients with advanced endometriosis (and few patients with early stage disease) will have elevated serum CA-125 levels (similar to what occurs in ovarian cancer). A recent meta-analysis performed to assess the diagnostic performance of serum CA-125 in detecting endometriosis (Fertil Steril. 1998 December; 70(6):1101-8) Showed sensitivity ranged from 4% to 100% and the specificity ranged from 38% to 100% for the diagnosis of any stage of disease. The ROC curve showed a poor diagnostic performance. At a specificity of 90%, a sensitivity of 28% was reported. If the sensitivity was increased to 50%, the specificity dropped to 72%. For advanced disease, the sensitivity ranged from 0% to 100% and the specificity ranged from 44% to 95%. For a specificity of approximately 90%, the sensitivity was 47%. If the sensitivity was increased to 60%, the specificity dropped to 81% (Fertil Steril. 1998 December; 70(6):1101-8). According to the authors of this study, a negative result would delay the diagnosis in 70% of patients with endometriosis. The routine use of serum CA-125 cannot be advocated as a diagnostic tool to exclude the diagnosis of endometriosis in patients with chronic pelvic pain or infertility. CA-125 may be more useful in evaluating recurrent disease or the success of a surgical treatment. Many investigators have measured levels of CA-125 in the peritoneal fluid of patients with and without endometriosis (Gynecol Obstet Invest. 1990; 30(2):105-8). Although peritoneal fluid levels of CA-125 are almost 10 times higher than serum levels, no differences were found between women with and without Endometriosis (Fertil Steril. 1991 November; 56(5):863-9). CA-125 levels have also been measured in other body fluids such as menstrual discharge and uterine fluid but were not found to be useful in clinical practice.

CA 19-9 is a high-molecular-weight glycoprotein elevated in patients with malignant and benign ovarian tumors including ovarian chocolate cysts. Serum CA19-9 levels in women with endometriosis fell significantly after treatment for endometriosis when compared with the basal levels before treatment (Eur J Gynaecol Oncol. 1998; 19(5):498-500). There are a limited number of reports on the significance of serum CA19-9 levels in the diagnosis of endometriosis but the overall conclusion is that the clinical utility of the CA19-9 measurement is not superior to that of the CA-125. For example, in one study (Fertil Steril. 2002 October; 78(4):733-9) when comparing the sensitivities of the CA19-9 and CA-125 tests for the diagnosis of endometriosis, the authors found that the sensitivity of the CA19-9 test was significantly lower than that of the CA-125 test (34% and 49%, respectively).

Soluble forms of the intercellular-adhesion molecule-1 (sICAM-1) are secreted from the endometrium and endometriotic implants. Moreover, endometrium from women with endometriosis secretes a higher amount of this molecule than tissue from women without the disease. Consequently, a strong correlation exists between levels of sICAM-1 shed by the endometrium and the number of endometriotic implants in the pelvis (Obstet Gynecol. 2000 January; 95(1):115-8). It has been hypothesized that sICAM-1 may be useful in the diagnosis of endometriosis. A few studies reported a significant increase in serum concentration of sICAM-1 in patients with endometriosis (for example, Am J Reprod Immunol. 2000 March; 43(3):160-6) but overall it was shown that serum levels of sICAM-1 were only slightly but not significantly higher in women with endometriosis than in women without the disease unless the disease is of high stage (deep peritoneal) (Fertil Steril. 2002 May; 77(5):1028-31). The sensitivity and specificity of sICAM-1 in detecting deep peritoneal endometriosis were 19% and 97%, respectively. It has been shown that in women with deep infiltrating Endometriosis measurement of CA-125 and sICAM-1 together may improve diagnosis.

Serum placental protein 14 (PP-14)—currently known as glycodelin-A was found to be significantly higher in endometriosis patients than in healthy controls (Am J Obstet Gynecol. 1989 October; 161(4):866-71). Levels were significantly lowered by conservative surgery as well as by treatment with danazol and medroxy progesterone acetate. The ability of serum PP-14 levels to diagnose of endometriosis is limited because of a low sensitivity (59%). Typically, the peritoneal fluid concentrations of PP-14 are low. The levels are elevated in the luteal phase of endometriosis patients. It is controversial whether this is of any diagnostic importance or not.

Tumor necrosis factors (TNF) play an essential role in the inflammatory process. TNF is believed to involve in many physiological and pathological reproductive processes. The main TNF is TNF-a. In the human endometrium, TNF-a is a factor in the normal physiology of endometrial proliferation and shedding. TNF-a is expressed mostly in epithelial cells, particularly in the secretory phase. Stromal cells stain for TNF-a mostly in the proliferative phase of the menstrual cycle. Therefore it is believed it is probably influenced by hormones. TNF-a concentrations in peritoneal fluid are elevated in patients with endometriosis, but it is controversial whether they are correlated with disease stage or not (ertil Steril. 1988 October; 50(4):573-9). It has been suggested that measurement of TNF-a peritoneal fluid can be used as a foundation for non-surgical diagnosis of endometriosis but that hasn't been comprehensively checked (Hum Reprod. 2002 February; 17(2):426-31).

IL-6 is a regulator of inflammation and immunity and modulates secretion of other cytokines, promotes T-cell activation and B-cell differentiation and inhibits growth of various human cell lines. IL-6 is produced by different cells including endometrial epithelial stromal cells. The role of IL-6 in the pathogenesis of endometriosis has been extensively studied. IL-6 response is different in peritoneal macrophages, endometrial stromal cells and peripheral macrophages in patients with endometriosis (Fertil Steril. 1996 June; 65(6):1125-9). It has been shown that IL-6 was significantly elevated in the sera of endometriosis patients but not in their peritoneal fluid as compared with patients with unexplained infertility and tubal ligation/reanastomosis (Hum Reprod. 2002 February; 17(2):426-31). That finding was contradicted by other works but it is thought the different results might be attributed to the antibody specificity of the assay.

There has been some work on the proliferation and neovascularization of the endometriotic implants, and particularly on the role of Vascular endothelial growth factor (VEGF). The basic physiological function of VEGF is to induce angiogenesis, which allows the endometrium to repair itself following menstruation. It also modulates the characteristics of the newly formed vessels by controlling the microvascular permeability and permitting the formation of a fibrin matrix for endothelial cell migration and proliferation (Science 1985; 227:1059-61). This modulation may be responsible for local endometrial edema, which helps prepare the endometrium for embryo implantation. In endometriosis patients, VEGF is localized in the epithelium of endometriotic implants (J Clin Endocrinol Metab 1996; 81:3112-8), particularly in hemorrhagic red implants (Hum Reprod 1998; 13:1686-90). Moreover, the concentration of VEGF is increased in the peritoneal fluid of endometriosis patients. The exact cellular sources of VEGF in peritoneal fluid have not yet been precisely defined. Although evidence suggests that endometriotic lesions themselves produce this factor, activated peritoneal macrophages also can synthesize and secrete VEGF (Hum Reprod 1996; 11:220-3). Antiangiogenic drugs are potential therapeutic agents in endometriosis.

There are many more cytokines which were considered for the purpose of Endometriosis diagnosis, among them RANTES (Regulated on Activation, Normal T-Cell Expressed and Secreted) where in vitro secretion of RANTES by endometrioma-derived stromal cell cultures is significantly greater than in eutopic endometrium (Am J Obstet Gynecol 1993; 169:1545-9), IL-1 where research has shown that the administration of exogenous IL-1 receptor antagonist blocks successful implantation in mice (Endocrinology 1994; 134:521-8), IL-4, IL-5, IL-8, IL-10, IL-12, IL-13, interferon-gamma; MCP-1, MCSF and TGF. Most often, they have not been extensively investigated as a diagnostic tool. One group studies a panel of serum and peritoneal fluid such markers for the prediction of endometriosis (Hum Reprod. 2002 February; 17(2):426-31). Serum and peritoneal fluid from 130 women were obtained while they underwent laparoscopy for pain, infertility, tubal ligation or sterilization reversal. They measured the concentrations of 6 cytokines (IL-1, IL-6, IL-8, IL-12, IL-13 and TNF-a) in serum and peritoneal fluid and levels of reactive oxygen species (ROS) in peritoneal fluid. Only serum IL-6 and peritoneal fluid TNF-a could discriminate between patients with and without endometriosis with a high degree of sensitivity and specificity. The peritoneal fluid TNF-a had a very good 99% area under the curve but in that study all peritoneal fluid samples that were contaminated by blood (a common procedure artifact) were excluded from study. Therefore this result has only a partial practical value.

A few Endometrial tissue biochemical markers were investigated in the context of endometriosis. Aromatase P450 is a catalyst of the conversion of androstenedione and testosterone to estrone and estradiol, respectively. It is expressed in both eutopic and ectopic endometrium of endometriosis patients but not in eutopic endometrium of healthy controls (Biol Reprod 1997; 57:514-9). Although endometrial aromatase P450 expression does not correlate with the disease stage, a recent study demonstrated that detection of aromatase P450 transcripts in the endometrium of endometriosis patients may be a potential qualitative marker of endometriosis Fertil Steril 2002; 78:825-9). The potential use of such marker as a clinically useful diagnostic tool of pelvic disease is limited by the observation that large numbers of women with endometriosis do not express aromatase P450 in their eutopic endometrium. Cytokeratins 8, 18, 19, vimentin and human leukocyte class I antigens were shown to be immunoreactive in endometriosis cell lines (Hum Reprod Update 1997; 3:117-23). More genes have shown to be aberrantly regulated in the endometrium of women with endometriosis including avBeta3 integrin, beta1-integrin, E-cadherin, 17b-hydroxysteroid dehydrogenase type-1, Monocyte chemotactic protein-1, interleukin-1 receptor type II, cyclooxygenase-2, Endoglin, C3 complement, Heat shock protein 27, Xanthine oxidase, Superoxidase dismutase, Endometrial bleeding-assoicated factor and HOX gene. No studies have evaluated the use of these molecular markers as a potential diagnostic/screening tool in endometriosis. The reasons for that are that the level of expression may vary considerably among individuals and biopsy samples, the abnormal expression pattern may be confined to a certain phase in the cycle and that immunostaining is subjective and observer dependant method (Obstet Gynecol Clin North Am. 2003 March; 30(1):95-114, viii-ix).

### SUMMARY OF THE INVENTION

The background art does not teach or suggest markers for endometriosis that are sufficiently sensitive and/or accurate, alone or in combination.

The present invention overcomes these deficiencies of the background art by providing novel markers for endometriosis that are both sensitive and accurate. These markers are overexpressed in endometriosis specifically, as opposed to normal tissues. The measurement of these markers, alone or in combination, in patient (biological) Samples provides information that the diagnostician can correlate with a probable diagnosis of endometriosis. The markers of the present invention, alone or in combination, show a high degree of differential detection between normal and endometriosis states.

According to preferred embodiments of the present invention, examples of suitable biological samples which may optionally be used with preferred embodiments of the present invention include but are not limited to blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, breast tissue, any human organ or tissue, including any tumor or normal tissue, any sample obtained by lavage (for example of the bronchial system or of the uterus), and also samples of in vivo cell culture constituents. In a preferred embodiment, the biological sample comprises uterine tissue, preferably endometrial tissue found anywhere in the pelvic or abdominal cavity and/or a serum sample and/or a urine sample and/or any other tissue or liquid sample. The sample can optionally be diluted with a suitable eluant before contacting the sample to an antibody and/or performing any other diagnostic assay.

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) Signalp_hmm or (iv) Signalp_nn (both from Center for Biological Sequence Analysis, Technical University of Denmark DTU, http://www.cbs.dtu.dk/services/SignalP/background/prediction.php) for signal peptide prediction. The terms “signalphmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amrit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (−). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:
model=sw.model
GAPEXT=0
GAPOP=100.
MATRIX=blosum 100

It should be noted that the terms “segment”, “seg” and “node” are used interchangeably in reference to nucleic acid sequences of the present invention; they refer to portions of nucleic acid sequences that were shown to have one or more properties as described below. They are also the building blocks that were used to construct complete nucleic acid sequences as described in greater detail below. Optionally and preferably, they are examples of oligonucleotides which are embodiments of the present invention, for example as amplicons, hybridization units and/or from which primers and/or complementary oligonucleotides may optionally be derived, and/or for any other use.

As used herein the phrase “endometriosis” refers to any type of endometriosis and/or disease of the endometrium and/or of endometrial tissue.

The term “marker” in the context of the present invention refers to a nucleic acid fragment, a peptide, or a polypeptide, which is differentially present in a sample taken from subjects (patients) Having endometriosis as compared to a comparable sample taken from subjects who do not have endometriosis.

The phrase “differentially present” refers to differences in the quantity of a marker present in a sample taken from patients having endometriosis as compared to a comparable sample taken from patients who do not have endometriosis. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of endometriosis. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient with endometriosis or a person without endometriosis. A control amount can be either in absolute amount (e.g., microgram/ml) or a relative amount (e.g., relative intensity of signals).

“Detect” refers to identifying the presence, absence or amount of the object to be detected.

A “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

According to preferred embodiments of the present invention, there is provided a nucleic acid sequence comprising a sequence from the table below; and/or

Transcript Name

S71513_T2 (SEQ ID NO: 1)

a nucleic acid sequence comprising a sequence from the table below:

Segment Name

S71513_node_0 (SEQ ID NO: 2)
S71513_node_5 (SEQ ID NO: 3)
S71513_node_6 (SEQ ID NO: 4)
S71513_node_8 (SEQ ID NO: 5)
S71513_node_1 (SEQ ID NO: 6)
S71513_node_4 (SEQ ID NO: 7)

According to preferred embodiments of the present invention, there is provided an amino acid sequence comprising a sequence from the table below:

Protein Name

S71513_P2 (SEQ ID NO: 9)

According to preferred embodiments of the present invention, there is provided a nucleic acid sequence comprising a sequence from the table below; and/or

Transcript Name

HUMELAM1A_T1 (SEQ ID NO: 10)
HUMELAM1A_T5 (SEQ ID NO: 11)
HUMELAM1A_T6 (SEQ ID NO: 12)

a nucleic acid sequence comprising a sequence from the table below:

Segment Name

HUMELAM1A_node_5 (SEQ ID NO: 13)
HUMELAM1A_node_8 (SEQ ID NO: 14)
HUMELAM1A_node_10 (SEQ ID NO: 15)
HUMELAM1A_node_11 (SEQ ID NO: 16)
HUMELAM1A_node_13 (SEQ ID NO: 17)
HUMELAM1A_node_15 (SEQ ID NO: 18)
HUMELAM1A_node_18 (SEQ ID NO: 19)
HUMELAM1A_node_19 (SEQ ID NO: 20)
HUMELAM1A_node_20 (SEQ ID NO: 21)
HUMELAM1A_node_22 (SEQ ID NO: 22)
HUMELAM1A_node_33 (SEQ ID NO: 23)
HUMELAM1A_node_0 (SEQ ID NO: 24)
HUMELAM1A_node_2 (SEQ ID NO: 25)
HUMELAM1A_node_7 (SEQ ID NO: 26)
HUMELAM1A_node_24 (SEQ ID NO: 27)
HUMELAM1A_node_26 (SEQ ID NO: 28)
HUMELAM1A_node_29 (SEQ ID NO: 29)

According to preferred embodiments of the present invention, there is provided an amino acid sequence comprising a sequence from the table below:

Protein Name

HUMELAM1A_P2 (SEQ ID NO: 31)
HUMELAM1A_P2 (SEQ ID NO: 32)
HUMELAM1A_P2 (SEQ ID NO: 33)

According to preferred embodiments of the present invention, there is provided a nucleic acid sequence comprising a sequence from the table below; and/or

Transcript Name

HUMHPA1B_PEA_1_T1 (SEQ ID NO: 34)
HUMHPA1B_PEA_1_T4 (SEQ ID NO: 35)
HUMHPA1B_PEA_1_T6 (SEQ ID NO: 36)
HUMHPA1B_PEA_1_T7 (SEQ ID NO: 37)
HUMHPA1B_PEA_1_T12 (SEQ ID NO: 38)
HUMHPA1B_PEA_1_T16 (SEQ ID NO: 39)
HUMHPA1B_PEA_1_T19 (SEQ ID NO: 40)
HUMHPA1B_PEA_1_T20 (SEQ ID NO: 41)
HUMHPA1B_PEA_1_T27 (SEQ ID NO: 42)
HUMHPA1B_PEA_1_T29 (SEQ ID NO: 43)
HUMHPA1B_PEA_1_T55 (SEQ ID NO: 44)
HUMHPA1B_PEA_1_T56 (SEQ ID NO: 45)
HUMHPA1B_PEA_1_T59 (SEQ ID NO: 46)

and/or a nucleic acid sequence comprising a sequence from the table below:

Segment Name

HUMHPA1B_PEA_1_node_20 (SEQ ID
NO: 47)
HUMHPA1B_PEA_1_node_25 (SEQ ID
NO: 48)
HUMHPA1B_PEA_1_node_28 (SEQ ID
NO: 49)
HUMHPA1B_PEA_1_node_35 (SEQ ID
NO: 50)
HUMHPA1B_PEA_1_node_88 (SEQ ID
NO: 51)
HUMHPA1B_PEA_1_node_0 (SEQ ID
NO: 52)
HUMHPA1B_PEA_1_node_1 (SEQ ID
NO: 53)
HUMHPA1B_PEA_1_node_3 (SEQ ID
NO: 54)
HUMHPA1B_PEA_1_node_4 (SEQ ID
NO: 55)
HUMHPA1B_PEA_1_node_5 (SEQ ID
NO: 56)
HUMHPA1B_PEA_1_node_6 (SEQ ID
NO: 57)
HUMHPA1B_PEA_1_node_7 (SEQ ID
NO: 58)
HUMHPA1B_PEA_1_node_10 (SEQ ID
NO: 59)
HUMHPA1B_PEA_1_node_11 (SEQ ID
NO: 60)
HUMHPA1B_PEA_1_node_12 (SEQ ID
NO: 61)
HUMHPA1B_PEA_1_node_13 (SEQ ID
NO: 62)
HUMHPA1B_PEA_1_node_14 (SEQ ID
NO: 63)
HUMHPA1B_PEA_1_node_15 (SEQ ID
NO: 64)
HUMHPA1B_PEA_1_node_16 (SEQ ID
NO: 65)
HUMHPA1B_PEA_1_node_17 (SEQ ID
NO: 66)
HUMHPA1B_PEA_1_node_18 (SEQ ID
NO: 67)
HUMHPA1B_PEA_1_node_19 (SEQ ID
NO: 68)
HUMHPA1B_PEA_1_node_21 (SEQ ID
NO: 69)
HUMHPA1B_PEA_1_node_22 (SEQ ID
NO: 70)
HUMHPA1B_PEA_1_node_23 (SEQ ID
NO: 71)
HUMHPA1B_PEA_1_node_24 (SEQ ID
NO: 72)
HUMHPA1B_PEA_1_node_27 (SEQ ID
NO: 73)
HUMHPA1B_PEA_1_node_29 (SEQ ID
NO: 74)
HUMHPA1B_PEA_1_node_30 (SEQ ID
NO: 75)
HUMHPA1B_PEA_1_node_31 (SEQ ID
NO: 76)
HUMHPA1B_PEA_1_node_32 (SEQ ID
NO: 77)
HUMHPA1B_PEA_1_node_33 (SEQ ID
NO: 78)
HUMHPA1B_PEA_1_node_34 (SEQ ID
NO: 79)
HUMHPA1B_PEA_1_node_36 (SEQ ID
NO: 80)
HUMHPA1B_PEA_1_node_37 (SEQ ID
NO: 81)
HUMHPA1B_PEA_1_node_38 (SEQ ID
NO: 82)
HUMHPA1B_PEA_1_node_39 (SEQ ID
NO: 83)
HUMHPA1B_PEA_1_node_40 (SEQ ID
NO: 84)
HUMHPA1B_PEA_1_node_41 (SEQ ID
NO: 85)
HUMHPA1B_PEA_1_node_42 (SEQ ID
NO: 86)
HUMHPA1B_PEA_1_node_43 (SEQ ID
NO: 87)
HUMHPA1B_PEA_1_node_44 (SEQ ID
NO: 88)
HUMHPA1B_PEA_1_node_45 (SEQ ID
NO: 89)
HUMHPA1B_PEA_1_node_46 (SEQ ID
NO: 90)
HUMHPA1B_PEA_1_node_47 (SEQ ID
NO: 91)
HUMHPA1B_PEA_1_node_48 (SEQ ID
NO: 92)
HUMHPA1B_PEA_1_node_49 (SEQ ID
NO: 93)
HUMHPA1B_PEA_1_node_50 (SEQ ID
NO: 94)
HUMHPA1B_PEA_1_node_51 (SEQ ID
NO: 95)
HUMHPA1B_PEA_1_node_52 (SEQ ID
NO: 96)
HUMHPA1B_PEA_1_node_53 (SEQ ID
NO: 97)
HUMHPA1B_PEA_1_node_54 (SEQ ID
NO: 98)
HUMHPA1B_PEA_1_node_55 (SEQ ID
NO: 99)
HUMHPA1B_PEA_1_node_56 (SEQ ID
NO: 100)
HUMHPA1B_PEA_1_node_57 (SEQ ID
NO: 101)
HUMHPA1B_PEA_1_node_58 (SEQ ID
NO: 102)
HUMHPA1B_PEA_1_node_59 (SEQ ID
NO: 103)
HUMHPA1B_PEA_1_node_60 (SEQ ID
NO: 104)
HUMHPA1B_PEA_1_node_61 (SEQ ID
NO: 105)
HUMHPA1B_PEA_1_node_62 (SEQ ID
NO: 106)
HUMHPA1B_PEA_1_node_63 (SEQ ID
NO: 107)
HUMHPA1B_PEA_1_node_64 (SEQ ID
NO: 108)
HUMHPA1B_PEA_1_node_65 (SEQ ID
NO: 109)
HUMHPA1B_PEA_1_node_66 (SEQ ID
NO: 110)
HUMHPA1B_PEA_1_node_67 (SEQ ID
NO: 111)
HUMHPA1B_PEA_1_node_69 (SEQ ID
NO: 112)
HUMHPA1B_PEA_1_node_70 (SEQ ID
NO: 113)
HUMHPA1B_PEA_1_node_71 (SEQ ID
NO: 114)
HUMHPA1B_PEA_1_node_72 (SEQ ID
NO: 115)
HUMHPA1B_PEA_1_node_73 (SEQ ID
NO: 116)
HUMHPA1B_PEA_1_node_74 (SEQ ID
NO: 117)
HUMHPA1B_PEA_1_node_75 (SEQ ID
NO: 118)
HUMHPA1B_PEA_1_node_76 (SEQ ID
NO: 119)
HUMHPA1B_PEA_1_node_77 (SEQ ID
NO: 120)
HUMHPA1B_PEA_1_node_78 (SEQ ID
NO: 121)
HUMHPA1B_PEA_1_node_79 (SEQ ID
NO: 122)
UMHPA1B_PEA_1_node_80 (SEQ ID
NO: 123)
HUMHPA1B_PEA_1_node_81 (SEQ ID
NO: 124)
HUMHPA1B_PEA_1_node_82 (SEQ ID
NO: 125)
HUMHPA1B_PEA_1_node_83 (SEQ ID
NO: 126)
HUMHPA1B_PEA_1_node_84 (SEQ ID
NO: 127)
HUMHPA1B_PEA_1_node_85 (SEQ ID
NO: 128)
HUMHPA1B_PEA_1_node_86 (SEQ ID
NO: 129)
HUMHPA1B_PEA_1_node_87 (SEQ ID
NO: 130)

According to preferred embodiments of the present invention, there is provided an amino acid sequence comprising a sequence from the table below:

Protein Name

HUMHPA1B_PEA_1_P61 (SEQ ID NO: 133)
HUMHPA1B_PEA_1_P62 (SEQ ID NO: 134)
HUMHPA1B_PEA_1_P64 (SEQ ID NO: 135)
HUMHPA1B_PEA_1_P65 (SEQ ID NO: 136)
HUMHPA1B_PEA_1_P68 (SEQ ID NO: 137)
HUMHPA1B_PEA_1_P72 (SEQ ID NO: 138)
HUMHPA1B_PEA_1_P75 (SEQ ID NO: 139)
HUMHPA1B_PEA_1_P76 (SEQ ID NO: 140)
HUMHPA1B_PEA_1_P81 (SEQ ID NO: 141)
HUMHPA1B_PEA_1_P83 (SEQ ID NO: 142)
HUMHPA1B_PEA_1_P106 (SEQ ID
NO: 143)
HUMHPA1B_PEA_1_P107 (SEQ ID
NO: 144)
HUMHPA1B_PEA_1_P115 (SEQ ID
NO: 145)

According to preferred embodiments of the present invention, there is provided a nucleic acid sequence comprising a sequence from the table below; and/or

Transcript Name

HSHGFR_T1 (SEQ ID NO: 146)
HSHGFR_T6 (SEQ ID NO: 147)
HSHGFR_T8 (SEQ ID NO: 148)
HSHGFR_T13 (SEQ ID NO: 149)
HSHGFR_T14 (SEQ ID NO: 150)

a nucleic acid sequence comprising a sequence from the table below:

Segment Name

HSHGFR_node_2 (SEQ ID NO: 151)
HSHGFR_node_2 (SEQ ID NO: 152)
HSHGFR_node_6 (SEQ ID NO: 153)
HSHGFR_node_11 (SEQ ID NO: 154)
HSHGFR_node_15 (SEQ ID NO: 155)
HSHGFR_node_16 (SEQ ID NO: 156)
HSHGFR_node_18 (SEQ ID NO: 157)
HSHGFR_node_22 (SEQ ID NO: 158)
HSHGFR_node_24 (SEQ ID NO: 159)
HSHGFR_node_8 (SEQ ID NO: 160)
HSHGFR_node_10 (SEQ ID NO: 161)
HSHGFR_node_14 (SEQ ID NO: 162)
HSHGFR_node_20 (SEQ ID NO: 163)

According to preferred embodiments of the present invention, there is provided an amino acid sequence comprising a sequence from the table below:

Protein Name

HSHGFR_P6 (SEQ ID NO: 165)
HSHGFR_P11 (SEQ ID NO: 166)
HSHGFR_P12 (SEQ ID NO: 167)
HSHGFR_P13 (SEQ ID NO: 168)

According to preferred embodiments of the present invention, there is provided a nucleic acid sequence comprising a sequence from the table below; and/or

Transcript Name

S56892_PEA_1_T3 (SEQ ID NO: 169)
S56892_PEA_1_T9 (SEQ ID NO: 170)
S56892_PEA_1_T10 (SEQ ID NO: 171)
S56892_PEA_1_T13 (SEQ ID NO: 172)

a nucleic acid sequence comprising a sequence from the table below:

Segment Name

S56892_PEA_1_node_0 (SEQ ID NO: 173)
S56892_PEA_1_node_5 (SEQ ID NO: 174)
S56892_PEA_1_node_10 (SEQ ID NO: 175)
S56892_PEA_1_node_18 (SEQ ID NO: 176)
S56892_PEA_1_node_21 (SEQ ID NO: 177)
S56892_PEA_1_node_3 (SEQ ID NO: 178)
S56892_PEA_1_node_4 (SEQ ID NO: 179)
S56892_PEA_1_node_6 (SEQ ID NO: 180)
S56892_PEA_1_node_7 (SEQ ID NO: 181)
S56892_PEA_1_node_8 (SEQ ID NO: 182)
S56892_PEA_1_node_9 (SEQ ID NO: 183)
S56892_PEA_1_node_12 (SEQ ID NO: 184)
S56892_PEA_1_node_13 (SEQ ID NO: 185)
S56892_PEA_1_node_14 (SEQ ID NO: 186)
S56892_PEA_1_node_16 (SEQ ID NO: 187)
S56892_PEA_1_node_17 (SEQ ID NO: 188)
S56892_PEA_1_node_19 (SEQ ID NO: 189)
S56892_PEA_1_node_20 (SEQ ID NO: 190)
S56892_PEA_1_node_22 (SEQ ID NO: 191)
S56892_PEA_1_node_23 (SEQ ID NO: 192)

According to preferred embodiments, there is provided an amino acid sequence comprising a sequence from the table below:

Protein Name

S56892_PEA_1_P2 (SEQ ID NO: 194)
S56892_PEA_1_P8 (SEQ ID NO: 195)
S56892_PEA_1_P9 (SEQ ID NO: 196)
S56892_PEA_1_P11 (SEQ ID NO: 197)

According to preferred embodiments of the present invention, there is provided a nucleic acid sequence comprising a sequence from the table below; and/or

Transcript Name

HSIGFACI_PEA_1_T9 (SEQ ID NO: 198)
HSIGFACI_PEA_1_T10 (SEQ ID NO: 199)
HSIGFACI_PEA_1_T12 (SEQ ID NO: 200)
HSIGFACI_PEA_1_T15 (SEQ ID NO: 201)
HSIGFACI_PEA_1_T16 (SEQ ID NO: 202)
HSIGFACI_PEA_1_T17 (SEQ ID NO: 203)

a nucleic acid sequence comprising a sequence from the table below:

Segment Name

HSIGFACI_PEA_1_node_0 (SEQ ID NO: 204)
HSIGFACI_PEA_1_node_2 (SEQ ID NO: 205)
HSIGFACI_PEA_1_node_6 (SEQ ID NO: 206)
HSIGFACI_PEA_1_node_9 (SEQ ID NO: 207)
HSIGFACI_PEA_1_node_11 (SEQ ID
NO: 208)
HSIGFACI_PEA_1_node_14 (SEQ ID
NO: 209)
HSIGFACI_PEA_1_node_19 (SEQ ID
NO: 210)
HSIGFACI_PEA_1_node_20 (SEQ ID
NO: 211)
HSIGFACI_PEA_1_node_21 (SEQ ID
NO: 212)
HSIGFACI_PEA_1_node_24 (SEQ ID
NO: 213)
HSIGFACI_PEA_1_node_25 (SEQ ID
NO: 214)
HSIGFACI_PEA_1_node_26 (SEQ ID
NO: 215)
HSIGFACI_PEA_1_node_27 (SEQ ID
NO: 216)
HSIGFACI_PEA_1_node_13 (SEQ ID
NO: 217)
HSIGFACI_PEA_1_node_22 (SEQ ID
NO: 218)
HSIGFACI_PEA_1_node_23 (SEQ ID
NO: 219)

According to preferred embodiments of the present invention, there is provided an amino acid sequence comprising a sequence from the table below:

Protein Name

HSIGFACI_PEA_1_P5 (SEQ ID NO: 225)
HSIGFACI_PEA_1_P2 (SEQ ID NO: 226)
HSIGFACI_PEA_1_P6 (SEQ ID NO: 227)
HSIGFACI_PEA_1_P1 (SEQ ID NO: 228)
HSIGFACI_PEA_1_P7 (SEQ ID NO: 229)
HSIGFACI_PEA_1_P8 (SEQ ID NO: 230)

According to preferred embodiments of the present invention, there is provided a nucleic acid sequence comprising a sequence from the table below; and/or

Transcript Name
HSSTROMR_PEA_1_T3 (SEQ ID NO: 231)

a nucleic acid sequence comprising a sequence from the table below:

Segment Name

HSSTROMR_PEA_1_node_0 (SEQ ID
NO: 232)
HSSTROMR_PEA_1_node_5 (SEQ ID
NO: 233)
HSSTROMR_PEA_1_node_7 (SEQ ID
NO: 234)
HSSTROMR_PEA_1_node_9 (SEQ ID
NO: 235)
HSSTROMR_PEA_1_node_13 (SEQ ID
NO: 236)
HSSTROMR_PEA_1_node_16 (SEQ ID
NO: 237)
HSSTROMR_PEA_1_node_18 (SEQ ID
NO: 238)
HSSTROMR_PEA_1_node_20 (SEQ ID
NO: 239)
HSSTROMR_PEA_1_node_28 (SEQ ID
NO: 240)
HSSTROMR_PEA_1_node_14 (SEQ ID
NO: 241)
HSSTROMR_PEA_1_node_22 (SEQ ID
NO: 242)

According to preferred embodiments of the present invention, there is provided an amino acid sequence comprising a sequence from the table below:

Protein Name

HSSTROMR_PEA_1_P4 (SEQ ID NO: 244)

According to preferred embodiments of the present invention, there is provided a nucleic acid sequence comprising a sequence from the table below; and/or

Transcript Name

HUM4COLA_PEA_1_T1 (SEQ ID NO: 245)
HUM4COLA_PEA_1_T5 (SEQ ID NO: 246)
HUM4COLA_PEA_1_T6 (SEQ ID NO: 247)

a nucleic acid sequence comprising a sequence from the table below:

Segment Name

HUM4COLA_PEA_1_node_0 (SEQ ID
NO: 248)
HUM4COLA_PEA_1_node_0 (SEQ ID
NO: 249)
HUM4COLA_PEA_1_node_4 (SEQ ID
NO: 250)
HUM4COLA_PEA_1_node_7 (SEQ ID
NO: 251)
HUM4COLA_PEA_1_node_11 (SEQ ID
NO: 252)
HUM4COLA_PEA_1_node_19 (SEQ ID
NO: 253)
HUM4COLA_PEA_1_node_40 (SEQ ID
NO: 254)
HUM4COLA_PEA_1_node_41 (SEQ ID
NO: 255)
HUM4COLA_PEA_1_node_8 (SEQ ID
NO: 256)
HUM4COLA_PEA_1_node_9 (SEQ ID
NO: 257)
HUM4COLA_PEA_1_node_10 (SEQ ID
NO: 258)
HUM4COLA_PEA_1_node_12 (SEQ ID
NO: 259)
HUM4COLA_PEA_1_node_13 (SEQ ID
NO: 260)
HUM4COLA_PEA_1_node_16 (SEQ ID
NO: 261)
HUM4COLA_PEA_1_node_17 (SEQ ID
NO: 262)
HUM4COLA_PEA_1_node_22 (SEQ ID
NO: 263)
HUM4COLA_PEA_1_node_23 (SEQ ID
NO: 264)
HUM4COLA_PEA_1_node_24 (SEQ ID
NO: 265)
HUM4COLA_PEA_1_node_25 (SEQ ID
NO: 266)
HUM4COLA_PEA_1_node_26 (SEQ ID
NO: 267)
HUM4COLA_PEA_1_node_27 (SEQ ID
NO: 268)
HUM4COLA_PEA_1_node_29 (SEQ ID
NO: 269)
HUM4COLA_PEA_1_node_30 (SEQ ID
NO: 270)
HUM4COLA_PEA_1_node_32 (SEQ ID
NO: 271)
HUM4COLA_PEA_1_node_33 (SEQ ID
NO: 272)
HUM4COLA_PEA_1_node_36 (SEQ ID
NO: 273)
HUM4COLA_PEA_1_node_37 (SEQ ID
NO: 274)

According to preferred embodiments of the present invention, there is provided an amino acid sequence comprising a sequence from the table below:

Protein Name

HUM4COLA_PEA_1_P7 (SEQ ID NO: 276)
HUM4COLA_PEA_1_P14 (SEQ ID NO: 277)
HUM4COLA_PEA_1_P15 (SEQ ID NO: 278)

According to preferred embodiments of the present invention, there is provided a nucleic acid sequence comprising a sequence from the table below; and/or

Transcript Name

HUMICAMA1A_PEA_1_T2 (SEQ ID
NO: 279)
HUMICAMA1A_PEA_1_T4 (SEQ ID
NO: 280)
HUMICAMA1A_PEA_1_T5 (SEQ ID
NO: 281)
HUMICAMA1A_PEA_1_T8 (SEQ ID
NO: 282)
HUMICAMA1A_PEA_1_T12 (SEQ ID
NO: 283)
HUMICAMA1A_PEA_1_T16 (SEQ ID
NO: 284)

a nucleic acid sequence comprising a sequence from the table below:

Segment Name

HUMICAMA1A_PEA_1_node_0 (SEQ ID
NO: 285)
HUMICAMA1A_PEA_1_node_3 (SEQ ID
NO: 286)
HUMICAMA1A_PEA_1_node_12 (SEQ ID
NO: 287)
HUMICAMA1A_PEA_1_node_13 (SEQ ID
NO: 288)
HUMICAMA1A_PEA_1_node_14 (SEQ ID
NO: 289)
HUMICAMA1A_PEA_1_node_20 (SEQ ID
NO: 290)
HUMICAMA1A_PEA_1_node_21 (SEQ ID
NO: 291)
HUMICAMA1A_PEA_1_node_24 (SEQ ID
NO: 292)
HUMICAMA1A_PEA_1_node_25 (SEQ ID
NO: 293)
HUMICAMA1A_PEA_1_node_27 (SEQ ID
NO: 294)
HUMICAMA1A_PEA_1_node_29 (SEQ ID
NO: 295)
HUMICAMA1A_PEA_1_node_2 (SEQ ID
NO: 296)
HUMICAMA1A_PEA_1_node_4 (SEQ ID
NO: 297)
HUMICAMA1A_PEA_1_node_15 (SEQ ID
NO: 298)
HUMICAMA1A_PEA_1_node_16 (SEQ ID
NO: 299)
HUMICAMA1A_PEA_1_node_17 (SEQ ID
NO: 300)
HUMICAMA1A_PEA_1_node_18 (SEQ ID
NO: 301)
HUMICAMA1A_PEA_1_node_19 (SEQ ID
NO: 302)
HUMICAMA1A_PEA_1_node_22 (SEQ ID
NO: 303)
HUMICAMA1A_PEA_1_node_23 (SEQ ID
NO: 304)
HUMICAMA1A_PEA_1_node_26 (SEQ ID
NO: 305)
HUMICAMA1A_PEA_1_node_28 (SEQ ID
NO: 306)

According to preferred embodiments of the present invention, there is provided an amino acid sequence comprising a sequence from the table below:

Protein Name

HUMICAMA1A_PEA_1_P2 (SEQ ID
NO: 309)
HUMICAMA1A_PEA_1_P5 (SEQ ID
NO: 310)
HUMICAMA1A_PEA_1_P8 (SEQ ID
NO: 311)
HUMICAMA1A_PEA_1_P15 (SEQ ID
NO: 312)

According to preferred embodiments of the present invention, there is provided a nucleic acid sequence comprising a sequence from the table below; and/or

Transcript Name

HUMLYSYL_PEA_1_T2 (SEQ ID NO: 313)
HUMLYSYL_PEA_1_T4 (SEQ ID NO: 314)
HUMLYSYL_PEA_1_T5 (SEQ ID NO: 315)
HUMLYSYL_PEA_1_T6 (SEQ ID NO: 316)
HUMLYSYL_PEA_1_T8 (SEQ ID NO: 317)
HUMLYSYL_PEA_1_T9 (SEQ ID NO: 318)
HUMLYSYL_PEA_1_T19 (SEQ ID NO: 319)
HUMLYSYL_PEA_1_T20 (SEQ ID NO: 320)
HUMLYSYL_PEA_1_T22 (SEQ ID NO: 321)
HUMLYSYL_PEA_1_T24 (SEQ ID NO: 322)

a nucleic acid sequence comprising a sequence from the table below:

Segment Name

HUMLYSYL_PEA_1_node_6 (SEQ ID
NO: 323)
HUMLYSYL_PEA_1_node_14 (SEQ ID
NO: 324)
HUMLYSYL_PEA_1_node_19 (SEQ ID
NO: 325)
HUMLYSYL_PEA_1_node_38 (SEQ ID
NO: 326)
HUMLYSYL_PEA_1_node_55 (SEQ ID
NO: 327)
HUMLYSYL_PEA_1_node_59 (SEQ ID
NO: 328)
HUMLYSYL_PEA_1_node_61 (SEQ ID
NO: 329)
HUMLYSYL_PEA_1_node_62 (SEQ ID
NO: 330)
HUMLYSYL_PEA_1_node_65 (SEQ ID
NO: 331)
HUMLYSYL_PEA_1_node_71 (SEQ ID
NO: 332)
HUMLYSYL_PEA_1_node_72 (SEQ ID
NO: 333)
HUMLYSYL_PEA_1_node_3 (SEQ ID
NO: 334)
HUMLYSYL_PEA_1_node_4 (SEQ ID
NO: 335)
HUMLYSYL_PEA_1_node_8 (SEQ ID
NO: 336)
HUMLYSYL_PEA_1_node_10 (SEQ ID
NO: 337)
HUMLYSYL_PEA_1_node_11 (SEQ ID
NO: 338)
HUMLYSYL_PEA_1_node_12 (SEQ ID
NO: 339)
HUMLYSYL_PEA_1_node_16 (SEQ ID
NO: 340)
HUMLYSYL_PEA_1_node_20 (SEQ ID
NO: 341)
HUMLYSYL_PEA_1_node_23 (SEQ ID
NO: 342)
HUMLYSYL_PEA_1_node_25 (SEQ ID
NO: 343)
HUMLYSYL_PEA_1_node_28 (SEQ ID
NO: 344)
HUMLYSYL_PEA_1_node_30 (SEQ ID
NO: 345)
HUMLYSYL_PEA_1_node_31 (SEQ ID
NO: 346)
HUMLYSYL_PEA_1_node_33 (SEQ ID
NO: 347)
HUMLYSYL_PEA_1_node_34 (SEQ ID
NO: 348)
HUMLYSYL_PEA_1_node_36 (SEQ ID
NO: 349)
HUMLYSYL_PEA_1_node_40 (SEQ ID
NO: 350)
HUMLYSYL_PEA_1_node_41 (SEQ ID
NO: 351)
HUMLYSYL_PEA_1_node_42 (SEQ ID
NO: 352)
HUMLYSYL_PEA_1_node_44 (SEQ ID
NO: 353)
HUMLYSYL_PEA_1_node_45 (SEQ ID
NO: 354)
HUMLYSYL_PEA_1_node_46 (SEQ ID
NO: 355)
HUMLYSYL_PEA_1_node_48 (SEQ ID
NO: 356)
HUMLYSYL_PEA_1_node_49 (SEQ ID
NO: 357)
HUMLYSYL_PEA_1_node_52 (SEQ ID
NO: 358)
HUMLYSYL_PEA_1_node_53 (SEQ ID
NO: 359)
HUMLYSYL_PEA_1_node_56 (SEQ ID
NO: 360)
HUMLYSYL_PEA_1_node_63 (SEQ ID
NO: 361)
HUMLYSYL_PEA_1_node_64 (SEQ ID
NO: 362)
HUMLYSYL_PEA_1_node_66 (SEQ ID
NO: 363)
HUMLYSYL_PEA_1_node_67 (SEQ ID
NO: 364)
HUMLYSYL_PEA_1_node_68 (SEQ ID
NO: 365)
HUMLYSYL_PEA_1_node_70 (SEQ ID
NO: 366)

According to preferred embodiments of the present invention, there is provided an amino acid sequence comprising a sequence from the table below:

Pretein Name

HUMLYSYL_PEA_1_P2 (SEQ ID NO: 369)
HUMLYSYL_PEA_1_P4 (SEQ ID NO: 370)
HUMLYSYL_PEA_1_P5 (SEQ ID NO: 371)
HUMLYSYL_PEA_1_P6 (SEQ ID NO: 372)
HUMLYSYL_PEA_1_P7 (SEQ ID NO: 373)
HUMLYSYL_PEA_1_P13 (SEQ ID NO: 374)
HUMLYSYL_PEA_1_P14 (SEQ ID NO: 375)
HUMLYSYL_PEA_1_P16 (SEQ ID NO: 376)
HUMLYSYL_PEA_1_P18 (SEQ ID NO: 377)
HUMLYSYL_PEA_1_P24 (SEQ ID NO: 378)

According to preferred embodiments of the present invention, preferably any of the above nucleic acid and/or amino acid sequences further comprises any sequence having at least about 70%, preferably at least about 80%, more preferably at least about 90%, most preferably at least about 95% homology thereto.

According to preferred embodiments of the present invention, there is provided an isolated chimeric polypeptide encoding for HUMLYSYL_PEA_{—}1_P2 (SEQ ID NO:369), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQ corresponding to amino acids 1-490 of PLO1_HUMAN_V1 (SEQ ID NO:368), which also corresponds to amino acids 1-490 of HUMLYSYL_PEA_{—}1_P2 (SEQ ID NO:369), and a second amino acid sequence being at least 70%, optionally at least 80%, preferably at least 85%, more preferably at least 90% and most preferably at least 95% homologous to a polypeptide having the sequence VSQERAAQDALWMGQAGRMCSCS (SEQ ID NO:474) corresponding to amino acids 491-513 of HUMLYSYL_PEA_{—}1_P2 (SEQ ID NO:369), wherein said first amino acid sequence and second amino acid sequence are contiguous and in a sequential order.

According to preferred embodiments of the present invention, there is provided an isolated polypeptide encoding for a tail of HUMLYSYL_PEA_{—}1_P2 (SEQ ID NO:369), comprising a polypeptide being at least 70%, optionally at least about 80%, preferably at least about 85%, more preferably at least about 90% and most preferably at least about 95% homologous to the sequence VSQERAAQDALWMGQAGRMCSCS (SEQ ID NO:474) in HUMLYSYL_PEA_{—}1_P2 (SEQ ID NO:369).

According to preferred embodiments of the present invention, there is provided an isolated chimeric polypeptide encoding for HUMLYSYL_PEA_{—}1_P4 (SEQ ID NO:370), comprising a first amino acid sequence being at least 90% homologous to MRPLLLLALLGWLLLAEAKGDAKPE corresponding to amino acids 1-25 of PLO1_HUMAN_V1 (SEQ ID NO:3681, which also corresponds to amino acids 1-25 of HUMLYSYL_PEA_{—}1_P4 (SEQ ID NO:370), a second amino acid sequence being at least 70%, optionally at least 80%, preferably at least 85%, more preferably at least 90% and most preferably at least 95% homologous to a polypeptide having the sequence APCCQEGLRAGGSGSLHLGRDFTVLAGARGSPSPSVSSIPRFWIPGS (SEQ ID NO:504) corresponding to amino acids 26-72 of HUMLYSYL_PEA_{—}1_P4 (SEQ ID NO:370), and a third amino acid sequence being at least 90% homologous to DNLLVLTVATKETEGFRRFKRSAQFFNYKIQALGLGEDWNVEKGTSAGGGQKVRLLK KALEKHADKEDLVILFADSYDVLFASGPRELLKKFRQARSQVVFSAEELIYPDRRLETK YPVVSDGKRFLGSGGFIGYAPNLSKLVAEWEGQDSDSDQLFYTKIFLDPEKREQINITLD HRCRIFQNLDGALDEVVLKFEMGHVRARNLAYDTLPVLIHGNGPTKLQLNYLGNYIPR FWTFETGCTVCDEGLRSLKGIGDEALPTVLVGVFIEQPTPFVSLFFQRLLRLHYPQKHMR LFIHNHEQHHKAQVEEFLAQHGSEYQSVKLVGPEVRMANADARNMGADLCRQDRSCT YYFSVDADVALTEPNSLRLLIQQNKNVIAPLMTRHGRLWSNFWGALSADGYYARSED YVDIVQGRRVGVWNVPYISNIYLIKGSALRGELQSSDLFHHSKLDPDMAFCANIRQQDV FMFLTNRHTLGHLLSLDSYRTTHLHNDLWEVFSNPEDWKEKYIHQNYTKALAGKLVET PCPDVYWFPIFTEVACDELVEEMEHFGQWSLGNNKDNRIQGGYENVPTIDIHMNQIGFE REWHKFLLEYIAPMTEKLYPGYYTRAQFDLAFVVRYKPDEQPSLMPHHDASTFTINIAL NRVGVDYEGGGCRFLRYNCSIRAPRKGWTLMHPGRLTHYHEGLPTTRGTRYIAVSFVD P corresponding to amino acids 26-727 of PLO1_HUMAN_V1 (SEQ ID NO:368), which also corresponds to amino acids 73-774 of HUMLYSYL_PEA_{—}1_P4 (SEQ ID NO:370), wherein said first amino acid sequence, second amino acid sequence and third amino acid sequence are contiguous and in a sequential order.

According to preferred embodiments of the present invention, there is provided an isolated polypeptide encoding for an edge portion of HUMLYSYL_PEA_{—}1_P4 (SEQ ID NO:370), comprising an amino acid sequence being at least 70%, optionally at least about 80%, preferably at least about 85%, more preferably at least about 90% and most preferably at least about 95% homologous to the sequence encoding for APCCQEGLRAGGSGSLHLGRDFTVLAGARGSPSPSVSSIPRFWIPGS (SEQ ID NO:504), corresponding to HUMLYSYL_PEA_{—}1_P4 (SEQ ID NO:370).

According to preferred embodiments of the present invention, there is provided an isolated chimeric polypeptide encoding for HUMLYSYL_PEA_{—}1_P5 (SEQ ID NO:371), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIG corresponding to amino acids 1-281 of PLO1_HUMAN_V1 (SEQ ID NO:368), which also corresponds to amino acids 1-281 of HUMLYSYL_PEA_{—}1_P5 (SEQ ID NO:371), and a second amino acid sequence being at least 90% homologous to RLLRLHYPQKHMRLFIHNHEQHHKAQVEEFLAQHGSEYQSVKLVGPEVRMANADARN MGADLCRQDRSCTYYFSVDADVALTEPNSLRLLIQQNKNVIAPLMTRHGRLWSNFWG ALSADGYYARSEDYVDIVQGRRVGVWNVPYISNIYLIKGSALRGELQS SDLFHHSKLDP DMAFCANIRQQDVFMFLTNRHTLGHLLSLDSYRTTHLHNDLWEVFSNPEDWKEKYIH QNYTKALAGKLVETPCPDVYWFPIFTEVACDELVEEMEHFGQWSLGNNKDNRIQGGY ENVPTIDIHMNQIGFEREWHKFLLEYIAPMTEKLYPGYYTRAQFDLAFVVRYKPDEQPS LMPHHDASTFTINIALNRVGVDYEGGGCRFLRYNCSIRAPRKGWTLMHPGRLTHYHEG LPTTRGTRYIAVSFVDP corresponding to amino acids 307-727 of PLO1_HUMAN_V1 (SEQ ID NO:368), which also corresponds to amino acids 282-702 of HUMLYSYL_PEA_{—}1_P5 (SEQ ID NO:371), wherein said first amino acid sequence and second amino acid sequence are contiguous and in a sequential order.

According to preferred embodiments of the present invention, there is provided an isolated chimeric polypeptide encoding for an edge portion of HUMLYSYL_PEA_{—}1_P5 (SE ID NO:371), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GR, having a structure as follows: a sequence starting from any of amino acid numbers 281−x to 281; and ending at any of amino acid numbers 282+((n−2)−x), in which x varies from 0 to n−2.

According to preferred embodiments of the present invention, there is provided an isolated chimeric polypeptide encoding for HUMLYSYL_PEA_{—}1_P6 (SEQ ID NO:372), comprising a first amino acid sequence being at least 90% homologous to MRPLLLLALLGWLLLAEAKGDAKPEDNLLVLTVATKETEGFRRFKRSAQFFNYKI corresponding to amino acids 1-55 of PLO1_HUMAN_V1 (SEQ ID NO:368), which also corresponds to amino acids 1-55 of HUMLYSYL_PEA_{—}1_P6 (SEQ ID NO:372), a second amino acid sequence being at least 70%, optionally at least 80%, preferably at least 85%, more preferably at least 90% and most preferably at least 95% homologous to a polypeptide having the sequence QPVLRGVSL (SEQ ID NO:505) corresponding to amino acids 56-64 of HUMLYSYL_PEA_{—}1_P6 (SEQ ID NO:372), and a third amino acid sequence being at least 90% homologous to QALGLGEDWNVEKGTSAGGGQKVRLLKKALEKHADKEDLVILFADSYDVLFASGPRE LLKKFRQARSQVVFSAEELIYPDRRLETKYPVVSDGKRFLGSGGFIGYAPNLSKLVAEW EGQDSDSDQLFYTKIFLDPEKREQINITLDHRCRIFQNLDGALDEVVLKFEMGHVRARN LAYDTLPVLIHGNGPTKLQLNYLGNYIPRFWTFETGCTVCDEGLRSLKGIGDEALPTVL VGVFIEQPTPFVSLFFQRLLRLHYPQKHMRLFIHNHEQHHKAQVEEFLAQHGSEYQSVK LVGPEVRMANADARNMGADLCRQDRSCTYYFSVDADVALTEPNSLRLLIQQNKNVIA PLMTRHGRLWSNFWGALSADGYYARSEDYVDIVQGRRVGVWNVPYISNIYLIKGSALR GELQS SDLFHHSKLDPDMAFCANIRQQDVFMFLTNRHTLGHLLSLDSYRTTHLHNDLW EVFSNPEDWKEKYIHQNYTKALAGKLVETPCPDVYWFPIFTEVACDELVEEMEHFGQW SLGNNKDNRIQGGYENVPTIDIHMNQIGFEREWHKFLLEYIAPMTEKLYPGYYTRAQFD LAFVVRYKPDEQPSLMPHHDASTFTINIALNRVGVDYEGGGCRFLRYNCSIAAPRKGW TLMHPGRLTHYHEGLPTTRGTRYIAVSFVDP corresponding to amino acids 56-727 of PLO1_HUMAN_V1 (SEQ ID NO:368), which also corresponds to amino acids 65-736 of HUMLYSYL_PEA_{—}1_P6 (SEQ ID NO:372), wherein said first amino acid sequence, second amino acid sequence and third amino acid sequence are contiguous and in a sequential order.

According to preferred embodiments of the present invention, there is provided an isolated polypeptide encoding for an edge portion of HUMLYSYL_PEA_{—}1_P6 (SEQ ID NO:372), comprising an amino acid sequence being at least 70%, optionally at least about 80%, preferably at least about 85%, more preferably at least about 90% and most preferably at least about 95% homologous to the sequence encoding for QPVLRGVSL (SEQ ID NO:505), corresponding to HUMLYSYL_PEA_{—}1_P6 (SEQ ID NO:372).

According to preferred embodiments of the present invention, there is provided an isolated chimeric polypeptide encoding for HUMLYSYL_PEA_{—}1_P7 (SEQ ID NO:373), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGAL corresponding to amino acids 1-214 of PLO1_HUMAN_V1 (SEQ ID NO:368), which also corresponds to amino acids 1-214 of HUMLYSYL_PEA_{—}1_P7 (SEQ ID NO:373), a second amino acid sequence being at least 70%, optionally at least 80%, preferably at least 85%, more preferably at least 90% and most preferably at least 95% homologous to a polypeptide having the sequence VSPWGQGHLPGACYELTASVLTSELSVMPSFPA (SEQ ID NO:506) corresponding to amino acids 215-247 of HUMLYSYL_PEA_{—}1_P7 (SEQ ID NO:373), a third amino acid sequence being at least 90% homologous to VV corresponding to amino acids 217-218 of PLO1_HUMAN_V1 (SEQ ID NO:368), which also corresponds to amino acids 248-249 of HUMLYSYL_PEA_{—}1_P7 (SEQ ID NO:373), and a fourth amino acid sequence being at least 90% homologous to LQLNYLGNYIPRFWTFETGCTVCDEGLRSLKGIGDEALPTVLVGVFIEQPTPFVSLFFQR LLRLHYPQKHMRLFIHNHEQHHKAQVEEFLAQHGSEYQSVKLVGPEVRMANADARN MGADLCRQDRSCTYYFSVDADVALTEPNSLRLLIQQNKNVIAPLMTRHGRLWSNFWG ALSADGYYARSEDYVDIVQGRRVGVWNVPYISNIYLIKGSALRGELQS SDLFHHSKLDP DMAFCANIRQQDVFMFLTNRHTLGHLLSLDSYRTTHLHNDLWEVFSNPEDWKEKYIH QNYTKALAGKLVETPCPDVYWFPIFTEVACDELVEEMEHFGQWSLGNNKDNRIQGGY ENVPTIDIHMNQIGFEREWHKFLLEYIAPMTEKLYPGYYTRAQFDLAFVVRYKPDEQPS LMPHHDASTFTINIALNRVGVDYEGGGCRFLRYNCSIRAPRKGWTLMHPGRLTHYHEG LPTTRGTRYIAVSFVDP corresponding to amino acids 248-727 of PLO1_HUMAN_V1 (SEQ ID NO:368), which also corresponds to amino acids 250-729 of HUMLYSYL_PEA_{—}1_P7 (SEQ ID NO:373), wherein said first amino acid sequence, second amino acid sequence, third amino acid sequence and fourth amino acid sequence are contiguous and in a sequential order.

According to preferred embodiments of the present invention, there is provided an isolated polypeptide encoding for an edge portion of HUMLYSYL_PEA_{—}1_P7 (SEQ ID NO:373), comprising an amino acid sequence being at least 70%, optionally at least about 80%, preferably at least about 85%, more preferably at least about 90% and most preferably at least about 95% homologous to the sequence encoding for VSPWGQGHLPGACYELTASVLTSELSVMPSFPA (SEQ ID NO:506), corresponding to HUMLYSYL_PEA_{—}1_P7 (SEQ ID NO:373).

According to preferred embodiments of the present invention, there is provided a bridge portion of HUMLYSYL_PEA_{—}1_P7 (SEQ ID NO:373), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise LV, having a structure as follows (numbering according to HUMLYSYL_PEA_{—}1_P7 (SEQ ID NO:373)): a sequence starting from any of amino acid numbers 214−x to 214; and ending at any of amino acid numbers 215+((n−2)−x), in which x varies from 0 to n−2.

According to preferred embodiments of the present invention, there is provided an isolated chimeric polypeptide encoding for an edge portion of HUMLYSYL_PEA_{—}1_P7 (SEQ ID NO:373), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VL, having a structure as follows: a sequence starting from any of amino acid numbers 249−x to 249; and ending at any of amino acid numbers 250+((n−2)−x), in which x varies from 0 to n−2.

According to preferred embodiments of the present invention, there is provided an isolated chimeric polypeptide encoding for HUMLYSYL_PEA_{—}1_P13 (SEQ ID NO:374), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVETPCPDVYWFPIFTEVACDELVEEMEHFGQWSLG NNK corresponding to amino acids 1-585 of PLO1_HUMAN_V1 (SEQ ID NO:368), which also corresponds to amino acids 1-585 of HUMLYSYL_PEA_{—}1_P13 (SEQ ID NO:374), and a second amino acid sequence being at least 70%, optionally at least 80%, preferably at least 85%, more preferably at least 90% and most preferably at least 95% homologous to a polypeptide having the sequence GCPESGTSASMAGHESKP (SEQ ID NO:475) corresponding to amino acids 586-603 of HUMLYSYL_PEA_{—}1_P13 (SEQ ID NO:374), wherein said first amino acid sequence and second amino acid sequence are contiguous and in a sequential order.

According to preferred embodiments of the present invention, there is provided an isolated polypeptide encoding for a tail of HUMLYSYL_PEA_{—}1_P13 (SEQ ID NO:374), comprising a polypeptide being at least 70%, optionally at least about 80%, preferably at least about 85%, more preferably at least about 90% and most preferably at least about 95% homologous to the sequence GCPESGTSASMAGHESKP (SEQ ID NO:475) in HUMLYSYL_PEA_{—}1_P13 (SEQ ID NO:374).

According to preferred embodiments of the present invention, there is provided an isolated chimeric polypeptide encoding for HUMLYSYL_PEA_{—}1_P14 (SEQ ID NO:375), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVETPCPDVYWFPIFTEVACDELVEEMEHFGQWSLG NNK corresponding to amino acids 1-585 of PLO1_HUMAN_V1 (SEQ ID NO:368), which also corresponds to amino acids 1-585 of HUMLYSYL_PEA_{—}1_P14 (SEQ ID NO:375), and a second amino acid sequence being at least 70%, optionally at least 80%, preferably at least 85%, more preferably at least 90% and most preferably at least 95% homologous to a polypeptide having the sequence TATPENLLGDRRGICAQLDLLLACGEGSDRSTHHTGSPCPGCL (SEQ ID NO:476) corresponding to amino acids 586-628 of HUMLYSYL_PEA_{—}1_P14 (SEQ ID NO:375), wherein said first amino acid sequence and second amino acid sequence are contiguous and in a sequential order.

According to preferred embodiments of the present invention, there is provided an isolated polypeptide encoding for a tail of HUMLYSYL_PEA_{—}1_P14 (SEQ ID NO:375), comprising a polypeptide being at least 70%, optionally at least about 80%, preferably at least about 85%, more preferably at least about 90% and most preferably at least about 95% homologous to the sequence TATPENLLGDRRGICAQLDLLLACGEGSDRSTHHTGSPCPGCL (SEQ ID NO:476) in HUMLYSYL_PEA_{—}1_P14 (SEQ ID NO:375).

According to preferred embodiments of the present invention, there is provided an isolated chimeric polypeptide encoding for HUMLYSYL_PEA_{—}1_P16 (SEQ ID NO:376), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVET corresponding to amino acids 1-550 of PLO1_HUMAN_V1 (SEQ ID NO:368), which also corresponds to amino acids 1-550 of HUMLYSYL_PEA_{—}1_P16 (SEQ ID NO:376), and a second amino acid sequence being at least 70%, optionally at least 80%, preferably at least 85%, more preferably at least 90% and most preferably at least 95% homologous to a polypeptide having the sequence VRAMDTLLDQPCLLQGAGHRRETACPGEWGTAGWEL (SEQ ID NO:477) corresponding to amino acids 551-586 of HUMLYSYL_PEA_{—}1_P16 (SEQ ID NO:376), wherein said first amino acid sequence and second amino acid sequence are contiguous and in a sequential order.

According to preferred embodiments of the present invention, there is provided an isolated polypeptide encoding for a tail of HUMLYSYL_PEA_{—}1_P16 (SEQ ID NO:376), comprising a polypeptide being at least 70%, optionally at least about 80%, preferably at least about 85%, more preferably at least about 90% and most preferably at least about 95% homologous to the sequence VRAMDTLLDQPCLLQGAGHRRETACPGEWGTAGWEL (SEQ ID NO:477) in HUMLYSYL_PEA_{—}1_P16 (SEQ ID NO:376).

According to preferred embodiments of the present invention, there is provided an isolated chimeric polypeptide encoding for HUMLYSYL_PEA_{—}1_P24 (SEQ ID NO:378), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKR corresponding to amino acids 1-193 of PLO1_HUMAN_V1 (SEQ ID NO:368), which also corresponds to amino acids 1-193 of HUMLYSYL_PEA_{—}1_P24 (SEQ ID NO:378), and a second amino acid sequence being at least 70%, optionally at least 80%, preferably at least 85%, more preferably at least 90% and most preferably at least 95% homologous to a polypeptide having the sequence VSRLHS (SEQ ID NO:478) corresponding to amino acids 194-199 of HUMLYSYL_PEA_{—}1_P24 (SEQ ID NO:378), wherein said first amino acid sequence and second amino acid sequence are contiguous and in a sequential order.

According to preferred embodiments of the present invention, there is provided an isolated polypeptide encoding for a tail of HUMLYSYL_PEA_{—}1_P24 (SEQ ID NO:378), comprising a polypeptide being at least 70%, optionally at least about 80%, preferably at least about 85%, more preferably at least about 90% and most preferably at least about 95% homologous to the sequence VSRLHS (SEQ ID NO:478) in HUMLYSYL_PEA_{—}1_P24 (SEQ ID NO:378).

According to preferred embodiments of the present invention, there is provided an isolated chimeric polypeptide encoding for HUMICAMA1A_PEA_{—}1_P2 (SEQ ID NO:309), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTFVLPATPPQLVSPRVLEVDTQGTVV CSLDGLFPVSEAQVHLALGDQRLNPTVTYGNDSFSAKASVSVTAEDEGTQRLTCAVILG NQSQETLQTVTIYS corresponding to amino acids 1-309 of ICA1_HUMAN (SEQ ID NO:307), which also corresponds to amino acids 1-309 of HUMICAMA1A_PEA_{—}1_P2 (SEQ ID NO:309), and a second amino acid sequence being at least 70%, optionally at least 80%, preferably at least 85%, more preferably at least 90% and most preferably at least 95% homologous to a polypeptide having the sequence KKGQGRSGASWGCDLNPGRGSLCAYSRLSGAQRDSDEARGLRRDRGDSEV (SEQ ID NO:479) corresponding to amino acids 310-359 of HUMICAMA1A_PEA_{—}1_P2 (SEQ ID NO:309), wherein said first amino acid sequence and second amino acid sequence are contiguous and in a sequential order.

According to preferred embodiments of the present invention, there is provided an isolated polypeptide encoding for a tail of HUMICAMA1A_PEA_{—}1_P2 (SEQ ID NO:309), comprising a polypeptide being at least 70%, optionally at least about 80%, preferably at least about 85%, more preferably at least about 90% and most preferably at least about 95% homologous to the sequence KKGQGRSGASWGCDLNPGRGSLCAYSRLSGAQRDSDEARGLRRDRGDSEV (SEQ ID NO:479) in HUMICAMA1A_PEA_{—}1_P2 (SEQ ID NO:309).

According to preferred embodiments of the present invention, there is provided an isolated chimeric polypeptide encoding for HUMICAMA1A_PEA_{—}1_P5 (SEQ ID NO:310), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTFVLPATPPQLVSRVLEVDTQGTVVC SLDGLFPVSEAQVHLALGDQRLNPTVTYGNDSFSAKASVSVTAEDEGTQRLTCAVILGN QSQETLQTVTIYSFPAPNVILTKPEVSEGTEVTVKCEAHPRAKVTLNGVPAQPLGPRAQL LLKATPEDNGRSFSCSATLEVAGQLIHKNQTRELRVL corresponding to amino acids 1-393 of ICA1_HUMAN (SEQ ID NO:307), which also corresponds to amino acids 1-393 of HUMICAMA1A_PEA_{—}1_P5 (SEQ ID NO:310), and a second amino acid sequence being at least 70%, optionally at least 80%, preferably at least 85%, more preferably at least 90% and most preferably at least 95% homologous to a polypeptide having the sequence CEWGCWSMAPIPQGPISLKVP (SEQ ID NO:480) corresponding to amino acids 394-414 of HUMICAMA1A_PEA_{—}1_P5 (SEQ ID NO:310), wherein said first amino acid sequence and second amino acid sequence are contiguous and in a sequential order.

According to preferred embodiments of the present invention, there is provided an isolated polypeptide encoding for a tail of HUMICAMA1A_PEA_{—}1_P5 (SEQ ID NO:310), comprising a polypeptide being at least 70%, optionally at least about 80%, preferably at least about 85%, more preferably at least about 90% and most preferably at least about 95% homologous to the sequence CEWGCWSMAPIPQGPISLKVP (SEQ ID NO:480) in HUMICAMA1A_PEA_{—}1_P5 (SEQ ID NO:310).

According to preferred embodiments of the present invention, there is provided an isolated chimeric polypeptide encoding for HUMICAMA1A_PEA_{—}1_P8 (SEQ ID NO:311), comprising a first amino acid sequence being at least 90% homologous to MAPSSPRPALPALLVLLGALFPG corresponding to amino acids 1-23 of ICA1_HUMAN_V1 (SEQ ID NO:308), which also corresponds to amino acids 1-23 of HUMICAMA1A_PEA_L_P8 (SEQ ID NO:311), and a second amino acid sequence being at least 90% homologous to TPERVELAPLPSWQPVGKNLTLRCQVEGGAPRANLTVVLLRGEKELKREPAVGEPAEV TTTVLVRRDHHGANFSCRTELDLRPQGLELFENTSAPYQLQTFVLPATPPQLVSPRVLE VDTQGTVVCSLDGLFPVSEAQVHLALGDQRLNPTVTYGNDSFSAKASVSVTAEDEGTQ RLTCAVILGNQSQETLQTVTIYSFPAPNVILTKPEVSEGTEVTVKCEAHPRAKVTLNGVP AQPLGPRAQLLLKATPEDNGRSFSCSATLEVAGQLIHKNQTRELRVLYGPRLDERDCPG NWTWPENSQQTPMCQAWGNPLPELKCLKDGTFPLPIGESVTVTRDLEGTYLCRARSTQ GEVTRKVTVNVLSPRYEIVIITVVAAAVIMGTAGLSTYLYNRQRKIKKYRLQQAQKGTP MKPNTQATPP corresponding to amino acids 112-532 of ICA1_HUMAN_V1 (SEQ ID NO:308), which also corresponds to amino acids 24-444 of HUMICAMA1A_PEA_{—}1_P8 (SEQ ID NO:311), wherein said first amino acid sequence and second amino acid sequence are contiguous and in a sequential order.

According to preferred embodiments of the present invention, there is provided an isolated chimeric polypeptide encoding for an edge portion of HUMICAMA1A_PEA_{—}1_P8 (SEQ ID NO:311), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GT, having a structure as follows: a sequence starting from any of amino acid numbers 23−x to 23; and ending at any of amino acid numbers 24+((n−2)−x), in which x varies from 0 to n−2.

According to preferred embodiments of the present invention, there is provided an isolated chimeric polypeptide encoding for HUMICAMA1A_PEA_{—}1_P15 (SEQ ID NO:312), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTF corresponding to amino acids 1-212 of ICA1_HUMAN (SEQ ID NO:307), which also corresponds to amino acids 1-212 of HUMICAMA1A_PEA_{—}1_P15 (SEQ ID NO:312), and a second amino acid sequence being at least 70%, optionally at least 80%, preferably at least 85%, more preferably at least 90% and most preferably at least 95% homologous to a polypeptide having the sequence GED corresponding to amino acids 213-215 of HUMICAMA1A_PEA_{—}1_P15 (SEQ ID NO:312), wherein said first amino acid sequence and second amino acid sequence are contiguous and in a sequential order.

According to preferred embodiments of the present invention, there is provided an isolated chimeric polypeptide encoding for HUM4COLA_PEA_{—}1_P7 (SEQ ID NO:276), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGVVVPTRFGNADGAACHF PFIFEGRSYSACTTDGRSDGLPWCSTTANYDTDDRFGFCPSERLYTRDGNADGKPCQFP FIFQGQSYSACTTDGRSDGYRWCATTANYDRDKLFGFCPTRADSTVMGGNSAGELCVF PFTFLGKE corresponding to amino acids 1-357 of MM09_HUMAN (SEQ ID NO:275), which also corresponds to amino acids 1-357 of HUM4COLA_PEA_{—}1_P7 (SEQ ID NO:276), and a second amino acid sequence being at least 70%, optionally at least 80%, preferably at least 85%, more preferably at least 90% and most preferably at least 95% homologous to a polypeptide having the sequence SSP (SEQ ID NO:481) corresponding to amino acids 358-360 of HUM4COLA_PEA_{—}1_P7 (SEQ ID NO:276), wherein said first amino acid sequence and second amino acid sequence are contiguous and in a sequential order.

According to preferred embodiments of the present invention, there is provided an isolated polypeptide encoding for a tail of HUM4COLA-PEA_{—}1_P7 (SEQ ID NO:276), comprising a polypeptide being at least 70%, optionally at least about 80%, preferably at least about 85%, more preferably at least about 90% and most preferably at least about 95% homologous to the sequence SSP (SEQ ID NO:481) in HUM4COLA_PEA_{—}1_P7 (SEQ ID NO:276).

According to preferred embodiments of the present invention, there is provided an isolated chimeric polypeptide encoding for HUM4COLA_PEA_{—}1_P14 (SEQ ID NO:277), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGVVVPTRFGNADGAACHF PFIFEGRSYSACTTDGRSDGLPWCSTTANYDTDDRFGFCPSE corresponding to amino acids 1-274 of MM09_HUMAN (SEQ ID NO:275), which also corresponds to amino acids 1-274 of HUM4COLA_PEA_{—}1_P14 (SEQ ID NO:277), and a second amino acid sequence being at least 70%, optionally at least 80%, preferably at least 85%, more preferably at least 90% and most preferably at least 95% homologous to a polypeptide having the sequence SE corresponding to amino acids 275-276 of HUM4COLA_PEA_{—}1_P14 (SEQ ID NO:277), wherein said first amino acid sequence and second amino acid sequence are contiguous and in a sequential order.

According to preferred embodiments of the present invention, there is provided an isolated chimeric polypeptide encoding for HUM4COLA_PEA_{—}1_P15 (SEQ ID NO:278), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGV corresponding to amino acids 1-216 of MM09_HUMAN (SEQ ID NO:275), which also corresponds to amino acids 1-216 of HUM4COLA_PEA_{—}1_P15 (SEQ ID NO:278), and a second amino acid sequence being at least 70%, optionally at least 80%, preferably at least 85%, more preferably at least 90% and most preferably at least 95% homologous to a polypeptide having the sequence GEILSPPGP (SEQ ID NO:482) corresponding to amino acids 217-225 of HUM4COLA_PEA_{—}1_P15 (SEQ ID NO:278), wherein said first amino acid sequence and second amino acid sequence are contiguous and in a sequential order.

According to preferred embodiments of the present invention, there is provided an isolated polypeptide encoding for a tail of HUM4COLA_PEA_{—}1_P15 (SEQ ID NO:278), comprising a polypeptide being at least 70%, optionally at least about 80%, preferably at least about 85%, more preferably at least about 90% and most preferably at least about 95% homologous to the sequence GEILSPPGP (SEQ ID NO:482) in HUM4COLA_PEA_{—}1_P15 (SEQ ID NO:278).

According to preferred embodiments of the present invention, there is provided an isolated chimeric polypeptide encoding for HSSTROMR_PEA_{—}1_P4 (SEQ ID NO:244), comprising a first amino acid sequence being at least 90% homologous to MKSLPILLLLCVAVCSAYPLDGAARGEDTSMNLV corresponding to amino acids 1-34 of MM03_HUMAN (SEQ ID NO:243), which also corresponds to amino acids 1-34 of HSSTROMR_PEA_{—}1_P4 (SEQ ID NO:244), and a second amino acid sequence being at least 90% homologous to QKFLGLEVTGKLDSDTLEVMRKPRCGVPDVGHFRTFPGIPKWRKTHLTYRIVNYTPDLP KDAVDSAVEKALKVWEEVTPLTFSRLYEGEADIMISFAVREHGDFYPFDGPGNVLAHA YAPGPGINGDAHFDDDEQWTKDTTGTNLFLVAAHEIGHSLGLFHSANTEALMYPLYHS LTDLTRFRLSQDDINGIQSLYGPPPDSPETPLVPTEPVPPEPGTPANCDPALSFDAVSTLR GEILIFKDRHFWRKSLRKLEPELHLISSFWPSLPSGVDAAYEVTSKDLVFIFKGNQFWAIR GNEVRAGYPRGIHTLGFPPTVRKIDAAISDKEKNKTYFFVEDKYWRFDEKRNSMEPGFP KQIAEDFPGIDSKIDAVFEEFGFFYFFTGSSQLEFDPNAKKVTHTLKSNSWLNC corresponding to amino acids 68-477 of MM03_HUMAN (SEQ ID NO:243), which also corresponds to amino acids 35-444 of HSSTROMR_PEA_{—}1_P4 (SEQ ID NO:244), wherein said first amino acid sequence and second amino acid sequence are contiguous and in a sequential order.

According to preferred embodiments of the present invention, there is provided an isolated chimeric polypeptide encoding for an edge portion of HSSTROMR_PEA_{—}1_P4 (SEQ ID NO:244), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VQ, having a structure as follows: a sequence starting from any of amino acid numbers 34−x to 34; and ending at any of amino acid numbers 35+((n−2)−x), in which x varies from 0 to n−2.

According to preferred embodiments of the present invention, there is provided an isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPTVK (SEQ ID NO:483) corresponding to amino acids 1-7 of HSIGFACI_PEA_{—}1_P5 (SEQ ID NO:225), a second amino acid sequence being at least 90% homologous to MHTMSSSHLFYLALCLLTFTSSATAGPETLCGAELVDALQFVCGDRGFYFNKPTGYGSS SRRAPQTGIVDECCFRSCDLRRLEMYCAPLKPAKSARSVRAQRHTDMPKTQK corresponding to amino acids 1-111 of Q9NP10 (SEQ ID NO:222), which also corresponds to amino acids 8-118 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPTVK (SEQ ID NO:483) of HSIGFACI_PEA_{—}1_P5 (SEQ ID NO:225).

According to preferred embodiments of the present invention, there is provided an isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

According to preferred embodiments of the present invention, there is provided an isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), and a second amino acid sequence being at least 90% homologous to VKMHTMSSSHLFYLALCLLTFTS SATAGPETLCGAELVDALQFVCGDRGFYFNKPTGY GSSSRRAPQTGIVDECCFRSCDLRRLEMYCAPLKPAKSARSVRAQRHTDMPKTQKYQP PSTNKNTKSQRRKGSTFEERK corresponding to amino acids 3-139 of Q13429 (SEQ ID NO:224), which also corresponds to amino acids 6-142 of HSIGFACI_PEA_{—}1_P5 (SEQ ID NO:225), wherein said first amino acid sequence and second amino acid sequence are contiguous and in a sequential order.

According to preferred embodiments of the present invention, there is provided an isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

According to preferred embodiments of the present invention, there is provided an isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQKYQP PSTNKNTKSQRRKG corresponding to amino acids 22-151 of IGFB_HUMAN (SEQ ID NO:220), which also corresponds to amino acids 6-135 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence STFEERK corresponding to amino acids 136-142 of HSIGFACI_PEA_{—}1_P5 (SEQ ID NO:225), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

According to preferred embodiments of the present invention, there is provided an isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence STFEERK in HSIGFACI_PEA_{—}1_P5 (SEQ ID NO:225).

According to preferred embodiments of the present invention, there is provided an isolated chimeric polypeptide encoding for HSIGFACI_PEA_{—}1_P5 (SEQ ID NO:225), comprising a first amino acid sequence being at least 90% homologous to MITPTVKMHTMSSSHLFYLALCLLTFTSSATAGPETLCGAELVDALQFVCGDRGFYFNK PTGYGSSSRRAPQTGIVDECCFRSCDLRRLEMYCAPLKPAKSARSVRAQRHTDMPKTQ K corresponding to amino acids 1-118 of Q14620 (SEQ ID NO:221), which also corresponds to amino acids 1-118 of HSIGFACI_PEA_{—}1_P5 (SEQ ID NO:225), and a second amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

According to preferred embodiments of the present invention, there is provided an isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQK corresponding to amino acids 22-134 of IGFA_HUMAN (SEQ ID NO:223), which also corresponds to amino acids 6-118 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

According to preferred embodiments of the present invention, there is provided an isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

According to preferred embodiments of the present invention, there is provided an isolated chimeric polypeptide encoding for HSIGFACI_PEA_{—}1_P2 (SEQ ID NO:226), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P2 (SEQ ID NO:226), and a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQKEVH LKNASRGSAGNKNYRM (SEQ ID NO:487) corresponding to amino acids 22-153 of IGFA_HUMAN (SEQ ID NO:223), which also corresponds to amino acids 6-137 of HSIGFACI_PEA_{—}1_P2 (SEQ ID NO:226), wherein said first amino acid sequence and second amino acid sequence are contiguous and in a sequential order.

According to preferred embodiments of the present invention, there is provided an isolated polypeptide encoding for a head of HSIGFACI_PEA_{—}1_P2 (SEQ ID NO:226), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P2 (SEQ ID NO:226).

According to preferred embodiments of the present invention, there is provided an isolated chimeric polypeptide encoding for HSIGFACI_PEA_{—}1_P6 (SEQ ID NO: 227), comprising a first amino acid sequence being at least 90% homologous to MGKISSLPTQLFKCCFCDFLKVKMHTMSSSHLFYLALCLLTFTSSATAGPETLCGAELV DALQFVCGDRGFYFNKPTGYGSSSRRAPQTGIVDECCFRSCDLRRLEMYCAPLKPAKS ARSVRAQRHTDMPKTQK corresponding to amino acids 1-134 of IGFA_HUMAN (SEQ ID NO:223), which also corresponds to amino acids 1-134 of HSIGFACI_PEA_{—}1_P6 (SEQ ID NO: 227), and a second amino acid sequence being at least 70%, optionally at least 80%, preferably at least 85%, more preferably at least 90% and most preferably at least 95% homologous to a polypeptide having the sequence YQPPSTNKNTKSQRRKGWPKTHPGGEQKEGTEASLQIRGKKKEQRREIGSRNAECRGK KGK (SEQ ID NO:486) corresponding to amino acids 135-195 of HSIGFACI_PEA_{—}1_P6 (SEQ ID NO: 227), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P6 (SEQ ID NO: 227), comprising a polypeptide being at least 70%, optionally at least about 80%, preferably at least about 85%, more preferably at least about 90% and most preferably at least about 95% homologous to the sequence YQPPSTNKNTKSQRRKGWPKTHPGGEQKEGTEASLQIRGKKKEQRREIGSRNAECRGK KGK (SEQ ID NO:486) in HSIGFACI_PEA_{—}1_P6 (SEQ ID NO: 227).

According to preferred embodiments of the present invention, there is provided an isolated chimeric polypeptide encoding for HSIGFACI_PEA_{—}1_P1 (SEQ ID NO:228), comprising a first amino acid sequence being at least 90% homologous to MGKISSLPTQLFKCCFCDFLKVKMHTMSSSHLFYLALCLLTFTSSATAGPETLCGAELV DALQFVCGDRGFYFNKPTGYGSSSRRAPQTGIVDECCFRSCDLRRLEMYCAPLKPAKS ARSVRAQRHTDMPKTQK corresponding to amino acids 1-134 of IGFB_HUMAN (SEQ ID NO:220), which also corresponds to amino acids 1-134 of HSIGFACI_PEA_{—}1_P1 (SEQ ID NO:228), and a second amino acid sequence being at least 70%, optionally at least 80%, preferably at least 85%, more preferably at least 90% and most preferably at least 95% homologous to a polypeptide having the sequence EVHLKNASRGSAGNKNYRM (SEQ ID NO:487) corresponding to amino acids 135-153 of HSIGFACI_PEA_{—}1_P1 (SEQ ID NO:228), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P1 (SEQ ID NO:228), comprising a polypeptide being at least 70%, optionally at least about 80%, preferably at least about 85%, more preferably at least about 90% and most preferably at least about 95% homologous to the sequence EVHLKNASRGSAGNKNYRM (SEQ ID NO:487) in HSIGFACI_PEA_{—}1_P1 (SEQ ID NO:228).

According to preferred embodiments of the present invention, there is provided an isolated chimeric polypeptide encoding for HSIGFACI_PEA_{—}1_P7 (SEQ ID NO:229), comprising a first amino acid sequence being at least 90% homologous to MGKISSLPTQLFKCCFCDFLKVKMHTMSSSHLFYLALCLLTFTSSATAGPETLCGAELV DALQFVCGDRGFYF corresponding to amino acids 1-73 of IGFB_HUMAN (SEQ ID NO:220), which also corresponds to amino acids 1-73 of HSIGFACI_PEA_{—}1_P7 (SEQ ID NO:229), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 74-108 of HSIGFACI_PEA_{—}1_P7 (SEQ ID NO:229), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P7 (SEQ ID NO:229), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P7 (SEQ ID NO:229).

According to preferred embodiments of the present invention, there is provided an isolated chimeric polypeptide encoding for HSIGFACI_PEA_{—}1_P7 (SEQ ID NO:229), comprising a first amino acid sequence being at least 90% homologous to MGKISSLPTQLFKCCFCDFLKVKMHTMSSSHLFYLALCLLTFTS SATAGPETLCGAELV DALQFVCGDRGFYF corresponding to amino acids 1-73 of IGFA_HUMAN (SEQ ID NO:223), which also corresponds to amino acids 1-73 of HSIGFACI_PEA_{—}1_P7 (SEQ ID NO:229), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 74-108 of HSIGFACI_PEA_{—}1_P7 (SEQ ID NO:229), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P7 (SEQ ID NO:229), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P7 (SEQ ID NO:229).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPTVK (SEQ ID NO:483) corresponding to amino acids 1-7 of HSIGFACI_PEA_{—}1_P8 (SEQ ID NO:230), a second amino acid sequence being at least 90% homologous to MHTMSSSHLFYLALCLLTFTSSATAGPETLCGAELVDALQFVCGDRGFYF corresponding to amino acids 1-50 of Q9NP10 (SEQ ID NO:222), which also corresponds to amino acids 8-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPTVK (SEQ ID NO:483) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 3-54 of Q13429 (SEQ ID NO:224), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 90% homologous to MITPTVKMHTMSSSHLFYLALCLLTFTSSATAGPETLCGAELVDALQFVCGDRGFYF corresponding to amino acids 1-57 of Q14620 (SEQ ID NO:221), which also corresponds to amino acids 1-57 of HSIGFACI_PEA_{—}1_P8 (SEQ ID NO:230), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFB_HUMAN (SEQ ID NO:220), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFA_HUMAN (SEQ ID NO:223), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 3-54 of Q13429 (SEQ ID NO:224), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 90% homologous to MITPTVKMHTMSSSHLFYLALCLLTFTSSATAGPETLCGAELVDALQFVCGDRGFYF corresponding to amino acids 1-57 of Q14620 (SEQ ID NO:221), which also corresponds to amino acids 1-57 of HSIGFACI_PEA_{—}1_P8 (SEQ ID NO:230), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence and second amino acid sequence are contiguous and in a sequential order.

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFB_HUMAN (SEQ ID NO:220), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFA_HUMAN (SEQ ID NO:223), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

According to preferred embodiments of the present invention, there is provided an isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

According to preferred embodiments of the present invention, there is provided an isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

According to preferred embodiments of the present invention, there is provided an isolated chimeric polypeptide encoding for S56892_PEA_{—}1_P2 (SEQ ID NO:194), comprising a first amino acid sequence being at least 70%, optionally at least 80%, preferably at least 85%, more preferably at least 90% and most preferably at least 95% homologous to a polypeptide having the sequence MNSFSTSKCRKSLALELPAAVEPCVREGCVAQGGLAGGQQQRQAPSCAVSSPLRSLPS GTG (SEQ ID NO:491) corresponding to amino acids 1-61 of S56892_PEA_{—}1_P2 (SEQ ID NO:194), and a second amino acid sequence being at least 90% homologous to AFGPVAFSLGLLLVLPAAFPAPVPPGEDSKDVAAPHRQPLTSSERIDKQIRYILDGISALR KETCNKSNMCESSKEALAENNLNLPKMAEKDGCFQSGFNEETCLVKIITGLLEFEVYLE YLQNRFESSEEQARAVQMSTKVLIQFLQKKAKNLDAITTPDPTTNASLLTKLQAQNQW LQDMTTHLILRSFKEFLQSSLRALRQM corresponding to amino acids 8-212 of IL6_HUMAN (SEQ ID NO:193), which also corresponds to amino acids 62-266 of S56892_PEA_{—}1_P2 (SEQ ID NO:194), wherein said first amino acid sequence and second amino acid sequence are contiguous and in a sequential order.

According to preferred embodiments of the present invention, there is provided an isolated polypeptide encoding for a head of S56892_PEA_{—}1_P2 (SEQ ID NO:194), comprising a polypeptide being at least 70%, optionally at least about 80%, preferably at least about 85%, more preferably at least about 90% and most preferably at least about 95% homologous to the sequence MNSFSTSKCRKSLALELPAAVEPCVREGCVAQGGLAGGQQQRQAPSCAVSSPLRSLPS GTG (SEQ ID NO:491) of S56892_PEA_{—}1_P2 (SEQ ID NO:194).

According to preferred embodiments of the present invention, there is provided an isolated chimeric polypeptide encoding for S56892_PEA_{—}1_P8 (SEQ ID NO:195), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSNMCESSKEALAENNLNLPKMAEKDGCFQSGFNEETCLVKIITGLL EFEVYLEYLQNRFESSEEQARAVQMSTKVLIQFLQKK corresponding to amino acids 1-157 of IL6_HUMAN (SEQ ID NO:193), which also corresponds to amino acids 1-157 of S56892_PEA_{—}1_P8 (SEQ ID NO:195), and a second amino acid sequence being at least 70%, optionally at least 80%, preferably at least 85%, more preferably at least 90% and most preferably at least 95% homologous to a polypeptide having the sequence VGVSSFPQLGVGEDRLKDSVLDNSGMQCHFQKRRLHVNKRV (SEQ ID NO:492) corresponding to amino acids 158-198 of S56892-PEA_{—}1_P8 (SEQ ID NO:195), wherein said first amino acid sequence and second amino acid sequence are contiguous and in a sequential order.

According to preferred embodiments of the present invention, there is provided an isolated polypeptide encoding for a tail of S56892_PEA_{—}1_P8 (SEQ ID NO:195), comprising a polypeptide being at least 70%, optionally at least about 80%, preferably at least about 85%, more preferably at least about 90% and most preferably at least about 95% homologous to the sequence VGVSSFPQLGVGEDRLKDSVLDNSGMQCHFQKRRLHVNKRV (SEQ ID NO:492) in S56892_PEA_{—}1_P8 (SEQ ID NO:195).

According to preferred embodiments of the present invention, there is provided an isolated chimeric polypeptide encoding for S56892_PEA_{—}1_P9 (SEQ ID NO:196), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSNMCESSKEALAENNLNLPKMAEKDGCFQSGFNE corresponding to amino acids 1-108 of IL6_HUMAN (SEQ ID NO:193), which also corresponds to amino acids 1-108 of S56892_PEA_{—}1_P9 (SEQ ID NO:196), and a second amino acid sequence being at least 90% homologous to AKNLDAITTPDPTTNASLLTKLQAQNQWLQDMTTHLILRSFKEFLQSSLRALRQM corresponding to amino acids 158-212 of IL6_HUMAN (SEQ ID NO:193), which also corresponds to amino acids 109-163 of S56892_PEA_{—}1_P9 (SEQ ID NO:196), wherein said first amino acid sequence and second amino acid sequence are contiguous and in a sequential order.

According to preferred embodiments of the present invention, there is provided an isolated chimeric polypeptide encoding for an edge portion of S56892_PEA_{—}1_P9 (SEQ ID NO:196), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EA, having a structure as follows: a sequence starting from any of amino acid numbers 108−x to 108; and ending at any of amino acid numbers 109+((n−2)−x), in which x varies from 0 to n−2.

According to preferred embodiments of the present invention, there is provided an isolated chimeric polypeptide encoding for S56892_PEA_{—}1_P11 (SEQ ID NO:197), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSN corresponding to amino acids 1-76 of IL6_HUMAN (SEQ ID NO:193), which also corresponds to amino acids 1-76 of S56892_PEA_{—}1_P11 (SEQ ID NO:197), and a second amino acid sequence being at least 70%, optionally at least 80%, preferably at least 85%, more preferably at least 90% and most preferably at least 95% homologous to a polypeptide having the sequence IWLKKMDASNLDSMRRLAW (SEQ ID NO:493) corresponding to amino acids 77-95 of S56892_PEA_{—}1_P11 (SEQ ID NO:197), wherein said first amino acid sequence and second amino acid sequence are contiguous and in a sequential order.

According to preferred embodiments of the present invention, there is provided an isolated polypeptide encoding for a tail of S56892_PEA_{—}1_P11 (SEQ ID NO:197), comprising a polypeptide being at least 70%, optionally at least about 80%, preferably at least about 85%, more preferably at least about 90% and most preferably at least about 95% homologous to the sequence IWLKKMDASNLDSMRRLAW (SEQ ID NO:493) in S56892_PEA_{—}1_P11 (SEQ ID NO:197).

According to preferred embodiments of the present invention, there is provided an isolated chimeric polypeptide encoding for HSHGFR_P6 (SEQ ID NO:165), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEHSFLPSSYRGKDLQENYCR NPRGEEGGPWCFTSNPEVRYEVCDIPQCSEVECMTCNGESYRGLMDHTESGKICQRWD HQTPHRHKFLPERYPDKGFDDNYCRNPDGQPRPWCYTLDPHTRWEYCAIKTCA corresponding to amino acids 1-289 of HGF_HUMAN (SEQ ID NO:164), which also corresponds to amino acids 1-289 of HSHGFR_P6 (SEQ ID NO:165), and a second amino acid sequence being at least 70%, optionally at least 80%, preferably at least 85%, more preferably at least 90% and most preferably at least 95% homologous to a polypeptide having the sequence E corresponding to amino acids 290-290 of HSHGFR_P6 (SEQ ID NO:165), wherein said first amino acid sequence and second amino acid sequence are contiguous and in a sequential order.

According to preferred embodiments of the present invention, there is provided an isolated chimeric polypeptide encoding for HSHGFR_P11 (SEQ ID NO:166), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEH corresponding to amino acids 1-160 of HGF_HUMAN (SEQ ID NO:164), which also corresponds to amino acids 1-160 of HSHGFR_P11 (SEQ ID NO:166), a second amino acid sequence being at least 90% homologous to SYRGKDLQENYCRNPRGEEGGPWCFTSNPEVRYEVCDIPQCSE corresponding to amino acids 166-208 of HGF_HUMAN (SEQ ID NO:164), which also corresponds to amino acids 161-203 of HSHGFR_P11 (SEQ ID NO:166), and a third amino acid sequence being at least 70%, optionally at least 80%, preferably at least 85%, more preferably at least 90% and most preferably at least 95% homologous to a polypeptide having the sequence GK corresponding to amino acids 204-205 of HSHGFR_P11 (SEQ ID NO:166), wherein said first amino acid sequence, second amino acid sequence and third amino acid sequence are contiguous and in a sequential order.

According to preferred embodiments of the present invention, there is provided an isolated chimeric polypeptide encoding for an edge portion of HSHGFR_P11 (SEQ ID NO:166), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HS, having a structure as follows: a sequence starting from any of amino acid numbers 160−x to 160; and ending at any of amino acid numbers 161+((n−2)−x), in which x varies from 0 to n−2.

According to preferred embodiments of the present invention, there is provided an isolated chimeric polypeptide encoding for HSHGFR_P12 (SEQ ID NO:167), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEH corresponding to amino acids 1-160 of HGF_HUMAN (SEQ ID NO:164), which also corresponds to amino acids 1-160 of HSHGFR_P12 (SEQ ID NO:167), and a second amino acid sequence being at least 70%, optionally at least 80%, preferably at least 85%, more preferably at least 90% and most preferably at least 95% homologous to a polypeptide having the sequence R corresponding to amino acids 161-161 of HSHGFR_P12 (SEQ ID NO:167), wherein said first amino acid sequence and second amino acid sequence are contiguous and in a sequential order.

According to preferred embodiments of the present invention, there is provided an isolated chimeric polypeptide encoding for HSHGFR_P13 (SEQ ID NO:168), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEHSFLPSSYRGKDLQENYCR NPRGEEGGPWCFTSNPEVRYEVCDIPQCSEVECMTCNGESYRGLMDHTESGKICQRWD HQTPHRHKFLPERYPDKGFDDNYCRNPDGQPRPWCYTLDPHTRWEYCAIK corresponding to amino acids 1-286 of HGF_HUMAN (SEQ ID NO:164), which also corresponds to amino acids 1-286 of HSHGFR_P13 (SEQ ID NO:168), and a second amino acid sequence being at least 70%, optionally at least 80%, preferably at least 85%, more preferably at least 90% and most preferably at least 95% homologous to a polypeptide having the sequence NMRDITWALN (SEQ ID NO:494) corresponding to amino acids 287-296 of HSHGFR_P13 (SEQ ID NO:168), wherein said first amino acid sequence and second amino acid sequence are contiguous and in a sequential order.

According to preferred embodiments of the present invention, there is provided an isolated polypeptide encoding for a tail of HSHGFR_P13 (SEQ ID NO:168), comprising a polypeptide being at least 70%, optionally at least about 80%, preferably at least about 85%, more preferably at least about 90% and most preferably at least about 95% homologous to the sequence NMRDITWALN (SEQ ID NO:494) in HSHGFR_P13 (SEQ ID NO:168).

According to preferred embodiments of the present invention, there is provided an isolated chimeric polypeptide encoding for HUMHPA1B_PEA_{—}1_P61 (SEQ ID NO:133), comprising a first amino acid sequence being at least 90% homologous to MSALGAVIALLLWGQLFAVDSGNDVTDI corresponding to amino acids 1-28 of HPT_HUMAN (SEQ ID NO:131), which also corresponds to amino acids 1-28 of HUMHPA1B_PEA_{—}1_P61 (SEQ ID NO:133), and a second amino acid sequence being at least 90% homologous to ADDGCPKPPEIAHGYVEHSVRYQCKNYYKLRTEGDGVYTLNNEKQWINKAVGDKLPE CEAVCGKPKNPANPVQRILGGHLDAKGSFPWQAKMVSHHNLTTGATLINEQWLLTTA KNLFLNHSENATAKDIAPTLTLYVGKKQLVEIEKVVLHPNYSQVDIGLIKLKQKVSVNE RVMPICLPSKDYAEVGRVGYVSGWGRNANFKFTDHLKYVMLPVADQDQCIRHYEGST VPEKKTPKSPVGVQPILNEHTFCAGMSKYQEDTCYGDAGSAFAVHDLEEDTWYATGIL SFDKSCAVAEYGVYVKVTSIQDWVQKTIAEN corresponding to amino acids 88-406 of HPT_HUMAN (SEQ ID NO:131), which also corresponds to amino acids 29-347 of HUMHPA1B_PEA_{—}1_P61 (SEQ ID NO:133), wherein said first amino acid sequence and second amino acid sequence are contiguous and in a sequential order.

According to preferred embodiments of the present invention, there is provided an isolated chimeric polypeptide encoding for an edge portion of HUMHPA1B_PEA_{—}1_P61 (SEQ ID NO:133), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IA, having a structure as follows: a sequence starting from any of amino acid numbers 28−x to 28; and ending at any of amino acid numbers 29+((n−2)−x), in which x varies from 0 to n−2.

According to preferred embodiments of the present invention, there is provided an isolated chimeric polypeptide encoding for HUMHPA1B_PEA_{—}1_P62 (SEQ ID NO:134), comprising a first amino acid sequence being at least 90% homologous to MSALGAVIALLLWGQLFAVDSGNDVTDIADDGCPKPPEIAHGYVEHSVRYQCKNYYK LRTEGDG corresponding to amino acids 1-64 of HPT_HUMAN (SEQ ID NO:131), which also corresponds to amino acids 1-64 of HUMHPA1B_PEA_{—}1_P62 (SEQ ID NO:134), and a second amino acid sequence being at least 70%, optionally at least 80%, preferably at least 85%, more preferably at least 90% and most preferably at least 95% homologous to a polypeptide having the sequence KMWTTVSMPYIQPPSLTFP (SEQ ID NO:495) corresponding to amino acids 65-83 of HUMHPA1B_PEA_{—}1_P62 (SEQ ID NO:134), wherein said first amino acid sequence and second amino acid sequence are contiguous and in a sequential order.

According to preferred embodiments of the present invention, there is provided an isolated polypeptide encoding for a tail of HUMHPA1B_PEA_{—}1_P62 (SEQ ID NO:134), comprising a polypeptide being at least 70%, optionally at least about 80%, preferably at least about 85%, more preferably at least about 90% and most preferably at least about 95% homologous to the sequence KMWTTVSMPYIQPPSLTFP (SEQ ID NO:495) in HUMHPA1B_PEA_{—}1_P62 (SEQ ID NO:134).

According to preferred embodiments of the present invention, there is provided an isolated chimeric polypeptide encoding for HUMHPA1B_PEA_{—}1_P64 (SEQ ID NO:135), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWfNKAVGDKLPECEADDGCPKPPEIAHGYVEHSVRYQCKNY YKLRTEGDG corresponding to amino acids 1-123 of HPT_HUMAN (SEQ ID NO:131), which also corresponds to amino acids 1-123 of HUMHPA1B_PEA_{—}1_P64 (SEQ ID NO:135), and a second amino acid sequence being at least 70%, optionally at least 80%, preferably at least 85%, more preferably at least 90% and most preferably at least 95% homologous to a polypeptide having the sequence KMWTTVSMPYIQPPSLTFP (SEQ ID NO:495) corresponding to amino acids 124-142 of HUMHPA1B_PEA_{—}1_P64 (SEQ ID NO:135), wherein said first amino acid sequence and second amino acid sequence are contiguous and in a sequential order.

According to preferred embodiments of the present invention, there is provided an isolated polypeptide encoding for a tail of HUMEPA1B_PEA_{—}1_P64 (SEQ ID NO:135), comprising a polypeptide being at least 70%, optionally at least about 80%, preferably at least about 85%, more preferably at least about 90% and most preferably at least about 95% homologous to the sequence KMWTTVSMPYIQPPSLTFP (SEQ ID NO:495) in HUMHPA1B_PEA_{—}1_P64 (SEQ ID NO:135).

According to preferred embodiments of the present invention, there is provided an isolated chimeric polypeptide encoding for HUMHPA1B_PEA_{—}1_P65 (SEQ ID NO:136), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEADDGCPKPPEIAHGYVEHSVRYQCKNY YKLRTEGDGVYTLNNEKQWINKAVGDKLPECEA corresponding to amino acids 1-147 of HPT_HUMAN (SEQ ID NO:131), which also corresponds to amino acids 1-147 of HUMHPA1B_PEA_{—}1_P65 (SEQ ID NO:136), and a second amino acid sequence being at least 70%, optionally at least 80%, preferably at least 85%, more preferably at least 90% and most preferably at least 95% homologous to a polypeptide having the sequence GGC corresponding to amino acids 148-150 of HUMHPA1B_PEA_{—}1_P65 (SEQ ID NO:136), wherein said first amino acid sequence and second amino acid sequence are contiguous and in a sequential order.

According to preferred embodiments of the present invention, there is provided an isolated chimeric polypeptide encoding for HUMHPA1B_PEA_{—}1_P68 (SEQ ID NO:137), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDK corresponding to amino acids 1-71 of HPT_HUMAN (SEQ ID NO:131), which also corresponds to amino acids 1-71 of HUMHPA1B_PEA_{—}1_P68 (SEQ ID NO:137), and a second amino acid sequence being at least 90% homologous to KQWINKAVGDKLPECEAVCGKPKNPANPVQRILGGHLDAKGSFPWQAKMVSHHNLTT GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 131-406 of HPT_HUMAN (SEQ ID NO:131), which also corresponds to amino acids 72-347 of HUMHPA1B_PEA_{—}1P68 (SEQ ID NO:137), wherein said first amino acid sequence and second amino acid sequence are contiguous and in a sequential order.

According to preferred embodiments of the present invention, there is provided an isolated chimeric polypeptide encoding for an edge portion of HUMHPA1B_PEA_{—}1P68 (SEQ ID NO:137), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KK, having a structure as follows: a sequence starting from any of amino acid numbers 71−x to 71; and ending at any of amino acid numbers 72+((n−2)−x), in which x varies from 0 to n−2.

According to preferred embodiments of the present invention, there is provided an isolated chimeric polypeptide encoding for HUMHPA1B_PEA_{—}1_P72 (SEQ ID NO:138), comprising a first amino acid sequence being at least 90% homologous to MSALGAVIALLLWGQLFAVDSGNDVTDIADDGCPKPPEIAHGYVEHSVRYQCKNYYK LRTEGD corresponding to amino acids 1-63 of HPT_HUMAN (SEQ ID NO:131), which also corresponds to amino acids 1-63 of HUMHPA1B_PEA_{—}1_P72 (SEQ ID NO:138), and a second amino acid sequence being at least 70%, optionally at least 80%, preferably at least 85%, more preferably at least 90% and most preferably at least 95% homologous to a polypeptide having the sequence ESGKPSAADPGWTPGCQRQLSLAG (SEQ ID NO:497) corresponding to amino acids 64-87 of HUMHPA1B_PEA_{—}1_P72 (SEQ ID NO:138), wherein said first amino acid sequence and second amino acid sequence are contiguous and in a sequential order.

According to preferred embodiments of the present invention, there is provided an isolated polypeptide encoding for a tail of HUMHPA1B_PEA_{—}1_P72 (SEQ ID NO:138), comprising a polypeptide being at least 70%, optionally at least about 80%, preferably at least about 85%, more preferably at least about 90% and most preferably at least about 95% homologous to the sequence ESGKPSAADPGWTPGCQRQLSLAG (SEQ ID NO:497) in HUMHPA1B_PEA_{—}1_P72 (SEQ ID NO:138).

According to preferred embodiments of the present invention, there is provided an isolated chimeric polypeptide encoding for HUMHPA1B_PEA_{—}1_P75 (SEQ ID NO:139), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEADDGCPKPPEIAHGYVEHSVRYQCKNY YKLRTEGDGVYTLNNEKQWINKAVGDKLPECEA corresponding to amino acids 1-147 of HPT_HUMAN (SEQ ID NO:131), which also corresponds to amino acids 1-147 of HUMHPA1B_PEA_{—}1_P75 (SEQ ID NO:139), and a second amino acid sequence being at least 90% homologous to GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 188-406 of HPT_HUMAN (SEQ ID NO:131), which also corresponds to amino acids 148-366 of HUMHPA1B_PEA_{—}1_P75 (SEQ ID NO:139), wherein said first amino acid sequence and second amino acid sequence are contiguous and in a sequential order.

According to preferred embodiments of the present invention, there is provided an isolated chimeric polypeptide encoding for an edge portion of HUMHPA1B_PEA_{—}1_P75 (SEQ ID NO:139), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 147−x to 147; and ending at any of amino acid numbers 148+((n−2)−x), in which x varies from 0 to n−2.

According to preferred embodiments of the present invention, there is provided an isolated chimeric polypeptide encoding for HUMHPA1B_PEA_{—}1_P76 (SEQ ID NO:140), comprising a first amino acid sequence being at least 90% homologous to MSALGAVIALLLWGQLFAVDSGNDVTDIADDGCPKPPEIAHGYVEHSVRYQ corresponding to amino acids 1-51 of HPT_HUMAN (SEQ ID NO:131), which also corresponds to amino acids 1-51 of HUMHPA1B_PEA_{—}1_P76 (SEQ ID NO:140), a second amino acid sequence bridging amino acid sequence comprising of L, and a third amino acid sequence being at least 90% homologous to QRILGGHLDAKGSFPWQAKMVSHHNLTTGATLINEQWLLTTAKNLFLNHSENATAKDI APTLTLYVGKKQLVEIEKVVLHPNYSQVDIGLIKLKQKVSVNERVMPICLPSKDYAEVG RVGYVSGWGRNANFKFTDHLKYVMLPVADQDQCIRHYEGSTVPEKKTPKSPVGVQPIL NEHTFCAGMSKYQEDTCYGDAGSAFAVHDLEEDTWYATGILSFDKSCAVAEYGVYVK VTSIQDWVQKTIAEN corresponding to amino acids 160-406 of HPT_HUMAN (SEQ ID NO:131), which also corresponds to amino acids 53-299 of HUMHPA1B_PEA_{—}1_P76 (SEQ ID NO:140), wherein said first amino acid sequence, second amino acid sequence and third amino acid sequence are contiguous and in a sequential order.

According to preferred embodiments of the present invention, there is provided an isolated polypeptide encoding for an edge portion of HUMHPA1B_PEA_{—}1_P76 (SEQ ID NO:140), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise QLQ having a structure as follows (numbering according to HUMHPA1B_PEA_{—}1_P76 (SEQ ID NO:140)): a sequence starting from any of amino acid numbers 51−x to 51; and ending at any of amino acid numbers 53+((n−2)−x), in which x varies from 0 to n−2.

According to preferred embodiments of the present invention, there is provided an isolated chimeric polypeptide encoding for HUMHPA1B_PEA_{—}1_P81 (SEQ ID NO:141), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEA corresponding to amino acids 1-88 of HPT_HUMAN (SEQ ID NO:131), which also corresponds to amino acids 1-88 of HUMHPA1B_PEA_{—}1_P81 (SEQ ID NO:141), and a second amino acid sequence being at least 90% homologous to GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 188-406 of HPT_HUMAN (SEQ ID NO:131), which also corresponds to amino acids 89-307 of HUMHPA1B_PEA_{—}1_P81 (SEQ ID NO:141), wherein said first amino acid sequence and second amino acid sequence are contiguous and in a sequential order.

According to preferred embodiments of the present invention, there is provided an isolated chimeric polypeptide encoding for an edge portion of HUMHPA1B_PEA_{—}1_P81 (SEQ ID NO:141), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 88−x to 88; and ending at any of amino acid numbers 89+((n−2)−x), in which x varies from 0 to n−2.

According to preferred embodiments of the present invention, there is provided an isolated chimeric polypeptide encoding for HUMBPA1B_PEA_{—}1_P83 (SEQ ID NO:142), comprising a first amino acid sequence being at least 90% homologous to MSALGAVIALLLWGQLFAVDSGNDVTDIAD corresponding to amino acids 1-30 of HPT_HUMAN (SEQ ID NO:131), which also corresponds to amino acids 1-30 of HUMHPA1B_PEA_{—}1_P83 (SEQ ID NO:142), and a second amino acid sequence being at least 70%, optionally at least 80%, preferably at least 85%, more preferably at least 90% and most preferably at least 95% homologous to a polypeptide having the sequence GFPP (SEQ ID NO:498) corresponding to amino acids 31-34 of HUMHPA1B_PEA_{—}1_P83 (SEQ ID NO:142), wherein said first amino acid sequence and second amino acid sequence are contiguous and in a sequential order.

According to preferred embodiments of the present invention, there is provided an isolated polypeptide encoding for a tail of HUMHPA1B_PEA_{—}1_P83 (SEQ ID NO:142), comprising a polypeptide being at least 70%, optionally at least about 80%, preferably at least about 85%, more preferably at least about 90% and most preferably at least about 95% homologous to the sequence GFPP (SEQ ID NO:498) in HUMHPA1B_PEA_{—}1_P83 (SEQ ID NO:142).

According to preferred embodiments of the present invention, there is provided an isolated chimeric polypeptide encoding for HUMHPA1B_PEA_{—}1_P106 (SEQ ID NO:143), comprising a first amino acid sequence being at least 90% homologous to MSALGAVIALLLWGQLFAVDSGNDVTDIADDGCPKPPEIAHGYVEHSVRYQCKNYYK LRTEGDGVYTLNN corresponding to amino acids 1-70 of HPT_HUMAN_V1 (SEQ ID NO:132), which also corresponds to amino acids 1-70 of HUMHPA1B_PEA_{—}1_P106 (SEQ ID NO:143), a bridging amino acid E corresponding to amino acid 71 of HUMHPA1B_PEA_{—}1_P106 (SEQ ID NO:143), a bridging amino acid E corresponding to amino acid 71 of HUMHPA1B_PEA_{—}1_P106 (SEQ ID NO:143), a second amino acid sequence being at least 90% homologous to KQWINKAVGDKLPECEA corresponding to amino acids 72-88 of HPT_HUMAN_V1 (SEQ ID NO:132), which also corresponds to amino acids 72-88 of HUMHPA1B_PEA_{—}1_P106 (SEQ ID NO:143), and a third amino acid sequence being at least 70%, optionally at least 80%, preferably at least 85%, more preferably at least 90% and most preferably at least 95% homologous to a polypeptide having the sequence AHTE (SEQ ID NO:499) corresponding to amino acids 89-92 of HUMHPA1B_PEA_{—}1_P106 (SEQ ID NO:143), wherein said first amino acid sequence, bridging amino acid, bridging amino acid, second amino acid sequence and third amino acid sequence are contiguous and in a sequential order.

According to preferred embodiments of the present invention, there is provided an isolated polypeptide encoding for a tail of HUMHPA1B_PEA_{—}1_P106 (SEQ ID NO:143), comprising a polypeptide being at least 70%, optionally at least about 80%, preferably at least about 85%, more preferably at least about 90% and most preferably at least about 95% homologous to the sequence AHTE (SEQ ID NO:499) in HUMHPA1B_PEA_{—}1_P106 (SEQ ID NO:143).

According to preferred embodiments of the present invention, there is provided an isolated chimeric polypeptide encoding for HUMHPA1B_PEA_{—}1_P107 (SEQ ID NO:144), comprising a first amino acid sequence being at least 90% homologous to MSALGAVIALLLWGQLFAVDSGNDVTDI corresponding to amino acids 1-28 of HPT_HUMAN (SEQ ID NO:131), which also corresponds to amino acids 1-28 of HUMHPA1B_PEA_{—}1_P107 (SEQ ID NO:144), a second amino acid sequence being at least 90% homologous to ADDGCPKPPEIAHGYVEHSVRYQCKNYYKLRTEGDGVYTLNNEKQWINKAVGDKLPE CEAVCGKPKNPANPVQRILGGHLDAKGSFPWQAKMVSHHNLTT corresponding to amino acids 88-187 of HPT_HUMAN (SEQ ID NO:131), which also corresponds to amino acids 29-128 of HUMHPA1B_PEA_{—}1_P107 (SEQ ID NO:144), and a third amino acid sequence being at least 70%, optionally at least 80%, preferably at least 85%, more preferably at least 90% and most preferably at least 95% homologous to a polypeptide having the sequence VPLPFTTWRRTPGMRLGS (SEQ ID NO:500) corresponding to amino acids 129-146 of HUMHPA1B_PEA_{—}1_P107 (SEQ ID NO:144), wherein said first amino acid sequence, second amino acid sequence and third amino acid sequence are contiguous and in a sequential order.

According to preferred embodiments of the present invention, there is provided an isolated chimeric polypeptide encoding for an edge portion of HUMHPA1B_PEA_{—}1_P107 (SEQ ID NO:144), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IA, having a structure as follows: a sequence starting from any of amino acid numbers 28-x to 28; and ending at any of amino acid numbers 29+((n−2)−x), in which x varies from 0 to n-2.

According to preferred embodiments of the present invention, there is provided an isolated polypeptide encoding for a tail of HUMHPA1B_PEA_{—}1_P107 (SEQ ID NO:144), comprising a polypeptide being at least 70%, optionally at least about 80%, preferably at least about 85%, more preferably at least about 90% and most preferably at least about 95% homologous to the sequence VPLPFTTWRRTPGMRLGS (SEQ ID NO:500) in HUMHPA1B_PEA_{—}1_P107 (SEQ ID NO:144).

According to preferred embodiments of the present invention, there is provided an isolated chimeric polypeptide encoding for HUMHPA1B_PEA_{—}1_P115 (SEQ ID NO:145), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEA corresponding to amino acids 1-88 of HPT_HUMAN (SEQ ID NO:131), which also corresponds to amino acids 1-88 of HUMHPA1B_PEA_{—}1_P115 (SEQ ID NO:145), and a second amino acid sequence being at least 70%, optionally at least 80%, preferably at least 85%, more preferably at least 90% and most preferably at least 95% homologous to a polypeptide having the sequence GGC corresponding to amino acids 89-91 of HUMHPA1B_PEA_{—}1_P115 (SEQ ID NO:145), wherein said first amino acid sequence and second amino acid sequence are contiguous and in a sequential order.

According to preferred embodiments of the present invention, there is provided an isolated chimeric polypeptide encoding for HUMELAM1A_P2 (SEQ ID NO:31), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETINNYTCKCDPGFSGLKC EQIVNCTALESPEHGSLVCSHPLGNFSYNSSCSISCDRGYLPSSMETMQCMSSGEWSAPI PACNVVECDAVTNPANGFVECFQNPGSFPWNTTCTFDCEEGFELMGAQSLQCTSSGNW DNEKPTCKAVTCRAVRQPQNGSVRCSHSPAGEFTFKSSCNFTCEEGFMLQGPAQVECT TQGQWTQQIPVCEAFQCTALSNPERGYMNCLPSASGSFRYGSSCEFSCEQGFVLKGSKR LQCGPTGEWDNEKPTCE corresponding to amino acids 1-426 of LEM2_HUMAN (SEQ ID NO:30), which also corresponds to amino acids 1-426 of HUMELAM1A_P2 (SEQ ID NO:31), and a second amino acid sequence being at least 70%, optionally at least 80%, preferably at least 85%, more preferably at least 90% and most preferably at least 95% homologous to a polypeptide having the sequence GTVFVFILF (SEQ ID NO:501) corresponding to amino acids 427-435 of HUMELAM1A_P2 (SEQ ID NO:31, wherein said first amino acid sequence and second amino acid sequence are contiguous and in a sequential order.

According to preferred embodiments of the present invention, there is provided an isolated polypeptide encoding for a tail of HUMELAM1A_P2 (SEQ ID NO:31, comprising a polypeptide being at least 70%, optionally at least about 80%, preferably at least about 85%, more preferably at least about 90% and most preferably at least about 95% homologous to the sequence GTVFVFILF (SEQ ID NO:501) in HUMELAM1A_P2 (SEQ ID NO:31).

According to preferred embodiments of the present invention, there is provided an isolated chimeric polypeptide encoding for S71513_P2 (SEQ ID NO:9), comprising a first amino acid sequence being at least 90% homologous to MKVSAALLCLLLIAATFIPQGLAQPDAINAPVTCCYNFTNRKISVQRLASYRRITSSKCP KEAV corresponding to amino acids 1-64 of SY02_HUMAN, which also corresponds to amino acids 1-64 of S71513_P2 (SEQ ID NO:9), and a second amino acid sequence being at least 70%, optionally at least 80%, preferably at least 85%, more preferably at least 90% and most preferably at least 95% homologous to a polypeptide having the sequence M corresponding to amino acids 65-65 of S71513_P2 (SEQ ID NO:9, wherein said first amino acid sequence and second amino acid sequence are contiguous and in a sequential order.

According to preferred embodiments of the present invention, there is provided an isolated chimeric polypeptide encoding for HUMELAM1A_P2 (SEQ ID NO:32), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETINNYTCKCDPGFSGLKC EQIVNCTALESPEHGSLVCSHPLGNFSYNSSCSISCDRGYLPSSMETMQCMSSGEWSAPI PACN corresponding to amino acids 1-238 of LEM2_HUMAN (SEQ ID NO:30), which also corresponds to amino acids 1-238 of HUMELAM1A_P2 (SEQ ID NO:32, and a second amino acid sequence being at least 70%, optionally at least 80%, preferably at least 85%, more preferably at least 90% and most preferably at least 95% homologous to a polypeptide having the sequence GKSL (SEQ ID NO:502) corresponding to amino acids 239-242 of HUMELAM1A_P2 (SEQ ID NO:32), wherein said first amino acid sequence and second amino acid sequence are contiguous and in a sequential order.

According to preferred embodiments of the present invention, there is provided an isolated polypeptide encoding for a tail of HUMELAM1A_P2 (SEQ ID NO:32, comprising a polypeptide being at least 70%, optionally at least about 80%, preferably at least about 85%, more preferably at least about 90% and most preferably at least about 95% homologous to the sequence GKSL (SEQ ID NO:502) in HUMELAM1A_P2 (SEQ ID NO:32).

According to preferred embodiments of the present invention, there is provided an isolated chimeric polypeptide encoding for HUMELAM1A_P2 (SEQ ID NO:33), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETINNYTCKCDPGFSGLKC EQ corresponding to amino acids 1-176 of LEM2_HUMAN (SEQ ID NO:30, which also corresponds to amino acids 1-176 of HUMELAM1A_P2 (SEQ ID NO:33), and a second amino acid sequence being at least 70%, optionally at least 80%, preferably at least 85%, more preferably at least 90% and most preferably at least 95% homologous to a polypeptide having the sequence SKSGSCLFLHLRW (SEQ ID NO:503) corresponding to amino acids 177-189 of HUMELAM1A_P2 (SEQ ID NO:33), wherein said first amino acid sequence and second amino acid sequence are contiguous and in a sequential order.

According to preferred embodiments of the present invention, there is provided an isolated polypeptide encoding for a tail of HUMELAM1A_P2 (SEQ ID NO:33), comprising a polypeptide being at least 70%, optionally at least about 80%, preferably at least about 85%, more preferably at least about 90% and most preferably at least about 95% homologous to the sequence SKSGSCLFLHLRW (SEQ ID NO:503) in HUMELAM1A_P2 (SEQ ID NO:33).

According to preferred embodiments of the present invention, there is provided an antibody capable of specifically binding to an epitope of an amino acid sequence as described herein. Optionally and preferably, the amino acid sequence corresponds to a bridge, edge portion, tail, head or insertion as in any of the above described embodiments. For example, the amino acid sequence may optionally correspond to a bridge including amino acids 64 and 65 of SEQ ID NO: 9, of at least about 10 amino acids (amino acids 55-65 of SEQ ID NO:9), preferably at least about 20 amino acids (amino acids 45-65 of SEQ ID NO:9), more preferably at least about 30 amino acids (amino acids 35-65 of SEQ ID NO:9) and most preferably at least about 40 amino acids (amino acids 25-65 of SEQ ID NO:9) in length. More preferably, the antibody is capable of differentiating between a splice variant having the epitope and a corresponding known protein.

According to preferred embodiments of the present invention, there is provided kit for detecting endometriosis, comprising a kit detecting overexpression of a splice variant according to the above described embodiments. Optionally, the kit comprises a NAT-based technology. Also optionally, the kit further comprises at least one primer pair capable of selectively hybridizing to a nucleic acid sequence according to any of the above described embodiments. Preferably, the kit further comprises at least one oligonucleotide capable of selectively hybridizing to a nucleic acid sequence according to any of the above described embodiments. More preferably, the kit comprises an antibody as described herein. Most preferably, the kit further comprises at least one reagent for performing an ELISA or a Western blot.

According to preferred embodiments of the present invention, there is provided a method for detecting endometriosis, comprising detecting overexpression and/or underexpression of a splice variant according to any of the above described embodiments. Optionally, detecting overexpression is performed with a NAT-based technology. Alternatively, detecting overexpression is performed with an immunoassay. Preferably, the immunoassay comprises an antibody according to any of the above described embodiments.

According to preferred embodiments of the present invention, there is provided a biomarker capable of detecting endometriosis, comprising any of the above nucleic acid sequences or a fragment thereof, or any of the above amino acid sequences or a fragment thereof.

According to preferred embodiments of the present invention, there is provided method for screening for endometriosis, comprising detecting endometriosis cells with a biomarker or an antibody or a method or assay according to any of the above described embodiments or as described herein.

According to preferred embodiments of the present invention, there is provided a method for diagnosing endometriosis, comprising detecting endometriosis cells with a biomarker or an antibody or a method or assay according to any of the above described embodiments or as described herein.

According to preferred embodiments of the present invention, there is provided a method for monitoring disease progression and/or treatment efficacy and/or relapse of endometriosis, comprising detecting endometriosis cells with a biomarker or an antibody or a method or assay according to any of the above described embodiments or as described herein.

According to preferred embodiments of the present invention, there is provided a method of selecting a therapy for endometriosis, comprising detecting endometriosis cells with a biomarker or an antibody or a method or assay according to any of the above described embodiments or as described herein, and selecting a therapy according to the detection.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a comparison of the human and mouse CHL2 variant I and CHL proteins.

FIG. 2 shows a schematic representation of the human and mouse CHL2 and CHL genes (sequence identification numbers as for FIG. 1).

FIG. 3 shows alternative splicing of the hCHL2 gene.

### DESCRIPTION OF PREFERRED EMBODIMENTS

The present invention is of novel markers for endometriosis that are both sensitive and accurate.

These markers are differentially expressed, and preferably in endometriosis specifically, as opposed to normal tissues. The measurement of these markers, alone or in combination, in patient samples provides information that the diagnostician can correlate with a probable diagnosis of endometriosis. The markers of the present invention, alone or in combination, show a high degree of differential detection between normal and endometriosis states. The markers of the present invention, alone or in combination, can be used for prognosis, prediction, screening, early diagnosis, staging, therapy selection and treatment monitoring of endometriosis. For example, optionally and preferably, these markers may be used for staging endometriosis and/or monitoring the progression of the disease. Also, one or more of the markers may optionally be used in combination with one or more other endometriosis markers (other than those described herein).

Biomolecular sequences (amino acid and/or nucleic acid sequences) uncovered using the methodology of the present invention and described herein can be efficiently utilized as tissue or pathological markers and/or as drugs or drug targets for treating or preventing a disease.

These markers are specifically released to the bloodstream under conditions of endometriosis, and/or are otherwise expressed at a much higher level and/or specifically expressed in endometrial tissue or cells. The measurement of these markers, alone or in combination, in patient samples provides information that the diagnostician can correlate with a probable diagnosis of endometriosis.

The present invention therefore also relates to diagnostic assays for endometriosis, and methods of use of such markers for detection of endometriosis, optionally and preferably in a sample taken from a subject (patient), which is more preferably some type of blood sample.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between a unique insertion and a “known protein” portion of a variant. Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13.37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49−x (for example) is not less than 1, nor 50+((n−2)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to the present invention, the splice variants described herein are non-limiting examples of markers for diagnosing endometriosis. Each splice variant marker of the present invention can be used alone or in combination, for various uses, including but not limited to, prognosis, prediction, screening, early diagnosis, determination of progression, therapy selection and treatment monitoring of endometriosis.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to other preferred embodiments of the present invention, a splice variant protein or a fragment thereof, or a splice variant nucleic acid sequence or a fragment thereof, may be featured as a biomarker for detecting endometriosis, such that a biomarker may optionally comprise any of the above.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Baneiji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) Hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) Hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) Hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. NatI. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Finally, the present invention also encompasses fragments of the above described polypeptides and polypeptides having mutations, such as deletions, insertions or substitutions of one or more amino acids, either naturally occurring or artificially induced, either randomly or in a targeted fashion. Similarly, homology (identity) for nucleic acid sequences is given herein as determined by BlastN software of the National Center of Biotechnology Information (NCBI) using default parameters, which preferably include using the DUST filter program, and also preferably include having an E value of 10, filtering low complexity sequences and a word size of 11.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Table 1 Non-Conventional or Modified Amino Acids which can be Used with the Present Invention.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3- | Nbhe |
| | | diphenylpropyl)glycine | |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo | Mhphe |
| | | phenylalanine | |
| L-α-methylisoleucine | Mile | N-(2-methylthioethyl)glycine | Nmet |
| D-N-methylglutamine | Dnmgln | N-(3- | Narg |
| | | guanidinopropyl)glycine | |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N- | Nmchexa | D-N-methylmethionine | Dnmmet |
| methylcyclohexylalanine | | | |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α- | Mhphe |
| | | methylhomophenylalanine | |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) Such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) Such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47): 15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

The following sections relate to Candidate Marker Examples. It should be noted that Table numbering is restarted within each example relating to each cluster (each such section begins with “Description for Cluster” followed by the name of the cluster).

Candidate Marker Examples Section

This Section relates to Examples of sequences and markers according to the present invention.

Description of the methodology undertaken to uncover the biomolecular sequences of the present invention

Human ESTs and cDNAs were obtained from GenBank versions 136 (Jun. 15, 2003 ftp.ncbi.nih.gov/genbank/release.notes/bgb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) Section and the human mRNA sequences from the primate (GBPR1) Section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as U.S. 20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

### Description for Cluster S71513

Cluster S71513 features 1 transcript(s) and 6 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| S71513_T2 | 1 |

TABLE 2
Segments of interest

| Segment Name | Sequence ID No. |
|---|---|
| S71513_node_0 | 2 |
| S71513_node_5 | 3 |
| S71513_node_6 | 4 |
| S71513_node_8 | 5 |
| S71513_node_1 | 6 |
| S71513_node_4 | 7 |

TABLE 3
Proteins of interest

| Protein Name | Sequence ID No. | Corresponding Transcript(s) |
|---|---|---|
| S71513_P2 | 9 | S71513_T2 (SEQ ID NO: 1) |

These sequences are variants of the known protein Small inducible cytokine A2 precursor (SEQ ID NO:8) (SwissProt accession identifier SY02_HUMAN; known also according to the synonyms CCL2; Monocyte chemotactic protein 1; MCP-1; Monocyte chemoattractant protein-1; Monocyte chemotactic and activating factor; MCAF; Monocyte secretory protein JE; HCl 1), referred to herein as the previously known protein.

Protein Small inducible cytokine A2 precursor (SEQ ID NO:8) is known or believed to have the following function(s): chemotactic factor that attracts monocytes and basophils but not neutrophils or eosinophils. Augments monocyte anti-tumor activity. Has been implicated in the pathogenesis of diseases characterized by monocytic infiltrates, like psoriasis, rheumatoid arthritis or atherosclerosis. May be involved in the recruitment of monocytes into the arterial wall during the disease process of atherosclerosis. Binds to CCR2 and CCR4. The sequence for protein Small inducible cytokine A2 precursor (SEQ ID NO:8) is given at the end of the application, as “Small inducible cytokine A2 precursor amino acid sequence” (SEQ ID NO:8). Known polymorphisms for this sequence are as shown in Table 4.

TABLE 4
Amino acid mutations for Known Protein

| SNP position(s) on | |
|---|---|
| amino acid sequence | Comment |
| 76 | A -> T./FTId = VAR_001632. |
| 24 | Missing: Loss of activity. |
| 25-32 | Missing: Loss of activity. |
| 24-85 | MISSING: 90% REDUCTION IN ACTIVITY. |
| 24-91 | MISSING: 83% REDUCTION IN ACTIVITY. |
| 26 | D->A: 90% REDUCTION IN ACTIVITY. |
| 29 | N->A: 50% REDUCTION IN ACTIVITY. |
| 47 | R->F: 95% REDUCTION IN ACTIVITY. |
| 50 | S->Q: 40% REDUCTION IN ACTIVITY. |
| 51 | Y->D: LOSS OF ACTIVITY. |
| 53 | R->L: LOSS OF ACTIVITY. |
| 91 | D->L: 90% REDUCTION IN ACTIVITY. |

Protein Small inducible cytokine A2 precursor (SEQ ID NO:8) localization is believed to be Secreted.

Rong et al reported that MCP-1 causes (or at least is associated with) an inflammatory action of peritoneal fluid of women with endometriosis (Fertil Steril. 2002 October; 78(4):843-8). Therefore, variants according to the present invention are believed to be useful as diagnostic markers for endometriosis.

The following GO Annotation(s) apply to the previously known protein. The following annotation(s) were found: protein amino acid phosphorylation; calcium ion homeostasis; anti-apoptosis; chemotaxis; inflammatory response; humoral defense mechanism; cell adhesion; G-protein signaling, coupled to cyclic nucleotide second messenger; JAK-STAT cascade; cell-cell signaling; response to pathogenic bacteria; viral genome replication, which are annotation(s) related to Biological Process; protein kinase; ligand; chemokine, which are annotation(s) related to Molecular Function; and extracellular space; membrane, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster S71513 features 1 transcript(s), which were listed in Table 1 above. These transcript(s) encode for protein(s) which are variant(s) of protein Small inducible cytokine A2 precursor (SEQ ID NO:8). A description of each variant protein according to the present invention is now provided.

Variant protein S71513_P2 (SEQ ID NO:9) according to the present invention has an amino acid sequence as given at the end of the application; it is encoded by transcript(s) S71513_T2 (SEQ ID NO:1). An alignment is given to the known protein (Small inducible cytokine A2 precursor (SEQ ID NO:8)) at the end of the application. One or more alignments to one or more previously published protein sequences are given at the end of the application. A brief description of the relationship of the variant protein according to the present invention to each such aligned protein is as follows:

Comparison report between S71513_P2 (SEQ ID NO:9) and SY02_HUMAN (SEQ ID NO:8):

1. An isolated chimeric polypeptide encoding for S71513_P2 (SEQ ID NO:9), comprising a first amino acid sequence being at least 90% homologous to MKVSAALLCLLLIAATFIPQGLAQPDAINAPVTCCYNFTNRKISVQRLASYRRITSSKCP KEAV corresponding to amino acids 1-64 of SY02_HUMAN (SEQ ID NO:8), which also corresponds to amino acids 1-64 of S71513_P2 (SEQ ID NO:9), and a second amino acid sequence being at least 70%, optionally at least 80%, preferably at least 85%, more preferably at least 90% and most preferably at least 95% homologous to a polypeptide having the sequence M corresponding to amino acids 65-65 of S71513_P2 (SEQ ID NO:9, wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S71513_P2 (SEQ ID NO:9) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein S71513_P2 (SEQ ID NO:9) Sequence provides support for the deduced sequence of this variant protein according to the present invention).

TABLE 5
Amino acid mutations

| SNP position(s) on amino acid | Alternative | |
|---|---|---|
| sequence | amino acid(s) | Previously known SNP? |
| 15 | A -> | No |
| 15 | A -> G | No |
| 22 | L -> P | No |

The glycosylation sites of variant protein S71513_P2 (SEQ ID NO:9), as compared to the known protein Small inducible cytokine A2 precursor (SEQ ID NO:8), are described in Table 6 (given according to their position(s) on the amino acid sequence in the first column; the second column indicates whether the glycosylation site is present in the variant protein; and the last column indicates whether the position is different on the variant protein).

TABLE 6
Glycosylation site(s)

| Position(s) on known amino | Present in | |
|---|---|---|
| acid sequence | variant protein? | Position in variant protein? |
| 37 | yes | 37 |

The phosphorylation sites of variant protein S71513_P2 (SEQ ID NO:9), as compared to the known protein Small inducible cytokine A2 precursor (SEQ ID NO:8), are described in Table 7 (given according to their position(s) on the amino acid sequence in the first column; the second column indicates whether the phosphorylation site is present in the variant protein; and the last column indicates whether the position is different on the variant protein).

TABLE 7
Phosphorylation site(s)

| Position(s) on known amino | | Position in |
|---|---|---|
| acid sequence | Present in variant protein? | variant protein? |
| 24 | yes | 24 |

Variant protein S71513_P2 (SEQ ID NO:9) is encoded by the following transcript(s): S71513_T2 (SEQ ID NO:1), for which the sequence(s) is/are given at the end of the application. The coding portion of transcript S71513_T2 (SEQ ID NO:1) is shown in bold; this coding portion starts at position 341 and ends at position 535. The transcript also has the following SNPs as listed in Table 8 (given according to their position on the nucleotide sequence, with the alternative nucleic acid listed; the last column indicates whether the SNP is known or not; the presence of known SNPs in variant protein S71513_P2 (SEQ ID NO:9) Sequence provides support for the deduced sequence of this variant protein according to the present invention).

TABLE 8
Nucleic acid SNPs

| SNP position on nucleotide | | Previously |
|---|---|---|
| sequence | Alternative nucleic acid | known SNP? |
| 219 | G -> T | Yes |
| 222 | C -> T | Yes |
| 383 | G -> | No |
| 384 | C -> | No |
| 384 | C -> G | No |
| 403 | G -> T | No |
| 405 | T -> C | No |
| 439 | C -> T | No |
| 445 | T -> C | Yes |
| 559 | C -> T | Yes |
| 963 | A -> G | No |
| 1045 | A -> G | No |
| 1045 | A -> T | No |
| 1087 | C -> T | Yes |
| 1090 | T -> | No |
| 1090 | T -> G | No |
| 1110 | T -> | No |
| 1127 | A -> | No |
| 1203 | T -> | No |
| 1203 | T -> G | No |
| 1247 | C -> T | Yes |
| 1360 | -> G | No |
| 1360 | -> T | No |
| 1388 | T -> | No |

As noted above, cluster S71513 features 6 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster S71513_node_{—}0 (SEQ ID NO:2) according to the present invention is supported by 292 libraries. The number of libraries was determined as previously described. This segment can be found in the following transcript(s): S71513_T2 (SEQ ID NO:1). Table 9 below describes the starting and ending position of this segment on each transcript.

TABLE 9
Segment location on transcripts

| | Segment | Segment |
|---|---|---|
| Transcript name | starting position | ending position |
| S71513_T2 (SEQ ID NO: 1) | 1 | 387 |

Segment cluster S71513_node_(SEQ ID NO:3) according to the present invention is supported by 39 libraries. The number of libraries was determined as previously described. This segment can be found in the following transcript(s): S71513-T2 (SEQ ID NO:1). Table 10 below describes the starting and ending position of this segment on each transcript.

TABLE 10
Segment location on transcripts

| | Segment | Segment |
|---|---|---|
| Transcript name | starting position | ending position |
| S71513_T2 (SEQ ID NO: 1) | 535 | 916 |

Segment cluster S71513_node_{—}6 (SEQ ID NO:4) according to the present invention is supported by 326 libraries. The number of libraries was determined as previously described. This segment can be found in the following transcript(s): S71513-T2 (SEQ ID NO:1). Table 11 below describes the starting and ending position of this segment on each transcript.

TABLE 11
Segment location on transcripts

| | Segment | Segment |
|---|---|---|
| Transcript name | starting position | ending position |
| S71513_T2 (SEQ ID NO: 1) | 917 | 1272 |

Segment cluster S71513_node_{—}8 (SEQ ID NO:5) according to the present invention is supported by 165 libraries. The number of libraries was determined as previously described. This segment can be found in the following transcript(s): S71513_T2 (SEQ ID NO:1). Table 12 below describes the starting and ending position of this segment on each transcript.

TABLE 12
Segment location on transcripts

| | Segment | Segment |
|---|---|---|
| Transcript name | starting position | ending position |
| S71513_T2 (SEQ ID NO: 1) | 1273 | 1404 |

According to an optional embodiment of the present invention, short segments related to the above cluster are also provided. These segments are up to about 120 bp in length, and so are included in a separate description.

Segment cluster S71513_node_{—}1 (SEQ ID NO:6) according to the present invention is supported by 296 libraries. The number of libraries was determined as previously described. This segment can be found in the following transcript(s): S71513_T2 (SEQ ID NO:1). Table 13 below describes the starting and ending position of this segment on each transcript.

TABLE 13
Segment location on transcripts

| | Segment | Segment |
|---|---|---|
| Transcript name | starting position | ending position |
| S71513_T2 (SEQ ID NO: 1) | 388 | 416 |

Segment cluster S71513_node_{—}4 (SEQ ID NO:7) according to the present invention is supported by 319 libraries. The number of libraries was determined as previously described. This segment can be found in the following transcript(s): S71513 T2 (SEQ ID NO:1). Table 14 below describes the starting and ending position of this segment on each transcript.

TABLE 14
Segment location on transcripts

| | Segment | Segment |
|---|---|---|
| Transcript name | starting position | ending position |
| S71513_T2 (SEQ ID NO: 1) | 417 | 534 |

Variant protein alignment to the previously known protein:

| Sequence name: SY02_HUMAN (SEQ ID NO: 8) | | | |
|---|---|---|---|
| Sequence documentation: | | | |
| Alignment of: S71513_P2 (SEQ ID NO: 9) × SY02_HUMAN (SEQ ID NO: 8) .. | | | |
| Alignment segment 1/1: | | | |
| Quality: | 619.00 | Escore: | 0 |
| Matching length: | 65 | Total length: | 65 |
| Matching Percent Similarity: | 100.00 | Matching Percent Identity: | 98.46 |
| Total Percent Similarity: | 100.00 | Total Percent Identity: | 98.46 |
| Gaps: | 0 | | |

| Alignment: | | |
|---|---|---|
| | . . . . . | |
| 1 | MKVSAALLCLLLIAATFIPQGLAQPDAINAPVTCCYNFTNRKISVQRLAS | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MKVSAALLCLLLIAATFIPQGLAQPDAINAPVTCCYNFTNRKISVQRLAS | 50 |
| | . | |
| 51 | YRRITSSKCPKEAVM | 65 |
| | \|\|\|\|\|\|\|\|\|\|\|\|\|\|: | |
| 51 | YRRITSSKCPKEAVI | 65 |

### Description for Cluster HUMELAM1A

Cluster HUMELAM1A features 3 transcript(s) and 17 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID No. |
|---|---|
| HUMELAM1A_T1 | 10 |
| HUMELAM1A_T5 | 11 |
| HUMELAM1A_T6 | 12 |

TABLE 2
Segments of interest

| Segment Name | SEQ ID No. |
|---|---|
| HUMELAM1A_node_5 | 13 |
| HUMELAM1A_node_8 | 14 |
| HUMELAM1A_node_10 | 15 |
| HUMELAM1A_node_11 | 16 |
| HUMELAM1A_node_13 | 17 |
| HUMELAM1A_node_15 | 18 |
| HUMELAM1A_node_18 | 19 |
| HUMELAM1A_node_19 | 20 |
| HUMELAM1A_node_20 | 21 |
| HUMELAM1A_node_22 | 22 |
| HUMELAM1A_node_33 | 23 |
| HUMELAM1A_node_0 | 24 |
| HUMELAM1A_node_2 | 25 |
| HUMELAM1A_node_7 | 26 |
| HUMELAM1A_node_24 | 27 |
| HUMELAM1A_node_26 | 28 |
| HUMELAM1A_node_29 | 29 |

TABLE 3
Proteins of interest

| Protein Name | SEQ ID No. | Corresponding Transcript(s) |
|---|---|---|
| HUMELAM1A_P2 | 31 | HUMELAM1A_T1 (SEQ ID |
| | | NO: 10) |
| HUMELAM1A_P4 | 32 | HUMELAM1A_T5 (SEQ ID |
| | | NO: 11) |
| HUMELAM1A_P5 | 33 | HUMELAM1A_T6 (SEQ ID |
| | | NO: 12) |

These sequences are variants of the known protein E-selectin precursor (SEQ ID NO:30) (SwissProt accession identifier LEM2_HUMAN (SEQ ID NO:30; known also according to the synonyms Endothelial leukocyte adhesion molecule 1; ELAM-1; Leukocyte-endothelial cell adhesion molecule 2; LECAM2; CD62E antigen), referred to herein as the previously known protein.

Protein E-selectin precursor (SEQ ID NO:30) is known or believed to have the following function(s): expressed on cytokine induced endothelial cells and mediates their binding to leukocytes. The ligand recognized by ELAM-1 is sialyl-lewis X (alpha(1->3)fucosylated derivatives of polylactosamine that are found at the nonreducing termini of glycolipids). The sequence for protein E-selectin precursor is given at the end of the application, as “E-selectin precursor amino acid sequence” (SEQ ID NO:30). Known polymorphisms for this sequence are as shown in Table 4.

TABLE 4
Amino acid mutations for Known Protein

| SNP position(s) on | |
|---|---|
| amino acid sequence | Comment |
| 21 | A -> S. /FTId = VAR_014300. |
| 31 | M -> I. /FTId = VAR_014301. |
| 130 | C -> W (in dbSNP: 5360). /FTId = VAR_011790. |
| 149 | S -> R (polymorphism associated with coronary |
| | artery disease; dbSNP: 5361). /FTId = |
| | VAR_004191. |
| 257 | Q -> P. /FTId = VAR_014302. |
| 295 | E -> K (in dbSNP: 5364). /FTId = VAR_011791. |
| 421 | E -> Q (in dbSNP: 5366). /FTId = VAR_011792. |
| 468 | H -> Y (in dbSNP: 5368). /FTId = VAR_011793. |
| 550 | P -> S. /FTId = VAR_014303. |
| 575 | L -> F (in dbSNP: 5355). /FTId = VAR_011794. |

Protein E-selectin precursor (SEQ ID NO:30) localization is believed to be Type I membrane protein.

Yang et al reported that E-selectin may be involved in, or related to, endometrisosis (Best Pract Res Clin Obstet Gynaecol. 2004 April; 18(2):305-18). Therefore, variants according to the present invention are believed to be useful as diagnostic markers for endometriosis.

The previously known protein also has the following indication(s) and/or potential therapeutic use(s): Ischaemia, cerebral. It has been investigated for clinicat/therapeutic use in humans, for example as a target for an antibody or small molecule, and/or as a direct therapeutic; available information related to these investigations is as follows. Potential pharmaceutically related or therapeutically related activity or activities of the previously known protein are as follows: E selectin agonist; Immunostimulant. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anti-inflammatory; Neuroprotective.

The following GO Annotation(s) apply to the previously known protein. The following annotation(s) were found: inflammatory response; cell adhesion; heterophilic cell adhesion, which are annotation(s) related to Biological Process; protein binding; sugar binding, which are annotation(s) related to Molecular Function; and plasma membrane; integral membrane protein, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster HUMELAM1A features 3 transcript(s), which were listed in Table 1 above. These transcript(s) encode for protein(s) which are variant(s) of protein E-selectin precursor (SEQ ID NO:30). A description of each variant protein according to the present invention is now provided.

Variant protein HUMELAM1A_P2 (SEQ ID NO:31) according to the present invention has an amino acid sequence as given at the end of the application; it is encoded by transcript(s) HUMELAM1A_T1 (SEQ ID NO:10). An alignment is given to the known protein (E-selectin precursor (SEQ ID NO:30) at the end of the application. One or more alignments to one or more previously published protein sequences are given at the end of the application. A brief description of the relationship of the variant protein according to the present invention to each such aligned protein is as follows:

Comparison report between HUMELAM1A_P2 (SEQ ID NO:31) and LEM2_HUMAN (SEQ ID NO:30):

1. An isolated chimeric polypeptide encoding for HUMELAM1A_P2 (SEQ ID NO:31), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETNNYTCKCDPGFSGLKC EQIVNCTALESPEHGSLVCSHPLGNFSYNSSCSISCDRGYLPSSMETMQCMSSGEWSAPI PACNVVECDAVTNPANGFVECFQNPGSFPWNTTCTFDCEEGFELMGAQSLQCTSSGNW DNEKPTCKAVTCRAVRQPQNGSVRCSHSPAGEFTFKSSCNFTCEEGFMLQGPAQVECT TQGQWTQQIPVCEAFQCTALSNPERGYMNCLPSASGSFRYGSSCEFSCEQGFVLKGSKR LQCGPTGEWDNEKPTCE corresponding to amino acids 1-426 of LEM2_HUMAN (SEQ ID NO:30), which also corresponds to amino acids 1-426 of HUMELAM1A_P2 (SEQ ID NO:31), and a second amino acid sequence being at least 70%, optionally at least 80%, preferably at least 85%, more preferably at least 90% and most preferably at least 95% homologous to a polypeptide having the sequence GTVFVFILF (SEQ ID NO:501) corresponding to amino acids 427-435 of HUMELAM1A_P2 (SEQ ID NO:31), wherein said first amino acid sequence and second amino acid sequence are contiguous and in a sequential order.

2. An isolated polypeptide encoding for a tail of HUMELAM1A_P2 (SEQ ID NO:31), comprising a polypeptide being at least 70%, optionally at least about 80%, preferably at least about 85%, more preferably at least about 90% and most preferably at least about 95% homologous to the sequence GTVFVFILF (SEQ ID NO:501) in HUMELAM1A_P2 (SEQ ID NO:31).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMELAM1A_P2 (SEQ ID NO:31) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein HUMELAM1A_P2 (SEQ ID NO:31) Sequence provides support for the deduced sequence of this variant protein according to the present invention).

TABLE 5
Amino acid mutations

| SNP position(s) on | | |
|---|---|---|
| amino acid | | |
| sequence | Alternative amino acid(s) | Previously known SNP? |
| 21 | A -> S | Yes |
| 31 | M -> I | Yes |
| 130 | C -> W | Yes |
| 149 | S -> R | Yes |
| 257 | Q -> P | Yes |
| 295 | E -> K | Yes |
| 421 | E -> Q | Yes |

The glycosylation sites of variant protein HUMELAM1A_P2 (SEQ ID NO:31), as compared to the known protein E-selectin precursor (SEQ ID NO:30), are described in Table 6 (given according to their position(s) on the amino acid sequence in the first column; the second column indicates whether the glycosylation site is present in the variant protein; and the last column indicates whether the position is different on the variant protein).

TABLE 6
Glycosylation site(s)

| Position(s) on known amino | Present | Position in variant |
|---|---|---|
| acid sequence | in variant protein? | protein? |
| 199 | yes | 199 |
| 203 | yes | 203 |
| 312 | yes | 312 |
| 145 | yes | 145 |
| 332 | yes | 332 |
| 503 | no | |
| 265 | yes | 265 |
| 160 | yes | 160 |
| 25 | yes | 25 |
| 527 | no | |
| 179 | yes | 179 |

Variant protein HUMELAM1A_P2 (SEQ ID NO:31) is encoded by the following transcript(s): HUMELAM1A_T1 (SEQ ID NO:10), for which the sequence(s) is/are given at the end of the application. The coding portion of transcript HUMELAM1A_T1 (SEQ ID NO:10) is shown in bold; this coding portion starts at position 164 and ends at position 1468. The transcript also has the following SNPs as listed in Table 7 (given according to their position on the nucleotide sequence, with the alternative nucleic acid listed; the last column indicates whether the SNP is known or not; the presence of known SNPs in variant protein HUMELAM1A_P2 (SEQ ID NO:31) Sequence provides support for the deduced sequence of this variant protein according to the present invention).

TABLE 7
Nucleic acid SNPs

| SNP position on nucleotide | | Previously |
|---|---|---|
| sequence | Alternative nucleic acid | known SNP? |
| 43 | A -> G | Yes |
| 65 | A -> G | Yes |
| 145 | G -> T | Yes |
| 224 | G -> T | Yes |
| 256 | G -> T | Yes |
| 436 | A -> G | Yes |
| 439 | A -> G | Yes |
| 553 | C -> G | Yes |
| 608 | A -> C | Yes |
| 904 | T -> C | Yes |
| 933 | A -> C | Yes |
| 1036 | T -> C | Yes |
| 1046 | G -> A | Yes |
| 1423 | C -> T | Yes |
| 1424 | G -> C | Yes |
| 1475 | A -> G | Yes |
| 1524 | T -> A | Yes |
| 1565 | T -> C | Yes |
| 1695 | T -> C | Yes |
| 1941 | C -> T | Yes |
| 1982 | T -> C | Yes |
| 2016 | C -> T | Yes |
| 2093 | T -> C | Yes |
| 2114 | T -> C | Yes |
| 2332 | T -> A | Yes |
| 2486 | A -> G | Yes |
| 3079 | T -> C | Yes |
| 3116 | T -> G | Yes |
| 3270 | A -> G | Yes |
| 3660 | A -> G | Yes |
| 3671 | C -> G | Yes |

Variant protein HUMELAM1A_P2 (SEQ ID NO:32) according to the present invention has an amino acid sequence as given at the end of the application; it is encoded by transcript(s) HUMELAM1A_T5 (SEQ ID NO:11. An alignment is given to the known protein (E-selectin precursor (SEQ ID NO:30)) at the end of the application. One or more alignments to one or more previously published protein sequences are given at the end of the application. A brief description of the relationship of the variant protein according to the present invention to each such aligned protein is as follows:

Comparison report between HUMELAM1A_P2 (SEQ ID NO:32) and LEM2_HUMAN (SEQ ID NO:30):

1. An isolated chimeric polypeptide encoding for HUMELAM1A_P2 (SEQ ID NO:32), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETINNYTCKCDPGFSGLKC EQIVNCTALESPEHGSLVCSHPLGNFSYNSSCSISCDRGYLPSSMETMQCMSSGEWSAPI PACN corresponding to amino acids 1-238 of LEM2_HUMAN (SEQ ID NO:30, which also corresponds to amino acids 1-238 of HUMELAM1A_P2 (SEQ ID NO:32), and a second amino acid sequence being at least 70%, optionally at least 80%, preferably at least 85%, more preferably at least 90% and most preferably at least 95% homologous to a polypeptide having the sequence GKSL (SEQ ID NO:502) corresponding to amino acids 239-242 of HUMELAM1A_P2 (SEQ ID NO:32, wherein said first amino acid sequence and second amino acid sequence are contiguous and in a sequential order.

2. An isolated polypeptide encoding for a tail of HUMELAM1A_P2 (SEQ ID NO:32), comprising a polypeptide being at least 70%, optionally at least about 80%, preferably at least about 85%, more preferably at least about 90% and most preferably at least about 95% homologous to the sequence GKSL (SEQ ID NO:502) in HUMELAM1A_P2 (SEQ ID NO:32.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMELAM1A_P2 (SEQ ID NO:32) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein HUMELAM1A_P2 (SEQ ID NO:32) Sequence provides support for the deduced sequence of this variant protein according to the present invention).

TABLE 8
Amino acid mutations

| SNP position(s) on amino acid | Alternative | |
|---|---|---|
| sequence | amino acid(s) | Previously known SNP? |
| 21 | A -> S | Yes |
| 31 | M -> I | Yes |
| 130 | C -> W | Yes |
| 149 | S -> R | Yes |

The glycosylation sites of variant protein HUMELAM1A_P2 (SEQ ID NO:32), as compared to the known protein E-selectin precursor (SEQ ID NO:30, are described in Table 9 (given according to their position(s) on the amino acid sequence in the first column; the second column indicates whether the glycosylation site is present in the variant protein; and the last column indicates whether the position is different on the variant protein).

TABLE 9
Glycosylation site(s)

| Position(s) on known amino | | Position in |
|---|---|---|
| acid sequence | Present in variant protein? | variant protein? |
| 199 | yes | 199 |
| 203 | yes | 203 |
| 312 | no | |
| 145 | yes | 145 |
| 332 | no | |
| 503 | no | |
| 265 | no | |
| 160 | yes | 160 |
| 25 | yes | 25 |
| 527 | no | |
| 179 | yes | 179 |

Variant protein HUMELAM1A_P2 (SEQ ID NO:32) is encoded by the following transcript(s): HUMELAM1A_T5 (SEQ ID NO:11), for which the sequence(s) is/are given at the end of the application. The coding portion of transcript HUMELAM1A_T5 (SEQ ID NO:11) is shown in bold; this coding portion starts at position 164 and ends at position 889. The transcript also has the following SNPs as listed in Table 10 (given according to their position on the nucleotide sequence, with the alternative nucleic acid listed; the last column indicates whether the SNP is known or not; the presence of known SNPs in variant protein HUMELAM1A_P2 (SEQ ID NO:32) Sequence provides support for the deduced sequence of this variant protein according to the present invention).

TABLE 10
Nucleic acid SNPs

| SNP position on nucleotide | | Previously |
|---|---|---|
| sequence | Alternative nucleic acid | known SNP? |
| 43 | A -> G | Yes |
| 65 | A -> G | Yes |
| 145 | G -> T | Yes |
| 224 | G -> T | Yes |
| 256 | G -> T | Yes |
| 436 | A -> G | Yes |
| 439 | A -> G | Yes |
| 553 | C -> G | Yes |
| 608 | A -> C | Yes |

Variant protein HUMELAM1A_P2 (SEQ ID NO:33) according to the present invention has an amino acid sequence as given at the end of the application; it is encoded by transcript(s) HUMELAM1A_T6 (SEQ ID NO:12). An alignment is given to the known protein (E-selectin precursor (SEQ ID NO:30) at the end of the application. One or more alignments to one or more previously published protein sequences are given at the end of the application. A brief description of the relationship of the variant protein according to the present invention to each such aligned protein is as follows:

Comparison report between HUMELAM1A_P2 (SEQ ID NO:33) and LEM2_HUMAN (SEQ ID NO:30):

1. An isolated chimeric polypeptide encoding for HUMELAM1A_P2 (SEQ ID NO:33), comprising a first amino acid sequence being at least 90% homologous to MIASQFLSALTLVLLIKESGAWSYNTSTEAMTYDEASAYCQQRYTHLVAIQNKEEIEYL NSILSYSPSYYWIGIRKVNNVWVWVGTQKPLTEEAKNWAPGEPNNRQKDEDCVEIYIK REKDVGMWNDERCSKKKLALCYTAACTNTSCSGHGECVETINbYTCKCDPGFSGLKC EQ corresponding to amino acids 1-176 of LEM2_HUMAN (SEQ ID NO:30), which also corresponds to amino acids 1-176 of HUMELAM1A_P2 (SEQ ID NO:33), and a second amino acid sequence being at least 70%, optionally at least 80%, preferably at least 85%, more preferably at least 90% and most preferably at least 95% homologous to a polypeptide having the sequence SKSGSCLFLHLRW (SEQ ID NO:503) corresponding to amino acids 177-189 of HUMELAM1A_P2 (SEQ ID NO:33), wherein said first amino acid sequence and second amino acid sequence are contiguous and in a sequential order.

2. An isolated polypeptide encoding for a tail of HUMELAM1A_P2 (SEQ ID NO:33), comprising a polypeptide being at least 70%, optionally at least about 80%, preferably at least about 85%, more preferably at least about 90% and most preferably at least about 95% homologous to the sequence SKSGSCLFLHLRW (SEQ ID NO:503) in HUMELAM1A_P2 (SEQ ID NO:33).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMELAM1A_P2 (SEQ ID NO:33) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HUMELAM1A_P2 (SEQ ID NO:33) Sequence provides support for the deduced sequence of this variant protein according to the present invention).

TABLE 11
Amino acid mutations

| SNP position(s) on amino acid | | Previously |
|---|---|---|
| sequence | Alternative amino acid(s) | known SNP? |
| 21 | A -> S | Yes |
| 31 | M -> I | Yes |
| 130 | C -> W | Yes |
| 149 | S -> R | Yes |
| 182 | C -> R | Yes |

The glycosylation sites of variant protein HUMELAM1A_P2 (SEQ ID NO:33), as compared to the known protein E-selectin precursor (SEQ ID NO:30), are described in Table 12 (given according to their position(s) on the amino acid sequence in the first column; the second column indicates whether the glycosylation site is present in the variant protein; and the last column indicates whether the position is different on the variant protein).

TABLE 12
Glycosylation site(s)

| Position(s) on known amino | | Position in |
|---|---|---|
| acid sequence | Present in variant protein? | variant protein? |
| 199 | no | |
| 203 | no | |
| 312 | no | |
| 145 | yes | 145 |
| 332 | no | |
| 503 | no | |
| 265 | no | |
| 160 | yes | 160 |
| 25 | yes | 25 |
| 527 | no | |
| 179 | no | |

Variant protein HUMELAM1A_P2 (SEQ ID NO:33) is encoded by the following ript(s): HUMELAM1A_T6 (SEQ ID NO:12), for which the sequence(s) is/are given at the end of the application. The coding portion of transcript HUMELAM1A_T6 (SEQ ID NO: 12) is shown in bold; this coding portion starts at position 164 and ends at position 730. The transcript also has the following SNPs as listed in Table 13 (given according to their position on the nucleotide sequence, with the alternative nucleic acid listed; the last column indicates whether the SNP is known or not; the presence of known SNPs in variant protein HUMELAM1A_P2 (SEQ ID NO:33) Sequence provides support for the deduced sequence of this variant protein according to the present invention).

TABLE 13
Nucleic acid SNPs

| SNP position on nucleotide | | Previously |
|---|---|---|
| sequence | Alternative nucleic acid | known SNP? |
| 43 | A -> G | Yes |
| 65 | A -> G | Yes |
| 145 | G -> T | Yes |
| 224 | G -> T | Yes |
| 256 | G -> T | Yes |
| 436 | A -> G | Yes |
| 439 | A -> G | Yes |
| 553 | C -> G | Yes |
| 608 | A -> C | Yes |
| 707 | T -> C | Yes |
| 815 | C -> T | Yes |
| 912 | T -> A | Yes |

As noted above, cluster HUMELAM1A features 17 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMELAM1A_node_{—}5 (SEQ ID NO:13) according to the present invention is supported by 16 libraries. The number of libraries was determined as previously described. This segment can be found in the following transcript(s): HUMELAM1A_T1 (SEQ ID NO:10), HUMELAM1A_T5 (SEQ ID NO:11) and HUMELAM1A_T6 (SEQ ID NO:12). Table 14 below describes the starting and ending position of this segment on each transcript.

TABLE 14
Segment location on transcripts

| | Segment | |
|---|---|---|
| | starting | Segment |
| Transcript name | position | ending position |
| HUMELAM1A_T1 (SEQ ID NO: 10) | 201 | 584 |
| HUMELAM1A_T5 (SEQ ID NO: 11) | 201 | 584 |
| HUMELAM1A_T6 (SEQ ID NO: 12) | 201 | 584 |

Segment cluster HUMELAM1A_node_{—}8 (SEQ ID NO:14) according to the present invention is supported by 1 libraries. The number of libraries was determined as previously described. This segment can be found in the following transcript(s): HUMELAM1A_T6 (SEQ ID NO:12). Table 15 below describes the starting and ending position of this segment on each transcript.

TABLE 15
Segment location on transcripts

| | Segment | |
|---|---|---|
| | starting | Segment |
| Transcript name | position | ending position |
| HUMELAM1A_T6 (SEQ ID NO: 12) | 693 | 1061 |

Segment cluster HUMELAM1A_node_{—}10 (SEQ ID NO:15) according to the present invention is supported by 15 libraries. The number of libraries was determined as previously described. This segment can be found in the following transcript(s): HUMELAM1A_T1 (SEQ ID NO:10) and HUMELAM1A_T5 (SEQ ID NO:11). Table 16 below describes the starting and ending position of this segment on each transcript.

TABLE 16
Segment location on transcripts

| | | Segment |
|---|---|---|
| | Segment | ending |
| Transcript name | starting position | position |
| HUMELAM1A_T1 (SEQ ID NO: 10) | 693 | 878 |
| HUMELAM1A_T5 (SEQ ID NO: 11) | 693 | 878 |

Segment cluster HUMELAM1A_node_{—}11 (SEQ ID NO:16) according to the present invention is supported by 3 libraries. The number of libraries was determined as previously described. This segment can be found in the following transcript(s): HUMELAM1A_T5 (SEQ ID NO:11). Table 17 below describes the starting and ending position of this segment on each transcript.

TABLE 17
Segment location on transcripts

| | | Segment |
|---|---|---|
| | Segment | ending |
| Transcript name | starting position | position |
| HUMELAM1A_T5 (SEQ ID NO: 11) | 879 | 1150 |

Segment cluster HUMELAM1A_node_{—}13 (SEQ ID NO:17) according to the present invention is supported by 10 libraries. The number of libraries was determined as previously described. This segment can be found in the following transcript(s): HUMELAM1A_T1 (SEQ ID NO:10). Table 18 below describes the starting and ending position of this segment on each transcript.

TABLE 18
Segment location on transcripts

| | | Segment |
|---|---|---|
| | Segment | ending |
| Transcript name | starting position | position |
| HUMELAM1A_T1 (SEQ ID NO: 10) | 879 | 1064 |

Segment cluster HUMELAM1A_node_{—}15 (SEQ ID NO:18) according to the present invention is supported by 10 libraries. The number of libraries was determined as previously described. This segment can be found in the following transcript(s): HUMELAM1A_T1 (SEQ ID NO:10). Table 19 below describes the starting and ending position of this segment on each transcript.

TABLE 19
Segment location on transcripts

| | | Segment |
|---|---|---|
| | Segment | ending |
| Transcript name | starting position | position |
| HUMELAM1A_T1 (SEQ ID NO: 10) | 1065 | 1253 |

Segment cluster HUMELAM1A_node_{—}18 (SEQ ID NO:19) according to the present invention is supported by 14 libraries. The number of libraries was determined as previously described. This segment can be found in the following transcript(s): HUMELAM1A_T1 (SEE ID NO:10). Table 20 below describes the starting and ending position of this segment on each transcript.

TABLE 20
Segment location on transcripts

| | | Segment |
|---|---|---|
| | Segment | ending |
| Transcript name | starting position | position |
| HUMELAM1A_T1 (SEQ ID NO: 10) | 1254 | 1442 |

Segment cluster HUMELAM1A_node_{—}19 (SEQ ID NO:20) according to the present invention is supported by 4 libraries. The number of libraries was determined as previously described. This segment can be found in the following transcript(s): HUMELAM1A_T1 (SEQ ID NO:10). Table 21 below describes the starting and ending position of this segment on each transcript.

TABLE 21
Segment location on transcripts

| | | Segment |
|---|---|---|
| | Segment | ending |
| Transcript name | starting position | position |
| HUMELAM1A_T1 (SEQ ID NO: 10) | 1443 | 1572 |

Segment cluster HUMELAM1A_node_{—}20 (SEQ ID NO:21) according to the present invention is supported by 10 libraries. The number of libraries was determined as previously described. This segment can be found in the following transcript(s): HUMELAM1A_T1 (SEQ ID NO:10). Table 22 below describes the starting and ending position of this segment on each transcript.

TABLE 22
Segment location on transcripts

| | | Segment |
|---|---|---|
| | Segment | ending |
| Transcript name | starting position | position |
| HUMELAM1A_T1 (SEQ ID NO: 10) | 1573 | 1761 |

Segment cluster HUMELAM1A_node_{—}22 (SEQ ID NO:22) according to the present invention is supported by 10 libraries. The number of libraries was determined as previously described. This segment can be found in the following transcript(s): HUMELAM1A_T1 (SEQ ID NO:10). Table 23 below describes the starting and ending position of this segment on each transcript.

TABLE 23
Segment location on transcripts

| | | Segment |
|---|---|---|
| | Segment | ending |
| Transcript name | starting position | position |
| HUMELAM1A_T1 (SEQ ID NO: 10) | 1762 | 1938 |

Segment cluster HUMELAM1A_node_{—}33 (SEQ ID NO:23) according to the present invention is supported by 50 libraries. The number of libraries was determined as previously described. This segment can be found in the following transcript(s): HUMELAM1A_T1 (SEQ ID NO:10). Table 24 below describes the starting and ending position of this segment on each transcript.

TABLE 24
Segment location on transcripts

| | | Segment |
|---|---|---|
| | Segment | ending |
| Transcript name | starting position | position |
| HUMELAM1A_T1 (SEQ ID NO: 10) | 2142 | 4016 |

According to an optional embodiment of the present invention, short segments related to the above cluster are also provided. These segments are up to about 120 bp in length, and so are included in a separate description.

Segment cluster HUMELAM1A_node_{—}0 (SEQ ID NO:24) according to the present invention is supported by 14 libraries. The number of libraries was determined as previously described. This segment can be found in the following transcript(s): HUMELAM1A_T1 (SEQ ID NO:10), HUMELAM1A_T5 (SEQ ID NO:11 and HUMELAM1A_T6 (SEQ ID NO:12). Table 25 below describes the starting and ending position of this segment on each transcript.

TABLE 25
Segment location on transcripts

| | | Segment |
|---|---|---|
| | Segment | ending |
| Transcript name | starting position | position |
| HUMELAM1A_T1 (SEQ ID NO: 10) | 1 | 115 |
| HUMELAM1A_T5 (SEQ ID NO: 11) | 1 | 115 |
| HUMELAM1A_T6 (SEQ ID NO: 12) | 1 | 115 |

Segment cluster HUMELAM1A_node_{—}2 (SEQ ID NO:25) according to the present invention is supported by 15 libraries. The number of libraries was determined as previously described. This segment can be found in the following transcript(s): HUMELAM1A_T1 (SEQ ID NO:10), HUMELAM1A_T5 (SEQ ID NO:11) and HUMELAM1A_T6 (SEQ ID NO:12). Table 26 below describes the starting and ending position of this segment on each transcript.

TABLE 26
Segment location on transcripts

| | Segment | |
|---|---|---|
| | starting | Segment |
| Transcript name | position | ending position |
| HUMELAM1A_T1 (SEQ ID NO: 10) | 116 | 200 |
| HUMELAM1A_T5 (SEQ ID NO: 11) | 116 | 200 |
| HUMELAM1A_T6 (SEQ ID NO: 12) | 116 | 200 |

Segment cluster HUMELAM1A_node_{—}7 (SEQ ID NO:26) according to the present invention is supported by 13 libraries. The number of libraries was determined as previously described. This segment can be found in the following transcript(s): HUMELAM1A_T1 (SEQ ID NO:10), HUMELAM1A_T5 (SEQ ID NO:1) and HUMELAM1A_T6 (SEQ ID NO:12). Table 27 below describes the starting and ending position of this segment on each transcript.

TABLE 27
Segment location on transcripts

| | Segment | |
|---|---|---|
| | starting | Segment |
| Transcript name | position | ending position |
| HUMELAM1A_T1 (SEQ ID NO: 10) | 585 | 692 |
| HUMELAM1A_T5 (SEQ ID NO: 11) | 585 | 692 |
| HUMELAM1A_T6 (SEQ ID NO: 12) | 585 | 692 |

Segment cluster HUMELAM1A_node_{—}24 (SEQ ID NO:27) according to the present invention is supported by 5 libraries. The number of libraries was determined as previously described. This segment can be found in the following transcript(s): HUMELAM1A_T1 (SEQ ID NO:11). Table 28 below describes the starting and ending position of this segment on each transcript.

TABLE 28
Segment location on transcripts

| | Segment | |
|---|---|---|
| | starting | Segment |
| Transcript name | position | ending position |
| HUMELAM1A_T1 (SEQ ID NO: 10) | 1939 | 2046 |

Segment cluster HUMELAM1A_node_{—}26 (SEQ ID NO:28) according to the present invention can be found in the following transcript(s): HUMELAM1A_T1 (SEQ ID NO:10). Table 29 below describes the starting and ending position of this segment on each transcript.

TABLE 29
Segment location on transcripts

| | Segment | |
|---|---|---|
| | starting | Segment |
| Transcript name | position | ending position |
| HUMELAM1A_T1 (SEQ ID NO: 10) | 2047 | 2068 |

Segment cluster HUMELAM1A_node_{—}29 (SEQ ID NO:29) according to the present invention is supported by 8 libraries. The number of libraries was determined as previously described. This segment can be found in the following transcript(s): HUMELAM1A_T1 (SEQ ID NO:10). Table 30 below describes the starting and ending position of this segment on each transcript.

TABLE 30
Segment location on transcripts

| | Segment | |
|---|---|---|
| | starting | Segment |
| Transcript name | position | ending position |
| HUMELAM1A_T1 (SEQ ID NO: 10) | 2069 | 2141 |

Variant protein alignment to the previously known protein:

| Sequence name: LEM2_HUMAN (SEQ ID NO: 30) | | | |
|---|---|---|---|
| Sequence documentation: | | | |
| Alignment of: HUMELAM1A_P2 (SEQ ID NO: 31) × LEM2_HUMAN (SEQ ID NO: 30) .. | | | |
| Alignment segment 1/1: | | | |
| Quality: | 4376.00 | Escore: | 0 |
| Matching length: | 426 | Total length: | 426 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MIASQFLSALTLVLLIKESGAWSYNTSTEAMTYDEASAYCQQRYTHLVAI | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MIASQFLSALTLVLLIKESGAWSYNTSTEAMTYDEASAYCQQRYTHLVAI | 50 |
| | . . . . . | |
| 51 | QNKEEIEYLNSILSYSPSYYWIGIRKVNNVWVWVGTQKPLTEEAKNWAPG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QNKEEIEYLNSILSYSPSYYWIGIRKVNNVWVWVGTQKPLTEEAKNWAPG | 100 |
| | . . . . . | |
| 101 | EPNNRQKDEDCVEIYIKREKDVGMWNDERCSKKKLALCYTAACTNTSCSG | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | EPNNRQKDEDCVEIYIKREKDVGMWNDERCSKKKLALCYTAACTNTSCSG | 150 |
| | . . . . . | |
| 151 | HGECVETINNYTCKCDPGFSGLKCEQIVNCTALESPEHGSLVCSHPLGNF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | HGECVETINNYTCKCDPGFSGLKCEQIVNCTALESPEHGSLVCSHPLGNF | 200 |
| | . . . . . | |
| 201 | SYNSSCSISCDRGYLPSSMETMQCMSSGEWSAPIPACNVVECDAVTNPAN | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | SYNSSCSISCDRGYLPSSMETMQCMSSGEWSAPIPACNVVECDAVTNPAN | 250 |
| | . . . . . | |
| 251 | GFVECFQNPGSFPWNTTCTFDCEEGFELMGAQSLQCTSSGNWDNEKPTCK | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | GFVECFQNPGSFPWNTTCTFDCEEGFELMGAQSLQCTSSGNWDNEKPTCK | 300 |
| | . . . . . | |
| 301 | AVTCRAVRQPQNGSVRCSHSPAGEFTFKSSCNFTCEEGFMLQGPAQVECT | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | AVTCRAVRQPQNGSVRCSHSPAGEFTFKSSCNFTCEEGFMLQGPAQVECT | 350 |
| | . . . . . | |
| 351 | TQGQWTQQIPVCEAFQCTALSNPERGYMNCLPSASGSFRYGSSCEFSCEQ | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | TQGQWTQQIPVCEAFQCTALSNPERGYMNCLPSASGSFRYGSSCEFSCEQ | 400 |
| | . . | |
| 401 | GFVLKGSKRLQGGPTGEWDNEKPTCE | 426 |
| | \|\|\|\|\|\|\|\|\|\|\|\|\|\|\|\|\|\|\|\|\|\|\|\|\|\| | |
| 401 | GFVLKGSKRLQCGPTGEWDNEKPTCE | 426 |

| Sequence name: LEM2_HUMAN (SEQ ID NO: 30) | | | |
|---|---|---|---|
| Sequence documentation: | | | |
| Alignment of: HUMELAM1A_P2 (SEQ ID NO: 32) × LEM2_HUMAN (SEQ ID NO: 30) .. | | | |
| Alignment segment 1/1: | | | |
| Quality: | 2426.00 | Escore: | 0 |
| Matching length: | 238 | Total length: | 238 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MIASQFLSALTLVLLIKESGAWSYNTSTEAMTYDEASAYCQQRYTHLVAI | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MIASQFLSALTLVLLIKESGAWSYNTSTEANTYDEASAYCQQRYTHLVAI | 50 |
| | . . . . . | |
| 51 | QNKEEIEYLNSILSYSPSYYWIGIRKVNNVWVWVGTQKPLTEEAKNWAPG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QNKEEIEYLNSILSYSPSYYWIGIRKVNNVWVWVGTQKPLTEEAKNWAPG | 100 |
| | . . . . . | |
| 101 | EPNNRQKDEDCVEIYIKREKDVGMWNDERCSKKKLALCYTAACTNTSCSG | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | EPNNRQKDEDCVEIYIKREKDVGMWNDERCSKKKLALCYTAACTNTSCSG | 150 |
| | . . . . . | |
| 151 | HGECVETINNYTCKCDPGFSGLKCEQIVNCTALESPEHGSLVCSHPLGNF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | HGECVETINNYTCKCDPGFSGLKCEQIVNCTALESPEHGSLVCSHPLGNF | 200 |
| | . . . | |
| 201 | SYNSSCSISCDRGYLPSSMETMQCMSSGEWSAPIPACN | 238 |
| | \|\|\|\|\|\|\|\|\|\|\|\|\|\|\|\|\|\|\|\|\|\|\|\|\|\|\|\|\|\|\|\|\|\|\|\|\|\| | |
| 201 | SYNSSCSISCDRGYLPSSMETMQCMSSGEWSAPIPACN | 238 |

| Sequence name: LEM2_HUMAN | | | |
|---|---|---|---|
| Sequence documentation: | | | |
| Alignment of: HUMELAM1A_P2 (SEQ ID NO: 33) × LEM2_HUMAN (SEQ ID NO: 30) .. | | | |
| Alignment segment 1/1: | | | |
| Quality: | 1786.00 | Escore: | 0 |
| Matching length: | 176 | Total length: | 176 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MIASQFLSALTLVLLIKESGAWSYNTSTEAMTYDEASAYCQQRYTHLVAI | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MIASQFLSALTLVLLIKESGAWSYNTSTEAMTYDEASAYCQQRYTHLVAI | 50 |
| | . . . . . | |
| 51 | QNKEEIEYLNSILSYSPSYYWIGIRKVNNVWVWVGTQKPLTEEAKNWAPG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QNKEEIEYLNSILSYSPSYYWIGIRKVNNVWVWVGTQKPLTEEAKNWAPG | 100 |
| | . . . . . | |
| 101 | EPNNRQKDEDCVEIYIKREKDVGMWNDERCSKKKLALCYTAACTNTSCSG | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | EPNNRQKDEDCVEIYIKREKDVGMWNDERCSKKKLALCYTAACTNTSCSG | 150 |
| | . . | |
| 151 | HGECVETINNYTCKCDPGFSGLKCEQ | 176 |
| | \|\|\|\|\|\|\|\|\|\|\|\|\|\|\|\|\|\|\|\|\|\|\|\|\|\| | |
| 151 | HGECVETINNYTCKCDPGFSGLKCEQ | 176 |

### Description for Cluster HUMHPA1B

Cluster HUMHPA1B features 13 transcript(s) and 84 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HUMHPA1B_PEA_1_T1 | 34 |
| HUMHPA1B_PEA_1_T4 | 35 |
| HUMHPA1B_PEA_1_T6 | 36 |
| HUMHPA1B_PEA_1_T7 | 37 |
| HUMHPA1B_PEA_1_T12 | 38 |
| HUMHPA1B_PEA_1_T16 | 39 |
| HUMHPA1B_PEA_1_T19 | 40 |
| HUMHPA1B_PEA_1_T20 | 41 |
| HUMHPA1B_PEA_1_T27 | 42 |
| HUMHPA1B_PEA_1_T29 | 43 |
| HUMHPA1B_PEA_1_T55 | 44 |
| HUMHPA1B_PEA_1_T56 | 45 |
| HUMHPA1B_PEA_1_T59 | 46 |

TABLE 2
Segments of interest

| Segment Name | Sequence ID No. |
|---|---|
| HUMHPA1B_PEA_1_node_20 | 47 |
| HUMHPA1B_PEA_1_node_25 | 48 |
| HUMHPA1B_PEA_1_node_28 | 49 |
| HUMHPA1B_PEA_1_node_35 | 50 |
| HUMHPA1B_PEA_1_node_88 | 51 |
| HUMHPA1B_PEA_1_node_0 | 52 |
| HUMHPA1B_PEA_1_node_1 | 53 |
| HUMHPA1B_PEA_1_node_3 | 54 |
| HUMHPA1B_PEA_1_node_4 | 55 |
| HUMHPA1B_PEA_1_node_5 | 56 |
| HUMHPA1B_PEA_1_node_6 | 57 |
| HUMHPA1B_PEA_1_node_7 | 58 |
| HUMHPA1B_PEA_1_node_10 | 59 |
| HUMHPA1B_PEA_1_node_11 | 60 |
| HUMHPA1B_PEA_1_node_12 | 61 |
| HUMHPA1B_PEA_1_node_13 | 62 |
| HUMHPA1B_PEA_1_node_14 | 63 |
| HUMHPA1B_PEA_1_node_15 | 64 |
| HUMHPA1B_PEA_1_node_16 | 65 |
| HUMHPA1B_PEA_1_node_17 | 66 |
| HUMHPA1B_PEA_1_node_18 | 67 |
| HUMHPA1B_PEA_1_node_19 | 68 |
| HUMHPA1B_PEA_1_node_21 | 69 |
| HUMHPA1B_PEA_1_node_22 | 70 |
| HUMHPA1B_PEA_1_node_23 | 71 |
| HUMHPA1B_PEA_1_node_24 | 72 |
| HUMHPA1B_PEA_1_node_27 | 73 |
| HUMHPA1B_PEA_1_node_29 | 74 |
| HUMHPA1B_PEA_1_node_30 | 75 |
| HUMHPA1B_PEA_1_node_31 | 76 |
| HUMHPA1B_PEA_1_node_32 | 77 |
| HUMHPA1B_PEA_1_node_33 | 78 |
| HUMHPA1B_PEA_1_node_34 | 79 |
| HUMHPA1B_PEA_1_node_36 | 80 |
| HUMHPA1B_PEA_1_node_37 | 81 |
| HUMHPA1B_PEA_1_node_38 | 82 |
| HUMHPA1B_PEA_1_node_39 | 83 |
| HUMHPA1B_PEA_1_node_40 | 84 |
| HUMHPA1B_PEA_1_node_41 | 85 |
| HUMHPA1B_PEA_1_node_42 | 86 |
| HUMHPA1B_PEA_1_node_43 | 87 |
| HUMHPA1B_PEA_1_node_44 | 88 |
| HUMHPA1B_PEA_1_node_45 | 89 |
| HUMHPA1B_PEA_1_node_46 | 90 |
| HUMHPA1B_PEA_1_node_47 | 91 |
| HUMHPA1B_PEA_1_node_48 | 92 |
| HUMHPA1B_PEA_1_node_49 | 93 |
| HUMHPA1B_PEA_1_node_50 | 94 |
| HUMHPA1B_PEA_1_node_51 | 95 |
| HUMHPA1B_PEA_1_node_52 | 96 |
| HUMHPA1B_PEA_1_node_53 | 97 |
| HUMHPA1B_PEA_1_node_54 | 98 |
| HUMHPA1B_PEA_1_node_55 | 99 |
| HUMHPA1B_PEA_1_node_56 | 100 |
| HUMHPA1B_PEA_1_node_57 | 101 |
| HUMHPA1B_PEA_1_node_58 | 102 |
| HUMHPA1B_PEA_1_node_59 | 103 |
| HUMHPA1B_PEA_1_node_60 | 104 |
| HUMHPA1B_PEA_1_node_61 | 105 |
| HUMHPA1B_PEA_1_node_62 | 106 |
| HUMHPA1B_PEA_1_node_63 | 107 |
| HUMHPA1B_PEA_1_node_64 | 108 |
| HUMHPA1B_PEA_1_node_65 | 109 |
| HUMHPA1B_PEA_1_node_66 | 110 |
| HUMHPA1B_PEA_1_node_67 | 111 |
| HUMHPA1B_PEA_1_node_69 | 112 |
| HUMHPA1B_PEA_1_node_70 | 113 |
| HUMHPA1B_PEA_1_node_71 | 114 |
| HUMHPA1B_PEA_1_node_72 | 115 |
| HUMHPA1B_PEA_1_node_73 | 116 |
| HUMHPA1B_PEA_1_node_74 | 117 |
| HUMHPA1B_PEA_1_node_75 | 118 |
| HUMHPA1B_PEA_1_node_76 | 119 |
| HUMHPA1B_PEA_1_node_77 | 120 |
| HUMHPA1B_PEA_1_node_78 | 121 |
| HUMHPA1B_PEA_1_node_79 | 122 |
| HUMHPA1B_PEA_1_node_80 | 123 |
| HUMHPA1B_PEA_1_node_81 | 124 |
| HUMHPA1B_PEA_1_node_82 | 125 |
| HUMHPA1B_PEA_1_node_83 | 126 |
| HUMHPA1B_PEA_1_node_84 | 127 |
| HUMHPA1B_PEA_1_node_85 | 128 |
| HUMHPA1B_PEA_1_node_86 | 129 |
| HUMHPA1B_PEA_1_node_87 | 130 |

TABLE 3
Proteins of interest

| | Sequence | |
|---|---|---|
| Protein Name | ID No. | Corresponding Transcript(s) |
| HUMHPA1B_PEA_1_P61 | 133 | HUMHPA1B_PEA_1_T1 |
| | | (SEQ ID NO: 34) |
| HUMHPA1B_PEA_1_P62 | 134 | HUMHPA1B_PEA_1_T4 |
| | | (SEQ ID NO: 35) |
| HUMHPA1B_PEA_1_P64 | 135 | HUMHPA1B_PEA_1_T6 |
| | | (SEQ ID NO: 36) |
| HUMHPA1B_PEA_1_P65 | 136 | HUMHPA1B_PEA_1_T7 |
| | | (SEQ ID NO: 37) |
| HUMHPA1B_PEA_1_P68 | 137 | HUMHPA1B_PEA_1_T12 |
| | | (SEQ ID NO: 38) |
| HUMHPA1B_PEA_1_P72 | 138 | HUMHPA1B_PEA_1_T16 |
| | | (SEQ ID NO: 39) |
| HUMHPA1B_PEA_1_P75 | 139 | HUMHPA1B_PEA_1_T19 |
| | | (SEQ ID NO: 40) |
| HUMHPA1B_PEA_1_P76 | 140 | HUMHPA1B_PEA_1_T20 |
| | | (SEQ ID NO: 41) |
| HUMHPA1B_PEA_1_P81 | 141 | HUMHPA1B_PEA_1_T27 |
| | | (SEQ ID NO: 42) |
| HUMHPA1B_PEA_1_P83 | 142 | HUMHPA1B_PEA_1_T29 |
| | | (SEQ ID NO: 43) |
| HUMHPA1B_PEA_1_P106 | 143 | HUMHPA1B_PEA_1_T55 |
| | | (SEQ ID NO: 44) |
| HUMHPA1B_PEA_1_P107 | 144 | HUMHPA1B_PEA_1_T56 |
| | | (SEQ ID NO: 45) |
| HUMHPA1B_PEA_1_P115 | 145 | HUMHPA1B_PEA_1_T59 |
| | | (SEQ ID NO: 46) |

These sequences are variants of the known protein Haptoglobin precursor (SEQ ID NO:131) (SwissProt accession identifier HPT_HUMAN), referred to herein as the previously known protein.

Protein Haptoglobin precursor (SEQ ID NO:131) is known or believed to have the following function(s): haptoglobin combines with free plasma hemoglobin, preventing loss of iron through the kidneys and protecting the kidneys from damage by hemoglobin, while making the hemoglobin accessible to degradative enzymes. The sequence for protein Haptoglobin precursor is given at the end of the application, as “Haptoglobin precursor amino acid sequence” (SEQ ID NO:131). Known polymorphisms for this sequence are as shown in Table 4.

TABLE 4
Amino acid mutations for Known Protein

| SNP position(s) on | |
|---|---|
| amino acid sequence | Comment |
| 29-87 | Missing (in allele HP*1F and allele HP*1S). |
| | /FTId = VAR_017112. |
| 193 | N -> D (in allele HP*1F). /FTId = VAR_005294. |
| 194 | E -> K (in allele HP*1F). /FTId = VAR_017113. |
| 397 | D -> H (in dbSNP: 12646). /FTId = VAR_017114. |
| 70 | D -> N |
| 90 | D -> E |

Protein Haptoglobin precursor (SEQ ID NO:131) localization is believed to be Secreted.

Endometriotic lesions synthesize and secrete a unique form of haptoglobin (endometriosis protein-I) that is up-regulated by IL-6 (Sharpe-Timms et al, Fertil Steril. 2002 October; 78(4):810-9). Variants of this cluster are suitable as diagnostic markers for endometriosis.

The following GO Annotation(s) apply to the previously known protein. The following annotation(s) were found: defense response, which are annotation(s) related to Biological Process.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster HUMHPA1B features 13 transcript(s), which were listed in Table 1 above. These transcript(s) encode for protein(s) which are variant(s) of protein Haptoglobin precursor (SEQ ID NO:131). A description of each variant protein according to the present invention is now provided.

Variant protein HUMHPA1B_PEA_{—}1_P61 (SEQ ID NO:133) according to the present invention has an amino acid sequence as given at the end of the application; it is encoded by transcript(s) HUMHPA1B_PEA_{—}1_T1 (SEQ ID NO:34). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P61 (SEQ ID NO:133) and HPT_HUMAN (SEQ ID NO:131):

1. An isolated chimeric polypeptide encoding for HUMHPA1B_PEA_{—}1_P61 (SEQ ID NO:133), comprising a first amino acid sequence being at least 90% homologous to MSALGAVIALLLWGQLFAVDSGNDVTDI corresponding to amino acids 1-28 of HPT_HUMAN (SEQ ID NO:131), which also corresponds to amino acids 1-28 of HUMHPA1B_PEA_{—}1_P61 (SEQ ID NO:133), and a second amino acid sequence being at least 90% homologous to ADDGCPKPPEIAHGYVEHSVRYQCKNYYKLRTEGDGVYTLNNEKQWINKAVGDKLPE CEAVCGKPKNPANPVQRILGGHLDAKGSFPWQAKMVSHHNLTTGATLINEQWLLTTA KNLFLNHSENATAKDIAPTLTLYVGKKQLVEIEKVVLHPNYSQVDIGLIKLKQKVSVNE RVMPICLPSKDYAEVGRVGYVSGWGRNANFKFTDHLKYVMLPVADQDQCIRHYEGST VPEKKTPKSPVGVQPILNEHTFCAGMSKYQEDTCYGDAGSAFAVHDLEEDTWYATGIL SFDKSCAVAEYGVYVKVTSIQDWVQKTIAEN corresponding to amino acids 88-406 of HPT_HUMAN (SEQ ID NO:131), which also corresponds to amino acids 29-347 of HUMHPA1B_PEA_{—}1_P61 (SEQ ID NO:133), wherein said first amino acid sequence and second amino acid sequence are contiguous and in a sequential order.

2. An isolated chimeric polypeptide encoding for an edge portion of HUMHPA1B_PEA_{—}1_P61 (SEQ ID NO:133), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IA, having a structure as follows: a sequence starting from any of amino acid numbers 28−x to 28; and ending at any of amino acid numbers 29+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMHPA1B_PEA_{—}1_P61 (SEQ ID NO:133) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUMHPA1B_PEA_{—}1_P61 (SEQ ID NO:133) Sequence provides support for the deduced sequence of this variant protein according to the present invention).

TABLE 7
Amino acid mutations

| SNP position(s) on amino acid | | Previously |
|---|---|---|
| sequence | Alternative amino acid(s) | known SNP? |
| 8 | I -> | No |
| 38 | E -> D | No |
| 71 | E -> G | No |
| 71 | E -> K | No |
| 108 | L -> V | No |
| 136 | Q -> | No |
| 162 | L -> V | No |
| 176 | K -> | No |
| 184 | S -> P | Yes |
| 194 | K -> | No |
| 242 | L -> P | No |
| 260 | P -> L | No |
| 296 | A -> | No |

The glycosylation sites of variant protein HUMHPA1B_PEA_{—}1_P61 (SEQ ID NO:133), as compared to the known protein Haptoglobin precursor (SEQ ID NO:131), are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on known amino | | Position in |
|---|---|---|
| acid sequence | Present in variant protein? | variant protein? |
| 207 | yes | 148 |
| 241 | yes | 182 |
| 184 | yes | 125 |
| 211 | yes | 152 |

Variant protein HUMHPA1B_PEA_{—}1_P61 (SEQ ID NO:133) is encoded by the following transcript(s): HUMHPA1B_PEA_{—}1_T1 (SEQ ID NO:34), for which the sequence(s) is/are given at the end of the application. The coding portion of transcript HUMHPA1B_PEA_{—}1_T1 (SEQ ID NO:34) is shown in bold; this coding portion starts at position 68 and ends at position 1108. The transcript also has the following SNPs as listed in Table 9 (given according to their position on the nucleotide sequence, with the alternative nucleic acid listed; the last column indicates whether the SNP is known or not; the presence of known SNPs in variant protein HUMHPA11B_PEA_{—}1_P61 (SEQ ID NO:133) Sequence provides support for the deduced sequence of this variant protein according to the present invention).

TABLE 9
Nucleic acid SNPs

| SNP position on nucleotide | | Previously |
|---|---|---|
| sequence | Alternative nucleic acid | known SNP? |
| 40 | T -> G | No |
| 77 | C -> T | No |
| 90 | T -> | No |
| 181 | G -> T | No |
| 262 | G -> A | Yes |
| 278 | G -> A | No |
| 279 | A -> G | No |
| 304 | -> G | No |
| 337 | -> G | No |
| 389 | C -> G | No |
| 454 | T -> C | Yes |
| 454 | T -> G | Yes |
| 474 | A -> | No |
| 547 | T -> C | No |
| 550 | -> G | No |
| 551 | T -> G | No |
| 589 | T -> C | No |
| 595 | G -> | No |
| 617 | T -> C | Yes |
| 622 | G -> A | No |
| 647 | A -> | No |
| 694 | T -> A | No |
| 792 | T -> C | No |
| 826 | T -> C | No |
| 846 | C -> T | No |
| 886 | -> C | No |
| 913 | T -> C | No |
| 929 | -> C | No |
| 955 | G -> | No |
| 955 | G -> C | No |
| 978 | -> C | No |
| 993 | -> C | No |
| 1074 | -> C | No |
| 1141 | A -> C | No |
| 1142 | A -> G | No |
| 1235 | -> G | No |
| 1235 | -> T | No |

Variant protein HUMHPA1B_PEA_{—}1_P62 (SEQ ID NO:134) according to the present invention has an amino acid sequence as given at the end of the application; it is encoded by transcript(s) HUMHPA1B_PEA_{—}1_T4 (SEQ ID NO:35). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P62 (SEQ ID NO:134) and HPT_HUMAN (SEQ ID NO:131):

1. An isolated chimeric polypeptide encoding for HUMHPA1B_PEA_{—}1_P62 (SEQ ID NO:134), comprising a first amino acid sequence being at least 90% homologous to MSALGAVIALLLWGQLFAVDSGNDVTDIADDGCPKPPEIAHGYVEHSVRYQCKNYYK LRTEGDG corresponding to amino acids 1-64 of HPT_HUMAN (SEQ ID NO:131), which also corresponds to amino acids 1-64 of HUMHPA1B_PEA_{—}1_P62 (SEQ ID NO:134), and a second amino acid sequence being at least 70%, optionally at least 80%, preferably at least 85%, more preferably at least 90% and most preferably at least 95% homologous to a polypeptide having the sequence KMWTTVSMPYIQPPSLTFP (SEQ ID NO:495) corresponding to amino acids 65-83 of HUMHPA1B_PEA_{—}1_P62 (SEQ ID NO:134), wherein said first amino acid sequence and second amino acid sequence are contiguous and in a sequential order.

2. An isolated polypeptide encoding for a tail of HUMHPA1B_PEA_{—}1_P62 (SEQ ID NO:134), comprising a polypeptide being at least 70%, optionally at least about 80%, preferably at least about 85%, more preferably at least about 90% and most preferably at least about 95% homologous to the sequence KMWTTVSMPYIQPPSLTFP (SEQ ID NO:495) in HUMHPA1B_PEA_{—}1_P62(SEQ ID NO:134).

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P62 (SEQ ID NO:134) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HUMHPA1B_PEA_{—}1_P62 (SEQ ID NO:134) Sequence provides support for the deduced sequence of this variant protein according to the present invention).

TABLE 10
Amino acid mutations

| SNP position(s) on amino | Alternative | |
|---|---|---|
| acid sequence | amino acid(s) | Previously known SNP? |
| 8 | I -> | No |
| 38 | E -> D | No |

The glycosylation sites of variant protein HUMHPA1B_PEA_{—}1_P62 (SEQ ID NO:134), as compared to the known protein Haptoglobin precursor (SEQ ID NO:131), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | |
|---|---|
| acid sequence | Present in variant protein? |
| 207 | no |
| 241 | no |
| 184 | no |
| 211 | no |

Variant protein HUMHPA1B_PEA_{—}1_P62 (SEQ ID NO:134) is encoded by the following transcript(s): HUMHPA1B_PEA_{—}1_T4 (SEQ ID NO:35), for which the sequence(s) is/are given at the end of the application. The coding portion of transcript HUMHPA1B_PEA_{—}1_T4 (SEQ ID NO:35) is shown in hold; this coding portion starts at position 68 and ends at position 316. The transcript also has the following SNPs as listed in Table 12 (given according to their position on the nucleotide sequence, with the alternative nucleic acid listed; the last column indicates whether the SNP is known or not; the presence of known SNPs in variant protein HUMHPA1B_PEA_{—}1_P62 (SEQ ID NO:134) Sequence provides support for the deduced sequence of this variant protein according to the present invention).

TABLE 12
Nucleic acid SNPs

| SNP position on nucleotide | | Previously |
|---|---|---|
| sequence | Alternative nucleic acid | known SNP? |
| 40 | T -> G | No |
| 77 | C -> T | No |
| 90 | T -> | No |
| 181 | G -> T | No |
| 512 | G -> C | No |
| 781 | T -> C | Yes |
| 798 | G -> C | Yes |
| 879 | A -> G | Yes |
| 1063 | T -> | No |
| 1124 | A -> G | Yes |
| 1173 | A -> G | No |
| 1199 | G -> A | Yes |
| 1215 | G -> A | No |
| 1216 | A -> G | No |
| 1241 | -> G | No |
| 1274 | -> G | No |
| 1326 | C -> G | No |
| 1391 | T -> C | Yes |
| 1391 | T -> G | Yes |
| 1411 | A -> | No |
| 1484 | T -> C | No |
| 1487 | -> G | No |
| 1488 | T -> G | No |
| 1526 | T -> C | No |
| 1532 | G -> | No |
| 1554 | T -> C | Yes |
| 1559 | G -> A | No |
| 1584 | A -> | No |
| 1631 | T -> A | No |
| 1729 | T -> C | No |
| 1763 | T -> C | No |
| 1783 | C -> T | No |
| 1823 | -> C | No |
| 1850 | T -> C | No |
| 1866 | -> C | No |
| 1892 | G -> | No |
| 1892 | G -> C | No |
| 1915 | -> C | No |
| 1930 | -> C | No |
| 2011 | -> C | No |
| 2078 | A -> C | No |
| 2079 | A -> G | No |
| 2172 | -> G | No |
| 2172 | -> T | No |

Variant protein HUMHPA1B_PEA_{—}1_P64 (SEQ ID NO:135) according to the present invention has an amino acid sequence as given at the end of the application; it is encoded by transcript(s) HUMHPA1B_PEA_{—}1_T6 (SEQ ID NO:36). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P64 (SEQ ID NO:135) and HPT_HUMAN (SEQ ID NO:131):

1. An isolated chimeric polypeptide encoding for HUMHPA1B_PEA_{—}1_P64 (SEQ ID NO:135), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWfNKAVGDKLPECEADDGCPKPPEIAHGYVEHSVRYQCKNY YKLRTEGDG corresponding to amino acids 1-123 of HPT_HUMAN (SEQ ID NO:131), which also corresponds to amino acids 1-123 of HUMHPA1B_PEA_{—}1_P64 (SEQ ID NO:135), and a second amino acid sequence being at least 70%, optionally at least 80%, preferably at least 85%, more preferably at least 90% and most preferably at least 95% homologous to a polypeptide having the sequence KMWTTVSMPYIQPPSLTFP (SEQ ID NO:495) corresponding to amino acids 124-142 of HUMHPA1B_PEA_{—}1_P64 (SEQ ID NO:135), wherein said first amino acid sequence and second amino acid sequence are contiguous and in a sequential order.

2. An isolated polypeptide encoding for a tail of HUMHPA1B_PEA_{—}1_P64 (SEQ ID NO:135), comprising a polypeptide being at least 70%, optionally at least about 80%, preferably at least about 85%, more preferably at least about 90% and most preferably at least about 95% homologous to the sequence KMWTTVSMPYIQPPSLTFP (SEQ ID NO:495) in HUMHPA1B_PEA_{—}1_P64 (SEQ ID NO:135).

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P64 (SEQ ID NO:135) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HUMHPA1B_PEA_{—}1_P64 (SEQ ID NO:135) Sequence provides support for the deduced sequence of this variant protein according to the present invention).

TABLE 13
Amino acid mutations

| SNP position(s) on amino acid | | Previously |
|---|---|---|
| sequence | Alternative amino acid(s) | known SNP? |
| 8 | I -> | No |
| 38 | E -> D | No |
| 79 | V -> | No |
| 116 | K -> E | No |

The glycosylation sites of variant protein HUMHPA1B_PEA_{—}1_P64 (SEQ ID NO:135), as compared to the known protein Haptoglobin precursor (SEQ ID NO:131), are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known amino | |
|---|---|
| acid sequence | Present in variant protein? |
| 207 | no |
| 241 | no |
| 184 | no |
| 211 | no |

Variant protein HUMHPA1B_PEA_{—}1_P64 (SEQ ID NO:135) is encoded by the following transcript(s): HUMHPA1B_PEA_{—}1_T6 (SEQ ID NO:36), for which the sequence(s) is/are given at the end of the application. The coding portion of transcript HUMHPA1B_PEA_{—}1_T6 (SEQ ID NO:36) is shown in bold; this coding portion starts at position 68 and ends at position 493. The transcript also has the following SNPs as listed in Table 15 (given according to their position on the nucleotide sequence, with the alternative nucleic acid listed; the last column indicates whether the SNP is known or not; the presence of known SNPs in variant protein HUMHPA1B_PEA_L_P64 (SEQ ID NO:135) Sequence provides support for the deduced sequence of this variant protein according to the present invention).

TABLE 15
Nucleic acid SNPs

| SNP position on nucleotide | | Previously |
|---|---|---|
| sequence | Alternative nucleic acid | known SNP? |
| 40 | T -> G | No |
| 77 | C -> T | No |
| 90 | T -> | No |
| 181 | G -> T | No |
| 303 | T -> | No |
| 364 | A -> G | Yes |
| 413 | A -> G | No |
| 570 | C -> G | No |
| 689 | G -> C | No |
| 1060 | A -> T | Yes |
| 1103 | C -> T | Yes |
| 1109 | G -> A | Yes |
| 1118 | T -> C | Yes |
| 1180 | C -> T | Yes |
| 1197 | G -> A | Yes |
| 1213 | G -> A | No |
| 1214 | A -> G | No |
| 1239 | -> G | No |
| 1272 | -> G | No |
| 1324 | C -> G | No |
| 1389 | T -> C | Yes |
| 1389 | T -> G | Yes |
| 1409 | A -> | No |
| 1482 | T -> C | No |
| 1485 | -> G | No |
| 1486 | T -> G | No |
| 1524 | T -> C | No |
| 1530 | G -> | No |
| 1552 | T -> C | Yes |
| 1557 | G -> A | No |
| 1582 | A -> | No |
| 1629 | T -> A | No |
| 1727 | T -> C | No |
| 1761 | T -> C | No |
| 1781 | C -> T | No |
| 1821 | -> C | No |
| 1848 | T -> C | No |
| 1864 | -> C | No |
| 1890 | G -> | No |
| 1890 | G -> C | No |
| 1913 | -> C | No |
| 1928 | -> C | No |
| 2009 | -> C | No |
| 2076 | A -> C | No |
| 2077 | A -> G | No |
| 2170 | -> G | No |
| 2170 | -> T | No |

Variant protein HUMHPA1B_PEA_{—}1_P65 (SEQ ID NO:136) according to the present ion has an amino acid sequence as given at the end of the application; it is encoded by transcript(s) HUMHPA1B_PEA_{—}1_T7 (SEQ ID NO:37). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P65 (SEQ ID NO:136) and HPT_HUMAN (SEQ ID NO:131):

1. An isolated chimeric polypeptide encoding for HUMHPA1B_PEA_{—}1_P65 (SEQ ID NO:136), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEADDGCPKPPEIAHGYVEHSVRYQCKNY YKLRTEGDGVYTLNNEKQWINKAVGDKLPECEA corresponding to amino acids 1-147 of HPT_HUMAN (SEQ ID NO:131), which also corresponds to amino acids 1-147 of HUMHPA1B_PEA_{—}1_P65 (SEQ ID NO:136), and a second amino acid sequence being at least 70%, optionally at least 80%, preferably at least 85%, more preferably at least 90% and most preferably at least 95% homologous to a polypeptide having the sequence GGC corresponding to amino acids 148-150 of HUMHPA1B_PEA_{—}1_P65 (SEQ ID NO:136), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P65 (SEQ ID NO:136) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein HUMHPA1B_PEA_{—}1_P65 (SEQ ID NO:136) Sequence provides support for the deduced sequence of this variant protein according to the present invention).

TABLE 16
Amino acid mutations

| SNP position(s) on amino acid | | Previously |
|---|---|---|
| sequence | Alternative amino acid(s) | known SNP? |
| 8 | I -> | No |
| 38 | E -> D | No |
| 79 | V -> | No |
| 116 | K -> E | No |
| 130 | E -> G | No |
| 130 | E -> K | No |

The glycosylation sites of variant protein HUMHPA1B_PEA_{—}1_P65 (SEQ ID NO:136), as compared to the known protein Haptoglobin precursor (SEQ ID NO:131), are described in Table 17 (given according to their position(s) on the amino acid sequence in the first column; the second column indicates whether the glycosylation site is present in the variant protein; and the last column indicates whether the position is different on the variant protein).

TABLE 17
Glycosylation site(s)

| Position(s) on known amino | |
|---|---|
| acid sequence | Present in variant protein? |
| 207 | no |
| 241 | no |
| 184 | no |
| 211 | no |

Variant protein HUMHPA1B_PEA_{—}1_P65 (SEQ ID NO:136) is encoded by the following transcript(s): HUMHPA1B_PEA_{—}1_T7 (SEQ ID NO:37), for which the sequence(s) is/are given at the end of the application. The coding portion of transcript HUMHPA1B_PEA_{—}1_T7 (SEQ ID NO:37) is shown in bold; this coding portion starts at position 68 and ends at position 517. The transcript also has the following SNPs as listed in Table 18 (given according to their position on the nucleotide sequence, with the alternative nucleic acid listed; the last column indicates whether the SNP is known or not; the presence of known SNPs in variant protein HUMHPA1B_PEA_{—}1_P65 (SEQ ID NO:136) Sequence provides support for the deduced sequence of this variant protein according to the present invention).

TABLE 18
Nucleic acid SNPs

| SNP position on | | |
|---|---|---|
| nucleotide sequence | Alternative nucleic acid | Previously known SNP? |
| 40 | T -> G | No |
| 77 | C -> T | No |
| 90 | T -> | No |
| 181 | G -> T | No |
| 303 | T -> | No |
| 364 | A -> G | Yes |
| 413 | A -> G | No |
| 439 | G -> A | Yes |
| 455 | G -> A | No |
| 456 | A -> G | No |
| 481 | -> G | No |
| 556 | C -> A | Yes |
| 730 | T -> C | Yes |
| 751 | T -> C | Yes |
| 945 | A -> C | Yes |
| 956 | G -> A | Yes |
| 1312 | G -> A | Yes |
| 1332 | T -> C | Yes |
| 1437 | -> G | No |
| 1489 | C -> G | No |
| 1554 | T -> C | Yes |
| 1554 | T -> G | Yes |
| 1574 | A -> | No |
| 1647 | T -> C | No |
| 1650 | -> G | No |
| 1651 | T -> G | No |
| 1689 | T -> C | No |
| 1695 | G -> | No |
| 1717 | T -> C | Yes |
| 1722 | G -> A | No |
| 1747 | A -> | No |
| 1794 | T -> A | No |
| 1892 | T -> C | No |
| 1926 | T -> C | No |
| 1946 | C -> T | No |
| 1986 | -> C | No |
| 2013 | T -> C | No |
| 2029 | -> C | No |
| 2055 | G -> | No |
| 2055 | G -> C | No |
| 2078 | -> C | No |
| 2093 | -> C | No |
| 2174 | -> C | No |
| 2241 | A -> C | No |
| 2242 | A -> G | No |
| 2335 | -> G | No |
| 2335 | -> T | No |

Variant protein HUMHPA1B_PEA_{—}1_P68 (SEQ ID NO:137) according to the present invention has an amino acid sequence as given at the end of the application; it is encoded by transcript(s) HUMHPA1B_PEA_{—}1_T12 (SEQ ID NO:38). An alignment is given to the known (Haptoglobin precursor (SEQ ID NO:131)) at the end of the application. One or more ents to one or more previously published protein sequences are given at the end of the application. A brief description of the relationship of the variant protein according to the present invention to each such aligned protein is as follows:

Comparison report between HUMHPA1B_PEA_{—}1_P68 (SEQ ID NO:137) and HPT_HUMAN (SEQ ID NO:131):

1. An isolated chimeric polypeptide encoding for HUMHPA1B_PEA_{—}1_P68 (SEQ ID NO:137), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDK corresponding to amino acids 1-71 of HPT_HUMAN (SEQ ID NO:131), which also corresponds to amino acids 1-71 of HUMHPA1B_PEA_{—}1_P68 (SEQ ID NO:137), and a second amino acid sequence being at least 90% homologous to KQWINKAVGDKLPECEAVCGKPKNPANPVQRILGGHLDAKGSFPWQAKMVSHHNLTT GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 131-406 of HPT_HUMAN (SEQ ID NO:131), which also corresponds to amino acids 72-347 of HUMHPA1B_PEA_{—}1_P68 (SEQ ID NO:137), wherein said first amino acid sequence and second amino acid sequence are contiguous and in a sequential order.

2. An isolated chimeric polypeptide encoding for an edge portion of HUMHPA1B_PEA_{—}1_P68 (SEQ ID NO:137), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KK, having a structure as follows: a sequence starting from any of amino acid numbers 71−x to 71; and ending at any of amino acid numbers 72+((n−2)−x), in which x varies from 0 to n-2.

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P68 (SEQ ID NO:137) also has the following non-silent SNPs (Single Nucleotide Polymorphisms) as listed in Table 19, (given according to their position(s) on the amino acid sequence, with the alternative amino acid(s) listed; the last column indicates whether the SNP is known or not; the presence of known SNPs in variant protein HUMHPA1B_PEA_{—}1_P68 (SEQ ID NO:137) Sequence provides support for the deduced sequence of this variant protein according to the present invention).

TABLE 19
Amino acid mutations

| SNP position(s) on | | |
|---|---|---|
| amino acid sequence | Alternative amino acid(s) | Previously known SNP? |
| 8 | I -> | No |
| 38 | E -> D | No |
| 79 | V -> | No |
| 108 | L -> V | No |
| 136 | Q -> | No |
| 162 | L -> V | No |
| 176 | K -> | No |
| 184 | S -> P | Yes |
| 194 | K -> | No |
| 242 | L -> P | No |
| 260 | P -> L | No |
| 296 | A -> | No |

The glycosylation sites of variant protein HUMHPA1B_PEA_{—}1_P68 (SEQ ID NO:137), as compared to the known protein Haptoglobin precursor (SEQ ID NO:131), are described in Table 20 (given according to their position(s) on the amino acid sequence in the first column; the second column indicates whether the glycosylation site is present in the variant protein; and the last column indicates whether the position is different on the variant protein).

TABLE 20
Glycosylation site(s)

| Position(s) on | | |
|---|---|---|
| known amino | | |
| acid sequence | Present in variant protein? | Position in variant protein? |
| 207 | yes | 148 |
| 241 | yes | 182 |
| 184 | yes | 125 |
| 211 | yes | 152 |

Variant protein HUMHPA1B_PEA_{—}1_P68 (SEQ ID NO:137) is encoded by the following transcript(s): HUMHPA1B_PEA_{—}1_T12 (SEQ ID NO:38, for which the sequence(s) is/are given at the end of the application. The coding portion of transcript HUMHPA1B_PEA_{—}1_T12 SEQ ID NO:38) is shown in bold; this coding portion starts at position 68 and ends at position 1108. The transcript also has the following SNPs as listed in Table 21 (given according to their position on the nucleotide sequence, with the alternative nucleic acid listed; the last column indicates whether the SNP is known or not; the presence of known SNPs in variant protein HUMHPA1B_PEA_{—}1_P68 (SEQ ID NO:137) Sequence provides support for the deduced sequence of this variant protein according to the present invention).

TABLE 21
Nucleic acid SNPs

| SNP position on | | |
|---|---|---|
| nucleotide sequence | Alternative nucleic acid | Previously known SNP? |
| 40 | T -> G | No |
| 77 | C -> T | No |
| 90 | T -> | No |
| 181 | G -> T | No |
| 303 | T -> | No |
| 337 | -> G | No |
| 389 | C -> G | No |
| 454 | T -> C | Yes |
| 454 | T -> G | Yes |
| 474 | A -> | No |
| 547 | T -> C | No |
| 550 | -> G | No |
| 551 | T -> G | No |
| 589 | T -> C | No |
| 595 | G -> | No |
| 617 | T -> C | Yes |
| 622 | G -> A | No |
| 647 | A -> | No |
| 694 | T -> A | No |
| 792 | T -> C | No |
| 826 | T -> C | No |
| 846 | C -> T | No |
| 886 | -> C | No |
| 913 | T -> C | No |
| 929 | -> C | No |
| 955 | G -> | No |
| 955 | G -> C | No |
| 978 | -> C | No |
| 993 | -> C | No |
| 1074 | -> C | No |
| 1141 | A -> C | No |
| 1142 | A -> G | No |
| 1235 | -> G | No |
| 1235 | -> T | No |

Variant protein HUMHPA1B_PEA_{—}1_P72 (SEQ ID NO:138) according to the present invention has an amino acid sequence as given at the end of the application; it is encoded by transcript(s) HUMHPA1B_PEA_{—}1_T16 (SEQ ID NO:39). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P72 (SEQ ID NO:138) and HPT_HUMAN (SEQ ID NO:131):

1. An isolated chimeric polypeptide encoding for HUMHPA1B_PEA_{—}1_P72 (SEQ ID NO:138), comprising a first amino acid sequence being at least 90% homologous to MSALGAVIALLLWGQLFAVDSGNDVTDIADDGCPKPPEIAHGYVEHSVRYQCKNYYK LRTEGD corresponding to amino acids 1-63 of HPT_HUMAN (SEQ ID NO:131), which also corresponds to amino acids 1-63 of HUMHPA1B_PEA_{—}1_P72 (SEQ ID NO:138), and a second amino acid sequence being at least 70%, optionally at least 80%, preferably at least 85%, more preferably at least 90% and most preferably at least 95% homologous to a polypeptide having the sequence ESGKPSAADPGWTPGCQRQLSLAG (SEQ ID NO:497) corresponding to amino acids 64-87 of HUMHPA1B_PEA_{—}1_P72 (SEQ ID NO:138), wherein said first amino acid sequence and second amino acid sequence are contiguous and in a sequential order.

2. An isolated polypeptide encoding for a tail of HUMHPA1B_PEA_{—}1_P72 (SEQ ID NO:138), comprising a polypeptide being at least 70%, optionally at least about 80%, preferably at least about 85%, more preferably at least about 90% and most preferably at least about 95% homologous to the sequence ESGKPSAADPGWTPGCQRQLSLAG (SEQ ID NO:497) in HUMHPA1B_PEA_{—}1_P72 (SEQ ID NO:138).

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P72 (SEQ ID NO:138) also has the following non-silent SNPs (Single Nucleotide Polymorphisms) as listed in Table 22, (given according to their position(s) on the amino acid sequence, with the alternative amino acid(s) listed; the last column indicates whether the SNP is known or not; the presence of known SNPs in variant protein HUMHPA1B_PEA_{—}1_P72 (SEQ ID NO:138) Sequence provides support for the deduced sequence of this variant protein according to the present invention).

TABLE 22
Amino acid mutations

| SNP position(s) on | | |
|---|---|---|
| amino acid sequence | Alternative amino acid(s) | Previously known SNP? |
| 8 | I -> | No |
| 38 | E -> D | No |
| 77 | P -> R | No |

The glycosylation sites of variant protein HUMHPA1B_PEA_{—}1_P72 (SEQ ID NO:138), as compared to the known protein Haptoglobin precursor (SEQ ID NO:131), are described in Table 23 (given according to their position(s) on the amino acid sequence in the first column; the second column indicates whether the glycosylation site is present in the variant protein; and the last column indicates whether the position is different on the variant protein).

TABLE 23
Glycosylation site(s)

| Position(s) on known amino | |
|---|---|
| acid sequence | Present in variant protein? |
| 207 | no |
| 241 | no |
| 184 | no |
| 211 | no |

Variant protein HUMHPA1B_PEA_{—}1_P72 (SEQ ID NO:138) is encoded by the following transcript(s): HUMHPA1B_PEA_{—}1_T16 (SEQ ID NO:39), for which the sequence(s) is/are given at the end of the application. The coding portion of transcript HUMHPA1B_PEA_{—}1_T16 (SEQ ID NO:39) is shown in bold; this coding portion starts at position 68 and ends at position 328. The transcript also has the following SNPs as listed in Table 24 (given according to their position on the nucleotide sequence, with the alternative nucleic acid listed; the last column indicates whether the SNP is known or not; the presence of known SNPs in variant protein HUMHPA1B_PEA_{—}1_P72 (SEQ ID NO:138) Sequence provides support for the deduced sequence of this variant protein according to the present invention).

TABLE 24
Nucleic acid SNPs

| SNP position on | | |
|---|---|---|
| nucleotide sequence | Alternative nucleic acid | Previously known SNP? |
| 40 | T -> G | No |
| 77 | C -> T | No |
| 90 | T -> | No |
| 181 | G -> T | No |
| 297 | C -> G | No |
| 362 | T -> C | Yes |
| 362 | T -> G | Yes |
| 382 | A -> | No |
| 455 | T -> C | No |
| 458 | -> G | No |
| 459 | T -> G | No |
| 497 | T -> C | No |
| 503 | G -> | No |
| 525 | T -> C | Yes |
| 530 | G -> A | No |
| 555 | A -> | No |
| 602 | T -> A | No |
| 700 | T -> C | No |
| 734 | T -> C | No |
| 754 | C -> T | No |
| 794 | -> C | No |
| 821 | T -> C | No |
| 837 | -> C | No |
| 863 | G -> | No |
| 863 | G -> C | No |
| 886 | -> C | No |
| 901 | -> C | No |
| 982 | -> C | No |
| 1049 | A -> C | No |
| 1050 | A -> G | No |
| 1143 | -> G | No |
| 1143 | -> T | No |

Variant protein HUMHPA1B_PEA_{—}1_P75 (SEQ ID NO:139) according to the present invention has an amino acid sequence as given at the end of the application; it is encoded by transcript(s) HUMHPA1B_PEA_{—}1_T19 (SEQ ID NO:40). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P75 (SEQ ID NO:139) and HPT_HUMAN (SEQ ID NO:131):

1. An isolated chimeric polypeptide encoding for HUMHPA1B_PEA_{—}1_P75 (SEQ ID NO:139), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEADDGCPKPPEIAHGYVEHSVRYQCKNY YKLRTEGDGVYTLNNEKQWINKAVGDKLPECEA corresponding to amino acids 1-147 of HPT_HUMAN (SEQ ID NO:131), which also corresponds to amino acids 1-147 of HUMHPA1B_PEA_{—}1_P75 (SEQ ID NO:139), and a second amino acid sequence being at least 90% homologous to GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 188-406 of HPT_HUMAN (SEQ ID NO:131), which also corresponds to amino acids 148-366 of HUMHPA1B_PEA_{—}1_P75 (SEQ ID NO:139), wherein said first amino acid sequence and second amino acid sequence are contiguous and in a sequential order.

2. An isolated chimeric polypeptide encoding for an edge portion of HUMHPA1B_PEA_{—}1_P75 (SEQ ID NO:139), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 147−x to 147; and ending at any of amino acid numbers 148+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMHPA1_B_PEA_{—}1_P75 (SEQ ID NO:139) also has the following non-silent SNPs (Single Nucleotide Polymorphisms) as listed in Table 25, (given according to their position(s) on the amino acid sequence, with the alternative amino acid(s) listed; the last column indicates whether the SNP is known or not; the presence of known SNPs in variant protein HUMHPA1B_PEA_{—}1_P75 (SEQ ID NO:139) Sequence provides support for the deduced sequence of this variant protein according to the present invention).

TABLE 25
Amino acid mutations

| SNP position(s) on | | |
|---|---|---|
| amino acid sequence | Alternative amino acid(s) | Previously known SNP? |
| 8 | I -> | No |
| 38 | E -> D | No |
| 79 | V -> | No |
| 116 | K -> E | No |
| 130 | E -> G | No |
| 130 | E -> K | No |
| 155 | Q -> | No |
| 181 | L -> V | No |
| 195 | K -> | No |
| 203 | S -> P | Yes |
| 213 | K -> | No |
| 261 | L -> P | No |
| 279 | P -> L | No |
| 315 | A -> | No |

The glycosylation sites of variant protein HUMHPA1B_PEA_{—}1_P75 (SEQ ID NO:139), compared to the known protein Haptoglobin precursor (SEQ ID NO:131), are described in Table 26 (given according to their position(s) on the amino acid sequence in the first column; the second column indicates whether the glycosylation site is present in the variant protein; and the last column indicates whether the position is different on the variant protein).

TABLE 26
Glycosylation site(s)

| Position(s) on | | |
|---|---|---|
| known amino | | |
| acid sequence | Present in variant protein? | Position in variant protein? |
| 207 | yes | 167 |
| 241 | yes | 201 |
| 184 | no | |
| 211 | yes | 171 |

Variant protein HUMHPA1B_PEA_{—}1_P75 (SEQ ID NO:139) is encoded by the following transcript(s): HUMHPA1B_PEA_{—}1_T19 (SEQ ID NO:40), for which the sequence(s) is/are given at the end of the application. The coding portion of transcript HUMHPA1B_PEA_{—}1_T19 (SEQ ID NO:40) is shown in bold; this coding portion starts at position 68 and ends at position 1165. The transcript also has the following SNPs as listed in Table 27 (given according to their position on the nucleotide sequence, with the alternative nucleic acid listed; the last column indicates whether the SNP is known or not; the presence of known SNPs in variant protein HUMHPA1B_PEA_{—}1_P75 (SEQ ID NO:139) Sequence provides support for the deduced sequence of this variant protein according to the present invention).

TABLE 27
Nucleic acid SNPs

| SNP position | | |
|---|---|---|
| nucleotide sequence | Alternative nucleic acid | Previously known SNP? |
| 40 | T -> G | No |
| 77 | C -> T | No |
| 90 | T -> | No |
| 181 | G -> T | No |
| 303 | T -> | No |
| 364 | A -> G | Yes |
| 413 | A -> G | No |
| 439 | G -> A | Yes |
| 455 | G -> A | No |
| 456 | A -> G | No |
| 481 | -> G | No |
| 511 | T -> C | Yes |
| 511 | T -> G | Yes |
| 531 | A -> | No |
| 604 | T -> C | No |
| 607 | -> G | No |
| 608 | T -> G | No |
| 646 | T -> C | No |
| 652 | G -> | No |
| 674 | T -> C | Yes |
| 679 | G -> A | No |
| 704 | A -> | No |
| 751 | T -> A | No |
| 849 | T -> C | No |
| 883 | T -> C | No |
| 903 | C -> T | No |
| 943 | -> C | No |
| 970 | T -> C | No |
| 986 | -> C | No |
| 1012 | G -> | No |
| 1012 | G -> C | No |
| 1035 | -> C | No |
| 1050 | -> C | No |
| 1131 | -> C | No |
| 1198 | A -> C | No |
| 1199 | A -> G | No |
| 1292 | -> G | No |
| 1292 | -> T | No |

Variant protein HUMHPA1B_PEA_{—}1_P76 (SEQ ID NO:140) according to the present invention has an amino acid sequence as given at the end of the application; it is encoded by transcript(s) HUMHPA1B_PEA_{—}1_T20 (SEQ ID NO:41). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P76 (SEQ ID NO:140) and HPT_HUMAN (SEQ ID NO:131):

1. An isolated chimeric polypeptide encoding for HUMHPA1B_PEA_{—}1_P76 (SEQ ID NO:40), comprising a first amino acid sequence being at least 90% homologous to MSALGAVIALLLWGQLFAVDSGNDVTDIADDGCPKPPEIAHGYVEHSVRYQ corresponding to amino acids 1-51 of HPT_HUMAN (SEQ ID NO:131), which also corresponds to amino acids 1-51 of HUMHPA1B_PEA_{—}1_P76 (SEQ ID NO:140), a second amino acid sequence bridging amino acid sequence comprising of L, and a third amino acid sequence being at least 90% homologous to QRILGGHLDAKGSFPWQAKMVSHHNLTTGATLINEQWLLTTAKNLFLNHSENATAKDI APTLTLYVGKKQLVEIEKVVLHPNYSQVDIGLIKLKQKVSVNERVMPICLPSKDYAEVG RVGYVSGWGRNANFKFTDHLKYVMLPVADQDQCIRHYEGSTVPEKKTPKSPVGVQPIL NEHTFCAGMSKYQEDTCYGDAGSAFAVHDLEEDTWYATGILSFDKSCAVAEYGVYVK VTSIQDWVQKTIAEN corresponding to amino acids 160-406 of HPT_HUMAN (SEQ ID NO:131), which also corresponds to amino acids 53-299 of HUMHPA1B_PEA_{—}1_P76 (SEQ ID NO:140), wherein said first amino acid sequence, second amino acid sequence and third amino acid sequence are contiguous and in a sequential order.

2. An isolated polypeptide encoding for an edge portion of HUMHPA1B_PEA_{—}1_P76 (SEQ ID NO:140), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QLQ having a structure as follows (numbering according to HUMHPA1B_PEA_{—}1_P76 (SEQ ID NO:140)): a sequence starting from any of amino acid numbers 51−x to 51; and ending at any of amino acid numbers 53+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P76 (SEQ ID NO:140) also has the following non-silent SNPs (Single Nucleotide Polymorphisms) as listed in Table 28, (given according to their position(s) on the amino acid sequence, with the alternative amino acid(s) listed; the last column indicates whether the SNP is known or not; the presence of known SNPs in variant protein HUMHPA1B_PEA_{—}1_P76 (SEQ ID NO:140) Sequence provides support for the deduced sequence of this variant protein according to the present invention).

TABLE 28
Amino acid mutations

| SNP position(s) | | |
|---|---|---|
| on amino acid | Alternative | Previously |
| sequence | amino acid(s) | known SNP? |
| 8 | I -> | No |
| 38 | E -> D | No |
| 60 | L -> V | No |
| 88 | Q -> | No |
| 114 | L -> V | No |
| 128 | K -> | No |
| 136 | S -> P | Yes |
| 146 | K -> | No |
| 194 | L -> P | No |
| 212 | P -> L | No |
| 248 | A -> | No |

The glycosylation sites of variant protein HUMHPA1B_PEA_{—}1_P76 (SEQ ID NO:140), as compared to the known protein Haptoglobin precursor (SEQ ID NO:131), are described in Table 29 (given according to their position(s) on the amino acid sequence in the first column; the second column indicates whether the glycosylation site is present in the variant protein; and the last column indicates whether the position is different on the variant protein).

TABLE 29
Glycosylation site(s)

| Position(s) on known amino | Present in | Position in |
|---|---|---|
| acid sequence | variant protein? | variant protein? |
| 207 | yes | 100 |
| 241 | yes | 134 |
| 184 | yes | 77 |
| 211 | yes | 104 |

Variant protein HUMHPA1B_PEA_{—}1_P76 (SEQ ID NO:140) is encoded by the following transcript(s): HUMHPA1B_PEA_{—}1_T20 (SEQ ID NO:41), for which the sequence(s) is/are given at the end of the application. The coding portion of transcript HUMHPA1B_PEA_{—}1_T20 (SEQ ID NO:41) is shown in bold; this coding portion starts at position 68 and ends at position 964. The transcript also has the following SNPs as listed in Table 30 (given according to their position on the nucleotide sequence, with the alternative nucleic acid listed; the last column indicates whether the SNP is known or not; the presence of known SNPs in variant protein HUMHPA1B_PEA_{—}1_P76 (SEQ ID NO:140) Sequence provides support for the deduced sequence of this variant protein according to the present invention).

TABLE 30
Nucleic acid SNPs

| SNP position on nucleotide | Alternative | Previously |
|---|---|---|
| sequence | nucleic acid | known SNP? |
| 40 | T -> G | No |
| 77 | C -> T | No |
| 90 | T -> | No |
| 181 | G -> T | No |
| 245 | C -> G | No |
| 310 | T -> C | Yes |
| 310 | T -> G | Yes |
| 330 | A -> | No |
| 403 | T -> C | No |
| 406 | -> G | No |
| 407 | T -> G | No |
| 445 | T -> C | No |
| 451 | G -> | No |
| 473 | T -> C | Yes |
| 478 | G -> A | No |
| 503 | A -> | No |
| 550 | T -> A | No |
| 648 | T -> C | No |
| 682 | T -> C | No |
| 702 | C -> T | No |
| 742 | -> C | No |
| 769 | T -> C | No |
| 785 | -> C | No |
| 811 | G -> | No |
| 811 | G -> C | No |
| 834 | -> C | No |
| 849 | -> C | No |
| 930 | -> C | No |
| 997 | A -> C | No |
| 998 | A -> G | No |
| 1091 | -> G | No |
| 1091 | -> T | No |

Variant protein HUMHPA1B_PEA_{—}1_P81 (SEQ ID NO:141) according to the present invention has an amino acid sequence as given at the end of the application; it is encoded by transcript(s) HUMHPA1B_PEA_{—}1_T27 (SEQ ID NO 42). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P81 (SEQ ID NO:141) and HPT_HUMAN (SEQ ID NO:131):

1. An isolated chimeric polypeptide encoding for HUMHPA1B_PEA_{—}1_P81 (SEQ ID NO:141), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEA corresponding to amino acids 1-88 of HPT_HUMAN (SEQ ID NO:131), which also corresponds to amino acids 1-88 of HUMHPA1B_PEA_{—}1_P81 (SEQ ID NO:141), and a second amino acid sequence being at least 90% homologous to GATLINEQWLLTTAKNLFLNHSENATAKDIAPTLTLYVGKKQLVEIEKVVLHPNYSQVD IGLIKLKQKVSVNERVMPICLPSKDYAEVGRVGYVSGWGRNANFKFTDHLKYVMLPV ADQDQCIRHYEGSTVPEKKTPKSPVGVQPILNEHTFCAGMSKYQEDTCYGDAGSAFAV HDLEEDTWYATGILSFDKSCAVAEYGVYVKVTSIQDWVQKTIAEN corresponding to amino acids 188-406 of HPT_HUMAN (SEQ ID NO:131), which also corresponds to amino acids 89-307 of HUMHPA1B_PEA_{—}1_P81 (SEQ ID NO:141), wherein said first amino acid sequence and second amino acid sequence are contiguous and in a sequential order.

2. An isolated chimeric polypeptide encoding for an edge portion of HUMHPA1B_PEA_{—}1_P81 (SEQ ID NO:141), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 88−x to 88; and ending at any of amino acid numbers 89+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P81 (SEQ ID NO:141) also has the following non-silent SNPs (Single Nucleotide Polymorphisms) as listed in Table 31, (given according to their position(s) on the amino acid sequence, with the alternative amino acid(s) listed; the last column indicates whether the SNP is known or not; the presence of known SNPs in variant protein HUMHPA1B_PEA_{—}1_P81 (SEQ ID NO:141) Sequence provides support for the deduced sequence of this variant protein according to the present invention).

TABLE 31
Amino acid mutations

| SNP position(s) on amino acid | Alternative | Previously |
|---|---|---|
| sequence | amino acid(s) | known SNP? |
| 8 | I -> | No |
| 38 | E -> D | No |
| 79 | V -> | No |
| 96 | Q -> | No |
| 122 | L -> V | No |
| 136 | K -> | No |
| 144 | S -> P | Yes |
| 154 | K -> | No |
| 202 | L -> P | No |
| 220 | P -> L | No |
| 256 | A -> | No |

The glycosylation sites of variant protein HUMHPA1B_PEA_{—}1_P81 (SEQ ID NO:141), as compared to the known protein Haptoglobin precursor (SEQ ID NO:131), are described in Table 32 (given according to their position(s) on the amino acid sequence in the first column; the second column indicates whether the glycosylation site is present in the variant protein; and the last column indicates whether the position is different on the variant protein).

TABLE 32
Glycosylation site(s)

| Position(s) on known amino | Present in | Position in |
|---|---|---|
| acid sequence | variant protein? | variant protein? |
| 207 | yes | 108 |
| 241 | yes | 142 |
| 184 | no | |
| 211 | yes | 112 |

Variant protein HUMHPA1B_PEA_{—}1_P81 (SEQ ID NO:141) is encoded by the following transcript(s): HUMHPA1B_PEA_{—}1_T27 (SEQ ID NO:42), for which the sequence(s) is/are given at the end of the application. The coding portion of transcript HUMHPA1B_PEA_{—}1_T27 (SEQ ID NO:42) is shown in bold; this coding portion starts at position 68 and ends at position 988. The transcript also has the following SNPs as listed in Table 33 (given according to their position on the nucleotide sequence, with the alternative nucleic acid listed; the last column indicates whether the SNP is known or not; the presence of known SNPs in variant protein HUMHPA1B_PEA_{—}1_P81 (SEQ ID NO:141) Sequence provides support for the deduced sequence of this variant protein according to the present invention).

TABLE 33
Nucleic acid SNPs

| SNP position on nucleotide | Alternative | Previously |
|---|---|---|
| sequence | nucleic acid | known SNP? |
| 40 | T -> G | No |
| 77 | C -> T | No |
| 90 | T -> | No |
| 181 | G -> T | No |
| 303 | T -> | No |
| 334 | T -> C | Yes |
| 334 | T -> G | Yes |
| 354 | A -> | No |
| 427 | T -> C | No |
| 430 | -> G | No |
| 431 | T -> G | No |
| 469 | T -> C | No |
| 475 | G -> | No |
| 497 | T -> C | Yes |
| 502 | G -> A | No |
| 527 | A -> | No |
| 574 | T -> A | No |
| 672 | T -> C | No |
| 706 | T -> C | No |
| 726 | C -> T | No |
| 766 | -> C | No |
| 793 | T -> C | No |
| 809 | -> C | No |
| 835 | G -> | No |
| 835 | G -> C | No |
| 858 | -> C | No |
| 873 | -> C | No |
| 954 | -> C | No |
| 1021 | A -> C | No |
| 1022 | A -> G | No |
| 1115 | -> G | No |
| 1115 | -> T | No |

Variant protein HUMHPA1B_PEA_{—}1_P83 (SEQ ID NO:142) according to the present invention has an amino acid sequence as given at the end of the application; it is encoded by transcript(s) HUMHPA1B_PEA_{—}1_T29 (SEQ ID NO:43). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P83 (SEQ ID NO:142) and HPT_HUMAN (SEQ ID NO:131):

1. An isolated chimeric polypeptide encoding for HUMHPA1B_PEA_{—}1_P83 (SEQ ID NO:142), comprising a first amino acid sequence being at least 90% homologous to MSALGAVIALLLWGQLFAVDSGNDVTDIAD corresponding to amino acids 1-30 of HPT_HUMAN (SEQ ID NO:131), which also corresponds to amino acids 1-30 of HUMHPA1B_PEA_{—}1_P83 (SEQ ID NO:142), and a second amino acid sequence being at least 70%, optionally at least 80%, preferably at least 85%, more preferably at least 90% and most preferably at least 95% homologous to a polypeptide having the sequence GFPP (SEQ ID NO:498) corresponding to amino acids 31-34 of HUMHPA1B_PEA_{—}1_P83 (SEQ ID NO:142), wherein said first amino acid sequence and second amino acid sequence are contiguous and in a sequential order.

2. An isolated polypeptide encoding for a tail of HUMHPA1B_PEA_{—}1_P83 (SEQ ID NO:142), comprising a polypeptide being at least 70%, optionally at least about 80%, preferably at least about 85%, more preferably at least about 90% and most preferably at least about 95% homologous to the sequence GFPP (SEQ ID NO:498) in HUMHPA1B_PEA_{—}1_P83 (SEQ ID NO:142).

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P83 (SEQ ID NO:142) also has the following non-silent SNPs (Single Nucleotide Polymorphisms) as listed in Table 34, (given according to their position(s) on the amino acid sequence, with the alternative amino acid(s) listed; the last column indicates whether the SNP is known or not; the presence of known SNPs in variant protein HUMHPA1B_PEA_{—}1_P83 (SEQ ID NO:142) Sequence provides support for the deduced sequence of this variant protein according to the present invention).

TABLE 34
Amino acid mutations

| SNP position(s) on amino acid | Alternative | Previously |
|---|---|---|
| sequence | amino acid(s) | known SNP? |
| 8 | I -> | No |

The glycosylation sites of variant protein HUMHPA1B_PEA_{—}1_P83 (SEQ ID NO:142), as compared to the known protein Haptoglobin precursor (SEQ ID NO:131), are described in Table 35 (given according to their position(s) on the amino acid sequence in the first column; the second column indicates whether the glycosylation site is present in the variant protein; and the last column indicates whether the position is different on the variant protein).

TABLE 35
Glycosylation site(s)

| Position(s) on known amino | Present in |
|---|---|
| acid sequence | variant protein? |
| 207 | no |
| 241 | no |
| 184 | no |
| 211 | no |

Variant protein HUMHPA1B_PEA_{—}1_P83 (SEQ ID NO:142) is encoded by the following transcript(s): HUMHPA1B_PEA_{—}1_T29 (SEQ ID NO:43), for which the sequence(s) is/are given at the end of the application. The coding portion of transcript HUMHPA1B_PEA_{—}1_T29 (SEQ ID NO:43) is shown in bold; this coding portion starts at position 68 and ends at position 169. The transcript also has the following SNPs as listed in Table 36 (given according to their position on the nucleotide sequence, with the alternative nucleic acid listed; the last column indicates whether the SNP is known or not; the presence of known SNPs in variant protein HUMHPA1B_PEA_{—}1_P83 (SEQ ID NO:142) Sequence provides support for the deduced sequence of this variant protein according to the present invention).

TABLE 36
Nucleic acid SNPs

| SNP position on | Alternative | Previously |
|---|---|---|
| nucleotide sequence | nucleic acid | known SNP? |
| 40 | T -> G | No |
| 77 | C -> T | No |
| 90 | T -> | No |
| 185 | T -> C | Yes |
| 185 | T -> G | Yes |
| 205 | A -> | No |
| 278 | T -> C | No |
| 281 | -> G | No |
| 282 | T -> G | No |
| 320 | T -> C | No |
| 326 | G -> | No |
| 348 | T -> C | Yes |
| 353 | G -> A | No |
| 378 | A -> | No |
| 425 | T -> A | No |
| 523 | T -> C | No |
| 557 | T -> C | No |
| 577 | C -> T | No |
| 617 | -> C | No |
| 644 | T -> C | No |
| 660 | -> C | No |
| 686 | G -> | No |
| 686 | G -> C | No |
| 709 | -> C | No |
| 724 | -> C | No |
| 805 | -> C | No |
| 872 | A -> C | No |
| 873 | A -> G | No |
| 966 | -> G | No |
| 966 | -> T | No |

Variant protein HUMHPA1B_PEA_{—}1_P106 (SEQ ID NO:143) according to the present invention has an amino acid sequence as given at the end of the application; it is encoded by transcript(s) HUMHPA1B_PEA_{—}1_T55 (SEQ ID NO:44. An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P106 (SEQ ID NO:143) and HPT_HUMAN_V1 (SEQ ID NO:132) (SEQ ID NO:132):

1. An isolated chimeric polypeptide encoding for HUMHPA1B_PEA_{—}1_P106 (SEQ ID NO:143), comprising a first amino acid sequence being at least 90% homologous to MSALGAVIALLLWGQLFAVDSGNDVTDIADDGCPKPPEIAHGYVEHSVRYQCKNYYK LRTEGDGVYTLNN corresponding to amino acids 1-70 of HPT_HUMAN_V1 (SEQ ID NO:132), which also corresponds to amino acids 1-70 of HUMHPA1B_PEA_{—}1_P106 (SEQ ID NO:143), a bridging amino acid E corresponding to amino acid 71 of HUMHPA1B_PEA_{—}1_P106 (SEQ ID NO:143), a bridging amino acid E corresponding to amino acid 71 of HUMHPA1B_PEA_{—}1_P106 (SEQ ID NO:143), a second amino acid sequence being at least 90% homologous to KQWINKAVGDKLPECEA corresponding to amino acids 72-88 of HPT_HUMAN_V1 (SEQ ID NO:132), which also corresponds to amino acids 72-88 of HUMHPA1B_PEA_{—}1_P106 (SEQ ID NO:143), and a third amino acid sequence being at least 70%, optionally at least 80%, preferably at least 85%, more preferably at least 90% and most preferably at least 95% homologous to a polypeptide having the sequence AHTE (SEQ ID NO:499) corresponding to amino acids 89-92 of HUMHPA1B_PEA_{—}1_P106 (SEQ ID NO:143), wherein said first amino acid sequence, bridging amino acid, bridging amino acid, second amino acid sequence and third amino acid sequence are contiguous and in a sequential order.

2. An isolated polypeptide encoding for a tail of HUMHPA1B_PEA_{—}1_P106 (SEQ ID NO:143), comprising a polypeptide being at least 70%, optionally at least about 80%, preferably at least about 85%, more preferably at least about 90% and most preferably at least about 95% homologous to the sequence AHTE (SEQ ID NO:499) in HUMHPA1B_PEA_{—}1_P106 (SEQ ID NO:143).

It should be noted that the known protein sequence (HPT_HUMAN) Has one or more changes than the sequence given at the end of the application and named as being the amino acid sequence for HPT_HUMAN_V1 (SEQ ID NO:132) (SEQ ID NO:132). These changes were previously known to occur and are listed in the table below.

TABLE 37
Changes to HPT_HUMAN_V1 (SEQ ID NO: 132)

| SNP position(s) on | |
|---|---|
| amino acid sequence | Type of change |
| 71 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P106 (SEQ ID NO:143) also has the following non-silent SNPs (Single Nucleotide Polymorphisms) as listed in Table 38, (given according to their position(s) on the amino acid sequence, with the alternative amino acid(s) listed; the last column indicates whether the SNP is known or not; the presence of known SNPs in variant protein HUMHPA1B_PEA_{—}1_P106 (SEQ ID NO:143) Sequence provides support for the deduced sequence of this variant protein according to the present invention).

TABLE 38
Amino acid mutations

| SNP position(s) on | Alternative | Previously |
|---|---|---|
| amino acid sequence | amino acid(s) | known SNP? |
| 8 | I -> | No |
| 38 | E -> D | No |
| 71 | E -> G | No |
| 71 | E -> K | No |

Variant protein HUMHPA1B_PEA_{—}1_P106 (SEQ ID NO:143) is encoded by the following transcript(s): HUMHPA1B_PEA_{—}1_T55 (SEQ ID NO:44), for which the sequence(s) is/are given at the end of the application. The coding portion of transcript HUMHPA1B_PEA_{—}1_T55 (SEQ ID NO:44) is shown in bold; this coding portion starts at position 68 and ends at position 343. The transcript also has the following SNPs as listed in Table 39 (given according to their position on the nucleotide sequence, with the alternative nucleic acid listed; the last column indicates whether the SNP is known or not; the presence of known SNPs in variant protein HUMHPA1B_PEA_{—}1_P106 (SEQ ID NO:143) Sequence provides support for the deduced sequence of this variant protein according to the present invention).

TABLE 39
Nucleic acid SNPs

| SNP position on | Alternative | Previously |
|---|---|---|
| nucleotide sequence | nucleic acid | known SNP? |
| 40 | T -> G | No |
| 77 | C -> T | No |
| 90 | T -> | No |
| 181 | G -> T | No |
| 262 | G -> A | Yes |
| 278 | G -> A | No |
| 279 | A -> G | No |
| 304 | -> G | No |
| 335 | -> C | No |
| 362 | T -> C | No |
| 378 | -> C | No |
| 404 | G -> | No |
| 404 | G -> C | No |
| 427 | -> C | No |
| 442 | -> C | No |
| 523 | -> C | No |
| 590 | A -> C | No |
| 591 | A -> G | No |
| 684 | -> G | No |
| 684 | -> T | No |

Variant protein HUMHPA1B_PEA_{—}1_P107 (SEQ ID NO:144)) according to the present invention has an amino acid sequence as given at the end of the application; it is encoded by transcript(s) HUMHPA1B_PEA_{—}1_T56 (SEQ ID NO:45). An alignment is given to the known protein (Haptoglobin precursor (SEQ ID NO:131)) at the end of the application. One or more alignments to one or more previously published protein sequences are given at the end of the application. A brief description of the relationship of the variant protein according to the present application to each such aligned protein is as follows:

Comparison report between HUMHPA1B_PEA_{—}1_P107 (SEQ ID NO:144)) and HPT_HUMAN:

1. An isolated chimeric polypeptide encoding for HUMHPA1B_PEA_{—}1_P107 (SEQ ID NO:144)), comprising a first amino acid sequence being at least 90% homologous to MSALGAVIALLLWGQLFAVDSGNDVTDI corresponding to amino acids 1-28 of HPT_HUMAN, which also corresponds to amino acids 1-28 of HUMHPA1B_PEA_{—}1_P107 (SEQ ID NO:144)), a second amino acid sequence being at least 90% homologous to ADDGCPKPPEIAHGYVEHSVRYQCKNYYKLRTEGDGVYTLNNEKQWINKAVGDKLPE CEAVCGKPKNPANPVQRILGGHLDAKGSFPWQAKMVSHHNLTT corresponding to amino acids 88-187 of HPT_HUMAN, which also corresponds to amino acids 29-128 of HUMHPA1B_PEA_{—}1_P107 (SEQ ID NO:144)), and a third amino acid sequence being at least 70%, optionally at least 80%, preferably at least 85%, more preferably at least 90% and most preferably at least 95% homologous to a polypeptide having the sequence VPLPFTTWRRTPGMRLGS (SEQ ID NO:500) corresponding to amino acids 129-146 of HUMHPA1B_PEA_{—}1_P107 (SEQ ID NO:144), wherein said first amino acid sequence, second amino acid sequence and third amino acid sequence are contiguous and in a sequential order.

2. An isolated chimeric polypeptide encoding for an edge portion of HUMHPA1B_PEA_{—}1_P107 (SEQ ID NO:144), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IA, having a structure as follows: a sequence starting from any of amino acid numbers 28−x to 28; and ending at any of amino acid numbers 29+((n−2)−x), in which x varies from 0 to n−2.

3. An isolated polypeptide encoding for a tail of HUMHPA1B_PEA_{—}1_P107 (SEQ ID NO:144), comprising a polypeptide being at least 70%, optionally at least about 80%, preferably at least about 85%, more preferably at least about 90% and most preferably at least about 95% homologous to the sequence VPLPFTTWRRTPGMRLGS (SEQ ID NO:500) in HUMHPA1B_PEA_{—}1_P107 (SEQ ID NO:144)

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P107 (SEQ ID NO:144)) also has the following non-silent SNPs (Single Nucleotide Polymorphisms) as listed in Table 40, (given according to their position(s) on the amino acid sequence, with the alternative amino acid(s) listed; the last column indicates whether the SNP is known or not; the presence of known SNPs in variant protein HUMHPA1B_PEA_{—}1_P_{—}107 (SEQ ID NO:144)) Sequence provides support for the deduced sequence of this variant protein according to the present invention).

TABLE 40
Amino acid mutations

| SNP position(s) on | Alternative | Previously |
|---|---|---|
| amino acid sequence | amino acid(s) | known SNP? |
| 8 | I -> | No |
| 38 | E -> D | No |
| 71 | E -> G | No |
| 71 | E -> K | No |
| 108 | L -> V | No |

The glycosylation sites of variant protein HUMHPA1B_PEA_{—}1_P_{—}107 (SEQ ID NO:144), as compared to the known protein Haptoglobin precursor (SEQ ID NO:131), are described in Table 41 (given according to their position(s) on the amino acid sequence in the first column; the second column indicates whether the glycosylation site is present in the variant protein; and the last column indicates whether the position is different on the variant protein).

TABLE 41
Glycosylation site(s)

| Position(s) on known | Present in | Position in |
|---|---|---|
| amino acid sequence | variant protein? | variant protein? |
| 207 | no | |
| 241 | no | |
| 184 | yes | 125 |
| 211 | no | |

Variant protein HUMHPA1B_PEA_{—}1_P107 (SEQ ID NO:144) is encoded by the following transcript(s): HUMHPA1B_PEA_{—}1_T56 (SEQ ID NO:45), for which the sequence(s) is/are given at the end of the application. The coding portion of transcript HUMHPA1B_PEA_{—}1_T56 (SEQ ID NO:45) is shown in bold; this coding portion starts at position 68 and ends at position 505. The transcript also has the following SNPs as listed in Table 42 (given according to their position on the nucleotide sequence, with the alternative nucleic acid listed; the last column indicates whether the SNP is known or not; the presence of known SNPs in variant protein HUMHPA1B_PEA_{—}1_P107 (SEQ ID NO:144) Sequence provides support for the deduced sequence of this variant protein according to the present invention).

TABLE 42
Nucleic acid SNPs

| SNP position on | Alternative | Previously |
|---|---|---|
| nucleotide sequence | nucleic acid | known SNP? |
| 40 | T -> G | No |
| 77 | C -> T | No |
| 90 | T -> | No |
| 181 | G -> T | No |
| 262 | G -> A | Yes |
| 278 | G -> A | No |
| 279 | A -> G | No |
| 304 | -> G | No |
| 337 | -> G | No |
| 389 | C -> G | No |
| 470 | -> C | No |
| 485 | -> C | No |
| 566 | -> C | No |
| 633 | A -> C | No |
| 634 | A -> G | No |
| 727 | -> G | No |
| 727 | -> T | No |

Variant protein HUMHPA1B_PEA_{—}1_P115 (SEQ ID NO:145) according to the present invention has an amino acid sequence as given at the end of the application; it is encoded by transcript(s) HUMHPA1B_PEA_{—}1_T59 (SEQ ID NO:46). An alignment is given to the known protein (Haptoglobin precursor (SEQ ID NO:131)) at the end of the application. One or more alignments to one or more previously published protein sequences are given at the end of the application. A brief description of the relationship of the variant protein according to the present invention to each such aligned protein is as follows:

Comparison report between HUMHPA1B_PEA_{—}1_P115 (SEQ ID NO:145) and HPT_HUMAN:

1. An isolated chimeric polypeptide encoding for HUMHPA1B_PEA_{—}1_P115 (SEQ ID NO:145), comprising a first amino acid sequence being at least 90% homologous to MSALGAVIALLLWGQLFAVDSGNDVTDIADDGCPKPPEIAHGYVEHSVRYQCKNYYK LRTEGDGVYTLNDKKQWINKAVGDKLPECEA corresponding to amino acids 1-88 of HPT_HUMAN, which also corresponds to amino acids 1-88 of HUMHPA1B_PEA_{—}1_P115 (SEQ ID NO:145), and a second amino acid sequence being at least 70%, optionally at least 80%, preferably at least 85%, more preferably at least 90% and most preferably at least 95% homologous to a polypeptide having the sequence GGC corresponding to amino acids 89-91 of HUMHPA1B_PEA_{—}1_P115 (SEQ ID NO:145), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because of manual inspection of known protein localization and/or gene structure.

Variant protein HUMHPA1B_PEA_{—}1_P115 (SEQ ID NO:145) also has the following non-silent SNPs (Single Nucleotide Polymorphisms) as listed in Table 43, (given according to their position(s) on the amino acid sequence, with the alternative amino acid(s) listed; the last column indicates whether the SNP is known or not; the presence of known SNPs in variant protein HUMHPA1B_PEA_{—}1_P115 (SEQ ID NO:145) Sequence provides support for the deduced sequence of this variant protein according to the present invention).

TABLE 43
Amino acid mutations

| SNP position(s) on | Alternative | Previously |
|---|---|---|
| amino acid sequence | amino acid(s) | known SNP? |
| 8 | I -> | No |
| 38 | E -> D | No |
| 79 | V -> | No |

The glycosylation sites of variant protein HUMHPA1B_PEA_{—}1_P115 (SEQ ID NO:145), as compared to the known protein Haptoglobin precursor (SEQ ID NO:131), are described in Table 44 (given according to their position(s) on the amino acid sequence in the first column; the second column indicates whether the glycosylation site is present in the variant protein; and the last column indicates whether the position is different on the variant protein).

TABLE 44
Glycosylation site(s)

| Position(s) on known | Present in |
|---|---|
| amino acid sequence | variant protein? |
| 207 | no |
| 241 | no |
| 184 | no |
| 211 | no |

Variant protein HUMHPA1B_PEA_{—}1_P115 (SEQ ID NO:145) is encoded by the following transcript(s): HUMHPA1B_PEA_{—}1_T59 (SEQ ID NO:46), for which the sequence(s) is/are given at the end of the application. The coding portion of transcript HUMHPA1B_PEA_{—}1_T59 (SEQ ID NO:46) is shown in bold; this coding portion starts at position 68 and ends at position 340. The transcript also has the following SNPs as listed in Table 45 (given according to their position on the nucleotide sequence, with the alternative nucleic acid listed; the last column indicates whether the SNP is known or not; the presence of known SNPs in variant protein HUMHPA1B_PEA_{—}1_P115 (SEQ ID NO:145) Sequence provides support for the deduced sequence of this variant protein according to the present invention).

TABLE 45
Nucleic acid SNPs

| SNP position on | Alternative | Previously |
|---|---|---|
| nucleotide sequence | nucleic acid | known SNP? |
| 40 | T -> G | No |
| 77 | C -> T | No |
| 90 | T -> | No |
| 181 | G -> T | No |
| 303 | T -> | No |
| 510 | G -> A | Yes |
| 560 | C -> T | Yes |
| 581 | C -> T | Yes |
| 615 | A -> G | Yes |

As noted above, cluster HUMHPA1B features 84 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMHPA1B_PEA_{—}_node_{—}20 (SEQ ID NO:47) according to the present invention is supported by 4 libraries. The number of libraries was determined as previously described. This segment can be found in the following transcript(s): HUMHPA1B_PEA_{—}1_T4 (SEQ ID NO:35). Table 46 below describes the starting and ending position of this segment on each transcript.

TABLE 46
Segment location on transcripts

| | Segment | Segment |
|---|---|---|
| Transcript name | starting position | ending position |
| HUMHPA1B_PEA_1_T4 (SEQ ID | 258 | 1017 |
| NO: 35) | | |

Segment cluster HUMHPA1B_PEA_{—}1_node_{—}25 (SEQ ID NO:48) according to the present invention is supported by 2 libraries. The number of libraries was determined as previously described. This segment can be found in the following transcript(s): HUMHPA1B_PEA_{—}1_T59 (SEQ ID NO:46). Table 47 below describes the starting and ending position of this segment on each transcript.

TABLE 47
Segment location on transcripts

| | Segment | Segment |
|---|---|---|
| Transcript name | starting position | ending position |
| HUMHPA1B_PEA_1_T59 | 333 | 920 |
| (SEQ ID NO: 46) | | |

Segment cluster HUMHPA1B_PEA_{—}1_node_{—}28 (SEQ ID NO:49) according to the present invention is supported by 7 libraries. The number of libraries was determined as previously described. This segment can be found in the following transcript(s): HUMHPA1B_PEA_{—}1_T6 (SEQ ID NO:36). Table 48 below describes the starting and ending position of this segment on each transcript.

TABLE 48
Segment location on transcripts

| | Segment | Segment |
|---|---|---|
| Transcript name | starting position | ending position |
| HUMHPA1B_PEA_1_T6 (SEQ ID | 435 | 1192 |
| NO: 36) | | |

Segment cluster HUMHPA1B_PEA_{—}1_node_{—}35 (SEQ ID NO:50) according to the present invention is supported by 9 libraries. The number of libraries was determined as previously described. This segment can be found in the following transcript(s): HUMHPA1B_PEA_{—}1_T7 (SEQ ID NO:37). Table 49 below describes the starting and ending position of this segment on each transcript.

TABLE 49
Segment location on transcripts

| | Segment | Segment |
|---|---|---|
| Transcript name | starting position | ending position |
| HUMHPA1B_PEA_1_T7 (SEQ ID | 524 | 1432 |
| NO: 37) | | |

Segment cluster HUMHPA1B_PEA_{—}1_node_{—}88 (SEQ ID NO:51) according to the present invention is supported by 95 libraries. The number of libraries was determined as previously described. This segment can be found in the following transcript(s): HUMHPA1B_PEA_{—}1_T1 (SEQ ID NO:34), HUMHPA1_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43, HUMHPA1B_PEA_{—}1_T55 (SEQ ID NO:44) and HUMHPA1B_PEA_{—}1_T56 (SEQ ID NO:45). Table 50 below describes the starting and ending position of this segment on each transcript.

TABLE 50
Segment location on transcripts

| | | Segment |
|---|---|---|
| | Segment | ending |
| Transcript name | starting position | position |
| HUMHPA1B_PEA_1_T1 (SEQ ID | 1155 | 1276 |
| NO: 34) | | |
| HUMHPA1B_PEA_1_T4 (SEQ ID | 2092 | 2213 |
| NO: 35) | | |
| HUMHPA1B_PEA_1_T6 (SEQ ID | 2090 | 2211 |
| NO: 36) | | |
| HUMHPA1B_PEA_1_T7 (SEQ ID | 2255 | 2376 |
| NO: 37) | | |
| HUMHPA1B_PEA_1_T12 (SEQ ID | 1155 | 1276 |
| NO: 38) | | |
| HUMHPA1B_PEA_1_T16 (SEQ ID | 1063 | 1184 |
| NO: 39) | | |
| HUMHPA1B_PEA_1_T19 (SEQ ID | 1212 | 1333 |
| NO: 40) | | |
| HUMHPA1B_PEA_1_T20 (SEQ ID | 1011 | 1132 |
| NO: 41) | | |
| HUMHPA1B_PEA_1_T27 (SEQ ID | 1035 | 1156 |
| NO: 42) | | |
| HUMHPA1B_PEA_1_T29 (SEQ ID | 886 | 1007 |
| NO: 43) | | |
| HUMHPA1B_PEA_1_T55 (SEQ ID | 604 | 725 |
| NO: 44) | | |
| HUMHPA1B_PEA_1_T56 (SEQ ID | 647 | 768 |
| NO: 45) | | |

According to an optional embodiment of the present invention, short segments related to the above cluster are also provided. These segments are up to about 120 bp in length, and so are included in a separate description.

Segment cluster HUMHPA1B_PEA_{—}1_node_{—}0 (SEQ ID NO:52) according to the present invention is supported by 45 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 51 below describes the starting and ending position of this segment on each transcript.

TABLE 51
Segment location on transcripts

| | | Segment |
|---|---|---|
| | Segment | ending |
| Transcript name | starting position | position |
| HUMHPA1B_PEA_1_T1 (SEQ ID | 1 | 63 |
| NO: 34) | | |
| HUMHPA1B_PEA_1_T4 (SEQ ID | 1 | 63 |
| NO: 35) | | |
| HUMHPA1B_PEA_1_T6 (SEQ ID | 1 | 63 |
| NO: 36) | | |
| HUMHPA1B_PEA_1_T7 (SEQ ID | 1 | 63 |
| NO: 37) | | |
| HUMHPA1B_PEA_1_T12 (SEQ ID | 1 | 63 |
| NO: 38) | | |
| HUMHPA1B_PEA_1_T16 (SEQ ID | 1 | 63 |
| NO: 39) | | |
| HUMHPA1B_PEA_1_T19 (SEQ ID | 1 | 63 |
| NO: 40) | | |
| HUMHPA1B_PEA_1_T20 (SEQ ID | 1 | 63 |
| NO: 41) | | |
| HUMHPA1B_PEA_1_T27 (SEQ ID | 1 | 63 |
| NO: 42) | | |
| HUMHPA1B_PEA_1_T29 (SEQ ID | 1 | 63 |
| NO: 43) | | |
| HUMHPA1B_PEA_1_T55 (SEQ ID | 1 | 63 |
| NO: 44) | | |
| HUMHPA1B_PEA_1_T56 (SEQ ID | 1 | 63 |
| NO: 45) | | |
| HUMHPA1B_PEA_1_T59 (SEQ ID | 1 | 63 |
| NO: 46) | | |

Segment cluster HUMHPA1B_PEA_{—}1_node_{—}1 (SEQ ID NO:53) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43, HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 52 below describes the starting and ending position of this segment on each transcript.

TABLE 52
Segment location on transcripts

| | | Segment |
|---|---|---|
| | Segment | ending |
| Transcript name | starting position | position |
| HUMHPA1B_PEA_1_T1 (SEQ ID | 64 | 72 |
| NO: 34) | | |
| HUMHPA1B_PEA_1_T4 (SEQ ID | 64 | 72 |
| NO: 35) | | |
| HUMHPA1B_PEA_1_T6 (SEQ ID | 64 | 72 |
| NO: 36) | | |
| HUMHPA1B_PEA_1_T7 (SEQ ID | 64 | 72 |
| NO: 37) | | |
| HUMHPA1B_PEA_1_T12 (SEQ ID | 64 | 72 |
| NO: 38) | | |
| HUMHPA1B_PEA_1_T16 (SEQ ID | 64 | 72 |
| NO: 39) | | |
| HUMHPA1B_PEA_1_T19 (SEQ ID | 64 | 72 |
| NO: 40) | | |
| HUMHPA1B_PEA_1_T20 (SEQ ID | 64 | 72 |
| NO: 41) | | |
| HUMHPA1B_PEA_1_T27 (SEQ ID | 64 | 72 |
| NO: 42) | | |
| HUMHPA1B_PEA_1_T29 (SEQ ID | 64 | 72 |
| NO: 43) | | |
| HUMHPA1B_PEA_1_T55 (SEQ ID | 64 | 72 |
| NO: 44) | | |
| HUMHPA1B_PEA_1_T56 (SEQ ID | 64 | 72 |
| NO: 45) | | |
| HUMHPA1B_PEA_1_T59 (SEQ ID | 64 | 72 |
| NO: 46) | | |

Segment cluster HUMHPA1B_PEA_{—}1_node_{—}3 (SEQ ID NO:54) according to the present invention can be found in the following transcript(s): HUMHPA1B_PEA I_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA I_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 53 below describes the starting and ending position of this segment on each transcript.

TABLE 53
Segment location on transcripts

| | | Segment |
|---|---|---|
| | Segment | ending |
| Transcript name | starting position | position |
| HUMHPA1B_PEA_1_T1 (SEQ ID | 73 | 97 |
| NO: 34) | | |
| HUMHPA1B_PEA_1_T4 (SEQ ID | 73 | 97 |
| NO: 35) | | |
| HUMHPA1B_PEA_1_T6 (SEQ ID | 73 | 97 |
| NO: 36) | | |
| HUMHPA1B_PEA_1_T7 (SEQ ID | 73 | 97 |
| NO: 37) | | |
| HUMHPA1B_PEA_1_T12 (SEQ ID | 73 | 97 |
| NO: 38) | | |
| HUMHPA1B_PEA_1_T16 (SEQ ID | 73 | 97 |
| NO: 39) | | |
| HUMHPA1B_PEA_1_T19 (SEQ ID | 73 | 97 |
| NO: 40) | | |
| HUMHPA1B_PEA_1_T20 (SEQ ID | 73 | 97 |
| NO: 41) | | |
| HUMHPA1B_PEA_1_T27 (SEQ ID | 73 | 97 |
| NO: 42) | | |
| HUMHPA1B_PEA_1_T29 (SEQ ID | 73 | 97 |
| NO: 43) | | |
| HUMHPA1B_PEA_1_T55 (SEQ ID | 73 | 97 |
| NO: 44) | | |
| HUMHPA1B_PEA_1_T56 (SEQ ID | 73 | 97 |
| NO: 45) | | |
| HUMHPA1B_PEA_1_T59 (SEQ ID | 73 | 97 |
| NO: 46) | | |

Segment cluster HUMHPA1B_PEA_{—}1_node_{—}4 (SEQ ID NO:55) according to the present invention can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—1}_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 54 below describes the starting and ending position of this segment on each transcript.

TABLE 54
Segment location on transcripts

| | | Segment |
|---|---|---|
| | Segment | ending |
| Transcript name | starting position | position |
| HUMHPA1B_PEA_1_T1 (SEQ ID | 98 | 112 |
| NO: 34) | | |
| HUMHPA1B_PEA_1_T4 (SEQ ID | 98 | 112 |
| NO: 35) | | |
| HUMHPA1B_PEA_1_T6 (SEQ ID | 98 | 112 |
| NO: 36) | | |
| HUMHPA1B_PEA_1_T7 (SEQ ID | 98 | 112 |
| NO: 37) | | |
| HUMHPA1B_PEA_1_T12 (SEQ ID | 98 | 112 |
| NO: 38) | | |
| HUMHPA1B_PEA_1_T16 (SEQ ID | 98 | 112 |
| NO: 39) | | |
| HUMHPA1B_PEA_1_T19 (SEQ ID | 98 | 112 |
| NO: 40) | | |
| HUMHPA1B_PEA_1_T20 (SEQ ID | 98 | 112 |
| NO: 41) | | |
| HUMHPA1B_PEA_1_T27 (SEQ ID | 98 | 112 |
| NO: 42) | | |
| HUMHPA1B_PEA_1_T29 (SEQ ID | 98 | 112 |
| NO: 43) | | |
| HUMHPA1B_PEA_1_T55 (SEQ ID | 98 | 112 |
| NO: 44) | | |
| HUMHPA1B_PEA_1_T56 (SEQ ID | 98 | 112 |
| NO: 45) | | |
| HUMHPA1B_PEA_1_T59 (SEQ ID | 98 | 112 |
| NO: 46) | | |

Segment cluster HUMHPA1B_PEA_{—}1_node_{—}5 (SEQ ID NO:56) according to the present invention is supported by 90 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43, HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 55 below describes the starting and ending position of this segment on each transcript.

TABLE 55
Segment location on transcripts

| | | Segment |
|---|---|---|
| | Segment | ending |
| Transcript name | starting position | position |
| HUMHPA1B_PEA_1_T1 (SEQ ID | 113 | 144 |
| NO: 34) | | |
| HUMHPA1B_PEA_1_T4 (SEQ ID | 113 | 144 |
| NO: 35) | | |
| HUMHPA1B_PEA_1_T6 (SEQ ID | 113 | 144 |
| NO: 36) | | |
| HUMHPA1B_PEA_1_T7 (SEQ ID | 113 | 144 |
| NO: 37) | | |
| HUMHPA1B_PEA_1_T12 (SEQ ID | 113 | 144 |
| NO: 38) | | |
| HUMHPA1B_PEA_1_T16 (SEQ ID | 113 | 144 |
| NO: 39) | | |
| HUMHPA1B_PEA_1_T19 (SEQ ID | 113 | 144 |
| NO: 40) | | |
| HUMHPA1B_PEA_1_T20 (SEQ ID | 113 | 144 |
| NO: 41) | | |
| HUMHPA1B_PEA_1_T27 (SEQ ID | 113 | 144 |
| NO: 42) | | |
| HUMHPA1B_PEA_1_T29 (SEQ ID | 113 | 144 |
| NO: 43) | | |
| HUMHPA1B_PEA_1_T55 (SEQ ID | 113 | 144 |
| NO: 44) | | |
| HUMHPA1B_PEA_1_T56 (SEQ ID | 113 | 144 |
| NO: 45) | | |
| HUMHPA1B_PEA_1_T59 (SEQ ID | 113 | 144 |
| NO: 46) | | |

Segment cluster HUMHPA1B_PEA_{—}1_node_{—}6 (SEQ ID NO:57) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44), HUMHPA1B_PEA_{—}1_T56 (SEQ ID NO:45 and HUMHPA1B_PEA_{—}1_T59 (SEQ ID NO:46). Table 56 below describes the starting and ending position of this segment on each transcript.

TABLE 56
Segment location on transcripts

| | | Segment |
|---|---|---|
| | Segment | ending |
| Transcript name | starting position | position |
| HUMHPA1B_PEA_1_T1 (SEQ ID | 145 | 150 |
| NO: 34) | | |
| HUMHPA1B_PEA_1_T4 (SEQ ID | 145 | 150 |
| NO: 35) | | |
| HUMHPA1B_PEA_1_T6 (SEQ ID | 145 | 150 |
| NO: 36) | | |
| HUMHPA1B_PEA_1_T7 (SEQ ID | 145 | 150 |
| NO: 37) | | |
| HUMHPA1B_PEA_1_T12 (SEQ ID | 145 | 150 |
| NO: 38) | | |
| HUMHPA1B_PEA_1_T16 (SEQ ID | 145 | 150 |
| NO: 39) | | |
| HUMHPA1B_PEA_1_T19 (SEQ ID | 145 | 150 |
| NO: 40) | | |
| HUMHPA1B_PEA_1_T20 (SEQ ID | 145 | 150 |
| NO: 41) | | |
| HUMHPA1B_PEA_1_T27 (SEQ ID | 145 | 150 |
| NO: 42) | | |
| HUMHPA1B_PEA_1_T29 (SEQ ID | 145 | 150 |
| NO: 43) | | |
| HUMHPA1B_PEA_1_T55 (SEQ ID | 145 | 150 |
| NO: 44) | | |
| HUMHPA1B_PEA_1_T56 (SEQ ID | 145 | 150 |
| NO: 45) | | |
| HUMHPA1B_PEA_1_T59 (SEQ ID | 145 | 150 |
| NO: 46) | | |

Segment cluster HUMHPA1B_PEA_{—}1_node_{—}7 (SEQ ID NO:58) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA 1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 57 below describes the starting and ending position of this segment on each transcript.

TABLE 57
Segment location on transcripts

| | | Segment |
|---|---|---|
| | Segment | ending |
| Transcript name | starting position | position |
| HUMHPA1B_PEA_1_T1 (SEQ ID | 151 | 155 |
| NO: 34) | | |
| HUMHPA1B_PEA_1_T4 (SEQ ID | 151 | 155 |
| NO: 35) | | |
| HUMHPA1B_PEA_1_T6 (SEQ ID | 151 | 155 |
| NO: 36) | | |
| HUMHPA1B_PEA_1_T7 (SEQ ID | 151 | 155 |
| NO: 37) | | |
| HUMHPA1B_PEA_1_T12 (SEQ ID | 151 | 155 |
| NO: 38) | | |
| HUMHPA1B_PEA_1_T16 (SEQ ID | 151 | 155 |
| NO: 39) | | |
| HUMHPA1B_PEA_1_T19 (SEQ ID | 151 | 155 |
| NO: 40) | | |
| HUMHPA1B_PEA_1_T20 (SEQ ID | 151 | 155 |
| NO: 41) | | |
| HUMHPA1B_PEA_1_T27 (SEQ ID | 151 | 155 |
| NO: 42) | | |
| HUMHPA1B_PEA_1_T29 (SEQ ID | 151 | 155 |
| NO: 43) | | |
| HUMHPA1B_PEA_1_T55 (SEQ ID | 151 | 155 |
| NO: 44) | | |
| HUMHPA1B_PEA_1_T56 (SEQ ID | 151 | 155 |
| NO: 45) | | |
| HUMHPA1B_PEA_1_T59 (SEQ ID | 151 | 155 |
| NO: 46) | | |

Segment cluster HUMHPA1B_PEA_{—}1_node_{—}10 (SEQ ID NO:59) according to the present invention is supported by 95 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46. Table 58 below describes the starting and ending position of this segment on each transcript.

TABLE 58
Segment location on transcripts

| | Segment | Segment |
|---|---|---|
| | starting | ending |
| Transcript name | position | position |
| HUMHPA1B_PEA_1_T1 (SEQ ID | 156 | 188 |
| NO: 34) | | |
| HUMHPA1B_PEA_1_T4 (SEQ ID | 156 | 188 |
| NO: 35) | | |
| HUMHPA1B_PEA_1_T6 (SEQ ID | 156 | 188 |
| NO: 36) | | |
| HUMHPA1B_PEA_1_T7 (SEQ ID | 156 | 188 |
| NO: 37) | | |
| HUMHPA1B_PEA_1_T12 (SEQ ID | 156 | 188 |
| NO: 38) | | |
| HUMHPA1B_PEA_1_T16 (SEQ ID | 156 | 188 |
| NO: 39) | | |
| HUMHPA1B_PEA_1_T19 (SEQ ID | 156 | 188 |
| NO: 40) | | |
| HUMHPA1B_PEA_1_T20 (SEQ ID | 156 | 188 |
| NO: 41) | | |
| HUMHPA1B_PEA_1_T27 (SEQ ID | 156 | 188 |
| NO: 42) | | |
| HUMHPA1B_PEA_1_T55 (SEQ ID | 156 | 188 |
| NO: 44) | | |
| HUMHPA1B_PEA_1_T56 (SEQ ID | 156 | 188 |
| NO: 45) | | |
| HUMHPA1B_PEA_1_T59 (SEQ ID | 156 | 188 |
| NO: 46) | | |

Segment cluster HUMHPA1B_PEA_{—}1_node_{—}1 (SEQ ID NO:60) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T55 (SEQ ID NO:44, HUMHPA1B_PEA_{—}1_T56 (SEQ ID NO:45) and HUMHPA1B_PEA_{—}1_T59 (SEQ ID NO:46). Table 59 below describes the starting and ending position of this segment on each transcript.

TABLE 59
Segment location on transcripts

| | Segment | Segment |
|---|---|---|
| | starting | ending |
| Transcript name | position | position |
| HUMHPA1B_PEA_1_T1 (SEQ ID | 189 | 192 |
| NO: 34) | | |
| HUMHPA1B_PEA_1_T4 (SEQ ID | 189 | 192 |
| NO: 35) | | |
| HUMHPA1B_PEA_1_T6 (SEQ ID | 189 | 192 |
| NO: 36) | | |
| HUMHPA1B_PEA_1_T7 (SEQ ID | 189 | 192 |
| NO: 37) | | |
| HUMHPA1B_PEA_1_T12 (SEQ ID | 189 | 192 |
| NO: 38) | | |
| HUMHPA1B_PEA_1_T16 (SEQ ID | 189 | 192 |
| NO: 39) | | |
| HUMHPA1B_PEA_1_T19 (SEQ ID | 189 | 192 |
| NO: 40) | | |
| HUMHPA1B_PEA_1_T20 (SEQ ID | 189 | 192 |
| NO: 41) | | |
| HUMHPA1B_PEA_1_T27 (SEQ ID | 189 | 192 |
| NO: 42) | | |
| HUMHPA1B_PEA_1_T55 (SEQ ID | 189 | 192 |
| NO: 44) | | |
| HUMHPA1B_PEA_1_T56 (SEQ ID | 189 | 192 |
| NO: 45) | | |
| HUMHPA1B_PEA_1_T59 (SEQ ID | 189 | 192 |
| NO: 46) | | |

Segment cluster HUMHPA1B_PEA_{—}1_node_{—}12 (SEQ ID NO:61) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46. Table 60 below describes the starting and ending position of this segment on each transcript.

TABLE 60
Segment location on transcripts

| | Segment | Segment |
|---|---|---|
| | starting | ending |
| Transcript name | position | position |
| HUMHPA1B_PEA_1_T1 (SEQ ID | 193 | 196 |
| NO: 34) | | |
| HUMHPA1B_PEA_1_T4 (SEQ ID | 193 | 196 |
| NO: 35) | | |
| HUMHPA1B_PEA_1_T6 (SEQ ID | 193 | 196 |
| NO: 36) | | |
| HUMHPA1B_PEA_1_T7 (SEQ ID | 193 | 196 |
| NO: 37) | | |
| HUMHPA1B_PEA_1_T12 (SEQ ID | 193 | 196 |
| NO: 38) | | |
| HUMHPA1B_PEA_1_T16 (SEQ ID | 193 | 196 |
| NO: 39) | | |
| HUMHPA1B_PEA_1_T19 (SEQ ID | 193 | 196 |
| NO: 40) | | |
| HUMHPA1B_PEA_1_T20 (SEQ ID | 193 | 196 |
| NO: 41) | | |
| HUMHPA1B_PEA_1_T27 (SEQ ID | 193 | 196 |
| NO: 42) | | |
| HUMHPA1B_PEA_1_T55 (SEQ ID | 193 | 196 |
| NO: 44) | | |
| HUMHPA1B_PEA_1_T56 (SEQ ID | 193 | 196 |
| NO: 45) | | |
| HUMHPA1B_PEA_1_T59 (SEQ ID | 193 | 196 |
| NO: 46) | | |

Segment cluster HUMHPA1B_PEA_{—}1_node_{—}13 (SEQ ID NO:62) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 61 below describes the starting and ending position of this segment on each transcript.

TABLE 61
Segment location on transcripts

| | Segment | Segment |
|---|---|---|
| | starting | ending |
| Transcript name | position | position |
| HUMHPA1B_PEA_1_T1 (SEQ ID | 197 | 217 |
| NO: 34) | | |
| HUMHPA1B_PEA_1_T4 (SEQ ID | 197 | 217 |
| NO: 35) | | |
| HUMHPA1B_PEA_1_T6 (SEQ ID | 197 | 217 |
| NO: 36) | | |
| HUMHPA1B_PEA_1_T7 (SEQ ID | 197 | 217 |
| NO: 37) | | |
| HUMHPA1B_PEA_1_T12 (SEQ ID | 197 | 217 |
| NO: 38) | | |
| HUMHPA1B_PEA_1_T16 (SEQ ID | 197 | 217 |
| NO: 39) | | |
| HUMHPA1B_PEA_1_T19 (SEQ ID | 197 | 217 |
| NO: 40) | | |
| HUMHPA1B_PEA_1_T20 (SEQ ID | 197 | 217 |
| NO: 41) | | |
| HUMHPA1B_PEA_1_T27 (SEQ ID | 197 | 217 |
| NO: 42) | | |
| HUMHPA1B_PEA_1_T55 (SEQ ID | 197 | 217 |
| NO: 44) | | |
| HUMHPA1B_PEA_1_T56 (SEQ ID | 197 | 217 |
| NO: 45) | | |
| HUMHPA1B_PEA_1_T59 (SEQ ID | 197 | 217 |
| NO: 46) | | |

Segment cluster HUMHPA1B_PEA-1_node_{—}14 (SEQ ID NO:63) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T55 (SEQ ID NO:44, HUMHPA1B_PEA_{—}1_T56 (SEQ ID NO:45) and HUMHPA1B_PEA_{—}1_T59 (SEQ ID NO:46). Table 62 below describes the starting and ending position of this segment on each transcript.

TABLE 62
Segment location on transcripts

| | Segment | Segment |
|---|---|---|
| | starting | ending |
| Transcript name | position | position |
| HUMHPA1B_PEA_1_T1 (SEQ ID | 218 | 221 |
| NO: 34) | | |
| HUMHPA1B_PEA_1_T4 (SEQ ID | 218 | 221 |
| NO: 35) | | |
| HUMHPA1B_PEA_1_T6 (SEQ ID | 218 | 221 |
| NO: 36) | | |
| HUMHPA1B_PEA_1_T7 (SEQ ID | 218 | 221 |
| NO: 37) | | |
| HUMHPA1B_PEA_1_T12 (SEQ ID | 218 | 221 |
| NO: 38) | | |
| HUMHPA1B_PEA_1_T16 (SEQ ID | 218 | 221 |
| NO: 39) | | |
| HUMHPA1B_PEA_1_T19 (SEQ ID | 218 | 221 |
| NO: 40) | | |
| HUMHPA1B_PEA_1_T20 (SEQ ID | 218 | 221 |
| NO: 41) | | |
| HUMHPA1B_PEA_1_T27 (SEQ ID | 218 | 221 |
| NO: 42) | | |
| HUMHPA1B_PEA_1_T55 (SEQ ID | 218 | 221 |
| NO: 44) | | |
| HUMHPA1B_PEA_1_T56 (SEQ ID | 218 | 221 |
| NO: 45) | | |
| HUMHPA1B_PEA_1_T59 (SEQ ID | 218 | 221 |
| NO: 46) | | |

Segment cluster HUMHPA1B_PEA_{—}1 node_{—}15 (SEQ ID NO:64) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 63 below describes the starting and ending position of this segment on each transcript.

TABLE 63
Segment location on transcripts

| | Segment | Segment |
|---|---|---|
| | starting | ending |
| Transcript name | position | position |
| HUMHPA1B_PEA_1_T1 (SEQ ID | 222 | 231 |
| NO: 34) | | |
| HUMHPA1B_PEA_1_T4 (SEQ ID | 222 | 231 |
| NO: 35) | | |
| HUMHPA1B_PEA_1_T6 (SEQ ID | 222 | 231 |
| NO: 36) | | |
| HUMHPA1B_PEA_1_T7 (SEQ ID | 222 | 231 |
| NO: 37) | | |
| HUMHPA1B_PEA_1_T12 (SEQ ID | 222 | 231 |
| NO: 38) | | |
| HUMHPA1B_PEA_1_T16 (SEQ ID | 222 | 231 |
| NO: 39) | | |
| HUMHPA1B_PEA_1_T19 (SEQ ID | 222 | 231 |
| NO: 40) | | |
| HUMHPA1B_PEA_1_T27 (SEQ ID | 222 | 231 |
| NO: 42) | | |
| HUMHPA1B_PEA_1_T55 (SEQ ID | 222 | 231 |
| NO: 44) | | |
| HUMHPA1B_PEA_1_T56 (SEQ ID | 222 | 231 |
| NO: 45) | | |
| HUMHPA1B_PEA_1_T59 (SEQ ID | 222 | 231 |
| NO: 46) | | |

Segment cluster HUMHPA1B_PEA_{—}1_node_{—}16 (SEQ ID NO:65) according to the present invention can be found in the following transcript(s): HUMHPA1B_PEA_{—1}_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 64 below describes the starting and ending position of this segment on each transcript.

TABLE 64
Segment location on transcripts

| | Segment | Segment |
|---|---|---|
| | starting | ending |
| Transcript name | position | position |
| HUMHPA1B_PEA_1_T1 (SEQ ID | 232 | 238 |
| NO: 34) | | |
| HUMHPA1B_PEA_1_T4 (SEQ ID | 232 | 238 |
| NO: 35) | | |
| HUMHPA1B_PEA_1_T6 (SEQ ID | 232 | 238 |
| NO: 36) | | |
| HUMHPA1B_PEA_1_T7 (SEQ ID | 232 | 238 |
| NO: 37) | | |
| HUMHPA1B_PEA_1_T12 (SEQ ID | 232 | 238 |
| NO: 38) | | |
| HUMHPA1B_PEA_1_T16 (SEQ ID | 232 | 238 |
| NO: 39) | | |
| HUMHPA1B_PEA_1_T19 (SEQ ID | 232 | 238 |
| NO: 40) | | |
| HUMHPA1B_PEA_1_T27 (SEQ ID | 232 | 238 |
| NO: 42) | | |
| HUMHPA1B_PEA_1_T55 (SEQ ID | 232 | 238 |
| NO: 44) | | |
| HUMHPA1B_PEA_1_T56 (SEQ ID | 232 | 238 |
| NO: 45) | | |
| HUMHPA1B_PEA_1_T59 (SEQ ID | 232 | 238 |
| NO: 46) | | |

Segment cluster HUMHPA1B_PEA_{—}1_node_{—}17 (SEQ ID NO:66) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 65 below describes the starting and ending position of this segment on each transcript.

TABLE 65
Segment location on transcripts

| | Segment | Segment |
|---|---|---|
| | starting | ending |
| Transcript name | position | position |
| HUMHPA1B_PEA_1_T1 (SEQ ID | 239 | 243 |
| NO: 34) | | |
| HUMHPA1B_PEA_1_T4 (SEQ ID | 239 | 243 |
| NO: 35) | | |
| HUMHPA1B_PEA_1_T6 (SEQ ID | 239 | 243 |
| NO: 36) | | |
| HUMHPA1B_PEA_1_T7 (SEQ ID | 239 | 243 |
| NO: 37) | | |
| HUMHPA1B_PEA_1_T12 (SEQ ID | 239 | 243 |
| NO: 38) | | |
| HUMHPA1B_PEA_1_T16 (SEQ ID | 239 | 243 |
| NO: 39) | | |
| HUMHPA1B_PEA_1_T19 (SEQ ID | 239 | 243 |
| NO: 40) | | |
| HUMHPA1B_PEA_1_T27 (SEQ ID | 239 | 243 |
| NO: 42) | | |
| HUMHPA1B_PEA_1_T55 (SEQ ID | 239 | 243 |
| NO: 44) | | |
| HUMHPA1B_PEA_1_T56 (SEQ ID | 239 | 243 |
| NO: 45) | | |
| HUMHPA1B_PEA_1_T59 (SEQ ID | 239 | 243 |
| NO: 46) | | |

Segment cluster HUMHPA1B_PEA-1_node_{—}18 (SEQ ID NO:67) according to the present invention can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T27 (SEQ ID NO:42, HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 66 below describes the starting and ending position of this segment on each transcript.

TABLE 66
Segment location on transcripts

| | Segment | Segment |
|---|---|---|
| | starting | ending |
| Transcript name | position | position |
| HUMHPA1B_PEA_1_T1 (SEQ ID | 244 | 247 |
| NO: 34) | | |
| HUMHPA1B_PEA_1_T4 (SEQ ID | 244 | 247 |
| NO: 35) | | |
| HUMHPA1B_PEA_1_T6 (SEQ ID | 244 | 247 |
| NO: 36) | | |
| HUMHPA1B_PEA_1_T7 (SEQ ID | 244 | 247 |
| NO: 37) | | |
| HUMHPA1B_PEA_1_T12 (SEQ ID | 244 | 247 |
| NO: 38) | | |
| HUMHPA1B_PEA_1_T16 (SEQ ID | 244 | 247 |
| NO: 39) | | |
| HUMHPA1B_PEA_1_T19 (SEQ ID | 244 | 247 |
| NO: 40) | | |
| HUMHPA1B_PEA_1_T27 (SEQ ID | 244 | 247 |
| NO: 42) | | |
| HUMHPA1B_PEA_1_T55 (SEQ ID | 244 | 247 |
| NO: 44) | | |
| HUMHPA1B_PEA_1_T56 (SEQ ID | 244 | 247 |
| NO: 45) | | |
| HUMHPA1B_PEA_1_T59 (SEQ ID | 244 | 247 |
| NO: 46) | | |

Segment cluster HUMHPA1B_PEA_{—}1_node_{—}19 (SEQ ID NO:68) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T27 (SEQ ID NO:42), HUMHPA1B_PEA_{—}1_T55 (SEQ ID NO:44), HUMHPA1B_PEA_{—}1_T56 (SEQ ID NO:45) and HUMHPA1B_PEA_{—}1_T59 (SEQ ID NO:46). Table 67 below describes the starting and ending position of this segment on each transcript.

TABLE 67
Segment location on transcripts

| | Segment | Segment |
|---|---|---|
| | starting | ending |
| Transcript name | position | position |
| HUMHPA1B_PEA_1_T1 (SEQ ID | 248 | 257 |
| NO: 34) | | |
| HUMHPA1B_PEA_1_T4 (SEQ ID | 248 | 257 |
| NO: 35) | | |
| HUMHPA1B_PEA_1_T6 (SEQ ID | 248 | 257 |
| NO: 36) | | |
| HUMHPA1B_PEA_1_T7 (SEQ ID | 248 | 257 |
| NO: 37) | | |
| HUMHPA1B_PEA_1_T12 (SEQ ID | 248 | 257 |
| NO: 38) | | |
| HUMHPA1B_PEA_1_T16 (SEQ ID | 248 | 257 |
| NO: 39) | | |
| HUMHPA1B_PEA_1_T19 (SEQ ID | 248 | 257 |
| NO: 40) | | |
| HUMHPA1B_PEA_1_T27 (SEQ ID | 248 | 257 |
| NO: 42) | | |
| HUMHPA1B_PEA_1_T55 (SEQ ID | 248 | 257 |
| NO: 44) | | |
| HUMHPA1B_PEA_1_T56 (SEQ ID | 248 | 257 |
| NO: 45) | | |
| HUMHPA1B_PEA_1_T59 (SEQ ID | 248 | 257 |
| NO: 46) | | |

Segment cluster HUMHPA1B_PEA_{—}1_node_{—}21 (SEQ ID NO:69) according to the present invention is supported by 66 libraries. The number of libraries was determined as previously described. This segment can be found in the following transcript(s): HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T19 (SEQ ID NO:40), HUMHPA1B_PEA_{—}1_T27 (SEQ ID NO:42) and HUMHPA1B_PEA_{—}1_T59 (SEQ ID NO:46). Table 68 below describes the starting and ending position of this segment on each transcript.

TABLE 68
Segment location on transcripts

| | | Segment |
|---|---|---|
| | Segment | ending |
| Transcript name | starting position | position |
| HUMHPA1B_PEA_1_T4 (SEQ ID | 1018 | 1043 |
| NO: 35) | | |
| HUMHPA1B_PEA_1_T6 (SEQ ID | 258 | 283 |
| NO: 36) | | |
| HUMHPA1B_PEA_1_T7 (SEQ ID | 258 | 283 |
| NO: 37) | | |
| HUMHPA1B_PEA_1_T12 (SEQ ID | 258 | 283 |
| NO: 38) | | |
| HUMHPA1B_PEA_1_T19 (SEQ ID | 258 | 283 |
| NO: 40) | | |
| HUMHPA1B_PEA_1_T27 (SEQ ID | 258 | 283 |
| NO: 42) | | |
| HUMHPA1B_PEA_1_T59 (SEQ ID | 258 | 283 |
| NO: 46) | | |

Segment cluster HUMHPA1B_PEA_{—}1_node_{—}22 (SEQ ID NO:70) according to the present invention can be found in the following transcript(s): HUMHPA1B_PEA_{—}1_T4 (SEQ ID NO:35), HUMHPA1B_PEA_{—}1_T6 (SEQ ID NO:36), HUMHPA1B_PEA_{—}_T7 (SEQ ID NO:37), HUMHPA1B_PEA_{—}1_T12 (SEQ ID NO:38), HUMHPA1B_PEA_{—}1_T19 (SEQ ID NO:40), HUMHPA1B_PEA_{—}1_T27 (SEQ ID NO:42) and HUMHPA1B_PEA_{—}1_T59 (SEQ ID NO:46). Table 69 below describes the starting and ending position of this segment on each transcript.

TABLE 69
Segment location on transcripts

| | | Segment |
|---|---|---|
| | Segment | ending |
| Transcript name | starting position | position |
| HUMHPA1B_PEA_1_T4 (SEQ ID | 1044 | 1059 |
| NO: 35) | | |
| HUMHPA1B_PEA_1_T6 (SEQ ID | 284 | 299 |
| NO: 36) | | |
| HUMHPA1B_PEA_1_T7 (SEQ ID | 284 | 299 |
| NO: 37) | | |
| HUMHPA1B_PEA_1_T12 (SEQ ID | 284 | 299 |
| NO: 38) | | |
| HUMHPA1B_PEA_1_T19 (SEQ ID | 284 | 299 |
| NO: 40) | | |
| HUMHPA1B_PEA_1_T27 (SEQ ID | 284 | 299 |
| NO: 42) | | |
| HUMHPA1B_PEA_1_T59 (SEQ ID | 284 | 299 |
| NO: 46) | | |

Segment cluster HUMHPA1B_PEA_{—}1_node_{—}23 (SEQ ID NO:71) according to the present invention can be found in the following transcript(s): HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38, HUMHPA1B_PEA_{—}1_T19 (SEQ ID NO:40), HUMHPA1B_PEA_{—}1_T27 (SEQ ID NO:42) and HUMHPA1B_PEA_{—}1_T59 (SEQ ID NO:46). Table 70 below describes the starting and ending position of this segment on each transcript.

TABLE 70
Segment location on transcripts

| | | Segment |
|---|---|---|
| | Segment | ending |
| Transcript name | starting position | position |
| HUMHPA1B_PEA_1_T4 (SEQ ID | 1060 | 1077 |
| NO: 35) | | |
| HUMHPA1B_PEA_1_T6 (SEQ ID | 300 | 317 |
| NO: 36) | | |
| HUMHPA1B_PEA_1_T7 (SEQ ID | 300 | 317 |
| NO: 37) | | |
| HUMHPA1B_PEA_1_T12 (SEQ ID | 300 | 317 |
| NO: 38) | | |
| HUMHPA1B_PEA_1_T19 (SEQ ID | 300 | 317 |
| NO: 40) | | |
| HUMHPA1B_PEA_1_T27 (SEQ ID | 300 | 317 |
| NO: 42) | | |
| HUMHPA1B_PEA_1_T59 (SEQ ID | 300 | 317 |
| NO: 46) | | |

Segment cluster HUMHPA1B_PEA_{—}1_node_{—}24 (SEQ ID NO:72) according to the present invention can be found in the following transcript(s): HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T19 (SEQ ID NO:40), HUMHPA1B_PEA_{—}1_T27 (SEQ ID NO:42) and HUMHPA1B_PEA_{—}1_T59 (SEQ ID NO:46). Table 71 below describes the starting and ending position of this segment on each transcript.

TABLE 71
Segment location on transcripts

| | | Segment |
|---|---|---|
| | Segment | ending |
| Transcript name | starting position | position |
| HUMHPA1B_PEA_1_T4 (SEQ ID | 1078 | 1092 |
| NO: 35) | | |
| HUMHPA1B_PEA_1_T6 (SEQ ID | 318 | 332 |
| NO: 36) | | |
| HUMHPA1B_PEA_1_T7 (SEQ ID | 318 | 332 |
| NO: 37) | | |
| HUMHPA1B_PEA_1_T12 (SEQ ID | 318 | 332 |
| NO: 38) | | |
| HUMHPA1B_PEA_1_T19 (SEQ ID | 318 | 332 |
| NO: 40) | | |
| HUMHPA1B_PEA_1_T27 (SEQ ID | 318 | 332 |
| NO: 42) | | |
| HUMHPA1B_PEA_1_T59 (SEQ ID | 318 | 332 |
| NO: 46) | | |

Segment cluster HUMHPA1B_PEA_{—}1_node_{—}27 (SEQ ID NO:73) according to the present invention is supported by 62 libraries. The number of libraries was determined as previously described. This segment can be found in the following transcript(s): HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37) and HUMHPA1B_PEA_{—}1_T19 (SEQ ID NO:40). Table 72 below describes the starting and ending position of this segment on each transcript.

TABLE 72
Segment location on transcripts

| | | Segment |
|---|---|---|
| | Segment | ending |
| Transcript name | starting position | position |
| HUMHPA1B_PEA_1_T4 (SEQ ID | 1093 | 1194 |
| NO: 35) | | |
| HUMHPA1B_PEA_1_T6 (SEQ ID | 333 | 434 |
| NO: 36) | | |
| HUMHPA1B_PEA_1_T7 (SEQ ID | 333 | 434 |
| NO: 37) | | |
| HUMHPA1B_PEA_1_T19 (SEQ ID | 333 | 434 |
| NO: 40) | | |

Segment cluster HUMHPA1B_PEA_{—}1_node_{—}29 (SEQ ID NO:74) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T19 (SEQ ID NO:40), HUMHPA1B_PEA_{—}1_T55 (SEQ ID NO:44) and HUMHPA1B_PEA_{—}1_T56 (SEQ ID NO:45). Table 73 below describes the starting and ending position of this segment on each transcript.

TABLE 73
Segment location on transcripts

| | | Segment |
|---|---|---|
| | Segment | ending |
| Transcript name | starting position | position |
| HUMHPA1B_PEA_1_T1 (SEQ ID | 258 | 277 |
| NO: 34) | | |
| HUMHPA1B_PEA_1_T4 (SEQ ID | 1195 | 1214 |
| NO: 35) | | |
| HUMHPA1B_PEA_1_T6 (SEQ ID | 1193 | 1212 |
| NO: 36) | | |
| HUMHPA1B_PEA_1_T7 (SEQ ID | 435 | 454 |
| NO: 37) | | |
| HUMHPA1B_PEA_1_T19 (SEQ ID | 435 | 454 |
| NO: 40) | | |
| HUMHPA1B_PEA_1_T55 (SEQ ID | 258 | 277 |
| NO: 44) | | |
| HUMHPA1B_PEA_1_T56 (SEQ ID | 258 | 277 |
| NO: 45) | | |

Segment cluster HUMHPA1B_PEA_{—}1_node_{—}30 (SEQ ID NO:75) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T19 (SEQ ID NO:40), HUMHPA1B_PEA_{—}1_T55 (SEQ ID NO:44) and HUMHPA1B_PEA_{—}1_T56 (SEQ ID NO:45). Table 74 below describes the starting and ending position of this segment on each transcript.

TABLE 74
Segment location on transcripts

| | | Segment |
|---|---|---|
| | Segment | ending |
| Transcript name | starting position | position |
| HUMHPA1B_PEA_1_T1 (SEQ ID | 278 | 283 |
| NO: 34) | | |
| HUMHPA1B_PEA_1_T4 (SEQ ID | 1215 | 1220 |
| NO: 35) | | |
| HUMHPA1B_PEA_1_T6 (SEQ ID | 1213 | 1218 |
| NO: 36) | | |
| HUMHPA1B_PEA_1_T7 (SEQ ID | 455 | 460 |
| NO: 37) | | |
| HUMHPA1B_PEA_1_T19 (SEQ ID | 455 | 460 |
| NO: 40) | | |
| HUMHPA1B_PEA_1_T55 (SEQ ID | 278 | 283 |
| NO: 44) | | |
| HUMHPA1B_PEA_1_T56 (SEQ ID | 278 | 283 |
| NO: 45) | | |

Segment cluster HUMHPA1B_PEA_{—}1_node_{—}31 (SEQ ID NO:76) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T19 (SEQ ID NO:40), HUMHPA1B_PEA_{—}1_T55 (SEQ ID NO:44) and HUMHPA1B_PEA_{—}1_T56 (SEQ ID NO:45). Table 75 below describes the starting and ending position of this segment on each transcript.

TABLE 75
Segment location on transcripts

| | | Segment |
|---|---|---|
| | Segment | ending |
| Transcript name | starting position | position |
| HUMHPA1B_PEA_1_T1 (SEQ ID | 284 | 289 |
| NO: 34) | | |
| HUMHPA1B_PEA_1_T4 (SEQ ID | 1221 | 1226 |
| NO: 35) | | |
| HUMHPA1B_PEA_1_T6 (SEQ ID | 1219 | 1224 |
| NO: 36) | | |
| HUMHPA1B_PEA_1_T7 (SEQ ID | 461 | 466 |
| NO: 37) | | |
| HUMHPA1B_PEA_1_T19 (SEQ ID | 461 | 466 |
| NO: 40) | | |
| HUMHPA1B_PEA_1_T55 (SEQ ID | 284 | 289 |
| NO: 44) | | |
| HUMHPA1B_PEA_1_T56 (SEQ ID | 284 | 289 |
| NO: 45) | | |

Segment cluster HUMHPA1B_PEA_{—}1_node_{—}32 (SEQ ID NO:77) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T19 (SEQ ID NO:40), HUMHPA1B_PEA_{—}1_T55 (SEQ ID NO:44) and HUMHPA1B_PEA_{—}1_T56 (SEQ ID NO:45). Table 76 below describes the starting and ending position of this segment on each transcript.

TABLE 76
Segment location on transcripts

| | | Segment |
|---|---|---|
| | Segment | ending |
| Transcript name | starting position | position |
| HUMHPA1B_PEA_1_T1 (SEQ ID | 290 | 299 |
| NO: 34) | | |
| HUMHPA1B_PEA_1_T4 (SEQ ID | 1227 | 1236 |
| NO: 35) | | |
| HUMHPA1B_PEA_1_T6 (SEQ ID | 1225 | 1234 |
| NO: 36) | | |
| HUMHPA1B_PEA_1_T7 (SEQ ID | 467 | 476 |
| NO: 37) | | |
| HUMHPA1B_PEA_1_T19 (SEQ ID | 467 | 476 |
| NO: 40) | | |
| HUMHPA1B_PEA_1_T55 (SEQ ID | 290 | 299 |
| NO: 44) | | |
| HUMHPA1B_PEA_1_T56 (SEQ ID | 290 | 299 |
| NO: 45) | | |

Segment cluster HUMHPA1B_PEA_{—}1_node_{—}33 (SEQ ID NO:78) according to the present invention is supported by 88 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B PEA_{—}1_T7 (SEQ ID NO:37), HUMHPA1B_PEA_{—}1_T19 (SEQ ID NO:40), HUMHPA1B_PEA_{—}1_T55 (SEQ ID NO:44) and HUMHPA1B_PEA_{—}1_T56 (SEQ ID NO:45). Table 77 below describes the starting and ending position of this segment on each transcript.

TABLE 77
Segment location on transcripts

| | | Segment |
|---|---|---|
| | Segment | ending |
| Transcript name | starting position | position |
| HUMHPA1B_PEA_1_T1 (SEQ ID | 300 | 332 |
| NO: 34) | | |
| HUMHPA1B_PEA_1_T4 (SEQ ID | 1237 | 1269 |
| NO: 35) | | |
| HUMHPA1B_PEA_1_T6 (SEQ ID | 1235 | 1267 |
| NO: 36) | | |
| HUMHPA1B_PEA_1_T7 (SEQ ID | 477 | 509 |
| NO: 37) | | |
| HUMHPA1B_PEA_1_T19 (SEQ ID | 477 | 509 |
| NO: 40) | | |
| HUMHPA1B_PEA_1_T55 (SEQ ID | 300 | 332 |
| NO: 44) | | |
| HUMHPA1B_PEA_1_T56 (SEQ ID | 300 | 332 |
| NO: 45) | | |

Segment cluster HUMHPA1B_PEA_{—}1_node_{—}34 (SEQ ID NO:79) according to the present invention can be found in the following transcript(s): HUMHPA1B_PEA_{—}1_T7 (SEQ ID NO:37). Table 78 below describes the starting and ending position of this segment on each transcript.

TABLE 78
Segment location on transcripts

| | Segment | Segment |
|---|---|---|
| Transcript name | starting position | ending position |
| HUMHPA1B_PEA_1_T7 (SEQ ID | 510 | 523 |
| NO: 37) | | |

Segment cluster HUMHPA1B_PEA_{—}1_node_{—}36 (SEQ ID NO:80) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38) and HUMHPA1B_PEA_{—}1_T56 (SEQ ID NO:45). Table 79 below describes the starting and ending position of this segment on each transcript.

TABLE 79
Segment location on transcripts

| | Segment | |
|---|---|---|
| | starting | Segment |
| Transcript name | position | ending position |
| HUMHPA1B_PEA_1_T1 (SEQ ID | 333 | 343 |
| NO: 34) | | |
| HUMHPA1B_PEA_1_T4 (SEQ ID | 1270 | 1280 |
| NO: 35) | | |
| HUMHPA1B_PEA_1_T6 (SEQ ID | 1268 | 1278 |
| NO: 36) | | |
| HUMHPA1B_PEA_1_T7 (SEQ ID | 1433 | 1443 |
| NO: 37) | | |
| HUMHPA1B_PEA_1_T12 (SEQ ID | 333 | 343 |
| NO: 38) | | |
| HUMHPA1B_PEA_1_T56 (SEQ ID | 333 | 343 |
| NO: 45) | | |

Segment cluster HUMHPA1B_PEA_{—}1_node_{—}37 (SEQ ID NO:81) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38) and HUMHPA1B_PEA_{—}1_T56 (SEQ ID NO:45). Table 80 below describes the starting and ending position of this segment on each transcript.

TABLE 80
Segment location on transcripts

| | Segment | |
|---|---|---|
| | starting | Segment |
| Transcript name | position | ending position |
| HUMHPA1B_PEA_1_T1 (SEQ ID | 344 | 349 |
| NO: 34) | | |
| HUMHPA1B_PEA_1_T4 (SEQ ID | 1281 | 1286 |
| NO: 35) | | |
| HUMHPA1B_PEA_1_T6 (SEQ ID | 1279 | 1284 |
| NO: 36) | | |
| HUMHPA1B_PEA_1_T7 (SEQ ID | 1444 | 1449 |
| NO: 37) | | |
| HUMHPA1B_PEA_1_T12 (SEQ ID | 344 | 349 |
| NO: 38) | | |
| HUMHPA1B_PEA_1_T56 (SEQ ID | 344 | 349 |
| NO: 45) | | |

Segment cluster HUMHPA1B_PEA_{—}1_node_{—}38 (SEQ ID NO:82) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39) and HUMHPA1B_PEA_{—}1_T56 (SEQ ID NO:45). Table 81 below describes the starting and ending position of this segment on each transcript.

TABLE 81
Segment location on transcripts

| | Segment | |
|---|---|---|
| | starting | Segment |
| Transcript name | position | ending position |
| HUMHPA1B_PEA_1_T1 (SEQ ID | 350 | 361 |
| NO: 34) | | |
| HUMHPA1B_PEA_1_T4 (SEQ ID | 1287 | 1298 |
| NO: 35) | | |
| HUMHPA1B_PEA_1_T6 (SEQ ID | 1285 | 1296 |
| NO: 36) | | |
| HUMHPA1B_PEA_1_T7 (SEQ ID | 1450 | 1461 |
| NO: 37) | | |
| HUMHPA1B_PEA_1_T12 (SEQ ID | 350 | 361 |
| NO: 38) | | |
| HUMHPA1B_PEA_1_T16 (SEQ ID | 258 | 269 |
| NO: 39) | | |
| HUMHPA1B_PEA_1_T56 (SEQ ID | 350 | 361 |
| NO: 45) | | |

Segment cluster HUMHPA1B_PEA_{—}1_node_{—}39 (SEQ ID NO:83) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39) and HUMHPA1B_PEA_{—}1_T56 (SEQ ID NO:45). Table 82 below describes the starting and ending position of this segment on each transcript.

TABLE 82
Segment location on transcripts

| | Segment | |
|---|---|---|
| | starting | Segment |
| Transcript name | position | ending position |
| HUMHPA1B_PEA_1_T1 (SEQ ID | 362 | 365 |
| NO: 34) | | |
| HUMHPA1B_PEA_1_T4 (SEQ ID | 1299 | 1302 |
| NO: 35) | | |
| HUMHPA1B_PEA_1_T6 (SEQ ID | 1297 | 1300 |
| NO: 36) | | |
| HUMHPA1B_PEA_1_T7 (SEQ ID | 1462 | 1465 |
| NO: 37) | | |
| HUMHPA1B_PEA_1_T12 (SEQ ID | 362 | 365 |
| NO: 38) | | |
| HUMHPA1B_PEA_1_T16 (SEQ ID | 270 | 273 |
| NO: 39) | | |
| HUMHPA1B_PEA_1_T56 (SEQ ID | 362 | 365 |
| NO: 45) | | |

Segment cluster HUMHPA1B_PEA_{—}1_node_{—}40 (SEQ ID NO:84) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T20 (SEQ ID NO:41) and HUMHPA1B_PEA_{—}1_T56 (SEQ ID NO:45). Table 83 below describes the starting and ending position of this segment on each transcript.

TABLE 83
Segment location on transcripts

| | Segment | |
|---|---|---|
| | starting | Segment |
| Transcript name | position | ending position |
| HUMHPA1B_PEA_1_T1 (SEQ ID | 366 | 370 |
| NO: 34) | | |
| HUMHPA1B_PEA_1_T4 (SEQ ID | 1303 | 1307 |
| NO: 35) | | |
| HUMHPA1B_PEA_1_T6 (SEQ ID | 1301 | 1305 |
| NO: 36) | | |
| HUMHPA1B_PEA_1_T7 (SEQ ID | 1466 | 1470 |
| NO: 37) | | |
| HUMHPA1B_PEA_1_T12 (SEQ ID | 366 | 370 |
| NO: 38) | | |
| HUMHPA1B_PEA_1_T16 (SEQ ID | 274 | 278 |
| NO: 39) | | |
| HUMHPA1B_PEA_1_T20 (SEQ ID | 222 | 226 |
| NO: 41) | | |
| HUMHPA1B_PEA_1_T56 (SEQ ID | 366 | 370 |
| NO: 45) | | |

Segment cluster HUMHPA1B_PEA_{—}1_node_{—}41 (SEQ ID NO:85) according to the present invention can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T20 (SEQ ID NO:41) and HUMHPA1B_PEA_{—}1_T56 (SEQ ID NO:45). Table 84 below describes the starting and ending position of this segment on each transcript.

TABLE 84
Segment location on transcripts

| | Segment | |
|---|---|---|
| | starting | Segment |
| Transcript name | position | ending position |
| HUMHPA1B_PEA_1_T1 (SEQ ID | 371 | 376 |
| NO: 34) | | |
| HUMHPA1B_PEA_1_T4 (SEQ ID | 1308 | 1313 |
| NO: 35) | | |
| HUMHPA1B_PEA_1_T6 (SEQ ID | 1306 | 1311 |
| NO: 36) | | |
| HUMHPA1B_PEA_1_T7 (SEQ ID | 1471 | 1476 |
| NO: 37) | | |
| HUMHPA1B_PEA_1_T12 (SEQ ID | 371 | 376 |
| NO: 38) | | |
| HUMHPA1B_PEA_1_T16 (SEQ ID | 279 | 284 |
| NO: 39) | | |
| HUMHPA1B_PEA_1_T20 (SEQ ID | 227 | 232 |
| NO: 41) | | |
| HUMHPA1B_PEA_1_T56 (SEQ ID | 371 | 376 |
| NO: 45) | | |

Segment cluster HUMHPA1B_PEA_{—}1_node_{—}42 (SEQ ID NO:86) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T20 (SEQ ID NO:41) and HUMHPA1B_PEA_{—}1_T56 (SEQ ID NO:45). Table 85 below describes the starting and ending position of this segment on each transcript.

TABLE 85
Segment location on transcripts

| | Segment | |
|---|---|---|
| | starting | Segment |
| Transcript name | position | ending position |
| HUMHPA1B_PEA_1_T1 (SEQ ID | 377 | 388 |
| NO: 34) | | |
| HUMHPA1B_PEA_1_T4 (SEQ ID | 1314 | 1325 |
| NO: 35) | | |
| HUMHPA1B_PEA_1_T6 (SEQ ID | 1312 | 1323 |
| NO: 36) | | |
| HUMHPA1B_PEA_1_T7 (SEQ ID | 1477 | 1488 |
| NO: 37) | | |
| HUMHPA1B_PEA_1_T12 (SEQ ID | 377 | 388 |
| NO: 38) | | |
| HUMHPA1B_PEA_1_T16 (SEQ ID | 285 | 296 |
| NO: 39) | | |
| HUMHPA1B_PEA_1_T20 (SEQ ID | 233 | 244 |
| NO: 41) | | |
| HUMHPA1B_PEA_1_T56 (SEQ ID | 377 | 388 |
| NO: 45) | | |

Segment cluster HUMHPA1B_PEA_{—}1_node_{—}43 (SEQ ID NO:87) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T20 (SEQ ID NO:41) and HUMHPA1B_PEA_{—}1_T56 (SEQ ID NO:45. Table 86 below describes the starting and ending position of this segment on each transcript.

TABLE 86
Segment location on transcripts

| | Segment | |
|---|---|---|
| | starting | Segment |
| Transcript name | position | ending position |
| HUMHPA1B_PEA_1_T1 (SEQ ID | 389 | 411 |
| NO: 34) | | |
| HUMHPA1B_PEA_1_T4 (SEQ ID | 1326 | 1348 |
| NO: 35) | | |
| HUMHPA1B_PEA_1_T6 (SEQ ID | 1324 | 1346 |
| NO: 36) | | |
| HUMHPA1B_PEA_1_T7 (SEQ ID | 1489 | 1511 |
| NO: 37) | | |
| HUMHPA1B_PEA_1_T12 (SEQ ID | 389 | 411 |
| NO: 38) | | |
| HUMHPA1B_PEA_1_T16 (SEQ ID | 297 | 319 |
| NO: 39) | | |
| HUMHPA1B_PEA_1_T20 (SEQ ID | 245 | 267 |
| NO: 41) | | |
| HUMHPA1B_PEA_1_T56 (SEQ ID | 389 | 411 |
| NO: 45) | | |

Segment cluster HUMHPA1B_PEA_{—}1_node_{—}44 (SEQ ID NO:88) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T20 (SEQ ID NO:41) and HUMHPA1B_PEA_{—}1_T56 (SEQ ID NO:45). Table 87 below describes the starting and ending position of this segment on each transcript.

TABLE 87
Segment location on transcripts

| | Segment | |
|---|---|---|
| | starting | Segment |
| Transcript name | position | ending position |
| HUMHPA1B_PEA_1_T1 (SEQ ID | 412 | 424 |
| NO: 34) | | |
| HUMHPA1B_PEA_1_T4 (SEQ ID | 1349 | 1361 |
| NO: 35) | | |
| HUMHPA1B_PEA_1_T6 (SEQ ID | 1347 | 1359 |
| NO: 36) | | |
| HUMHPA1B_PEA_1_T7 (SEQ ID | 1512 | 1524 |
| NO: 37) | | |
| HUMHPA1B_PEA_1_T12 (SEQ ID | 412 | 424 |
| NO: 38) | | |
| HUMHPA1B_PEA_1_T16 (SEQ ID | 320 | 332 |
| NO: 39) | | |
| HUMHPA1B_PEA_1_T20 (SEQ ID | 268 | 280 |
| NO: 41) | | |
| HUMHPA1B_PEA_1_T56 (SEQ ID | 412 | 424 |
| NO: 45) | | |

Segment cluster HUMHPA1B_PEA_{—}1_node_{—}45 (SEQ ID NO:89) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T20 (SEQ ID NO:41), HUMHPA1B_PEA_{—}1_T29 (SEQ ID NO:43) and HUMHPA1B_PEA_{—}1_T56 (SEQ ID NO:45). Table 88 below describes the starting and ending position of this segment on each transcript.

TABLE 88
Segment location on transcripts

| | | Segment |
|---|---|---|
| | Segment | ending |
| Transcript name | starting position | position |
| HUMHPA1B_PEA_1_T1 (SEQ ID | 425 | 436 |
| NO: 34) | | |
| HUMHPA1B_PEA_1_T4 (SEQ ID | 1362 | 1373 |
| NO: 35) | | |
| HUMHPA1B_PEA_1_T6 (SEQ ID | 1360 | 1371 |
| NO: 36) | | |
| HUMHPA1B_PEA_1_T7 (SEQ ID | 1525 | 1536 |
| NO: 37) | | |
| HUMHPA1B_PEA_1_T12 (SEQ ID | 425 | 436 |
| NO: 38) | | |
| HUMHPA1B_PEA_1_T16 (SEQ ID | 333 | 344 |
| NO: 39) | | |
| HUMHPA1B_PEA_1_T20 (SEQ ID | 281 | 292 |
| NO: 41) | | |
| HUMHPA1B_PEA_1_T29 (SEQ ID | 156 | 167 |
| NO: 43) | | |
| HUMHPA1B_PEA_1_T56 (SEQ ID | 425 | 436 |
| NO: 45) | | |

Segment cluster HUMHPA1B_PEA_{—}1_node_{—}46 (SEQ ID NO:90) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T20 (SEQ ID NO:41), HUMHPA1B_PEA_{—}1_T29 (SEQ ID NO:43) and HUMHPA1B_PEA_{—}1_T56 (SEQ ID NO:45). Table 89 below describes the starting and ending position of this segment on each transcript.

TABLE 89
Segment location on transcripts

| | | Segment |
|---|---|---|
| | Segment | ending |
| Transcript name | starting position | position |
| HUMHPA1B_PEA_1_T1 (SEQ ID | 437 | 447 |
| NO: 34) | | |
| HUMHPA1B_PEA_1_T4 (SEQ ID | 1374 | 1384 |
| NO: 35) | | |
| HUMHPA1B_PEA_1_T6 (SEQ ID | 1372 | 1382 |
| NO: 36) | | |
| HUMHPA1B_PEA_1_T7 (SEQ ID | 1537 | 1547 |
| NO: 37) | | |
| HUMHPA1B_PEA_1_T12 (SEQ ID | 437 | 447 |
| NO: 38) | | |
| HUMHPA1B_PEA_1_T16 (SEQ ID | 345 | 355 |
| NO: 39) | | |
| HUMHPA1B_PEA_1_T20 (SEQ ID | 293 | 303 |
| NO: 41) | | |
| HUMHPA1B_PEA_1_T29 (SEQ ID | 168 | 178 |
| NO: 43) | | |
| HUMHPA1B_PEA_1_T56 (SEQ ID | 437 | 447 |
| NO: 45) | | |

Segment cluster HUMHPA1B_PEA_{—}1_node_{—}47 (SEQ ID NO:91) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T20 (SEQ ID NO:41), HUMHPA1B_PEA_{—}1_T29 (SEQ ID NO:43) and HUMHPA1B_PEA_{—}1_T56 (SEQ ID NO:45). Table 90 below describes the starting and ending position of this segment on each transcript.

TABLE 90
Segment location on transcripts

| | | Segment |
|---|---|---|
| | Segment | ending |
| Transcript name | starting position | position |
| HUMHPA1B_PEA_1_T1 (SEQ ID | 448 | 452 |
| NO: 34) | | |
| HUMHPA1B_PEA_1_T4 (SEQ ID | 1385 | 1389 |
| NO: 35) | | |
| HUMHPA1B_PEA_1_T6 (SEQ ID | 1383 | 1387 |
| NO: 36) | | |
| HUMHPA1B_PEA_1_T7 (SEQ ID | 1548 | 1552 |
| NO: 37) | | |
| HUMHPA1B_PEA_1_T12 (SEQ ID | 448 | 452 |
| NO: 38) | | |
| HUMHPA1B_PEA_1_T16 (SEQ ID | 356 | 360 |
| NO: 39) | | |
| HUMHPA1B_PEA_1_T20 (SEQ ID | 304 | 308 |
| NO: 41) | | |
| HUMHPA1B_PEA_1_T29 (SEQ ID | 179 | 183 |
| NO: 43) | | |
| HUMHPA1B_PEA_1_T56 (SEQ ID | 448 | 452 |
| NO: 45) | | |

Segment cluster HUMHPA1B_PEA_{—}1_node_{—}48 (SEQ ID NO:92) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 91 below describes the starting and ending position of this segment on each transcript.

TABLE 91
Segment location on transcripts

| | | Segment |
|---|---|---|
| | Segment | ending |
| Transcript name | starting position | position |
| HUMHPA1B_PEA_1_T1 (SEQ ID | 453 | 473 |
| NO: 34) | | |
| HUMHPA1B_PEA_1_T4 (SEQ ID | 1390 | 1410 |
| NO: 35) | | |
| HUMHPA1B_PEA_1_T6 (SEQ ID | 1388 | 1408 |
| NO: 36) | | |
| HUMHPA1B_PEA_1_T7 (SEQ ID | 1553 | 1573 |
| NO: 37) | | |
| HUMHPA1B_PEA_1_T12 (SEQ ID | 453 | 473 |
| NO: 38) | | |
| HUMHPA1B_PEA_1_T16 (SEQ ID | 361 | 381 |
| NO: 39) | | |
| HUMHPA1B_PEA_1_T19 (SEQ ID | 510 | 530 |
| NO: 40) | | |
| HUMHPA1B_PEA_1_T20 (SEQ ID | 309 | 329 |
| NO: 41) | | |
| HUMHPA1B_PEA_1_T27 (SEQ ID | 333 | 353 |
| NO: 42) | | |
| HUMHPA1B_PEA_1_T29 (SEQ ID | 184 | 204 |
| NO: 43) | | |

Segment cluster HUMHPA1B_PEA_{—}1_node_{—}49 (SEQ ID NO:93) according to the present invention is supported by 105 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38, HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 92 below describes the starting and ending position of this segment on each transcript.

TABLE 92
Segment location on transcripts

| | | Segment |
|---|---|---|
| | Segment | ending |
| Transcript name | starting position | position |
| HUMHPA1B_PEA_1_T1 (SEQ ID | 474 | 511 |
| NO: 34) | | |
| HUMHPA1B_PEA_1_T4 (SEQ ID | 1411 | 1448 |
| NO: 35) | | |
| HUMHPA1B_PEA_1_T6 (SEQ ID | 1409 | 1446 |
| NO: 36) | | |
| HUMHPA1B_PEA_1_T7 (SEQ ID | 1574 | 1611 |
| NO: 37) | | |
| HUMHPA1B_PEA_1_T12 (SEQ ID | 474 | 511 |
| NO: 38) | | |
| HUMHPA1B_PEA_1_T16 (SEQ ID | 382 | 419 |
| NO: 39) | | |
| HUMHPA1B_PEA_1_T19 (SEQ ID | 531 | 568 |
| NO: 40) | | |
| HUMHPA1B_PEA_1_T20 (SEQ ID | 330 | 367 |
| NO: 41) | | |
| HUMHPA1B_PEA_1_T27 (SEQ ID | 354 | 391 |
| NO: 42) | | |
| HUMHPA1B_PEA_1_T29 (SEQ ID | 205 | 242 |
| NO: 43) | | |

Segment cluster HUMHPA1B_PEA_{—}1_node_{—}50 (SEQ ID NO:94) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 93 below describes the starting and ending position of this segment on each transcript.

TABLE 93
Segment location on transcripts

| | | Segment |
|---|---|---|
| | Segment | ending |
| Transcript name | starting position | position |
| HUMHPA1B_PEA_1_T1 (SEQ ID | 512 | 530 |
| NO: 34) | | |
| HUMHPA1B_PEA_1_T4 (SEQ ID | 1449 | 1467 |
| NO: 35) | | |
| HUMHPA1B_PEA_1_T6 (SEQ ID | 1447 | 1465 |
| NO: 36) | | |
| HUMHPA1B_PEA_1_T7 (SEQ ID | 1612 | 1630 |
| NO: 37) | | |
| HUMHPA1B_PEA_1_T12 (SEQ ID | 512 | 530 |
| NO: 38) | | |
| HUMHPA1B_PEA_1_T16 (SEQ ID | 420 | 438 |
| NO: 39) | | |
| HUMHPA1B_PEA_1_T19 (SEQ ID | 569 | 587 |
| NO: 40) | | |
| HUMHPA1B_PEA_1_T20 (SEQ ID | 368 | 386 |
| NO: 41) | | |
| HUMHPA1B_PEA_1_T27 (SEQ ID | 392 | 410 |
| NO: 42) | | |
| HUMHPA1B_PEA_1_T29 (SEQ ID | 243 | 261 |
| NO: 43) | | |

Segment cluster HUMHPA1B_PEA_{—}1_node_{—}51 (SEQ ID NO:95) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 94 below describes the starting and ending position of this segment on each transcript.

TABLE 94
Segment location on transcripts

| | | Segment |
|---|---|---|
| | Segment | ending |
| Transcript name | starting position | position |
| HUMHPA1B_PEA_1_T1 (SEQ ID | 531 | 549 |
| NO: 34) | | |
| HUMHPA1B_PEA_1_T4 (SEQ ID | 1468 | 1486 |
| NO: 35) | | |
| HUMHPA1B_PEA_1_T6 (SEQ ID | 1466 | 1484 |
| NO: 36) | | |
| HUMHPA1B_PEA_1_T7 (SEQ ID | 1631 | 1649 |
| NO: 37) | | |
| HUMHPA1B_PEA_1_T12 (SEQ ID | 531 | 549 |
| NO: 38) | | |
| HUMHPA1B_PEA_1_T16 (SEQ ID | 439 | 457 |
| NO: 39) | | |
| HUMHPA1B_PEA_1_T19 (SEQ ID | 588 | 606 |
| NO: 40) | | |
| HUMHPA1B_PEA_1_T20 (SEQ ID | 387 | 405 |
| NO: 41) | | |
| HUMHPA1B_PEA_1_T27 (SEQ ID | 411 | 429 |
| NO: 42) | | |
| HUMHPA1B_PEA_1_T29 (SEQ ID | 262 | 280 |
| NO: 43) | | |

Segment cluster HUMHPA1B_PEA_{—}1_node_{—}52 (SEQ ID NO:96) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 95 below describes the starting and ending position of this segment on each transcript.

TABLE 95
Segment location on transcripts

| | | Segment |
|---|---|---|
| | Segment | ending |
| Transcript name | starting position | position |
| HUMHPA1B_PEA_1_T1 (SEQ ID | 550 | 558 |
| NO: 34) | | |
| HUMHPA1B_PEA_1_T4 (SEQ ID | 1487 | 1495 |
| NO: 35) | | |
| HUMHPA1B_PEA_1_T6 (SEQ ID | 1485 | 1493 |
| NO: 36) | | |
| HUMHPA1B_PEA_1_T7 (SEQ ID | 1650 | 1658 |
| NO: 37) | | |
| HUMHPA1B_PEA_1_T12 (SEQ ID | 550 | 558 |
| NO: 38) | | |
| HUMHPA1B_PEA_1_T16 (SEQ ID | 458 | 466 |
| NO: 39) | | |
| HUMHPA1B_PEA_1_T19 (SEQ ID | 607 | 615 |
| NO: 40) | | |
| HUMHPA1B_PEA_1_T20 (SEQ ID | 406 | 414 |
| NO: 41) | | |
| HUMHPA1B_PEA_1_T27 (SEQ ID | 430 | 438 |
| NO: 42) | | |
| HUMHPA1B_PEA_1_T29 (SEQ ID | 281 | 289 |
| NO: 43) | | |

Segment cluster HUMHPA1B_PEA_{—}1_node_{—}53 (SEQ ID NO:97) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37, HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 96 below describes the starting and ending position of this segment on each transcript.

TABLE 96
Segment location on transcripts

| | | Segment |
|---|---|---|
| | Segment | ending |
| Transcript name | starting position | position |
| HUMHPA1B_PEA_1_T1 (SEQ ID | 559 | 567 |
| NO: 34) | | |
| HUMHPA1B_PEA_1_T4 (SEQ ID | 1496 | 1504 |
| NO: 35) | | |
| HUMHPA1B_PEA_1_T6 (SEQ ID | 1494 | 1502 |
| NO: 36) | | |
| HUMHPA1B_PEA_1_T7 (SEQ ID | 1659 | 1667 |
| NO: 37) | | |
| HUMHPA1B_PEA_1_T12 (SEQ ID | 559 | 567 |
| NO: 38) | | |
| HUMHPA1B_PEA_1_T16 (SEQ ID | 467 | 475 |
| NO: 39) | | |
| HUMHPA1B_PEA_1_T19 (SEQ ID | 616 | 624 |
| NO: 40) | | |
| HUMHPA1B_PEA_1_T20 (SEQ ID | 415 | 423 |
| NO: 41) | | |
| HUMHPA1B_PEA_1_T27 (SEQ ID | 439 | 447 |
| NO: 42) | | |
| HUMHPA1B_PEA_1_T29 (SEQ ID | 290 | 298 |
| NO: 43) | | |

Segment cluster HUMHPA1B_PEA_{—}1_node_{—}54 (SEQ ID NO:98) according to the present invention can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42) and HUMHPA1B_PEA_{—}1_T29 (SEQ ID NO:43). Table 97 below describes the starting and ending position of this segment on each transcript.

TABLE 97
Segment location on transcripts

| | | Segment |
|---|---|---|
| | Segment | ending |
| Transcript name | starting position | position |
| HUMHPA1B_PEA_1_T1 (SEQ ID | 568 | 574 |
| NO: 34) | | |
| HUMHPA1B_PEA_1_T4 (SEQ ID | 1505 | 1511 |
| NO: 35) | | |
| HUMHPA1B_PEA_1_T6 (SEQ ID | 1503 | 1509 |
| NO: 36) | | |
| HUMHPA1B_PEA_1_T7 (SEQ ID | 1668 | 1674 |
| NO: 37) | | |
| HUMHPA1B_PEA_1_T12 (SEQ ID | 568 | 574 |
| NO: 38) | | |
| HUMHPA1B_PEA_1_T16 (SEQ ID | 476 | 482 |
| NO: 39) | | |
| HUMHPA1B_PEA_1_T19 (SEQ ID | 625 | 631 |
| NO: 40) | | |
| HUMHPA1B_PEA_1_T20 (SEQ ID | 424 | 430 |
| NO: 41) | | |
| HUMHPA1B_PEA_1_T27 (SEQ ID | 448 | 454 |
| NO: 42) | | |
| HUMHPA1B_PEA_1_T29 (SEQ ID | 299 | 305 |
| NO: 43) | | |

Segment cluster HUMHPA1B_PEA_{—}1_node_{—}55 (SEQ ID NO:99) according to the present invention is supported by 113 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40, HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 98 below describes the starting and ending position of this segment on each transcript.

TABLE 98
Segment location on transcripts

| | Segment | Segment |
|---|---|---|
| | starting | ending |
| Transcript name | position | position |
| HUMHPA1B_PEA_1_T1 (SEQ ID | 575 | 616 |
| NO: 34) | | |
| HUMHPA1B_PEA_1_T4 (SEQ ID | 1512 | 1553 |
| NO: 35) | | |
| HUMHPA1B_PEA_1_T6 (SEQ ID | 1510 | 1551 |
| NO: 36) | | |
| HUMHPA1B_PEA_1_T7 (SEQ ID | 1675 | 1716 |
| NO: 37) | | |
| HUMHPA1B_PEA_1_T12 (SEQ ID | 575 | 616 |
| NO: 38) | | |
| HUMHPA1B_PEA_1_T16 (SEQ ID | 483 | 524 |
| NO: 39) | | |
| HUMHPA1B_PEA_1_T19 (SEQ ID | 632 | 673 |
| NO: 40) | | |
| HUMHPA1B_PEA_1_T20 (SEQ ID | 431 | 472 |
| NO: 41) | | |
| HUMHPA1B_PEA_1_T27 (SEQ ID | 455 | 496 |
| NO: 42) | | |
| HUMHPA1B_PEA_1_T29 (SEQ ID | 306 | 347 |
| NO: 43) | | |

Segment cluster HUMHPA1B_PEA_{—}1_node_{—}56 (SEQ ID NO:100) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 99 below describes the starting and ending position of this segment on each transcript.

TABLE 99
Segment location on transcripts

| | Segment | Segment |
|---|---|---|
| | starting | ending |
| Transcript name | position | position |
| HUMHPA1B_PEA_1_T1 (SEQ ID | 617 | 622 |
| NO: 34) | | |
| HUMHPA1B_PEA_1_T4 (SEQ ID | 1554 | 1559 |
| NO: 35) | | |
| HUMHPA1B_PEA_1_T6 (SEQ ID | 1552 | 1557 |
| NO: 36) | | |
| HUMHPA1B_PEA_1_T7 (SEQ ID | 1717 | 1722 |
| NO: 37) | | |
| HUMHPA1B_PEA_1_T12 (SEQ ID | 617 | 622 |
| NO: 38) | | |
| HUMHPA1B_PEA_1_T16 (SEQ ID | 525 | 530 |
| NO: 39) | | |
| HUMHPA1B_PEA_1_T19 (SEQ ID | 674 | 679 |
| NO: 40) | | |
| HUMHPA1B_PEA_1_T20 (SEQ ID | 473 | 478 |
| NO: 41) | | |
| HUMHPA1B_PEA_1_T27 (SEQ ID | 497 | 502 |
| NO: 42) | | |
| HUMHPA1B_PEA_1_T29 (SEQ ID | 348 | 353 |
| NO: 43) | | |

Segment cluster HUMHPA1B_PEA_{—}1_node_{—}57 (SEQ ID NO:101) according to the present invention is supported by 110 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 100 below describes the starting and ending position of this segment on each transcript.

TABLE 100
Segment location on transcripts

| | Segment | Segment |
|---|---|---|
| | starting | ending |
| Transcript name | position | position |
| HUMHPA1B_PEA_1_T1 (SEQ ID | 623 | 649 |
| NO: 34) | | |
| HUMHPA1B_PEA_1_T4 (SEQ ID | 1560 | 1586 |
| NO: 35) | | |
| HUMHPA1B_PEA_1_T6 (SEQ ID | 1558 | 1584 |
| NO: 36) | | |
| HUMHPA1B_PEA_1_T7 (SEQ ID | 1723 | 1749 |
| NO: 37) | | |
| HUMHPA1B_PEA_1_T12 (SEQ ID | 623 | 649 |
| NO: 38) | | |
| HUMHPA1B_PEA_1_T16 (SEQ ID | 531 | 557 |
| NO: 39) | | |
| HUMHPA1B_PEA_1_T19 (SEQ ID | 680 | 706 |
| NO: 40) | | |
| HUMHPA1B_PEA_1_T20 (SEQ ID | 479 | 505 |
| NO: 41) | | |
| HUMHPA1B_PEA_1_T27 (SEQ ID | 503 | 529 |
| NO: 42) | | |
| HUMHPA1B_PEA_1_T29 (SEQ ID | 354 | 380 |
| NO: 43) | | |

Segment cluster HUMHPA1B_PEA_{—}1_node_{—}58 (SEQ ID NO:102) according to the present invention is supported by 108 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40, HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 101 below describes the starting and ending position of this segment on each transcript.

TABLE 101
Segment location on transcripts

| | Segment | Segment |
|---|---|---|
| | starting | ending |
| Transcript name | position | position |
| HUMHPA1B_PEA_1_T1 (SEQ ID | 650 | 684 |
| NO: 34) | | |
| HUMHPA1B_PEA_1_T4 (SEQ ID | 1587 | 1621 |
| NO: 35) | | |
| HUMHPA1B_PEA_1_T6 (SEQ ID | 1585 | 1619 |
| NO: 36) | | |
| HUMHPA1B_PEA_1_T7 (SEQ ID | 1750 | 1784 |
| NO: 37) | | |
| HUMHPA1B_PEA_1_T12 (SEQ ID | 650 | 684 |
| NO: 38) | | |
| HUMHPA1B_PEA_1_T16 (SEQ ID | 558 | 592 |
| NO: 39) | | |
| HUMHPA1B_PEA_1_T19 (SEQ ID | 707 | 741 |
| NO: 40) | | |
| HUMHPA1B_PEA_1_T20 (SEQ ID | 506 | 540 |
| NO: 41) | | |
| HUMHPA1B_PEA_1_T27 (SEQ ID | 530 | 564 |
| NO: 42) | | |
| HUMHPA1B_PEA_1_T29 (SEQ ID | 381 | 415 |
| NO: 43) | | |

Segment cluster HUMHPA1B_PEA_{—}1 node_{—}59 (SEQ ID NO:103) according to the present invention can be found in the following transcript(s): HUMHPA1B_PEA_{—}1_T1 (SEQ ID NO:34), HUMHPA1B_PEA_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 102 below describes the starting and ending position of this segment on each transcript.

TABLE 102
Segment location on transcripts

| | Segment | Segment |
|---|---|---|
| | starting | ending |
| Transcript name | position | position |
| HUMHPA1B_PEA_1_T1 (SEQ ID | 685 | 692 |
| NO: 34) | | |
| HUMHPA1B_PEA_1_T4 (SEQ ID | 1622 | 1629 |
| NO: 35) | | |
| HUMHPA1B_PEA_1_T6 (SEQ ID | 1620 | 1627 |
| NO: 36) | | |
| HUMHPA1B_PEA_1_T7 (SEQ ID | 1785 | 1792 |
| NO: 37) | | |
| HUMHPA1B_PEA_1_T12 (SEQ ID | 685 | 692 |
| NO: 38) | | |
| HUMHPA1B_PEA_1_T16 (SEQ ID | 593 | 600 |
| NO: 39) | | |
| HUMHPA1B_PEA_1_T19 (SEQ ID | 742 | 749 |
| NO: 40) | | |
| HUMHPA1B_PEA_1_T20 (SEQ ID | 541 | 548 |
| NO: 41) | | |
| HUMHPA1B_PEA_1_T27 (SEQ ID | 565 | 572 |
| NO: 42) | | |
| HUMHPA1B_PEA_1_T29 (SEQ ID | 416 | 423 |
| NO: 43) | | |

Segment cluster HUMHPA1_B_PEA_{—}1_node_{—}60 (SEQ ID NO:104) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43. Table 103 below describes the starting and ending position of this segment on each transcript.

TABLE 103
Segment location on transcripts

| | Segment | Segment |
|---|---|---|
| | starting | ending |
| Transcript name | position | position |
| HUMHPA1B_PEA_1_T1 (SEQ ID | 693 | 700 |
| NO: 34) | | |
| HUMHPA1B_PEA_1_T4 (SEQ ID | 1630 | 1637 |
| NO: 35) | | |
| HUMHPA1B_PEA_1_T6 (SEQ ID | 1628 | 1635 |
| NO: 36) | | |
| HUMHPA1B_PEA_1_T7 (SEQ ID | 1793 | 1800 |
| NO: 37) | | |
| HUMHPA1B_PEA_1_T12 (SEQ ID | 693 | 700 |
| NO: 38) | | |
| HUMHPA1B_PEA_1_T16 (SEQ ID | 601 | 608 |
| NO: 39) | | |
| HUMHPA1B_PEA_1_T19 (SEQ ID | 750 | 757 |
| NO: 40) | | |
| HUMHPA1B_PEA_1_T20 (SEQ ID | 549 | 556 |
| NO: 41) | | |
| HUMHPA1B_PEA_1_T27 (SEQ ID | 573 | 580 |
| NO: 42) | | |
| HUMHPA1B_PEA_1_T29 (SEQ ID | 424 | 431 |
| NO: 43) | | |

Segment cluster HUMHPA1B_PEA_{—}1_node_{—}61 (SEQ ID NO:105) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43. Table 104 below describes the starting and ending position of this segment on each transcript.

TABLE 104
Segment location on transcripts

| | Segment | Segment |
|---|---|---|
| | starting | ending |
| Transcript name | position | position |
| HUMHPA1B_PEA_1_T1 (SEQ ID | 701 | 712 |
| NO: 34) | | |
| HUMHPA1B_PEA_1_T4 (SEQ ID | 1638 | 1649 |
| NO: 35) | | |
| HUMHPA1B_PEA_1_T6 (SEQ ID | 1636 | 1647 |
| NO: 36) | | |
| HUMHPA1B_PEA_1_T7 (SEQ ID | 1801 | 1812 |
| NO: 37) | | |
| HUMHPA1B_PEA_1_T12 (SEQ ID | 701 | 712 |
| NO: 38) | | |
| HUMHPA1B_PEA_1_T16 (SEQ ID | 609 | 620 |
| NO: 39) | | |
| HUMHPA1B_PEA_1_T19 (SEQ ID | 758 | 769 |
| NO: 40) | | |
| HUMHPA1B_PEA_1_T20 (SEQ ID | 557 | 568 |
| NO: 41) | | |
| HUMHPA1B_PEA_1_T27 (SEQ ID | 581 | 592 |
| NO: 42) | | |
| HUMHPA1B_PEA_1_T29 (SEQ ID | 432 | 443 |
| NO: 43) | | |

Segment cluster HUMHPA1B_PEA_{—}1_node_{—}62 (SEQ ID NO:106) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 105 below describes the starting and ending position of this segment on each transcript.

TABLE 105
Segment location on transcripts

| | Segment | Segment |
|---|---|---|
| | starting | ending |
| Transcript name | position | position |
| HUMHPA1B_PEA_1_T1 (SEQ ID | 713 | 723 |
| NO: 34) | | |
| HUMHPA1B_PEA_1_T4 (SEQ ID | 1650 | 1660 |
| NO: 35) | | |
| HUMHPA1B_PEA_1_T6 (SEQ ID | 1648 | 1658 |
| NO: 36) | | |
| HUMHPA1B_PEA_1_T7 (SEQ ID | 1813 | 1823 |
| NO: 37) | | |
| HUMHPA1B_PEA_1_T12 (SEQ ID | 713 | 723 |
| NO: 38) | | |
| HUMHPA1B_PEA_1_T16 (SEQ ID | 621 | 631 |
| NO: 39) | | |
| HUMHPA1B_PEA_1_T19 (SEQ ID | 770 | 780 |
| NO: 40) | | |
| HUMHPA1B_PEA_1_T20 (SEQ ID | 569 | 579 |
| NO: 41) | | |
| HUMHPA1B_PEA_1_T27 (SEQ ID | 593 | 603 |
| NO: 42) | | |
| HUMHPA1B_PEA_1_T29 (SEQ ID | 444 | 454 |
| NO: 43) | | |

Segment cluster HUMHPA1B_PEA_{—}1_node_{—}63 (SEQ ID NO:107) according to the present invention is supported by 112 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40, HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43 Table 106 below describes the starting and ending position of this segment on each transcript.

TABLE 106
Segment location on transcripts

| | Segment | Segment |
|---|---|---|
| | starting | ending |
| Transcript name | position | position |
| HUMHPA1B_PEA_1_T1 (SEQ ID | 724 | 767 |
| NO: 34) | | |
| HUMHPA1B_PEA_1_T4 (SEQ ID | 1661 | 1704 |
| NO: 35) | | |
| HUMHPA1B_PEA_1_T6 (SEQ ID | 1659 | 1702 |
| NO: 36) | | |
| HUMHPA1B_PEA_1_T7 (SEQ ID | 1824 | 1867 |
| NO: 37) | | |
| HUMHPA1B_PEA_1_T12 (SEQ ID | 724 | 767 |
| NO: 38) | | |
| HUMHPA1B_PEA_1_T16 (SEQ ID | 632 | 675 |
| NO: 39) | | |
| HUMHPA1B_PEA_1_T19 (SEQ ID | 781 | 824 |
| NO: 40) | | |
| HUMHPA1B_PEA_1_T20 (SEQ ID | 580 | 623 |
| NO: 41) | | |
| HUMHPA1B_PEA_1_T27 (SEQ ID | 604 | 647 |
| NO: 42) | | |
| HUMHPA1B_PEA_1_T29 (SEQ ID | 455 | 498 |
| NO: 43) | | |

Segment cluster HUMHPA1B_PEA_{—}1_node_{—}64 (SEQ ID NO:108) according to the present invention is supported by 115 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 107 below describes the starting and ending position of this segment on each transcript.

TABLE 107
Segment location on transcripts

| | Segment | Segment |
|---|---|---|
| | starting | ending |
| Transcript name | position | position |
| HUMHPA1B_PEA_1_T1 (SEQ ID | 768 | 815 |
| NO: 34) | | |
| HUMHPA1B_PEA_1_T4 (SEQ ID | 1705 | 1752 |
| NO: 35) | | |
| HUMHPA1B_PEA_1_T6 (SEQ ID | 1703 | 1750 |
| NO: 36) | | |
| HUMHPA1B_PEA_1_T7 (SEQ ID | 1868 | 1915 |
| NO: 37) | | |
| HUMHPA1B_PEA_1_T12 (SEQ ID | 768 | 815 |
| NO: 38) | | |
| HUMHPA1B_PEA_1_T16 (SEQ ID | 676 | 723 |
| NO: 39) | | |
| HUMHPA1B_PEA_1_T19 (SEQ ID | 825 | 872 |
| NO: 40) | | |
| HUMHPA1B_PEA_1_T20 (SEQ ID | 624 | 671 |
| NO: 41) | | |
| HUMHPA1B_PEA_1_T27 (SEQ ID | 648 | 695 |
| NO: 42) | | |
| HUMHPA1B_PEA_1_T29 (SEQ ID | 499 | 546 |
| NO: 43) | | |

Segment cluster HUMHPA1B_PEA_{—}1_node_{—}65 (SEQ ID NO:109) according to the present invention can be found in the following transcript(s): HUMHPA1B_PEA_{—}1_T1 (SEQ ID NO:34), HUMHPA1B_PEA_{—}1_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 108 below describes the starting and ending position of this segment on each transcript.

TABLE 108
Segment location on transcripts

| | | Segment |
|---|---|---|
| | Segment | ending |
| Transcript name | starting position | position |
| HUMHPA1B_PEA_1_T1 (SEQ ID | 816 | 837 |
| NO: 34) | | |
| HUMHPA1B_PEA_1_T4 (SEQ ID | 1753 | 1774 |
| NO: 35) | | |
| HUMHPA1B_PEA_1_T6 (SEQ ID | 1751 | 1772 |
| NO: 36) | | |
| HUMHPA1B_PEA_1_T7 (SEQ ID | 1916 | 1937 |
| NO: 37) | | |
| HUMHPA1B_PEA_1_T12 (SEQ ID | 816 | 837 |
| NO: 38) | | |
| HUMHPA1B_PEA_1_T16 (SEQ ID | 724 | 745 |
| NO: 39) | | |
| HUMHPA1B_PEA_1_T19 (SEQ ID | 873 | 894 |
| NO: 40) | | |
| HUMHPA1B_PEA_1_T20 (SEQ ID | 672 | 693 |
| NO: 41) | | |
| HUMHPA1B_PEA_1_T27 (SEQ ID | 696 | 717 |
| NO: 42) | | |
| HUMHPA1B_PEA_1_T29 (SEQ ID | 547 | 568 |
| NO: 43) | | |

Segment cluster HUMHPA1B_PEA_{—}1_node_{—}66 (SEQ ID NO:110) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43). Table 109 below describes the starting and ending position of this segment on each transcript.

TABLE 109
Segment location on transcripts

| | | Segment |
|---|---|---|
| | Segment | ending |
| Transcript name | starting position | position |
| HUMHPA1B_PEA_1_T1 (SEQ ID | 838 | 846 |
| NO: 34) | | |
| HUMHPA1B_PEA_1_T4 (SEQ ID | 1775 | 1783 |
| NO: 35) | | |
| HUMHPA1B_PEA_1_T6 (SEQ ID | 1773 | 1781 |
| NO: 36) | | |
| HUMHPA1B_PEA_1_T7 (SEQ ID | 1938 | 1946 |
| NO: 37) | | |
| HUMHPA1B_PEA_1_T12 (SEQ ID | 838 | 846 |
| NO: 38) | | |
| HUMHPA1B_PEA_1_T16 (SEQ ID | 746 | 754 |
| NO: 39) | | |
| HUMHPA1B_PEA_1_T19 (SEQ ID | 895 | 903 |
| NO: 40) | | |
| HUMHPA1B_PEA_1_T20 (SEQ ID | 694 | 702 |
| NO: 41) | | |
| HUMHPA1B_PEA_1_T27 (SEQ ID | 718 | 726 |
| NO: 42) | | |
| HUMHPA1B_PEA_1_T29 (SEQ ID | 569 | 577 |
| NO: 43) | | |

Segment cluster HUMHPA1B_PEA_{—}1_node_{—}67 (SEQ ID NO:111) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42) and HUMHPA1B_PEA_{—}1_T29 (SEQ ID NO:43). Table 110 below describes the starting and ending position of this segment on each transcript.

TABLE 110
Segment location on transcripts

| | | Segment |
|---|---|---|
| | Segment | ending |
| Transcript name | starting position | position |
| HUMHPA1B_PEA_1_T1 (SEQ ID | 847 | 856 |
| NO: 34) | | |
| HUMHPA1B_PEA_1_T4 (SEQ ID | 1784 | 1793 |
| NO: 35) | | |
| HUMHPA1B_PEA_1_T6 (SEQ ID | 1782 | 1791 |
| NO: 36) | | |
| HUMHPA1B_PEA_1_T7 (SEQ ID | 1947 | 1956 |
| NO: 37) | | |
| HUMHPA1B_PEA_1_T12 (SEQ ID | 847 | 856 |
| NO: 38) | | |
| HUMHPA1B_PEA_1_T16 (SEQ ID | 755 | 764 |
| NO: 39) | | |
| HUMHPA1B_PEA_1_T19 (SEQ ID | 904 | 913 |
| NO: 40) | | |
| HUMHPA1B_PEA_1_T20 (SEQ ID | 703 | 712 |
| NO: 41) | | |
| HUMHPA1B_PEA_1_T27 (SEQ ID | 727 | 736 |
| NO: 42) | | |
| HUMHPA1B_PEA_1_T29 (SEQ ID | 578 | 587 |
| NO: 43) | | |

Segment cluster HUMHPA1B_PEA_{—}1_node_{—}69 (SEQ ID NO:112) according to the present invention is supported by 107 libraries. The number of libraries was determined as previously described. This segment can be found in the following transcript(s): HUMHPA1B_PEA_{—}1_T1 (SEQ ID NO:34, HUMHPA1B_PEA_{—}1_T4 (SEQ ID NO:35), HUMHPA1B_PEA_{—}1_T6 (SEQ ID NO:36, HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42) and HUMHPA1B_PEA_{—}1_T29 (SEQ ID NO:43 Table 111 below describes the starting and ending position of this segment on each transcript.

TABLE 111
Segment location on transcripts

| | | Segment |
|---|---|---|
| | Segment | ending |
| Transcript name | starting position | position |
| HUMHPA1B_PEA_1_T1 (SEQ ID | 857 | 883 |
| NO: 34) | | |
| HUMHPA1B_PEA_1_T4 (SEQ ID | 1794 | 1820 |
| NO: 35) | | |
| HUMHPA1B_PEA_1_T6 (SEQ ID | 1792 | 1818 |
| NO: 36) | | |
| HUMHPA1B_PEA_1_T7 (SEQ ID | 1957 | 1983 |
| NO: 37) | | |
| HUMHPA1B_PEA_1_T12 (SEQ ID | 857 | 883 |
| NO: 38) | | |
| HUMHPA1B_PEA_1_T16 (SEQ ID | 765 | 791 |
| NO: 39) | | |
| HUMHPA1B_PEA_1_T19 (SEQ ID | 914 | 940 |
| NO: 40) | | |
| HUMHPA1B_PEA_1_T20 (SEQ ID | 713 | 739 |
| NO: 41) | | |
| HUMHPA1B_PEA_1_T27 (SEQ ID | 737 | 763 |
| NO: 42) | | |
| HUMHPA1B_PEA_1_T29 (SEQ ID | 588 | 614 |
| NO: 43) | | |

Segment cluster HUMHPA1B_PEA_{—}1_node_{—}70 (SEQ ID NO:113) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 112 below describes the starting and ending position of this segment on each transcript.

TABLE 112
Segment location on transcripts

| | | Segment |
|---|---|---|
| | Segment | ending |
| Transcript name | starting position | position |
| HUMHPA1B_PEA_1_T1 (SEQ ID | 884 | 890 |
| NO: 34) | | |
| HUMHPA1B_PEA_1_T4 (SEQ ID | 1821 | 1827 |
| NO: 35) | | |
| HUMHPA1B_PEA_1_T6 (SEQ ID | 1819 | 1825 |
| NO: 36) | | |
| HUMHPA1B_PEA_1_T7 (SEQ ID | 1984 | 1990 |
| NO: 37) | | |
| HUMHPA1B_PEA_1_T12 (SEQ ID | 884 | 890 |
| NO: 38) | | |
| HUMHPA1B_PEA_1_T16 (SEQ ID | 792 | 798 |
| NO: 39) | | |
| HUMHPA1B_PEA_1_T19 (SEQ ID | 941 | 947 |
| NO: 40) | | |
| HUMHPA1B_PEA_1_T20 (SEQ ID | 740 | 746 |
| NO: 41) | | |
| HUMHPA1B_PEA_1_T27 (SEQ ID | 764 | 770 |
| NO: 42) | | |
| HUMHPA1B_PEA_1_T29 (SEQ ID | 615 | 621 |
| NO: 43) | | |
| HUMHPA1B_PEA_1_T55 (SEQ ID | 333 | 339 |
| NO: 44) | | |

Segment cluster HUMHPA1B_PEA_{—}1_node_{—}71 (SEQ ID NO:114) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 113 below describes the starting and ending position of this segment on each transcript.

TABLE 113
Segment location on transcripts

| | | Segment |
|---|---|---|
| | Segment | ending |
| Transcript name | starting position | position |
| HUMHPA1B_PEA_1_T1 (SEQ ID | 891 | 899 |
| NO: 34) | | |
| HUMHPA1B_PEA_1_T4 (SEQ ID | 1828 | 1836 |
| NO: 35) | | |
| HUMHPA1B_PEA_1_T6 (SEQ ID | 1826 | 1834 |
| NO: 36) | | |
| HUMHPA1B_PEA_1_T7 (SEQ ID | 1991 | 1999 |
| NO: 37) | | |
| HUMHPA1B_PEA_1_T12 (SEQ ID | 891 | 899 |
| NO: 38) | | |
| HUMHPA1B_PEA_1_T16 (SEQ ID | 799 | 807 |
| NO: 39) | | |
| HUMHPA1B_PEA_1_T19 (SEQ ID | 948 | 956 |
| NO: 40) | | |
| HUMHPA1B_PEA_1_T20 (SEQ ID | 747 | 755 |
| NO: 41) | | |
| HUMHPA1B_PEA_1_T27 (SEQ ID | 771 | 779 |
| NO: 42) | | |
| HUMHPA1B_PEA_1_T29 (SEQ ID | 622 | 630 |
| NO: 43) | | |
| HUMHPA1B_PEA_1_T55 (SEQ ID | 340 | 348 |
| NO: 44) | | |

Segment cluster HUMHPA1B_PEA_{—}1_node_{—}72 (SEQ ID NO:115) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 114 below describes the starting and ending position of this segment on each transcript.

TABLE 114
Segment location on transcripts

| | | Segment |
|---|---|---|
| | Segment | ending |
| Transcript name | starting position | position |
| HUMHPA1B_PEA_1_T1 (SEQ ID | 900 | 903 |
| NO: 34) | | |
| HUMHPA1B_PEA_1_T4 (SEQ ID | 1837 | 1840 |
| NO: 35) | | |
| HUMHPA1B_PEA_1_T6 (SEQ ID | 1835 | 1838 |
| NO: 36) | | |
| HUMHPA1B_PEA_1_T7 (SEQ ID | 2000 | 2003 |
| NO: 37) | | |
| HUMHPA1B_PEA_1_T12 (SEQ ID | 900 | 903 |
| NO: 38) | | |
| HUMHPA1B_PEA_1_T16 (SEQ ID | 808 | 811 |
| NO: 39) | | |
| HUMHPA1B_PEA_1_T19 (SEQ ID | 957 | 960 |
| NO: 40) | | |
| HUMHPA1B_PEA_1_T20 (SEQ ID | 756 | 759 |
| NO: 41) | | |
| HUMHPA1B_PEA_1_T27 (SEQ ID | 780 | 783 |
| NO: 42) | | |
| HUMHPA1B_PEA_1_T29 (SEQ ID | 631 | 634 |
| NO: 43) | | |
| HUMHPA1B_PEA_1_T55 (SEQ ID | 349 | 352 |
| NO: 44) | | |

Segment cluster HUMHPA1B_PEA_{—}1_node_{—}73 (SEQ ID NO:116) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 115 below describes the starting and ending position of this segment on each transcript.

TABLE 115
Segment location on transcripts

| | | Segment |
|---|---|---|
| | Segment | ending |
| Transcript name | starting position | position |
| HUMHPA1B_PEA_1_T1 (SEQ ID | 904 | 920 |
| NO: 34) | | |
| HUMHPA1B_PEA_1_T4 (SEQ ID | 1841 | 1857 |
| NO: 35) | | |
| HUMHPA1B_PEA_1_T6 (SEQ ID | 1839 | 1855 |
| NO: 36) | | |
| HUMHPA1B_PEA_1_T7 (SEQ ID | 2004 | 2020 |
| NO: 37) | | |
| HUMHPA1B_PEA_1_T12 (SEQ ID | 904 | 920 |
| NO: 38) | | |
| HUMHPA1B_PEA_1_T16 (SEQ ID | 812 | 828 |
| NO: 39) | | |
| HUMHPA1B_PEA_1_T19 (SEQ ID | 961 | 977 |
| NO: 40) | | |
| HUMHPA1B_PEA_1_T20 (SEQ ID | 760 | 776 |
| NO: 41) | | |
| HUMHPA1B_PEA_1_T27 (SEQ ID | 784 | 800 |
| NO: 42) | | |
| HUMHPA1B_PEA_1_T29 (SEQ ID | 635 | 651 |
| NO: 43) | | |
| HUMHPA1B_PEA_1_T55 (SEQ ID | 353 | 369 |
| NO: 44) | | |

Segment cluster HUMHPA1B_PEA_{—}1_node_{—}74 (SEQ ID NO:117) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 116 below describes the starting and ending position of this segment on each transcript.

TABLE 116
Segment location on transcripts

| | | Segment |
|---|---|---|
| | Segment | ending |
| Transcript name | starting position | position |
| HUMHPA1B_PEA_1_T1 (SEQ ID | 921 | 928 |
| NO: 34) | | |
| HUMHPA1B_PEA_1_T4 (SEQ ID | 1858 | 1865 |
| NO: 35) | | |
| HUMHPA1B_PEA_1_T6 (SEQ ID | 1856 | 1863 |
| NO: 36) | | |
| HUMHPA1B_PEA_1_T7 (SEQ ID | 2021 | 2028 |
| NO: 37) | | |
| HUMHPA1B_PEA_1_T12 (SEQ ID | 921 | 928 |
| NO: 38) | | |
| HUMHPA1B_PEA_1_T16 (SEQ ID | 829 | 836 |
| NO: 39) | | |
| HUMHPA1B_PEA_1_T19 (SEQ ID | 978 | 985 |
| NO: 40) | | |
| HUMHPA1B_PEA_1_T20 (SEQ ID | 777 | 784 |
| NO: 41) | | |
| HUMHPA1B_PEA_1_T27 (SEQ ID | 801 | 808 |
| NO: 42) | | |
| HUMHPA1B_PEA_1_T29 (SEQ ID | 652 | 659 |
| NO: 43) | | |
| HUMHPA1B_PEA_1_T55 (SEQ ID | 370 | 377 |
| NO: 44) | | |

Segment cluster HUMHPA1B_PEA_{—}1_node_{—}75 (SEQ ID NO:118) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T9 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 117 below describes the starting and ending position of this segment on each transcript.

TABLE 117
Segment location on transcripts

| | | Segment |
|---|---|---|
| | Segment | ending |
| Transcript name | starting position | position |
| HUMHPA1B_PEA_1_T1 (SEQ ID | 929 | 939 |
| NO: 34) | | |
| HUMHPA1B_PEA_1_T4 (SEQ ID | 1866 | 1876 |
| NO: 35) | | |
| HUMHPA1B_PEA_1_T6 (SEQ ID | 1864 | 1874 |
| NO: 36) | | |
| HUMHPA1B_PEA_1_T7 (SEQ ID | 2029 | 2039 |
| NO: 37) | | |
| HUMHPA1B_PEA_1_T12 (SEQ ID | 929 | 939 |
| NO: 38) | | |
| HUMHPA1B_PEA_1_T16 (SEQ ID | 837 | 847 |
| NO: 39) | | |
| HUMHPA1B_PEA_1_T19 (SEQ ID | 986 | 996 |
| NO: 40) | | |
| HUMHPA1B_PEA_1_T20 (SEQ ID | 785 | 795 |
| NO: 41) | | |
| HUMHPA1B_PEA_1_T27 (SEQ ID | 809 | 819 |
| NO: 42) | | |
| HUMHPA1B_PEA_1_T29 (SEQ ID | 660 | 670 |
| NO: 43) | | |
| HUMHPA1B_PEA_1_T55 (SEQ ID | 378 | 388 |
| NO: 44) | | |

Segment cluster HUMHPA1B_PEA_{—}1_node_{—}76 (SEQ ID NO:119) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39, HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43) and HUMHPA1B_PEA_{—}1_T55 (SEQ ID NO:44). Table 118 below describes the starting and ending position of this segment on each transcript.

TABLE 118
Segment location on transcripts

| | Segment | Segment |
|---|---|---|
| | starting | ending |
| Transcript name | position | position |
| HUMHPA1B_PEA_1_T1 (SEQ ID | 940 | 960 |
| NO: 34) | | |
| HUMHPA1B_PEA_1_T4 (SEQ ID | 1877 | 1897 |
| NO: 35) | | |
| HUMHPA1B_PEA_1_T6 (SEQ ID | 1875 | 1895 |
| NO: 36) | | |
| HUMHPA1B_PEA_1_T7 (SEQ ID | 2040 | 2060 |
| NO: 37) | | |
| HUMHPA1B_PEA_1_T12 (SEQ ID | 940 | 960 |
| NO: 38) | | |
| HUMHPA1B_PEA_1_T16 (SEQ ID | 848 | 868 |
| NO: 39) | | |
| HUMHPA1B_PEA_1_T19 (SEQ ID | 997 | 1017 |
| NO: 40) | | |
| HUMHPA1B_PEA_1_T20 (SEQ ID | 796 | 816 |
| NO: 41) | | |
| HUMHPA1B_PEA_1_T27 (SEQ ID | 820 | 840 |
| NO: 42) | | |
| HUMHPA1B_PEA_1_T29 (SEQ ID | 671 | 691 |
| NO: 43) | | |
| HUMHPA1B_PEA_1_T55 (SEQ ID | 389 | 409 |
| NO: 44) | | |

Segment cluster HUMHPA1B_PEA_{—}1_node_{—}77 (SEQ ID NO:120) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45. Table 119 below describes the starting and ending position of this segment on each transcript.

TABLE 119
Segment location on transcripts

| | Segment | Segment |
|---|---|---|
| | starting | ending |
| Transcript name | position | position |
| HUMHPA1B_PEA_1_T1 (SEQ ID | 961 | 979 |
| NO: 34) | | |
| HUMHPA1B_PEA_1_T4 (SEQ ID | 1898 | 1916 |
| NO: 35) | | |
| HUMHPA1B_PEA_1_T6 (SEQ ID | 1896 | 1914 |
| NO: 36) | | |
| HUMHPA1B_PEA_1_T7 (SEQ ID | 2061 | 2079 |
| NO: 37) | | |
| HUMHPA1B_PEA_1_T12 (SEQ ID | 961 | 979 |
| NO: 38) | | |
| HUMHPA1B_PEA_1_T16 (SEQ ID | 869 | 887 |
| NO: 39) | | |
| HUMHPA1B_PEA_1_T19 (SEQ ID | 1018 | 1036 |
| NO: 40) | | |
| HUMHPA1B_PEA_1_T20 (SEQ ID | 817 | 835 |
| NO: 41) | | |
| HUMHPA1B_PEA_1_T27 (SEQ ID | 841 | 859 |
| NO: 42) | | |
| HUMHPA1B_PEA_1_T29 (SEQ ID | 692 | 710 |
| NO: 43) | | |
| HUMHPA1B_PEA_1_T55 (SEQ ID | 410 | 428 |
| NO: 44) | | |
| HUMHPA1B_PEA_1_T56 (SEQ ID | 453 | 471 |
| NO: 45) | | |

Segment cluster HUMHPA1B_PEA_{—}1_node_{—}78 (SEQ ID NO:121) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 120 below describes the starting and ending position of this segment on each transcript.

TABLE 120
Segment location on transcripts

| | Segment | Segment |
|---|---|---|
| | starting | ending |
| Transcript name | position | position |
| HUMHPA1B_PEA_1_T1 (SEQ ID | 980 | 988 |
| NO: 34) | | |
| HUMHPA1B_PEA_1_T4 (SEQ ID | 1917 | 1925 |
| NO: 35) | | |
| HUMHPA1B_PEA_1_T6 (SEQ ID | 1915 | 1923 |
| NO: 36) | | |
| HUMHPA1B_PEA_1_T7 (SEQ ID | 2080 | 2088 |
| NO: 37) | | |
| HUMHPA1B_PEA_1_T12 (SEQ ID | 980 | 988 |
| NO: 38) | | |
| HUMHPA1B_PEA_1_T16 (SEQ ID | 888 | 896 |
| NO: 39) | | |
| HUMHPA1B_PEA_1_T19 (SEQ ID | 1037 | 1045 |
| NO: 40) | | |
| HUMHPA1B_PEA_1_T20 (SEQ ID | 836 | 844 |
| NO: 41) | | |
| HUMHPA1B_PEA_1_T27 (SEQ ID | 860 | 868 |
| NO: 42) | | |
| HUMHPA1B_PEA_1_T29 (SEQ ID | 711 | 719 |
| NO: 43) | | |
| HUMHPA1B_PEA_1_T55 (SEQ ID | 429 | 437 |
| NO: 44) | | |
| HUMHPA1B_PEA_1_T56 (SEQ ID | 472 | 480 |
| NO: 45) | | |

Segment cluster HUMHPA1B_PEA_{—}1_node_{—}79 (SEQ ID NO:122) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 121 below describes the starting and ending position of this segment on each transcript.

TABLE 121
Segment location on transcripts

| | Segment | Segment |
|---|---|---|
| | starting | ending |
| Transcript name | position | position |
| HUMHPA1B_PEA_1_T1 (SEQ ID | 989 | 993 |
| NO: 34) | | |
| HUMHPA1B_PEA_1_T4 (SEQ ID | 1926 | 1930 |
| NO: 35) | | |
| HUMHPA1B_PEA_1_T6 (SEQ ID | 1924 | 1928 |
| NO: 36) | | |
| HUMHPA1B_PEA_1_T7 (SEQ ID | 2089 | 2093 |
| NO: 37) | | |
| HUMHPA1B_PEA_1_T12 (SEQ ID | 989 | 993 |
| NO: 38) | | |
| HUMHPA1B_PEA_1_T16 (SEQ ID | 897 | 901 |
| NO: 39) | | |
| HUMHPA1B_PEA_1_T19 (SEQ ID | 1046 | 1050 |
| NO: 40) | | |
| HUMHPA1B_PEA_1_T20 (SEQ ID | 845 | 849 |
| NO: 41) | | |
| HUMHPA1B_PEA_1_T27 (SEQ ID | 869 | 873 |
| NO: 42) | | |
| HUMHPA1B_PEA_1_T29 (SEQ ID | 720 | 724 |
| NO: 43) | | |
| HUMHPA1B_PEA_1_T55 (SEQ ID | 438 | 442 |
| NO: 44) | | |
| HUMHPA1B_PEA_1_T56 (SEQ ID | 481 | 485 |
| NO: 45) | | |

Segment cluster HUMHPA1B_PEA_{—}1_node_{—}80 (SEQ ID NO:123) according to the present invention can be found in the following transcript(s): HUMHPA1B_PEA_{—}1_T1 (SEQ ID NO:34), HUMHPA1B_PEA I_T4 (SEQ ID NO:35, HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 122 below describes the starting and ending position of this segment on each transcript.

TABLE 122
Segment location on transcripts

| | Segment | Segment |
|---|---|---|
| | starting | ending |
| Transcript name | position | position |
| HUMHPA1B_PEA_1_T1 (SEQ ID | 994 | 1005 |
| NO: 34) | | |
| HUMHPA1B_PEA_1_T4 (SEQ ID | 1931 | 1942 |
| NO: 35) | | |
| HUMHPA1B_PEA_1_T6 (SEQ ID | 1929 | 1940 |
| NO: 36) | | |
| HUMHPA1B_PEA_1_T7 (SEQ ID | 2094 | 2105 |
| NO: 37) | | |
| HUMHPA1B_PEA_1_T12 (SEQ ID | 994 | 1005 |
| NO: 38) | | |
| HUMHPA1B_PEA_1_T16 (SEQ ID | 902 | 913 |
| NO: 39) | | |
| HUMHPA1B_PEA_1_T19 (SEQ ID | 1051 | 1062 |
| NO: 40) | | |
| HUMHPA1B_PEA_1_T20 (SEQ ID | 850 | 861 |
| NO: 41) | | |
| HUMHPA1B_PEA_1_T27 (SEQ ID | 874 | 885 |
| NO: 42) | | |
| HUMHPA1B_PEA_1_T29 (SEQ ID | 725 | 736 |
| NO: 43) | | |
| HUMHPA1B_PEA_1_T55 (SEQ ID | 443 | 454 |
| NO: 44) | | |
| HUMHPA1B_PEA_1_T56 (SEQ ID | 486 | 497 |
| NO: 45) | | |

Segment cluster HUMHPA1B_PEA_{—}1_node_{—}81 (SEQ ID NO:124) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41, HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 123 below describes the starting and ending position of this segment on each transcript.

TABLE 123
Segment location on transcripts

| | Segment | Segment |
|---|---|---|
| | starting | ending |
| Transcript name | position | position |
| HUMHPA1B_PEA_1_T1 (SEQ ID | 1006 | 1017 |
| NO: 34) | | |
| HUMHPA1B_PEA_1_T4 (SEQ ID | 1943 | 1954 |
| NO: 35) | | |
| HUMHPA1B_PEA_1_T6 (SEQ ID | 1941 | 1952 |
| NO: 36) | | |
| HUMHPA1B_PEA_1_T7 (SEQ ID | 2106 | 2117 |
| NO: 37) | | |
| HUMHPA1B_PEA_1_T12 (SEQ ID | 1006 | 1017 |
| NO: 38) | | |
| HUMHPA1B_PEA_1_T16 (SEQ ID | 914 | 925 |
| NO: 39) | | |
| HUMHPA1B_PEA_1_T19 (SEQ ID | 1063 | 1074 |
| NO: 40) | | |
| HUMHPA1B_PEA_1_T20 (SEQ ID | 862 | 873 |
| NO: 41) | | |
| HUMHPA1B_PEA_1_T27 (SEQ ID | 886 | 897 |
| NO: 42) | | |
| HUMHPA1B_PEA_1_T29 (SEQ ID | 737 | 748 |
| NO: 43) | | |
| HUMHPA1B_PEA_1_T55 (SEQ ID | 455 | 466 |
| NO: 44) | | |
| HUMHPA1B_PEA_1_T56 (SEQ ID | 498 | 509 |
| NO: 45) | | |

Segment cluster HUMHPA1B_PEA_{—}1_node_{—}82 (SEQ ID NO:125) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 124 below describes the starting and ending position of this segment on each transcript.

TABLE 124
Segment location on transcripts

| | Segment | Segment |
|---|---|---|
| | starting | ending |
| Transcript name | position | position |
| HUMHPA1B_PEA_1_T1 (SEQ ID | 1018 | 1029 |
| NO: 34) | | |
| HUMHPA1B_PEA_1_T4 (SEQ ID | 1955 | 1966 |
| NO: 35) | | |
| HUMHPA1B_PEA_1_T6 (SEQ ID | 1953 | 1964 |
| NO: 36) | | |
| HUMHPA1B_PEA_1_T7 (SEQ ID | 2118 | 2129 |
| NO: 37) | | |
| HUMHPA1B_PEA_1_T12 (SEQ ID | 1018 | 1029 |
| NO: 38) | | |
| HUMHPA1B_PEA_1_T16 (SEQ ID | 926 | 937 |
| NO: 39) | | |
| HUMHPA1B_PEA_1_T19 (SEQ ID | 1075 | 1086 |
| NO: 40) | | |
| HUMHPA1B_PEA_1_T20 (SEQ ID | 874 | 885 |
| NO: 41) | | |
| HUMHPA1B_PEA_1_T27 (SEQ ID | 898 | 909 |
| NO: 42) | | |
| HUMHPA1B_PEA_1_T29 (SEQ ID | 749 | 760 |
| NO: 43) | | |
| HUMHPA1B_PEA_1_T55 (SEQ ID | 467 | 478 |
| NO: 44) | | |
| HUMHPA1B_PEA_1_T56 (SEQ ID | 510 | 521 |
| NO: 45) | | |

Segment cluster HUMHPA1B_PEA_{—}1_node_{—}83 (SEQ ID NO:126) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45. Table 125 below describes the starting and ending position of this segment on each transcript.

TABLE 125
Segment location on transcripts

| | Segment | Segment |
|---|---|---|
| | starting | ending |
| Transcript name | position | position |
| HUMHPA1B_PEA_1_T1 (SEQ ID | 1030 | 1040 |
| NO: 34) | | |
| HUMHPA1B_PEA_1_T4 (SEQ ID | 1967 | 1977 |
| NO: 35) | | |
| HUMHPA1B_PEA_1_T6 (SEQ ID | 1965 | 1975 |
| NO: 36) | | |
| HUMHPA1B_PEA_1_T7 (SEQ ID | 2130 | 2140 |
| NO: 37) | | |
| HUMHPA1B_PEA_1_T12 (SEQ ID | 1030 | 1040 |
| NO: 38) | | |
| HUMHPA1B_PEA_1_T16 (SEQ ID | 938 | 948 |
| NO: 39) | | |
| HUMHPA1B_PEA_1_T19 (SEQ ID | 1087 | 1097 |
| NO: 40) | | |
| HUMHPA1B_PEA_1_T20 (SEQ ID | 886 | 896 |
| NO: 41) | | |
| HUMHPA1B_PEA_1_T27 (SEQ ID | 910 | 920 |
| NO: 42) | | |
| HUMHPA1B_PEA_1_T29 (SEQ ID | 761 | 771 |
| NO: 43) | | |
| HUMHPA1B_PEA_1_T55 (SEQ ID | 479 | 489 |
| NO: 44) | | |
| HUMHPA1B_PEA_1_T56 (SEQ ID | 522 | 532 |
| NO: 45) | | |

Segment cluster HUMHPA1B_PEA_{—}1_node_{—}84 (SEQ ID NO:127) according to the present invention is supported by 104 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41) HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 126 below describes the starting and ending position of this segment on each transcript.

TABLE 126
Segment location on transcripts

| | Segment | Segment |
|---|---|---|
| | starting | ending |
| Transcript name | position | position |
| HUMHPA1B_PEA_1_T1 (SEQ ID | 1041 | 1071 |
| NO: 34) | | |
| HUMHPA1B_PEA_1_T4 (SEQ ID | 1978 | 2008 |
| NO: 35) | | |
| HUMHPA1B_PEA_1_T6 (SEQ ID | 1976 | 2006 |
| NO: 36) | | |
| HUMHPA1B_PEA_1_T7 (SEQ ID | 2141 | 2171 |
| NO: 37) | | |
| HUMHPA1B_PEA_1_T12 (SEQ ID | 1041 | 1071 |
| NO: 38) | | |
| HUMHPA1B_PEA_1_T16 (SEQ ID | 949 | 979 |
| NO: 39) | | |
| HUMHPA1B_PEA_1_T19 (SEQ ID | 1098 | 1128 |
| NO: 40) | | |
| HUMHPA1B_PEA_1_T20 (SEQ ID | 897 | 927 |
| NO: 41) | | |
| HUMHPA1B_PEA_1_T27 (SEQ ID | 921 | 951 |
| NO: 42) | | |
| HUMHPA1B_PEA_1_T29 (SEQ ID | 772 | 802 |
| NO: 43) | | |
| HUMHPA1B_PEA_1_T55 (SEQ ID | 490 | 520 |
| NO: 44) | | |
| HUMHPA1B_PEA_1_T56 (SEQ ID | 533 | 563 |
| NO: 45) | | |

Segment cluster HUMHPA1B_PEA_{—}1_node_{—}85 (SEQ ID NO:128) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43, HUMHPA1B_PEA_{—}1_T55 (SEQ ID NO:44) and HUMHPA1B_PEA_{—}1_T56 (SEQ ID NO:45). Table 127 below describes the starting and ending position of this segment on each transcript.

TABLE 127
Segment location on transcripts

| | Segment | Segment |
|---|---|---|
| | starting | ending |
| Transcript name | position | position |
| HUMHPA1B_PEA_1_T1 (SEQ ID | 1072 | 1078 |
| NO: 34) | | |
| HUMHPA1B_PEA_1_T4 (SEQ ID | 2009 | 2015 |
| NO: 35) | | |
| HUMHPA1B_PEA_1_T6 (SEQ ID | 2007 | 2013 |
| NO: 36) | | |
| HUMHPA1B_PEA_1_T7 (SEQ ID | 2172 | 2178 |
| NO: 37) | | |
| HUMHPA1B_PEA_1_T12 (SEQ ID | 1072 | 1078 |
| NO: 38) | | |
| HUMHPA1B_PEA_1_T16 (SEQ ID | 980 | 986 |
| NO: 39) | | |
| HUMHPA1B_PEA_1_T19 (SEQ ID | 1129 | 1135 |
| NO: 40) | | |
| HUMHPA1B_PEA_1_T20 (SEQ ID | 928 | 934 |
| NO: 41) | | |
| HUMHPA1B_PEA_1_T27 (SEQ ID | 952 | 958 |
| NO: 42) | | |
| HUMHPA1B_PEA_1_T29 (SEQ ID | 803 | 809 |
| NO: 43) | | |
| HUMHPA1B_PEA_1_T55 (SEQ ID | 521 | 527 |
| NO: 44) | | |
| HUMHPA1B_PEA_1_T56 (SEQ ID | 564 | 570 |
| NO: 45) | | |

Segment cluster HUMHPA1B_PEA_{—}1_node_{—}86 (SEQ ID NO:129) according to the present invention can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38), HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45). Table 128 below describes the starting and ending position of this segment on each transcript.

TABLE 128
Segment location on transcripts

| | | |
|---|---|---|
| | Segment | |
| | starting | Segment |
| Transcript name | position | ending position |

| | | |
|---|---|---|
| HUMHPA1B_PEA_1_T1 (SEQ ID | 1079 | 1090 |
| NO: 34) | | |
| HUMHPA1B_PEA_1_T4 (SEQ ID | 2016 | 2027 |
| NO: 35) | | |
| HUMHPA1B_PEA_1_T6 (SEQ ID | 2014 | 2025 |
| NO: 36) | | |
| HUMHPA1B_PEA_1_T7 (SEQ ID | 2179 | 2190 |
| NO: 37) | | |
| HUMHPA1B_PEA_1_T12 (SEQ ID | 1079 | 1090 |
| NO: 38) | | |
| HUMHPA1B_PEA_1_T16 (SEQ ID | 987 | 998 |
| NO: 39) | | |
| HUMHPA1B_PEA_1_T19 (SEQ ID | 1136 | 1147 |
| NO: 40) | | |
| HUMHPA1B_PEA_1_T20 (SEQ ID | 935 | 946 |
| NO: 41) | | |
| HUMHPA1B_PEA_1_T27 (SEQ ID | 959 | 970 |
| NO: 42) | | |
| HUMHPA1B_PEA_1_T29 (SEQ ID | 810 | 821 |
| NO: 43) | | |
| HUMHPA1B_PEA_1_T55 (SEQ ID | 528 | 539 |
| NO: 44) | | |
| HUMHPA1B_PEA_1_T56 (SEQ ID | 571 | 582 |
| NO: 45) | | |

Segment cluster HUMHPA1B_PEA_{—}1_node_{—}87 (SEQ ID NO:130) according to the present invention is supported by 102 libraries. The number of libraries was determined as previously described. This segment can be found in the following transcript(s): HUMHPA1B_PEA_{—}1_T1 (SEQ ID NO:34), HUMHPA1B_PEA_{—}1_T4 (SEQ ID NO:35), HUMHPA1B_PEA_{—}1_T6 (SEQ ID NO:36), HUMHPA1B_PEA_{—}1_T7 (SEQ ID NO:37), HUMHPA1B_PEA_{—}1_T12 (SEQ ID NO:38, HUMHPA1B_PEA_{—}1_T16 (SEQ ID NO:39), HUMHPA1B_PEA_{—}1_T19 (SEQ ID NO:40), HUMHPA1B_PEA_{—}1_T20 (SEQ ID NO:41), HUMHPA1B_PEA_{—}1_T27 (SEQ ID NO:42), HUMHPA1B_PEA_{—}1_T29 (SEQ ID NO:43), HUMHPA1B_PEA_{—}1_T55 (SEQ ID NO:44) and HUMHPA1B_PEA_{—}1_T56 (SEQ ID NO:45 Table 129 below describes the starting and ending position of this segment on each transcript.

TABLE 129
Segment location on transcripts

| | Segment | |
|---|---|---|
| | starting | Segment |
| Transcript name | position | ending position |
| HUMHPA1B_PEA_1_T1 (SEQ ID | 1091 | 1154 |
| NO: 34) | | |
| HUMHPA1B_PEA_1_T4 (SEQ ID | 2028 | 2091 |
| NO: 35) | | |
| HUMHPA1B_PEA_1_T6 (SEQ ID | 2026 | 2089 |
| NO: 36) | | |
| HUMHPA1B_PEA_1_T7 (SEQ ID | 2191 | 2254 |
| NO: 37) | | |
| HUMHPA1B_PEA_1_T12 (SEQ ID | 1091 | 1154 |
| NO: 38) | | |
| HUMHPA1B_PEA_1_T16 (SEQ ID | 999 | 1062 |
| NO: 39) | | |
| HUMHPA1B_PEA_1_T19 (SEQ ID | 1148 | 1211 |
| NO: 40) | | |
| HUMHPA1B_PEA_1_T20 (SEQ ID | 947 | 1010 |
| NO: 41) | | |
| HUMHPA1B_PEA_1_T27 (SEQ ID | 971 | 1034 |
| NO: 42) | | |
| HUMHPA1B_PEA_1_T29 (SEQ ID | 822 | 885 |
| NO: 43) | | |
| HUMHPA1B_PEA_1_T55 (SEQ ID | 540 | 603 |
| NO: 44) | | |
| HUMHPA1B_PEA_1_T56 (SEQ ID | 583 | 646 |
| NO: 45) | | |

Variant protein alignment to the previously known protein:

| Sequence name: HPT_HUMAN | | | |
|---|---|---|---|
| Sequence documentation: | | | |
| Alignment of: HUMHPA1B_PEA_1_P61 (SEQ ID NO: 133) × HPT_HUMAN (SEQ ID NO: 131) .. | | | |
| Alignment segment 1/1: | | | |
| Quality: | 3336.00 | Escore: | 0 |
| Matching length: | 347 | Total length: | 406 |
| Matching Percent Similarity: | 100.00 | Matching Percent Identity: | 100.00 |
| Total Percent Similarity: | 85.47 | Total Percent Identity: | 85.47 |
| Gaps: | 1 | | |

| Alignment: | | |
|---|---|---|
| | . . . . . | |
| 1 | MSALGAVIALLLWGQLFAVDSGNDVTDI...................... | 28 |
| | \|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 29 | .....................................ADDGCPKPPEIAH | 41 |
| | \|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . . | |
| 42 | GYVEHSVRYQCKNYYKLRTEGDGVYTLNNEKQWINKAVGDKLPECEAVCG | 91 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | GYVEHSVRYQCKNYYKLRTEGDGVYTLNNEKQWINKAVGDKLPECEAVCG | 150 |
| | . . . . . | |
| 92 | KPKNPANPVQRILGGHLDAKGSFPWQAKMVSHHNLTTGATLINEQWLLTT | 141 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | KPKNPANPVQRILGGHLDAKGSFPWQAKMVSHHNLTTGATLINEQWLLTT | 200 |
| | . . . . . | |
| 142 | AKNLFLNHSENATAKDIAPTLTLYVGKKQLVEIEKVVLHPNYSQVDIGLI | 191 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | AKNLFLNHSENATAKDIAPTLTLYVGKKQLVEIEKVVLHPNYSQVDIGLI | 250 |
| | . . . . . | |
| 192 | KLKQKVSVNERVMPICLPSKDYAEVGRVGYVSGWGRNANFKFTDHLKYVM | 241 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KLKQKVSVNERVMPICLPSKDYAEVGRVGYVSGWGRNANFKFTDHLKYVM | 300 |
| | . . . . . | |
| 242 | LPVADQDQCIRHYEGSTVPEKKTPKSPVGVQPILNEHTFCAGMSKYQEDT | 291 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | LPVADQDQCIRHYEGSTVPEKKTPKSPVGVQPILNEHTFCAGMSKYQEDT | 350 |
| | . . . . . | |
| 292 | CYGDAGSAFAVHDLEEDTWYATGILSFDKSCAVAEYGVYVKVTSIQDWVQ | 341 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | CYGDAGSAFAVHDLEEDTWYATGILSFDKSCAVAEYGVYVKVTSIQDWVQ | 400 |
| 342 | KTIAEN | 347 |
| | \|\|\|\|\|\| | |
| 401 | KTIAEN | 406 |

| Sequence name: HPT_HUMAN | | | |
|---|---|---|---|
| Sequence documentation: | | | |
| Alignment of: HUMHPA1B_PEA_1_P62 (SEQ ID NO: 134) × HPT_HUMAN (SEQ ID NO: 131) .. | | | |
| Alignment segment 1/1: | | | |
| Quality: | 630.00 | Escore: | 0 |
| Matching length: | 64 | Total length: | 64 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . | |
| 51 | QCKNYYKLRTEGDG | 64 |
| | \|\|\|\|\|\|\|\|\|\|\|\|\|\| | |
| 51 | QCKNYYKLRTEGDG | 64 |

| Sequence name: HPT_HUMAN | | | |
|---|---|---|---|
| Sequence documentation: | | | |
| Alignment of: HUMHPA1B_PEA_1_P64 (SEQ ID NO: 135) × HPT_HUMAN (SEQ ID NO: 131) .. | | | |
| Alignment segment 1/1: | | | |
| Quality: | 1236.00 | Escore: | 0 |
| Matching length: | 123 | Total length: | 123 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPEPPEIAHGYVEHSVRY | 50 |
| | . . . . . | |
| 51 | QCKNYYKLRTEGDGVYTLNDKKQWINKAVGDKLPECEADDGCPKPPEIAH | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . | |
| 101 | GYVEHSVRYQCKNYYKLRTEGDG | 123 |
| | \|\|\|\|\|\|\|\|\|\|\|\|\|\|\|\|\|\|\|\|\|\|\| | |
| 101 | GYVEHSVRYQCKNYYKLRTEGDG | 123 |

| Sequence name: HPT_HUMAN (SEQ ID NO: 131) | | | |
|---|---|---|---|
| Sequence documentation: | | | |
| Alignment of: HUMHPA1B_PEA_1_P65 (SEQ ID NO: 136) × HPT_HUMAN (SEQ ID NO: 131) .. | | | |
| Alignment segment 1/1: | | | |
| Quality: | 1479.00 | Escore: | 0 |
| Matching length: | 147 | Total length: | 147 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 51 | QCKNYYKLRTEGDGVYTLNDKKQWINKAVGDKLPECEADDGCPKPPEIAH | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . | |
| 101 | GYVEHSVRYQCKNYYKLRTEGDGVYTLNNEKQWINKAVGDKLPECEA | 147 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | GYVEHSVRYQCKNYYKLRTEGDGVYTLNNEKQWINKAVGDKLPECEA | 147 |

| Sequence name: HPT_HUMAN | | | |
|---|---|---|---|
| Sequence documentation: | | | |
| Alignment of: HUMHPA1B_PEA_1_P68 (SEQ ID NO: 137) × HPT_HUMAN (SEQ ID NO: 131) .. | | | |
| Alignment segment 1/1: | | | |
| Quality: | 3335.00 | Escore: | 0 |
| Matching length: | 347 | Total length: | 406 |
| Matching Percent Similarity: | 100.00 | Matching Percent Identity: | 100.00 |
| Total Percent Similarity: | 85.47 | Total Percent Identity: | 85.47 |
| Gaps: | 1 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 51 | QCKNYYKLRTEGDGVYTLNDK............................. | 71 |
| | \|\|\|\|\|\|\|\|\|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . . | |
| 72 | ..............................KQWINKAVGDKLPECEAVCG | 91 |
| | \|\|\|\|\|\|\|\|\|\|\|\|\|\|\|\|\|\|\|\| | |
| 101 | GYVEHSVRYQCKNYYKLRTEGDGVYTLNNEKQWINKAVGDKLPECEAVCG | 150 |
| | . . . . . | |
| 92 | KPKNPANPVQRILGGHLDAKGSFPWQAKMVSHHNLTTGATLINEQWLLTT | 141 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | KPKNPANPVQRILGGHLDAKGSFPWQAKMVSHHNLTTGATLINEQWLLTT | 200 |
| | . . . . . | |
| 142 | AKNLFLNHSENATAKDIAPTLTLYVGKKQLVEIEKVVLHPNYSQVDIGLI | 191 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | AKNLFLNHSENATAKDIAPTLTLYVGKKQLVEIEKVVLHPNYSQVDIGLI | 250 |
| | . . . . . | |
| 192 | KLKQKVSVNERVMPICLPSKDYAEVGRVGYVSGWGRNANFKFTDHLKYVM | 241 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KLKQKVSVNERVMPICLPSKDYAEVGRVGYVSGWGRNANFKFTDHLKYVM | 300 |
| | . . . . . | |
| 242 | LPVADQDQCIRHYEGSTVPEKKTPKSPVGVQPILNEHTFCAGMSKYQEDT | 291 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | LPVADQDQCIRHYEGSTVPEKKTPKSPVGVQPILNEHTFCAGMSKYQEDT | 350 |
| | . . . . . | |
| 292 | CYGDAGSAFAVHDLEEDTWYATGILSFDKSCAVAEYGVYVKVTSIQDWVQ | 341 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | CYGDAGSAFAVHDLEEDTWYATGILSFDKSCAVAEYGVYVKVTSIQDWVQ | 400 |
| 342 | KTIAEN | 347 |
| | \|\|\|\|\|\| | |
| 401 | KTIAEN | 406 |

| Sequence name: HPT_HUMAN (SEQ ID NO: 131) | | | |
|---|---|---|---|
| Sequence documentation: | | | |
| Alignment of: HUMHPA1B_PEA_1_P72 (SEQ ID NO: 138) × HPT_HUMAN (SEQ ID NO: 131) | | | |
| .. | | | |
| Alignment segment 1/1: | | | |
| Quality: | 621.00 | Escore: | 0 |
| Matching length: | 63 | Total length: | 63 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . | |
| 51 | QCKNYYKLRTEGD | 63 |
| | \|\|\|\|\|\|\|\|\|\|\|\|\| | |
| 51 | QCKNYYKLRTEGD | 63 |

| Sequence name: HPT_HUMAN | | | |
|---|---|---|---|
| Sequence documentation: | | | |
| Alignment of: HUMHPA1B_PEA_1_P75 (SEQ ID NO: 139) × HPT_HUMAN (SEQ ID NO: 131) | | | |
| .. | | | |
| Alignment segment 1/1: | | | |
| Quality: | 3534.00 | Escore: | 0 |
| Matching length: | 366 | Total length: | 406 |
| Matching Percent Similarity: | 100.00 | Matching Percent Identity: | 100.00 |
| Total Percent Similarity: | 90.15 | Total Percent Identity: | 90.15 |
| Gaps: | 1 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 51 | QCKNYYKLRTEGDGVYTLNDKKQWINKAVGDKLPECEADDGCPKPPEIAH | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . . | |
| 101 | GYVEHSVRYQCKNYYKLRTEGDGVYTLNNEKQWINKAVGDKLPECEA... | 147 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | GYVEHSVRYQCKNYYKLRTEGDGVYTLNNEKQWINKAVGDKLPECEAVCG | 150 |
| | . . . . . | |
| 148 | .....................................GATLINEQWLLTT | 160 |
| | \|\|\|\|\|\|\|\|\|\|\|\|\| | |
| 151 | KPKNPANPVQRILGGHLDAKGSFPWQAKMVSHHNLTTGATLINEQWLLTT | 200 |
| | . . . . . | |
| 161 | AKNLFLNHSENATAKDIAPTLTLYVGKKQLVEIEKVVLHPNYSQVDIGLI | 210 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | AKNLFLNHSENATAKDIAPTLTLYVGRKQLVEIEKVVLHPNYSQVDIGLI | 250 |
| | . . . . . | |
| 211 | KLKQKVSVNERVMPICLPSKDYAEVGRVGYVSGWGRNANFKFTDHLKYVM | 260 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KLKQKVSVNERVMPICLPSKDYAEVGRVGYVSGWGRNANFKFTDHLKYVM | 300 |
| | . . . . . | |
| 261 | LPVADQDQCIRHYEGSTVPEKKTPKSPVGVQPILNEHTFCAGMSKYQEDT | 310 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | LPVADQDQCIRHYEGSTVPEKKTPKSPVGVQPILNEHTFCAGMSKYQEDT | 350 |
| | . . . . . | |
| 311 | CYGDAGSAFAVHDLEEDTWYATGILSFDKSCAVAEYGVYVKVTSIQDWVQ | 360 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | CYGDAGSAFAVHDLEEDTWYATGILSFDKSCAVAEYGVYVKVTSIQDWVQ | 400 |
| 361 | KTIAEN | 366 |
| | \|\|\|\|\|\| | |
| 401 | KTIAEN | 406 |

| Sequence name: HPT_HUMAN (SEQ ID NO: 131) | | | |
|---|---|---|---|
| Sequence documentation: | | | |
| Alignment of: HUMHPA1B_PEA_1_P76 (SEQ ID NO: 140) × HPT_HUMAN (SEQ ID NO: 131) | | | |
| .. | | | |
| Alignment segment 1/1: | | | |
| Quality: | 2834.00 | Escore: | 0 |
| Matching length: | 299 | Total length: | 406 |
| Matching Percent Similarity: | 100.00 | Matching Percent Identity: | 99.67 |
| Total Percent Similarity: | 73.65 | Total Percent Identity: | 73.40 |
| Gaps: | 1 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 51 | Q................................................. | 51 |
| | \| | |
| 51 | QCKNYYKLRTEGDGVYTLNDKKQWINKAVGDKLPECEADDGCPKPPEIAH | 100 |
| | . . . . . | |
| 51 | .................................................. | 51 |
| 101 | GYVEHSVRYQCKNYYKLRTEGDGVYTLNNEKQWINKAVGDKLPECEAVCG | 150 |
| | . . . . . | |
| 52 | ........LQRILGGHLDAKGSFPWQAKMVSHHNLTTGATLINEQWLLTT | 93 |
| | :\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | KPKNPANPVQRILGGHLDAKGSFPWQAKMVSHHNLTTGATLINEQWLLTT | 200 |
| | . . . . . | |
| 94 | AKNLFLNHSENATAKDIAPTLTLYVGKKQLVEIEKVVLHPNYSQVDIGLI | 143 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | AKNLFLNHSENATAKDIAPTLTLYVGKKQLVEIEKVVLHPNYSQVDIGLI | 250 |
| | . . . . . | |
| 144 | KLKQKVSVNERVMPICLPSKDYAEVGRVGYVSGWGRNANFKFTDHLKYVM | 193 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KLKQKVSVNERVMPICLPSKDYAEVGRVGYVSGWGRNANFKFTDHLKYVM | 300 |
| | . . . . . | |
| 194 | LPVADQDQCIRHYEGSTVPEKKTPKSPVGVQPILNEHTFCAGMSKYQEDT | 243 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | LPVADQDQCIRHYEGSTVPEKKTPKSPVGVQPILNEHTFCAGMSKYQEDT | 350 |
| | . . . . . | |
| 244 | CYGDAGSAFAVHDLEEDTWYATGILSFDKSCAVAEYGVYVKVTSIQDWVQ | 293 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | CYGDAGSAFAVHDLEEDTWYATGILSFDKSCAVAEYGVYVKVTSIQDWVQ | 400 |
| 294 | KTIAEN | 299 |
| | \|\|\|\|\|\| | |
| 401 | KTIAEN | 406 |

| Sequence name: HPT_HUMAN | | | |
|---|---|---|---|
| Sequence documentation: | | | |
| Alignment of: HUMHPA1B_PEA_1_P81 (SEQ ID NO: 141) × HPT HUMAN (SEQ ID NO: 131) | | | |
| .. | | | |
| Alignment segment 1/1: | | | |
| Quality: | 2927.00 | Escore: | 0 |
| Matching length: | 307 | Total length: | 406 |
| Matching Percent Similarity: | 100.00 | Matching Percent Identity: | 100.00 |
| Total Percent Similarity: | 75.62 | Total Percent Identity: | 75.62 |
| Gaps: | 1 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 51 | QCKNYYKLRTEGDGVYTLNDKKQWINKAVGDKLPECEA............ | 88 |
| | \|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . . | |
| 88 | .................................................. | 88 |
| 101 | GYVEHSVRYQCKNYYKLRTEGDGVYTLNNEKQWINKAVGDKLPECEAVCG | 150 |
| | . . . . . | |
| 89 | .....................................GATLINEQWLLTT | 101 |
| | \|\|\|\|\|\|\|\|\|\|\|\|\| | |
| 151 | KPKNPANPVQRILGGHLDAKGSFPWQAKMVSHHNLTTGATLINEQWLLTT | 200 |
| | . . . . . | |
| 102 | AKNLFLNHSENATAKDIAPTLTLYVGKKQLVEIEKVVLHPNYSQVDIGLI | 151 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | AKNLFLNHSENATAKDIAPTLTLYVGKKQLVEIEKVVLHPNYSQVDIGLI | 250 |
| | . . . . . | |
| 152 | KLKQKVSVNERVMPICLPSKDYAEVGRVGYVSGWGRNANFKFTDHLKYVM | 201 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KLKQKVSVNERVMPICLPSKDYAEVGRVGYVSGWGRNANFKFTDHLKYVM | 300 |
| | . . . . . | |
| 202 | LPVADQDQCIRHYEGSTVPEKKTPKSPVGVQPILNEHTFCAGMSKYQEDT | 251 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | LPVADQDQCIRHYEGSTVPEKKTPKSPVGVQPILNEHTFCAGMSKYQEDT | 350 |
| | . . . . . | |
| 252 | CYGDAGSAFAVHDLEEDTWYATGILSFDKSCAVAEYGVYVKVTSIQDWVQ | 301 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | CYGDAGSAFAVHDLEEDTWYATGILSFDKSCAVAEYGVYVKVTSIQDWVQ | 400 |
| 302 | KTIAEN | 307 |
| | \|\|\|\|\|\| | |
| 401 | KTIAEN | 406 |

| Sequence name: HPT_HUMAN (SEQ ID NO: 131) | | | |
|---|---|---|---|
| Sequence documentation: | | | |
| Alignment of: HUMHPA1B_PEA_1_P83 (SEQ ID NO: 142) × HPT_HUMAN (SEQ ID NO: 131) | | | |
| .. | | | |
| Alignment segment 1/1: | | | |
| Quality: | 276.00 | Escore: | 0 |
| Matching length: | 30 | Total length: | 30 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . | |
| 1 | MSALGAVIALLLWGQLFAVDSGNDVTDIAD | 30 |
| | \|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIAD | 30 |

| Sequence name: HPT_HUMAN_V1 (SEQ ID NO: 132) | | | |
|---|---|---|---|
| Sequence documentation: | | | |
| Alignment of: HUMHPA1B_PEA_1_P106 (SEQ ID NO: 143) × HPT_HUMAN_V1 (SEQ ID NO: 132) .. | | | |
| Alignment segment 1/1: | | | |
| Quality: | 863.00 | Escore: | 0 |
| Matching length: | 88 | Total length: | 88 |
| Matching Percent Similarity: | 100.00 | Matching Percent Identity: | 98.86 |
| Total Percent Similarity: | 100.00 | Total Percent Identity: | 98.86 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . | |
| 51 | QCKNYYELRTEGDGVYTLNNEKQWINKAVGDKLPECEA | 88 |
| | \|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNNKKQWINKAVGDKLPECEA | 88 |

| Sequence name: HPT_HUMAN (SEQ ID NO: 131) | | | |
|---|---|---|---|
| Sequence documentation: | | | |
| Alignment of: HUMHPA1B_PEA_1_P107 (SEQ ID NO: 144) × HPT_HUMAN (SEQ ID NO: 131) | | | |
| .. | | | |
| Alignment segment 1/1: | | | |
| Quality: | 1181.00 | Escore: | 0 |
| Matching length: | 128 | Total length: | 187 |
| Matching Percent Similarity: | 100.00 | Matching Percent Identity: | 100.00 |
| Total Percent Similarity: | 68.45 | Total Percent Identity: | 68.45 |
| Gaps: | 1 | | |

| Alignment: | | |
|---|---|---|
| | . . . . . | |
| 1 | MSALGAVIALLLWGQLFAVDSGNDVTDI...................... | 28 |
| | \|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . . . | |
| 29 | .....................................ADDGCPKPPEIAH | 41 |
| | \|\|\|\|\|\|\|\|\|\|\|\|\| | |
| 51 | QCKNYYKLRTEGDGVYTLNDKKQWINKAVGDKLPECEADDGCPKPPEIAH | 100 |
| | . . . . . | |
| 42 | GYVEHSVRYQCKNYYKLRTEGDGVYTLNNEKQWINKAVGDKLPECEAVCG | 91 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | GYVEHSVRYQCKNYYKLRTEGDGVYTLNNEKQWINKAVGDKLPECEAVCG | 150 |
| | . . . | |
| 92 | KPKNPANPVQRILGGHLDAKGSFPWQAKMVSHHNLTT | 128 |
| | \|\|\|\|\|\|\|\|\|\|\|\|\|\|\|\|\|\|\|\|\|\|\|\|\|\|\|\|\|\|\|\|\|\|\|\|\| | |
| 151 | KPKNPANPVQRILGGHLDAKGSFPWQAKMVSHHNLTT | 187 |

| Sequence name: HPT_HUMAN (SEQ ID NO: 131) | | | |
|---|---|---|---|
| Sequence documentation: | | | |
| Alignment of: HUMHPA1B_PEA_1_P115 (SEQ ID NO: 145)) × HPT_HUMAN (SEQ ID NO: 131) | | | |
| .. | | | |
| Alignment segment 1/1: | | | |
| Quality: | 872.00 | Escore: | 0 |
| Matching length: | 88 | Total length: | 88 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MSALGAVIALLLWGQLFAVDSGNDVTDIADDGCPKPPEIAHGYVEHSVRY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSALGAVIALLLWGQLFAVDSGNDVTDIADDGCPKPPEIAHGYVEHSVRY | 50 |
| | . . . | |
| 51 | QCKNYYKLRTEGDGVYTLNDKKQWINKAVGDKLPECEA | 88 |
| | \|\|\|\|\|\|\|\|\|\|\|\|\|\|\|\|\|\|\|\|\|\|\|\|\|\|\|\|\|\|\|\|\|\|\|\|\|\| | |
| 51 | QCKNYYKLRTEGDGVYTLNDKKQWINKAVGDKLPECEA | 88 |

### Description for Cluster HSHGFR

Cluster HSHGFR features 5 transcript(s) and 13 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HSHGFR_T1 | 146 |
| HSHGFR_T6 | 147 |
| HSHGFR_T8 | 148 |
| HSHGFR_T13 | 149 |
| HSHGFR_T14 | 150 |

TABLE 2
Segments of interest

| Segment Name | Sequence ID No. |
|---|---|
| HSHGFR_node_2 | 151 |
| HSHGFR_node_3 | 152 |
| HSHGFR_node_6 | 153 |
| HSHGFR_node_11 | 154 |
| HSHGFR_node_15 | 155 |
| HSHGFR_node_16 | 156 |
| HSHGFR_node_18 | 157 |
| HSHGFR_node_22 | 158 |
| HSHGFR_node_24 | 159 |
| HSHGFR_node_8 | 160 |
| HSHGFR_node_10 | 161 |
| HSHGFR_node_14 | 162 |
| HSHGFR_node_20 | 163 |

TABLE 3
Proteins of interest

| Protein Name | Sequence ID No. | Corresponding Transcript(s) |
|---|---|---|
| HSHGFR_P6 | 165 | HSHGFR_T6 (SEQ ID |
| | | NO: 147); HSHGFR_T8 (SEQ |
| | | ID NO: 148) |
| HSHGFR_P11 | 166 | HSHGFR_T13 (SEQ ID |
| | | NO: 149) |
| HSHGFR_P12 | 167 | HSHGFR_T14 (SEQ ID |
| | | NO: 150) |
| HSHGFR_P13 | 168 | HSHGFR_T1 (SEQ ID |
| | | NO: 146) |

These sequences are variants of the known protein Hepatocyte growth factor precursor (SEQ ID NO:164) (SwissProt accession identifier HGF_HUMAN; known also according to the synonyms Scatter factor; SF; Hepatopoeitin A), referred to herein as the previously known protein.

Protein Hepatocyte growth factor precursor (SEQ ID NO:164) is known or believed to have the following function(s): HGF is a potent mitogen for mature parenchymal hepatocyte cells, seems to be an hepatotrophic factor, and acts as growth factor for a broad spectrum of tissues and cell types. It has no detectable protease activity. The sequence for protein Hepatocyte growth factor precursor is given at the end of the application, as “Hepatocyte growth factor precursor amino acid sequence” (SEQ ID NO:164). Known polymorphisms for this sequence are as shown in Table 4.

TABLE 4
Amino acid mutations for Known Protein

| SNP position(s) on | |
|---|---|
| amino acid sequence | Comment |
| 153 | S -> I (in dbSNP: 17566). /FTId = VAR_014570. |
| 32-33 | QR -> HK |
| 78 | K -> N |
| 162-166 | Missing |
| 180 | P -> T |
| 293 | M -> V |
| 300 | L -> M |
| 317 | V -> A |
| 336 | E -> K |
| 387 | H -> N |
| 416 | D -> N |
| 505 | I -> V |
| 509 | V -> I |
| 558 | D -> E |
| 561 | C -> R |
| 592 | D -> N |
| 595 | S -> N |

The previously known protein also has the following indication(s) and/or potential therapeutic use(s): Cancer; Hepatic dysfunction; Buerger's syndrome. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Angiogenesis inhibitor; Hepatocyte growth factor modulator. A therapeutic role for a protein represented by the cluster has been predicted. The cluster was assigned this field because there was information in the drug database or the public databases (e.g., described herein above) that this protein, or part thereof, is used or can be used for a potential therapeutic indication: Hepatoprotective; Hormone; Radio/chemoprotective; Anticancer; Cardiovascular; Hypolipaemic/Antiatherosclerosis.

The following GO Annotation(s) apply to the previously known protein. The following annotation(s) were found: proteolysis and peptidolysis; mitosis, which are annotation(s) related to Biological Process; and chymotrypsin; trypsin; growth factor, which are annotation(s) related to Molecular Function.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

It was found that concentrations of the known protein in the peritoneal fluid of patients with endometriosis were significantly higher than in those without endometriosis and correlated positively with revised American Society of Reproductive Medicine scores (Yoshida et al, J Clin Endocrinol Metab. 2004 February; 89(2):823-32). Variants of this cluster are suitable as diagnostic markers for endometriosis.

As noted above, cluster HSHGFR features 5 transcript(s), which were listed in Table 1 above. These transcript(s) encode for protein(s) which are variant(s) of protein Hepatocyte growth factor precursor (SEQ ID NO:164). A description of each variant protein according to the present invention is now provided.

Variant protein HSHGFR_P6 (SEQ ID NO:165) according to the present invention has an amino acid sequence as given at the end of the application; it is encoded by transcript(s) HSHGFR_T6 (SEQ ID NO:147) and HSHGFR_T8 (SEQ ID NO:148). An alignment is given to the known protein (Hepatocyte growth factor precursor (SEQ ID NO:164)) at the end of the application. One or more alignments to one or more previously published protein sequences are given at the end of the application. A brief description of the relationship of the variant protein according to the present invention to each such aligned protein is as follows:

Comparison report between HSHGFR_P6 (SEQ ID NO:165) and HGF_HUMAN (SEQ ID NO:164):

1. An isolated chimeric polypeptide encoding for HSHGFR_P6 (SEQ ID NO:165), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEHSFLPSSYRGKDLQENYCR NPRGEEGGPWCFTSNPEVRYEVCDIPQCSEVECMTCNGESYRGLMDHTESGKICQRWD HQTPHRHKFLPERYPDKGFDDNYCRNPDGQPRPWCYTLDPHTRWEYCAIKTCA corresponding to amino acids 1-289 of HGF_HUMAN) (SEQ ID NO:164), which also corresponds to amino acids 1-289 of HSHGFR_P6 (SEQ ID NO:165), and a second amino acid sequence being at least 70%, optionally at least 80%, preferably at least 85%, more preferably at least 90% and most preferably at least 95% homologous to a polypeptide having the sequence E corresponding to amino acids 290-290 of HSHGFR_P6 (SEQ ID NO:165), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSHGFR_P6 (SEQ ID NO:165) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein HSHGFR_P6 (SEQ ID NO:165) Sequence provides support for the deduced sequence of this variant protein according to the present invention).

TABLE 5
Amino acid mutations

| SNP position(s) on amino acid | Alternative | |
|---|---|---|
| sequence | amino acid(s) | Previously known SNP? |
| 53 | I -> V | No |
| 58 | K -> R | No |
| 73 | R -> G | No |
| 90 | D -> G | No |
| 94 | K -> E | No |
| 118 | L -> P | No |
| 126 | R -> G | No |
| 162 | F -> L | No |
| 167 | Y -> C | No |
| 210 | E -> G | No |
| 232 | C -> R | No |
| 236 | D -> G | No |
| 244 | K -> | No |
| 250 | Y -> H | No |
| 258 | N -> D | No |

The glycosylation sites of variant protein HSHGFR_P6 (SEQ ID NO:165), as compared to the known protein Hepatocyte growth factor precursor (SEQ ID NO:164), are described in Table 6 (given according to their position(s) on the amino acid sequence in the first column; the second column indicates whether the glycosylation site is present in the variant protein; and the last column indicates whether the position is different on the variant protein).

TABLE 6
Glycosylation site(s)

| Position(s) on known amino | |
|---|---|
| acid sequence | Present in variant protein? |
| 653 | no |
| 476 | no |
| 566 | no |
| 402 | no |
| 294 | no |

The phosphorylation sites of variant protein HSHGFR_P6 (SEQ ID NO:165), as compared to the known protein Hepatocyte growth factor precursor (SEQ ID NO:164), are described in Table 7 (given according to their position(s) on the amino acid sequence in the first column; the second column indicates whether the phosphorylation site is present in the variant protein; and the last column indicates whether the position is different on the variant protein).

TABLE 7
Phosphorylation site(s)

| Position(s) on known amino | Present in | |
|---|---|---|
| acid sequence | variant protein? | Position in variant protein? |
| 32 | yes | 32 |

Variant protein HSHGFR_P6 (SEQ ID NO:165) is encoded by the following transcript(s): HSHGFR_T6 (SEQ ID NO:147) and HSHGFR_T8 (SEQ ID NO:148), for which the sequence(s) is/are given at the end of the application.

The coding portion of transcript HSHGFR_T6 (SEQ ID NO:147) is shown in bold; this coding portion starts at position 229 and ends at position 1098. The transcript also has the following SNPs as listed in Table 8 (given according to their position on the nucleotide sequence, with the alternative nucleic acid listed; the last column indicates whether the SNP is known or not; the presence of known SNPs in variant protein HSHGFR_P6 (SEQ ID NO:165) sequence provides support for the deduced sequence of this variant protein according to the present invention).

TABLE 8
Nucleic acid SNPs

| SNP position on nucleotide | Alternative | |
|---|---|---|
| sequence | nucleic acid | Previously known SNP? |
| 218 | C -> | No |
| 219 | C -> T | No |
| 256 | C -> T | No |
| 385 | A -> G | No |
| 401 | A -> G | No |
| 445 | A -> G | No |
| 497 | A -> G | No |
| 508 | A -> G | No |
| 552 | G -> A | No |
| 561 | A -> G | No |
| 581 | T -> C | No |
| 604 | A -> G | No |
| 712 | T -> C | No |
| 728 | A -> G | No |
| 760 | C -> A | No |
| 780 | A -> G | No |
| 825 | A -> G | No |
| 857 | A -> G | No |
| 922 | T -> C | No |
| 935 | A -> G | No |
| 958 | A -> | No |
| 976 | T -> C | No |
| 1000 | A -> G | No |
| 1059 | C -> T | No |

The coding portion of transcript HSHGFR_T8 (SEQ ID NO:148) is shown in bold; this coding portion starts at position 229 and ends at position 1098. The transcript also has the following SNPs as listed in Table 9 (given according to their position on the nucleotide sequence, with the alternative nucleic acid listed; the last column indicates whether the SNP is known or not; the presence of known SNPs in variant protein HSHGFR_P6 (SEQ ID NO:165) sequence provides support for the deduced sequence of this variant protein according to the present invention).

TABLE 9
Nucleic acid SNPs

| SNP position on nucleotide | | Previously |
|---|---|---|
| sequence | Alternative nucleic acid | known SNP? |
| 218 | C -> | No |
| 219 | C -> T | No |
| 256 | C -> T | No |
| 385 | A -> G | No |
| 401 | A -> G | No |
| 445 | A -> G | No |
| 497 | A -> G | No |
| 508 | A -> G | No |
| 552 | G -> A | No |
| 561 | A -> G | No |
| 581 | T -> C | No |
| 604 | A -> G | No |
| 712 | T -> C | No |
| 728 | A -> G | No |
| 760 | C -> A | No |
| 780 | A -> G | No |
| 825 | A -> G | No |
| 857 | A -> G | No |
| 922 | T -> C | No |
| 935 | A -> G | No |
| 958 | A -> | No |
| 976 | T -> C | No |
| 1000 | A -> G | No |
| 1059 | C -> T | No |

Variant protein HSHGFR_P11 (SEQ ID NO:166) according to the present invention has an amino acid sequence as given at the end of the application; it is encoded by transcript(s) HSHGFR_T13 (SEQ ID NO:149). An alignment is given to the known protein (Hepatocyte growth factor precursor (SEQ ID NO:164)) at the end of the application. One or more alignments to one or more previously published protein sequences are given at the end of the application. A brief description of the relationship of the variant protein according to the present invention to each such aligned protein is as follows:

Comparison report between HSHGFR_P11 (SEQ ID NO:166) and HGF_HUMAN (SEQ ID NO:164):

1. An isolated chimeric polypeptide encoding for HSHGFR_P11 (SEQ ID NO:166), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEH corresponding to amino acids 1-160 of HGF_HUMAN (SEQ ID NO:164), which also corresponds to amino acids 1-160 of HSHGFR_P11 (SEQ ID NO:166), a second amino acid sequence being at least 90% homologous to SYRGKDLQENYCRNPRGEEGGPWCFTSNPEVRYEVCDIPQCSE corresponding to amino acids 166-208 of HGF_HUMAN (SEQ ID NO:164), which also corresponds to amino acids 161-203 of HSHGFR_P11 (SEQ ID NO:166), and a third amino acid sequence being at least 70%, optionally at least 80%, preferably at least 85%, more preferably at least 90% and most preferably at least 95% homologous to a polypeptide having the sequence GK corresponding to amino acids 204-205 of HSHGFR_P11 (SEQ ID NO:166), wherein said first amino acid sequence, second amino acid sequence and third amino acid sequence are contiguous and in a sequential order.

2. An isolated chimeric polypeptide encoding for an edge portion of HSHGFR_P11 (SEQ ID NO: 166), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HS, having a structure as follows: a sequence starting from any of amino acid numbers 160−x to 160; and ending at any of amino acid numbers 161+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSHGFR_P11 (SEQ ID NO:166) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HSHGFR_P11 (SEQ ID NO:166) Sequence provides support for the deduced sequence of this variant protein according to the present invention).

TABLE 10
Amino acid mutations

| SNP position(s) on amino acid | | Previously |
|---|---|---|
| sequence | Alternative amino acid(s) | known SNP? |
| 53 | I -> V | No |
| 58 | K -> R | No |
| 73 | R -> G | No |
| 90 | D -> G | No |
| 94 | K -> E | No |
| 118 | L -> P | No |
| 126 | R -> G | No |
| 162 | Y -> C | No |

The glycosylation sites of variant protein HSHGFR_P11 (SEQ ID NO:166), as compared to the known protein Hepatocyte growth factor precursor (SEQ ID NO:164), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | |
|---|---|
| acid sequence | Present in variant protein? |
| 653 | no |
| 476 | no |
| 566 | no |
| 402 | no |
| 294 | no |

The phosphorylation sites of variant protein HSHGFR_P11 (SEQ ID NO:166), as compared to the known protein Hepatocyte growth factor precursor (SEQ ID NO:164), are described in Table 12 (given according to their position(s) on the amino acid sequence in the first column; the second column indicates whether the phosphorylation site is present in the variant protein; and the last column indicates whether the position is different on the variant protein).

TABLE 12
Phosphorylation site(s)

| Position(s) on known amino | | Position in |
|---|---|---|
| acid sequence | Present in variant protein? | variant protein? |
| 32 | yes | 32 |

Variant protein HSHGFR_P11 (SEQ ID NO:166) is encoded by the following transcript(s): HSHGFR_T13 (SEQ ID NO:149), for which the sequence(s) is/are given at the end of the application. The coding portion of transcript HSHGFR_T13 (SEQ ID NO:149) is shown in bold; this coding portion starts at position 229 and ends at position 843. The transcript also has the following SNPs as listed in Table 13 (given according to their position on the nucleotide sequence, with the alternative nucleic acid listed; the last column indicates whether the SNP is known or not; the presence of known SNPs in variant protein HSHGFR_P11 (SEQ ID NO:166) Sequence provides support for the deduced sequence of this variant protein according to the present invention).

TABLE 13
Nucleic acid SNPs

| SNP position on nucleotide | | Previously |
|---|---|---|
| sequence | Alternative nucleic acid | known SNP? |
| 218 | C -> | No |
| 219 | C -> T | No |
| 256 | C -> T | No |
| 385 | A -> G | No |
| 401 | A -> G | No |
| 445 | A -> G | No |
| 497 | A -> G | No |
| 508 | A -> G | No |
| 552 | G -> A | No |
| 561 | A -> G | No |
| 581 | T -> C | No |
| 604 | A -> G | No |
| 713 | A -> G | No |
| 745 | C -> A | No |
| 765 | A -> G | No |
| 810 | A -> G | No |
| 948 | A -> G | No |

Variant protein HSHGFR_P12 (SEQ ID NO:167) according to the present invention has an amino acid sequence as given at the end of the application; it is encoded by transcript(s) HSHGFR_T14 (SEQ ID NO:150). An alignment is given to the known protein (Hepatocyte growth factor precursor (SEQ ID NO:164)) at the end of the application. One or more alignments to one or more previously published protein sequences are given at the end of the application. A brief description of the relationship of the variant protein according to the present invention to each such aligned protein is as follows:

Comparison report between HSHGFR_P12 (SEQ ID NO:167) and HGF_HUMAN (SEQ ID NO: 164):

1. An isolated chimeric polypeptide encoding for HSHGFR_P12 (SEQ ID NO:167), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEH corresponding to amino acids 1-160 of HGF_HUMAN (SEQ ID NO:164) which also corresponds to amino acids 1-160 of HSHGFR_P12 (SEQ ID NO:167), and a second amino acid sequence being at least 70%, optionally at least 80%, preferably at least 85%, more preferably at least 90% and most preferably at least 95% homologous to a polypeptide having the sequence R corresponding to amino acids 161-161 of HSHGFR_P12 (SEQ ID NO:167), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSHGFR_P12 (SEQ ID NO:167) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HSHGFR_P12 (SEQ ID NO:167) Sequence provides support for the deduced sequence of this variant protein according to the present invention).

TABLE 14
Amino acid mutations

| SNP position(s) on amino acid | | Previously |
|---|---|---|
| sequence | Alternative amino acid(s) | known SNP? |
| 53 | I -> V | No |
| 58 | K -> R | No |
| 73 | R -> G | No |
| 90 | D -> G | No |
| 94 | K -> E | No |
| 118 | L -> P | No |
| 126 | R -> G | No |

The glycosylation sites of variant protein HSHGFR_P12 (SEQ ID NO:167), as compared to the known protein Hepatocyte growth factor precursor (SEQ ID NO:164), are described in Table 15 (given according to their position(s) on the amino acid sequence in the first column; the second column indicates whether the glycosylation site is present in the variant protein; and the last column indicates whether the position is different on the variant protein).

TABLE 15
Glycosylation site(s)

| Position(s) on known amino | |
|---|---|
| acid sequence | Present in variant protein? |
| 653 | no |
| 476 | no |
| 566 | no |
| 402 | no |
| 294 | no |

The phosphorylation sites of variant protein HSHGFR_P12 (SEQ ID NO:167), as compared to the known protein Hepatocyte growth factor precursor (SEQ ID NO:164), are described in Table 16 (given according to their position(s) on the amino acid sequence in the first column; the second column indicates whether the phosphorylation site is present in the variant protein; and the last column indicates whether the position is different on the variant protein).

TABLE 16
Phosphorylation site(s)

| Position(s) on known amino | | Position in |
|---|---|---|
| acid sequence | Present in variant protein? | variant protein? |
| 32 | yes | 32 |

Variant protein HSHGFR_P12 (SEQ ID NO:167) is encoded by the following transcript(s): HSHGFR_T14 (SEQ ID NO:150), for which the sequence(s) is/are given at the end of the application. The coding portion of transcript HSHGFR_T14 (SEQ ID NO:150) is shown in bold; this coding portion starts at position 229 and ends at position 711. The transcript also has the following SNPs as listed in Table 17 (given according to their position on the nucleotide sequence, with the alternative nucleic acid listed; the last column indicates whether the SNP is known or not; the presence of known SNPs in variant protein HSHGFR_P12 (SEQ ID NO:167) Sequence provides support for the deduced sequence of this variant protein according to the present invention).

TABLE 17
Nucleic acid SNPs

| SNP position on nucleotide | | Previously |
|---|---|---|
| sequence | Alternative nucleic acid | known SNP? |
| 218 | C -> | No |
| 219 | C -> T | No |
| 256 | C -> T | No |
| 385 | A -> G | No |
| 401 | A -> G | No |
| 445 | A -> G | No |
| 497 | A -> G | No |
| 508 | A -> G | No |
| 552 | G -> A | No |
| 561 | A -> G | No |
| 581 | T -> C | No |
| 604 | A -> G | No |

Variant protein HSHGFR_P13 (SEQ ID NO:168) according to the present invention has an amino acid sequence as given at the end of the application; it is encoded by transcript(s) HSHGFR_T1. An alignment is given to the known protein (Hepatocyte growth factor precursor (SEQ ID NO:164)) at the end of the application. One or more alignments to one or more previously published protein sequences are given at the end of the application. A brief description of the relationship of the variant protein according to the present invention to each such aligned protein is as follows:

Comparison report between HSHGFR_P13 (SEQ ID NO:168) and HGF_HUMAN (SEQ ID NO:164):

1. An isolated chimeric polypeptide encoding for HSHGFR_P13 (SEQ ID NO:168), comprising a first amino acid sequence being at least 90% homologous to MWVTKLLPALLLQHVLLHLLLLPIAIPYAEGQRKRRNTIHEFKKSAKTTLIKIDPALKIKT KKVNTADQCANRCTRNKGLPFTCKAFVFDKARKQCLWFPFNSMSSGVKKEFGHEFDL YENKDYIRNCIIGKGRSYKGTVSITKSGIKCQPWSSMIPHEHSFLPSSYRGKDLQENYCR NPRGEEGGPWCFTSNPEVRYEVCDIPQCSEVECMTCNGESYRGLMDHTESGKICQRWD HQTPHRHKFLPERYPDKGFDDNYCRNPDGQPRPWCYTLDPHTRWEYCAIK corresponding to amino acids 1-286 of HGF_HUMAN (SEQ ID NO:164), which also corresponds to amino acids 1-286 of HSHGFR_P13 (SEQ ID NO:168), and a second amino acid sequence being at least 70%, optionally at least 80%, preferably at least 85%, more preferably at least 90% and most preferably at least 95% homologous to a polypeptide having the sequence NMRDITWALN (SEQ ID NO:494) corresponding to amino acids 287-296 of HSHGFR_P13 (SEQ ID NO:168), wherein said first amino acid sequence and second amino acid sequence are contiguous and in a sequential order.

2. An isolated polypeptide encoding for a tail of HSHGFR_P13 (SEQ ID NO:168), comprising a polypeptide being at least 70%, optionally at least about 80%, preferably at least about 85%, more preferably at least about 90% and most preferably at least about 95% homologous to the sequence NMRDITWALN (SEQ ID NO:494) in HSHGFR_P13 (SEQ ID NO:168).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSHGFR_P13 (SEQ ID NO:168) also has the following non-silent SNPs (Single Nucleotide Polymorphisms) as listed in Table 18, (given according to their position(s) on the amino acid sequence, with the alternative amino acid(s) listed; the last column indicates whether the SNP is known or not; the presence of known SNPs in variant protein HSHGFR_P13 (SEQ ID NO:168) Sequence provides support for the deduced sequence of this variant protein according to the present invention).

TABLE 18
Amino acid mutations

| SNP position(s) on amino acid | | Previously |
|---|---|---|
| sequence | Alternative amino acid(s) | known SNP? |
| 53 | I -> V | No |
| 58 | K -> R | No |
| 73 | R -> G | No |
| 90 | D -> G | No |
| 94 | K -> E | No |
| 118 | L -> P | No |
| 126 | R -> G | No |
| 162 | F -> L | No |
| 167 | Y -> C | No |
| 210 | E -> G | No |
| 232 | C -> R | No |
| 236 | D -> G | No |
| 244 | K -> | No |
| 250 | Y -> H | No |
| 258 | N -> D | No |

The glycosylation sites of variant protein HSHGFR_P13 (SEQ ID NO:168), as compared to the known protein Hepatocyte growth factor precursor (SEQ ID NO:164), are described in Table 19 (given according to their position(s) on the amino acid sequence in the first column; the second column indicates whether the glycosylation site is present in the variant protein; and the last column indicates whether the position is different on the variant protein).

TABLE 19
Glycosylation site(s)

| Position(s) on known amino | |
|---|---|
| acid sequence | Present in variant protein? |
| 653 | no |
| 476 | no |
| 566 | no |
| 402 | no |
| 294 | no |

The phosphorylation sites of variant protein HSHGFR_P13 (SEQ ID NO:168), as compared to the known protein Hepatocyte growth factor precursor (SEQ ID NO:164), are described in Table 20 (given according to their position(s) on the amino acid sequence in the first column; the second column indicates whether the phosphorylation site is present in the variant protein; and the last column indicates whether the position is different on the variant protein).

TABLE 20
Phosphorylation site(s)

| Position(s) on known amino | Present in | |
|---|---|---|
| acid sequence | variant protein? | Position in variant protein? |
| 32 | yes | 32 |

Variant protein HSHGFR_P13 (SEQ ID NO:168) is encoded by the following transcript(s): HSHGFR_T1 (SEQ ID NO:146), for which the sequence(s) is/are given at the end of the application. The coding portion of transcript HSHGFR_T1 (SEQ ID NO:146) is shown in bold; this coding portion starts at position 229 and ends at position 1115. The transcript also has the following SNPs as listed in Table 21 (given according to their position on the nucleotide sequence, with the alternative nucleic acid listed; the last column indicates whether the SNP is known or not; the presence of known SNPs in variant protein HSHGFR_P13 (SEQ ID NO:168) sequence provides support for the deduced sequence of this variant protein according to the present invention).

TABLE 21
Nucleic acid SNPs

| SNP position on nucleotide | Alternative | |
|---|---|---|
| sequence | nucleic acid | Previously known SNP? |
| 218 | C -> | No |
| 219 | C -> T | No |
| 256 | C -> T | No |
| 385 | A -> G | No |
| 401 | A -> G | No |
| 445 | A -> G | No |
| 497 | A -> G | No |
| 508 | A -> G | No |
| 552 | G -> A | No |
| 561 | A -> G | No |
| 581 | T -> C | No |
| 604 | A -> G | No |
| 712 | T -> C | No |
| 728 | A -> G | No |
| 760 | C -> A | No |
| 780 | A -> G | No |
| 825 | A -> G | No |
| 857 | A -> G | No |
| 922 | T -> C | No |
| 935 | A -> G | No |
| 958 | A -> | No |
| 976 | T -> C | No |
| 1000 | A -> G | No |
| 1059 | C -> T | No |
| 1094 | A -> C | No |
| 1117 | G -> A | No |
| 1203 | C -> T | No |
| 1353 | A -> T | No |

As noted above, cluster HSHGFR features 13 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSHGFR_node_{—}2 (SEQ ID NO:151) according to the present invention is supported by 10 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148), HSHGFR_T13 (SEQ ID NO:149) and HSHGFR_T14 (SEQ ID NO:150). Table 22 below describes the starting and ending position of this segment on each transcript.

TABLE 22
Segment location on transcripts

| | Segment | Segment |
|---|---|---|
| Transcript name | starting position | ending position |
| HSHGFR_T1 (SEQ ID NO: 146) | 1 | 171 |
| HSHGFR_T6 (SEQ ID NO: 147) | 1 | 171 |
| HSHGFR_T8 (SEQ ID NO: 148) | 1 | 171 |
| HSHGFR_T13 (SEQ ID NO: 149) | 1 | 171 |
| HSHGFR_T14 (SEQ ID NO: 150) | 1 | 171 |

Segment cluster HSHGFR_node_{—}2 (SEQ ID NO:152) according to the present invention is supported by 25 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148), HSHGFR_T13 (SEQ ID NO:149) and HSHGFR_T14 (SEQ ID NO:150). Table 23 below describes the starting and ending position of this segment on each transcript.

TABLE 23
Segment location on transcripts

| | Segment | Segment |
|---|---|---|
| Transcript name | starting position | ending position |
| HSHGFR_T1 (SEQ ID NO: 146) | 172 | 316 |
| HSHGFR_T6 (SEQ ID NO: 147) | 172 | 316 |
| HSHGFR_T8 (SEQ ID NO: 148) | 172 | 316 |
| HSHGFR_T13 (SEQ ID NO: 149) | 172 | 316 |
| HSHGFR_T14 (SEQ ID NO: 150) | 172 | 316 |

Segment cluster HSHGFR_node_{—}6 (SEQ ID NO:153) according to the present invention is supported by 31 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148), HSHGFR_T13 (SEQ ID NO:149) and HSHGFR_T14 (SEQ ID NO:150). Table 24 below describes the starting and ending position of this segment on each transcript.

TABLE 24
Segment location on transcripts

| | Segment | Segment |
|---|---|---|
| Transcript name | starting position | ending position |
| HSHGFR_T1 (SEQ ID NO: 146) | 317 | 482 |
| HSHGFR_T6 (SEQ ID NO: 147) | 317 | 482 |
| HSHGFR_T8 (SEQ ID NO: 148) | 317 | 482 |
| HSHGFR_T13 (SEQ ID NO: 149) | 317 | 482 |
| HSHGFR_T14 (SEQ ID NO: 150) | 317 | 482 |

Segment cluster HSHGFR_node_{—}11 (SEQ ID NO:154) according to the present invention is supported by 4 libraries. The number of libraries was determined as previously described. This segment can be found in the following transcript(s): HSHGFR_T14 (SEQ ID NO:150). Table 25 below describes the starting and ending position of this segment on each transcript.

TABLE 25
Segment location on transcripts

| | Segment | Segment |
|---|---|---|
| Transcript name | starting position | ending position |
| HSHGFR_T14 (SEQ ID NO: 150) | 711 | 1221 |

Segment cluster HSHGFR_node_{—}15 (SEQ ID NO:155) according to the present invention is supported by 24 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148) and HSHGFR_T13 (SEQ ID NO:149). Table 26 below describes the starting and ending position of this segment on each transcript.

TABLE 26
Segment location on transcripts

| | Segment | Segment |
|---|---|---|
| Transcript name | starting position | ending position |
| HSHGFR_T1 (SEQ ID NO: 146) | 726 | 853 |
| HSHGFR_T6 (SEQ ID NO: 147) | 726 | 853 |
| HSHGFR_T8 (SEQ ID NO: 148) | 726 | 853 |
| HSHGFR_T13 (SEQ ID NO: 149) | 711 | 838 |

Segment cluster HSHGFR_node_{—}16 (SEQ ID NO:156) according to the present invention is supported by 15 libraries. The number of libraries was determined as previously described. This segment can be found in the following transcript(s): HSHGFR_T13 (SEQ ID NO:149). Table 27 below describes the starting and ending position of this segment on each transcript.

TABLE 27
Segment location on transcripts

| | Segment | Segment |
|---|---|---|
| Transcript name | starting position | ending position |
| HSHGFR_T13 (SEQ ID NO: 149) | 839 | 2068 |

Segment cluster HSHGFR_node_{—}18 (SEQ ID NO:157) according to the present invention is supported by 25 libraries. The number of libraries was determined as previously described. This segment can be found in the following transcript(s): HSHGFR_T1 (SEQ ID NO:146), HSHGFR_T6 (SEQ ID NO:147) and HSHGFR_T8 (SEQ ID NO:148). Table 28 below describes the starting and ending position of this segment on each transcript.

TABLE 28
Segment location on transcripts

| | Segment | Segment |
|---|---|---|
| Transcript name | starting position | ending position |
| HSHGFR_T1 (SEQ ID NO: 146) | 854 | 974 |
| HSHGFR_T6 (SEQ ID NO: 147) | 854 | 974 |
| HSHGFR_T8 (SEQ ID NO: 148) | 854 | 974 |

Segment cluster HSHGFR_node_{—}22 (SEQ ID NO:158) according to the present invention is supported by 12 libraries. The number of libraries was determined as previously described. This segment can be found in the following transcript(s): HSHGFR_T1 (SEQ ID NO:146). Table 29 below describes the starting and ending position of this segment on each transcript.

TABLE 29
Segment location on transcripts

| | Segment | Segment |
|---|---|---|
| Transcript name | starting position | ending position |
| HSHGFR_T1 (SEQ ID NO: 146) | 1094 | 1353 |

Segment cluster HSHGFR_node_{—}24 (SEQ ID NO:159) according to the present invention is supported by 4 libraries. The number of libraries was determined as previously described. This segment can be found in the following transcript(s): HSHGFR_T6 (SEQ ID NO:147) and HSHGFR_T8 (SEQ ID NO:148). Table 30 below describes the starting and ending position of this segment on each transcript.

TABLE 30
Segment location on transcripts

| | Segment | Segment |
|---|---|---|
| Transcript name | starting position | ending position |
| HSHGFR_T6 (SEQ ID NO: 147) | 1094 | 1286 |
| HSHGFR_T8 (SEQ ID NO: 148) | 1094 | 1367 |

According to an optional embodiment of the present invention, short segments related to the above cluster are also provided. These segments are up to about 120 bp in length, and so are included in a separate description.

Segment cluster HSHGFR_node_{—}8 (SEQ ID NO:160) according to the present invention is supported by 26 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148), HSHGFR_T13 (SEQ ID NO:149) and HSHGFR_T14 (SEQ ID NO:150). Table 31 below describes the starting and ending position of this segment on each transcript.

TABLE 31
Segment location on transcripts

| | Segment | Segment |
|---|---|---|
| Transcript name | starting position | ending position |
| HSHGFR_T1 (SEQ ID NO: 146) | 483 | 595 |
| HSHGFR_T6 (SEQ ID NO: 147) | 483 | 595 |
| HSHGFR_T8 (SEQ ID NO: 148) | 483 | 595 |
| HSHGFR_T13 (SEQ ID NO: 149) | 483 | 595 |
| HSHGFR_T14 (SEQ ID NO: 150) | 483 | 595 |

Segment cluster HSHGFR_node_{—}10 (SEQ ID NO:161) according to the present invention is supported by 26 libraries. The number of libraries was determined as previously described. This segment can be found in the following transcript(s): HSHGFR_T1 (SEQ ID NO:146), HSHGFR_T6 (SEQ ID NO:147), HSHGFR_T8 (SEQ ID NO:148), HSHGFR_T13 (SEQ ID NO:149) and HSHGFR_T14 (SEQ ID NO:150). Table 32 below describes the starting and ending position of this segment on each transcript.

TABLE 32
Segment location on transcripts

| | Segment | Segment |
|---|---|---|
| Transcript name | starting position | ending position |
| HSHGFR_T1 (SEQ ID NO: 146) | 596 | 710 |
| HSHGFR_T6 (SEQ ID NO: 147) | 596 | 710 |
| HSHGFR_T8 (SEQ ID NO: 148) | 596 | 710 |
| HSHGFR_T13 (SEQ ID NO: 149) | 596 | 710 |
| HSHGFR_T14 (SEQ ID NO: 150) | 596 | 710 |

Segment cluster HSHGFR_node_{—}14 (SEQ ID NO:162) according to the present invention can be found in the following transcript(s): HSHGFR_T1 (SEQ ID NO:146), HSHGFR_T6 (SEQ ID NO:147) and HSHGFR_T8 (SEQ ID NO:148). Table 33 below describes the starting and ending position of this segment on each transcript.

TABLE 33
Segment location on transcripts

| | Segment | Segment |
|---|---|---|
| Transcript name | starting position | ending position |
| HSHGFR_T1 (SEQ ID NO: 146) | 711 | 725 |
| HSHGFR_T6 (SEQ ID NO: 147) | 711 | 725 |
| HSHGFR_T8 (SEQ ID NO: 148) | 711 | 725 |

Segment cluster HSHGFR_node_{—}20 (SEQ ID NO:163) according to the present invention is supported by 25 libraries. The number of libraries was determined as previously described. This segment can be found in the following transcript(s): HSHGFR_T1 (SEQ ID NO:146), HSHGFR_T6 (SEQ ID NO:147) and HSHGFR_T8 (SEQ ID NO:148). Table 34 below describes the starting and ending position of this segment on each transcript.

TABLE 34
3Segment location on transcripts

| | Segment | Segment |
|---|---|---|
| Transcript name | starting position | ending position |
| HSHGFR_T1 (SEQ ID NO: 146) | 975 | 1093 |
| HSHGFR_T6 (SEQ ID NO: 147) | 975 | 1093 |
| HSHGFR_T8 (SEQ ID NO: 148) | 975 | 1093 |

Variant protein alignment to the previously known protein:

| Sequence name: HGF_HUMAN (SEQ ID NO: 164) | | | |
|---|---|---|---|
| Sequence documentation: | | | |
| Alignment of: HSHGFR_P6 (SEQ ID NO: 165) × HGF_HUMAN (SEQ ID NO: 164) .. | | | |
| Alignment segment 1/1: | | | |
| Quality: | 2989.00 | Escore: | 0 |
| Matching length: | 290 | Total length: | 290 |
| Matching Percent Similarity: | 100.00 | Matching Percent Identity: | 99.66 |
| Total Percent Similarity: | 100.00 | Total Percent Identity: | 99.66 |
| Gaps: | 0 | | |

| Alignment: | | |
|---|---|---|
| | . . . . . | |
| 1 | MWVTKLLPALLLQHVLLHLLLLPIAIPYAEGQRKRRNTIHEFKKSAKTTL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MWVTKLLPALLLQHVLLHLLLLPIAIPYAEGQRKRRNTIHEFKKSAKTTL | 50 |
| | . . . . . | |
| 51 | IKIDPALKIKTKKVNTADQCANRCTRNKGLPFTCKAFVFDKARKQCLWFP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | IKIDPALKIKTKKVNTADQCANRCTRNKGLPFTCKAFVFDKARKQCLWFP | 100 |
| | . . . . . | |
| 101 | FNSMSSGVKKEFGHEFDLYENKDYIRNCIIGKGRSYKGTVSITKSGIKCQ | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FNSMSSGVKKEFGHEFDLYENKDYIRNCIIGKGRSYKGTVSITKSGIKCQ | 150 |
| | . . . . . | |
| 151 | PWSSMIPHEHSFLPSSYRGKDLQENYCRNPRGEEGGPWCFTSNPEVRYEV | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | PWSSMIPHEHSFLPSSYRGKOLQENYCRNPRGEEGGPWCFTSNPEVRYEV | 200 |
| | . . . . . | |
| 201 | CDIPQCSEVECMTCNGESYRGLMDHTESGKICQRWDHQTPHRHKFLPERY | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | CDIPQCSEVECMTCNGESYRGLMDHTESGKICQRWDHQTPHRHKFLPERY | 250 |
| | . . . . | |
| 251 | PDKGFDDNYCRNPDGQPRPWCYTLDPHTRWEYCAIKTCAE | 290 |
| | \|\|\|\|\|\|\|\|\|\|\|\|\|\|\|\|\|\|\|\|\|\|\|\|\|\|\|\|\|\|\|\|\|\|\|\|\|\|\|: | |
| 251 | PDKGFDDNYCRNPDGQPRPWCYTLDPHTRWEYCAIKTCAD | 290 |

| Sequence name: HGF_HUMAN (SEQ ID NO: 164) | | | |
|---|---|---|---|
| Sequence documentation: | | | |
| Alignment of: HSHGFR_P11 (SEQ ID NO: 166) × HGF_HUMAN (SEQ ID NO: 164) .. | | | |
| Alignment segment 1/1: | | | |
| Quality: | 1957.00 | Escore: | 0 |
| Matching length: | 203 | Total length: | 208 |
| Matching Percent Similarity: | 100.00 | Matching Percent Identity: | 100.00 |
| Total Percent Similarity: | 97.60 | Total Percent Identity: | 97.60 |
| Gaps: | 1 | | |

| Alignment: | | |
|---|---|---|
| | . . . . . | |
| 1 | MWVTKLLPALLLQHVLLHLLLLPIAIPYAEGQRKRRNTIHEFKKSAKTTL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MWVTKLLPALLLQHVLLHLLLLPIAIPYAEGQRKRRNTIHEFKKSAKTTL | 50 |
| | . . . . . | |
| 51 | IKIDPALKIKTKKVNTADQCANRCTRNKGLPFTCKAFVFDKARKQCLWFP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | IKIDPALKIKTKKVNTADQCANRCTRNKGLPFTCKAFVFDKARKQCLWFP | 100 |
| | . . . . . | |
| 101 | FNSMSSGVKKEFGHEFDLYENKDYIRNCIIGKGRSYKGTVSITKSGIKCQ | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FNSMSSGVKKEFGHEFDLYENKDYIRNCIIGKGRSYKGTVSITKSGIKCQ | 150 |
| | . . . . . | |
| 151 | PWSSMIPHEH.....SYRGKDLQENYCRNPRGEEGGPWCFTSNPEVRYEV | 195 |
| | \|\|\|\|\|\|\|\|\|\| \|\|\|\|\|\|\|\|\|\|\|\|\|\|\|\|\|\|\|\|\|\|\|\|\|\|\|\|\|\|\|\|\|\|\| | |
| 151 | PWSSMIPHEHSFLPSSYRGKDLQENYCRNPRGEEGGPWCFTSNPEVRYEV | 200 |
| 196 | CDIPQCSE | 203 |
| | \|\|\|\|\|\|\|\| | |
| 201 | CDIPQCSE | 208 |

| Sequence name: HGF_HUMAN (SEQ ID NO: 164) | | | |
|---|---|---|---|
| Sequence documentation: | | | |
| Alignment of: HSHGFR_P12 (SEQ ID NO: 167) × HGF_HUMAN (SEQ ID NO: 164) .. | | | |
| Alignment segment 1/1: | | | |
| Quality: | 1600.00 | Escore: | 0 |
| Matching length: | 160 | Total length: | 160 |
| Matching Percent Similarity: | 100.00 | Matching Percent Identity: | 100.00 |
| Total Percent Similarity: | 100.00 | Total Percent Identity: | 100.00 |
| Gaps: | 0 | | |

| Alignment: | | |
|---|---|---|
| | . . . . . | |
| 1 | MWVTKLLPALLLQHVLLHLLLLPIAIPYAEGQRKRRNTIHEFKKSAKTTL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MWVTKLLPALLLQHVLLHLLLLPIAIPYAEGQRKRRNTIHEFKKSAKTTL | 50 |
| | . . . . . | |
| 51 | IKIDPALKIKTKKVNTADQCANRCTRNKGLPFTCKAFVFDKARKQCLWFP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | IKIDPALKIKTKKVNTADQCANRCTRNEGLPFTCKAFVFDKARKQCLWFP | 100 |
| | . . . . . | |
| 101 | FNSMSSGVKKEFGHEFDLYENKDYIRNCIIGKGRSYKGTVSITKSGIKCQ | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FNSMSSGVKKEFGHEFDLYENKDYIRNCIIGKGRSYKGTVSITKSGIKCQ | 150 |
| | . | |
| 151 | PWSSMIPHEH | 160 |
| | \|\|\|\|\|\|\|\|\|\| | |
| 151 | PWSSMIPHEH | 160 |

| Sequence name: HGF_HUMAN (SEQ ID NO: 164) | | | |
|---|---|---|---|
| Sequence documentation: | | | |
| Alignment of: HSHGFR_P13 (SEQ ID NO: 168) × HGF_HUMAN (SEQ ID NO: 164) .. | | | |
| Alignment segment 1/1: | | | |
| Quality: | 2960.00 | Escore: | 0 |
| Matching length: | 292 | Total length: | 292 |
| Matching Percent Similarity: | 98.63 | Matching Percent Identity: | 98.63 |
| Total Percent Similarity: | 98.63 | Total Percent Identity: | 98.63 |
| Gaps: | 0 | | |

| Alignment: | | |
|---|---|---|
| | . . . . . | |
| 1 | MWVTKLLPALLLQHVLLHLLLLPIAIPYAEGQRKRRNTIHEFKKSAKTTL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MWVTKLLPALLLQHVLLHLLLLPIAIPYAEGQRKRRNTIHEFKKSAKTTL | 50 |
| | . . . . . | |
| 51 | IKIDPALKIKTKKVNTADQCANRCTRNKGLPFTCKAFVFDKARKQCLWFP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | IKIDPALKIKTKKVNTADQCANRCTRNKGLPFTCKAFVFDKARKQCLWFP | 100 |
| | . . . . . | |
| 101 | FNSMSSGVKKEFGHEFDLYENKDYIRNCIIGKGRSYKGTVSITKSGIKCQ | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FNSMSSGVKKEFGHEFDLYENKDYIRNCIIGKGRSYKGTVSITKSGIKCQ | 150 |
| | . . . . . | |
| 151 | PWSSMIPHEHSFLPSSYRGKDLQENYCRNPRGEEGGPWCFTSNPEVRYEV | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | PWSSMIPHEHSFLPSSYRGKDLQENYCRNPRGEEGGPWCFTSNPEVRYEV | 200 |
| | . . . . . | |
| 201 | CDIPQCSEVECMTCNGESYRGLMDHTESGKICQRWDHQTPHRHKFLPERY | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | CDIPQCSEVECMTCNGESYRGLMDHTESGKICQRWDHQTPHRHKFLPERY | 250 |
| | . . . . | |
| 251 | PDKGFDDNYCRNPDGQPRPWCYTLDPHTRWEYCAIKNMRDIT | 292 |
| | \|\|\|\|\|\|\|\|\|\|\|\|\|\|\|\|\|\|\|\|\|\|\|\|\|\|\|\|\|\|\|\|\|\|\|\| \| \| | |
| 251 | PDKGFDDNYCRNPDGQPRPWCYTLDPHTRWEYCAIKTCADNT | 292 |

### Description for Cluster S56892

Cluster S56892 features 4 transcript(s) and 20 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| S56892_PEA_1_T3 | 169 |
| S56892_PEA_1_T9 | 170 |
| S56892_PEA_1_T10 | 171 |
| S56892_PEA_1_T13 | 172 |

TABLE 2
Segments of interest

| Segment Name | Sequence ID No. |
|---|---|
| S56892_PEA_1_node_0 | 173 |
| S56892_PEA_1_node_5 | 174 |
| S56892_PEA_1_node_10 | 175 |
| S56892_PEA_1_node_18 | 176 |
| S56892_PEA_1_node_21 | 177 |
| S56892_PEA_1_node_3 | 178 |
| S56892_PEA_1_node_4 | 179 |
| S56892_PEA_1_node_6 | 180 |
| S56892_PEA_1_node_7 | 181 |
| S56892_PEA_1_node_8 | 182 |
| S56892_PEA_1_node_9 | 183 |
| S56892_PEA_1_node_12 | 184 |
| S56892_PEA_1_node_13 | 185 |
| S56892_PEA_1_node_14 | 186 |
| S56892_PEA_1_node_16 | 187 |
| S56892_PEA_1_node_17 | 188 |
| S56892_PEA_1_node_19 | 189 |
| S56892_PEA_1_node_20 | 190 |
| S56892_PEA_1_node_22 | 191 |
| S56892_PEA_1_node_23 | 192 |

TABLE 3
Proteins of interest

| | Sequence ID | |
|---|---|---|
| Protein Name | No. | Corresponding Transcript(s) |
| S56892_PEA_1_P2 | 194 | S56892_PEA_1_T3 (SEQ ID |
| | | NO: 169) |
| S56892_PEA_1_P8 | 195 | S56892_PEA_1_T9 (SEQ ID |
| | | NO: 170) |
| S56892_PEA_1_P9 | 196 | S56892_PEA_1_T10 (SEQ |
| | | ID NO: 171) |
| S56892_PEA_1_P11 | 197 | S56892_PEA_1_T13 (SEQ |
| | | ID NO: 172) |

These sequences are variants of the known protein Interleukin-6 precursor (SEQ ID NO:193) (SwissProt accession identifier IL6_HUMAN (SEQ ID NO:193); known also according to the synonyms IL-6; B-cell stimulatory factor 2; BSF-2; Interferon beta-2; Hybridoma growth factor; CTL differentiation factor; CDF), referred to herein as the previously known protein.

Protein Interleukin-6 precursor (SEQ ID NO:193) is known or believed to have the following function(s): IL-6 is a cytokine with a wide variety of biological functions: it plays an essential role in the final differentiation of B-cells into Ig-secreting cells, it induces myeloma and plasmacytoma growth, it induces nerve cells differentiation and in hepatocytes it induces acute phase reactants. The sequence for protein Interleukin-6 precursor is given at the end of the application, as “Interleukin-6 precursor amino acid sequence” (SEQ ID NO:193). Known polymorphisms for this sequence are as shown in Table 4.

TABLE 4
Amino acid mutations for Known Protein

| SNP position(s) on amino | |
|---|---|
| acid sequence | Comment |
| 32 | P -> S. /FTId = VAR_013075. |
| 162 | D -> V. /FTId = VAR_013076. |
| 173 | A -> V: ALMOST NO LOSS OF |
| | ACTIVITY. |
| 185 | W -> R: NO LOSS OF ACTIVITY. |
| 204 | S -> P: 87% LOSS OF ACTIVITY. |
| 210 | R -> K, E, Q, T, A, P: LOSS OF |
| | ACTIVITY. |
| 212 | M -> T, N, S, R: LOSS OF ACTIVITY. |

Protein Interleukin-6 precursor (SEQ ID NO:193) localization is believed to be Secreted.

Serum levels of IL-6 were significantly higher in women with endometriosis than in controls (P<0.001), with highest levels seen in women with chocolate cysts (Wieser et al, J Soc Gynecol Investig. 2003 January; 10(1):32-6). Variants of this cluster are suitable as diagnostic markers for endometriosis.

The previously known protein also has the following indication(s) and/or potential therapeutic use(s): Chemotherapy-induced injury; Cancer, sarcoma, Kaposi's; Cancer, myeloma; Chemotherapy-induced injury, bone marrow, thrombocytopenia; Thrombocytopenia; Infection, HIV/AIDS; Chemotherapy-induced injury, bone marrow, neutropenia; Cancer, breast; Cancer, colorectal; Cancer, leukaemia, acute myelogenous; Cancer, melanoma; Myelodysplastic syndrome; Hepatic dysfunction. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Interleukin 1 antagonist; Interleukin 2 agonist; Interleukin 6 modulator. A therapeutic role for a protein represented by the cluster has been predicted. The cluster was assigned this field because there was information in the drug database or the public databases (e.g., described herein above) that this protein, or part thereof, is used or can be used for a potential therapeutic indication: Antiarthritic, immunological; Radio/chemoprotective; Anticancer; Cytokine; Haematological; Anti-inflammatory; Antianaemic; Antiviral, interferon; Anabolic; Hepatoprotective.

The following GO Annotation(s) apply to the previously known protein. The following annotation(s) were found: skeletal development; acute-phase response; humoral defense mechanism; cell surface receptor linked signal transduction; cell-cell signaling; developmental processes; cell proliferation; positive control of cell proliferation; negative control of cell proliferation, which are annotation(s) related to Biological Process; cytokine; interleukin-6 receptor ligand, which are annotation(s) related to Molecular Function; and extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster S56892 features 4 transcript(s), which were listed in Table 1 above. These transcript(s) encode for protein(s) which are variant(s) of protein Interleukin-6 precursor (SEQ ID NO:193). A description of each variant protein according to the present invention is now provided.

Variant protein S56892_PEA_{—}1_P2 (SEQ ID NO:194) according to the present invention has an amino acid sequence as given at the end of the application; it is encoded by transcript(s) S56892_PEA_{—}1_T3 (SEQ ID NO:169). An alignment is given to the known protein (Interleukin-6 precursor (SEQ ID NO:193)) at the end of the application. One or more alignments to one or more previously published protein sequences are given at the end of the application. A brief description of the relationship of the variant protein according to the present invention to each such aligned protein is as follows:

Comparison report between S56892_PEA_{—}1_P2 (SEQ ID NO:194) and IL6_HUMAN (SEQ ID NO:193):

1. An isolated chimeric polypeptide encoding for S56892_PEA_{—}1_P2 (SEQ ID NO:194), comprising a first amino acid sequence being at least 70%, optionally at least 80%, preferably at least 85%, more preferably at least 90% and most preferably at least 95% homologous to a polypeptide having the sequence MNSFSTSKCRKSLALELPAAVEPCVREGCVAQGGLAGGQQQRQAPSCAVSSPLRSLPS GTG (SEQ ID NO:491) corresponding to amino acids 1-61 of S56892_PEA_{—}1_P2 (SEQ ID NO:194), and a second amino acid sequence being at least 90% homologous to AFGPVAFSLGLLLVLPAAFPAPVPPGEDSKDVAAPHRQPLTSSERIDKQIRYILDGISALR KETCNKSNMCESSKEALAENNLNLPKMAEKDGCFQSGFNEETCLVKIITGLLEFEVYLE YLQNRFESSEEQARAVQMSTKVLIQFLQKKAKNLDAITTPDPTTNASLLTKLQAQNQW LQDMTTHLILRSFKEFLQSSLRALRQM corresponding to amino acids 8-212 of IL6_HUMAN (SEQ ID NO:193), which also corresponds to amino acids 62-266 of S56892_PEA_{—}1_P2 (SEQ ID NO:194), wherein said first amino acid sequence and second amino acid sequence are contiguous and in a sequential order.

2. An isolated polypeptide encoding for a head of S56892_PEA_{—}1_P2 (SEQ ID NO:194), comprising a polypeptide being at least 70%, optionally at least about 80%, preferably at least about 85%, more preferably at least about 90% and most preferably at least about 95% homologous to the sequence MNSFSTSKCRKSLALELPAAVEPCVREGCVAQGGLAGGQQQRQAPSCAVSSPLRSLPS GTG (SEQ ID NO:491) of S56892_PEA_{—}1_P2 (SEQ ID NO:194).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because only one of the two trans-membrane region prediction programs (Tmpred: 1, Tmhmm: 0) Has predicted that this protein has a trans-membrane region. In addition both signal-peptide prediction programs predict that this protein is a non-secreted protein.

Variant protein S56892_PEA_{—}1_P2 (SEQ ID NO:194) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein S56892_PEA_{—}1_P2 (SEQ ID NO:194) Sequence provides support for the deduced sequence of this variant protein according to the present invention).

TABLE 5
Amino acid mutations

| SNP position(s) | | |
|---|---|---|
| on amino acid | Alternative | Previously |
| sequence | amino acid(s) | known SNP? |
| 224 | T -> | No |
| 231 | T -> A | No |
| 251 | S -> | No |

The glycosylation sites of variant protein S56892_PEA_{—}1_P2 (SEQ ID NO:194), as compared to the known protein Interleukin-6 precursor (SEQ ID NO:193), are described in Table 6 (given according to their position(s) on the amino acid sequence in the first column; the second column indicates whether the glycosylation site is present in the variant protein; and the last column indicates whether the position is different on the variant protein).

TABLE 6
Glycosylation site(s)

| Position(s) on known amino | Present in | Position in |
|---|---|---|
| acid sequence | variant protein? | variant protein? |
| 73 | yes | 127 |

Variant protein S56892_PEA_{—}1_P2 (SEQ ID NO:194) is encoded by the following transcript(s): S56892_PEA_{—}1_T3 (SEQ ID NO:169), for which the sequence(s) is/are given at the end of the application. The coding portion of transcript S56892_PEA_{—}1_T3 (SEQ ID NO:169) is shown in bold; this coding portion starts at position 458 and ends at position 1255. The transcript also has the following SNPs as listed in Table 7 (given according to their position on the nucleotide sequence, with the alternative nucleic acid listed; the last column indicates whether the SNP is known or not; the presence of known SNPs in variant protein S56892_PEA_{—}1_P2 (SEQ ID NO:194) Sequence provides support for the deduced sequence of this variant protein according to the present invention).

TABLE 7
Nucleic acid SNPs

| SNP position on nucleotide | Alternative | Previously |
|---|---|---|
| sequence | nucleic acid | known SNP? |
| 407 | A -> T | No |
| 408 | G -> T | No |
| 706 | A -> G | No |
| 1128 | C -> | No |
| 1148 | A -> G | No |
| 1209 | G -> | No |
| 1222 | C -> T | No |
| 1594 | -> A | No |
| 1594 | -> T | No |

Variant protein S56892-PEA_{—}1_P8 (SEQ ID NO:195) according to the present invention has an amino acid sequence as given at the end of the application; it is encoded by transcript(s) S56892_PEA_{—}1_T9 (SEQ ID NO:170). An alignment is given to the known protein (Interleukin-6 precursor (SEQ ID NO:193)) at the end of the application. One or more alignments to one or more previously published protein sequences are given at the end of the application. A brief description of the relationship of the variant protein according to the present invention to each such aligned protein is as follows:

Comparison report between S56892_PEA_{—}1_P8 (SEQ ID NO:195) and IL6_HUMAN (SEQ ID NO:193):

1. An isolated chimeric polypeptide encoding for S56892_PEA_{—}1_P8 (SEQ ID NO:195), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSNMCESSKEALAENNLNLPKMAEKDGCFQSGFNEETCLVKIITGLL EFEVYLEYLQNRFESSEEQARAVQMSTKVLIQFLQKK corresponding to amino acids 1-157 of IL6_HUMAN (SEQ ID NO:193), which also corresponds to amino acids 1-157 of S56892_PEA_{—}1_P8 (SEQ ID NO:195), and a second amino acid sequence being at least 70%, optionally at least 80%, preferably at least 85%, more preferably at least 90% and most preferably at least 95% homologous to a polypeptide having the sequence VGVSSFPQLGVGEDRLKDSVLDNSGMQCHFQKRRLHVNKRV (SEQ ID NO:492) corresponding to amino acids 158-198 of S56892_PEA_{—}1_P8 (SEQ ID NO:195), wherein said first amino acid sequence and second amino acid sequence are contiguous and in a sequential order.

2. An isolated polypeptide encoding for a tail of S56892_PEA_{—}1_P8 (SEQ ID NO:195), comprising a polypeptide being at least 70%, optionally at least about 80%, preferably at least about 85%, more preferably at least about 90% and most preferably at least about 95% homologous to the sequence VGVSSFPQLGVGEDRLKDSVLDNSGMQCHFQKRRLHVNKRV (SEQ ID NO:492) in S56892_PEA_{—}1_P8 (SEQ ID NO:195).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein S56892_PEA_{—}1_P8 (SEQ ID NO:195), as compared to the known protein Interleukin-6 precursor (SEQ ID NO:193), are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on known amino | Present in | Position in |
|---|---|---|
| acid sequence | variant protien? | variant protein? |
| 73 | yes | 73 |

Variant protein S56892_PEA_{—}1_P8 (SEQ ID NO:195) is encoded by the following transcript(s): S56892_PEA_{—}1_T9 (SEQ ID NO:170), for which the sequence(s) is/are given at the end of the application. The coding portion of transcript S56892_PEA_{—}1_T9 (SEQ ID NO:170) is shown in bold; this coding portion starts at position 458 and ends at position 1051. The transcript also has the following SNPs as listed in Table 9 (given according to their position on the nucleotide sequence, with the alternative nucleic acid listed; the last column indicates whether the SNP is known or not; the presence of known SNPs in variant protein S56892_PEA_{—}1_P8 (SEQ ID NO:195) Sequence provides support for the deduced sequence of this variant protein according to the present invention).

TABLE 9
Nucleic acid SNPs

| SNP position on nucleotide | Alternative | Previously |
|---|---|---|
| sequence | nucleic acid | known SNP? |
| 407 | A -> T | No |
| 408 | G -> T | No |
| 544 | A -> G | No |
| 1798 | A -> G | Yes |
| 2257 | G -> A | Yes |
| 2711 | C -> | No |
| 2731 | A -> G | No |
| 2792 | G -> | No |
| 2805 | C -> T | No |
| 3177 | -> A | No |
| 3177 | -> T | No |

Variant protein S56892_PEA_{—}1_P9 (SEQ ID NO:196) according to the present invention has an amino acid sequence as given at the end of the application; it is encoded by transcript(s) S56892_PEA_{—}1_T10 (SEQ ID NO:171). An alignment is given to the known protein (Interleukin-6 precursor (SEQ ID NO:193)) at the end of the application. One or more alignments to one or more previously published protein sequences are given at the end of the application. A brief description of the relationship of the variant protein according to the present invention to each such aligned protein is as follows:

Comparison report between S56892_PEA_{—}1_P9 (SEQ ID NO:196) and IL6_HUMAN (SEQ ID NO:193):

1. An isolated chimeric polypeptide encoding for S56892_PEA_{—}1_P9 (SEQ ID NO:196), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSNMCESSKEALAENNLNLPKMAEKDGCFQSGFNE corresponding to amino acids 1-108 of IL6_HUMAN (SEQ ID NO:193), which also corresponds to amino acids 1-108 of S56892_PEA_{—}1_P9 (SEQ ID NO:196), and a second amino acid sequence being at least 90% homologous to AKNLDAITTPDPTTNASLLTKLQAQNQWLQDMTTHLILRSFKEFLQSSLRALRQM corresponding to amino acids 158-212 of IL6_HUMAN (SEQ ID NO:193), which also corresponds to amino acids 109-163 of S56892_PEA_{—}1_P9 (SEQ ID NO:196), wherein said first amino acid sequence and second amino acid sequence are contiguous and in a sequential order.

2. An isolated chimeric polypeptide encoding for an edge portion of S56892_PEA_{—}1_P9 (SEQ ID NO:196), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EA, having a structure as follows: a sequence starting from any of amino acid numbers 108−x to 108; and ending at any of amino acid numbers 109+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S56892_PEA_{—}1_P9 (SEQ ID NO:196) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein S56892_PEA_{—}1_P9 (SEQ ID NO:196) Sequence provides support for the deduced sequence of this variant protein according to the present invention).

TABLE 10
Amino acid mutations

| SNP position(s) on amino acid | Alternative amino | Previously |
|---|---|---|
| sequence | acid(s) | known SNP? |
| 121 | T -> | No |
| 128 | T -> A | No |
| 148 | S -> | No |

The glycosylation sites of variant protein S56892_PEA_{—}1_P9 (SEQ ID NO:196), as compared to the known protein Interleukin-6 precursor (SEQ ID NO:193), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | Present in | Position in |
|---|---|---|
| acid sequence | variant protein? | variant protein? |
| 73 | yes | 73 |

Variant protein S56892_PEA_{—}1_P9 (SEQ ID NO:196) is encoded by the following transcript(s): S56892_PEA_{—}1_T10 (SEQ ID NO:171), for which the sequence(s) is/are given at the end of the application. The coding portion of transcript S56892_PEA_{—}1_T10 (SEQ ID NO:171) is shown in bold; this coding portion starts at position 113 and ends at position 601. The transcript also has the following SNPs as listed in Table 12 (given according to their position on the nucleotide sequence, with the alternative nucleic acid listed; the last column indicates whether the SNP is known or not; the presence of known SNPs in variant protein S56892_PEA_{—}1_P9 (SEQ ID NO:196) Sequence provides support for the deduced sequence of this variant protein according to the present invention).

TABLE 12
Nucleic acid SNPs

| SNP position on nucleotide | Alternative | Previously |
|---|---|---|
| sequence | nucleic acid | known SNP? |
| 62 | A -> T | No |
| 63 | G -> T | No |
| 199 | A -> G | No |
| 474 | C -> | No |
| 494 | A -> G | No |
| 555 | G -> | No |
| 568 | C -> T | No |
| 940 | -> A | No |
| 940 | -> T | No |

Variant protein S56892_PEA_{—}1_P11 (SEQ ID NO:197) according to the present invention has an amino acid sequence as given at the end of the application; it is encoded by transcript(s) S56892_PEA_{—}1_T13 (SEQ ID NO:172). An alignment is given to the known protein (Interleukin-6 precursor (SEQ ID NO:193)) at the end of the application. One or more alignments to one or more previously published protein sequences are given at the end of the application. A brief description of the relationship of the variant protein according to the present invention to each such aligned protein is as follows:

Comparison report between S56892_PEA_{—}1_P11 (SEQ ID NO:197) and IL6_HUMAN (SEQ ID NO:193):

1. An isolated chimeric polypeptide encoding for S56892_PEA_{—}1_P11 (SEQ ID NO:197), comprising a first amino acid sequence being at least 90% homologous to MNSFSTSAFGPVAFSLGLLLVLPAAFPAPVPPGEDSKDVAAPHRQPLTSSERIDKQIRYIL DGISALRKETCNKSN corresponding to amino acids 1-76 of IL6_HUMAN (SEQ ID NO:193), which also corresponds to amino acids 1-76 of S56892_PEA_{—}1_P11 (SEQ ID NO:197), and a second amino acid sequence being at least 70%, optionally at least 80%, preferably at least 85%, more preferably at least 90% and most preferably at least 95% homologous to a polypeptide having the sequence IWLKKMDASNLDSMRRLAW (SEQ ID NO:493) corresponding to amino acids 77-95 of S56892_PEA_{—}1_P11 (SEQ ID NO:197), wherein said first amino acid sequence and second amino acid sequence are contiguous and in a sequential order.

2. An isolated polypeptide encoding for a tail of S56892_PEA_{—}1_P11 (SEQ ID NO:197), comprising a polypeptide being at least 70%, optionally at least about 80%, preferably at least about 85%, more preferably at least about 90% and most preferably at least about 95% homologous to the sequence IWLKKMDASNLDSMRRLAW (SEQ ID NO:493) in S56892_PEA_{—}1_P11 (SEQ ID NO:197).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein S56892_PEA_{—}1_P11 (SEQ ID NO:197), as compared to the known protein Interleukin-6 precursor (SEQ ID NO:193), are described in Table 13 (given according to their position(s) on the amino acid sequence in the first column; the second column indicates whether the glycosylation site is present in the variant protein; and the last column indicates whether the position is different on the variant protein).

TABLE 13
Glycosylation site(s)

| Position(s) on known amino | Present in | Position in |
|---|---|---|
| acid sequence | variant protein? | variant protein? |
| 73 | yes | 73 |

Variant protein S56892_PEA_{—}1_P11 (SEQ ID NO:197) is encoded by the following transcript(s): S56892_PEA_{—}1_T13 (SEQ ID NO:172), for which the sequence(s) is/are given at the end of the application. The coding portion of transcript S56892_PEA_{—}1_T13 (SEQ ID NO:172) is shown in bold; this coding portion starts at position 458 and ends at position 742. The transcript also has the following SNPs as listed in Table 14 (given according to their position on the nucleotide sequence, with the alternative nucleic acid listed; the last column indicates whether the SNP is known or not; the presence of known SNPs in variant protein S56892_PEA_{—}1_P11 (SEQ ID NO:197) Sequence provides support for the deduced sequence of this variant protein according to the present invention).

TABLE 14
Nucleic acid SNPs

| SNP position on nucleotide | Alternative | Previously |
|---|---|---|
| sequence | nucleic acid | known SNP? |
| 407 | A -> T | No |
| 408 | G -> T | No |
| 544 | A -> G | No |
| 914 | C -> | No |
| 934 | A -> G | No |
| 995 | G -> | No |
| 1008 | C -> T | No |
| 1380 | -> A | No |
| 1380 | -> T | No |

As noted above, cluster S56892 features 20 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster S56892_PEA_{—}1_node_{—}0 (SEQ ID NO:173) according to the present invention is supported by 2 libraries. The number of libraries was determined as previously described. This segment can be found in the following transcript(s): S56892_PEA_{—}1_T3 (SEQ ID NO:169), S56892_PEA_{—}1_T9 (SEQ ID NO:170) and S56892_PEA_{—}1_T13 (SEQ ID NO:172). Table 15 below describes the starting and ending position of this segment on each transcript.

TABLE 15
Segment location on transcripts

| | Segment | Segment |
|---|---|---|
| | starting | ending |
| Transcript name | position | position |
| S56892_PEA_1_T3 (SEQ ID NO: 169) | 1 | 373 |
| S56892_PEA_1_T9 (SEQ ID NO: 170) | 1 | 373 |
| S56892_PEA_1_T13 (SEQ ID | 1 | 373 |
| NO: 172) | | |

Segment cluster S56892_PEA_{—}1_node_{—}5 (SEQ ID NO:174) according to the present invention is supported by 6 libraries. The number of libraries was determined as previously described. This segment can be found in the following transcript(s): S56892_PEA_{—}1_T3 (SEQ ID NO:169). Table 16 below describes the starting and ending position of this segment on each transcript.

TABLE 16
Segment location on transcripts

| | Segment | Segment |
|---|---|---|
| | starting | ending |
| Transcript name | position | position |
| S56892_PEA_1_T3 (SEQ ID NO: 169) | 477 | 632 |

Segment cluster S56892_PEA_{—}1_node_{—}10 (SEQ ID NO:175) according to the present invention is supported by 98 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 17 below describes the starting and ending position of this segment on each transcript.

TABLE 17
Segment location on transcripts

| | Segment | Segment |
|---|---|---|
| | starting | ending |
| Transcript name | position | position |
| S56892_PEA_1_T3 (SEQ ID NO: 169) | 708 | 829 |
| S56892_PEA_1_T9 (SEQ ID NO: 170) | 546 | 667 |
| S56892_PEA_1_T10 (SEQ ID | 201 | 322 |
| NO: 171) | | |
| S56892_PEA_1_T13 (SEQ ID | 546 | 667 |
| NO: 172) | | |

Segment cluster S56892_PEA_{—}1_node_{—}18 (SEQ ID NO:176) according to the present invention is supported by 22 libraries. The number of libraries was determined as previously described. This segment can be found in the following transcript(s): S56892_PEA_{—}1_T9 (SEQ ID NO:170). Table 18 below describes the starting and ending position of this segment on each transcript.

TABLE 18
Segment location on transcripts

| | Segment | Segment |
|---|---|---|
| | starting | ending |
| Transcript name | position | position |
| S56892_PEA_1_T9 (SEQ ID NO: 170) | 929 | 2673 |

Segment cluster S56892_PEA_{—}1_node_{—}21 (SEQ ID NO:177) according to the present invention is supported by 111 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 19 below describes the starting and ending position of this segment on each transcript.

TABLE 19
Segment location on transcripts

| | Segment | Segment |
|---|---|---|
| | starting | ending |
| Transcript name | position | position |
| S56892_PEA_1_T3 (SEQ ID NO: 169) | 1169 | 1625 |
| S56892_PEA_1_T9 (SEQ ID NO: 170) | 2752 | 3208 |
| S56892_PEA_1_T10 (SEQ ID | 515 | 971 |
| NO: 171) | | |
| S56892_PEA_1_T13 (SEQ ID | 955 | 1411 |
| NO: 172) | | |

According to an optional embodiment of the present invention, short segments related to the above cluster are also provided. These segments are up to about 120 bp in length, and so are included in a separate description.

Segment cluster S56892_PEA_{—}1_node_{—}3 (SEQ ID NO:178) according to the present invention is supported by 1 libraries. The number of libraries was determined as previously described. This segment can be found in the following transcript(s): S56892_PEA_{—}1_T10 (SEQ ID NO:171). Table 20 below describes the starting and ending position of this segment on each transcript.

TABLE 20
Segment location on transcripts

| | Segment | Segment |
|---|---|---|
| Transcript name | starting position | ending position |
| S56892_PEA_1_T10 (SEQ ID | 1 | 28 |
| NO: 171) | | |

Segment cluster S56892_PEA_{—}1_node_{—}4 (SEQ ID NO:179) according to the present invention is supported by 93 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 21 below describes the starting and ending position of this segment on each transcript.

TABLE 21
Segment location on transcripts

| | Segment | Segment |
|---|---|---|
| | starting | ending |
| Transcript name | position | position |
| S56892_PEA_1_T3 (SEQ ID NO: 169) | 374 | 476 |
| S56892_PEA_1_T9 (SEQ ID NO: 170) | 374 | 476 |
| S56892_PEA_1_T10 (SEQ ID | 29 | 131 |
| NO: 171) | | |
| S56892_PEA_1_T13 (SEQ ID | 374 | 476 |
| NO: 172) | | |

Segment cluster S56892_PEA_{—}1_node_{—}6 (SEQ ID NO:180) according to the present invention can be found in the following transcript(s): S56892_PEA_{—}1_T3 (SEQ ID NO:169). Table 22 below describes the starting and ending position of this segment on each transcript.

TABLE 22
Segment location on transcripts

| | Segment | Segment |
|---|---|---|
| | starting | ending |
| Transcript name | position | position |
| S56892_PEA_1_T3 (SEQ ID NO: 169) | 633 | 638 |

Segment cluster S56892_PEA_{—}1_node_{—}7 (SEQ ID NO:181) according to the present invention can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 23 below describes the starting and ending position of this segment on each transcript.

TABLE 23
Segment location on transcripts

| | Segment | Segment |
|---|---|---|
| | starting | ending |
| Transcript name | position | position |
| S56892_PEA_1_T3 (SEQ ID NO: 169) | 639 | 657 |
| S56892_PEA_1_T9 (SEQ ID NO: 170) | 477 | 495 |
| S56892_PEA_1_T10 (SEQ ID | 132 | 150 |
| NO: 171) | | |
| S56892_PEA_1_T13 (SEQ ID | 477 | 495 |
| NO: 172) | | |

Segment cluster S56892_PEA_{—}1_node_{—}8 (SEQ ID NO:182) according to the present invention is supported by 89 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 24 below describes the starting and ending position of this segment on each transcript.

TABLE 24
Segment location on transcripts

| | Segment | Segment |
|---|---|---|
| | starting | ending |
| Transcript name | position | position |
| S56892_PEA_1_T3 (SEQ ID NO: 169) | 658 | 693 |
| S56892_PEA_1_T9 (SEQ ID NO: 170) | 496 | 531 |
| S56892_PEA_1_T10 (SEQ ID | 151 | 186 |
| NO: 171) | | |
| S56892_PEA_1_T13 (SEQ ID | 496 | 531 |
| NO: 172) | | |

Segment cluster S56892_PEA_{—}1_node_{—}9 (SEQ ID NO:183) according to the present invention can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 25 below describes the starting and ending position of this segment on each transcript.

TABLE 25
Segment location on transcripts

| | Segment | Segment |
|---|---|---|
| | starting | ending |
| Transcript name | position | position |
| S56892_PEA_1_T3 (SEQ ID NO: 169) | 694 | 707 |
| S56892_PEA_1_T9 (SEQ ID NO: 170) | 532 | 545 |
| S56892_PEA_1_T10 (SEQ ID | 187 | 200 |
| NO: 171) | | |
| S56892_PEA_1_T13 (SEQ ID | 532 | 545 |
| NO: 172) | | |

Segment cluster S56892_PEA_{—}1_node_{—}12 (SEQ ID NO:184) according to the present invention can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 26 below describes the starting and ending position of this segment on each transcript.

TABLE 26
Segment location on transcripts

| | Segment | |
|---|---|---|
| | starting | Segment |
| Transcript name | position | ending position |
| S56892_PEA_1_T3 (SEQ ID NO: 169) | 830 | 849 |
| S56892_PEA_1_T9 (SEQ ID NO: 170) | 668 | 687 |
| S56892_PEA_1_T10 (SEQ ID | 323 | 342 |
| NO: 171) | | |
| S56892_PEA_1_T13 (SEQ ID | 668 | 687 |
| NO: 172) | | |

Segment cluster S56892_PEA_{—}1_node_{—}13 (SEQ ID NO:185) according to the present invention is supported by 70 libraries. The number of libraries was determined as previously described. This segment can be found in the following transcript(s): S56892_PEA_{—}1_T3 (SEQ ID NO:169), S56892_PEA_{—}1_T9 (SEQ ID NO:170) and S56892_PEA_{—}1_T10 (SEQ ID NO:171). Table 27 below describes the starting and ending position of this segment on each transcript.

TABLE 27
Segment location on transcripts

| | Segment | |
|---|---|---|
| | starting | Segment |
| Transcript name | position | ending position |
| S56892_PEA_1_T3 (SEQ ID NO: 169) | 850 | 901 |
| S56892_PEA_1_T9 (SEQ ID NO: 170) | 688 | 739 |
| S56892_PEA_1_T10 (SEQ ID | 343 | 394 |
| NO: 171) | | |

Segment cluster S56892_PEA_{—}1_node_{—}14 (SEQ ID NO:186) according to the present invention is supported by 64 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 28 below describes the starting and ending position of this segment on each transcript.

TABLE 28
Segment location on transcripts

| | Segment | |
|---|---|---|
| | starting | Segment |
| Transcript name | position | ending position |
| S56892_PEA_1_T3 (SEQ ID NO: 169) | 902 | 943 |
| S56892_PEA_1_T9 (SEQ ID NO: 170) | 740 | 781 |
| S56892_PEA_1_T10 (SEQ ID | 395 | 436 |
| NO: 171) | | |
| S56892_PEA_1_T13 (SEQ ID | 688 | 729 |
| NO: 172) | | |

Segment cluster S56892_PEA_{—}1_node_{—}16 (SEQ ID NO:187) according to the present invention is supported by 78 libraries. The number of libraries was determined as previously described. This segment can be found in the following transcript(s): S56892_PEA_{—}1_T3 (SEQ ID NO:169), S56892_PEA_{—}1_T9 (SEQ ID NO:170) and S56892_PEA_{—}1_T13 (SEQ ID NO:172). Table 29 below describes the starting and ending position of this segment on each transcript.

TABLE 29
Segment location on transcripts

| | Segment | |
|---|---|---|
| | starting | Segment |
| Transcript name | position | ending position |
| S56892_PEA_1_T3 (SEQ ID NO: 169) | 944 | 1051 |
| S56892_PEA_1_T9 (SEQ ID NO: 170) | 782 | 889 |
| S56892_PEA_1_T13 (SEQ ID | 730 | 837 |
| NO: 172) | | |

Segment cluster S56892_PEA_{—}1_node_{—}17 (SEQ ID NO:188) according to the present invention is supported by 73 libraries. The number of libraries was determined as previously described. This segment can be found in the following transcript(s): S56892_PEA_{—}1_T3 (SEQ ID NO:169), S56892_PEA_{—}1_T9 (SEQ ID NO:170) and S56892_PEA_{—}1_T13 (SEQ ID NO:172). Table 30 below describes the starting and ending position of this segment on each transcript.

TABLE 30
Segment location on transcripts

| | Segment | |
|---|---|---|
| | starting | Segment |
| Transcript name | position | ending position |
| S56892_PEA_1_T3 (SEQ ID NO: 169) | 1052 | 1090 |
| S56892_PEA_1_T9 (SEQ ID NO: 170) | 890 | 928 |
| S56892_PEA_1_T13 (SEQ ID | 838 | 876 |
| NO: 172) | | |

Segment cluster S56892_PEA_{—}1_node_{—}19 (SEQ ID NO:189) according to the present invention is supported by 78 libraries. The number of libraries was determined as previously described. This segment can be found in the following transcript(s): S56892_PEA_{—}1_T3 (SEQ ID NO:169), S56892_PEA_{—}1_T9 (SEQ ID NO:170) S56892_PEA_{—}1_T10 (SEQ ID NO:171) and S56892_PEA_{—}1_T13 (SEQ ID NO:172). Table 31 below describes the starting and ending position of this segment on each transcript.

TABLE 31
Segment location on transcripts

| | Segment | |
|---|---|---|
| | starting | Segment |
| Transcript name | position | ending position |
| S56892_PEA_1_T3 (SEQ ID NO: 169) | 1091 | 1124 |
| S56892_PEA_1_T9 (SEQ ID NO: 170) | 2674 | 2707 |
| S56892_PEA_1_T10 (SEQ ID | 437 | 470 |
| NO: 171) | | |
| S56892_PEA_1_T13 (SEQ ID | 877 | 910 |
| NO: 172) | | |

Segment cluster S56892_PEA_{—}1_node_{—}20 (SEQ ID NO:190) according to the present invention is supported by 83 libraries. The number of libraries was determined as previously described. This segment can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 32 below describes the starting and ending position of this segment on each transcript.

TABLE 32
Segment location on transcripts

| | Segment | |
|---|---|---|
| | starting | Segment |
| Transcript name | position | ending position |
| S56892_PEA_1_T3 (SEQ ID NO: 169) | 1125 | 1168 |
| S56892_PEA_1_T9 (SEQ ID NO: 170) | 2708 | 2751 |
| S56892_PEA_1_T10 (SEQ ID | 471 | 514 |
| NO: 171) | | |
| S56892_PEA_1_T13 (SEQ ID | 911 | 954 |
| NO: 172) | | |

Segment cluster S56892_PEA_{—}1_node_{—}22 (SEQ ID NO:191) according to the present invention can be found in the following transcript(s): S56892_PEA_{—}1_T3 (SEQ ID NO:169), S56892_PEA_{—}1_T9 (SEQ ID NO:170), S56892_PEA_{—}1_T10 (SEQ ID NO:171) and S56892_PEA_{—}1_T13 (SEQ ID NO:172). Table 33 below describes the starting and ending position of this segment on each transcript.

TABLE 33
Segment location on transcripts

| | Segment | |
|---|---|---|
| | starting | Segment |
| Transcript name | position | ending position |
| S56892_PEA_1_T3 (SEQ ID NO: 169) | 1626 | 1638 |
| S56892_PEA_1_T9 (SEQ ID NO: 170) | 3209 | 3221 |
| S56892_PEA_1_T10 (SEQ ID | 972 | 984 |
| NO: 171) | | |
| S56892_PEA_1_T13 (SEQ ID | 1412 | 1424 |
| NO: 172) | | |

Segment cluster S56892_PEA_{—}1_node_{—}23 (SEQ ID NO:192) according to the present invention is supported by 58 libraries. The number of libraries was determined as previously described. This segment can be found in the following transcript(s): S56892_PEA_{—}1_T3 (SEQ ID NO:169, S56892_PEA_{—}1_T9 (SEQ ID NO:170), S56892_PEA_{—}1_T10 (SEQ ID NO:171) and S56892_PEA_{—}1_T13 (SEQ ID NO:172). Table 34 below describes the starting and ending position of this segment on each transcript.

TABLE 34
Segment location on transcripts

| | Segment | |
|---|---|---|
| | starting | Segment |
| Transcript name | position | ending position |
| S56892_PEA_1_T3 (SEQ ID NO: 169) | 1639 | 1696 |
| S56892_PEA_1_T9 (SEQ ID NO: 170) | 3222 | 3279 |
| S56892_PEA_1_T10 (SEQ ID | 985 | 1042 |
| NO: 171) | | |
| S56892_PEA_1_T13 (SEQ ID | 1425 | 1482 |
| NO: 172) | | |

Variant protein alignment to the previously known protein:

| Sequence name: IL6_HUMAN (SEQ ID NO:193) | | | |
|---|---|---|---|
| Sequence documentation: | | | |
| Alignment of: S56892_PEA_1_P2 (SEQ ID NO:194) × | | | |
| IL6_HUMAN (SEQ ID NO:193) . . | | | |
| Alignment segment 1/1: | | | |
| Quality: | 1997.00 | Escore: | 0 |
| Matching length: | 207 | Total length: | 207 |
| Matching Percent | 99.52 | Matching Percent | 99.52 |
| Similarity: | | Identity: | |
| Total Percent | 99.52 | Total Percent | 99.52 |
| Similarity: | | Identity: | |
| Gaps: | 0 | | |

| Alignment: | | |
|---|---|---|
| | . . . . . | |
| 60 | TGAFGPVAFSLGLLLVLPAAFPAPVPPGEDSKDVAAPHRQPLTSSERIDK | 109 |
| | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 6 | TSAFGPVAFSLGLLLVLPAAFPAPVPPGEDSKDVAAPHRQPLTSSERIDK | 55 |
| | . . . . . | |
| 110 | QIRYILDGISALRKETCNKSNMCESSKEALAENNLNLPKMAEKDGCFQSG | 159 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 56 | QIRYILDGISALRKETCNKSNMCESSKEALAENNLNLPKMAEKDGCFQSG | 105 |
| | . . . . . | |
| 160 | FNEETCLVKIITGLLEFEVYLEYLQNRFESSEEQARAVQMSTKVLIQFLQ | 209 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 106 | FNEETCLVKIITGLLEFEVYLEYLQNRFESSEEQARAVQMSTKVLIQFLQ | 155 |
| | . . . . . | |
| 210 | KKAKNLDAITTPDPTTNASLLTKLQAQNQWLQDMTTHLILRSFKEFLQSS | 259 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 156 | KKAKNLDAITTPDPTTNASLLTKLQAQNQWLQDMTTHLILRSFKEFLQSS | 205 |
| 260 | LRALRQM | 266 |
| | \|\|\|\|\|\|\| | |
| 206 | LRALRQM | 212 |

| Sequence name: IL6_HUMAN (SEQ ID NO:193) | | | |
|---|---|---|---|
| Sequence documentation: | | | |
| Alignment of: S56892_PEA_1_P8 (SEQ ID NO:195) × | | | |
| IL6_HUMAN (SEQ ID NO:193) . . | | | |
| Alignment segment 1/1: | | | |
| Quality: | 1526.00 | Escore: | 0 |
| Matching length: | 157 | Total length: | 157 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MNSFSTSAFGPVAFSLGLLLVLPAAFPAPVPPGEDSKDVAAPHRQPLTSS | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MNSFSTSAFGPVAFSLGLLLVLPAAFPAPVPPGEDSKDVAAPHRQPLTSS | 50 |
| | . . . . . | |
| 51 | ERIDKQIRYILDGISALRKETCNKSNMCESSKEALAENNLNLPKMAEKDG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ERIDKQIRYILDGISALRKETCNKSNMCESSKEALAENNLNLPKMAEKDG | 100 |
| | . . . . . | |
| 101 | CFQSGFNEETCLVKIITGLLEFEVYLEYLQNRFESSEEQARAVQMSTKVL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | CFQSGFNEETCLVKIITGLLEFEVYLEYLQNRFESSEEQARAVQMSTKVL | 150 |
| 151 | IQFLQKK | 157 |
| | \|\|\|\|\|\|\| | |
| 151 | IQFLQKK | 157 |

| Sequence name: IL6_HUMAN (SEQ ID NO:193) | | | |
|---|---|---|---|
| Sequence documentation: | | | |
| Alignment of: S56892_PEA_1_P9 (SEQ ID NO:196) × | | | |
| IL6_HUMAN (SEQ ID NO:193) . . | | | |
| Alignment segment 1/1: | | | |
| Quality: | 1490.00 | Escore: | 0 |
| Matching length: | 163 | Total length: | 212 |
| Matching Percent | 100.00 | Matching Percent | 100.00 |
| Similarity: | | Identity: | |
| Total Percent | 76.89 | Total Percent | 76.89 |
| Similarity: | | Identity: | |
| Gaps: | 1 | | |

| Alignment: | | |
|---|---|---|
| | . . . . . | |
| 1 | MNSFSTSAFGPVAFSLGLLLVLPAAFPAPVPPGEDSKDVAAPHRQPLTSS | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MNSFSTSAFGPVAFSLGLLLVLPAAFPAPVPPGEDSKDVAAPHRQPLTSS | 50 |
| | . . . . . | |
| 51 | ERIDKQIRYILDGISALRKETCNKSNMCESSKEALAENNLNLPKMAEKDG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ERIDKQIRYILDGISALRKETCNKSNMCESSKEALAENNLNLPKMAEKDG | 100 |
| | . . . . . | |
| 101 | CFQSGFNE.......................................... | 108 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | CFQSGFNEETCLVKIITGLLEFEVYLEYLQNRFESSEEQARAVQMSTKVL | 150 |
| | . . . . . | |
| 109 | .......AKNLDAITTPDPTTNASLLTKLQAQNQWLQDMTTHLILRSFKE | 151 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | IQFLQKKAKNLDAITTPDPTTNASLLTKLQAQNQWLQDMTTHLILRSFKE | 200 |
| | . | |
| 152 | FLQSSLRALRQM | 163 |
| | \|\|\|\|\|\|\|\|\|\|\|\| | |
| 201 | FLQSSLRALRQM | 212 |

| Sequence name: IL6_HUMAN (SEQ ID NO:193) | | | |
|---|---|---|---|
| Sequence documentation: | | | |
| Alignment of: S56892_PEA_1_P11 (SEQ ID NO:197) × | | | |
| IL6_HUMAN (SEQ ID NO:193) . . | | | |
| Alignment segment 1/1: | | | |
| Quality: | 733.00 | Escore: | 0 |
| Matching length: | 77 | Total length: | 77 |
| Matching Percent | 100.00 | Matching Percent | 98.70 |
| Similarity: | | Identity: | |
| Total Percent | 100.00 | Total Percent | 98.70 |
| Similarity: | | Identity: | |
| Gaps: | 0 | | |

| Alignment: | | |
|---|---|---|
| | . . . . . | |
| 1 | MNSFSTSAFGPVAFSLGLLLVLPAAFPAPVPPGEDSKDVAAPHRQPLTSS | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MNSFSTSAFGPVAFSLGLLLVLPAAFPAPVPPGEDSKDVAAPHRQPLTSS | 50 |
| | . . | |
| 51 | ERIDKQIRYILDGISALRKETCNKSNI | 77 |
| | \|\|\|\|\|\|\|\|\|\|\|\|\|\|\|\|\|\|\|\|\|\|\|\|\|\|\|\| | |
| 51 | ERIDKQIRYILDGISALRKETCNKSNM | 77 |

### Description for Cluster HSIGFACI

Cluster HSIGFACI features 6 transcript(s) and 16 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HSIGFACI_PEA_1_T9 | 198 |
| HSIGFACI_PEA_1_T10 | 199 |
| HSIGFACI_PEA_1_T12 | 200 |
| HSIGFACI_PEA_1_T15 | 201 |
| HSIGFACI_PEA_1_T16 | 202 |
| HSIGFACI_PEA_1_T17 | 203 |

TABLE 2
Segments of interest

| Segment Name | Sequence ID No. |
|---|---|
| HSIGFACI_PEA_1_node_0 | 204 |
| HSIGFACI_PEA_1_node_2 | 205 |
| HSIGFACI_PEA_1_node_6 | 206 |
| HSIGFACI_PEA_1_node_9 | 207 |
| HSIGFACI_PEA_1_node_11 | 208 |
| HSIGFACI_PEA_1_node_14 | 209 |
| HSIGFACI_PEA_1_node_19 | 210 |
| HSIGFACI_PEA_1_node_20 | 211 |
| HSIGFACI_PEA_1_node_21 | 212 |
| HSIGFACI_PEA_1_node_24 | 213 |
| HSIGFACI_PEA_1_node_25 | 214 |
| HSIGFACI_PEA_1_node_26 | 215 |
| HSIGFACI_PEA_1_node_27 | 216 |
| HSIGFACI_PEA_1_node_13 | 217 |
| HSIGFACI_PEA_1_node_22 | 218 |
| HSIGFACI_PEA_1_node_23 | 219 |

TABLE 3
Proteins of interest

| | Sequence | |
|---|---|---|
| Protein Name | ID No. | Corresponding Transcript(s) |
| HSIGFACI_PEA_1_P5 | 225 | HSIGFACI_PEA_1_T9 (SEQ |
| | | ID NO: 198) |
| HSIGFACI_PEA_1_P2 | 226 | HSIGFACI_PEA_1_T12 |
| | | (SEQ ID NO: 200) |
| HSIGFACI_PEA_1_P6 | 227 | HSIGFACI_PEA_1_T15 |
| | | (SEQ ID NO: 201) |
| HSIGFACI_PEA_1_P1 | 228 | HSIGFACI_PEA_1_T16 |
| | | (SEQ ID NO: 202) |
| HSIGFACI_PEA_1_P7 | 229 | HSIGFACI_PEA_1_T10 |
| | | (SEQ ID NO: 199) |
| HSIGFACI_PEA_1_P8 | 230 | HSIGFACI_PEA_1_T17 |
| | | (SEQ ID NO: 203) |

These sequences are variants of the known protein Insulin-like growth factor IB precursor (SEQ ID NO:220) (SwissProt accession identifier IGFB_HUMAN; known also according to the synonyms IGF-IB; Somatomedin C), referred to herein as the previously known protein.

Protein Insulin-like growth factor IB precursor (SEQ ID NO:220) is known or believed to have the following function(s): insulin-like growth factors, isolated from plasma, are structurally and functionally related to insulin but have a much higher growth-promoting activity. The sequence for protein Insulin-like growth factor IB precursor is given at the end of the application, as “Insulin-like growth factor IB precursor amino acid sequence” (SEQ ID NO:220). Known polymorphisms for this sequence are as shown in Table 4.

TABLE 4
Amino acid mutations for Known Protein

| SNP position(s) on | |
|---|---|
| amino acid sequence | Comment |
| 187 | A -> D (in dbSNP: 6213). /FTId = VAR_013945. |

Protein Insulin-like growth factor IB precursor (SEQ ID NO:220) localization is believed to be Secreted.

The mean serum IGF I levels of controls and early-stage endometriosis patients were significantly lower than those in the late stage of endometrosis (Gurgan et al, J Reprod Med. 1999 May; 44(5):450-4). Variants of this cluster are suitable as diagnostic markers for endometriosis.

The previously known protein also has the following indication(s) and/or potential therapeutic use(s): Amyotrophic lateral sclerosis; Neuropathy; Osteoporosis; Wound healing; Cancer; Diabetes; Neuropathy, diabetic. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Insulin like growth factor 1 agonist; Insulin like growth factor 2 agonist; Insulin like growth factor agonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Ophthalmological; Growth hormone; Vulnerary; Osteoporosis treatment; Neuroprotective; Antidiabetic; Nutritional supplement; Antiarthritic; Multiple sclerosis treatment; Neurological; Symptomatic antidiabetic.

The following GO Annotation(s) apply to the previously known protein. The following annotation(s) were found: skeletal development; DNA replication; cell motility; signal transduction; RAS protein signal transduction; muscle development; physiological processes; positive control of cell proliferation; glycolate metabolism, which are annotation(s) related to Biological Process; insulin-like growth factor receptor ligand; hormone; growth factor, which are annotation(s) related to Molecular Function; and extracellular, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster HSIGFACI features 6 transcript(s), which were listed in Table 1 above. These transcript(s) encode for protein(s) which are variant(s) of protein Insulin-like growth factor IB precursor (SEQ ID NO:220). A description of each variant protein according to the present invention is now provided.

Variant protein HSIGFACI_PEA_{—}1_P5 (SEQ ID NO:225) according to the present invention has an amino acid sequence as given at the end of the application; it is encoded by transcript(s) HSIGFACI_PEA_{—}1_T9 (SEQ ID NO:198). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P5 (SEQ ID NO:225) and Q9NP10 (SEQ ID NO:222):

1. An isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPTVK (SEQ ID NO:483) corresponding to amino acids 1-7 of HSIGFACI_PEA_{—}1_P5 (SEQ ID NO:225), a second amino acid sequence being at least 90% homologous to MHTMSSSHLFYLALCLLTFTSSATAGPETLCGAELVDALQFVCGDRGFYFNKPTGYGSS SRRAPQTGIVDECCFRSCDLRRLEMYCAPLKPAKSARSVRAQRHTDMPKTQK corresponding to amino acids 1-111 of Q9NP10 (SEQ ID NO:222), which also corresponds to amino acids 8-118 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence, second amino acid sequence and third amino acid sequence are contiguous and in a sequential order.

2. An isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPTVK (SEQ ID NO:483) of HSIGFACI_PEA_{—}1_P5 (SEQ ID NO:225).

3. An isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

Comparison report between HSIGFACI_PEA_{—}1_P5 (SEQ ID NO:225) and Q13429 (SEQ ID NO:224):

1. An isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), and a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQKYQP PSTNKNTKSQRRKGSTFEERK corresponding to amino acids 3-139 of Q13429 (SEQ ID NO:224), which also corresponds to amino acids 6-142 of HSIGFACI_PEA_{—}1_P5 (SEQ ID NO:225), wherein said first amino acid sequence and second amino acid sequence are contiguous and in a sequential order.

2. An isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

Comparison report between HSIGFACI_PEA_{—}1_P5 (SEQ ID NO:225) and IGFB_HUMAN (SEQ ID NO:220):

1. An isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQKYQP PSTNKNTKSQRRKG corresponding to amino acids 22-151 of IGFB_HUMAN (SEQ ID NO:220), which also corresponds to amino acids 6-135 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence STFEERK corresponding to amino acids 136-142 of HSIGFACI_PEA_{—}1_P5 (SEQ ID NO:225), wherein said first amino acid sequence, second amino acid sequence and third amino acid sequence are contiguous and in a sequential order.

2. An isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

3. An isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence STFEERK in HSIGFACI_PEA_{—}1_P5 (SEQ ID NO:225).

Comparison report between HSIGFACI_PEA_{—}1_P5 (SEQ ID NO:225) and Q14620 (SEQ ID NO:221):

1. An isolated chimeric polypeptide encoding for HSIGFACI_PEA_{—}1_P5 (SEQ ID NO:225), comprising a first amino acid sequence being at least 90% homologous to MITPTVKMHTMSSSHLFYLALCLLTFTSSATAGPETLCGAELVDALQFVCGDRGFYFNK PTGYGSSSRRAPQTGIVDECCFRSCDLRRLEMYCAPLKPAKSARSVRAQRHTDMPKTQ K corresponding to amino acids 1-118 of Q14620 (SEQ ID NO:221), which also corresponds to amino acids 1-118 of HSIGFACI_PEA_{—}1_P5 (SEQ ID NO:225), and a second amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence and second amino acid sequence are contiguous and in a sequential order.

2. An isolated polypeptide encoding for a tail of HSIGFACI_PEA_L P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

Comparison report between HSIGFACI_PEA_{—}1_P5 (SEQ ID NO:225) and IGFA_HUMAN (SEQ ID NO:223):

1. An isolated chimeric polypeptide encoding for HSIGFACI_PEA_{—}1_P5 (SEQ ID NO:225), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P5 (SEQ ID NO:225), a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQK corresponding to amino acids 22-134 of IGFA_HUMAN (SEQ ID NO:223), which also corresponds to amino acids 6-118 of HSIGFACI_PEA_{—}1_P5 (SEQ ID NO:225), and a third amino acid sequence being at least 70%, optionally at least 80%, preferably at least 85%, more preferably at least 90% and most preferably at least 95% homologous to a polypeptide having the sequence YQPPSTNKNTKSQRRKGSTFEERK (SEQ ID NO:484) corresponding to amino acids 119-142 of HSIGFACI_PEA_{—}1_P5 (SEQ ID NO:225), wherein said first amino acid sequence, second amino acid sequence and third amino acid sequence are contiguous and in a sequential order.

2. An isolated polypeptide encoding for a head of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P5 (SEQ ID NO:225).

3. An isolated polypeptide encoding for a tail of HSIGFACI_PEA_{—}1_P5 (SEQ ID NO:225), comprising a polypeptide being at least 70%, optionally at least about 80%, preferably at least about 85%, more preferably at least about 90% and most preferably at least about 95% homologous to the sequence YQPPSTNKNTKSQRRKGSTFEERK (SEQ ID NO:484) in HSIGFACI_PEA_{—}1_P5 (SEQ ID NO:225).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P5 (SEQ ID NO:225) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein HSIGFACI_PEA_{—}1_P5 (SEQ ID NO:225) Sequence provides support for the deduced sequence of this variant protein according to the present invention).

TABLE 5
Amino acid mutations

| SNP position(s) on amino acid | Alternative | |
|---|---|---|
| sequence | amino acid(s) | Previously known SNP? |
| 28 | S -> N | No |

Variant protein HSIGFACI_PEA_{—}1_P5 (SEQ ID NO:225) is encoded by the following transcript(s): HSIGFACI_PEA_{—}1_T9 (SEQ ID NO:198), for which the sequence(s) is/are given at the end of the application. The coding portion of transcript HSIGFACI_PEA_{—}1_T9 (SEQ ID NO:198) is shown in bold; this coding portion starts at position 835 and ends at position 1260. Transcript also has the following SNPs as listed in Table 6 (given according to their position on the nucleotide sequence, with the alternative nucleic acid listed; the last column indicates whether the SNP is known or not; the presence of known SNPs in variant protein HSIGFACI_PEA_{—}1_P5 (SEQ ID NO:225) Sequence provides support for the deduced sequence of this variant protein according to the present invention).

TABLE 6
Nucleic acid SNPs

| SNP position on nucleotide | Alternative | |
|---|---|---|
| sequence | nucleic acid | Previously known SNP? |
| 917 | G -> A | No |
| 942 | G -> A | Yes |
| 1071 | C -> T | Yes |
| 1324 | G -> A | Yes |
| 1403 | A -> G | Yes |
| 1450 | A -> | Yes |
| 1558 | A -> T | Yes |
| 1642 | C -> G | Yes |
| 1905 | T -> A | Yes |
| 2050 | A -> | Yes |
| 2068 | T -> C | Yes |
| 2081 | A -> C | Yes |
| 2139 | A -> T | Yes |
| 2221 | T -> A | Yes |
| 2453 | G -> T | Yes |
| 2500 | T -> A | Yes |
| 2518 | G -> C | Yes |
| 2834 | G -> A | Yes |
| 3015 | T -> G | Yes |
| 3021 | C -> G | Yes |
| 3021 | C -> T | Yes |
| 3050 | C -> A | Yes |
| 3067 | T -> C | Yes |
| 3246 | T -> A | Yes |
| 3563 | T -> G | Yes |
| 3662 | A -> G | Yes |
| 3797 | T -> G | Yes |
| 3950 | T -> C | Yes |
| 4014 | G -> A | Yes |
| 4284 | T -> G | Yes |
| 4421 | A -> G | Yes |
| 4524 | C -> G | No |
| 4547 | T -> G | Yes |
| 4690 | C -> T | Yes |
| 5010 | G -> A | Yes |
| 5018 | G -> A | Yes |
| 5027 | G -> A | Yes |
| 5239 | C -> T | Yes |
| 5267 | T -> G | Yes |
| 5273 | A -> G | Yes |
| 5311 | G -> A | Yes |
| 5713 | T -> G | Yes |
| 5729 | A -> T | Yes |
| 5735 | T -> A | Yes |
| 5839 | T -> C | Yes |
| 5855 | G -> C | Yes |
| 6061 | G -> C | Yes |
| 6505 | T -> G | Yes |
| 6573 | T -> G | Yes |
| 6689 | T -> C | Yes |
| 6764 | G -> A | Yes |
| 6808 | A -> T | Yes |
| 6853 | T -> G | Yes |
| 6912 | G -> A | Yes |
| 6974 | T -> G | Yes |
| 6982 | G -> T | Yes |
| 7205 | T -> G | No |
| 7396 | G -> A | Yes |
| 7475 | C -> T | Yes |
| 7614 | T -> C | Yes |
| 7687 | C -> T | Yes |
| 7736 | G -> C | Yes |
| 7810 | C -> A | Yes |
| 7825 | T -> G | Yes |

Variant protein HSIGFACI_PEA_{—}1_P2 (SEQ ID NO:226) according to the present invention has an amino acid sequence as given at the end of the application; it is encoded by transcript(s) HSIGFACI_PEA_{—}1_T12 (SEQ ID NO:200). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P2 (SEQ ID NO:226) and IGFA_HUMAN (SEQ ID NO:223):

1. An isolated chimeric polypeptide encoding for HSIGFACI_PEA_{—}1_P2 (SEQ ID NO:226), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P2 (SEQ ID NO:226), and a second amino acid sequence being at least 90% homologous to VKMHTMSSSHLFYLALCLLTFTSSATAGPETLCGAELVDALQFVCGDRGFYFNKPTGY GSSSRRAPQTGIVDECCFRSCDLRRLEMYCAPLKPAKSARSVRAQRHTDMPKTQKEVH LKNASRGSAGNKNYRM (SEQ ID NO:487) corresponding to amino acids 22-153 of IGFA_HUMAN (SEQ ID NO:223), which also corresponds to amino acids 6-137 of HSIGFACI_PEA_{—}1_P2 (SEQ ID NO:226), wherein said first amino acid sequence and second amino acid sequence are contiguous and in a sequential order.

2. An isolated polypeptide encoding for a head of HSIGFACI_PEA_{—}1_P2 (SEQ ID NO:226), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P2 (SEQ ID NO:226).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P2 (SEQ ID NO:226) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HSIGFACI_PEA_{—}1_P2 (SEQ ID NO:226) Sequence provides support for the deduced sequence of this variant protein according to the present invention).

TABLE 7
Amino acid mutations

| SNP position(s) on amino acid | Alternative | |
|---|---|---|
| sequence | amino acid(s) | Previously known SNP? |
| 28 | S -> N | No |

Variant protein HSIGFACI_PEA_{—}1_P2 (SEQ ID NO:226) is encoded by the following transcript(s): HSIGFACI_PEA_{—}1_T12 (SEQ ID NO:200), for which the sequence(s) is/are given at the end of the application. The coding portion of transcript HSIGFACI_PEA_{—}1_T12 (SEQ ID NO:200) is shown in bold; this coding portion starts at position 835 and ends at position 1245. The transcript also has the following SNPs as listed in Table 8 (given according to their position on the nucleotide sequence, with the alternative nucleic acid listed; the last column indicates whether the SNP is known or not; the presence of known SNPs in variant protein HSIGFACI_PEA_{—}1_P2 (SEQ ID NO:226) Sequence provides support for the deduced sequence of this variant protein according to the present invention).

TABLE 8
Nucleic acid SNPs

| SNP position on nucleotide | Alternative | |
|---|---|---|
| sequence | nucleic acid | Previously known SNP? |
| 917 | G -> A | No |
| 942 | G -> A | Yes |
| 1071 | C -> T | Yes |
| 1275 | G -> A | Yes |
| 1354 | A -> G | Yes |
| 1401 | A -> | Yes |

Variant protein HSIGFACI_PEA_{—}1_P6 (SEQ ID NO: 227) according to the present invention has an amino acid sequence as given at the end of the application; it is encoded by transcript(s) HSIGFACI_PEA_{—}1_T15 (SEQ ID NO:201). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P6 (SEQ ID NO: 227) and IGFA_HUMAN (SEQ ID NO:223):

1. An isolated chimeric polypeptide encoding for HSIGFACI_PEA_{—}1_P6 (SEQ ID NO: 227), comprising a first amino acid sequence being at least 90% homologous to MGKISSLPTQLFKCCFCDFLKVKMHTMSSSHLFYLALCLLTFTSSATAGPETLCGAELV DALQFVCGDRGFYFNKPTGYGSSSRRAPQTGIVDECCFRSCDLRRLEMYCAPLKPAKS ARSVRAQRHTDMPKTQK corresponding to amino acids 1-134 of IGFA_HUMAN (SEQ ID NO:223), which also corresponds to amino acids 1-134 of HSIGFACI_PEA_{—}1_P6 (SEQ ID NO: 227), and a second amino acid sequence being at least 70%, optionally at least 80%, preferably at least 85%, more preferably at least 90% and most preferably at least 95% homologous to a polypeptide having the sequence YQPPSTNKNTKSQRRKGWPKTHPGGEQKEGTEASLQIRGKKKEQRREIGSRNAECRGK KGK (SEQ ID NO:486) corresponding to amino acids 135-195 of HSIGFACI_PEA_{—}1_P6 (SEQ ID NO: 227), wherein said first amino acid sequence and second amino acid sequence are contiguous and in a sequential order.

2. An isolated polypeptide encoding for a tail of HSIGFACI_PEA_{—}1_P6 (SEQ ID NO: 227), comprising a polypeptide being at least 70%, optionally at least about 80%, preferably at least about 85%, more preferably at least about 90% and most preferably at least about 95% homologous to the sequence YQPPSTNKNTKSQRRKGWPKTHPGGEQKEGTEASLQIRGKKKEQRREIGSRNAECRGK KGK (SEQ ID NO:486) in HSIGFACI_PEA_{—}1_P6 (SEQ ID NO: 227).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P6 (SEQ ID NO: 227) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HSIGFACI_PEA_{—}1_P6 (SEQ ID NO: 227) Sequence provides support for the deduced sequence of this variant protein according to the present invention).

TABLE 9
Amino acid mutations

| SNP position(s) on amino acid | Alternative | |
|---|---|---|
| sequence | amino acid(s) | Previously known SNP? |
| 2 | G -> E | Yes |
| 44 | S -> N | No |
| 187 | A -> D | Yes |

Variant protein HSIGFACI_PEA_{—}1_P6 (SEQ ID NO: 227) is encoded by the following transcript(s): HSIGFACI_PEA_{—}1_T15 (SEQ ID NO:201), for which the sequence(s) is/are given at the end of the application. The coding portion of transcript HSIGFACI_PEA_{—}1_T15 (SEQ ID NO:201) is shown in bold; this coding portion starts at position 266 and ends at position 850. The transcript also has the following SNPs as listed in Table 10 (given according to their position on the nucleotide sequence, with the alternative nucleic acid listed; the last column indicates whether the SNP is known or not; the presence of known SNPs in variant protein HSIGFACI_PEA_{—}1_P6 (SEQ ID NO: 227) Sequence provides support for the deduced sequence of this variant protein according to the present invention).

TABLE 10
Nucleic acid SNPs

| SNP position on nucleotide | Alternative | |
|---|---|---|
| sequence | nucleic acid | Previously known SNP? |
| 254 | A -> T | Yes |
| 270 | G -> A | Yes |
| 396 | G -> A | No |
| 421 | G -> A | Yes |
| 550 | C -> T | Yes |
| 825 | C -> A | Yes |
| 1210 | T -> C | Yes |
| 1351 | C -> T | No |

Variant protein HSIGFACI_PEA_{—}1_P1 (SEQ ID NO:228) according to the present invention has an amino acid sequence as given at the end of the application; it is encoded by transcript(s) HSIGFACI_PEA_{—}1_T16 (SEQ ID NO:202). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P1 (SEQ ID NO:228) and IGFB_HUMAN (SEQ ID NO:220):

1. An isolated chimeric polypeptide encoding for HSIGFACI_PEA_{—}1_P1 (SEQ ID NO:228), comprising a first amino acid sequence being at least 90% homologous to MGKISSLPTQLFKCCFCDFLKVKMHTMSSSHLFYLALCLLTFTSSATAGPETLCGAELV DALQFVCGDRGFYFNKPTGYGSSSRRAPQTGIVDECCFRSCDLRRLEMYCAPLKPAKS ARSVRAQRHTDMPKTQK corresponding to amino acids 1-134 of IGFB_HUMAN (SEQ ID NO:220), which also corresponds to amino acids 1-134 of HSIGFACI_PEA_{—}1_P1 (SEQ ID NO:228), and a second amino acid sequence being at least 70%, optionally at least 80%, preferably at least 85%, more preferably at least 90% and most preferably at least 95% homologous to a polypeptide having the sequence EVHLKNASRGSAGNKNYRM (SEQ ID NO:487) corresponding to amino acids 135-153 of HSIGFACI_PEA_{—}1_P1 (SEQ ID NO:228), wherein said first amino acid sequence and second amino acid sequence are contiguous and in a sequential order.

2. An isolated polypeptide encoding for a tail of HSIGFACI_PEA_{—}1_P1 (SEQ ID NO:228), comprising a polypeptide being at least 70%, optionally at least about 80%, preferably at least about 85%, more preferably at least about 90% and most preferably at least about 95% homologous to the sequence EVHLKNASRGSAGNKNYRM (SEQ ID NO:487) in HSIGFACI_PEA_{—}1_P1 (SEQ ID NO:228).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P1 (SEQ ID NO:228) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HSIGFACI_PEA_{—}1_P1 (SEQ ID NO:228) Sequence provides support for the deduced sequence of this variant protein according to the present invention).

TABLE 11
Amino acid mutations

| SNP position(s) on amino acid | | Previously |
|---|---|---|
| sequence | Alternative amino acid(s) | known SNP? |
| 2 | G -> E | Yes |
| 44 | S -> N | No |

Variant protein HSIGFACI_PEA_{—}1_P1 (SEQ ID NO:228) is encoded by the following transcript(s): HSIGFACI_PEA_{—}1_T16 (SEQ ID NO:202), for which the sequence(s) is/are given at the end of the application. The coding portion of transcript HSIGFACI_PEA_{—}1_T16 (SEQ ID NO:202) is shown in bold; this coding portion starts at position 266 and ends at position 724. The transcript also has the following SNPs as listed in Table 12 (given according to their position on the nucleotide sequence, with the alternative nucleic acid listed; the last column indicates whether the SNP is known or not; the presence of known SNPs in variant protein HSIGFACI_PEA_{—}1_P1 (SEQ ID NO:228) Sequence provides support for the deduced sequence of this variant protein according to the present invention).

TABLE 12
Nucleic acid SNPs

| SNP position on nucleotide | | Previously |
|---|---|---|
| sequence | Alternative nucleic acid | known SNP? |
| 254 | A -> T | Yes |
| 270 | G -> A | Yes |
| 396 | G -> A | No |
| 421 | G -> A | Yes |
| 550 | C -> T | Yes |
| 754 | G -> A | Yes |
| 833 | A -> G | Yes |
| 880 | A -> | Yes |

Variant protein HSIGFACI_PEA_{—}1_P7 (SEQ ID NO:229) according to the present invention has an amino acid sequence as given at the end of the application; it is encoded by transcript(s) HSIGFACI_PEA_{—}1_T10 (SEQ ID NO:199). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P7 (SEQ ID NO:229) and IGFB_HUMAN (SEQ ID NO:220):

1. An isolated chimeric polypeptide encoding for HSIGFACI_PEA_{—}1_P7 (SEQ ID NO:229), comprising a first amino acid sequence being at least 90% homologous to MGKISSLPTQLFKCCFCDFLKVKMHTMSSSHLFYLALCLLTFTSSATAGPETLCGAELV DALQFVCGDRGFYF corresponding to amino acids 1-73 of IGFB_HUMAN (SEQ ID NO:220), which also corresponds to amino acids 1-73 of HSIGFACI_PEA_{—}1_P7 (SEQ ID NO:229), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 74-108 of HSIGFACI_PEA_{—}1_P7 (SEQ ID NO:229), wherein said first amino acid sequence and second amino acid sequence are contiguous and in a sequential order.

2. An isolated polypeptide encoding for a tail of HSIGFACI_PEA_{—}1_P7 (SEQ ID NO:229), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P7 (SEQ ID NO:229).

Comparison report between HSIGFACI_PEA_{—}1_P7 (SEQ ID NO:229) and IGFA_HUMAN (SEQ ID NO:223):

1. An isolated chimeric polypeptide encoding for HSIGFACI_PEA_{—}1_P7 (SEQ ID NO:229), comprising a first amino acid sequence being at least 90% homologous to MGKISSLPTQLFKCCFCDFLKVKMHTMSSSHLFYLALCLLTFTSSATAGPETLCGAELV DALQFVCGDRGFYF corresponding to amino acids 1-73 of IGFA_HUMAN (SEQ ID NO:223), which also corresponds to amino acids 1-73 of HSIGFACI_PEA_{—}1_P7 (SEQ ID NO:229), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 74-108 of HSIGFACI_PEA_{—}1_P7 (SEQ ID NO:229), wherein said first amino acid sequence and second amino acid sequence are contiguous and in a sequential order.

2. An isolated polypeptide encoding for a tail of HSIGFACI_PEA_{—}1_P7 (SEQ ID NO:229), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P7 (SEQ ID NO:229).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P7 (SEQ ID NO:229) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HSIGFACI_PEA_{—}1_P7 (SEQ ID NO:229) Sequence provides support for the deduced sequence of this variant protein according to the present invention).

TABLE 13
Amino acid mutations

| SNP position(s) on amino acid | | Previously |
|---|---|---|
| sequence | Alternative amino acid(s) | known SNP? |
| 2 | G -> E | Yes |
| 44 | S -> N | No |

Variant protein HSIGFACI_PEA_{—}1_P7 (SEQ ID NO:229) is encoded by the following transcript(s): HSIGFACI_PEA_{—}1_T10 (SEQ ID NO:199), for which the sequence(s) is/are given at the end of the application. The coding portion of transcript HSIGFACI_PEA_{—}1_T10 (SEQ ID NO:199) is shown in bold; this coding portion starts at position 266 and ends at position 589. The transcript also has the following SNPs as listed in Table 14 (given according to their position on the nucleotide sequence, with the alternative nucleic acid listed; the last column indicates whether the SNP is known or not; the presence of known SNPs in variant protein HSIGFACI_PEA_{—}1_P7 (SEQ ID NO:229) Sequence provides support for the deduced sequence of this variant protein according to the present invention).

TABLE 14
Nucleic acid SNPs

| SNP position on nucleotide | | Previously |
|---|---|---|
| sequence | Alternative nucleic acid | known SNP? |
| 254 | A -> T | Yes |
| 270 | G -> A | Yes |
| 396 | G -> A | No |
| 421 | G -> A | Yes |
| 687 | G -> A | Yes |
| 810 | A -> G | Yes |
| 850 | A -> C | Yes |
| 853 | G -> A | Yes |
| 859 | A -> C | Yes |
| 909 | C -> T | Yes |
| 1096 | C -> T | Yes |
| 1300 | G -> A | Yes |
| 1379 | A -> G | Yes |
| 1426 | A -> | Yes |
| 1534 | A -> T | Yes |
| 1618 | C -> G | Yes |
| 1881 | T -> A | Yes |
| 2026 | A -> | Yes |
| 2044 | T -> C | Yes |
| 2057 | A -> C | Yes |
| 2115 | A -> T | Yes |
| 2197 | T -> A | Yes |
| 2429 | G -> T | Yes |
| 2476 | T -> A | Yes |
| 2494 | G -> C | Yes |
| 2810 | G -> A | Yes |
| 2991 | T -> G | Yes |
| 2997 | C -> G | Yes |
| 2997 | C -> T | Yes |
| 3026 | C -> A | Yes |
| 3043 | T -> C | Yes |
| 3222 | T -> A | Yes |
| 3539 | T -> G | Yes |
| 3638 | A -> G | Yes |
| 3773 | T -> G | Yes |
| 3926 | T -> C | Yes |
| 3990 | G -> A | Yes |
| 4260 | T -> G | Yes |
| 4397 | A -> G | Yes |
| 4500 | C -> G | No |
| 4523 | T -> G | Yes |
| 4666 | C -> T | Yes |
| 4986 | G -> A | Yes |
| 4994 | G -> A | Yes |
| 5003 | G -> A | Yes |
| 5215 | C -> T | Yes |
| 5243 | T -> G | Yes |
| 5249 | A -> G | Yes |
| 5287 | G -> A | Yes |
| 5689 | T -> G | Yes |
| 5705 | A -> T | Yes |
| 5711 | T -> A | Yes |
| 5815 | T -> C | Yes |
| 5831 | G -> C | Yes |
| 6037 | G -> C | Yes |
| 6481 | T -> G | Yes |
| 6549 | T -> G | Yes |
| 6665 | T -> C | Yes |
| 6740 | G -> A | Yes |
| 6784 | A -> T | Yes |
| 6829 | T -> G | Yes |
| 6888 | G -> A | Yes |
| 6950 | T -> G | Yes |
| 6958 | G -> T | Yes |
| 7181 | T -> G | No |
| 7372 | G -> A | Yes |
| 7451 | C -> T | Yes |
| 7590 | T -> C | Yes |
| 7663 | C -> T | Yes |
| 7712 | G -> C | Yes |
| 7786 | C -> A | Yes |
| 7801 | T -> G | Yes |

Variant protein HSIGFACI_PEA_{—}1_P8 (SEQ ID NO:230) according to the present invention has an amino acid sequence as given at the end of the application; it is encoded by transcript(s) HSIGFACI_PEA_{—}1_T17 (SEQ ID NO:203). An alignment is given to the known protein (Insulin-like growth factor IB precursor (SEQ ID NO:220)) at the end of the application. One or more alignments to one or more previously published protein sequences are given at the end of the application. A brief description of the relationship of the variant protein according to the present invention to each such aligned protein is as follows:

Comparison report between HSIGFACI_PEA_{—}1_P8 (SEQ ID NO:230) and Q9NP10 (SEQ ID NO:222):

1. An isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPTVK (SEQ ID NO:483) corresponding to amino acids 1-7 of HSIGFACI_PEA_{—}1_P8 (SEQ ID NO:230), a second amino acid sequence being at least 90% homologous to MHTMSSSHLFYLALCLLTFTSSATAGPETLCGAELVDALQFVCGDRGFYF corresponding to amino acids 1-50 of Q9NP10 (SEQ ID NO:222), which also corresponds to amino acids 8-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

2. An isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPTVK (SEQ ID NO:483) of HSIGFACI_PEA_{—}1_P8 (SEQ ID NO:230).

3. An isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

Comparison report between HSIGFACI_PEA_{—}1_P8 (SEQ ID NO:230) and Q13429 (SEQ ID NO:224):

1. An isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 3-54 of Q13429 (SEQ ID NO:224), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

2. An isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

3. An isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

Comparison report between HSIGFACI_PEA_{—}1_P8 (SEQ ID NO:230) and Q14620 (SEQ ID NO:221):

1. An isolated chimeric polypeptide encoding for HSIGFACI_PEA_{—}1_P8 (SEQ ID NO:230), comprising a first amino acid sequence being at least 90% homologous to MITPTVKMHTMSSSSHLFYLALCLLTFTSSATAGPETLCGAELVDALQFVCGDRGFYF corresponding to amino acids 1-57 of Q14620 (SEQ ID NO:221), which also corresponds to amino acids 1-57 of HSIGFACI_PEA_{—}1_P8 (SEQ ID NO:230), and a second amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence and second amino acid sequence are contiguous and in a sequential order.

2. An isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

Comparison report between HSIGFACI_PEA_{—}1_P8 (SEQ ID NO:230) and IGFB_HUMAN (SEQ ID NO:220):

1. An isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFB_HUMAN (SEQ ID NO:220), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

2. An isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

3. An isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

Comparison report between HSIGFACI_PEA_{—}1_P8 (SEQ ID NO:230) and IGFA_HUMAN (SEQ ID NO:223):

1. An isolated chimeric polypeptide encoding for HSIGFACI_PEA_{—}1_P8 (SEQ ID NO:230), comprising a first amino acid sequence being at least 70%, optionally at least 80%, preferably at least 85%, more preferably at least 90% and most preferably at least 95% homologous to a polypeptide having the sequence MITPT (SEQ ID NO:485) corresponding to amino acids 1-5 of HSIGFACI_PEA_{—}1_P8 (SEQ ID NO:230), a second amino acid sequence being at least 90% homologous to VKMHTMSSSHLFYLALCLLTFTSSATAGPETLCGAELVDALQFVCGDRGFYF corresponding to amino acids 22-73 of IGFA_HUMAN (SEQ ID NO:223), which also corresponds to amino acids 6-57 of HSIGFACI_PEA_{—}1_P8 (SEQ ID NO:230), and a third amino acid sequence being at least 70%, optionally at least 80%, preferably at least 85%, more preferably at least 90% and most preferably at least 95% homologous to a polypeptide having the sequence SRKILLKLRSSVARCSGSLLKFQQFERPRQENCLS (SEQ ID NO:488) corresponding to amino acids 58-92 of HSIGFACI_PEA_{—}1_P8 (SEQ ID NO:230), wherein said first amino acid sequence, second amino acid sequence and third amino acid sequence are contiguous and in a sequential order.

2. An isolated polypeptide encoding for a head of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence MITPT (SEQ ID NO:485) of HSIGFACI_PEA_{—}1_P8 (SEQ ID NO:230).

3. An isolated polypeptide encoding for a tail of HSIGFACI_PEA_{—}1_P8 (SEQ ID NO:230), comprising a polypeptide being at least 70%, optionally at least about 80%, preferably at least about 85%, more preferably at least about 90% and most preferably at least about 95% homologous to the sequence SRKILLKLRSSVARCSGSLLKFQQFERPRQENCLS (SEQ ID NO:488) in HSIGFACI_PEA_{—}1_P8 (SEQ ID NO:230).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSIGFACI_PEA_{—}1_P8 (SEQ ID NO:230) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein HSIGFACI_PEA_{—}1_P8 (SEQ ID NO:230) Sequence provides support for the deduced sequence of this variant protein according to the present invention).

TABLE 15
Amino acid mutations

| SNP position(s) on amino acid | | Previously |
|---|---|---|
| sequence | Alternative amino acid(s) | known SNP? |
| 28 | S -> N | No |

Variant protein HSIGFACI_PEA_{—}1_P8 (SEQ ID NO:230) is encoded by the following transcript(s): HSIGFACI_PEA_{—}1_T17 (SEQ ID NO:203), for which the sequence(s) is/are given at the end of the application. The coding portion of transcript HSIGFACI_PEA_{—}1_T17 (SEQ ID NO:203) is shown in bold; this coding portion starts at position 835 and ends at position 1110. The transcript also has the following SNPs as listed in Table 16 (given according to their position on the nucleotide sequence, with the alternative nucleic acid listed; the last column indicates whether the SNP is known or not; the presence of known SNPs in variant protein HSIGFACI_PEA_{—}1_P8 (SEQ ID NO:230) Sequence provides support for the deduced sequence of this variant protein according to the present invention).

TABLE 16
Nucleic acid SNPs

| SNP position on nucleotide | | Previously |
|---|---|---|
| sequence | Alternative nucleic acid | known SNP? |
| 917 | G -> A | No |
| 942 | G -> A | Yes |
| 1208 | G -> A | Yes |
| 1331 | A -> G | Yes |
| 1371 | A -> C | Yes |
| 1374 | G -> A | Yes |
| 1380 | A -> C | Yes |
| 1430 | C -> T | Yes |
| 1617 | C -> T | Yes |
| 1892 | C -> A | Yes |
| 2277 | T -> C | Yes |
| 2418 | C -> T | No |

As noted above, cluster HSIGFACI features 16 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSIGFACI_PEA_{—}1_node_{—}0 (SEQ ID NO:204) according to the present invention is supported by 53 libraries. The number of libraries was determined as previously described. This segment can be found in the following transcript(s): HSIGFACI_PEA_{—}1_T10 (SEQ ID NO:199), HSIGFACI_PEA_{—}1_T15 (SEQ ID NO:201) and HSIGFACI_PEA_{—}1_T16 (SEQ ID NO:202). Table 17 below describes the starting and ending position of this segment on each transcript.

TABLE 17
Segment location on transcripts

| | Segment | |
|---|---|---|
| | starting | Segment |
| Transcript name | position | ending position |
| HSIGFACI_PEA_1_T10 (SEQ ID | 1 | 328 |
| NO: 199) | | |
| HSIGFACI_PEA_1_T15 (SEQ ID | 1 | 328 |
| NO: 201) | | |
| HSIGFACI_PEA_1_T16 (SEQ ID | 1 | 328 |
| NO: 202) | | |

Segment cluster HSIGFACI_PEA_{—}1_node_{—}2 (SEQ ID NO:205) according to the present invention is supported by 14 libraries. The number of libraries was determined as previously described. This segment can be found in the following transcript(s): HSIGFACI_PEA_{—}1_T9 (SEQ ID NO:198), HSIGFACI_PEA_{—}1_T12 (SEQ ID NO:200) and HSIGFACI_PEA_{—}1_T17 (SEQ ID NO:203). Table 18 below describes the starting and ending position of this segment on each transcript.

TABLE 18
Segment location on transcripts

| | Segment | |
|---|---|---|
| | starting | Segment |
| Transcript name | position | ending position |
| HSIGFACI_PEA_1_T9 (SEQ ID | 1 | 849 |
| NO: 198) | | |
| HSIGFACI_PEA_1_T12 (SEQ ID | 1 | 849 |
| NO: 200) | | |
| HSIGFACI_PEA_1_T17 (SEQ ID | 1 | 849 |
| NO: 203) | | |

Segment cluster HSIGFACI_PEA_{—}1_node_{—}6 (SEQ ID NO:206) according to the present invention is supported by 62 libraries. The number of libraries was determined as previously described. This segment can be found in the following transcript(s): HSIGFACI_PEA_{—}1_T9 (SEQ ID NO:198), HSIGFACI_PEA_{—}1_T10 (SEQ ID NO:199), HSIGFACI_PEA_{—}1_T12 (SEQ ID NO:200), HSIGFACI_PEA_{—}1_T15 (SEQ ID NO:201), HSIGFACI_PEA_{—}1_T16 (SEQ ID NO:202) and HSIGFACI_PEA_{—}1_T17 (SEQ ID NO:203). Table 19 below describes the starting and ending position of this segment on each transcript.

TABLE 19
Segment location on transcripts

| | Segment | |
|---|---|---|
| | starting | Segment |
| Transcript name | position | ending position |
| HSIGFACI_PEA_1_T9 (SEQ ID | 850 | 1006 |
| NO: 198) | | |
| HSIGFACI_PEA_1_T10 (SEQ ID | 329 | 485 |
| NO: 199) | | |
| HSIGFACI_PEA_1_T12 (SEQ ID | 850 | 1006 |
| NO: 200) | | |
| HSIGFACI_PEA_1_T15 (SEQ ID | 329 | 485 |
| NO: 201) | | |
| HSIGFACI_PEA_1_T16 (SEQ ID | 329 | 485 |
| NO: 202) | | |
| HSIGFACI_PEA_1_T17 (SEQ ID | 850 | 1006 |
| NO: 203) | | |

Segment cluster HSIGFACI_PEA_{—}1_node_{—}9 (SEQ ID NO:207) according to the present invention is supported by 4 libraries. The number of libraries was determined as previously described. This segment can be found in the following transcript(s): HSIGFACI_PEA_{—}1_T10 (SEQ ID NO:199) and HSIGFACI_PEA_{—}1_T17 (SEQ ID NO:203). Table 20 below describes the starting and ending position of this segment on each transcript.

TABLE 20
Segment location on transcripts

| | Segment | |
|---|---|---|
| | starting | Segment |
| Transcript name | position | ending position |
| HSIGFACI_PEA_1_T10 (SEQ ID | 486 | 1031 |
| NO: 199) | | |
| HSIGFACI_PEA_1_T17 (SEQ ID | 1007 | 1552 |
| NO: 203) | | |

Segment cluster HSIGFACI_PEA_{—}1_node_{—}11 (SEQ ID NO:208) according to the present invention is supported by 53 libraries. The number of libraries was determined as previously described. This segment can be found in the following transcript(s): HSIGFACI_PEA_{—}1_T9 (SEQ ID NO:198), HSIGFACI_PEA_{—}1_T10 (SEQ ID NO:199), HSIGFACI_PEA_{—}1_T12 (SEQ ID NO:200), HSIGFACI_PEA_{—}1_T15 (SEQ ID NO:201), HSIGFACI_PEA_{—}1_T16 (SEQ ID NO:202) and HSIGFACI_PEA_{—}1_T17 (SEQ ID NO:203). Table 21 below describes the starting and ending position of this segment on each transcript.

TABLE 21
Segment location on transcripts

| | Segment | Segment |
|---|---|---|
| | starting | ending |
| Transcript name | position | position |
| HSIGFACI_PEA_1_T9 (SEQ ID | 1007 | 1188 |
| NO: 198) | | |
| HSIGFACI_PEA_1_T10 (SEQ ID | 1032 | 1213 |
| NO: 199) | | |
| HSIGFACI_PEA_1_T12 (SEQ ID | 1007 | 1188 |
| NO: 200) | | |
| HSIGFACI_PEA_1_T15 (SEQ ID | 486 | 667 |
| NO: 201) | | |
| HSIGFACI_PEA_1_T16 (SEQ ID | 486 | 667 |
| NO: 202) | | |
| HSIGFACI_PEA_1_T17 (SEQ ID | 1553 | 1734 |
| NO: 203) | | |

Segment cluster HSIGFACI_PEA_{—}1_node_{—}14 (SEQ ID NO:209) according to the present invention is supported by 22 libraries. The number of libraries was determined as previously described. This segment can be found in the following transcript(s): HSIGFACI_PEA_{—}1_T15 (SEQ ID NO:201) and HSIGFACI_PEA_{—}1_T17 (SEQ ID NO:203). Table 22 below describes the starting and ending position of this segment on each transcript.

TABLE 22
Segment location on transcripts

| | Segment | Segment |
|---|---|---|
| | starting | ending |
| Transcript name | position | position |
| HSIGFACI_PEA_1_T15 (SEQ ID | 717 | 1681 |
| NO: 201) | | |
| HSIGFACI_PEA_1_T17 (SEQ ID | 1784 | 2748 |
| NO: 203) | | |

Segment cluster HSIGFACI_PEA_{—}1_node_{—}19 (SEQ ID NO:210) according to the present invention is supported by 99 libraries. The number of libraries was determined as previously described. This segment can be found in the following transcript(s): HSIGFACI_PEA_{—}1_T9 (SEQ ID NO:198), HSIGFACI_PEA_{—}1_TIO (SEQ ID NO:199), HSIGFACI_PEA_{—}1_T12 (SEQ ID NO:200) and HSIGFACI_PEA_{—}1_T16 (SEQ ID NO:202). Table 23 below describes the starting and ending position of this segment on each transcript.

TABLE 23
Segment location on transcripts

| | Segment | Segment |
|---|---|---|
| | starting | ending |
| Transcript name | position | position |
| HSIGFACI_PEA_1_T9 (SEQ ID | 1238 | 5030 |
| NO: 198) | | |
| HSIGFACI_PEA_1_T10 (SEQ ID | 1214 | 5006 |
| NO: 199) | | |
| HSIGFACI_PEA_1_T12 (SEQ ID | 1189 | 1406 |
| NO: 200) | | |
| HSIGFACI_PEA_1_T16 (SEQ ID | 668 | 885 |
| NO: 202) | | |

Segment cluster HSIGFACI_PEA_{—}1_node_{—}20 (SEQ ID NO:211) according to the present invention is supported by 10 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 24 below describes the starting and ending position of this segment on each transcript.

TABLE 24
Segment location on transcripts

| | Segment | Segment |
|---|---|---|
| | starting | ending |
| Transcript name | position | position |
| HSIGFACI_PEA_1_T9 (SEQ ID | 5031 | 5198 |
| NO: 198) | | |
| HSIGFACI_PEA_1_T10 (SEQ ID | 5007 | 5174 |
| NO: 199) | | |

Segment cluster HSIGFACI_PEA_{—}1_node_{—}21 (SEQ ID NO:212) according to the present invention is supported by 57 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 25 below describes the starting and ending position of this segment on each transcript.

TABLE 25
Segment location on transcripts

| | Segment | Segment |
|---|---|---|
| | starting | ending |
| Transcript name | position | position |
| HSIGFACI_PEA_1_T9 (SEQ ID | 5199 | 7012 |
| NO: 198) | | |
| HSIGFACI_PEA_1_T10 (SEQ ID | 5175 | 6988 |
| NO: 199) | | |

Segment cluster HSIGFACI_PEA_{—}1_node_{—}24 (SEQ ID NO:213) according to the present invention is supported by 57 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 26 below describes the starting and ending position of this segment on each transcript.

TABLE 26
Segment location on transcripts

| | Segment | Segment |
|---|---|---|
| | starting | ending |
| Transcript name | position | position |
| HSIGFACI_PEA_1_T9 (SEQ ID | 7071 | 7396 |
| NO: 198) | | |
| HSIGFACI_PEA_1_T10 (SEQ ID | 7047 | 7372 |
| NO: 199) | | |

Segment cluster HSIGFACI_PEA_{—}1_node_{—}25 (SEQ ID NO:214) according to the present invention is supported by 54 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 27 below describes the starting and ending position of this segment on each transcript.

TABLE 27
Segment location on transcripts

| | Segment | Segment |
|---|---|---|
| | starting | ending |
| Transcript name | position | position |
| HSIGFACI_PEA_1_T9 (SEQ ID | 7397 | 7557 |
| NO: 198) | | |
| HSIGFACI_PEA_1_T10 (SEQ ID | 7373 | 7533 |
| NO: 199) | | |

Segment cluster HSIGFACI_PEA_{—}1_node_{—}26 (SEQ ID NO:215) according to the present invention is supported by 51 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 28 below describes the starting and ending position of this segment on each transcript.

TABLE 28
Segment location on transcripts

| | Segment | Segment |
|---|---|---|
| | starting | ending |
| Transcript name | position | position |
| HSIGFACI_PEA_1_T9 (SEQ ID | 7558 | 7783 |
| NO: 198) | | |
| HSIGFACI_PEA_1_T10 (SEQ ID | 7534 | 7759 |
| NO: 199) | | |

Segment cluster HSIGFACI_PEA_{—}1_node_{—}27 (SEQ ID NO:216) according to the present invention is supported by 37 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 29 below describes the starting and ending position of this segment on each transcript.

TABLE 29
Segment location on transcripts

| | Segment | Segment |
|---|---|---|
| | starting | ending |
| Transcript name | position | position |
| HSIGFACI_PEA_1_T9 (SEQ ID | 7784 | 7935 |
| NO: 198) | | |
| HSIGFACI_PEA_1_T10 (SEQ ID | 7760 | 7911 |
| NO: 199) | | |

According to an optional embodiment of the present invention, short segments related to the above cluster are also provided. These segments are up to about 120 bp in length, and so are included in a separate description.

Segment cluster HSIGFACI_PEA_{—}1_node_{—}13 (SEQ ID NO:217) according to the present invention is supported by 17 libraries. The number of libraries was determined as previously described. This segment can be found in the following transcript(s): HSIGFACI_PEA_{—}1_T9 (SEQ ID NO:198), HSIGFACI_PEA_{—}1_T15 (SEQ ID NO:201) and HSIGFACI_PEA_{—}1_T17 (SEQ ID NO:203). Table 30 below describes the starting and ending position of this segment on each transcript.

TABLE 30
Segment location on transcripts

| | Segment | Segment |
|---|---|---|
| | starting | ending |
| Transcript name | position | position |
| HSIGFACI_PEA_1_T9 (SEQ ID | 1189 | 1237 |
| NO: 198) | | |
| HSIGFACI_PEA_1_T15 (SEQ ID | 668 | 716 |
| NO: 201) | | |
| HSIGFACI_PEA_1_T17 (SEQ ID | 1735 | 1783 |
| NO: 203) | | |

Segment cluster HSIGFACI_PEA_{—}1_node_{—}22 (SEQ ID NO:218) according to the present invention is supported by 23 libraries. The number of libraries was determined as previously described. This segment can be found in the following transcript(s): HSIGFACI_PEA_{—}1_T9 (SEQ ID NO:198) and HSIGFACI_PEA_{—}1_T10 (SEQ ID NO:199). Table 31 below describes the starting and ending position of this segment on each transcript.

TABLE 31
Segment location on transcripts

| | Segment | Segment |
|---|---|---|
| Transcript name | starting position | ending position |
| HSIGFACI_PEA_1_T9 (SEQ ID | 7013 | 7045 |
| NO: 198) | | |
| HSIGFACI_PEA_1_T10 (SEQ ID | 6989 | 7021 |
| NO: 199) | | |

Segment cluster HSIGFACI_PEA_{—}1_node_{—}23 (SEQ ID NO:219) according to the present invention can be found in the following transcript(s): HSIGFACI_PEA_{—}1_T9 (SEQ ID NO:198) and HSIGFACI_PEA_{—}1_T10 (SEQ ID NO:199). Table 32 below describes the starting and ending position of this segment on each transcript.

TABLE 32
Segment location on transcripts

| | Segment | Segment |
|---|---|---|
| Transcript name | starting position | ending position |
| HSIGFACI_PEA_1_T9 (SEQ ID | 7046 | 7070 |
| NO: 198) | | |
| HSIGFACI_PEA_1_T10 (SEQ ID | 7022 | 7046 |
| NO: 199) | | |

Variant protein alignment to the previously known protein:

| Sequence name: Q9NP10 (SEQ ID NO:222) | | | |
|---|---|---|---|
| Sequence documentation: | | | |
| Alignment of: HSIGFACI_PEA_1_P5 (SEQ ID NO:225) × | | | |
| Q9NP10 (SEQ ID NO:222) . . | | | |
| Alignment segment 1/1: | | | |
| Quality: | 1107.00 | Escore: | 0 |
| Matching length: | 111 | Total length: | 111 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 8 | MHTMSSSHLFYLALCLLTFTSSATAGPETLCGAELVDALQFVCGDRGFYF | 57 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MHTMSSSHLFYLALCLLTFTSSATAGPETLCGAELVDALQFVCGDRGFYF | 50 |
| | . . . . . | |
| 58 | NKPTGYGSSSRRAPQTGIVDECCFRSCDLRRLEMYCAPLKPAKSARSVRA | 107 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | NKPTGYGSSSRRAPQTGIVDECCFRSCDLRRLEMYCAPLKPAKSARSVRA | 100 |
| | . | |
| 108 | QRHTDMPKTQK | 118 |
| | \|\|\|\|\|\|\|\|\|\|\| | |
| 101 | QRHTDMPKTQK | 111 |

| Sequence name: Q13429 (SEQ ID NO:224) | | | |
|---|---|---|---|
| Sequence documentation: | | | |
| Alignment of: HSIGFACI_PEA_1_P5 (SEQ ID NO:225) × | | | |
| Q13429 (SEQ ID NO:224) . . | | | |
| Alignment segment 1/1: | | | |
| Quality: | 1369.00 | Escore: | 0 |
| Matching length: | 137 | Total length: | 137 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 3 | VKMHTMSSSHLFYLALCLLTFTSSATAGPETLCGAELVDALQFVCGDRGF | 52 |
| | . . . . . | |
| 56 | YFNKPTGYGSSSRRAPQTGIVDECCFRSCDLRRLEMYCAPLKPAKSARSV | 105 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 53 | YFNKPTGYGSSSRRAPQTGIVDECCFRSCDLRRLEMYCAPLKPAKSARSV | 102 |
| | . . . | |
| 106 | RAQRHTDMPKTQKYQPPSTNKNTKSQRRKGSTFEERK | 142 |
| | \|\|\|\|\|\|\|\|\|\|\|\|\|\|\|\|\|\|\|\|\|\|\|\|\|\|\|\|\|\|\|\|\|\|\|\|\| | |
| 103 | RAQRHTDMPKTQKYQPPSTNKNTKSQRRKGSTFEERK | 139 |

| Sequence name: IGFB_HUMAN (SEQ ID NO:220) | | | |
|---|---|---|---|
| Sequence documentation: | | | |
| Alignment of: HSIGFACI_PEA_1_P5 (SEQ ID NO:225) × | | | |
| IGFB_HUMAN (SEQ ID NO:220) . . | | | |
| Alignment segment 1/1: | | | |
| Quality: | 1300.00 | Escore: | 0 |
| Matching length: | 130 | Total length: | 130 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 22 | VKMHTMSSSHLFYLALCLLTFTSSATAGPETLCGAELVDALQFVCGDRGF | 71 |
| | . . . . . | |
| 56 | YFNKPTGYGSSSRRAPQTGIVDECCFRSCDLRRLEMYCAPLKPAKSARSV | 105 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 72 | YFNFPTGYGSSSRRAPQTGIVDECCFRSCDLRRLEMYCAPLKPAKSARSV | 121 |
| | . . . | |
| 106 | RAQRHTDMPKTQKYQPPSTNKNTKSQRRKG | 135 |
| | \|\|\|\|\|\|\|\|\|\|\|\|\|\|\|\|\|\|\|\|\|\|\|\|\|\|\|\|\| | |
| 122 | RAQRHTDMPKTQKYQPPSTNKNTKSQRRKG | 151 |

Sequence name: Q14620 (SEQ ID NO:221)
Sequence documentation:
Alignment of: HSIGFACI_PEA_1_P5 (SEQ ID NO:225) ×
Q14620 (SEQ ID NO:221) . .
Alignment segment 1/1:

| | | | |
|---|---|---|---|
| Quality: | 1175.00 | Escore: | 0 |
| Matching length: | 118 | Total length: | 118 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MITPTVKMHTMSSSHLFYLALCLLTFTSSATAGPETLCGAELVDALQFVC | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MITPTVKMHTMSSSHLFYLALCLLTFTSSATAGPETLCGAELVDALQFVC | 50 |
| | . . . . . | |
| 51 | GDRGFYFNKPTGYGSSSRRAPQTGIVDECCFRSCDLRRLEMYCAPLKPAK | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | GDRGFYFNKPTGYGSSSRRAPQTGIVDECCFRSCDLRRLEMYCAPLKPAK | 100 |
| | . | |
| 101 | SARSVRAQRHTDMPKTQK | 118 |
| | \|\|\|\|\|\|\|\|\|\|\|\|\|\|\|\|\|\| | |
| 101 | SARSVRAQRHTDMPKTQK | 118 |

| Sequence name: IGFA_HUMAN (SEQ ID NO:223) | | | |
|---|---|---|---|
| Sequence documentation: | | | |
| Alignment of: HSIGFACI_PEA_1_P5 (SEQ ID NO:225) × | | | |
| IGFA_HUMAN (SEQ ID NO:223) . . | | | |
| Alignment segment 1/1: | | | |
| Quality: | 1125.00 | Escore: | 0 |
| Matching length: | 113 | Total length: | 113 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 22 | VKMHTMSSSHLFYLALCLLTFTSSATAGPETLCGAELVDALQFVCGDRGF | 71 |
| | . . . . . | |
| 56 | YFNKPTGYGSSSRRAPQTGIVDECCFRSCDLRRLEMYCAPLKPAKSARSV | 105 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 72 | YFNKPTGYGSSSRRAPQTGIVDECCFRSCDLRRLEMYCAPLKPAKSARSV | 121 |
| | . | |
| 106 | RAQRHTDMPKTQK | 118 |
| | \|\|\|\|\|\|\|\|\|\|\|\|\| | |
| 122 | RAQRHTDMPKTQK | 134 |

| Sequence name: IGFA_HUMAN (SEQ ID NO:223) | | | |
|---|---|---|---|
| Sequence documentation: | | | |
| Alignment of: HSIGFACI_PEA_1_P2 (SEQ ID NO:226) × | | | |
| IGFA_HUMAN (SEQ ID NO:223) . . | | | |
| Alignment segment 1/1: | | | |
| Quality: | 1313.00 | Escore: | 0 |
| Matching length: | 132 | Total length: | 132 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF 55 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 22 | VKMHTMSSSHLFYLALCLLTFTSSATAGPETLCGAELVDALQFVCGDRGF | 71 |
| | . . . . . | |
| 56 | YFNKPTGYGSSSRRAPQTGIVDECCFRSCDLRRLEMYCAPLKPAKSARSV | 105 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 72 | YFNKPTGYGSSSRRAPQTGIVDECCFRSCDLRRLEMYCAPLKPAKSARSV | 121 |
| | . . . | |
| 106 | RAQRHTDMPKTQKEVHLKNASRGSAGNKNYRM | 137 |
| | \|\|\|\|\|\|\|\|\|\|\|\|\|\|\|\|\|\|\|\|\|\|\|\|\|\|\|\|\|\|\|\| | |
| 122 | RAQRHTDMPKTQKEVHLKNASRGSAGNKNYRM | 153 |

| Sequence name: IGFA_HUMAN (SEQ ID NO:223) | | | |
|---|---|---|---|
| Sequence documentation: | | | |
| Alignment of: HSIGFACI_PEA_1_26 (SEQ ID NO: 227) × | | | |
| IGFA_HUMAN (SEQ ID NO:223) . . | | | |
| Alignment segment 1/1: | | | |
| Quality: | 1343.00 | Escore: | 0 |
| Matching length: | 134 | Total length: | 134 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MGKISSLPTQLFKCCFCDFLKVKMHTMSSSHLFYLALCLLTFTSSATAGP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MGKISSLPTQLFKCCFCDFLKVKMHTMSSSHLFYLALCLLTFTSSATAGP | 50 |
| | . . . . . | |
| 51 | ETLCGAELVDALQFVCGDRGFYFNKPTGYGSSSRRAPQTGIVDECCFRSC | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ETLCGAELVDALQFVCGDRGFYFNKPTGYGSSSRRAPQTGIVDECCFRSC | 100 |
| | . . . | |
| 101 | DLRRLEMYCAPLKPAKSARSVRAQRHTDMPKTQK | 134 |
| | \|\|\|\|\|\|\|\|\|\|\|\|\|\|\|\|\|\|\|\|\|\|\|\|\|\|\|\|\|\|\|\|\|\| | |
| 101 | DLRRLEMYCAPLKPAKSARSVRAQRHTDMPKTQK | 134 |

| Sequence name: IGFB_HUMAN (SEQ ID NO:220) | | | |
|---|---|---|---|
| Sequence documentation: | | | |
| Alignment of: HSIGFACI_PEA_1_P1 (SEQ ID NO:228) × | | | |
| IGFB HUMAN (SEQ ID NO:220) . . | | | |
| Alignment segment 1/1: | | | |
| Quality: | 1343.00 | Escore: | 0 |
| Matching length: | 134 | Total length: | 134 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MGKISSLPTQLFKCCFCDFLKVKMHTMSSSHLFYLALCLLTFTSSATAGP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MGKISSLPTQLFKCCFCDFLKVKMHTMSSSHLFYLALCLLTFTSSATAGP | 50 |
| | . . . . . | |
| 51 | ETLCGAELVDALQFVCGDRGFYFNKPTGYGSSSRRAPQTGIVDECCFRSC | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ETLCGAELVDALQFVCGDRGFYFNKPTGYGSSSRRAPQTGIVDECCFRSC | 100 |
| | . . . | |
| 101 | DLRRLEMYCAPLKPAKSARSVRAQRHTDMPKTQK | 134 |
| | \|\|\|\|\|\|\|\|\|\|\|\|\|\|\|\|\|\|\|\|\|\|\|\|\|\|\|\|\|\|\|\|\|\| | |
| 101 | DLRRLEMYCAPLKPAKSARSVRAQRHTDMPKTQK | 134 |

| Sequence name: IGFB_HUMAN (SEQ ID NO:220) | | | |
|---|---|---|---|
| Sequence documentation: | | | |
| Alignment of: HSIGFACI_PEA_1_P7 (SEQ ID NO:229) × | | | |
| IGFB HUMAN (SEQ ID NO:220) . . | | | |
| Alignment segment 1/1: | | | |
| Quality: | 729.00 | Escore: | 0 |
| Matching length: | 75 | Total length: | 75 |
| Matching Percent | 100.00 | Matching Percent | 97.33 |
| Similarity: | | Identity: | |
| Total Percent | 100.00 | Total Percent | 97.33 |
| Similarity: | | Identity: | |
| Gaps: | 0 | | |

| Alignment: | | |
|---|---|---|
| | . . . . . | |
| 1 | MGKISSLPTQLFKCCFCDFLKVKMHTMSSSHLFYLALCLLTFTSSATAGP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MGKISSLPTQLFKCCFCDFLKVKMHTMSSSHLFYLALCLLTFTSSATAGP | 50 |
| | . . | |
| 51 | ETLCGAELVDALQFVCGDRGFYFSR | 75 |
| | \|\|\|\|\|\|\|\|\|\|\|\|\|\|\|\|\|\|\|\|\|\|\|:: | |
| 51 | ETLCGAELVDALQFVCGDRGFYFNK | 75 |

| Sequence name: IGFA_HUMAN (SEQ ID NO:223) | | | |
|---|---|---|---|
| Sequence documentation: | | | |
| Alignment of: HSIGFACI_PEA_1_P7 (SEQ ID NO:229) × | | | |
| IGFA HUMAN (SEQ ID NO:223) . . | | | |
| Alignment segment 1/1: | | | |
| Quality: | 729.00 | Escore: | 0 |
| Matching length: | 75 | Total length: | 75 |
| Matching Percent | 100.00 | Matching Percent | 97.33 |
| Similarity: | | Identity: | |
| Total Percent | 100.00 | Total Percent | 97.33 |
| Similarity: | | Identity: | |
| Gaps: | 0 | | |

| Alignment: | | |
|---|---|---|
| | . . . . . | |
| 1 | MGKISSLPTQLFKCCFCDFLKVKMHTMSSSHLFYLALCLLTFTSSATAGP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MGKISSLPTQLFKCCFCDFLKVKMHTMSSSHLFYLALCLLTFTSSATAGP | 50 |
| | . . | |
| 51 | ETLCGAELVDALQFVCGDRGFYFSR | 75 |
| | \|\|\|\|\|\|\|\|\|\|\|\|\|\|\|\|\|\|\|\|\|\|\|\|\| | |
| 51 | ETLCGAELVDALQFVCGDRGFYFNK | 75 |

| Sequence name: Q9NP10 (SEQ ID NO:222) | | | |
|---|---|---|---|
| Sequence documentation: | | | |
| Alignment of: HSIGFACI_PEA_1_P8 (SEQ ID NO:230) × | | | |
| Q9NP10 (SEQ ID NO:222) | | | |
| Alignment segment 1/1: | | | |
| Quality: | 493.00 | Escore: | 0 |
| Matching length: | 52 | Total length: | 52 |
| Matching Percent | 100.00 | Matching Percent | 96.15 |
| Similarity: | | Identity: | |
| Total Percent | 100.00 | Total Percent | 96.15 |
| Similarity: | | Identity: | |
| Gaps: | 0 | | |

| Alignment: | | |
|---|---|---|
| | . . . . . | |
| 8 | MHTMSSSHLFYLALCLLTFTSSATAGPETLCGAELVDALQFVCGDRGFYF | 57 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MHTMSSSHLFYLALCLLTFTSSATAGPETLCGAELVDALQFVCGDRGFYF | 50 |
| 58 | YFSR | 59 |
| | \|\|:: | |
| 51 | YFNK | 52 |

| Sequence name: Q13429 (SEQ ID NQ:224) | | | |
|---|---|---|---|
| Sequence documentation: | | | |
| Alignment of: HSIGFACI_PEA_1_P8 (SEQ ID NO:230) × | | | |
| Q13429 (SEQ ID NO:224) . . | | | |
| Alignment segment 1/1: | | | |
| Quality: | 511.00 | Escore: | 0 |
| Matching length: | 54 | Total length: | 54 |
| Matching Percent | 100.00 | Matching Percent | 96.30 |
| Similarity: | | Identity: | |
| Total Percent | 100.00 | Total Percent | 96.30 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 3 | VKMHTMSSSHLFYLALCLLTFTSSATAGPETLCGAELVDALQFVCGDRGF | 52 |
| 56 | YFSR | 59 |
| | \|\|:: | |
| 53 | YFNK | 56 |

| Sequence name: Q14620 (SEQ ID NO:221) | | | |
|---|---|---|---|
| Sequence documentation: | | | |
| Alignment of: HSIGFACI_PEA_1_P8 (SEQ ID NO:230) × | | | |
| Q14620 (SEQ ID NO:221) . . | | | |
| Alignment segment 1/1: | | | |
| Quality: | 561.00 | Escore: | 0 |
| Matching length: | 59 | Total length: | 59 |
| Matching Percent | 100.00 | Matching Percent | 96.61 |
| Similarity: | | Identity: | |
| Total Percent | 100.00 | Total Percent | 96.61 |
| Similarity: | | Identity: | |
| Gaps: | 0 | | |

| Alignment: | | |
|---|---|---|
| | . . . . . | |
| 1 | MITPTVKMHTMSSSHLFYLALCLLTFTSSATAGPETLCGAELVDALQFVC | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MITPTVKMHTMSSSHLFYLALCLLTFTSSATAGPETLCGAELVDALQFVC | 50 |
| 51 | GDRGFYFSR | 59 |
| | \|\|\|\|\|\|\|:: | |
| 51 | GDRGFYFNK | 59 |

| Sequence name: IGFB_HUMAN (SEQ ID NO:220) | | | |
|---|---|---|---|
| Sequence documentation: | | | |
| Alignment of: HSIGFACI_PEA_1_P8 (SEQ ID NO:230) × | | | |
| IGFB_HUMAN (SEQ ID NO:220) . . | | | |
| Alignment segment 1/1: | | | |
| Quality: | 511.00 | Escore: | 0 |
| Matching length: | 54 | Total length: | 54 |
| Matching Percent | 100.00 | Matching Percent | 96.30 |
| Similarity: | | Identity: | |
| Total Percent | 100.00 | Total Percent | 96.30 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 22 | VKMHTMSSSHLFYLALCLLTFTSSATAGPETLCGAELVDALQFVCGDRGF | 71 |
| 56 | YFSR | 59 |
| | \|\|:: | |
| 72 | YFNK | 75 |

| Sequence name: IGFA_HUMAN (SEQ ID NO:223) | | | |
|---|---|---|---|
| Sequence documentation: | | | |
| Alignment of: HSIGFACI_PEA_1_P8 (SEQ ID NO:230) × | | | |
| IGFA HUMAN (SEQ ID NO:223) . . | | | |
| Alignment segment 1/1: | | | |
| Quality: | 511.00 | Escore: | 0 |
| Matching length: | 54 | Total length: | 54 |
| Matching Percent | 100.00 | Matching Percent | 96.30 |
| Similarity: | | Identity: | |
| Total Percent | 100.00 | Total Percent | 96.30 |
| Similarity: | | Identity: | |
| Gaps: | 0 | | |

| Alignment: | | |
|---|---|---|
| | . . . . . | |
| 6 | VKMHTMSSSHLFYLALCLLTFTSSATAGPETLCGAELVDALQFVCGDRGF | 55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 22 | VKMHTMSSSHLFYLALCLLTFTSSATAGPETLCGAELVDALQFVCGDRGF | 71 |
| 56 | YFSR | 59 |
| | \|\|:: | |
| 72 | YFNK | 75 |

### Description for Cluster HSSTROMR

Cluster HSSTROMR features 1 transcript(s) and 11 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HSSTROMR_PEA_1_T3 | 231 |

TABLE 2
Segments of interest

| Segment Name | Sequence ID No. |
|---|---|
| HSSTROMR_PEA_1_node_0 | 232 |
| HSSTROMR_PEA_1_node_5 | 233 |
| HSSTROMR_PEA_1_node_7 | 234 |
| HSSTROMR_PEA_1_node_9 | 235 |
| HSSTROMR_PEA_1_node_13 | 236 |
| HSSTROMR_PEA_1_node_16 | 237 |
| HSSTROMR_PEA_1_node_18 | 238 |
| HSSTROMR_PEA_1_node_20 | 239 |
| HSSTROMR_PEA_1_node_28 | 240 |
| HSSTROMR_PEA_1_node_14 | 241 |
| HSSTROMR_PEA_1_node_22 | 242 |

TABLE 3
Proteins of interest

| | Sequence | |
|---|---|---|
| Protein Name | ID No. | Corresponding Transcript(s) |
| HSSTROMR_PEA_1_P4 | 244 | HSSTROMR_PEA_1_T3 |
| | | (SEQ ID NO: 231) |

These sequences are variants of the known protein Stromelysin-1 precursor (SEQ ID NO:243) (SwissProt accession identifier MM03_HUMAN; known also according to the synonyms EC 3.4.24.17; Matrix metalloproteinase-3; MMP-3; Transin-1; SL-1), referred to herein as the previously known protein.

Protein Stromelysin-1 precursor (SEQ ID NO:243) is known or believed to have the following function(s): can degrade fibronectin, laminin, gelatins of type I, III, IV, and V; collagens III, IV, X, and IX, and cartilage proteoglycans. Activates procollagenase. The sequence for protein Stromelysin-1 precursor is given at the end of the application, as “Stromelysin-1 precursor amino acid sequence” (SEQ ID NO:243). Known polymorphisms for this sequence are as shown in Table 4.

TABLE 4
Amino acid mutations for Known Protein

| SNP position(s) on | |
|---|---|
| amino acid sequence | Comment |
| 45 | K -> E. /FTId = VAR_013090. |
| 420 | P -> L |

The following GO Annotation(s) apply to the previously known protein. The following annotation(s) were found: proteolysis and peptidolysis, which are annotation(s) related to Biological Process; stromelysin 1; calcium binding; zinc binding; hydrolase, which are annotation(s) related to Molecular Function; and extracellular matrix; extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

This protein was found to be upregulated in endometriosis (Yang et al, Best Pract Res Clin Obstet Gynaecol. 2004 April; 18(2):305-18). Variants of this cluster are suitable for use as diagnostic markers for endometriosis.

As noted above, cluster HSSTROMR features 1 transcript(s), which were listed in Table 1 above. These transcript(s) encode for protein(s) which are variant(s) of protein Stromelysin-1 precursor (SEQ ID NO:243). A description of each variant protein according to the present invention is now provided.

Variant protein HSSTROMR_PEA_{—}1_P4 (SEQ ID NO:244) according to the present invention has an amino acid sequence as given at the end of the application; it is encoded by transcript(s) HSSTROMR_PEA_{—}1_T3 (SEQ ID NO:231). An alignment is given to the known protein (Stromelysin-1 precursor (SEQ ID NO:243)) at the end of the application. One or more alignments to one or more previously published protein sequences are given at the end of the application. A brief description of the relationship of the variant protein according to the present invention to each such aligned protein is as follows:

Comparison report between HSSTROMR_PEA_{—}1_P4 (SEQ ID NO:244) and MM03_HUMAN (SEQ ID NO:243):

1. An isolated chimeric polypeptide encoding for HSSTROMR_PEA_{—}1_P4 (SEQ ID NO:244), comprising a first amino acid sequence being at least 90% homologous to MKSLPILLLLCVAVCSAYPLDGAARGEDTSMNLV corresponding to amino acids 1-34 of MM03_HUMAN (SEQ ID NO:243), which also corresponds to amino acids 1-34 of HSSTROMR_PEA_{—}1_P4 (SEQ ID NO:244), and a second amino acid sequence being at least 90% homologous to QKFLGLEVTGKLDSDTLEVMRKPRCGVPDVGHFRTFPGIPKWRKTHLTYRIVNYTPDLP KDAVDSAVEKALKVWEEVTPLTFSRLYEGEADIMISFAVREHGDFYPFDGPGNVLAHA YAPGPGINGDAHFDDDEQWTKDTTGTNLFLVAAHEIGHSLGLFHSANTEALMYPLYHS LTDLTRFRLSQDDINGIQSLYGPPPDSPETPLVPTEPVPPEPGTPANCDPALSFDAVSTLR GEILIFKDRHFWRKSLRKLEPELHLISSFWPSLPSGVDAAYEVTSKDLVFIFKGNQFWAIR GNEVRAGYPRGIHTLGFPPTVRKIDAAISDKEKNKTYFFVEDKYWRFDEKRNSMEPGFP KQIAEDFPGIDSKIDAVFEEFGFFYFFTGSSQLEFDPNAKKVTHTLKSNSWLNC corresponding to amino acids 68-477 of MM03_HUMAN (SEQ ID NO:243), which also corresponds to amino acids 35-444 of HSSTROMR_PEA_{—}1_P4 (SEQ ID NO:244), wherein said first amino acid sequence and second amino acid sequence are contiguous and in a sequential order.

2. An isolated chimeric polypeptide encoding for an edge portion of HSSTROMR_PEA_{—}1_P4 (SEQ ID NO:244), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VQ, having a structure as follows: a sequence starting from any of amino acid numbers 34−x to 34; and ending at any of amino acid numbers 35+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROMR_PEA_{—}1_P4 (SEQ ID NO:244) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein HSSTROMR_PEA_{—}1_P4 (SEQ ID NO:244) Sequence provides support for the deduced sequence of this variant protein according to the present invention).

TABLE 5
Amino acid mutations

| SNP position(s) on | Alternative | Previously |
|---|---|---|
| amino acid sequence | amino acid(s) | known SNP? |
| 29 | T -> N | No |
| 38 | L -> P | No |
| 38 | L -> | No |
| 48 | S -> F | No |
| 56 | K -> | No |
| 56 | K -> N | No |
| 80 | H -> P | Yes |
| 147 | V -> | No |
| 254 | P -> A | No |
| 366 | K -> | No |
| 413 | F -> L | No |
| 413 | F -> V | No |
| 427 | P -> | No |

The glycosylation sites of variant protein HSSTROMR_PEA_{—}1_P4 (SEQ ID NO:244), as compared to the known protein Stromelysin-1 precursor (SEQ ID NO:243), are described in Table 6 (given according to their position(s) on the amino acid sequence in the first column; the second column indicates whether the glycosylation site is present in the variant protein; and the last column indicates whether the position is different on the variant protein).

TABLE 6
Glycosylation site(s)

| Position(s) on known | Present in variant | Position in variant |
|---|---|---|
| amino acid sequence | protein? | protein? |
| 120 | yes | 87 |

Variant protein HSSTROMR_PEA_{—}1_P4 (SEQ ID NO:244) is encoded by the following transcript(s): HSSTROMR_PEA_{—}1_T3 (SEQ ID NO:231), for which the sequence(s) is/are given at the end of the application. The coding portion of transcript HSSTROMR_PEA_{—}1_T3 (SEQ ID NO:231) is shown in bold; this coding portion starts at position 70 and ends at position 1401. The transcript also has the following SNPs as listed in Table 7 (given according to their position on the nucleotide sequence, with the alternative nucleic acid listed; the last column indicates whether the SNP is known or not; the presence of known SNPs in variant protein HSSTROMR_PEA_{—}1_P4 (SEQ ID NO:244) Sequence provides support for the deduced sequence of this variant protein according to the present invention).

TABLE 7
Nucleic acid SNPs

| SNP position on nucleotide | Alternative | Previously known |
|---|---|---|
| sequence | nucleic acid | SNP? |
| 49 | A -> G | No |
| 155 | C -> A | No |
| 182 | T -> | No |
| 182 | T -> C | No |
| 212 | C -> T | No |
| 237 | G -> | No |
| 237 | G -> C | No |
| 258 | C -> T | Yes |
| 276 | C -> G | No |
| 308 | A -> C | Yes |
| 509 | T -> | No |
| 762 | A -> G | Yes |
| 829 | C -> G | No |
| 1056 | C -> T | Yes |
| 1165 | A -> | No |
| 1306 | T -> C | No |
| 1306 | T -> G | No |
| 1350 | A -> | No |
| 1425 | A -> | No |
| 1437 | T -> | No |
| 1518 | C -> T | Yes |
| 1538 | C -> T | Yes |
| 1557 | G -> A | Yes |

As noted above, cluster HSSTROMR features 11 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSSTROMR_PEA_{—}1_node_{—}0 (SEQ ID NO:232) according to the present invention is supported by 39 libraries. The number of libraries was determined as previously described. This segment can be found in the following transcript(s): HSSTROMR_PEA_{—}1_T3 (SEQ ID NO:231). Table 8 below describes the starting and ending position of this segment on each transcript.

TABLE 8
Segment location on transcripts

| | Segment | Segment |
|---|---|---|
| Transcript name | starting position | ending position |
| HSSTROMR_PEA_1_T3 (SEQ ID | 1 | 174 |
| NO: 231) | | |

Segment cluster HSSTROMR_PEA_{—}1_node_{—}5 (SEQ ID NO:233) according to the present invention is supported by 45 libraries. The number of libraries was determined as previously described. This segment can be found in the following transcript(s): HSSTROMR_PEA_{—}1_T3 (SEQ ID NO:231). Table 9 below describes the starting and ending position of this segment on each transcript.

TABLE 9
Segment location on transcripts

| | Segment | Segment |
|---|---|---|
| Transcript name | starting position | ending position |
| HSSTROMR_PEA_1_T3 (SEQ ID | 175 | 320 |
| NO: 231) | | |

Segment cluster HSSTROMR_PEA_{—}1_node_{—}7 (SEQ ID NO:234) according to the present invention is supported by 41 libraries. The number of libraries was determined as previously described. This segment can be found in the following transcript(s): HSSTROMR_PEA_{—}1_T3 (SEQ ID NO:231). Table 10 below describes the starting and ending position of this segment on each transcript.

TABLE 10
Segment location on transcripts

| | Segment | Segment |
|---|---|---|
| Transcript name | starting position | ending position |
| HSSTROMR_PEA_1_T3 (SEQ ID | 321 | 469 |
| NO: 231) | | |

Segment cluster HSSTROMR_PEA_{—}1_node_{—}9 (SEQ ID NO:235) according to the present invention is supported by 40 libraries. The number of libraries was determined as previously described. This segment can be found in the following transcript(s): HSSTROMR_PEA_{—}1_T3 (SEQ ID NO:231). Table 11 below describes the starting and ending position of this segment on each transcript.

TABLE 11
Segment location on transcripts

| | Segment | Segment |
|---|---|---|
| Transcript name | starting position | ending position |
| HSSTROMR_PEA_1_T3 (SEQ ID | 470 | 595 |
| NO: 231) | | |

Segment cluster HSSTROMR_PEA_{—}1_node_{—}13 (SEQ ID NO:236) according to the present invention is supported by 46 libraries. The number of libraries was determined as previously described. This segment can be found in the following transcript(s): HSSTROMR_PEA_{—}1_T3 (SEQ ID NO:231). Table 12 below describes the starting and ending position of this segment on each transcript.

TABLE 12
Segment location on transcripts

| | Segment | Segment |
|---|---|---|
| Transcript name | starting position | ending position |
| HSSTROMR_PEA_1_T3 (SEQ ID | 596 | 730 |
| NO: 231) | | |

Segment cluster HSSTROMR_PEA_{—}1_node_{—}16 (SEQ ID NO:237) according to the present invention is supported by 43 libraries. The number of libraries was determined as previously described. This segment can be found in the following transcript(s): HSSTROMR_PEA_{—}1_T3 (SEQ ID NO:231). Table 13 below describes the starting and ending position of this segment on each transcript.

TABLE 13
Segment location on transcripts

| | Segment | Segment |
|---|---|---|
| Transcript name | starting position | ending position |
| HSSTROMR_PEA_1_T3 (SEQ ID | 761 | 905 |
| NO: 231) | | |

Segment cluster HSSTROMR_PEA_{—}1_node_{—}18 (SEQ ID NO:238) according to the present invention is supported by 45 libraries. The number of libraries was determined as previously described. This segment can be found in the following transcript(s): HSSTROMR_PEA_{—}1_T3 (SEQ ID NO:231). Table 14 below describes the starting and ending position of this segment on each transcript.

TABLE 14
Segment location on transcripts

| | Segment | Segment |
|---|---|---|
| Transcript name | starting position | ending position |
| HSSTROMR_PEA_1_T3 (SEQ ID | 906 | 1039 |
| NO: 231) | | |

Segment cluster HSSTROMR_PEA_{—}1_node_{—}20 (SEQ ID NO:239) according to the present invention is supported by 57 libraries. The number of libraries was determined as previously described. This segment can be found in the following transcript(s): HSSTROMR_PEA_{—}1_T3 (SEQ ID NO:231). Table 15 below describes the starting and ending position of this segment on each transcript.

TABLE 15
Segment location on transcripts

| | Segment | Segment |
|---|---|---|
| Transcript name | starting position | ending position |
| HSSTROMR_PEA_1_T3 (SEQ ID | 1040 | 1199 |
| NO: 231) | | |

Segment cluster HSSTROMR_PEA_{—}1_node_{—}28 (SEQ ID NO:240) according to the present invention is supported by 66 libraries. The number of libraries was determined as previously described. This segment can be found in the following transcript(s): HSSTROMR_PEA_{—}1_T3 (SEQ ID NO:231). Table 16 below describes the starting and ending position of this segment on each transcript.

TABLE 16
Segment location on transcripts

| | Segment | Segment |
|---|---|---|
| Transcript name | starting position | ending position |
| HSSTROMR_PEA_1_T3 (SEQ ID | 1304 | 1738 |
| NO: 231) | | |

According to an optional embodiment of the present invention, short segments related to the above cluster are also provided. These segments are up to about 120 bp in length, and so are included in a separate description.

Segment cluster HSSTROMR_PEA_{—}1_node_{—}14 (SEQ ID NO:241) according to the present invention is supported by 42 libraries. The number of libraries was determined as previously described. This segment can be found in the following transcript(s): HSSTROMR_PEA_{—}1_T3 (SEQ ID NO:231). Table 17 below describes the starting and ending position of this segment on each transcript.

TABLE 17
Segment location on transcripts

| | Segment | Segment |
|---|---|---|
| Transcript name | starting position | ending position |
| HSSTROMR_PEA_1_T3 (SEQ ID | 731 | 760 |
| NO: 231) | | |

Segment cluster HSSTROMR_PEA_{—}1_node_{—}22 (SEQ ID NO:242) according to the present invention is supported by 58 libraries. The number of libraries was determined as previously described. This segment can be found in the following transcript(s): HSSTROMR_PEA_{—}1_T3 (SEQ ID NO:231). Table 18 below describes the starting and ending position of this segment on each transcript.

TABLE 18
Segment location on transcripts

| | Segment | Segment |
|---|---|---|
| Transcript name | starting position | ending position |
| HSSTROMR_PEA_1_T3 (SEQ ID | 1200 | 1303 |
| NO: 231) | | |

Variant protein alignment to the previously known protein: Sequence name: MM03_HUMAN (SEQ ID NO:243)

| Sequence name: MN03_HUMAN (SEQ ID NO:243) | | | |
|---|---|---|---|
| Sequence documentation: | | | |
| Alignment of: HSSTROMR_PEA_1_P4 (SEQ ID NO:244) × | | | |
| MN03_HUMAN (SEQ ID NO:243) . . | | | |
| Alignment segment 1/1: | | | |
| Quality: | 4302.00 | Escore: | 0 |
| Matching length: | 444 | Total length: | 477 |
| Matching Percent | 100.00 | Matching Percent | 100.00 |
| Similarity: | | Identity: | |
| Total Percent | 93.08 | Total Percent | 93.08 |
| Similarity: | | Identity: | |
| Gaps: | 1 | | |

| Alignment: | | |
|---|---|---|
| | . . . . . | |
| 1 | MKSLPILLLLCVAVCSAYPLDGAARGEDTSMNLV................ | 34 |
| | \|\|\|\|\|\|\|\|\|\|\|\|\|\|\|\|\|\|\|\|\|\|\|\|\|\|\|\|\|\|\|\|\|\| | |
| 1 | MKSLPILLLLCVAVCSAYPLDGAARGEDTSMNLVQKYLENYYDLKKDVKQ | 50 |
| | . . . . . | |
| 35 | .................QKFLGLEVTGKLDSDTLEVMRKPRCGVPDVGHF | 67 |
| | \|\|\|\|\|\|\|\|\|\|\|\|\|\|\|\|\|\|\|\|\|\|\|\|\|\|\|\|\|\|\|\|\| | |
| 51 | FVRRKDSGPVVKKIREMQKFLGLEVTGKLDSDTLEVMRKPRCGVPDVGHF | 100 |
| | . . . . . | |
| 68 | RTFPGIPKWRKTHLTYRIVNYTPDLPKDAVDSAVEKALKVWEEVTPLTFS | 117 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | RTFPGIPKWRKTHLTYRIVNYTPDLPKDAVDSAVEKALKVWEEVTPLTFS | 150 |
| | . . . . . | |
| 118 | RLYEGEADIMISFAVREHGDFYPFDGPGNVLAHAYAPGPGINGDAHFDDD | 167 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | RLYEGEADIMISFAVREHGDFYPFDGPGNVLAHAYAPGPGINGDAHFDDD | 200 |
| | . . . . . | |
| 168 | EQWTKDTTGTNLFLVAAHEIGHSLGLFHSANTEALMYPLYHSLTDLTRFR | 217 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | EQWTKDTTGTNLFLVAAHEIGHSLGLFHSANTEALMYPLYHSLTDLTRFR | 250 |
| | . . . . . | |
| 218 | LSQDDINGIQSLYGPPPDSPETPLVPTEPVPPEPGTPANCDPALSFDAVS | 267 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | LSQDDINGIQSLYGPPPDSPETPLVPTEPVPPEPGTPANCDPALSFDAVS | 300 |
| | . . . . . | |
| 268 | TLRGEILIFKDRHFWRKSLRKLEPELHLISSFWPSLPSGVDAAYEVTSKD | 317 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | TLRGEILIFKDRHFWRKSLRKLEPELHLISSFWPSLPSGVDAAYEVTSKD | 350 |
| | . . . . . | |
| 318 | LVFIFKGNQFWAIRGNEVRAGYPRGIHTLGFPPTVRKIDAAISDKEKNKT | 367 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | LVFIFKGNQFWAIRGNEVRAGYPRGIHTLGFPPTVRKIDAAISDKEKNKT | 400 |
| | . . . . . | |
| 368 | YFFVEDKYWRFDEKRNSMEPGFPKQIAEDFFGIDSKIDAVFEEFGFFYFF | 417 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | YFFVEDKYWRFDEKRNSMEPGFPKQIAEDFPGIDSKIDAVFEEFGFFYFF | 450 |
| | . . | |
| 418 | TGSSQLEFDPNAKKVTHTLKSNSWLNC | 444 |
| | \|\|\|\|\|\|\|\|\|\|\|\|\|\|\|\|\|\|\|\|\|\|\|\|\|\|\| | |
| 451 | TGSSQLEFDPNAKKVTHTLKSNSWLNC | 477 |

### Description for Cluster HUM4COLA

Cluster HUM4COLA features 3 transcript(s) and 27 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HUM4COLA_PEA_1_T1 | 245 |
| HUM4COLA_PEA_1_T5 | 246 |
| HUM4COLA_PEA_1_T6 | 247 |

TABLE 2
Segments of interest

| Segment Name | Sequence ID No. |
|---|---|
| HUM4COLA_PEA_1_node_0 | 248 |
| HUM4COLA_PEA_1_node_2 | 249 |
| HUM4COLA_PEA_1_node_4 | 250 |
| HUM4COLA_PEA_1_node_7 | 251 |
| HUM4COLA_PEA_1_node_11 | 252 |
| HUM4COLA_PEA_1_node_19 | 253 |
| HUM4COLA_PEA_1_node_40 | 254 |
| HUM4COLA_PEA_1_node_41 | 255 |
| HUM4COLA_PEA_1_node_8 | 256 |
| HUM4COLA_PEA_1_node_9 | 257 |
| HUM4COLA_PEA_1_node_10 | 258 |
| HUM4COLA_PEA_1_node_12 | 259 |
| HUM4COLA_PEA_1_node_13 | 260 |
| HUM4COLA_PEA_1_node_16 | 261 |
| HUM4COLA_PEA_1_node_17 | 262 |
| HUM4COLA_PEA_1_node_22 | 263 |
| HUM4COLA_PEA_1_node_23 | 264 |
| HUM4COLA_PEA_1_node_24 | 265 |
| HUM4COLA_PEA_1_node_25 | 266 |
| HUM4COLA_PEA_1_node_26 | 267 |
| HUM4COLA_PEA_1_node_27 | 268 |
| HUM4COLA_PEA_1_node_29 | 269 |
| HUM4COLA_PEA_1_node_30 | 270 |
| HUM4COLA_PEA_1_node_32 | 271 |
| HUM4COLA_PEA_1_node_33 | 272 |
| HUM4COLA_PEA_1_node_36 | 273 |
| HUM4COLA_PEA_1_node_37 | 274 |

TABLE 3
Proteins of interest

| | Sequence ID | |
|---|---|---|
| Protein Name | No. | Corresponding Transcript(s) |
| HUM4COLA_PEA_1_P7 | 276 | HUM4COLA_PEA_1_T6 |
| | | (SEQ ID NO: 247) |
| HUM4COLA_PEA_1_P14 | 277 | HUM4COLA_PEA_1_T1 |
| | | (SEQ ID NO: 245) |
| HUM4COLA_PEA_1_P15 | 278 | HUM4COLA_PEA_1_T5 |
| | | (SEQ ID NO: 246) |

These sequences are variants of the known protein 92 kDa type IV collagenase precursor (SEQ ID NO:275) (SwissProt accession identifier MM09_HUMAN; known also according to the synonyms EC 3.4.24.35; 92 kDa gelatinase; Matrix metalloproteinase-9; MMP-9; Gelatinase B; GELB), referred to herein as the previously known protein.

Protein 92 kDa type IV collagenase precursor (SEQ ID NO:275) is known or believed to have the following function(s): could play a role in bone osteoclastic resorption. The sequence for protein 92 kDa type IV collagenase precursor is given at the end of the application, as “92 kDa type IV collagenase precursor amino acid sequence” (SEQ ID NO:275). Known polymorphisms for this sequence are as shown in Table 4.

TABLE 4
Amino acid mutations for Known Protein

| SNP position(s) | |
|---|---|
| on amino acid | |
| sequence | Comment |
| 20 | A -> V (in dbSNP: 1805088). /FTId = VAR_013780. |
| 82 | E -> K (in dbSNP: 1805089). /FTId = VAR_013781. |
| 279 | R -> Q (common polymorphism; dbSNP: 17576). |
| | /FTId = VAR_013782. |
| 668 | R -> Q (in dbSNP: 17577). /FTId = VAR_014742. |
| 574 | P -> R |

The previously known protein also has the following indication(s) and/or potential therapeutic use(s): Peyronie's disease; Burns; Glaucoma; Wound healing; Ulcer; Dupuytren's disease. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Collagenase stimulant; Metalloproteinase-9 inhibitor; Microbial collagenase inhibitor; T cell stimulant. A therapeutic role for a protein represented by the cluster has been predicted. The cluster was assigned this field because there was information in the drug database or the public databases (e.g., described herein above) that this protein, or part thereof, is used or can be used for a potential therapeutic indication: Urological; Anticancer; Vulnerary; Musculoskeletal; Antiglaucoma; Neurological; Anti-inflammatory; Diagnostic; Monoclonal antibody, murine.

The following GO Annotation(s) apply to the previously known protein. The following annotation(s) were found: proteolysis and peptidolysis, which are annotation(s) related to Biological Process; gelatinase B; collagenase; zinc binding; hydrolase, which are annotation(s) related to Molecular Function; and extracellular matrix; extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

For the known protein, mRNA expression in endometriosis was higher than in normal endometrium (Ueda et al, Gynecol Endocrinol. 2002 October; 16(5):391-402). Variants of this cluster are suitable as diagnostic markers for endometriosis.

As noted above, cluster HUM4COLA features 3 transcript(s), which were listed in Table 1 above. These transcript(s) encode for protein(s) which are variant(s) of protein 92 kDa type IV collagenase precursor (SEQ ID NO:275). A description of each variant protein according to the present invention is now provided.

Variant protein HUM4COLA_PEA_{—}1_P7 (SEQ ID NO:276) according to the present invention has an amino acid sequence as given at the end of the application; it is encoded by transcript(s) HUM4COLA_PEA_{—}1_T6 (SEQ ID NO:247). An alignment is given to the known protein (92 kDa type IV collagenase precursor (SEQ ID NO:275)) at the end of the application. One or more alignments to one or more previously published protein sequences are given at the end of the application. A brief description of the relationship of the variant protein according to the present invention to each such aligned protein is as follows:

Comparison report between HUM4COLA_PEA_{—}1_P7 (SEQ ID NO:276) and MM09_HUMAN (SEQ ID NO:275):

1. An isolated chimeric polypeptide encoding for HUM4COLA_PEA_{—}1_P7 (SEQ ID NO:276), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGVVVPTRFGNADGAACHF PFIFEGRSYSACTTDGRSDGLPWCSTTANYDTDDRFGFCPSERLYTRDGNADGKPCQFP FIFQGQSYSACTTDGRSDGYRWCATTANYDRDKLFGFCPTRADSTVMGGNSAGELCVF PFTFLGKE corresponding to amino acids 1-357 of MM09_HUMAN (SEQ ID NO:275), which also corresponds to amino acids 1-357 of HUM4COLA_PEA_{—}1_P7 (SEQ ID NO:276), and a second amino acid sequence being at least 70%, optionally at least 80%, preferably at least 85%, more preferably at least 90% and most preferably at least 95% homologous to a polypeptide having the sequence SSP (SEQ ID NO:481) corresponding to amino acids 358-360 of HUM4COLA_PEA_{—}1_P7 (SEQ ID NO:276), wherein said first amino acid sequence and second amino acid sequence are contiguous and in a sequential order.

2. An isolated polypeptide encoding for a tail of HUM4COLA PEA_{—}1_P7 (SEQ ID NO:276), comprising a polypeptide being at least 70%, optionally at least about 80%, preferably at least about 85%, more preferably at least about 90% and most preferably at least about 95% homologous to the sequence SSP (SEQ ID NO:481) in HUM4COLA_PEA_{—}1_P7 (SEQ ID NO:276).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUM4COLA_PEA_{—}1_P7 (SEQ ID NO:276) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUM4COLA_PEA_{—}1_P7 (SEQ ID NO:276) Sequence provides support for the deduced sequence of this variant protein according to the present invention).

TABLE 7
Amino acid mutations

| SNP position(s) on amino acid | Alternative amino | Previously known |
|---|---|---|
| sequence | acid(s) | SNP? |
| 6 | P -> | No |
| 20 | A -> | No |
| 20 | A -> V | Yes |
| 65 | K -> E | No |
| 82 | E -> G | No |
| 82 | E -> K | Yes |
| 113 | D -> | No |
| 127 | N -> K | Yes |
| 160 | Y -> * | Yes |
| 165 | D -> N | Yes |
| 170 | F -> | No |
| 174 | E -> | No |
| 190 | H -> R | No |
| 205 | D -> G | No |
| 222 | F -> L | No |
| 229 | A -> | No |
| 237 | E -> | No |
| 255 | W -> R | No |
| 279 | R -> Q | Yes |
| 296 | G -> | No |
| 306 | G -> | No |
| 313 | W -> | No |

The glycosylation sites of variant protein HUM4COLA_PEA_{—}1_P7 (SEQ ID NO:276), as compared to the known protein 92 kDa type IV collagenase precursor (SEQ ID NO:275), are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on known amino | Present in variant | Position in variant |
|---|---|---|
| acid sequence | protein? | protein? |
| 38 | yes | 38 |
| 127 | yes | 127 |
| 120 | yes | 120 |

Variant protein HUM4COLA_PEA_{—}1_P7 (SEQ ID NO:276) is encoded by the following transcript(s): HUM4COLA_PEA_{—}1_T6 (SEQ ID NO:247), for which the sequence(s) is/are given at the end of the application. The coding portion of transcript HUM4COLA_PEA_{—}1_T6 (SEQ ID NO:247) is shown in bold; this coding portion starts at position 33 and ends at position 1112. The transcript also has the following SNPs as listed in Table 9 (given according to their position on the nucleotide sequence, with the alternative nucleic acid listed; the last column indicates whether the SNP is known or not; the presence of known SNPs in variant protein HUM4COLA_PEA_{—}1_P7 (SEQ ID NO:276) Sequence provides support for the deduced sequence of this variant protein according to the present invention).

TABLE 9
Nucleic acid SNPs

| SNP position on nucleotide | Alternative nucleic | Previously known |
|---|---|---|
| sequence | acid | SNP? |
| 48 | C -> | No |
| 91 | C -> | No |
| 91 | C -> T | Yes |
| 225 | A -> G | No |
| 276 | G -> A | Yes |
| 277 | A -> G | No |
| 371 | C -> | No |
| 413 | C -> G | Yes |
| 503 | T -> C | No |
| 512 | C -> G | Yes |
| 525 | G -> A | Yes |
| 540 | T -> | No |
| 554 | G -> | No |
| 601 | A -> G | No |
| 646 | A -> G | No |
| 698 | T -> G | No |
| 713 | -> A | No |
| 713 | -> T | No |
| 719 | C -> | No |
| 743 | G -> | No |
| 795 | T -> C | No |
| 868 | G -> A | Yes |
| 918 | G -> | No |
| 948 | G -> | No |
| 970 | G -> | No |
| 1112 | C -> T | Yes |
| 1118 | C -> T | Yes |
| 1409 | G -> | No |
| 1493 | C -> G | Yes |
| 1527 | C -> A | No |
| 1566 | G -> A | No |
| 1593 | A -> C | Yes |
| 1608 | G -> A | Yes |
| 1634 | G -> A | Yes |
| 1716 | C -> A | No |
| 1717 | G -> A | No |
| 1775 | G -> A | Yes |
| 1794 | C -> T | Yes |
| 1854 | G -> A | Yes |
| 1899 | C -> T | Yes |
| 1914 | G -> | No |
| 1935 | A -> C | Yes |
| 1952 | G -> A | Yes |
| 1992 | C -> T | Yes |
| 2042 | T -> C | Yes |
| 2086 | T -> | No |
| 2086 | T -> C | No |
| 2087 | T -> A | No |
| 2087 | T -> C | No |

Variant protein HUM4COLA_PEA_{—}1_P14 (SEQ ID NO:277) according to the present invention has an amino acid sequence as given at the end of the application; it is encoded by transcript(s) HUM4COLA_PEA_{—}1_T1 (SEQ ID NO:245). An alignment is given to the known protein (92 kDa type IV collagenase precursor (SEQ ID NO:275)) at the end of the application. One or more alignments to one or more previously published protein sequences are given at the end of the application. A brief description of the relationship of the variant protein according to the present invention to each such aligned protein is as follows:

Comparison report between HUM4COLA_PEA_{—}1_P14 (SEQ ID NO:277) and MM09_HUMAN (SEQ ID NO:275):

1. An isolated chimeric polypeptide encoding for HUM4COLA_PEA_{—}1_P14 (SEQ ID NO:277), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGVVVPTRFGNADGAACHF PFIFEGRSYSACTTDGRSDGLPWCSTTANYDTDDRFGFCPSE corresponding to amino acids 1-274 of MM09_HUMAN (SEQ ID NO:275), which also corresponds to amino acids 1-274 of HUM4COLA_PEA_{—}1_P14 (SEQ ID NO:277), and a second amino acid sequence being at least 70%, optionally at least 80%, preferably at least 85%, more preferably at least 90% and most preferably at least 95% homologous to a polypeptide having the sequence SE corresponding to amino acids 275-276 of HUM4COLA_PEA_{—}1_P14 (SEQ ID NO:277), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUM4COLA_PEA_{—}1_P14 (SEQ ID NO:277) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HUM4COLA_PEA_{—}1_P14 (SEQ ID NO:277) Sequence provides support for the deduced sequence of this variant protein according to the present invention).

TABLE 10
Amino acid mutations

| SNP position(s) on amino acid | Alternative amino | Previously known |
|---|---|---|
| sequence | acid(s) | SNP? |
| 6 | P -> | No |
| 20 | A -> | No |
| 20 | A -> V | Yes |
| 65 | K -> E | No |
| 82 | E -> G | No |
| 82 | E -> K | Yes |
| 113 | D -> | No |
| 127 | N -> K | Yes |
| 160 | Y -> * | Yes |
| 165 | D -> N | Yes |
| 170 | F -> | No |
| 174 | E -> | No |
| 190 | H -> R | No |
| 205 | D -> G | No |
| 222 | F -> L | No |
| 229 | A -> | No |
| 237 | E -> | No |
| 255 | W -> R | No |

The glycosylation sites of variant protein HUM4COLA_PEA_{—}1_P14 (SEQ ID NO:277), as compared to the known protein 92 kDa type IV collagenase precursor (SEQ ID NO:275), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | Present in variant | Position in variant |
|---|---|---|
| acid sequence | protein? | protein? |
| 38 | yes | 38 |
| 127 | yes | 127 |
| 120 | yes | 120 |

Variant protein HUM4COLA_PEA_{—}1_P14 (SEQ ID NO:277) is encoded by the following transcript(s): HUM4COLA_PEA_{—}1_T1 (SEQ ID NO:245), for which the sequence(s) is/are given at the end of the application. The coding portion of transcript HUM4COLA_PEA_{—}1_T1 (SEQ ID NO:245) is shown in bold; this coding portion starts at position 33 and ends at position 860. The transcript also has the following SNPs as listed in Table 12 (given according to their position on the nucleotide sequence, with the alternative nucleic acid listed; the last column indicates whether the SNP is known or not; the presence of known SNPs in variant protein HUM4COLA_PEA_{—}1_P14 (SEQ ID NO:277) Sequence provides support for the deduced sequence of this variant protein according to the present invention).

TABLE 12
Nucleic acid SNPs

| SNP position on nucleotide | Alternative nucleic | Previously known |
|---|---|---|
| sequence | acid | SNP? |
| 48 | C -> | No |
| 91 | C -> | No |
| 91 | C -> T | Yes |
| 225 | A -> G | No |
| 276 | G -> A | Yes |
| 277 | A -> G | No |
| 371 | C -> | No |
| 413 | C -> G | Yes |
| 503 | T -> C | No |
| 512 | C -> G | Yes |
| 525 | G -> A | Yes |
| 540 | T -> | No |
| 554 | G -> | No |
| 601 | A -> G | No |
| 646 | A -> G | No |
| 698 | T -> G | No |
| 713 | -> A | No |
| 713 | -> T | No |
| 719 | C -> | No |
| 743 | G -> | No |
| 795 | T -> C | No |
| 951 | -> A | No |
| 1125 | G -> A | Yes |
| 1175 | G -> | No |
| 1205 | G -> | No |
| 1227 | G -> | No |
| 1539 | C -> | No |
| 1629 | C -> T | Yes |
| 1635 | C -> T | Yes |
| 1926 | G -> | No |
| 2010 | C -> G | Yes |
| 2044 | C -> A | No |
| 2083 | G -> A | No |
| 2110 | A -> C | Yes |
| 2125 | G -> A | Yes |
| 2151 | G -> A | Yes |
| 2233 | C -> A | No |
| 2234 | G -> A | No |
| 2292 | G -> A | Yes |
| 2311 | C -> T | Yes |
| 2371 | G -> A | Yes |
| 2416 | C -> T | Yes |
| 2431 | G -> | No |
| 2452 | A -> C | Yes |
| 2469 | G -> A | Yes |
| 2509 | C -> T | Yes |
| 2559 | T -> C | Yes |
| 2603 | T -> | No |
| 2603 | T -> C | No |
| 2604 | T -> A | No |
| 2604 | T -> C | No |

Variant protein HUM4COLA_PEA_{—}1_P15 (SEQ ID NO:278) according to the present invention has an amino acid sequence as given at the end of the application; it is encoded by transcript(s) HUM4COLA_PEA_{—}1_T5 (SEQ ID NO:246). An alignment is given to the known protein (92 kDa type IV collagenase precursor (SEQ ID NO:275)) at the end of the application. One or more alignments to one or more previously published protein sequences are given at the end of the application. A brief description of the relationship of the variant protein according to the present invention to each such aligned protein is as follows:

Comparison report between HUM4COLA_PEA_{—}1_P15 (SEQ ID NO:278) and MM09_HUMAN:

1. An isolated chimeric polypeptide encoding for HUM4COLA_PEA_{—}1_P15 (SEQ ID NO:278), comprising a first amino acid sequence being at least 90% homologous to MSLWQPLVLVLLVLGCCFAAPRQRQSTLVLFPGDLRTNLTDRQLAEEYLYRYGYTRVA EMRGESKSLGPALLLLQKQLSLPETGELDSATLKAMRTPRCGVPDLGRFQTFEGDLKW HHHNITYWIQNYSEDLPRAVIDDAFARAFALWSAVTPLTFTRVYSRDADIVIQFGVAEH GDGYPFDGKDGLLAHAFPPGPGIQGDAHFDDDELWSLGKGV corresponding to amino acids 1-216 of MM09_HUMAN (SEQ ID NO:275), which also corresponds to amino acids 1-216 of HUM4COLA_PEA_{—}1_P15 (SEQ ID NO:278), and a second amino acid sequence being at least 70%, optionally at least 80%, preferably at least 85%, more preferably at least 90% and most preferably at least 95% homologous to a polypeptide having the sequence GEILSPPGP (SEQ ID NO:482) corresponding to amino acids 217-225 of HUM4COLA_PEA_{—}1_P15 (SEQ ID NO:278), wherein said first amino acid sequence and second amino acid sequence are contiguous and in a sequential order.

2. An isolated polypeptide encoding for a tail of HUM4COLA_PEA_{—}1_P15 (SEQ ID NO:278), comprising a polypeptide being at least 70%, optionally at least about 80%, preferably at least about 85%, more preferably at least about 90% and most preferably at least about 95% homologous to the sequence GEILSPPGP (SEQ ID NO:482) in HUM4COLA_PEA_{—}1_P15 (SEQ ID NO:278).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUM4COLA_PEA_{—}1_P15 (SEQ ID NO:278) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HUM4COLA_PEA_{—}1_P15 (SEQ ID NO:278) Sequence provides support for the deduced sequence of this variant protein according to the present invention).

TABLE 13
Amino acid mutations

| SNP position(s) on | Alternative amino | Previously |
|---|---|---|
| amino acid sequence | acid(s) | known SNP? |
| 6 | P -> | No |
| 20 | A -> | No |
| 20 | A -> V | Yes |
| 65 | K -> E | No |
| 82 | E -> G | No |
| 82 | E -> K | Yes |
| 113 | D -> | No |
| 127 | N -> K | Yes |
| 160 | Y -> * | Yes |
| 165 | D -> N | Yes |
| 170 | F -> | No |
| 174 | E -> | No |
| 190 | H -> R | No |
| 205 | D -> G | No |
| 218 | E -> * | Yes |

The glycosylation sites of variant protein HUM4COLA_PEA_{—}1_P15 (SEQ ID NO:278), as compared to the known protein 92 kDa type IV collagenase precursor (SEQ ID NO:275), are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known | Present in | Position in |
|---|---|---|
| amino acid sequence | variant protein? | variant protein? |
| 38 | yes | 38 |
| 127 | yes | 127 |
| 120 | yes | 120 |

Variant protein HUM4COLA_PEA_{—}1_P15 (SEQ ID NO:278) is encoded by the following transcript(s): HUM4COLA_PEA_{—}1_T5 (SEQ ID NO:246), for which the sequence(s) is/are given at the end of the application. The coding portion of transcript HUM4COLA_PEA_{—}1_T5 (SEQ ID NO:246) is shown in bold; this coding portion starts at position 33 and ends at position 707. The transcript also has the following SNPs as listed in Table 15 (given according to their position on the nucleotide sequence, with the alternative nucleic acid listed; the last column indicates whether the SNP is known or not; the presence of known SNPs in variant protein HUM4COLA_PEA_{—}1_P15 (SEQ ID NO:278) Sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 48 | C -> | No |
| 91 | C -> | No |
| 91 | C -> T | Yes |
| 225 | A -> G | No |
| 276 | G -> A | Yes |
| 277 | A -> G | No |
| 371 | C -> | No |
| 413 | C -> G | Yes |
| 503 | T -> C | No |
| 512 | C -> G | Yes |
| 525 | G -> A | Yes |
| 540 | T -> | No |
| 554 | G -> | No |
| 601 | A -> G | No |
| 646 | A -> G | No |
| 684 | G -> T | Yes |
| 790 | T -> G | No |
| 805 | -> A | No |
| 805 | -> T | No |
| 811 | C -> | No |
| 835 | G -> | No |
| 887 | T -> C | No |
| 960 | G -> A | Yes |
| 1010 | G -> | No |
| 1040 | G -> | No |
| 1062 | G -> | No |
| 1374 | C -> | No |
| 1464 | C -> T | Yes |
| 1470 | C -> T | Yes |
| 1761 | G -> | No |
| 1845 | C -> G | Yes |
| 1879 | C -> A | No |
| 1918 | G -> A | No |
| 1945 | A -> C | Yes |
| 1960 | G -> A | Yes |
| 1986 | G -> A | Yes |
| 2068 | C -> A | No |
| 2069 | G -> A | No |
| 2127 | G -> A | Yes |
| 2146 | C -> T | Yes |
| 2206 | G -> A | Yes |
| 2251 | C -> T | Yes |
| 2266 | G -> | No |
| 2287 | A -> C | Yes |
| 2304 | G -> A | Yes |
| 2344 | C -> T | Yes |
| 2394 | T -> C | Yes |
| 2438 | T -> | No |
| 2438 | T -> C | No |
| 2439 | T -> A | No |
| 2439 | T -> C | No |

As noted above, cluster HUM4COLA features 27 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUM4COLA_PEA_{—}1_node_{—}0 (SEQ ID NO:248) according to the present invention is supported by 53 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 16 below describes the starting and ending position of this segment on each transcript.

TABLE 16
Segment location on transcripts

| | Segment | Segment |
|---|---|---|
| Transcript name | starting position | ending position |
| HUM4COLA_PEA_1_T1 (SEQ ID | 1 | 170 |
| NO: 245) | | |
| HUM4COLA_PEA_1_T5 (SEQ ID | 1 | 170 |
| NO: 246) | | |
| HUM4COLA_PEA_1_T6 (SEQ ID | 1 | 170 |
| NO: 247) | | |

Segment cluster HUM4COLA_PEA_{—}1_node_{—}0 (SEQ ID NO:249) according to the present invention is supported by 60 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 17 below describes the starting and ending position of this segment on each transcript.

TABLE 17
Segment location on transcripts

| | Segment | Segment |
|---|---|---|
| Transcript name | starting position | ending position |
| HUM4COLA_PEA_1_T1 (SEQ ID | 171 | 403 |
| NO: 245) | | |
| HUM4COLA_PEA_1_T5 (SEQ ID | 171 | 403 |
| NO: 246) | | |
| HUM4COLA_PEA_1_T6 (SEQ ID | 171 | 403 |
| NO: 247) | | |

Segment cluster HUM4COLA_PEA_{—}1_node_{—}4 (SEQ ID NO:250) according to the present invention is supported by 51 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA-PEA_{—}1_T6 (SEQ ID NO:247). Table 18 below describes the starting and ending position of this segment on each transcript.

TABLE 18
Segment location on transcripts

| | Segment | Segment |
|---|---|---|
| Transcript name | starting position | ending position |
| HUM4COLA_PEA_1_T1 (SEQ ID | 404 | 552 |
| NO: 245) | | |
| HUM4COLA_PEA_1_T5 (SEQ ID | 404 | 552 |
| NO: 246) | | |
| HUM4COLA_PEA_1_T6 (SEQ ID | 404 | 552 |
| NO: 247) | | |

Segment cluster HUM4COLA_PEA_{—}1_node_{—}7 (SEQ ID NO:251) according to the present invention is supported by 64 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 19 below describes the starting and ending position of this segment on each transcript.

TABLE 19
Segment location on transcripts

| | Segment | Segment |
|---|---|---|
| Transcript name | starting position | ending position |
| HUM4COLA_PEA_1_T1 (SEQ ID | 553 | 681 |
| NO: 245) | | |
| HUM4COLA_PEA_1_T5 (SEQ ID | 553 | 681 |
| NO: 246) | | |
| HUM4COLA_PEA_1_T6 (SEQ ID | 553 | 681 |
| NO: 247) | | |

Segment cluster HUM4COLA_PEA_{—}1_node_{—}1 (SEQ ID NO:252) according to the present invention is supported by 2 libraries. The number of libraries was determined as previously described. This segment can be found in the following transcript(s): HUM4COLA_PEA_{—}1_T1 (SEQ ID NO:245). Table 20 below describes the starting and ending position of this segment on each transcript.

TABLE 20
Segment location on transcripts

| | Segment | Segment |
|---|---|---|
| Transcript name | starting position | ending position |
| HUM4COLA_PEA_1_T1 (SEQ ID | 856 | 1112 |
| NO: 245) | | |

Segment cluster HUM4COLA_PEA_{—}1_node_{—}19 (SEQ ID NO:253) according to the present invention is supported by 81 libraries. The number of libraries was determined as previously described. This segment can be found in the following transcript(s): HUM4COLA_PEA_{—}1_T1 (SEQ ID NO:245) and HUM4COLA_PEA_{—}1_T5 (SEQ ID NO:246). Table 21 below describes the starting and ending position of this segment on each transcript.

TABLE 21
Segment location on transcripts

| | Segment | Segment |
|---|---|---|
| Transcript name | starting position | ending position |
| HUM4COLA_PEA_1_T1 (SEQ ID | 1464 | 1619 |
| NO: 245) | | |
| HUM4COLA_PEA_1_T5 (SEQ ID | 1299 | 1454 |
| NO: 246) | | |

Segment cluster HUM4COLA_PEA_{—}1_node_{—}40 (SEQ ID NO:254) according to the present invention is supported by 129 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 22 below describes the starting and ending position of this segment on each transcript.

TABLE 22
Segment location on transcripts

| | Segment | Segment |
|---|---|---|
| Transcript name | starting position | ending position |
| HUM4COLA_PEA_1_T1 (SEQ ID | 2295 | 2453 |
| NO: 245) | | |
| HUM4COLA_PEA_1_T5 (SEQ ID | 2130 | 2288 |
| NO: 246) | | |
| HUM4COLA_PEA_1_T6 (SEQ ID | 1778 | 1936 |
| NO: 247) | | |

Segment cluster HUM4COLA_PEA_{—}1_node_{—}41 (SEQ ID NO:255) according to the present invention is supported by 112 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 23 below describes the starting and ending position of this segment on each transcript.

TABLE 23
Segment location on transcripts

| | Segment | Segment |
|---|---|---|
| Transcript name | starting position | ending position |
| HUM4COLA_PEA_1_T1 (SEQ ID | 2454 | 2616 |
| NO: 245) | | |
| HUM4COLA_PEA_1_T5 (SEQ ID | 2289 | 2451 |
| NO: 246) | | |
| HUM4COLA_PEA_1_T6 (SEQ ID | 1937 | 2099 |
| NO: 247) | | |

According to an optional embodiment of the present invention, short segments related to the above cluster are also provided. These segments are up to about 120 bp in length, and so are included in a separate description.

Segment cluster HUM4COLA_PEA_{—}1_node_{—}8 (SEQ ID NO:256) according to the present invention is supported by 1 libraries. The number of libraries was determined as previously described. This segment can be found in the following transcript(s): HUM4COLA_PEA_{—}1_T5 (SEQ ID NO:246). Table 24 below describes the starting and ending position of this segment on each transcript.

TABLE 24
Segment location on transcripts

| | Segment | Segment |
|---|---|---|
| Transcript name | starting position | ending position |
| HUM4COLA_PEA_1_T5 (SEQ ID | 682 | 773 |
| NO: 246) | | |

Segment cluster HUM4COLA_PEA_{—}1_node_{—}9 (SEQ ID NO:257) according to the present invention is supported by 59 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 25 below describes the starting and ending position of this segment on each transcript.

TABLE 25
Segment location on transcripts

| | Segment | Segment |
|---|---|---|
| Transcript name | starting position | ending position |
| HUM4COLA_PEA_1_T1 (SEQ ID | 682 | 736 |
| NO: 245) | | |
| HUM4COLA_PEA_1_T5 (SEQ ID | 774 | 828 |
| NO: 246) | | |
| HUM4COLA_PEA_1_T6 (SEQ ID | 682 | 736 |
| NO: 247) | | |

Segment cluster HUM4COLA_PEA_{—}1_node_{—}10 (SEQ ID NO:258) according to the present invention is supported by 63 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 26 below describes the starting and ending position of this segment on each transcript.

TABLE 26
Segment location on transcripts

| | Segment | Segment |
|---|---|---|
| Transcript name | starting position | ending position |
| HUM4COLA_PEA_1_T1 (SEQ ID | 737 | 855 |
| NO: 245) | | |
| HUM4COLA_PEA_1_T5 (SEQ ID | 829 | 947 |
| NO: 246) | | |
| HUM4COLA_PEA_1_T6 (SEQ ID | 737 | 855 |
| NO: 247) | | |

Segment cluster HUM4COLA_PEA_{—}1_node_{—}12 (SEQ ID NO:259) according to the present invention is supported by 60 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 27 below describes the starting and ending position of this segment on each transcript.

TABLE 27
Segment location on transcripts

| | Segment | Segment |
|---|---|---|
| Transcript name | starting position | ending position |
| HUM4COLA_PEA_1_T1 (SEQ ID | 1113 | 1167 |
| NO: 245) | | |
| HUM4COLA_PEA_1_T5 (SEQ ID | 948 | 1002 |
| NO: 246) | | |
| HUM4COLA_PEA_1_T6 (SEQ ID | 856 | 910 |
| NO: 247) | | |

Segment cluster HUM4COLA_PEA_{—}1_node_{—}13 (SEQ ID NO:260) according to the present invention is supported by 67 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 28 below describes the starting and ending position of this segment on each transcript.

TABLE 28
Segment location on transcripts

| | Segment | Segment |
|---|---|---|
| Transcript name | starting position | ending position |
| HUM4COLA_PEA_1_T1 (SEQ ID | 1168 | 1286 |
| NO: 245) | | |
| HUM4COLA_PEA_1_T5 (SEQ ID | 1003 | 1121 |
| NO: 246) | | |
| HUM4COLA_PEA_1_T6 (SEQ ID | 911 | 1029 |
| NO: 247) | | |

Segment cluster HUM4COLA_PEA_{—}1_node_{—}16 (SEQ ID NO:261) according to the present invention is supported by 73 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 29 below describes the starting and ending position of this segment on each transcript.

TABLE 29
Segment location on transcripts

| | Segment | Segment |
|---|---|---|
| Transcript name | starting position | ending position |
| HUM4COLA_PEA_1_T1 (SEQ ID | 1287 | 1359 |
| NO: 245) | | |
| HUM4COLA_PEA_1_T5 (SEQ ID | 1122 | 1194 |
| NO: 246) | | |
| HUM4COLA_PEA_1_T6 (SEQ ID | 1030 | 1102 |
| NO: 247) | | |

Segment cluster HUM4COLA_PEA_{—}1_node_{—}17 (SEQ ID NO:262) according to the present invention is supported by 79 libraries. The number of libraries was determined as previously described. This segment can be found in the following transcript(s): HUM4COLA_PEA_{—}1_T1 (SEQ ID NO:245) and HUM4COLA_PEA_{—}1_T5 (SEQ ID NO:246). Table 30 below describes the starting and ending position of this segment on each transcript.

TABLE 30
Segment location on transcripts

| | Segment | Segment |
|---|---|---|
| Transcript name | starting position | ending position |
| HUM4COLA_PEA_1_T1 (SEQ ID | 1360 | 1463 |
| NO: 245) | | |
| HUM4COLA_PEA_1_T5 (SEQ ID | 1195 | 1298 |
| NO: 246) | | |

Segment cluster HUM4COLA_PEA_{—}1_node_{—}22 (SEQ ID NO:263) according to the present invention is supported by 66 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 31 below describes the starting and ending position of this segment on each transcript.

TABLE 31
Segment location on transcripts

| | Segment | Segment |
|---|---|---|
| Transcript name | starting position | ending position |
| HUM4COLA_PEA_1_T1 (SEQ ID | 1620 | 1663 |
| NO: 245) | | |
| HUM4COLA_PEA_1_T5 (SEQ ID | 1455 | 1498 |
| NO: 246) | | |
| HUM4COLA_PEA_1_T6 (SEQ ID | 1103 | 1146 |
| NO: 247) | | |

Segment cluster HUM4COLA_PEA_{—}1_node-23 (SEQ ID NO:264) according to the present invention can be found in the following transcript(s): HUM4COLA_PEA_{—}1_T1 (SEQ ID NO:245), HUM4COLA_PEA_{—}1_T5 (SEQ ID NO:246) and HUM4COLA-PEA_{—}1_T6 (SEQ ID NO:247). Table 32 below describes the starting and ending position of this segment on each transcript.

TABLE 32
Segment location on transcripts

| | Segment | Segment |
|---|---|---|
| Transcript name | starting position | ending position |
| HUM4COLA_PEA_1_T1 (SEQ ID | 1664 | 1682 |
| NO: 245) | | |
| HUM4COLA_PEA_1_T5 (SEQ ID | 1499 | 1517 |
| NO: 246) | | |
| HUM4COLA_PEA_1_T6 (SEQ ID | 1147 | 1165 |
| NO: 247) | | |

Segment cluster HUM4COLA_PEA_{—}1_node_{—}24 (SEQ ID NO:265) according to the present invention is supported by 52 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 33 below describes the starting and ending position of this segment on each transcript.

TABLE 33
Segment location on transcripts

| | Segment | Segment |
|---|---|---|
| Transcript name | starting position | ending position |
| HUM4COLA_PEA_1_T1 (SEQ ID | 1683 | 1780 |
| NO: 245) | | |
| HUM4COLA_PEA_1_T5 (SEQ ID | 1518 | 1615 |
| NO: 246) | | |
| HUM4COLA_PEA_1_T6 (SEQ ID | 1166 | 1263 |
| NO: 247) | | |

Segment cluster HUM4COLA_PEA_{—}1_node_{—}25 (SEQ ID NO:266) according to the present invention is supported by 46 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 34 below describes the starting and ending position of this segment on each transcript.

TABLE 34
Segment location on transcripts

| | Segment | Segment |
|---|---|---|
| Transcript name | starting position | ending position |
| HUM4COLA_PEA_1_T1 (SEQ ID | 1781 | 1833 |
| NO: 245) | | |
| HUM4COLA_PEA_1_T5 (SEQ ID | 1616 | 1668 |
| NO: 246) | | |
| HUM4COLA_PEA_1_T6 (SEQ ID | 1264 | 1316 |
| NO: 247) | | |

Segment cluster HUM4COLA_PEA_{—}1_node_{—}26 (SEQ ID NO:267) according to the present invention is supported by 55 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 35 below describes the starting and ending position of this segment on each transcript.

TABLE 35
Segment location on transcripts

| | Segment | Segment |
|---|---|---|
| Transcript name | starting position | ending position |
| HUM4COLA_PEA_1_T1 (SEQ ID | 1834 | 1893 |
| NO: 245) | | |
| HUM4COLA_PEA_1_T5 (SEQ ID | 1669 | 1728 |
| NO: 246) | | |
| HUM4COLA_PEA_1_T6 (SEQ ID | 1317 | 1376 |
| NO: 247) | | |

Segment cluster HUM4COLA_PEA_{—}1_node_{—}27 (SEQ ID NO:268) according to the present invention can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 36 below describes the starting and ending position of this segment on each transcript.

TABLE 36
Segment location on transcripts

| | Segment | Segment |
|---|---|---|
| Transcript name | starting position | ending position |
| HUM4COLA_PEA_1_T1 (SEQ ID | 1894 | 1899 |
| NO: 245) | | |
| HUM4COLA_PEA_1_T5 (SEQ ID | 1729 | 1734 |
| NO: 246) | | |
| HUM4COLA_PEA_1_T6 (SEQ ID | 1377 | 1382 |
| NO: 247) | | |

Segment cluster HUM4COLA-PEA_{—}1_node_{—}29 (SEQ ID NO:269) according to the present invention is supported by 86 libraries. The number of libraries was determined as previously described. This segment can be found in the following transcript(s): HUM4COLA-PEA_{—}1_T1 (SEQ ID NO:245), HUM4COLA PEA_{—}1_T5 (SEQ ID NO:246) and HUM4COLA_PEA_{—}1_T6 (SEQ ID NO:247). Table 37 below describes the starting and ending position of this segment on each transcript.

TABLE 37
Segment location on transcripts

| | Segment | Segment |
|---|---|---|
| Transcript name | starting position | ending position |
| HUM4COLA_PEA_1_T1 (SEQ ID | 1900 | 2008 |
| NO: 245) | | |
| HUM4COLA_PEA_1_T5 (SEQ ID | 1735 | 1843 |
| NO: 246) | | |
| HUM4COLA_PEA_1_T6 (SEQ ID | 1383 | 1491 |
| NO: 247) | | |

Segment cluster HUM4COLA_PEA_{—}1_node_{—}30 (SEQ ID NO:270) according to the present invention is supported by 83 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 38 below describes the starting and ending position of this segment on each transcript.

TABLE 38
Segment location on transcripts

| | Segment | Segment |
|---|---|---|
| Transcript name | starting position | ending position |
| HUM4COLA_PEA_1_T1 (SEQ ID | 2009 | 2039 |
| NO: 245) | | |
| HUM4COLA_PEA_1_T5 (SEQ ID | 1844 | 1874 |
| NO: 246) | | |
| HUM4COLA_PEA_1_T6 (SEQ ID | 1492 | 1522 |
| NO: 247) | | |

Segment cluster HUM4COLA_PEA_{—}1_node_{—}32 (SEQ ID NO:271) according to the present invention is supported by 103 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 39 below describes the starting and ending position of this segment on each transcript.

TABLE 39
Segment location on transcripts

| | Segment | Segment |
|---|---|---|
| Transcript name | starting position | ending position |
| HUM4COLA_PEA_1_T1 (SEQ ID | 2040 | 2158 |
| NO: 245) | | |
| HUM4COLA_PEA_1_T5 (SEQ ID | 1875 | 1993 |
| NO: 246) | | |
| HUM4COLA_PEA_1_T6 (SEQ ID | 1523 | 1641 |
| NO: 247) | | |

Segment cluster HUM4COLA_PEA_{—}1_node_{—}33 (SEQ ID NO:272) according to the present invention is supported by 101 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 40 below describes the starting and ending position of this segment on each transcript.

TABLE 40
Segment location on transcripts

| | Segment | Segment |
|---|---|---|
| Transcript name | starting position | ending position |
| HUM4COLA_PEA_1_T1 (SEQ ID | 2159 | 2190 |
| NO: 245) | | |
| HUM4COLA_PEA_1_T5 (SEQ ID | 1994 | 2025 |
| NO: 246) | | |
| HUM4COLA_PEA_1_T6 (SEQ ID | 1642 | 1673 |
| NO: 247) | | |

Segment cluster HUM4COLA_PEA_{—}1_node_{—}36 (SEQ ID NO:273) according to the present invention is supported by 108 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 41 below describes the starting and ending position of this segment on each transcript.

TABLE 41
Segment location on transcripts

| | Segment | Segment |
|---|---|---|
| Transcript name | starting position | ending position |
| HUM4COLA_PEA_1_T1 (SEQ ID | 2191 | 2242 |
| NO: 245) | | |
| HUM4COLA_PEA_1_T5 (SEQ ID | 2026 | 2077 |
| NO: 246) | | |
| HUM4COLA_PEA_1_T6 (SEQ ID | 1674 | 1725 |
| NO: 247) | | |

Segment cluster HUM4COLA_PEA_{—}1_node_{—}37 (SEQ ID NO:274) according to the present invention is supported by 118 libraries. The number of libraries was determined as previously described. This segment can be found in the following transcript(s): HUM4COLA_PEA_{—}1_T1 (SEQ ID NO:245), HUM4COLA_PEA_{—}1_T5 (SEQ ID NO:246) and HUM4COLA_PEA_{—}1_T6 (SEQ ID NO:247). Table 42 below describes the starting and ending position of this segment on each transcript.

TABLE 42
Segment location on transcripts

| | Segment | Segment |
|---|---|---|
| Transcript name | starting position | ending position |
| HUM4COLA_PEA_1_T1 (SEQ ID | 2243 | 2294 |
| NO: 245) | | |
| HUM4COLA_PEA_1_T5 (SEQ ID | 2078 | 2129 |
| NO: 246) | | |
| HUM4COLA_PEA_1_T6 (SEQ ID | 1726 | 1777 |
| NO: 247) | | |

Variant protein alignment to the previously known protein:

| Sequence name: MM09_HUMAN (SEQ ID NO:275) | | | |
|---|---|---|---|
| Sequence documentation: | | | |
| Alignment of: HUM4COLA_PEA_1_P7 (SEQ ID NO:276) × | | | |
| MM09_HUMAN (SEQ ID NO:275) . . | | | |
| Alignment segment 1/1: | | | |
| Quality: | 3559.00 | Escore: | 0 |
| Matching length: | 359 | Total length: | 359 |
| Matching Percent | 99.72 | Matching Percent | 99.72 |
| Similarity: | | Identity: | |
| Total Percent | 99.72 | Total Percent | 99.72 |
| Similarity: | | Identity: | |
| Gaps: | 0 | | |

| Alignment: | | |
|---|---|---|
| | . . . . . | |
| 1 | MSLWQPLVLVLLVLGCCFAAPRQRQSTLVLFPGDLRTNLTDRQLAEEYLY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSLWQPLVLVLLVLGCCFAAPRQRQSTLVLFPGDLRTNLTDRQLAEEYLY | 50 |
| | . . . . . | |
| 51 | RYGYTRVAEMRGESKSLGPALLLLQKQLSLPETGELDSATLKAMRTPRCG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | RYGYTRVAEMRGESKSLGPALLLLQKQLSLPETGELDSATLKANRTPRCG | 100 |
| | . . . . . | |
| 101 | VPDLGRFQTFEGDLKWHHHNITYWIQNYSEDLPRAVIDDAFARAFALWSA | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | VPDLGRFQTFEGDLKWHHHNITYWIQNYSEDLPRAVIDDAFARAFALWSA | 150 |
| | . . . . . | |
| 151 | VTPLTFTRVYSRDADIVIQFGVAEHGDGYPFDGKDGLLAHAFPPGPGIQG | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | VTPLTFTRVYSRDADIVIQFGVAEHGDGYPFDGKDGLLAHAFPPGPGIQG | 200 |
| | . . . . . | |
| 201 | DAHFDDDELWSLGKGVVVPTRFGNADGAACHFPFIFEGRSYSACTTDGRS | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DAHFDDDELWSLGKGVVVPTRFGNADGAACHFPFIFEGRSYSACTTDGRS | 250 |
| | . . . . . | |
| 251 | DGLPWCSTTANYDTDDRFGFCPSERLYTRDGNADGKPCQFPFIFQGQSYS | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | DGLPWCSTTANYDTDDRFGFCPSERLYTRDGNADGKPCQFPFIFQGQSYS | 300 |
| | . . . . . | |
| 301 | ACTTDGRSDGYRWCATTANYDRDKLFGFCPTRADSTVMGGNSAGELCVFP | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | ACTTDGRSDGYRWCATTANYDRDKLFGFCPTRADSTVMGGNSAGELCVFP | 350 |
| 351 | FTFLGKESS | 359 |
| | \|\|\|\|\|\|\|\|\| | |
| 351 | FTFLGKEYS | 359 |

| Sequence name: MM09_HUMAN (SEQ ID NO:275) | | | |
|---|---|---|---|
| Sequence documentation: | | | |
| Alignment of: HUM4COLA_PEA_1_P14 (SEQ ID NO:277) × | | | |
| MM09_HUMAN (SEQ ID NO:275) . . | | | |
| Alignment segment 1/1: | | | |
| Quality: | 2715.00 | Escore: | 0 |
| Matching length: | 274 | Total length: | 274 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MSLWQPLVLVLLVLGCCFAAPRQRQSTLVLFPGDLRTNLTDRQLAEEYLY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSLWQPLVLVLLVLGCCFAAPRQRQSTLVLFPGDLRTNLTDRQLAEEYLY | 50 |
| | . . . . . | |
| 51 | RYGYTRVAEMRGESKSLGPALLLLQKQLSLPETGELDSATLKANRTPRCG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | RYGYTRVAEMRGESKSLGPALLLLQKQLSLPETGELDSATLKANRTPRCG | 100 |
| | . . . . . | |
| 101 | VPDLGRFQTFEGDLKWHHHNITYWIQNYSEDLPRAVIDDAFARAFALWSA | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | VPDLGRFQTFEGDLKWHHHNITYWIQNYSEDLPRAVIDDAFARAFALWSA | 150 |
| | . . . . . | |
| 151 | VTPLTFTRVYSRDADIVIQFGVAEHGDGYPFDGKDGLLAHAFPPGPGIQG | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | VTPLTFTRVYSRDADIVIQFGVAEHGDGYPFDGKDGLLAHAFPPGPGIQG | 200 |
| | . . . . . | |
| 201 | DAHFDDDELWSLGKGVVVPTRFGNADGAACHFPFIFEGRSYSACTTDGRS | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DAHFDDDELWSLGKGVVVPTRFGNADGAACHFPFIFEGRSYSACTTDGRS | 250 |
| | . . | |
| 251 | DGLPWCSTTANYDTDDRFGFCPSE | 274 |
| | \|\|\|\|\|\|\|\|\|\|\|\|\|\|\|\|\|\|\|\|\|\|\|\| | |
| 251 | DGLPWCSTTANYDTDDRFGFCPSE | 274 |

| Sequence name: MM09_HUMAN (SEQ ID NO:275) | | | |
|---|---|---|---|
| Sequence documentation: | | | |
| Alignment of: HUM4COLA_PEA_1_P15 (SEQ ID NO:278) × | | | |
| MM09_HUMAN (SEQ ID NO:275) . . | | | |
| Alignment segment 1/1: | | | |
| Quality: | 2124.00 | Escore: | 0 |
| Matching length: | 216 | Total length: | 216 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MSLWQPLVLVLLVLGCCFAAPRQRQSTLVLFPGDLRTNLTDRQLAEEYLY | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSLWQPLVLVLLVLGCCFAAPRQRQSTLVLFPGDLRTNLTDRQLAEEYLY | 50 |
| | . . . . . | |
| 51 | RYGYTRVAEMRGESKSLGPALLLLQKQLSLPETGELDSATLKAMRTPRCG | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | RYGYTRVAEMRGESKSLGPALLLLQKQLSLPETGELDSATLKAMRTPRCG | 100 |
| | . . . . . | |
| 101 | VPDLGRFQTFEGDLKWHHHNITYWIQNYSEDLPRAVIDDAFARAFALWSA | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | VPDLGRFQTFEGDLKWHHHNITYWIQNYSEDLPRAVIDDAFARAFALWSA | 150 |
| | . . . . . | |
| 151 | VTPLTFTRVYSRDADIVIQFGVAEHGDGYPFDGKDGLLAHAFPPGPGIQG | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | VTPLTFTRVYSRDADIVIQFGVAEHGDGYPFDGKDGLLAHAFPPGPGIQG | 200 |
| | . | |
| 201 | DAHFDDDELWSLGKGV | 216 |
| | \|\|\|\|\|\|\|\|\|\|\|\|\|\|\|\| | |
| 201 | DAHFDDDELWSLGKGV | 216 |

### Description for Cluster HUMICAMA1A

Cluster HUMICAMA1A features 6 transcript(s) and 22 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HUMICAMA1A_PEA_1_T2 | 279 |
| HUMICAMA1A_PEA_1_T4 | 280 |
| HUMICAMA1A_PEA_1_T5 | 281 |
| HUMICAMA1A_PEA_1_T8 | 282 |
| HUMICAMA1A_PEA_1_T12 | 283 |
| HUMICAMA1A_PEA_1_T16 | 284 |

TABLE 2
Segments of interest

| Segment Name | Sequence ID No. |
|---|---|
| HUMICAMA1A_PEA_1_node_0 | 285 |
| HUMICAMA1A_PEA_1_node_3 | 286 |
| HUMICAMA1A_PEA_1_node_12 | 287 |
| HUMICAMA1A_PEA_1_node_13 | 288 |
| HUMICAMA1A_PEA_1_node_14 | 289 |
| HUMICAMA1A_PEA_1_node_20 | 290 |
| HUMICAMA1A_PEA_1_node_21 | 291 |
| HUMICAMA1A_PEA_1_node_24 | 292 |
| HUMICAMA1A_PEA_1_node_25 | 293 |
| HUMICAMA1A_PEA_1_node_27 | 294 |
| HUMICAMA1A_PEA_1_node_29 | 295 |
| HUMICAMA1A_PEA_1_node_2 | 296 |
| HUMICAMA1A_PEA_1_node_4 | 297 |
| HUMICAMA1A_PEA_1_node_15 | 298 |
| HUMICAMA1A_PEA_1_node_16 | 299 |
| HUMICAMA1A_PEA_1_node_17 | 300 |
| HUMICAMA1A_PEA_1_node_18 | 301 |
| HUMICAMA1A_PEA_1_node_19 | 302 |
| HUMICAMA1A_PEA_1_node_22 | 303 |
| HUMICAMA1A_PEA_1_node_23 | 304 |
| HUMICAMA1A_PEA_1_node_26 | 305 |
| HUMICAMA1A_PEA_1_node_28 | 306 |

TABLE 3
Proteins of interest

| Protein Name | Sequence ID No. | Corresponding Transcript(s) |
|---|---|---|
| HUMICAMA1A_PEA_1_P2 | 309 | HUMICAMA1A_PEA_1_T2 (SEQ |
| | | ID NO: 279) |
| HUMICAMA1A_PEA_1_P5 | 310 | HUMICAMA1A_PEA_1_T5 |
| | | (SEQ ID NO: 281); |
| | | HUMICAMA1A_PEA_1_T12 |
| | | (SEQ ID NO: 283); |
| | | HUMICAMA1A_PEA_1_T16 |
| | | (SEQ ID NO: 284) |
| HUMICAMA1A_PEA_1_P8 | 311 | HUMICAMA1A_PEA_1_T8 |
| | | (SEQ ID NO: 282) |
| HUMICAMA1A_PEA_1_P15 | 312 | HUMICAMA1A_PEA_1_T4 |
| | | (SEQ ID NO: 280) |

These sequences are variants of the known protein Intercellular adhesion molecule-1 precursor (SEQ ID NO:307) (SwissProt accession identifier ICA1_HUMAN; known also according to the synonyms ICAM-1; Major group rhinovirus receptor; CD54 antigen), referred to herein as the previously known protein.

Protein Intercellular adhesion molecule-1 precursor (SEQ ID NO:307) is known or believed to have the following function(s): ICAM proteins are ligands for the leukocyte adhesion LFA-1 protein (Integrin alpha-L/beta-2). The sequence for protein Intercellular adhesion molecule-1 precursor is given at the end of the application, as “Intercellular adhesion molecule-1 precursor amino acid sequence” (SEQ ID NO:307). Known polymorphisms for this sequence are as shown in Table 4.

TABLE 4
Amino acid mutations or Known Protein

| SNP position(s) | |
|---|---|
| on amino acid | |
| sequence | Comment |
| 56 | K -> M (in Kilifi; dbSNP: 5491). /FTId = VAR_010204. |
| 155 | K -> N (in dbSNP: 5492). /FTId = VAR_014651. |
| 241 | G -> R (in dbSNP: 1799969). /FTId = VAR_014186. |
| 315 | V -> M (in dbSNP: 5495). /FTId = VAR_014652. |
| 352 | P -> L (in dbSNP: 1801714). /FTId = VAR_014653. |
| 397 | R -> Q (in dbSNP: 5497). /FTId = VAR_014654. |
| 469 | E -> K (in dbSNP: 5498). /FTId = VAR_014187. |
| 478 | R -> W. /FTId = VAR_016267. |
| 9-10 | AL -> PV |

Protein Intercellular adhesion molecule-1 precursor (SEQ ID NO:307) localization is believed to be Type I membrane protein.

A lower serum concentration of soluble ICAM-1 is seen in women with stage III and IV endometriosis (Barrier et al, J Soc Gynecol Investig. 2002 March-April; 9(2):98-101). Variants of this cluster are suitable as diagnostic markers for endometriosis.

The previously known protein also has the following indication(s) and/or potential therapeutic use(s): Infection, rhinovirus. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: ICAM 1 antagonist; Immunostimulant; Protein synthesis antagonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anti-inflammatory; Immunological; antibody; Antiallergic, non-asthma; Otological; Antiviral; GI inflammatory/bowel disorders; Cardiovascular; Antipruritic/inflammation, allergic; Anti-inflammatory, topical; Antiarthritic, immunological; Antisense therapy; Anti-infective; Anticancer; Prophylactic vaccine.

The following GO Annotation(s) apply to the previously known protein. The following annotation(s) were found: cell-cell adhesion, which are annotation(s) related to Biological Process; transmembrane receptor; protein binding, which are annotation(s) related to Molecular Function; and integral plasma membrane protein, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster HUMICAMA1A features 6 transcript(s), which were listed in Table 1 above. These transcript(s) encode for protein(s) which are variant(s) of protein Intercellular adhesion molecule-1 precursor (SEQ ID NO:307). A description of each variant protein according to the present invention is now provided.

Variant protein HUMICAMA1A_PEA_{—}1_P2 (SEQ ID NO:309) according to the present invention has an amino acid sequence as given at the end of the application; it is encoded by transcript(s) HUMICAMA1A_PEA_{—}1_T2 (SEQ ID NO:279). An alignment is given to the known protein (Intercellular adhesion molecule-1 precursor (SEQ ID NO:307)) at the end of the application. One or more alignments to one or more previously published protein sequences are given at the end of the application. A brief description of the relationship of the variant protein according to the present invention to each such aligned protein is as follows:

Comparison report between HUMICAMA1A_PEA_{—}1_P2 (SEQ ID NO:309) and ICA1_HUMAN (SEQ ID NO:307):

1. An isolated chimeric polypeptide encoding for HUMICAMA1A_PEA_{—}1_P2 (SEQ ID NO:309), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTFVLPATPPQLVSPRVLEVDTQGTVV CSLDGLFPVSEAQVHLALGDQRLNPTVTYGNDSFSAKASVSVTAEDEGTQRLTCAVILG NQSQETLQTVTIYS corresponding to amino acids 1-309 of ICA1_HUMAN (SEQ ID NO:307), which also corresponds to amino acids 1-309 of HUMICAMA1A_PEA_{—}1_P2 (SEQ ID NO:309), and a second amino acid sequence being at least 70%, optionally at least 80%, preferably at least 85%, more preferably at least 90% and most preferably at least 95% homologous to a polypeptide having the sequence KKGQGRSGASWGCDLNPGRGSLCAYSRLSGAQRDSDEARGLRRDRGDSEV (SEQ ID NO:479) corresponding to amino acids 310-359 of HUMICAMA1A_PEA_{—}1_P2 (SEQ ID NO:309), wherein said first amino acid sequence and second amino acid sequence are contiguous and in a sequential order.

2. An isolated polypeptide encoding for a tail of HUMICAMA1A_PEA_{—}1_P2 (SEQ ID NO:309), comprising a polypeptide being at least 70%, optionally at least about 80%, preferably at least about 85%, more preferably at least about 90% and most preferably at least about 95% homologous to the sequence KKGQGRSGASWGCDLNPGRGSLCAYSRLSGAQRDSDEARGLRRDRGDSEV (SEQ ID NO:479) in HUMICAMA1A_PEA_{—}1_P2 (SEQ ID NO:309).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMICAMA1A_PEA_{—}1_P2 (SEQ ID NO:309) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUMICAMA1A_PEA_{—}1_P2 (SEQ ID NO:309) Sequence provides support for the deduced sequence of this variant protein according to the present invention).

TABLE 7
Amino acid mutations

| SNP position(s) on amino acid | | Previously |
|---|---|---|
| sequence | Alternative amino acid(s) | known SNP? |
| 56 | K -> M | Yes |
| 155 | K -> N | Yes |
| 238 | S -> | No |
| 241 | G -> R | Yes |
| 272 | A -> G | No |
| 272 | A -> | No |
| 285 | T -> A | No |
| 320 | W -> * | Yes |
| 342 | R -> H | Yes |

The glycosylation sites of variant protein HUMICAMA1A_PEA_{—}1_P2 (SEQ ID NO:309), as compared to the known protein Intercellular adhesion molecule-1 precursor (SEQ ID NO:307, are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on known amino | | Position in |
|---|---|---|
| acid sequence | Present in variant protein? | variant protein? |
| 385 | no | |
| 296 | yes | 296 |
| 202 | yes | 202 |
| 145 | yes | 145 |
| 130 | yes | 130 |
| 406 | no | |
| 183 | yes | 183 |
| 267 | yes | 267 |

Variant protein HUMICAMA1A_PEA_{—}1_P2 (SEQ ID NO:309) is encoded by the following transcript(s): HUMICAMA1A_PEA_{—}1_T2 (SEQ ID NO:279), for which the sequence(s) is/are given at the end of the application. The coding portion of transcript HUMICAMA1A_PEA_{—}1_T2 (SEQ ID NO:279) is shown in bold; this coding portion starts at position 1332 and ends at position 2408. The transcript also has the following SNPs as listed in Table 9 (given according to their position on the nucleotide sequence, with the alternative nucleic acid listed; the last column indicates whether the SNP is known or not; the presence of known SNPs in variant protein HUMICAMA1A_PEA_{—}1_P2 (SEQ ID NO:309) Sequence provides support for the deduced sequence of this variant protein according to the present invention).

TABLE 9
Nucleic acid SNPs

| SNP position on nucleotide | | Previously |
|---|---|---|
| sequence | Alternative nucleic acid | known SNP? |
| 1 | G -> C | No |
| 169 | G -> A | Yes |
| 490 | A -> | Yes |
| 1288 | C -> T | Yes |
| 1291 | T -> G | No |
| 1323 | A -> C | Yes |
| 1498 | A -> T | Yes |
| 1796 | G -> C | Yes |
| 2033 | C -> A | Yes |
| 2045 | C -> | No |
| 2052 | G -> A | Yes |
| 2054 | G -> T | Yes |
| 2146 | C -> | No |
| 2146 | C -> G | No |
| 2168 | C -> T | Yes |
| 2177 | C -> T | Yes |
| 2184 | A -> G | No |
| 2198 | G -> A | No |
| 2291 | G -> A | Yes |
| 2356 | G -> A | Yes |
| 2414 | C -> | No |
| 2414 | C -> G | No |
| 2468 | C -> T | Yes |
| 2508 | C -> T | Yes |
| 2534 | C -> | No |
| 2534 | C -> A | No |
| 2544 | G -> | No |
| 2598 | C -> | No |
| 2818 | A -> G | No |
| 2975 | C -> | No |
| 3064 | G -> T | No |
| 3119 | C -> | No |
| 3137 | G -> | No |
| 3446 | C -> | No |
| 3732 | T -> | No |
| 3732 | T -> C | No |
| 3859 | T -> A | No |
| 3866 | C -> T | No |
| 3982 | -> T | No |
| 4082 | -> G | No |
| 4082 | -> T | No |
| 4180 | G -> A | No |
| 4312 | T -> G | No |

Variant protein HUMICAMA1A_PEA_{—}1_P5 (SEQ ID NO:310) according to the present invention has an amino acid sequence as given at the end of the application; it is encoded by transcript(s) HUMICAMA1A_PEA_T5 (SEQ ID NO:281). An alignment is given to the known protein (Intercellular adhesion molecule-1 precursor (SEQ ID NO:307)) at the end of the application. One or more alignments to one or more previously published protein sequences are given at the end of the application. A brief description of the relationship of the variant protein according to the present invention to each such aligned protein is as follows:

Comparison report between HUMICAMA1A_PEA_{—}1_P5 (SEQ ID NO:310) and ICA1_HUMAN (SEQ ID NO:307):

1. An isolated chimeric polypeptide encoding for HUMICAMA1A_PEA_{—}1_P5 (SEQ ID NO:310), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTFVLPATPPQLVSPRVLEVDTQGTVV CSLDGLFPVSEAQVHLALGDQRLNPTVTYGNDSFSAKASVSVTAEDEGTQRLTCAVILG NQSQETLQTVTIYSFPAPNVILTKPEVSEGTEVTVKCEAHPRAKVTLNGVPAQPLGPRA QLLLKATPEDNGRSFSCSATLEVAGQLIHKNQTRELRVL corresponding to amino acids 1-393 of ICA1_HUMAN (SEQ ID NO:307), which also corresponds to amino acids 1-393 of HUMICAMA1A_PEA_{—}1_P5 (SEQ ID NO:310), and a second amino acid sequence being at least 70%, optionally at least 80%, preferably at least 85%, more preferably at least 90% and most preferably at least 95% homologous to a polypeptide having the sequence CEWGCWSMAPIPQGPISLKVP (SEQ ID NO:480) corresponding to amino acids 394-414 of HUMICAMA1A_PEA_{—}1_P5 (SEQ ID NO:310), wherein said first amino acid sequence and second amino acid sequence are contiguous and in a sequential order.

2. An isolated polypeptide encoding for a tail of HUMICAMA1A_PEA_{—}1_P5 (SEQ ID NO:310), comprising a polypeptide being at least 70%, optionally at least about 80%, preferably at least about 85%, more preferably at least about 90% and most preferably at least about 95% homologous to the sequence CEWGCWSMAPIPQGPISLKVP (SEQ ID NO:480) in HUMICAMA1A_PEA_{—}1_P5 (SEQ ID NO:310).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMICAMA1A_PEA_{—}1_P5 (SEQ ID NO:310) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HUMICAMA1A_PEA_{—}1_P5 (SEQ ID NO:310) Sequence provides support for the deduced sequence of this variant protein according to the present invention).

TABLE 10
Amino acid mutations

| SNP position(s) on amino acid | | Previously |
|---|---|---|
| sequence | Alternative amino acid(s) | known SNP? |
| 56 | K -> M | Yes |
| 155 | K -> N | Yes |
| 238 | S -> | No |
| 241 | G -> R | Yes |
| 272 | A -> | No |
| 272 | A -> G | No |
| 285 | T -> A | No |
| 315 | V -> M | Yes |
| 334 | A -> G | No |
| 334 | A -> | No |
| 352 | P -> L | Yes |
| 374 | T -> | No |
| 374 | T -> N | No |
| 377 | V -> | No |

The glycosylation sites of variant protein HUMICAMA1A_PEA_{—}1_P5 (SEQ ID NO:310), as compared to the known protein Intercellular adhesion molecule-1 precursor (SEQ ID NO:307), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | | Position in |
|---|---|---|
| acid sequence | Present in variant protein? | variant protein? |
| 385 | yes | 385 |
| 296 | yes | 296 |
| 202 | yes | 202 |
| 145 | yes | 145 |
| 130 | yes | 130 |
| 406 | no | |
| 183 | yes | 183 |
| 267 | yes | 267 |

Variant protein HUMICAMA1A_PEA_{—}1_P5 (SEQ ID NO:310) is encoded by the following transcript(s): HUMICAMA1A_PEA_{—}1_T5 (SEQ ID NO:281), for which the sequence(s) is/are given at the end of the application. The coding portion of transcript HUMICAMA1A_PEA_{—}1_T5 (SEQ ID NO:281) is shown in bold; this coding portion starts at position 1332 and ends at position 2573. The transcript also has the following SNPs as listed in Table 12 (given according to their position on the nucleotide sequence, with the alternative nucleic acid listed; the last column indicates whether the SNP is known or not; the presence of known SNPs in variant protein HUMICAMA1A_PEA_{—}1_P5 (SEQ ID NO:310) Sequence provides support for the deduced sequence of this variant protein according to the present invention).

TABLE 12
Nucleic acid SNPs

| SNP position on nucleotide | | Previously |
|---|---|---|
| sequence | Alternative nucleic acid | known SNP? |
| 1 | G -> C | No |
| 169 | G -> A | Yes |
| 490 | A -> | Yes |
| 1288 | C -> T | Yes |
| 1291 | T -> G | No |
| 1323 | A -> C | Yes |
| 1498 | A -> T | Yes |
| 1796 | G -> C | Yes |
| 2033 | C -> A | Yes |
| 2045 | C -> | No |
| 2052 | G -> A | Yes |
| 2054 | G -> T | Yes |
| 2146 | C -> | No |
| 2146 | C -> G | No |
| 2168 | C -> T | Yes |
| 2177 | C -> T | Yes |
| 2184 | A -> G | No |
| 2198 | G -> A | No |
| 2274 | G -> A | Yes |
| 2332 | C -> | No |
| 2332 | C -> G | No |
| 2386 | C -> T | Yes |
| 2426 | C -> T | Yes |
| 2452 | C -> | No |
| 2452 | C -> A | No |
| 2462 | G -> | No |
| 2641 | C -> | No |
| 2861 | A -> G | No |
| 3018 | C -> | No |
| 3107 | G -> T | No |
| 3162 | C -> | No |
| 3180 | G -> | No |
| 3489 | C -> | No |
| 3775 | T -> | No |
| 3775 | T -> C | No |
| 3902 | T -> A | No |
| 3909 | C -> T | No |
| 4025 | -> T | No |
| 4125 | -> G | No |
| 4125 | -> T | No |
| 4223 | G -> A | No |
| 4355 | T -> G | No |

Variant protein HUMICAMA1A_PEA_{—}1_P8 (SEQ ID NO:311) according to the present invention has an amino acid sequence as given at the end of the application; it is encoded by transcript(s) HUMICAMA1A_PEA_{—}1_T8 (SEQ ID NO:282). An alignment is given to the known protein (Intercellular adhesion molecule-1 precursor (SEQ ID NO:307)) at the end of the application. One or more alignments to one or more previously published protein sequences are given at the end of the application. A brief description of the relationship of the variant protein according to the present invention to each such aligned protein is as follows:

Comparison report between HUMICAMA1A_PEA_{—}1_P8 (SEQ ID NO:311) and ICA1_HUMAN-V1 (SEQ ID NO:308):

1. An isolated chimeric polypeptide encoding for HUMICAMA1A_PEA_{—}1_P8 (SEQ ID NO:311), comprising a first amino acid sequence being at least 90% homologous to MAPSSPRPALPALLVLLGALFPG corresponding to amino acids 1-23 of ICA1_HUMAN_V1 (SEQ ID NO:308), which also corresponds to amino acids 1-23 of HUMICAMA1A_PEA_{—}1_P8 (SEQ ID NO:311), and a second amino acid sequence being at least 90% homologous to TPERVELAPLPSWQPVGKNLTLRCQVEGGAPRANLTVVLLRGEKELKREPAVGEPAEV TTTVLVRRDHHGANFSCRTELDLRPQGLELFENTSAPYQLQTFVLPATPPQLVSPRVLE VDTQGTVVCSLDGLFPVSEAQVHLALGDQRLNPTVTYGNDSFSAKASVSVTAEDEGTQ RLTCAVILGNQSQETLQTVTIYSFPAPNVILTKPEVSEGTEVTVKCEAHPRAKVTLNGVP AQPLGPRAQLLLKATPEDNGRSFSCSATLEVAGQLIHKNQTRELRVLYGPRLDERDCPG NWTWPENSQQTPMCQAWGNPLPELKCLKDGTFPLPIGESVTVTRDLEGTYLCRARSTQ GEVTRKVTVNVLSPRYEIVIITVVAAAVIMGTAGLSTYLYNRQRKIKKYRLQQAQKGTP MKPNTQATPP corresponding to amino acids 112-532 of ICA1_HUMAN_V1 (SEQ ID NO:308), which also corresponds to amino acids 24-444 of HUMICAMA1A_PEA_{—}1_P8 (SEQ ID NO:311), wherein said first amino acid sequence and second amino acid sequence are contiguous and in a sequential order.

2. An isolated chimeric polypeptide encoding for an edge portion of HUMICAMA1A_PEA_{—}1_P8 (SEQ ID NO:311), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GT, having a structure as follows: a sequence starting from any of amino acid numbers 23−x to 23; and ending at any of amino acid numbers 24+((n−2)−x), in which x varies from 0 to n−2.

It should be noted that the known protein sequence (ICA1_HUMAN (SEQ ID NO:307)) has one or more changes than the sequence given at the end of the application and named as being the amino acid sequence for ICA1_HUMAN_V1 (SEQ ID NO:308). These changes were previously known to occur and are listed in the table below.

TABLE 13
Changes to ICA1_HUMAN_V1 (SEQ ID NO: 308)

| SNP position(s) on | |
|---|---|
| amino acid sequence | Type of change |
| 470 | variant |

The location of the variant protein was determined according to results from a number of different software programs and analyses, including analyses from SignalP and other specialized programs. The variant protein is believed to be located as follows with regard to the cell: membrane. The protein localization is believed to be membrane because although both signal-peptide prediction programs agree that this protein has a signal peptide, both trans-membrane region prediction programs predict that this protein has a trans-membrane region downstream of this signal peptide.

Variant protein HUMICAMA1A_PEA_{—}1_P8 (SEQ ID NO:311) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HUMICAMA1A_PEA_{—}1_P8 (SEQ ID NO:311) Sequence provides support for the deduced sequence of this variant protein according to the present invention).

TABLE 14
Amino acid mutations

| SNP position(s) on | | |
|---|---|---|
| amino acid sequence | Alternative amino acid(s) | Previously known SNP? |
| 67 | K -> N | Yes |
| 150 | S -> | No |
| 153 | G -> R | Yes |
| 184 | A -> | No |
| 184 | A -> G | No |
| 197 | T -> A | No |
| 227 | V -> M | Yes |
| 246 | A -> G | No |
| 246 | A -> | No |
| 264 | P -> L | Yes |
| 286 | T -> | No |
| 286 | T -> N | No |
| 289 | V -> | No |
| 307 | G -> | No |
| 381 | K -> E | No |
| 433 | T -> | No |

Variant protein HUMICAMA1A_PEA_{—}1_P8 (SEQ ID NO:311) is encoded by the following transcript(s): HUMICAMA1A_PEA_{—}1_T8 (SEQ ID NO:282), for which the sequence(s) is/are given at the end of the application. The coding portion of transcript HUMICAMA1A_PEA_{—}1_T8 (SEQ ID NO:282) is shown in bold; this coding portion starts at position 1332 and ends at position 2663. The transcript also has the following SNPs as listed in Table 15 (given according to their position on the nucleotide sequence, with the alternative nucleic acid listed; the last column indicates whether the SNP is known or not; the presence of known SNPs in variant protein HUMICAMA1A_PEA_{—}1_P8 (SEQ ID NO:311) Sequence provides support for the deduced sequence of this variant protein according to the present invention).

TABLE 15
Nucleic acid SNPs

| SNP position on | | |
|---|---|---|
| nucleotide sequence | Alternative nucleic acid | Previously known SNP? |
| 1 | G -> C | No |
| 169 | G -> A | Yes |
| 490 | A -> | Yes |
| 1288 | C -> T | Yes |
| 1291 | T -> G | No |
| 1323 | A -> C | Yes |
| 1532 | G -> C | Yes |
| 1769 | C -> A | Yes |
| 1781 | C -> | No |
| 1788 | G -> A | Yes |
| 1790 | G -> T | Yes |
| 1882 | C -> | No |
| 1882 | C -> G | No |
| 1904 | C -> T | Yes |
| 1913 | C -> T | Yes |
| 1920 | A -> G | No |
| 1934 | G -> A | No |
| 2010 | G -> A | Yes |
| 2068 | C -> | No |
| 2068 | C -> G | No |
| 2122 | C -> T | Yes |
| 2162 | C -> T | Yes |
| 2188 | C -> | No |
| 2188 | C -> A | No |
| 2198 | G -> | No |
| 2252 | C -> | No |
| 2472 | A -> G | No |
| 2629 | C -> | No |
| 2718 | G -> T | No |
| 2773 | C -> | No |
| 2791 | G -> | No |
| 3100 | C -> | No |
| 3386 | T -> | No |
| 3386 | T -> C | No |
| 3513 | T -> A | No |
| 3520 | C -> T | No |
| 3636 | -> T | No |
| 3736 | -> G | No |
| 3736 | -> T | No |
| 3834 | G -> A | No |
| 3966 | T -> G | No |

Variant protein HUMICAMA1A_PEA_{—}1_P15 (SEQ ID NO:312) according to the present invention has an amino acid sequence as given at the end of the application; it is encoded by transcript(s) HUMICAMA1A_PEA_{—}1_T4 (SEQ ID NO:280). An alignment is given to the known protein (Intercellular adhesion molecule-1 precursor (SEQ ID NO:307)) at the end of the application. One or more alignments to one or more previously published protein sequences are given at the end of the application. A brief description of the relationship of the variant protein according to the present invention to each such aligned protein is as follows:

Comparison report between HUMICAMA1A_PEA_{—}1_P15 (SEQ ID NO:312) and ICA1_HUMAN (SEQ ID NO:307):

1. An isolated chimeric polypeptide encoding for HUMICAMA1A_PEA_{—}1_P15 (SEQ ID NO:312), comprising a first amino acid sequence being at least 90% homologous to MAPSSPRPALPALLVLLGALFPGPGNAQTSVSPSKVILPRGGSVLVTCSTSCDQPKLLGIE TPLPKKELLLPGNNRKVYELSNVQEDSQPMCYSNCPDGQSTAKTFLTVYWTPERVELA PLPSWQPVGKNLTLRCQVEGGAPRANLTVVLLRGEKELKREPAVGEPAEVTTTVLVRR DHHGANFSCRTELDLRPQGLELFENTSAPYQLQTF corresponding to amino acids 1-212 of ICA1_HUMAN (SEQ ID NO:307), which also corresponds to amino acids 1-212 of HUMICAMA1A_PEA_{—}1_P15 (SEQ ID NO:312), and a second amino acid sequence being at least 70%, optionally at least 80%, preferably at least 85%, more preferably at least 90% and most preferably at least 95% homologous to a polypeptide having the sequence GED corresponding to amino acids 213-215 of HUMICAMA1A_PEA_{—}1_P15 (SEQ ID NO:312), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMICAMA1A_PEA_{—}1_P15 (SEQ ID NO:312) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein HUMICAMA1A_PEA_{—}1_P15 (SEQ ID NO:312) Sequence provides support for the deduced sequence of this variant protein according to the present invention).

TABLE 16
Amino acid mutations

| SNP position(s) on | | |
|---|---|---|
| amino acid sequence | Alternative amino acid(s) | Previously known SNP? |
| 56 | K -> M | Yes |
| 155 | K -> N | Yes |

The glycosylation sites of variant protein HUMICAMA1A_PEA_{—}1_P15 (SEQ ID NO:312), as compared to the known protein Intercellular adhesion molecule-1 precursor (SEQ ID NO:307), are described in Table 17 (given according to their position(s) on the amino acid sequence in the first column; the second column indicates whether the glycosylation site is present in the variant protein; and the last column indicates whether the position is different on the variant protein).

TABLE 17
Glycosylation site(s)

| Position(s) on known | Present | |
|---|---|---|
| amino acid sequence | in variant protein? | Position in variant protein? |
| 385 | no | |
| 296 | no | |
| 202 | yes | 202 |
| 145 | yes | 145 |
| 130 | yes | 130 |
| 406 | no | |
| 183 | yes | 183 |
| 267 | no | |

Variant protein HUMICAMA1A_PEA_{—}1_P15 (SEQ ID NO: 312) is encoded by the following transcript(s): HUMICAMA1A_PEA_{—}1_T4 (SEQ ID NO:280), for which the sequence(s) is/are given at the end of the application. The coding portion of transcript HUMICAMA1A_PEA_{—}1_T4 (SEQ ID NO:280) is shown in bold; this coding portion starts at position 1332 and ends at position 1976. The transcript also has the following SNPs as listed in Table 18 (given according to their position on the nucleotide sequence, with the alternative nucleic acid listed; the last column indicates whether the SNP is known or not; the presence of known SNPs in variant protein HUMICAMA1A_PEA_{—}1_P15 (SEQ ID NO:312) Sequence provides support for the deduced sequence of this variant protein according to the present invention).

TABLE 18
Nucleic acid SNPs

| SNP position on | | |
|---|---|---|
| nucleotide sequence | Alternative nucleic acid | Previously known SNP? |
| 1 | G -> C | No |
| 169 | G -> A | Yes |
| 490 | A -> | Yes |
| 1288 | C -> T | Yes |
| 1291 | T -> G | No |
| 1323 | A -> C | Yes |
| 1498 | A -> T | Yes |
| 1796 | G -> C | Yes |
| 2023 | C -> T | Yes |
| 2094 | A -> G | No |
| 2132 | T -> C | No |
| 2279 | C -> A | Yes |
| 2291 | C -> | No |
| 2298 | G -> A | Yes |
| 2300 | G -> T | Yes |
| 2392 | C -> | No |
| 2392 | C -> G | No |
| 2414 | C -> T | Yes |
| 2423 | C -> T | Yes |
| 2430 | A -> G | No |
| 2444 | G -> A | No |
| 2520 | G -> A | Yes |
| 2578 | C -> | No |
| 2578 | C -> G | No |
| 2632 | C -> T | Yes |
| 2672 | C -> T | Yes |
| 2698 | C -> | No |
| 2698 | C -> A | No |
| 2708 | G -> | No |
| 2762 | C -> | No |
| 2982 | A -> G | No |
| 3139 | C -> | No |
| 3228 | G -> T | No |
| 3283 | C -> | No |
| 3301 | G -> | No |
| 3610 | C -> | No |
| 3896 | T -> | No |
| 3896 | T -> C | No |
| 4023 | T -> A | No |
| 4030 | C -> T | No |
| 4146 | -> T | No |
| 4246 | -> G | No |
| 4246 | -> T | No |
| 4344 | G -> A | No |
| 4476 | T -> G | No |

As noted above, cluster HUMICAMA1A features 22 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMICAMA1A_PEA_node_{—}0 (SEQ ID NO:285) according to the present invention is supported by 50 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA-1-T12 (SEQ ID NO:283) and HUMICAMA1A_PEA_{—}1_T16 (SEQ ID NO:284). Table 19 below describes the starting and ending position of this segment on each transcript.

TABLE 19
Segment location on transcripts

| | Segment | |
|---|---|---|
| | starting | Segment |
| Transcript name | position | ending position |
| HUMICAMA1A_PEA_1_T2 (SEQ ID | 1 | 1398 |
| NO: 279) | | |
| HUMICAMA1A_PEA_1_T4 (SEQ ID | 1 | 1398 |
| NO: 280) | | |
| HUMICAMA1A_PEA_1_T5 (SEQ ID | 1 | 1398 |
| NO: 281) | | |
| HUMICAMA1A_PEA_1_T8 (SEQ ID | 1 | 1398 |
| NO: 282) | | |
| HUMICAMA1A_PEA_1_T12 (SEQ | 1 | 1398 |
| ID NO: 283) | | |
| HUMICAMA1A_PEA_1_T16 (SEQ | 1 | 1398 |
| ID NO: 284) | | |

Segment cluster HUMICAMA1A_PEA_{—}1_node_{—}3 (SEQ ID NO:286) according to the present invention is supported by 66 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T12 (SEQ ID NO:283) and HUMICAMA1A_PEA_{—}1_T16 (SEQ ID NO:284). Table 20 below describes the starting and ending position of this segment on each transcript.

TABLE 20
Segment location on transcripts

| | | Segment |
|---|---|---|
| | Segment | ending |
| Transcript name | starting position | position |
| HUMICAMA1A_PEA_1_T2 (SEQ ID | 1464 | 1620 |
| NO: 279) | | |
| HUMICAMA1A_PEA_1_T4 (SEQ ID | 1464 | 1620 |
| NO: 280) | | |
| HUMICAMA1A_PEA_1_T5 (SEQ ID | 1464 | 1620 |
| NO: 281) | | |
| HUMICAMA1A_PEA_1_T12 (SEQ | 1464 | 1620 |
| ID NO: 283) | | |
| HUMICAMA1A_PEA_1_T16 (SEQ | 1464 | 1620 |
| ID NO: 284) | | |

Segment cluster HUMICAMA1A_PEA_{—}1_node_{—}12 (SEQ ID NO:287) according to the present invention is supported by 87 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 21 below describes the starting and ending position of this segment on each transcript.

TABLE 21
Segment location on transcripts

| | | Segment |
|---|---|---|
| | Segment | ending |
| Transcript name | starting position | position |
| HUMICAMA1A_PEA_1_T2 (SEQ ID | 1663 | 1968 |
| NO: 279) | | |
| HUMICAMA1A_PEA_1_T4 (SEQ ID | 1663 | 1968 |
| NO: 280) | | |
| HUMICAMA1A_PEA_1_T5 (SEQ ID | 1663 | 1968 |
| NO: 281) | | |
| HUMICAMA1A_PEA_1_T8 (SEQ ID | 1399 | 1704 |
| NO: 282) | | |
| HUMICAMA1A_PEA_1_T12 (SEQ | 1663 | 1968 |
| ID NO: 283) | | |
| HUMICAMA1A_PEA_1_T16 (SEQ | 1663 | 1968 |
| ID NO: 284) | | |

Segment cluster HUMICAMA1A_PEA_node_{—}13 (SEQ ID NO:288) according to the present invention is supported by 3 libraries. The number of libraries was determined as previously described. This segment can be found in the following transcript(s): HUMICAMA1A_PEA_{—}1_T4 (SEQ ID NO:280). Table 22 below describes the starting and ending position of this segment on each transcript.

TABLE 22
Segment location on transcripts

| | | Segment |
|---|---|---|
| | Segment | ending |
| Transcript name | starting position | position |
| HUMICAMA1A_PEA_1_T4 (SEQ ID | 1969 | 2214 |
| NO: 280) | | |

Segment cluster HUMICAMA1A_PEA_{—}1_node_{—}14 (SEQ ID NO:289) according to the present invention is supported by 88 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 23 below describes the starting and ending position of this segment on each transcript.

TABLE 23
Segment location on transcripts

| | Segment | |
|---|---|---|
| | starting | Segment |
| Transcript name | position | ending position |
| HUMICAMA1A_PEA_1_T2 (SEQ ID | 1969 | 2256 |
| NO: 279) | | |
| HUMICAMA1A_PEA_1_T4 (SEQ ID | 2215 | 2502 |
| NO: 280) | | |
| HUMICAMA1A_PEA_1_T5 (SEQ ID | 1969 | 2256 |
| NO: 281) | | |
| HUMICAMA1A_PEA_1_T8 (SEQ ID | 1705 | 1992 |
| NO: 282) | | |
| HUMICAMA1A_PEA_1_T12 (SEQ | 1969 | 2256 |
| ID NO: 283) | | |
| HUMICAMA1A_PEA_1_T16 (SEQ | 1969 | 2256 |
| ID NO: 284) | | |

Segment cluster HUMICAMA1A_PEA_{—}1_node_{—}20 (SEQ ID NO:290) according to the present invention is supported by 7 libraries. The number of libraries was determined as previously described. This segment can be found in the following transcript(s): HUMICAMA1A_PEA_{—}1_T5 (SEQ ID NO:281), HUMICAMA1A_PEA_{—}1_T12 (SEQ ID NO:283) and HUMICAMA1A_PEA_{—}1_T16 (SEQ ID NO:284). Table 24 below describes the starting and ending position of this segment on each transcript.

TABLE 24
Segment location on transcripts

| | Segment | |
|---|---|---|
| | starting | Segment |
| Transcript name | position | ending position |
| HUMICAMA1A_PEA_1_T5 (SEQ ID | 2512 | 2636 |
| NO: 281) | | |
| HUMICAMA1A_PEA_1_T12 (SEQ | 2512 | 2636 |
| ID NO: 283) | | |
| HUMICAMA1A_PEA_1_T16 (SEQ | 2512 | 2636 |
| ID NO: 284) | | |

Segment cluster HUMICAMA1A_PEA_{—}1_node_{—}21 (SEQ ID NO:291) according to the present invention is supported by 91 libraries. The number of libraries was determined as previously described. This segment can be found in the following transcript(s): HUMICAMA1A_PEA-1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 25 below describes the starting and ending position of this segment on each transcript.

TABLE 25
Segment location on transcripts

| | Segment | |
|---|---|---|
| | starting | Segment |
| Transcript name | position | ending position |
| HUMICAMA1A_PEA_1_T2 (SEQ ID | 2594 | 2820 |
| NO: 279) | | |
| HUMICAMA1A_PEA_1_T4 (SEQ ID | 2758 | 2984 |
| NO: 280) | | |
| HUMICAMA1A_PEA_1_T5 (SEQ ID | 2637 | 2863 |
| NO: 281) | | |
| HUMICAMA1A_PEA_1_T8 (SEQ ID | 2248 | 2474 |
| NO: 282) | | |
| HUMICAMA1A_PEA_1_T12 (SEQ | 2637 | 2863 |
| ID NO: 283) | | |
| HUMICAMA1A_PEA_1_T16 (SEQ | 2637 | 2863 |
| ID NO: 284) | | |

Segment cluster HUMICAMA1A_PEA_{—}1_node_{—}24 (SEQ ID NO:292) according to the present invention is supported by 109 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_T16 (SEQ ID NO:284). Table 26 below describes the starting and ending position of this segment on each transcript.

TABLE 26
Segment location on transcripts

| | Segment | |
|---|---|---|
| | starting | Segment |
| Transcript name | position | ending position |
| HUMICAMA1A_PEA_1_T2 (SEQ ID | 2840 | 2986 |
| NO: 279) | | |
| HUMICAMA1A_PEA_1_T4 (SEQ ID | 3004 | 3150 |
| NO: 280) | | |
| HUMICAMA1A_PEA_1_T5 (SEQ ID | 2883 | 3029 |
| NO: 281) | | |
| HUMICAMA1A_PEA_1_T8 (SEQ ID | 2494 | 2640 |
| NO: 282) | | |
| HUMICAMA1A_PEA_1_T12 (SEQ | 2969 | 3115 |
| ID NO: 283) | | |
| HUMICAMA1A_PEA_1_T16 (SEQ | 2969 | 3115 |
| ID NO: 284) | | |

Segment cluster HUMICAMA1A_PEA_{—}1_node_{—}25 (SEQ ID NO:293) according to the present invention is supported by 108 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282) and HUMICAMA1A_PEA_{—}1_T12 (SEQ ID NO:283). Table 27 below describes the starting and ending position of this segment on each transcript.

TABLE 27
Segment location on transcripts

| | Segment | |
|---|---|---|
| | starting | Segment |
| Transcript name | position | ending position |
| HUMICAMA1A_PEA_1_T2 (SEQ ID | 2987 | 3118 |
| NO: 279) | | |
| HUMICAMA1A_PEA_1_T4 (SEQ ID | 3151 | 3282 |
| NO: 280) | | |
| HUMICAMA1A_PEA_1_T5 (SEQ ID | 3030 | 3161 |
| NO: 281) | | |
| HUMICAMA1A_PEA_1_T8 (SEQ ID | 2641 | 2772 |
| NO: 282) | | |
| HUMICAMA1A_PEA_1_T12 (SEQ | 3116 | 3247 |
| ID NO: 283) | | |

Segment cluster HUMICAMA1A_PEA_{—}1_node_{—}27 (SEQ ID NO:294) according to the present invention is supported by 225 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282) and HUMICAMA1A_PEA_{—}1_T12 (SEQ ID NO:283). Table 28 below describes the starting and ending position of this segment on each transcript.

TABLE 28
Segment location on transcripts

| | Segment | |
|---|---|---|
| | starting | Segment |
| Transcript name | position | ending position |
| HUMICAMA1A_PEA_1_T2 (SEQ ID | 3138 | 4204 |
| NO: 279) | | |
| HUMICAMA1A_PEA_1_T4 (SEQ ID | 3302 | 4368 |
| NO: 280) | | |
| HUMICAMA1A_PEA_1_T5 (SEQ ID | 3181 | 4247 |
| NO: 281) | | |
| HUMICAMA1A_PEA_1_T8 (SEQ ID | 2792 | 3858 |
| NO: 282) | | |
| HUMICAMA1A_PEA_1_T12 (SEQ | 3267 | 4333 |
| ID NO: 283) | | |

Segment cluster HUMICAMA1A_PEA_{—}1_node_{—}29 (SEQ ID NO:295) according to the present invention is supported by 53 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282) and HUMICAMA1A_PEA_{—}1_T12 (SEQ ID NO:283). Table 29 below describes the starting and ending position of this segment on each transcript.

TABLE 29
Segment location on transcripts

| | Segment | |
|---|---|---|
| | starting | Segment |
| Transcript name | position | ending position |
| HUMICAMA1A_PEA_1_T2 (SEQ ID | 4209 | 4341 |
| NO: 279) | | |
| HUMICAMA1A_PEA_1_T4 (SEQ ID | 4373 | 4505 |
| NO: 280) | | |
| HUMICAMA1A_PEA_1_T5 (SEQ ID | 4252 | 4384 |
| NO: 281) | | |
| HUMICAMA1A_PEA_1_T8 (SEQ ID | 3863 | 3995 |
| NO: 282) | | |
| HUMICAMA1A_PEA_1_T12 (SEQ | 4338 | 4470 |
| ID NO: 283) | | |

According to an optional embodiment of the present invention, short segments related to the above cluster are also provided. These segments are up to about 120 bp in length, and so are included in a separate description.

Segment cluster HUMICAMA1A_PEA_{—}1_node_{—}2 (SEQ ID NO:296) according to the present invention is supported by 58 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T12 (SEQ ID NO:283) and HUMICAMA1A_PEA_{—}1_T16 (SEQ ID NO:284). Table 30 below describes the starting and ending position of this segment on each transcript.

TABLE 30
Segment location on transcripts

| | Segment | |
|---|---|---|
| | starting | Segment |
| Transcript name | position | ending position |
| HUMICAMA1A_PEA_1_T2 (SEQ ID | 1399 | 1463 |
| NO: 279) | | |
| HUMICAMA1A_PEA_1_T4 (SEQ ID | 1399 | 1463 |
| NO: 280) | | |
| HUMICAMA1A_PEA_1_T5 (SEQ ID | 1399 | 1463 |
| NO: 281) | | |
| HUMICAMA1A_PEA_1_T12 (SEQ | 1399 | 1463 |
| ID NO: 283) | | |
| HUMICAMA1A_PEA_1_T16 (SEQ | 1399 | 1463 |
| ID NO: 284) | | |

Segment cluster HUMICAMA1A_PEA_{—}1_node_{—}4 (SEQ ID NO:297) according to the present invention is supported by 62 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T12 (SEQ ID NO:283) and HUMICAMA1A_PEA_{—}1_T16 (SEQ ID NO:284). Table 31 below describes the starting and ending position of this segment on each transcript.

TABLE 31
Segment location on transcripts

| | Segment | |
|---|---|---|
| | starting | Segment |
| Transcript name | position | ending position |
| HUMICAMA1A_PEA_1_T2 (SEQ ID | 1621 | 1662 |
| NO: 279) | | |
| HUMICAMA1A_PEA_1_T4 (SEQ ID | 1621 | 1662 |
| NO: 280) | | |
| HUMICAMA1A_PEA_1_T5 (SEQ ID | 1621 | 1662 |
| NO: 281) | | |
| HUMICAMA1A_PEA_1_T12 (SEQ | 1621 | 1662 |
| ID NO: 283) | | |
| HUMICAMA1A_PEA_1_T16 (SEQ | 1621 | 1662 |
| ID NO: 284) | | |

Segment cluster HUMICAMA1A_PEA_{—}1_node_{—}15 (SEQ ID NO:298) according to the present invention is supported by 4 libraries. The number of libraries was determined as previously described. This segment can be found in the following transcript(s): HUMICAMA1A_PEA_{—}1_T2 (SEQ ID NO:279). Table 32 below describes the starting and ending position of this segment on each transcript.

TABLE 32
Segment location on transcripts

| | Segment | |
|---|---|---|
| | starting | Segment |
| Transcript name | position | ending position |
| HUMICAMA1A_PEA_1_T2 (SEQ ID | 2257 | 2338 |
| NO: 279) | | |

Segment cluster HUMICAMA1A_PEA_{—}1_node_{—}16 (SEQ ID NO:299) according to the present invention is supported by 58 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 33 below describes the starting and ending position of this segment on each transcript.

TABLE 33
Segment location on transcripts

| | Segment | |
|---|---|---|
| | starting | Segment |
| Transcript name | position | ending position |
| HUMICAMA1A_PEA_1_T2 (SEQ ID | 2339 | 2457 |
| NO: 279) | | |
| HUMICAMA1A_PEA_1_T4 (SEQ ID | 2503 | 2621 |
| NO: 280) | | |
| HUMICAMA1A_PEA_1_T5 (SEQ ID | 2257 | 2375 |
| NO: 281) | | |
| HUMICAMA1A_PEA_1_T8 (SEQ ID | 1993 | 2111 |
| NO: 282) | | |
| HUMICAMA1A_PEA_1_T12 (SEQ | 2257 | 2375 |
| ID NO: 283) | | |
| HUMICAMA1A_PEA_1_T16 (SEQ | 2257 | 2375 |
| ID NO: 284) | | |

Segment cluster HUMICAMA1A_PEA_{—}1_node_{—}17 (SEQ ID NO:300) according to the present invention can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 34 below describes the starting and ending position of this segment on each transcript.

TABLE 34
Segment location on transcripts

| | Segment | |
|---|---|---|
| | starting | Segment |
| Transcript name | position | ending position |
| HUMICAMA1A_PEA_1_T2 (SEQ ID | 2458 | 2478 |
| NO: 279) | | |
| HUMICAMA1A_PEA_1_T4 (SEQ ID | 2622 | 2642 |
| NO: 280) | | |
| HUMICAMA1A_PEA_1_T5 (SEQ ID | 2376 | 2396 |
| NO: 281) | | |
| HUMICAMA1A_PEA_1_T8 (SEQ ID | 2112 | 2132 |
| NO: 282) | | |
| HUMICAMA1A_PEA_1_T12 (SEQ | 2376 | 2396 |
| ID NO: 283) | | |
| HUMICAMA1A_PEA_1_T16 (SEQ | 2376 | 2396 |
| ID NO: 284) | | |

Segment cluster HUMICAMA1A_PEA_{—}1_node_{—}18 (SEQ ID NO:301) according to the present invention is supported by 57 libraries. The number of libraries was determined as previously described. This segment can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 35 below describes the starting and ending position of this segment on each transcript.

TABLE 35
Segment location on transcripts

| | Segment | |
|---|---|---|
| | starting | Segment |
| Transcript name | position | ending position |
| HUMICAMA1A_PEA_1_T2 (SEQ ID | 2479 | 2568 |
| NO: 279) | | |
| HUMICAMA1A_PEA_1_T4 (SEQ ID | 2643 | 2732 |
| NO: 280) | | |
| HUMICAMA1A_PEA_1_T5 (SEQ ID | 2397 | 2486 |
| NO: 281) | | |
| HUMICAMA1A_PEA_1_T8 (SEQ ID | 2133 | 2222 |
| NO: 282) | | |
| HUMICAMA1A_PEA_1_T12 (SEQ | 2397 | 2486 |
| ID NO: 283) | | |
| HUMICAMA1A_PEA_1_T16 (SEQ | 2397 | 2486 |
| ID NO: 284) | | |

Segment cluster HUMICAMA1A_PEA_{—}1_node_{—}19 (SEQ ID NO:302) according to the present invention can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 36 below describes the starting and ending position of this segment on each transcript.

TABLE 36
Segment location on transcripts

| | Segment | |
|---|---|---|
| | starting | Segment |
| Transcript name | position | ending position |
| HUMICAMA1A_PEA_1_T2 (SEQ ID | 2569 | 2593 |
| NO: 279) | | |
| HUMICAMA1A_PEA_1_T4 (SEQ ID | 2733 | 2757 |
| NO: 280) | | |
| HUMICAMA1A_PEA_1_T5 (SEQ ID | 2487 | 2511 |
| NO: 281) | | |
| HUMICAMA1A_PEA_1_T8 (SEQ ID | 2223 | 2247 |
| NO: 282) | | |
| HUMICAMA1A_PEA_1_T12 (SEQ | 2487 | 2511 |
| ID NO: 283) | | |
| HUMICAMA1A_PEA_1_T16 (SEQ | 2487 | 2511 |
| ID NO: 284) | | |

Segment cluster HUMICAMA1A_PEA_node_{—}22 (SEQ ID NO:303) according to the present invention can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282), HUMICAMA1A_PEA_{—}1_T12 (SEQ ID NO:283) and HUMICAMA1A_PEA_{—}1_T16 (SEQ ID NO:284). Table 37 below describes the starting and ending position of this segment on each transcript.

TABLE 37
Segment location on transcripts

| | Segment | |
|---|---|---|
| | starting | Segment |
| Transcript name | position | ending position |
| HUMICAMA1A_PEA_1_T2 (SEQ ID | 2821 | 2839 |
| NO: 279) | | |
| HUMICAMA1A_PEA_1_T4 (SEQ ID | 2985 | 3003 |
| NO: 280) | | |
| HUMICAMA1A_PEA_1_T5 (SEQ ID | 2864 | 2882 |
| NO: 281) | | |
| HUMICAMA1A_PEA_1_T8 (SEQ ID | 2475 | 2493 |
| NO: 282) | | |
| HUMICAMA1A_PEA_1_T12 (SEQ | 2864 | 2882 |
| ID NO: 283) | | |
| HUMICAMA1A_PEA_1_T16 (SEQ | 2864 | 2882 |
| ID NO: 284) | | |

Segment cluster HUMICAMA1A_PEA_{—}1_node_{—}23 (SEQ ID NO:304) according to the present invention is supported by 5 libraries. The number of libraries was determined as previously described. This segment can be found in the following transcript(s): HUMICAMA1A_PEA_{—}1_T12 (SEQ ID NO:283) and HUMICAMA1A_PEA_{—}1_T16 (SEQ ID NO:284). Table 38 below describes the starting and ending position of this segment on each transcript.

TABLE 38
Segment location on transcripts

| | Segment | |
|---|---|---|
| | starting | Segment |
| Transcript name | position | ending position |
| HUMICAMA1A_PEA_1_T12 (SEQ | 2883 | 2968 |
| ID NO: 283) | | |
| HUMICAMA1A_PEA_1_T16 (SEQ | 2883 | 2968 |
| ID NO: 284) | | |

Segment cluster HUMICAMA1A_PEA_{—}1_node_{—}26 (SEQ ID NO:305) according to the present invention can be found in the following transcript(s): HUMICAMA1A_PEA_{—}1_T2 (SEQ ID NO:279), HUMICAMA1A_PEA_{—}1_T4 (SEQ ID NO:280), HUMICAMA1A_PEA_{—}1_T5 (SEQ ID NO:281), HUMICAMA1A_PEA_{—}1_T8 (SEQ ID NO:282) and HUMICAMA1A_PEA_{—}1_T12 (SEQ ID NO:283). Table 39 below describes the starting and ending position of this segment on each transcript.

TABLE 39
Segment location on transcripts

| | Segment | |
|---|---|---|
| | starting | Segment |
| Transcript name | position | ending position |
| HUMICAMA1A_PEA_1_T2 (SEQ ID | 3119 | 3137 |
| NO: 279) | | |
| HUMICAMA1A_PEA_1_T4 (SEQ ID | 3283 | 3301 |
| NO: 280) | | |
| HUMICAMA1A_PEA_1_T5 (SEQ ID | 3162 | 3180 |
| NO: 281) | | |
| HUMICAMA1A_PEA_1_T8 (SEQ ID | 2773 | 2791 |
| NO: 282) | | |
| HUMICAMA1A_PEA_1_T12 (SEQ | 3248 | 3266 |
| ID NO: 283) | | |

Segment cluster HUMICAMA1A_PEA_{—}1_node_{—}28 (SEQ ID NO:306) according to the present invention can be found in the following transcript(s): HUMICAMA1A_PEA_{—}1_T2 (SEQ ID NO:279), HUMICAMA1A_PEA_T4 (SEQ ID NO:280), HUMICAMA1A_PEA_{—}1_T5 (SEQ ID NO:281), HUMICAMA1A_PEA_{—}1_T8 (SEQ ID NO:282) and HUMICAMA1A_PEA_{—}1_T12 (SEQ ID NO:283). Table 40 below describes the starting and ending position of this segment on each transcript.

TABLE 40
Segment location on transcripts

| | Segment | |
|---|---|---|
| | starting | Segment |
| Transcript name | position | ending position |
| HUMICAMA1A_PEA_1_T2 (SEQ ID | 4205 | 4208 |
| NO: 279) | | |
| HUMICAMA1A_PEA_1_T4 (SEQ ID | 4369 | 4372 |
| NO: 280) | | |
| HUMICAMA1A_PEA_1_T5 (SEQ ID | 4248 | 4251 |
| NO: 281) | | |
| HUMICAMA1A_PEA_1_T8 (SEQ ID | 3859 | 3862 |
| NO: 282) | | |
| HUMICAMA1A_PEA_1_T12 (SEQ | 4334 | 4337 |
| ID NO: 283) | | |

Variant protein alignment to the previously known protein:

| Sequence name: ICA1_HUMAN (SEQ ID NO:307) | | | |
|---|---|---|---|
| Sequence documentation: | | | |
| Alignment of: HUMICAMA1A_PEA_1_P2 (SEQ ID NO:309) × | | | |
| ICA1_HUMAN (SEQ ID NO:307) . . | | | |
| Alignment segment 1/1: | | | |
| Quality: | 2994.00 | Escore: | 0 |
| Matching length: | 309 | Total length: | 309 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MAPSSPRPALPALLVLLGALFPGPGNAQTSVSPSKVILPRGGSVLVTCST | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MAPSSPRPALPALLVLLGALFPGPGNAQTSVSPSKVILPRGGSvLVTCST | 50 |
| | . . . . . | |
| 51 | SCDQPKLLGIETPLPKKELLLPGNNRKVYELSNVQEDSQPMCYSNCPDGQ | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SCDQPKLLGIETPLPKKELLLPGNNRKVYELSNVQEDSQPNCYSNCPDGQ | 100 |
| | . . . . . | |
| 101 | STAKTFLTVYWTPERVELAPLPSWQPVGRNLTLRCQVEGGAPRANLTVVL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | STAKTFLTVYWTPERVELAPLPSWQPVGKNLTLRCQVEGGAPRANLTVVL | 150 |
| | . . . . . | |
| 151 | LRGEKELKREPAVGEPAEVTTTVLVRRDHHGANFSCRTELDLRPQGLELF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | LRGEKELKREPAVGEPAEVTTTVLVRRDHHGANFSCRTELDLRPQGLELF | 200 |
| | . . . . . | |
| 201 | ENTSAPYQLQTFVLPATPPQLVSPRVLEVDTQGTVVCSLDGLFPVSEAQV | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | ENTSAPYQLQTFVLPATPPQLVSPRVLEVDTQGTVVCSLDGLFPVSEAQV | 250 |
| | . . . . . | |
| 251 | HLALGDQRLNPTVTYGNDSFSAKASVSVTAEDEGTQRLTCAVILGNQSQE | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | HLALGDQRLNPTVTYGNDSFSAKASVSVTAEDEGTQRLTCAVILGNQSQE | 300 |
| 301 | TLQTVTIYS | 309 |
| | \|\|\|\|\|\|\|\|\| | |
| 301 | TLQTVTIYS | 309 |

| Sequence name: ICA1_HUMAN (SEQ ID NO:307) | | | |
|---|---|---|---|
| Sequence documentation: | | | |
| Alignment of: HUMICAMA1A_PEA_1_P5 (SEQ ID NO:310) × | | | |
| ICA1_HUMAN (SEQ ID NO:307) . . | | | |
| Alignment segment 1/1: | | | |
| Quality: | 3802.00 | Escore: | 0 |
| Matching length: | 393 | Total length: | 393 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MAPSSPRPALPALLVLLGALFPGPGNAQTSVSPSKVILPRGGSVLVTCST | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MAPSSPRPALPALLVLLGALFPGPGNAQTSVSPSKVILPRGGSVLVTCST | 50 |
| | . . . . . | |
| 51 | SCDQPKLLGIETPLPKKELLLPGNNRKVYELSNVQEDSQPMCYSNCPDGQ | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SCDQPKLLGIETPLPKKELLLPGNNRKVYELSNVQEDSQPMCYSNCPDGQ | 100 |
| | . . . . . | |
| 101 | STAKTFLTVYWTPERVELAPLPSWQPVGKNLTLRCQVEGGAPRANLTVVL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | STAKTFLTVYWTPERVELAPLPSWQPVGKNLTLRCQVEGGAPRANLTVVL | 150 |
| | . . . . . | |
| 151 | LRGEKELKREPAVGEPAEVTTTVLVRRDHHGANFSCRTELDLRPQGLELF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | LRGEKELKREPAVGEPAEVTTTVLVRRDHHGANFSCRTELDLRPQGLELF | 200 |
| | . . . . . | |
| 201 | ENTSAPYQLQTFVLPATPPQLVSPRVLEVDTQGTVVCSLDGLFPVSEAQV | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | ENTSAPYQLQTFVLPATPPQLVSPRVLEVDTQGTVVCSLDGLFPVSEAQV | 250 |
| | . . . . . | |
| 251 | HLALGDQRLNPTVTYGNDSFSAKASVSVTAEDEGTQRLTCAVILGNQSQE | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | HLALGDQRLNPTVTYGNDSFSAKASVSVTAEDEGTQRLTCAVILGNQSQE | 300 |
| | . . . . . | |
| 301 | TLQTVTIYSFPAPNVILTKPEVSEGTEVTVKCEAHPRAKVTLNGVPAQPL | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | TLQTVTIYSFPAPNVILTKPEVSEGTEVTVKCEAHPRAKVTLNGVPAQPL | 350 |
| | . . . . | |
| 351 | GPRAQLLLKATPEDNGRSFSCSATLEVAGQLIHKNQTRELRVL | 393 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPRAQLLLKATPEDNGRSFSCSATLEVAGQLIHKNQTRELRVL | 393 |

| Sequence name: ICA1_HUMAN_V1 (SEQ ID NO:308) | | | |
|---|---|---|---|
| Sequence documentation: | | | |
| Alignment of: HUMICAMA1A_PEA_1_P8 (SEQ ID NO:311) × | | | |
| ICA1_HUMAN_V1 (SEQ ID NO:308) . . | | | |
| Alignment segment 1/1: | | | |
| Quality: | 4214.00 | Escore: | 0 |
| Matching length: | 444 | Total length: | 532 |
| Matching Percent | 100.00 | Matching Percent | 100.00 |
| Similarity: | | Identity: | |
| Total Percent | 83.46 | Total Percent | 83.46 |
| Similarity: | | Identity: | |
| Gaps: | 1 | | |

| Alignment: | | |
|---|---|---|
| | . . . . . | |
| 1 | MAPSSPRPALPALLVLLGALFPG........................... | 23 |
| | \|\|\|\|\|\|\|\|\|\|\|\|\|\|\|\|\|\|\|\|\|\| | |
| 1 | MAPSSPRPALPALLVLLGALFPGPGNAQTSVSPSKVILPRGGSVLVTCST | 50 |
| | . . . . . | |
| 23 | .................................................. | 23 |
| 51 | SCDQPKLLGIETPLPKKELLLPGNNRKVYELSNVQEDSQPMCYSNCPDGQ | 100 |
| | . . . . . | |
| 24 | ...........TPERVELAPLPSWQPVGKNLTLRCQVEGGAPRANLTVVL | 62 |
| | \|\|\|\|\|\|\|\|\|\|\|\|\|\|\|\|\|\|\|\|\|\|\|\|\|\|\|\|\|\|\|\|\|\|\|\|\|\|\| | |
| 101 | STAKTFLTVYWTPERVELAPLPSWQPVGKNLTLRCQVEGGAPRANLTVVL | 150 |
| | . . . . . | |
| 63 | LRGEKELKREPAVGEPAEVTTTVLVRRDHHGANFSCRTELDLRPQGLELF | 112 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | LRGEKELKREPAVGEPAEVTTTVLVRRDHHGANFSCRTELDLRPQGLELF | 200 |
| | . . . . . | |
| 113 | ENTSAPYQLQTFVLPATPPQLVSPRVLEVDTQGTVVCSLDGLFPVSEAQV | 162 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | ENTSAPYQLQTFVLPATPPQLVSPRVLEVDTQGTVVCSLDGLFPVSEAQV | 250 |
| | . . . . . | |
| 163 | HLALGDQRLNPTVTYGNDSFSAKASVSVTAEDEGTQRLTCAVILGNQSQE | 212 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | HLALGDQRLNPTVTYGNDSFSAKASVSVTAEDEGTQRLTCAVILGNQSQE | 300 |
| | . . . . . | |
| 213 | TLQTVTIYSFPAPNVILTKPEVSEGTEVTVKCEAHPRAKVTLNGVPAQPL | 262 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | TLQTVTIYSFPAPNVILTKPEVSEGTEVTVKCEAHPRAKVTLNGVPAQPL | 350 |
| | . . . . . | |
| 263 | GPRAQLLLKATPEDNGRSFSCSATLEVAGQLIHKNQTRELRVLYGPRLDE | 312 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPRAQLLLKATPEDNGRSFSCSATLEVAGQLIHKNQTRELRVLYGPRLDE | 400 |
| | . . . . . | |
| 313 | RDCPGNWTWPENSQQTPMCQAWGNPLPELKCLKDGTFPLPIGESVTVTRD | 362 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | RDCPGNWTWPENSQQTPMCQAWGNPLPELKCLKDGTFPLPIGESVTVTRD | 450 |
| | . . . . . | |
| 363 | LEGTYLCRARSTQGEVTRKVTVNVLSPRYEIVIITVVAAAVIMGTAGLST | 412 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | LEGTYLCRARSTQGEVTRKVTVNVLSPRYEIVIITVVAAAVIMGTAGLST | 500 |
| | . . . | |
| 413 | YLYNRQRKIKKYRLQQAQKGTPMKPNTQATPP | 444 |
| | \|\|\|\|\|\|\|\|\|\|\|\|\|\|\|\|\|\|\|\|\|\|\|\|\|\|\|\|\|\|\|\| | |
| 501 | YLYNRQRKIKKYRLQQAQKGTPMKPNTQATPP | 532 |

| Sequence name: ICA1_HUMAN (SEQ ID NO:307) | | | |
|---|---|---|---|
| Sequence documentation: | | | |
| Alignment of: HUMICAMA1A_PEA_1_P15 (SEQ ID NO:312) × | | | |
| ICA1_HUMAN (SEQ ID NO:307) . . | | | |
| Alignment segment 1/1: | | | |
| Quality: | 2076.00 | Escore: | 0 |
| Matching length: | 212 | Total length: | 212 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MAPSSPRPALPALLVLLGALFPGPGNAQTSVSPSKVILPRGGSVLVTCST | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MAPSSPRPALPALLVLLGALFPGPGNAQTSVSPSKVILPRGGSVLVTCST | 50 |
| | . . . . . | |
| 51 | SCDQPKLLGIETPLPKKELLLPGNNRKVYELSNVQEDSQPMCYSNCPDGQ | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SCDQPKLLGIETPLPKKELLLPGNNRKVYELSNVQEDSQPMCYSNCPDGQ | 100 |
| | . . . . . | |
| 101 | STAKTFLTVYWTPERVELAPLPSWQPVGKNLTLRCQVEGGAPRANLTVVL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | STAKTFLTVYWTPERVELAPLPSWQFVGKNLTLRCQVEGGAPRANLTVVL | 150 |
| | . . . . . | |
| 151 | LRGEKELKREPAVGEPAEVTTTVLVRRDHHGANFSCRTELDLRPQGLELF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | LRGEKELKREPAVGEPAEVTTTVLVRRDHHGANFSCRTELDLRPQGLELF | 200 |
| | . | |
| 201 | ENTSAPYQLQTF | 212 |
| | \|\|\|\|\|\|\|\|\|\|\|\| | |
| 201 | ENTSAPYQLQTF | 212 |

### Description for Cluster HUMLYSYL

Cluster HUMLYSYL features 10 transcript(s) and 44 segment(s) of interest, the names for which are given in Tables 1 and 2, respectively, the sequences themselves are given at the end of the application. The selected protein variants are given in table 3.

TABLE 1
Transcripts of interest

| Transcript Name | Sequence ID No. |
|---|---|
| HUMLYSYL_PEA_1_T2 | 313 |
| HUMLYSYL_PEA_1_T4 | 314 |
| HUMLYSYL_PEA_1_T5 | 315 |
| HUMLYSYL_PEA_1_T6 | 316 |
| HUMLYSYL_PEA_1_T8 | 317 |
| HUMLYSYL_PEA_1_T9 | 318 |
| HUMLYSYL_PEA_1_T19 | 319 |
| HUMLYSYL_PEA_1_T20 | 320 |
| HUMLYSYL_PEA_1_T22 | 321 |
| HUMLYSYL_PEA_1_T24 | 322 |

TABLE 2
Segments of interest

| Segment Name | Sequence ID No. |
|---|---|
| HUMLYSYL_PEA_1_node_6 | 323 |
| HUMLYSYL_PEA_1_node_14 | 324 |
| HUMLYSYL_PEA_1_node_19 | 325 |
| HUMLYSYL_PEA_1_node_38 | 326 |
| HUMLYSYL_PEA_1_node_55 | 327 |
| HUMLYSYL_PEA_1_node_59 | 328 |
| HUMLYSYL_PEA_1_node_61 | 329 |
| HUMLYSYL_PEA_1_node_62 | 330 |
| HUMLYSYL_PEA_1_node_65 | 331 |
| HUMLYSYL_PEA_1_node_71 | 332 |
| HUMLYSYL_PEA_1_node_72 | 333 |
| HUMLYSYL_PEA_1_node_3 | 334 |
| HUMLYSYL_PEA_1_node_4 | 335 |
| HUMLYSYL_PEA_1_node_8 | 336 |
| HUMLYSYL_PEA_1_node_10 | 337 |
| HUMLYSYL_PEA_1_node_11 | 338 |
| HUMLYSYL_PEA_1_node_12 | 339 |
| HUMLYSYL_PEA_1_node_16 | 340 |
| HUMLYSYL_PEA_1_node_20 | 341 |
| HUMLYSYL_PEA_1_node_23 | 342 |
| HUMLYSYL_PEA_1_node_25 | 343 |
| HUMLYSYL_PEA_1_node_28 | 344 |
| HUMLYSYL_PEA_1_node_30 | 345 |
| HUMLYSYL_PEA_1_node_31 | 346 |
| HUMLYSYL_PEA_1_node_33 | 347 |
| HUMLYSYL_PEA_1_node_34 | 348 |
| HUMLYSYL_PEA_1_node_36 | 349 |
| HUMLYSYL_PEA_1_node_40 | 350 |
| HUMLYSYL_PEA_1_node_41 | 351 |
| HUMLYSYL_PEA_1_node_42 | 352 |
| HUMLYSYL_PEA_1_node_44 | 353 |
| HUMLYSYL_PEA_1_node_45 | 354 |
| HUMLYSYL_PEA_1_node_46 | 355 |
| HUMLYSYL_PEA_1_node_48 | 356 |
| HUMLYSYL_PEA_1_node_49 | 357 |
| HUMLYSYL_PEA_1_node_52 | 358 |
| HUMLYSYL_PEA_1_node_53 | 359 |
| HUMLYSYL_PEA_1_node_56 | 360 |
| HUMLYSYL_PEA_1_node_63 | 361 |
| HUMLYSYL_PEA_1_node_64 | 362 |
| HUMLYSYL_PEA_1_node_66 | 363 |
| HUMLYSYL_PEA_1_node_67 | 364 |
| HUMLYSYL_PEA_1_node_68 | 365 |
| HUMLYSYL_PEA_1_node_70 | 366 |

TABLE 3
Proteins of interest

| | Sequence | |
|---|---|---|
| Protein Name | ID No. | Corresponding Transcript(s) |
| HUMLYSYL_PEA_1_P2 | 369 | HUMLYSYL_PEA_1_T2 |
| | | (SEQ ID NO: 313) |
| HUMLYSYL_PEA_1_P4 | 370 | HUMLYSYL_PEA_1_T4 |
| | | (SEQ ID NO: 314) |
| HUMLYSYL_PEA_1_P5 | 371 | HUMLYSYL_PEA_1_T5 |
| | | (SEQ ID NO: 315) |
| HUMLYSYL_PEA_1_P6 | 372 | HUMLYSYL_PEA_1_T6 |
| | | (SEQ ID NO: 316) |
| HUMLYSYL_PEA_1_P7 | 373 | HUMLYSYL_PEA_1_T9 |
| | | (SEQ ID NO: 318) |
| HUMLYSYL_PEA_1_P13 | 374 | HUMLYSYL_PEA_1_T19 |
| | | (SEQ ID NO: 319) |
| HUMLYSYL_PEA_1_P14 | 375 | HUMLYSYL_PEA_1_T20 |
| | | (SEQ ID NO: 320) |
| HUMLYSYL_PEA_1_P16 | 376 | HUMLYSYL_PEA_1_T22 |
| | | (SEQ ID NO: 321) |
| HUMLYSYL_PEA_1_P18 | 377 | HUMLYSYL_PEA_1_T24 |
| | | (SEQ ID NO: 322) |
| HUMLYSYL_PEA_1_P24 | 378 | HUMLYSYL_PEA_1_T8 |
| | | (SEQ ID NO: 317) |

These sequences are variants of the known protein Procollagen-lysine,2-oxoglutarate 5-dioxygenase 1 precursor (SEQ ID NO:367) (SwissProt accession identifier PLO1_HUMAN; known also according to the synonyms EC 1.14.11.4; Lysyl hydroxylase 1; LH1), referred to herein as the previously known protein.

Protein Procollagen-lysine,2-oxoglutarate 5-dioxygenase 1 precursor (SEQ ID NO:367) is known or believed to have the following function(s): forms hydroxylysine residues in -Xaa-Lys-Gly- sequences in collagens. These hydroxylysines serve as sites of attachment for carbohydrate units and are essential for the stability of the intermolecular collagen crosslinks. The sequence for protein Procollagen-lysine,2-oxoglutarate 5-dioxygenase 1 precursor is given at the end of the application, as “Procollagen-lysine,2-oxoglutarate 5-dioxygenase 1 precursor amino acid sequence” (SEQ ID NO:367). Known polymorphisms for this sequence are as shown in Table 4.

TABLE 4
Amino acid mutations for Known Protein

| SNP position(s) on | |
|---|---|
| amino acid sequence | Comment |
| 99 | T -> A. /FTId = VAR_014220. |
| 367-371 | Missing (in EDS-VI). /FTId = VAR_009269. |
| 532 | Missing (in EDS-VI). /FTId = VAR_006354. |
| 612 | W -> C (in EDS-VI). /FTId = VAR_006355. |
| 678 | G -> R (in EDS-VI). /FTId = VAR_006356. |
| 120 | A -> S |

Protein Procollagen-lysine,2-oxoglutarate 5-dioxygenase 1 precursor localization is believed to be Membrane bound in cisternae of rough endoplasmic reticulum.

The known protein was shown to be related to endometriosis (Yang et al, Best Pract Res Clin Obstet Gynaecol. 2004 April; 18(2):305-18). Variants of this cluster are suitable as diagnostic markers for endometriosis.

The following GO Annotation(s) apply to the previously known protein. The following annotation(s) were found: protein modification; epidermal differentiation, which are annotation(s) related to Biological Process; electron transporter; procollagen-lysine 5-dioxygenase; oxidoreductase; oxidoreductase, acting on single donors with incorporation of molecular oxygen, incorporation of two atoms of oxygen, which are annotation(s) related to Molecular Function; and endoplasmic reticulum; membrane, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

As noted above, cluster HUMLYSYL features 10 transcript(s), which were listed in Table 1 above. These transcript(s) encode for protein(s) which are variant(s) of protein Procollagen-lysine,2-oxoglutarate 5-dioxygenase 1 precursor (SEQ ID NO:367). A description of each variant protein according to the present invention is now provided.

Variant protein HUMLYSYL_PEA_{—}1_P2 (SEQ ID NO:369) according to the present invention has an amino acid sequence as given at the end of the application; it is encoded by transcript(s) HUMLYSYL_PEA_{—}1_T2 (SEQ ID NO:313). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P2 (SEQ ID NO:369) and PLO1_HUMAN-V1 (SEQ ID NO:368):

1. An isolated chimeric polypeptide encoding for HUMLYSYL_PEA_{—}1_P2 (SEQ ID NO:369), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQ corresponding to amino acids 1-490 of PLO1_HUMAN_V1 (SEQ ID NO:368), which also corresponds to amino acids 1-490 of HUMLYSYL_PEA_{—}1_P2 (SEQ ID NO:369), and a second amino acid sequence being at least 70%, optionally at least 80%, preferably at least 85%, more preferably at least 90% and most preferably at least 95% homologous to a polypeptide having the sequence VSQERAAQDALWMGQAGRMCSCS (SEQ ID NO:474) corresponding to amino acids 491-513 of HUMLYSYL_PEA_{—}1_P2 (SEQ ID NO:369), wherein said first amino acid sequence and second amino acid sequence are contiguous and in a sequential order.

2. An isolated polypeptide encoding for a tail of HUMLYSYL_PEA_{—}1_P2 (SEQ ID NO:369), comprising a polypeptide being at least 70%, optionally at least about 80%, preferably at least about 85%, more preferably at least about 90% and most preferably at least about 95% homologous to the sequence VSQERAAQDALWMGQAGRMCSCS (SEQ ID NO:474) in HUMLYSYL_PEA_{—}1_P2 (SEQ ID NO:369).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 5
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P2 (SEQ ID NO:369) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HUMLYSYL_PEA_{—}1_P2 (SEQ ID NO:369) Sequence provides support for the deduced sequence of this variant protein according to the present invention).

TABLE 6
Amino acid mutations

| SNP position(s) on amino acid | Alternative | Previously |
|---|---|---|
| sequence | amino acid(s) | known SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 232 | A -> G | No |
| 232 | A -> | No |
| 310 | R -> W | Yes |
| 381 | V -> M | Yes |
| 386 | A -> | No |

Variant protein HUMLYSYL_PEA_{—}1_P2 (SEQ ID NO:369) is encoded by the following transcript(s): HUMLYSYL_PEA_{—}1_T2 (SEQ ID NO:313), for which the sequence(s) is/are given at the end of the application. The coding portion of transcript HUMLYSYL_PEA_{—}1_T2 (SEQ ID NO:313) is shown in bold; this coding portion starts at position 104 and ends at position 1642. The transcript also has the following SNPs as listed in Table 7 (given according to their position on the nucleotide sequence, with the alternative nucleic acid listed; the last column indicates whether the SNP is known or not; the presence of known SNPs in variant protein HUMLYSYL_PEA_{—}1_P2 (SEQ ID NO:369) Sequence provides support for the deduced sequence of this variant protein according to the present invention).

TABLE 7
Nucleic acid SNPs

| SNP position | | |
|---|---|---|
| on nucleotide | Alternative | Previously |
| sequence | nucleic acid | known SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 798 | C -> | No |
| 798 | C -> G | No |
| 1031 | C -> T | Yes |
| 1244 | G -> A | Yes |
| 1260 | C -> | No |
| 1309 | C -> T | Yes |
| 1489 | G -> C | No |
| 1788 | A -> C | Yes |
| 2057 | G -> | No |
| 2088 | C -> T | Yes |
| 2094 | G -> C | Yes |
| 2118 | G -> T | Yes |
| 2280 | T -> C | Yes |
| 2289 | C -> G | Yes |
| 2300 | G -> | No |
| 2306 | C -> | No |
| 2404 | G -> | No |
| 2411 | C -> G | Yes |
| 2417 | C -> | No |
| 2541 | C -> | No |
| 2541 | C -> T | No |
| 2561 | C -> | No |
| 2598 | G -> A | No |
| 2637 | C -> | No |
| 2637 | C -> G | No |
| 2651 | C -> T | No |
| 2724 | G -> A | No |
| 2724 | G -> C | No |
| 2764 | G -> | No |
| 2771 | C -> T | Yes |
| 2780 | G -> C | Yes |
| 2873 | C -> | No |
| 2887 | G -> C | Yes |
| 2939 | C -> T | Yes |
| 2954 | G -> T | Yes |
| 3010 | C -> A | Yes |

Variant protein HUMLYSYL_PEA_{—}1_P4 (SEQ ID NO:370) according to the present invention has an amino acid sequence as given at the end of the application; it is encoded by transcript(s) HUMLYSYL_PEA_{—}1_T4 (SEQ ID NO:314). An alignment is given to the known protein (Procollagen-lysine,2-oxoglutarate 5-dioxygenase 1 precursor (SEQ ID NO:367) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P4 (SEQ ID NO:370) and PLO1_HUMAN_V1 (SEQ ID NO:368):

1. An isolated chimeric polypeptide encoding for HUMLYSYL_PEA_{—}1_P4 (SEQ ID NO:370), comprising a first amino acid sequence being at least 90% homologous to MRPLLLLALLGWLLLAEAKGDAKPE corresponding to amino acids 1-25 of PLO1_HUMAN_V1 (SEQ ID NO:368), which also corresponds to amino acids 1-25 of HUMLYSYL_PEA_{—}1_P4 (SEQ ID NO:370), a second amino acid sequence being at least 70%, optionally at least 80%, preferably at least 85%, more preferably at least 90% and most preferably at least 95% homologous to a polypeptide having the sequence APCCQEGLRAGGSGSLHLGRDFTVLAGARGSPSPSVSSIPRFWIPGS (SEQ ID NO:504) corresponding to amino acids 26-72 of HUMLYSYL_PEA_{—}1_P4 (SEQ ID NO:370), and a third amino acid sequence being at least 90% homologous to DNLLVLTVATKETEGFRRFKRSAQFFNYKIQALGLGEDWNVEKGTSAGGGQKVRLLK KALEKHADKEDLVILFADSYDVLFASGPRELLKKFRQARSQVVFSAEELIYPDRRLETK YPVVSDGKRFLGSGGFIGYAPNLSKLVAEWEGQDSDSDQLFYTKIFLDPEKREQINITLD HRCRIFQNLDGALDEVVLKFEMGHVRARNLAYDTLPVLIHGNGPTKLQLNYLGNYIPR FWTFETGCTVCDEGLRSLKGIGDEALPTVLVGVFIEQPTPFVSLFFQRLLRLHYPQKHMR LFIHNHEQHHKAQVEEFLAQHGSEYQSVKLVGPEVRMANADARNMGADLCRQDRSCT YYFSVDADVALTEPNSLRLLIQQNKNVIAPLMTRHGRLWSNFWGALSADGYYARSED YVDIVQGRRVGVWNVPYISNIYLIKGSALRGELQS SDLFHHSKLDPDMAFCANIRQQDV FMFLTNRHTLGHLLSLDSYRTTHLHNDLWEVFSNPEDWKEKYIHQNYTKALAGKLVET PCPDVYWFPIFTEVACDELVEEMEHFGQWSLGNNKDNRIQGGYENVPTIDIHMNQIGFE REWHKFLLEYIAPMTEKLYPGYYTRAQFDLAFVVRYKPDEQPSLMPHHDASTFTINIAL NRVGVDYEGGGCRFLRYNCSIRAPRKGWTLMHPGRLTHYHEGLPTTRGTRYIAVSFVD P corresponding to amino acids 26-727 of PLO1_HUMAN_V1 (SEQ ID NO:368), which also corresponds to amino acids 73-774 of HUMLYSYL_PEA_{—}1_P4 (SEQ ID NO:370), wherein said first amino acid sequence, second amino acid sequence and third amino acid sequence are contiguous and in a sequential order.

2. An isolated polypeptide encoding for an edge portion of HUMLYSYL_PEA_{—}1_P4 (SEQ ID NO:370), comprising an amino acid sequence being at least 70%, optionally at least about 80%, preferably at least about 85%, more preferably at least about 90% and most preferably at least about 95% homologous to the sequence encoding for APCCQEGLRAGGSGSLHLGRDFTVLAGARGSPSPSVSSIPRFWIPGS (SEQ ID NO:504), corresponding to HUMLYSYL_PEA_{—}1_P4 (SEQ ID NO:370).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 8
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P4 (SEQ ID NO:370) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HUMLYSYL_PEA_{—}1_P4 (SEQ ID NO:370) Sequence provides support for the deduced sequence of this variant protein according to the present invention).

TABLE 9
Amino acid mutations

| SNP position(s) | | |
|---|---|---|
| on amino acid | Alternative | Previously |
| sequence | amino acid(s) | known SNP? |
| 114 | E -> D | Yes |
| 145 | F -> | No |
| 146 | A -> T | Yes |
| 167 | A -> S | Yes |
| 225 | S -> | No |
| 226 | D -> N | No |
| 251 | C -> | No |
| 279 | A -> | No |
| 279 | A -> G | No |
| 357 | R -> W | Yes |
| 428 | V -> M | Yes |
| 433 | A -> | No |
| 681 | R -> | No |
| 693 | K -> N | Yes |
| 701 | M -> I | Yes |
| 762 | R -> | No |
| 764 | T -> | No |

Variant protein HUMLYSYL_PEA_{—}1_P4 (SEQ ID NO:370) is encoded by the following transcript(s): HUMLYSYL_PEA_{—}1_T4 (SEQ ID NO:314), for which the sequence(s) is/are given at the end of the application. The coding portion of transcript HUMLYSYL_PEA_{—}1_T4 (SEQ ID NO:314) is shown in bold; this coding portion starts at position 104 and ends at position 2425. The transcript also has the following SNPs as listed in Table 10 (given according to their position on the nucleotide sequence, with the alternative nucleic acid listed; the last column indicates whether the SNP is known or not; the presence of known SNPs in variant protein HUMLYSYL_PEA_{—}1_P4 (SEQ ID NO:370) Sequence provides support for the deduced sequence of this variant protein according to the present invention).

TABLE 10
Nucleic acid SNPs

| SNP position | | |
|---|---|---|
| on nucleotide | Alternative | Previously |
| sequence | nucleic acid | known SNP? |
| 37 | C -> | No |
| 71 | C -> | No |
| 102 | C -> | No |
| 358 | C -> A | Yes |
| 445 | G -> C | Yes |
| 511 | A -> G | Yes |
| 538 | C -> | No |
| 538 | C -> T | Yes |
| 539 | G -> A | Yes |
| 602 | G -> T | Yes |
| 777 | G -> | No |
| 779 | G -> A | No |
| 856 | C -> | No |
| 939 | C -> | No |
| 939 | C -> G | No |
| 1172 | C -> T | Yes |
| 1385 | G -> A | Yes |
| 1401 | C -> | No |
| 1450 | C -> T | Yes |
| 1630 | G -> C | No |
| 1876 | A -> C | Yes |
| 2145 | G -> | No |
| 2176 | C -> T | Yes |
| 2182 | G -> C | Yes |
| 2206 | G -> T | Yes |
| 2368 | T -> C | Yes |
| 2377 | C -> G | Yes |
| 2388 | G -> | No |
| 2394 | C -> | No |
| 2492 | G -> | No |
| 2499 | C -> G | Yes |
| 2505 | C -> | No |
| 2629 | C -> | No |
| 2629 | C -> T | No |
| 2649 | C -> | No |
| 2686 | G -> A | No |
| 2725 | C -> | No |
| 2725 | C -> G | No |
| 2739 | C -> T | No |
| 2812 | G -> A | No |
| 2812 | G -> C | No |
| 2852 | G -> | No |
| 2859 | C -> T | Yes |
| 2868 | G -> C | Yes |
| 2961 | C -> | No |
| 2975 | G -> C | Yes |
| 3027 | C -> T | Yes |
| 3042 | G -> T | Yes |
| 3098 | C -> A | Yes |

Variant protein HUMLYSYL_PEA_{—}1_P5 (SEQ ID NO:371) according to the present invention has an amino acid sequence as given at the end of the application; it is encoded by transcript(s) HUMLYSYL_PEA_{—}1_T5 (SEQ ID NO:315). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P5 (SEQ ID NO:371) and PLO1_HUMAN_V1 (SEQ ID NO:368):

1. An isolated chimeric polypeptide encoding for HUMLYSYL_PEA_{—}1_P5 (SEQ ID NO:371), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIG corresponding to amino acids 1-281 of PLO1_HUMAN_V1 (SEQ ID NO:368), which also corresponds to amino acids 1-281 of HUMLYSYL_PEA_{—}1_P5 (SEQ ID NO:371), and a second amino acid sequence being at least 90% homologous to RLLRLHYPQKHMRLFIHNHEQHHKAQVEEFLAQHGSEYQSVKLVGPEVRMANADARN MGADLCRQDRSCTYYFSVDADVALTEPNSLRLLIQQNKNVIAPLMTRHGRLWSNFWG ALSADGYYARSEDYVDIVQGRRVGVWNVPYISNIYLIKGSALRGELQSSDLFHHSKLDP DMAFCANIRQQDVFMFLTNRHTLGHLLSLDSYRTTHLHNDLWEVFSNPEDWKEKYIH QNYTKALAGKLVETPCPDVYWFPIFTEVACDELVEEMEHFGQWSLGNNKDNRIQGGY ENVPTIDIHMNQIGFEREWHKFLLEYIAPMTEKLYPGYYTRAQFDLAFVVRYKPDEQPS LMPHHDASTFTINIALNRVGVDYEGGGCRFLRYNCSIRAPRKGWTLMHPGRLTHYHEG LPTTRGTRYIAVSFVDP corresponding to amino acids 307-727 of PLO1_HUMAN_V1 (SEQ ID NO:368), which also corresponds to amino acids 282-702 of HUMLYSYL_PEA_{—}1_P5 (SEQ ID NO:371), wherein said first amino acid sequence and second amino acid sequence are contiguous and in a sequential order.

2. An isolated chimeric polypeptide encoding for an edge portion of HUMLYSYL_PEA_{—}1_P5 (SEQ ID NO:371), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GR, having a structure as follows: a sequence starting from any of amino acid numbers 281-x to 281; and ending at any of amino acid numbers 282+((n−2)−x), in which x varies from 0 to n−2.

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 11
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P5 (SEQ ID NO:371) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein HUMLYSYL_PEA_{—}1_P5 (SEQ ID NO:371) Sequence provides support for the deduced sequence of this variant protein according to the present invention).

TABLE 12
Amino acid mutations

| SNP position(s) | | |
|---|---|---|
| on amino acid | Alternative | Previously |
| sequence | amino acid(s) | known SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 232 | A -> G | No |
| 232 | A -> | No |
| 285 | R -> W | Yes |
| 356 | V -> M | Yes |
| 361 | A -> | No |
| 609 | R -> | No |
| 621 | K -> N | Yes |
| 629 | M -> I | Yes |
| 690 | R -> | No |
| 692 | T -> | No |

Variant protein HUMLYSYL_PEA_{—}1_P5 (SEQ ID NO:371) is encoded by the following transcript(s): HUMLYSYL_PEA_{—}1_T5 (SEQ ID NO:315), for which the sequence(s) is/are given at the end of the application. The coding portion of transcript HUMLYSYL_PEA_T5 (SEQ ID NO:315) is shown in bold; this coding portion starts at position 104 and ends at position 2209. The transcript also has the following SNPs as listed in Table 13 (given according to their position on the nucleotide sequence, with the alternative nucleic acid listed; the last column indicates whether the SNP is known or not; the presence of known SNPs in variant protein HUMLYSYL_PEA_{—}1_P5 (SEQ ID NO:371) Sequence provides support for the deduced sequence of this variant protein according to the present invention).

TABLE 13
Nucleic acid SNPs

| SNP position on | Alternative | Previously known |
|---|---|---|
| nucleotide sequence | nucleic acid | SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 798 | C -> | No |
| 798 | C -> G | No |
| 956 | C -> T | Yes |
| 1169 | G -> A | Yes |
| 1185 | C -> | No |
| 1234 | C -> T | Yes |
| 1414 | G -> C | No |
| 1660 | A -> C | Yes |
| 1929 | G -> | No |
| 1960 | C -> T | Yes |
| 1966 | G -> C | Yes |
| 1990 | G -> T | Yes |
| 2152 | T -> C | Yes |
| 2161 | C -> G | Yes |
| 2172 | G -> | No |
| 2178 | C -> | No |
| 2276 | G -> | No |
| 2283 | C -> G | Yes |
| 2289 | C -> | No |
| 2413 | C -> | No |
| 2413 | C -> T | No |
| 2433 | C -> | No |
| 2470 | G -> A | No |
| 2509 | C -> | No |
| 2509 | C -> G | No |
| 2523 | C -> T | No |
| 2596 | G -> A | No |
| 2596 | G -> C | No |
| 2636 | G -> | No |
| 2643 | C -> T | Yes |
| 2652 | G -> C | Yes |
| 2745 | C -> | No |
| 2759 | G -> C | Yes |
| 2811 | C -> T | Yes |
| 2826 | G -> T | Yes |
| 2882 | C -> A | Yes |

Variant protein HUMLYSYL_PEA_{—}1_P6 (SEQ ID NO:372) according to the present invention has an amino acid sequence as given at the end of the application; it is encoded by transcript(s) HUMLYSYL_PEA_{—}1_T6 (SEQ ID NO:316). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P6 (SEQ ID NO:372) and PLO1_HUMAN_V1 (SEQ ID NO:368):

1. An isolated chimeric polypeptide encoding for HUMLYSYL_PEA_{—}1_P6 (SEQ ID NO:372), comprising a first amino acid sequence being at least 90% homologous to MRPLLLLALLGWLLLAEAKGDAKPEDNLLVLTVATKETEGFRRFKRSAQFFNYKI corresponding to amino acids 1-55 of PLO1_HUMAN_V1 (SEQ ID NO:368), which also corresponds to amino acids 1-55 of HUMLYSYL_PEA_{—}1_P6 (SEQ ID NO:372), a second amino acid sequence being at least 70%, optionally at least 80%, preferably at least 85%, more preferably at least 90% and most preferably at least 95% homologous to a polypeptide having the sequence QPVLRGVSL (SEQ ID NO:505) corresponding to amino acids 56-64 of HUMLYSYL_PEA_{—}1_P6 (SEQ ID NO:372), and a third amino acid sequence being at least 90% homologous to QALGLGEDWNVEKGTSAGGGQKVRLLKKALEKHADKEDLVILFADSYDVLFASGPRE LLKKFRQARSQVVFSAEELIYPDRRLETKYPVVSDGKRFLGSGGFIGYAPNLSKLVAEW EGQDSDSDQLFYTKIFLDPEKREQINITLDHRCRIFQNLDGALDEVVLKFEMGHVRARN LAYDTLPVLIHGNGPTKLQLNYLGNYIPRFWTFETGCTVCDEGLRSLKGIGDEALPTVL VGVFIEQPTPFVSLFFQRLLRLHYPQKHMRLFIHNHEQHHKAQVEEFLAQHGSEYQSVK LVGPEVRMANADARNMGADLCRQDRSCTYYFSVDADVALTEPNSLRLLIQQNKNVIA PLMTRHGRLWSNFWGALSADGYYARSEDYVDIVQGRRVGVWNVPYISNIYLIKGSALR GELQSSDLFHHSKLDPDMAFCANIRQQDVFMFLTNRHTLGHLLSLDSYRTTHLHNDLW EVFSNPEDWKEKYIHQNYTKALAGKLVETPCPDVYWFPIFTEVACDELVEEMEHFGQW SLGNNKDNRIQGGYENVPTIDIHMNQIGFEREWHKFLLEYIAPMTEKLYPGYYTRAQFD LAFVVRYKPDEQPSLMPHHDASTFTINIALNRVGVDYEGGGCRFLRYNCSIRAPRKGW TLMHPGRLTHYHEGLPTTRGTRYIAVSFVDP corresponding to amino acids 56-727 of PLO1_HUMAN_V1 (SEQ ID NO:368), which also corresponds to amino acids 65-736 of HUMLYSYL_PEA_{—}1_P6 (SEQ ID NO:372), wherein said first amino acid sequence, second amino acid sequence and third amino acid sequence are contiguous and in a sequential order.

2. An isolated polypeptide encoding for an edge portion of HUMLYSYL_PEA_{—}1_P6 (SEQ ID NO:372), comprising an amino acid sequence being at least 70%, optionally at least about 80%, preferably at least about 85%, more preferably at least about 90% and most preferably at least about 95% homologous to the sequence encoding for QPVLRGVSL (SEQ ID NO:505), corresponding to HUMLYSYL_PEA_{—}1_P6 (SEQ ID NO:372).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 14
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P6 (SEQ ID NO:372) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein HUMLYSYL_PEA_{—}1_P6 (SEQ ID NO:372) Sequence provides support for the deduced sequence of this variant protein according to the present invention).

TABLE 15
Amino acid mutations

| SNP position(s) on | Alternative | Previously known |
|---|---|---|
| amino acid sequence | amino acid(s) | SNP? |
| 76 | E -> D | Yes |
| 107 | F -> | No |
| 108 | A -> T | Yes |
| 129 | A -> S | Yes |
| 187 | S -> | No |
| 188 | D -> N | No |
| 213 | C -> | No |
| 241 | A -> | No |
| 241 | A -> G | No |
| 319 | R -> W | Yes |
| 390 | V -> M | Yes |
| 395 | A -> | No |
| 643 | R -> | No |
| 655 | K -> N | Yes |
| 663 | M -> I | Yes |
| 724 | R -> | No |
| 726 | T -> | No |

Variant protein HUMLYSYL_PEA_{—}1_P6 (SEQ ID NO:372) is encoded by the following transcript(s): HUMLYSYL_PEA_{—}1_T6 (SEQ ID NO:316), for which the sequence(s) is/are given at the end of the application. The coding portion of transcript HUMLYSYL_PEA_{—}1_T6 (SEQ ID NO:316) is shown in bold; this coding portion starts at position 104 and ends at position 2311. The transcript also has the following SNPs as listed in Table 16 (given according to their position on the nucleotide sequence, with the alternative nucleic acid listed; the last column indicates whether the SNP is known or not; the presence of known SNPs in variant protein HUMLYSYL_PEA_{—}1_P6 (SEQ ID NO:372) Sequence provides support for the deduced sequence of this variant protein according to the present invention).

TABLE 16
Nucleic acid SNPs

| SNP position on | Alternative | Previously known |
|---|---|---|
| nucleotide sequence | nucleic acid | SNP? |
| 37 | C -> | No |
| 71 | C -> | No |
| 102 | C -> | No |
| 217 | C -> A | Yes |
| 331 | G -> C | Yes |
| 397 | A -> G | Yes |
| 424 | C -> | No |
| 424 | C -> T | Yes |
| 425 | G -> A | Yes |
| 488 | G -> T | Yes |
| 663 | G -> | No |
| 665 | G -> A | No |
| 742 | C -> | No |
| 825 | C -> | No |
| 825 | C -> G | No |
| 1058 | C -> T | Yes |
| 1271 | G -> A | Yes |
| 1287 | C -> | No |
| 1336 | C -> T | Yes |
| 1516 | G -> C | No |
| 1762 | A -> C | Yes |
| 2031 | G -> | No |
| 2062 | C -> T | Yes |
| 2068 | G -> C | Yes |
| 2092 | G -> T | Yes |
| 2254 | T -> C | Yes |
| 2263 | C -> G | Yes |
| 2274 | G -> | No |
| 2280 | C -> | No |
| 2378 | G -> | No |
| 2385 | C -> G | Yes |
| 2391 | C -> | No |
| 2515 | C -> | No |
| 2515 | C -> T | No |
| 2535 | C -> | No |
| 2572 | G -> A | No |
| 2611 | C -> | No |
| 2611 | C -> G | No |
| 2625 | C -> T | No |
| 2698 | G -> A | No |
| 2698 | G -> C | No |
| 2738 | G -> | No |
| 2745 | C -> T | Yes |
| 2754 | G -> C | Yes |
| 2847 | C -> | No |
| 2861 | G -> C | Yes |
| 2913 | C -> T | Yes |
| 2928 | G -> T | Yes |
| 2984 | C -> A | Yes |

Variant protein HUMLYSYL_PEA_{—}1_P7 (SEQ ID NO:373) according to the present invention has an amino acid sequence as given at the end of the application; it is encoded by transcript(s) HUMLYSYL_PEA_{—}1_T9 (SEQ ID NO:318). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P7 (SEQ ID NO:373) and PLO1_HUMAN_V1 (SEQ ID NO:368):

1. An isolated chimeric polypeptide encoding for HUMLYSYL_PEA_{—}1_P7 (SEQ ID NO:373), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGAL corresponding to amino acids 1-214 of PLO1_HUMAN_V1 (SEQ ID NO:368), which also corresponds to amino acids 1-214 of HUMLYSYL_PEA_{—}1_P7 (SEQ ID NO:373), a second amino acid sequence being at least 70%, optionally at least 80%, preferably at least 85%, more preferably at least 90% and most preferably at least 95% homologous to a polypeptide having the sequence VSPWGQGHLPGACYELTASVLTSELSVMPSFPA (SEQ ID NO:506) corresponding to amino acids 215-247 of HUMLYSYL_PEA_{—}1_P7 (SEQ ID NO:373), a third amino acid sequence being at least 90% homologous to VV corresponding to amino acids 217-218 of PLO1_HUMAN_V1 (SEQ ID NO:368), which also corresponds to amino acids 248-249 of HUMLYSYL_PEA_{—}1_P7 (SEQ ID NO:373), and a fourth amino acid sequence being at least 90% homologous to LQLNYLGNYIPRFWTFETGCTVCDEGLRSLKGIGDEALPTVLVGVFIEQPTPFVSLFFQR LLRLHYPQKHMRLFIHNHEQHHKAQVEEFLAQHGSEYQSVKLVGPEVRMANADARN MGADLCRQDRSCTYYFSVDADVALTEPNSLRLLIQQNKNVIAPLMTRHGRLWSNFWG ALSADGYYARSEDYVDIVQGRRVGVWNVPYISNIYLIKGSALRGELQSSDLFHHSKLDP DMAFCANIRQQDVFMFLTNRHTLGHLLSLDSYRTTHLHNDLWEVFSNPEDWKEKYIH QNYTKALAGKLVETPCPDVYWFPIFTEVACDELVEEMEHFGQWSLGNNKDNRIQGGY ENVPTIDIHMNQIGFEREWHKFLLEYIAPMTEKLYPGYYTRAQFDLAFVVRYKPDEQPS LMPHHDASTFTINIALNRVGVDYEGGGCRFLRYNCSIRAPRKGWTLMHPGRLTHYHEG LPTTRGTRYIAVSFVDP corresponding to amino acids 248-727 of PLO1_HUMAN_V1 (SEQ ID NO:368), which also corresponds to amino acids 250-729 of HUMLYSYL_PEA_{—}1_P7 (SEQ ID NO:373), wherein said first amino acid sequence, second amino acid sequence, third amino acid sequence and fourth amino acid sequence are contiguous and in a sequential order.

2. An isolated polypeptide encoding for an edge portion of HUMLYSYL_PEA_{—}1_P7 (SEQ ID NO:373), comprising an amino acid sequence being at least 70%, optionally at least about 80%, preferably at least about 85%, more preferably at least about 90% and most preferably at least about 95% homologous to the sequence encoding for VSPWGQGHLPGACYELTASVLTSELSVMPSFPA (SEQ ID NO:506), corresponding to HUMLYSYL_PEA_{—}1_P7 (SEQ ID NO:373).

3. A bridge portion of HUMLYSYL_PEA_{—}1_P7 (SEQ ID NO:373), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise LV, having a structure as follows (numbering according to HUMLYSYL_PEA_{—}1_P7 (SEQ ID NO:373)): a sequence starting from any of amino acid numbers 214−x to 214; and ending at any of amino acid numbers 215+((n−2)−x), in which x varies from 0 to n−2.

4. An isolated chimeric polypeptide encoding for an edge portion of HUMLYSYL_PEA_{—}1_P7 (SEQ ID NO:373), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VL, having a structure as follows: a sequence starting from any of amino acid numbers 249−x to 249; and ending at any of amino acid numbers 250+((n−2)−x), in which x varies from 0 to n−2.

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 17
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P7 (SEQ ID NO:373) also has the following non-silent SNPs (Single Nucleotide Polymorphisms) as listed in Table 18, (given according to their position(s) on the amino acid sequence, with the alternative amino acid(s) listed; the last column indicates whether the SNP is known or not; the presence of known SNPs in variant protein HUMLYSYL_PEA_{—}1_P7 (SEQ ID NO:373) Sequence provides support for the deduced sequence of this variant protein according to the present invention).

TABLE 18
Amino acid mutations

| SNP position(s) on | Alternative | Previously known |
|---|---|---|
| amino acid sequence | amino acid(s) | SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 312 | R -> W | Yes |
| 383 | V -> M | Yes |
| 388 | A -> | No |
| 636 | R -> | No |
| 648 | K -> N | Yes |
| 656 | M -> I | Yes |
| 717 | R -> | No |
| 719 | T -> | No |

Variant protein HUMLYSYL_PEA_{—}1_P7 (SEQ ID NO:373) is encoded by the following transcript(s): HUMLYSYL_PEA_{—}1_T9 (SEQ ID NO:318), for which the sequence(s) is/are given at the end of the application. The coding portion of transcript HUMLYSYL_PEA_{—}1_T9 (SEQ ID NO:318) is shown in bold; this coding portion starts at position 104 and ends at position 2290. The transcript also has the following SNPs as listed in Table 19 (given according to their position on the nucleotide sequence, with the alternative nucleic acid listed; the last column indicates whether the SNP is known or not; the presence of known SNPs in variant protein HUMLYSYL_PEA_{—}1_P7 (SEQ ID NO:373) Sequence provides support for the deduced sequence of this variant protein according to the present invention).

TABLE 19
Nucleic acid SNPs

| SNP position on | Alternative | Previously known |
|---|---|---|
| nucleotide sequence | nucleic acid | SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 1037 | C -> T | Yes |
| 1250 | G -> A | Yes |
| 1266 | C -> | No |
| 1315 | C -> T | Yes |
| 1495 | G -> C | No |
| 1741 | A -> C | Yes |
| 2010 | G -> | No |
| 2041 | C -> T | Yes |
| 2047 | G -> C | Yes |
| 2071 | G -> T | Yes |
| 2233 | T -> C | Yes |
| 2242 | C -> G | Yes |
| 2253 | G -> | No |
| 2259 | C -> | No |
| 2357 | G -> | No |
| 2364 | C -> G | Yes |
| 2370 | C -> | No |
| 2494 | C -> | No |
| 2494 | C -> T | No |
| 2514 | C -> | No |
| 2551 | G -> A | No |
| 2590 | C -> | No |
| 2590 | C -> G | No |
| 2604 | C -> T | No |
| 2677 | G -> A | No |
| 2677 | G -> C | No |
| 2717 | G -> | No |
| 2724 | C -> T | Yes |
| 2733 | G -> C | Yes |
| 2826 | C -> | No |
| 2840 | G -> C | Yes |
| 2892 | C -> T | Yes |
| 2907 | G -> T | Yes |
| 2963 | C -> A | Yes |

Variant protein HUMLYSYL_PEA_{—}1_P13 (SEQ ID NO:374) according to the present invention has an amino acid sequence as given at the end of the application; it is encoded by transcript(s) HUMLYSYL PEA_{—}1_T19 (SEQ ID NO:319). An alignment is given to the known protein (Procollagen-lysine,2-oxoglutarate 5-dioxygenase 1 precursor (SEQ ID NO:367) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P13 (SEQ ID NO:374) and PLO1_HUMAN_V1 (SEQ ID NO:368):

An isolated chimeric polypeptide encoding for HUMLYSYL_PEA_{—}1_P13 (SEQ ID NO:374), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVETPCPDVYWFPIFTEVACDELVEEMEHFGQWSLG NNK corresponding to amino acids 1-585 of PLO1_HUMAN_V1 (SEQ ID NO:368), which also corresponds to amino acids 1-585 of HUMLYSYL_PEA_{—}1_P13 (SEQ ID NO:374), and a second amino acid sequence being at least 70%, optionally at least 80%, preferably at least 85%, more preferably at least 90% and most preferably at least 95% homologous to a polypeptide having the sequence GCPESGTSASMAGHESKP (SEQ ID NO:475) corresponding to amino acids 586-603 of HUMLYSYL_PEA_{—}1_P13 (SEQ ID NO:374), wherein said first amino acid sequence and second amino acid sequence are contiguous and in a sequential order.

2. An isolated polypeptide encoding for a tail of HUMLYSYL_PEA_{—}1_P13 (SEQ ID NO:3741, comprising a polypeptide being at least 70%, optionally at least about 80%, preferably at least about 85%, more preferably at least about 90% and most preferably at least about 95% homologous to the sequence GCPESGTSASMAGHESKP (SEQ ID NO:475) in HUMLYSYL_PEA_{—}1_P13 (SEQ ID NO:374).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 20
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P13 (SEQ ID NO:374) also has the following non-silent SNPs (Single Nucleotide Polymorphisms) as listed in Table 21, (given according to their position(s) on the amino acid sequence, with the alternative amino acid(s) listed; the last column indicates whether the SNP is known or not; the presence of known SNPs in variant protein HUMLYSYL_PEA_{—}1_P13 (SEQ ID NO:374) Sequence provides support for the deduced sequence of this variant protein according to the present invention).

TABLE 21
Amino acid mutations

| SNP position(s) on | Alternative | Previously known |
|---|---|---|
| amino acid sequence | amino acid(s) | SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 232 | A -> G | No |
| 232 | A -> | No |
| 310 | R -> W | Yes |
| 381 | V -> M | Yes |
| 386 | A -> | No |

Variant protein HUMLYSYL_PEA_{—}1_P13 (SEQ ID NO:374) is encoded by the following transcript(s): HUMLYSYL_PEA_{—}1_T19 (SEQ ID NO:319), for which the sequence(s) is/are given at the end of the application. The coding portion of transcript HUMLYSYL_PEA_{—}1_T19 (SEQ ID NO:319) is shown in bold; this coding portion starts at position 104 and ends at position 1912. The transcript also has the following SNPs as listed in Table 22 (given according to their position on the nucleotide sequence, with the alternative nucleic acid listed; the last column indicates whether the SNP is known or not; the presence of known SNPs in variant protein HUMLYSYL_PEA_{—}1_P13 (SEQ ID NO:374) Sequence provides support for the deduced sequence of this variant protein according to the present invention).

TABLE 22
Nucleic acid SNPs

| SNP position on | Alternative | Previously known |
|---|---|---|
| nucleotide sequence | nucleic acid | SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 798 | C -> | No |
| 798 | C -> G | No |
| 1031 | C -> T | Yes |
| 1244 | G -> A | Yes |
| 1260 | C -> | No |
| 1309 | C -> T | Yes |
| 1489 | G -> C | No |
| 1735 | A -> C | Yes |
| 1917 | C -> | No |
| 1931 | G -> C | Yes |
| 1983 | C -> T | Yes |
| 1998 | G -> T | Yes |
| 2054 | C -> A | Yes |

Variant protein HUMLYSYL_PEA_{—}1_P14 (SEQ ID NO:375) according to the present invention has an amino acid sequence as given at the end of the application; it is encoded by transcript(s) HUMLYSYL_PEA_{—}1_T20 (SEQ ID NO:320). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P14 (SEQ ID NO:375) and PLO1_HUMAN_V1 (SEQ ID NO:368):

1. An isolated chimeric polypeptide encoding for HUMLYSYL_PEA_{—}1_P14 (SEQ ID NO:375), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVETPCPDVYWFPIFTEVACDELVEEMEHFGQWSLG NNK corresponding to amino acids 1-585 of PLO1_HUMAN_V1 (SEQ ID NO:368), which also corresponds to amino acids 1-585 of HUMLYSYL_PEA_{—}1_P14 (SEQ ID NO:375), and a second amino acid sequence being at least 70%, optionally at least 80%, preferably at least 85%, more preferably at least 90% and most preferably at least 95% homologous to a polypeptide having the sequence TATPENLLGDRRGICAQLDLLLACGEGSDRSTHHTGSPCPGCL (SEQ ID NO:476) corresponding to amino acids 586-628 of HUMLYSYL_PEA_{—}1_P14 (SEQ ID NO:375), wherein said first amino acid sequence and second amino acid sequence are contiguous and in a sequential order.

2. An isolated polypeptide encoding for a tail of HUMLYSYL_PEA_{—}1_P14 (SEQ ID NO:375), comprising a polypeptide being at least 70%, optionally at least about 80%, preferably at least about 85%, more preferably at least about 90% and most preferably at least about 95% homologous to the sequence TATPENLLGDRRGICAQLDLLLACGEGSDRSTHHTGSPCPGCL (SEQ ID NO:476) in HUMLYSYL_PEA_{—}1_P14 (SEQ ID NO:375).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 23
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P14 (SEQ ID NO:375) also has the following non-silent SNPs (Single Nucleotide Polymorphisms) as listed in Table 24, (given according to their position(s) on the amino acid sequence, with the alternative amino acid(s) listed; the last column indicates whether the SNP is known or not; the presence of known SNPs in variant protein HUMLYSYL_PEA_{—}1_P14 (SEQ ID NO:375) Sequence provides support for the deduced sequence of this variant protein according to the present invention).

TABLE 24
Amino acid mutations

| SNP position(s) on amino acid | Alternative amino | Previously known |
|---|---|---|
| sequence | acid(s) | SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 232 | A -> G | No |
| 232 | A -> | No |
| 310 | R -> W | Yes |
| 381 | V -> M | Yes |
| 386 | A -> | No |
| 605 | L -> F | Yes |
| 610 | G -> W | Yes |

Variant protein HUMLYSYL_PEA_{—}1_P14 (SEQ ID NO:375) is encoded by the following transcript(s): HUMLYSYL_PEA_{—}1_T20 (SEQ ID NO:320), for which the sequence(s) is/are given at the end of the application. The coding portion of transcript HUMLYSYL_PEA_{—}1_T20 (SEQ ID NO:320) is shown in bold; this coding portion starts at position 104 and ends at position 1987. The transcript also has the following SNPs as listed in Table 25 (given according to their position on the nucleotide sequence, with the alternative nucleic acid listed; the last column indicates whether the SNP is known or not; the presence of known SNPs in variant protein HUMLYSYL_PEA_{—}1_P14 (SEQ ID NO:375) Sequence provides support for the deduced sequence of this variant protein according to the present invention).

TABLE 25
Nucleic acid SNPs

| SNP position on nucleotide | Alternative nucleic | Previously known |
|---|---|---|
| sequence | acid | SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 798 | C -> | No |
| 798 | C -> G | No |
| 1031 | C -> T | Yes |
| 1244 | G -> A | Yes |
| 1260 | C -> | No |
| 1309 | C -> T | Yes |
| 1489 | G -> C | No |
| 1735 | A -> C | Yes |
| 1864 | G -> C | Yes |
| 1916 | C -> T | Yes |
| 1931 | G -> T | Yes |
| 1987 | C -> A | Yes |

Variant protein HUMLYSYL_PEA_{—}1_P16 (SEQ ID NO:376) according to the present invention has an amino acid sequence as given at the end of the application; it is encoded by transcript(s) HUMLYSYL_PEA_{—}1_T22 (SEQ ID NO:321). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P16 (SEQ ID NO:376) and PLO1_HUMAN_V1 (SEQ ID NO:368):

1. An isolated chimeric polypeptide encoding for HUMLYSYL_PEA_{—}1_P16 (SEQ ID NO:376), comprising a first amino acid sequence being at least 90% homologous to MRPLLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKREQINITLDHRCRIFQNLDGALDEVVLKFEMGHVRARNLAY DTLPVLIHGNGPTKLQLNYLGNYIPRFWTFETGCTVCDEGLRSLKGIGDEALPTVLVGV FIEQPTPFVSLFFQRLLRLHYPQKHMRLFIHNHEQHHKAQVEEFLAQHGSEYQSVKLVG PEVRMANADARNMGADLCRQDRSCTYYFSVDADVALTEPNSLRLLIQQNKNVIAPLM TRHGRLWSNFWGALSADGYYARSEDYVDIVQGRRVGVWNVPYISNIYLIKGSALRGEL QSSDLFHHSKLDPDMAFCANIRQQDVFMFLTNRHTLGHLLSLDSYRTTHLHNDLWEVF SNPEDWKEKYIHQNYTKALAGKLVET corresponding to amino acids 1-550 of PLO1_HUMAN_V1 (SEQ ID NO:368), which also corresponds to amino acids 1-550 of HUMLYSYL_PEA_{—}1_P16 (SEQ ID NO:376), and a second amino acid sequence being at least 70%, optionally at least 80%, preferably at least 85%, more preferably at least 90% and most preferably at least 95% homologous to a polypeptide having the sequence VRAMDTLLDQPCLLQGAGHRRETACPGEWGTAGWEL (SEQ ID NO:477) corresponding to amino acids 551-586 of HUMLYSYL_PEA_{—}1_P16 (SEQ ID NO:376), wherein said first amino acid sequence and second amino acid sequence are contiguous and in a sequential order.

2. An isolated polypeptide encoding for a tail of HUMLYSYL_PEA_{—}1_P16 (SEQ ID NO:376), comprising a polypeptide being at least 70%, optionally at least about 80%, preferably at least about 85%, more preferably at least about 90% and most preferably at least about 95% homologous to the sequence VRAMDTLLDQPCLLQGAGHRRETACPGEWGTAGWEL (SEQ ID NO:477) in HUMLYSYL_PEA_{—}1_P16 (SEQ ID NO:376).

It should be noted that the known protein sequence (PLO1_HUMAN) Has one or more changes than the sequence given at the end of the application and named as being the amino acid sequence for PLO1_HUMAN_V1 (SEQ ID NO:368). These changes were previously known to occur and are listed in the table below.

TABLE 26
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P16 (SEQ ID NO:376) also has the following non-silent SNPs (Single Nucleotide Polymorphisms) as listed in Table 27, (given according to their position(s) on the amino acid sequence, with the alternative amino acid(s) listed; the last column indicates whether the SNP is known or not; the presence of known SNPs in variant protein HUMLYSYL_PEA_{—}1_P16 (SEQ ID NO:376) Sequence provides support for the deduced sequence of this variant protein according to the present invention).

TABLE 27
Amino acid mutations

| SNP position(s) on amino acid | Alternative amino | Previously known |
|---|---|---|
| sequence | acid(s) | SNP? |
| 67 | E -> D | Yes |
| 98 | F -> | No |
| 99 | A -> T | Yes |
| 120 | A -> S | Yes |
| 178 | S -> | No |
| 179 | D -> N | No |
| 204 | C -> | No |
| 232 | A -> G | No |
| 232 | A -> | No |
| 310 | R -> W | Yes |
| 381 | V -> M | Yes |
| 386 | A -> | No |

Variant protein HUMLYSYL_PEA_{—}1_P16 (SEQ ID NO:376) is encoded by the following transcript(s): HUMLYSYL_PEA_{—}1_T22 (SEQ ID NO:321), for which the sequence(s) is/are given at the end of the application. The coding portion of transcript HUMLYSYL_PEA_{—}1_T22 (SEQ ID NO:321) is shown in bold; this coding portion starts at position 104 and ends at position 88889. The transcript also has the following SNPs as listed in Table 28 (given according to their position on the nucleotide sequence, with the alternative nucleic acid listed; the last column indicates whether the SNP is known or not; the presence of known SNPs in variant protein HUMLYSYL_PEA_{—}1_P16 (SEQ ID NO:376) Sequence provides support for the deduced sequence of this variant protein according to the present invention).

TABLE 28
Nucleic acid SNPs

| SNP position on nucleotide | Alternative nucleic | Previously known |
|---|---|---|
| sequence | acid | SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 715 | C -> | No |
| 798 | C -> | No |
| 798 | C -> G | No |
| 1031 | C -> T | Yes |
| 1244 | G -> A | Yes |
| 1260 | C -> | No |
| 1309 | C -> T | Yes |
| 1489 | G -> C | No |
| 1735 | A -> C | Yes |

Variant protein HUMLYSYL_PEA_{—}1_P18 (SEQ ID NO:377) according to the present invention has an amino acid sequence as given at the end of the application; it is encoded by transcript(s) HUMLYSYL_PEA_{—}1_T24 (SEQ ID NO:322). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P18 (SEQ ID NO:377) also has the following non-silent SNPs (Single Nucleotide Polymorphisms) as listed in Table 29, (given according to their position(s) on the amino acid sequence, with the alternative amino acid(s) listed; the last column indicates whether the SNP is known or not; the presence of known SNPs in variant protein HUMLYSYL_PEA_{—}1_P18 (SEQ ID NO:377) Sequence provides support for the deduced sequence of this variant protein according to the present invention).

TABLE 29
Amino acid mutations

| SNP position(s) on amino acid | Alternative amino | Previously known |
|---|---|---|
| sequence | acid(s) | SNP? |
| 74 | L -> | No |
| 77 | R -> G | Yes |
| 79 | P -> | No |
| 120 | S -> | No |
| 120 | S -> F | No |
| 127 | P -> | No |

Variant protein HUMLYSYL_PEA_{—}1_P18 (SEQ ID NO:377) is encoded by the following transcript(s): HUMLYSYL_PEA_{—}1_T24 (SEQ ID NO:322), for which the sequence(s) is/are given at the end of the application. The coding portion of transcript HUMLYSYL_PEA_{—}1_T24 (SEQ ID NO:322) is shown in bold; this coding portion starts at position 104 and ends at position 514. The transcript also has the following SNPs as listed in Table 30 (given according to their position on the nucleotide sequence, with the alternative nucleic acid listed; the last column indicates whether the SNP is known or not; the presence of known SNPs in variant protein HUMLYSYL_PEA_{—}1_P18 (SEQ ID NO:377) Sequence provides support for the deduced sequence of this variant protein according to the present invention).

TABLE 30
Nucleic acid SNPs

| SNP position on nucleotide | Alternative nucleic | Previously known |
|---|---|---|
| sequence | acid | SNP? |
| 37 | C -> | No |
| 71 | C -> | No |
| 102 | C -> | No |
| 217 | C -> A | Yes |
| 325 | G -> | No |
| 332 | C -> G | Yes |
| 338 | C -> | No |
| 462 | C -> | No |
| 462 | C -> T | No |
| 482 | C -> | No |
| 519 | G -> A | No |
| 558 | C -> | No |
| 558 | C -> G | No |
| 572 | C -> T | No |
| 645 | G -> A | No |
| 645 | G -> C | No |
| 685 | G -> | No |
| 692 | C -> T | Yes |
| 701 | G -> C | Yes |
| 794 | C -> | No |
| 808 | G -> C | Yes |
| 860 | C -> T | Yes |
| 875 | G -> T | Yes |
| 931 | C -> A | Yes |

Variant protein HUMLYSYL_PEA_{—}1_P24 (SEQ ID NO:378) according to the present invention has an amino acid sequence as given at the end of the application; it is encoded by transcript(s) HUMLYSYL_PEA_{—}1_T8 (SEQ ID NO:317). An alignment is given to the known protein (Procollagen-lysine,2-oxoglutarate 5-dioxygenase 1 precursor (SEQ ID NO:367)) at the end of the application. One or more alignments to one or more previously published protein sequences are given at the end of the application. A brief description of the relationship of the variant protein according to the present invention to each such aligned protein is as follows:

Comparison report between HUMLYSYL_PEA_{—}1_P24 (SEQ ID NO:378) and PLO1_HUMAN_V1 (SEQ ID NO:368):

1. An isolated chimeric polypeptide encoding for HUMLYSYL_PEA_{—}1_P24 (SEQ ID NO:378), comprising a first amino acid sequence being at least 90% homologous to MRPLLLALLGWLLLAEAKGDAKPEDNLLVLTVATKETEGFRRFKRSAQFFNYKIQAL GLGEDWNVEKGTSAGGGQKVRLLKKALEKHADKEDLVILFADSYDVLFASGPRELLK KFRQARSQVVFSAEELIYPDRRLETKYPVVSDGKRFLGSGGFIGYAPNLSKLVAEWEGQ DSDSDQLFYTKIFLDPEKR corresponding to amino acids 1-193 of PLO1_HUMAN_V1 (SEQ ID NO:368), which also corresponds to amino acids 1-193 of HUMLYSYL_PEA_{—}1_P24 (SEQ ID NO:378), and a second amino acid sequence being at least 70%, optionally at least 80%, preferably at least 85%, more preferably at least 90% and most preferably at least 95% homologous to a polypeptide having the sequence VSRLHS (SEQ ID NO:478) corresponding to amino acids 194-199 of HUMLYSYL_PEA_{—}1_P24 (SEQ ID NO:378), wherein said first amino acid sequence and second amino acid sequence are contiguous and in a sequential order.

2. An isolated polypeptide encoding for a tail of HUMLYSYL_PEA_{—}1_P24 (SEQ ID NO:378), comprising a polypeptide being at least 70%, optionally at least about 80%, preferably at least about 85%, more preferably at least about 90% and most preferably at least about 95% homologous to the sequence VSRLHS (SEQ ID NO:478) in HUMLYSYL_PEA_{—}1_P24 (SEQ ID NO:378).

It should be noted that the known protein sequence (PLO1_HUMAN (SEQ ID NO:367)) has one or more changes than the sequence given at the end of the application and named as being the amino acid sequence for PLO1_HUMAN_V1 (SEQ ID NO:368). These changes were previously known to occur and are listed in the table below.

TABLE 31
Changes to PLO1_HUMAN_V1 (SEQ ID NO: 368)

| SNP position(s) on | |
|---|---|
| amino acid sequence | Type of change |
| 100 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMLYSYL_PEA_{—}1_P24 (SEQ ID NO:378) also has the following non-silent SNPs (Single Nucleotide Polymorphisms) as listed in Table 32, (given according to their position(s) on the amino acid sequence, with the alternative amino acid(s) listed; the last column indicates whether the SNP is known or not; the presence of known SNPs in variant protein HUMLYSYL_PEA_{—}1_P24 (SEQ ID NO:378) Sequence provides support for the deduced sequence of this variant protein according to the present invention).

TABLE 32
Amino acid mutations

| SNP position(s) on amino acid | Alternative amino | Previously known |
|---|---|---|
| sequence | acid(s) | SNP? |
| 67 | E -> D | Yes |
| 98 | F -> | No |
| 99 | A -> T | Yes |
| 120 | A -> S | Yes |
| 178 | S -> | No |
| 179 | D -> N | No |

Variant protein HUMLYSYL_PEA_{—}1_P24 (SEQ ID NO:378) is encoded by the following transcript(s): HUMLYSYL_PEA_{—}1_T8 (SEQ ID NO:317), for which the sequence(s) is/are given at the end of the application. The coding portion of transcript HUMLYSYL_PEA_{—}1_T8 (SEQ ID NO:317) is shown in bold; this coding portion starts at position 104 and ends at position 700. The transcript also has the following SNPs as listed in Table 33 (given according to their position on the nucleotide sequence, with the alternative nucleic acid listed; the last column indicates whether the SNP is known or not; the presence of known SNPs in variant protein HUMLYSYL_PEA_{—}1_P24 (SEQ ID NO:378) Sequence provides support for the deduced sequence of this variant protein according to the present invention).

TABLE 33
Nucleic acid SNPs

| SNP position on nucleotide | | Previously |
|---|---|---|
| sequence | Alternative nucleic acid | known SNP? |
| 37 | C -> | No |
| 71 | C -> | No |
| 102 | C -> | No |
| 217 | C -> A | Yes |
| 304 | G -> C | Yes |
| 370 | A -> G | Yes |
| 397 | C -> | No |
| 397 | C -> T | Yes |
| 398 | G -> A | Yes |
| 461 | G -> T | Yes |
| 636 | G -> | No |
| 638 | G -> A | No |
| 820 | G -> A | Yes |
| 839 | G -> A | Yes |
| 971 | C -> | No |
| 1054 | C -> | No |
| 1054 | C -> G | No |
| 1287 | C -> T | Yes |
| 1500 | G -> A | Yes |
| 1516 | C -> | No |
| 1565 | C -> T | Yes |
| 1745 | G -> C | No |
| 1991 | A -> C | Yes |
| 2260 | G -> | No |
| 2291 | C -> T | Yes |
| 2297 | G -> C | Yes |
| 2321 | G -> T | Yes |
| 2483 | T -> C | Yes |
| 2492 | C -> G | Yes |
| 2503 | G -> | No |
| 2509 | C -> | No |
| 2607 | G -> | No |
| 2614 | C -> G | Yes |
| 2620 | C -> | No |
| 2744 | C -> | No |
| 2744 | C -> T | No |
| 2764 | C -> | No |
| 2801 | G -> A | No |
| 2840 | C -> | No |
| 2840 | C -> G | No |
| 2854 | C -> T | No |
| 2927 | G -> A | No |
| 2927 | G -> C | No |
| 2967 | G -> | No |
| 2974 | C -> T | Yes |
| 2983 | G -> C | Yes |
| 3076 | C -> | No |
| 3090 | G -> C | Yes |
| 3142 | C -> T | Yes |
| 3157 | G -> T | Yes |
| 3213 | C -> A | Yes |

As noted above, cluster HUMLYSYL features 44 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMLYSYL_PEA_{—}1_node_{—}6 (SEQ ID NO:323) according to the present invention is supported by 3 libraries. The number of libraries was determined as previously described. This segment can be found in the following transcript(s): HUMLYSYL_PEA_{—}1_T4 (SEQ ID NO:314). Table 34 below describes the starting and ending position of this segment on each transcript.

TABLE 34
Segment location on transcripts

| | Segment | Segment |
|---|---|---|
| Transcript name | starting position | ending position |
| HUMLYSYL_PEA_1_T4 (SEQ ID | 180 | 320 |
| NO: 314) | | |

Segment cluster HUMLYSYL_PEA_{—}1_node_{—}14 (SEQ ID NO:324) according to the present invention is supported by 122 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 35 below describes the starting and ending position of this segment on each transcript.

TABLE 35
Segment location on transcripts

| | | Segment |
|---|---|---|
| | Segment | ending |
| Transcript name | starting position | position |
| HUMLYSYL_PEA_1_T2 (SEQ ID | 406 | 569 |
| NO: 313) | | |
| HUMLYSYL_PEA_1_T4 (SEQ ID | 547 | 710 |
| NO: 314) | | |
| HUMLYSYL_PEA_1_T5 (SEQ ID | 406 | 569 |
| NO: 315) | | |
| HUMLYSYL_PEA_1_T6 (SEQ ID | 433 | 596 |
| NO: 316) | | |
| HUMLYSYL_PEA_1_T8 (SEQ ID | 406 | 569 |
| NO: 317) | | |
| HUMLYSYL_PEA_1_T9 (SEQ ID | 406 | 569 |
| NO: 318) | | |
| HUMLYSYL_PEA_1_T19 (SEQ ID | 406 | 569 |
| NO: 319) | | |
| HUMLYSYL_PEA_1_T20 (SEQ ID | 406 | 569 |
| NO: 320) | | |
| HUMLYSYL_PEA_1_T22 (SEQ ID | 406 | 569 |
| NO: 321) | | |

Segment cluster HUMLYSYL_PEA_{—}1_node_{—}19 (SEQ ID NO:325) according to the present invention is supported by 4 libraries. The number of libraries was determined as previously described. This segment can be found in the following transcript(s): HUMLYSYL_PEA_{—}1_T8 (SEQ ID NO:317). Table 36 below describes the starting and ending position of this segment on each transcript.

TABLE 36
Segment location on transcripts

| | Segment | Segment |
|---|---|---|
| Transcript name | starting position | ending position |
| HUMLYSYL_PEA_1_T8 (SEQ ID | 683 | 938 |
| NO: 317) | | |

Segment cluster HUMLYSYL_PEA_{—}1_node_{—}38 (SEQ ID NO:326) according to the present invention is supported by 94 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 37 below describes the starting and ending position of this segment on each transcript.

TABLE 37
Segment location on transcripts

| | | Segment |
|---|---|---|
| | Segment | ending |
| Transcript name | starting position | position |
| HUMLYSYL_PEA_1_T2 (SEQ ID | 1306 | 1431 |
| NO: 313) | | |
| HUMLYSYL_PEA_1_T4 (SEQ ID | 1447 | 1572 |
| NO: 314) | | |
| HUMLYSYL_PEA_1_T5 (SEQ ID | 1231 | 1356 |
| NO: 315) | | |
| HUMLYSYL_PEA_1_T6 (SEQ ID | 1333 | 1458 |
| NO: 316) | | |
| HUMLYSYL_PEA_1_T8 (SEQ ID | 1562 | 1687 |
| NO: 317) | | |
| HUMLYSYL_PEA_1_T9 (SEQ ID | 1312 | 1437 |
| NO: 318) | | |
| HUMLYSYL_PEA_1_T19 (SEQ ID | 1306 | 1431 |
| NO: 319) | | |
| HUMLYSYL_PEA_1_T20 (SEQ ID | 1306 | 1431 |
| NO: 320) | | |
| HUMLYSYL_PEA_1_T22 (SEQ ID | 1306 | 1431 |
| NO: 321) | | |

Segment cluster HUMLYSYL_PEA_{—}1_node_{—}55 (SEQ ID NO:327) according to the present invention is supported by 149 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317) and HUMLYSYL_PEA_{—}1_T9 (SEQ ID NO:318). Table 38 below describes the starting and ending position of this segment on each transcript.

TABLE 38
Segment location on transcripts

| | Segment | Segment |
|---|---|---|
| Transcript name | starting position | ending position |
| HUMLYSYL_PEA_1_T2 (SEQ ID | 1912 | 2040 |
| NO: 313) | | |
| HUMLYSYL_PEA_1_T4 (SEQ ID | 2000 | 2128 |
| NO: 314) | | |
| HUMLYSYL_PEA_1_T5 (SEQ ID | 1784 | 1912 |
| NO: 315) | | |
| HUMLYSYL_PEA_1_T6 (SEQ ID | 1886 | 2014 |
| NO: 316) | | |
| HUMLYSYL_PEA_1_T8 (SEQ ID | 2115 | 2243 |
| NO: 317) | | |
| HUMLYSYL_PEA_1_T9 (SEQ ID | 1865 | 1993 |
| NO: 318) | | |

Segment cluster HUMLYSYL_PEA_{—}1_node_{—}59 (SEQ ID NO:328) according to the present invention is supported by 161 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317) and HUMLYSYL_PEA_{—}1_T9 (SEQ ID NO:318). Table 39 below describes the starting and ending position of this segment on each transcript.

TABLE 39
Segment location on transcripts

| | Segment | Segment |
|---|---|---|
| Transcript name | starting position | ending position |
| HUMLYSYL_PEA_1_T2 (SEQ ID | 2059 | 2184 |
| NO: 313) | | |
| HUMLYSYL_PEA_1_T4 (SEQ ID | 2147 | 2272 |
| NO: 314) | | |
| HUMLYSYL_PEA_1_T5 (SEQ ID | 1931 | 2056 |
| NO: 315) | | |
| HUMLYSYL_PEA_1_T6 (SEQ ID | 2033 | 2158 |
| NO: 316) | | |
| HUMLYSYL_PEA_1_T8 (SEQ ID | 2262 | 2387 |
| NO: 317) | | |
| HUMLYSYL_PEA_1_T9 (SEQ ID | 2012 | 2137 |
| NO: 318) | | |

Segment cluster HUMLYSYL_PEA_{—}1_node_{—}61 (SEQ ID NO:329) according to the present invention is supported by 196 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_T8 (SEQ ID NO:317) and HUMLYSYL_PEA_{—}1_T9 (SEQ ID NO:318). Table 40 below describes the starting and ending position of this segment on each transcript.

TABLE 40
Segment location on transcripts

| | Segment | Segment |
|---|---|---|
| Transcript name | starting position | ending position |
| HUMLYSYL_PEA_1_T2 (SEQ ID | 2185 | 2350 |
| NO: 313) | | |
| HUMLYSYL_PEA_1_T4 (SEQ ID | 2273 | 2438 |
| NO: 314) | | |
| HUMLYSYL_PEA_1_T5 (SEQ ID | 2057 | 2222 |
| NO: 315) | | |
| HUMLYSYL_PEA_1_T6 (SEQ ID | 2159 | 2324 |
| NO: 316) | | |
| HUMLYSYL_PEA_1_T8 (SEQ ID | 2388 | 2553 |
| NO: 317) | | |
| HUMLYSYL_PEA_1_T9 (SEQ ID | 2138 | 2303 |
| NO: 318) | | |

Segment cluster HUMLYSYL_PEA_{—}1_node_{—}62 (SEQ ID NO:330) according to the present invention is supported by 275 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}_T9 (SEQ ID NO:318) and HUMLYSYL_PEA_{—}1_T24 (SEQ ID NO:322). Table 41 below describes the starting and ending position of this segment on each transcript.

TABLE 41
Segment location on transcripts

| | | Segment |
|---|---|---|
| | Segment | ending |
| Transcript name | starting position | position |
| HUMLYSYL_PEA_1_T2 (SEQ ID | 2351 | 2622 |
| NO: 313) | | |
| HUMLYSYL_PEA_1_T4 (SEQ ID | 2439 | 2710 |
| NO: 314) | | |
| HUMLYSYL_PEA_1_T5 (SEQ ID | 2223 | 2494 |
| NO: 315) | | |
| HUMLYSYL_PEA_1_T6 (SEQ ID | 2325 | 2596 |
| NO: 316) | | |
| HUMLYSYL_PEA_1_T8 (SEQ ID | 2554 | 2825 |
| NO: 317) | | |
| HUMLYSYL_PEA_1_T9 (SEQ ID | 2304 | 2575 |
| NO: 318) | | |
| HUMLYSYL_PEA_1_T24 (SEQ ID | 272 | 543 |
| NO: 322) | | |

Segment cluster HUMLYSYL_PEA_{—}1_node_{—}65 (SEQ ID NO:331) according to the present invention is supported by 233 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318) and HUMLYSYL_PEA_{—}1_T24 (SEQ ID NO:322). Table 42 below describes the starting and ending position of this segment on each transcript.

TABLE 42
Segment location on transcripts

| | | Segment |
|---|---|---|
| | Segment | ending |
| Transcript name | starting position | position |
| HUMLYSYL_PEA_1_T2 (SEQ ID | 2675 | 2814 |
| NO: 313) | | |
| HUMLYSYL_PEA_1_T4 (SEQ ID | 2763 | 2902 |
| NO: 314) | | |
| HUMLYSYL_PEA_1_T5 (SEQ ID | 2547 | 2686 |
| NO: 315) | | |
| HUMLYSYL_PEA_1_T6 (SEQ ID | 2649 | 2788 |
| NO: 316) | | |
| HUMLYSYL_PEA_1_T8 (SEQ ID | 2878 | 3017 |
| NO: 317) | | |
| HUMLYSYL_PEA_1_T9 (SEQ ID | 2628 | 2767 |
| NO: 318) | | |
| HUMLYSYL_PEA_1_T24 (SEQ ID | 596 | 735 |
| NO: 322) | | |

Segment cluster HUMLYSYL_PEA_{—}1_node_{—}71 (SEQ ID NO:332) according to the present invention is supported by 187 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T24 (SEQ ID NO:322). Table 43 below describes the starting and ending position of this segment on each transcript.

TABLE 43
Segment location on transcripts

| | Segment | |
|---|---|---|
| | starting | Segment |
| Transcript name | position | ending position |
| HUMLYSYL_PEA_1_T2 (SEQ ID | 2895 | 3027 |
| NO: 313) | | |
| HUMLYSYL_PEA_1_T4 (SEQ ID | 2983 | 3115 |
| NO: 314) | | |
| HUMLYSYL_PEA_1_T5 (SEQ ID | 2767 | 2899 |
| NO: 315) | | |
| HUMLYSYL_PEA_1_T6 (SEQ ID | 2869 | 3001 |
| NO: 316) | | |
| HUMLYSYL_PEA_1_T8 (SEQ ID | 3098 | 3230 |
| NO: 317) | | |
| HUMLYSYL_PEA_1_T9 (SEQ ID | 2848 | 2980 |
| NO: 318) | | |
| HUMLYSYL_PEA_1_T19 (SEQ ID | 1939 | 2071 |
| NO: 319) | | |
| HUMLYSYL_PEA_1_T20 (SEQ ID | 1872 | 2004 |
| NO: 320) | | |
| HUMLYSYL_PEA_1_T24 (SEQ ID | 816 | 948 |
| NO: 322) | | |

Segment cluster HUMLYSYL_PEA_{—}1_node_{—}72 (SEQ ID NO:333) according to the present invention is supported by 143 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T24 (SEQ ID NO:322). Table 44 below describes the starting and ending position of this segment on each transcript.

TABLE 44
Segment location on transcripts

| | Segment | |
|---|---|---|
| | starting | Segment |
| Transcript name | position | ending position |
| HUMLYSYL_PEA_1_T2 (SEQ ID | 3028 | 3069 |
| NO: 313) | | |
| HUMLYSYL_PEA_1_T4 (SEQ ID | 3116 | 3157 |
| NO: 314) | | |
| HUMLYSYL_PEA_1_T5 (SEQ ID | 2900 | 2941 |
| NO: 315) | | |
| HUMLYSYL_PEA_1_T6 (SEQ ID | 3002 | 3043 |
| NO: 316) | | |
| HUMLYSYL_PEA_1_T8 (SEQ ID | 3231 | 3272 |
| NO: 317) | | |
| HUMLYSYL_PEA_1_T9 (SEQ ID | 2981 | 3022 |
| NO: 318) | | |
| HUMLYSYL_PEA_1_T19 (SEQ ID | 2072 | 2113 |
| NO: 319) | | |
| HUMLYSYL_PEA_1_T20 (SEQ ID | 2005 | 2046 |
| NO: 320) | | |
| HUMLYSYL_PEA_1_T24 (SEQ ID | 949 | 990 |
| NO: 322) | | |

According to an optional embodiment of the present invention, short segments related to the above cluster are also provided. These segments are up to about 120 bp in length, and so are included in a separate description.

Segment cluster HUMLYSYL_PEA_{—}1_node_{—}3 (SEQ ID NO:334) according to the present invention is supported by 68 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL-PEA-1_T19 (SEQ ID NO:319), HUMLYSYL PEA_{—}1_T20 (SEQ ID NO:320), HUMLYSYL_PEA_{—}1_T22 (SEQ ID NO:321) and HUMLYSYL_PEA_{—}1_T24 (SEQ ID NO:322). Table 45 below describes the starting and ending position of this segment on each transcript.

TABLE 45
Segment location on transcripts

| | Segment | |
|---|---|---|
| | starting | Segment |
| Transcript name | position | ending position |
| HUMLYSYL_PEA_1_T2 (SEQ ID | 1 | 76 |
| NO: 313) | | |
| HUMLYSYL_PEA_1_T4 (SEQ ID | 1 | 76 |
| NO: 314) | | |
| HUMLYSYL_PEA_1_T5 (SEQ ID | 1 | 76 |
| NO: 315) | | |
| HUMLYSYL_PEA_1_T6 (SEQ ID | 1 | 76 |
| NO: 316) | | |
| HUMLYSYL_PEA_1_T8 (SEQ ID | 1 | 76 |
| NO: 317) | | |
| HUMLYSYL_PEA_1_T9 (SEQ ID | 1 | 76 |
| NO: 318) | | |
| HUMLYSYL_PEA_1_T19 (SEQ ID | 1 | 76 |
| NO: 319) | | |
| HUMLYSYL_PEA_1_T20 (SEQ ID | 1 | 76 |
| NO: 320) | | |
| HUMLYSYL_PEA_1_T22 (SEQ ID | 1 | 76 |
| NO: 321) | | |
| HUMLYSYL_PEA_1_T24 (SEQ ID | 1 | 76 |
| NO: 322) | | |

Segment cluster HUMLYSYL_PEA_{—}1_node_{—}4 (SEQ ID NO:335) according to the present invention is supported by 99 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320), HUMLYSYL_PEA_{—}1_T22 (SEQ ID NO:321) and HUMLYSYL_PEA_{—}1_T24 (SEQ ID NO:322). Table 46 below describes the starting and ending position of this segment on each transcript.

TABLE 46
Segment location on transcripts

| | | |
|---|---|---|
| | Segment | |
| | starting | Segment |
| Transcript name | position | ending position |

| | | |
|---|---|---|
| HUMLYSYL_PEA_1_T2 (SEQ ID | 77 | 179 |
| NO: 313) | | |
| HUMLYSYL_PEA_1_T4 (SEQ ID | 77 | 179 |
| NO: 314) | | |
| HUMLYSYL_PEA_1_T5 (SEQ ID | 77 | 179 |
| NO: 315) | | |
| HUMLYSYL_PEA_1_T6 (SEQ ID | 77 | 179 |
| NO: 316) | | |
| HUMLYSYL_PEA_1_T8 (SEQ ID | 77 | 179 |
| NO: 317) | | |
| HUMLYSYL_PEA_1_T9 (SEQ ID | 77 | 179 |
| NO: 318) | | |
| HUMLYSYL_PEA_1_T19 (SEQ ID | 77 | 179 |
| NO: 319) | | |
| HUMLYSYL_PEA_1_T20 (SEQ ID | 77 | 179 |
| NO: 320) | | |
| HUMLYSYL_PEA_1_T22 (SEQ ID | 77 | 179 |
| NO: 321) | | |
| HUMLYSYL_PEA_1_T24 (SEQ ID | 77 | 179 |
| NO: 322) | | |

Segment cluster HUMLYSYL_PEA_{—}1_node_{—}8 (SEQ ID NO:336) according to the present invention is supported by 108 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320), HUMLYSYL_PEA_{—}1_T22 (SEQ ID NO:321) and HUMLYSYL_PEA_{—}1_T24 (SEQ ID NO:322). Table 47 below describes the starting and ending position of this segment on each transcript.

TABLE 47
Segment location on transcripts

| | Segment | |
|---|---|---|
| | starting | Segment |
| Transcript name | position | ending position |
| HUMLYSYL_PEA_1_T2 (SEQ ID | 180 | 271 |
| NO: 313) | | |
| HUMLYSYL_PEA_1_T4 (SEQ ID | 321 | 412 |
| NO: 314) | | |
| HUMLYSYL_PEA_1_T5 (SEQ ID | 180 | 271 |
| NO: 315) | | |
| HUMLYSYL_PEA_1_T6 (SEQ ID | 180 | 271 |
| NO: 316) | | |
| HUMLYSYL_PEA_1_T8 (SEQ ID | 180 | 271 |
| NO: 317) | | |
| HUMLYSYL_PEA_1_T9 (SEQ ID | 180 | 271 |
| NO: 318) | | |
| HUMLYSYL_PEA_1_T19 (SEQ ID | 180 | 271 |
| NO: 319) | | |
| HUMLYSYL_PEA_1_T20 (SEQ ID | 180 | 271 |
| NO: 320) | | |
| HUMLYSYL_PEA_1_T22 (SEQ ID | 180 | 271 |
| NO: 321) | | |
| HUMLYSYL_PEA_1_T24 (SEQ ID | 180 | 271 |
| NO: 322) | | |

Segment cluster HUMLYSYL_PEA_{—}1_node_{—}10 (SEQ ID NO:337) according to the present invention is supported by 1 libraries. The number of libraries was determined as previously described. This segment can be found in the following transcript(s): HUMLYSYL_PEA_{—}1_T6 (SEQ ID NO:316). Table 48 below describes the starting and ending position of this segment on each transcript.

TABLE 48
Segment location on transcripts

| | Segment | Segment |
|---|---|---|
| Transcript name | starting position | ending position |
| HUMLYSYL_PEA_1_T6 (SEQ ID | 272 | 298 |
| NO: 316) | | |

Segment cluster HUMLYSYL_PEA_{—}1_node_{—}11 (SEQ ID NO:338) according to the present invention is supported by 120 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 49 below describes the starting and ending position of this segment on each transcript.

TABLE 49
Segment location on transcripts

| | Segment | |
|---|---|---|
| | starting | Segment |
| Transcript name | position | ending position |
| HUMLYSYL_PEA_1_T2 (SEQ ID | 272 | 355 |
| NO: 313) | | |
| HUMLYSYL_PEA_1_T4 (SEQ ID | 413 | 496 |
| NO: 314) | | |
| HUMLYSYL_PEA_1_T5 (SEQ ID | 272 | 355 |
| NO: 315) | | |
| HUMLYSYL_PEA_1_T6 (SEQ ID | 299 | 382 |
| NO: 316) | | |
| HUMLYSYL_PEA_1_T8 (SEQ ID | 272 | 355 |
| NO: 317) | | |
| HUMLYSYL_PEA_1_T9 (SEQ ID | 272 | 355 |
| NO: 318) | | |
| HUMLYSYL_PEA_1_T19 (SEQ ID | 272 | 355 |
| NO: 319) | | |
| HUMLYSYL_PEA_1_T20 (SEQ ID | 272 | 355 |
| NO: 320) | | |
| HUMLYSYL_PEA_1_T22 (SEQ ID | 272 | 355 |
| NO: 321) | | |

Segment cluster HUMLYSYL_PEA_{—}1_node_{—}12 (SEQ ID NO:339) according to the present invention is supported by 111 libraries. The number of libraries was determined as previously described. This segment can be found in the following transcript(s): HUMLYSYL_PEA_T2 (SEQ ID NO:313), HUMLYSYL PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 50 below describes the starting and ending position of this segment on each transcript.

TABLE 50
Segment location on transcripts

| | Segment | |
|---|---|---|
| | starting | Segment |
| Transcript name | position | ending position |
| HUMLYSYL_PEA_1_T2 (SEQ ID | 356 | 405 |
| NO: 313) | | |
| HUMLYSYL_PEA_1_T4 (SEQ ID | 497 | 546 |
| NO: 314) | | |
| HUMLYSYL_PEA_1_T5 (SEQ ID | 356 | 405 |
| NO: 315) | | |
| HUMLYSYL_PEA_1_T6 (SEQ ID | 383 | 432 |
| NO: 316) | | |
| HUMLYSYL_PEA_1_T8 (SEQ ID | 356 | 405 |
| NO: 317) | | |
| HUMLYSYL_PEA_1_T9 (SEQ ID | 356 | 405 |
| NO: 318) | | |
| HUMLYSYL_PEA_1_T19 (SEQ ID | 356 | 405 |
| NO: 319) | | |
| HUMLYSYL_PEA_1_T20 (SEQ ID | 356 | 405 |
| NO: 320) | | |
| HUMLYSYL_PEA_1_T22 (SEQ ID | 356 | 405 |
| NO: 321) | | |

Segment cluster HUMLYSYL_PEA_{—}1_node_{—}16 (SEQ ID NO:340) according to the present invention is supported by 127 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 51 below describes the starting and ending position of this segment on each transcript.

TABLE 51
Segment location on transcripts

| | Segment | |
|---|---|---|
| | starting | Segment |
| Transcript name | position | ending position |
| HUMLYSYL_PEA_1_T2 (SEQ ID | 570 | 682 |
| NO: 313) | | |
| HUMLYSYL_PEA_1_T4 (SEQ ID | 711 | 823 |
| NO: 314) | | |
| HUMLYSYL_PEA_1_T5 (SEQ ID | 570 | 682 |
| NO: 315) | | |
| HUMLYSYL_PEA_1_T6 (SEQ ID | 597 | 709 |
| NO: 316) | | |
| HUMLYSYL_PEA_1_T8 (SEQ ID | 570 | 682 |
| NO: 317) | | |
| HUMLYSYL_PEA_1_T9 (SEQ ID | 570 | 682 |
| NO: 318) | | |
| HUMLYSYL_PEA_1_T19 (SEQ ID | 570 | 682 |
| NO: 319) | | |
| HUMLYSYL_PEA_1_T20 (SEQ ID | 570 | 682 |
| NO: 320) | | |
| HUMLYSYL_PEA_1_T22 (SEQ ID | 570 | 682 |
| NO: 321) | | |

Segment cluster HUMLYSYL_PEA_{—}1_node_{—}20 (SEQ ID NO:341) according to the present invention is supported by 107 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 52 below describes the starting and ending position of this segment on each transcript.

TABLE 52
Segment location on transcripts

| | Segment | |
|---|---|---|
| | starting | Segment |
| Transcript name | position | ending position |
| HUMLYSYL_PEA_1_T2 (SEQ ID | 683 | 746 |
| NO: 313) | | |
| HUMLYSYL_PEA_1_T4 (SEQ ID | 824 | 887 |
| NO: 314) | | |
| HUMLYSYL_PEA_1_T5 (SEQ ID | 683 | 746 |
| NO: 315) | | |
| HUMLYSYL_PEA_1_T6 (SEQ ID | 710 | 773 |
| NO: 316) | | |
| HUMLYSYL_PEA_1_T8 (SEQ ID | 939 | 1002 |
| NO: 317) | | |
| HUMLYSYL_PEA_1_T9 (SEQ ID | 683 | 746 |
| NO: 318) | | |
| HUMLYSYL_PEA_1_T19 (SEQ ID | 683 | 746 |
| NO: 319) | | |
| HUMLYSYL_PEA_1_T20 (SEQ ID | 683 | 746 |
| NO: 320) | | |
| HUMLYSYL_PEA_1_T22 (SEQ ID | 683 | 746 |
| NO: 321) | | |

Segment cluster HUMLYSYL_PEA_{—}1_node_{—}23 (SEQ ID NO:342) according to the present invention is supported by 111 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 53 below describes the starting and ending position of this segment on each transcript.

TABLE 53
Segment location on transcripts

| | Segment | |
|---|---|---|
| | starting | Segment |
| Transcript name | position | ending position |
| HUMLYSYL_PEA_1_T2 (SEQ ID | 747 | 844 |
| NO: 313) | | |
| HUMLYSYL_PEA_1_T4 (SEQ ID | 888 | 985 |
| NO: 314) | | |
| HUMLYSYL_PEA_1_T5 (SEQ ID | 747 | 844 |
| NO: 315) | | |
| HUMLYSYL_PEA_1_T6 (SEQ ID | 774 | 871 |
| NO: 316) | | |
| HUMLYSYL_PEA_1_T8 (SEQ ID | 1003 | 1100 |
| NO: 317) | | |
| HUMLYSYL_PEA_1_T19 (SEQ ID | 747 | 844 |
| NO: 319) | | |
| HUMLYSYL_PEA_1_T20 (SEQ ID | 747 | 844 |
| NO: 320) | | |
| HUMLYSYL_PEA_1_T22 (SEQ ID | 747 | 844 |
| NO: 321) | | |

Segment cluster HUMLYSYL_PEA_{—}1_node_{—}25 (SEQ ID NO:343) according to the present invention is supported by 1 libraries. The number of libraries was determined as previously described. This segment can be found in the following transcript(s): HUMLYSYL_PEA_{—}1_T9 (SEQ ID NO:318). Table 54 below describes the starting and ending position of this segment on each transcript.

TABLE 54
Segment location on transcripts

| | Segment | |
|---|---|---|
| | starting | Segment |
| Transcript name | position | ending position |
| HUMLYSYL_PEA_1_T9 (SEQ ID | 747 | 850 |
| NO: 318) | | |

Segment cluster HUMLYSYL_PEA_{—}1_node_{—}28 (SEQ ID NO:344) according to the present invention is supported by 105 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 55 below describes the starting and ending position of this segment on each transcript.

TABLE 55
Segment location on transcripts

| | Segment | |
|---|---|---|
| | starting | Segment |
| Transcript name | position | ending position |
| HUMLYSYL_PEA_1_T2 (SEQ ID | 845 | 946 |
| NO: 313) | | |
| HUMLYSYL_PEA_1_T4 (SEQ ID | 986 | 1087 |
| NO: 314) | | |
| HUMLYSYL_PEA_1_T5 (SEQ ID | 845 | 946 |
| NO: 315) | | |
| HUMLYSYL_PEA_1_T6 (SEQ ID | 872 | 973 |
| NO: 316) | | |
| HUMLYSYL_PEA_1_T8 (SEQ ID | 1101 | 1202 |
| NO: 317) | | |
| HUMLYSYL_PEA_1_T9 (SEQ ID | 851 | 952 |
| NO: 318) | | |
| HUMLYSYL_PEA_1_T19 (SEQ ID | 845 | 946 |
| NO: 319) | | |
| HUMLYSYL_PEA_1_T20 (SEQ ID | 845 | 946 |
| NO: 320) | | |
| HUMLYSYL_PEA_1_T22 (SEQ ID | 845 | 946 |
| NO: 321) | | |

Segment cluster HUMLYSYL_PEA_{—}1_node_{—}30 (SEQ ID NO:345) according to the present invention is supported by 86 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 56 below describes the starting and ending position of this segment on each transcript.

TABLE 56
Segment location on transcripts

| | Segment | |
|---|---|---|
| | starting | Segment |
| Transcript name | position | ending position |
| HUMLYSYL_PEA_1_T2 (SEQ ID | 947 | 1021 |
| NO: 313) | | |
| HUMLYSYL_PEA_1_T4 (SEQ ID | 1088 | 1162 |
| NO: 314) | | |
| HUMLYSYL_PEA_1_T6 (SEQ ID | 974 | 1048 |
| NO: 316) | | |
| HUMLYSYL_PEA_1_T8 (SEQ ID | 1203 | 1277 |
| NO: 317) | | |
| HUMLYSYL_PEA_1_T9 (SEQ ID | 953 | 1027 |
| NO: 318) | | |
| HUMLYSYL_PEA_1_T19 (SEQ ID | 947 | 1021 |
| NO: 319) | | |
| HUMLYSYL_PEA_1_T20 (SEQ ID | 947 | 1021 |
| NO: 320) | | |
| HUMLYSYL_PEA_1_T22 (SEQ ID | 947 | 1021 |
| NO: 321) | | |

Segment cluster HUMLYSYL_PEA_{—}1_node_{—}31 (SEQ ID NO:346) according to the present invention is supported by 79 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 57 below describes the starting and ending position of this segment on each transcript.

TABLE 57
Segment location on transcripts

| | Segment | |
|---|---|---|
| | starting | Segment |
| Transcript name | position | ending position |
| HUMLYSYL_PEA_1_T2 (SEQ ID | 1022 | 1078 |
| NO: 313) | | |
| HUMLYSYL_PEA_1_T4 (SEQ ID | 1163 | 1219 |
| NO: 314) | | |
| HUMLYSYL_PEA_1_T5 (SEQ ID | 947 | 1003 |
| NO: 315) | | |
| HUMLYSYL_PEA_1_T6 (SEQ ID | 1049 | 1105 |
| NO: 316) | | |
| HUMLYSYL_PEA_1_T8 (SEQ ID | 1278 | 1334 |
| NO: 317) | | |
| HUMLYSYL_PEA_1_T9 (SEQ ID | 1028 | 1084 |
| NO: 318) | | |
| HUMLYSYL_PEA_1_T19 (SEQ ID | 1022 | 1078 |
| NO: 319) | | |
| HUMLYSYL_PEA_1_T20 (SEQ ID | 1022 | 1078 |
| NO: 320) | | |
| HUMLYSYL_PEA_1_T22 (SEQ ID | 1022 | 1078 |
| NO: 321) | | |

Segment cluster HUMLYSYL_PEA_{—}1_node_{—}33 (SEQ ID NO:347) according to the present invention is supported by 81 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 58 below describes the starting and ending position of this segment on each transcript.

TABLE 58
Segment location on transcripts

| | Segment | |
|---|---|---|
| | starting | Segment |
| Transcript name | position | ending position |
| HUMLYSYL_PEA_1_T2 (SEQ ID | 1079 | 1162 |
| NO: 313) | | |
| HUMLYSYL_PEA_1_T4 (SEQ ID | 1220 | 1303 |
| NO: 314) | | |
| HUMLYSYL_PEA_1_T5 (SEQ ID | 1004 | 1087 |
| NO: 315) | | |
| HUMLYSYL_PEA_1_T6 (SEQ ID | 1106 | 1189 |
| NO: 316) | | |
| HUMLYSYL_PEA_1_T8 (SEQ ID | 1335 | 1418 |
| NO: 317) | | |
| HUMLYSYL_PEA_1_T9 (SEQ ID | 1085 | 1168 |
| NO: 318) | | |
| HUMLYSYL_PEA_1_T19 (SEQ ID | 1079 | 1162 |
| NO: 319) | | |
| HUMLYSYL_PEA_1_T20 (SEQ ID | 1079 | 1162 |
| NO: 320) | | |
| HUMLYSYL_PEA_1_T22 (SEQ ID | 1079 | 1162 |
| NO: 321) | | |

Segment cluster HUMLYSYL_PEA_{—}1_node_{—}34 (SEQ ID NO:348) according to the present invention is supported by 74 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 59 below describes the starting and ending position of this segment on each transcript.

TABLE 59
Segment location on transcripts

| | Segment | |
|---|---|---|
| | starting | Segment |
| Transcript name | position | ending position |
| HUMLYSYL_PEA_1_T2 (SEQ ID | 1163 | 1200 |
| NO: 313) | | |
| HUMLYSYL_PEA_1_T4 (SEQ ID | 1304 | 1341 |
| NO: 314) | | |
| HUMLYSYL_PEA_1_T5 (SEQ ID | 1088 | 1125 |
| NO: 315) | | |
| HUMLYSYL_PEA_1_T6 (SEQ ID | 1190 | 1227 |
| NO: 316) | | |
| HUMLYSYL_PEA_1_T8 (SEQ ID | 1419 | 1456 |
| NO: 317) | | |
| HUMLYSYL_PEA_1_T9 (SEQ ID | 1169 | 1206 |
| NO: 318) | | |
| HUMLYSYL_PEA_1_T19 (SEQ ID | 1163 | 1200 |
| NO: 319) | | |
| HUMLYSYL_PEA_1_T20 (SEQ ID | 1163 | 1200 |
| NO: 320) | | |
| HUMLYSYL_PEA_1_T22 (SEQ ID | 1163 | 1200 |
| NO: 321) | | |

Segment cluster HUMLYSYL_PEA_{—}1_node_{—}36 (SEQ ID NO:349) according to the present invention is supported by 90 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 60 below describes the starting and ending position of this segment on each transcript.

TABLE 60
Segment location on transcripts

| | Segment | |
|---|---|---|
| | starting | Segment |
| Transcript name | position | ending position |
| HUMLYSYL_PEA_1_T2 (SEQ ID | 1201 | 1305 |
| NO: 313) | | |
| HUMLYSYL_PEA_1_T4 (SEQ ID | 1342 | 1446 |
| NO: 314) | | |
| HUMLYSYL_PEA_1_T5 (SEQ ID | 1126 | 1230 |
| NO: 315) | | |
| HUMLYSYL_PEA_1_T6 (SEQ ID | 1228 | 1332 |
| NO: 316) | | |
| HUMLYSYL_PEA_1_T8 (SEQ ID | 1457 | 1561 |
| NO: 317) | | |
| HUMLYSYL_PEA_1_T9 (SEQ ID | 1207 | 1311 |
| NO: 318) | | |
| HUMLYSYL_PEA_1_T19 (SEQ ID | 1201 | 1305 |
| NO: 319) | | |
| HUMLYSYL_PEA_1_T20 (SEQ ID | 1201 | 1305 |
| NO: 320) | | |
| HUMLYSYL_PEA_1_T22 (SEQ ID | 1201 | 1305 |
| NO: 321) | | |

Segment cluster HUMLYSYL_PEA_{—}1_node_{—}40 (SEQ ID NO:350) according to the present invention is supported by 96 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 61 below describes the starting and ending position of this segment on each transcript.

TABLE 61
Segment location on transcripts

| | Segment | |
|---|---|---|
| | starting | Segment |
| Transcript name | position | ending position |
| HUMLYSYL_PEA_1_T2 (SEQ ID | 1432 | 1468 |
| NO: 313) | | |
| HUMLYSYL_PEA_1_T4 (SEQ ID | 1573 | 1609 |
| NO: 314) | | |
| HUMLYSYL_PEA_1_T5 (SEQ ID | 1357 | 1393 |
| NO: 315) | | |
| HUMLYSYL_PEA_1_T6 (SEQ ID | 1459 | 1495 |
| NO: 316) | | |
| HUMLYSYL_PEA_1_T8 (SEQ ID | 1688 | 1724 |
| NO: 317) | | |
| HUMLYSYL_PEA_1_T9 (SEQ ID | 1438 | 1474 |
| NO: 318) | | |
| HUMLYSYL_PEA_1_T19 (SEQ ID | 1432 | 1468 |
| NO: 319) | | |
| HUMLYSYL_PEA_1_T20 (SEQ ID | 1432 | 1468 |
| NO: 320) | | |
| HUMLYSYL_PEA_1_T22 (SEQ ID | 1432 | 1468 |
| NO: 321) | | |

Segment cluster HUMLYSYL_PEA_{—}1_node_{—}41 (SEQ ID NO:351) according to the present invention is supported by 109 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 62 below describes the starting and ending position of this segment on each transcript.

TABLE 62
Segment location on transcripts

| | Segment | |
|---|---|---|
| | starting | Segment |
| Transcript name | position | ending position |
| HUMLYSYL_PEA_1_T2 (SEQ ID | 1469 | 1573 |
| NO: 313) | | |
| HUMLYSYL_PEA_1_T4 (SEQ ID | 1610 | 1714 |
| NO: 314) | | |
| HUMLYSYL_PEA_1_T5 (SEQ ID | 1394 | 1498 |
| NO: 315) | | |
| HUMLYSYL_PEA_1_T6 (SEQ ID | 1496 | 1600 |
| NO: 316) | | |
| HUMLYSYL_PEA_1_T8 (SEQ ID | 1725 | 1829 |
| NO: 317) | | |
| HUMLYSYL_PEA_1_T9 (SEQ ID | 1475 | 1579 |
| NO: 318) | | |
| HUMLYSYL_PEA_1_T19 (SEQ ID | 1469 | 1573 |
| NO: 319) | | |
| HUMLYSYL_PEA_1_T20 (SEQ ID | 1469 | 1573 |
| NO: 320) | | |
| HUMLYSYL_PEA_1_T22 (SEQ ID | 1469 | 1573 |
| NO: 321) | | |

Segment cluster HUMLYSYL_PEA_{—}1 node_{—}42 (SEQ ID NO:352) according to the present invention is supported by 1 libraries. The number of libraries was determined as previously described. This segment can be found in the following transcript(s): HUMLYSYL_PEA_{—}1_T2 (SEQ ID NO:313). Table 63 below describes the starting and ending position of this segment on each transcript.

TABLE 63
Segment location on transcripts

| | Segment | Segment |
|---|---|---|
| Transcript name | starting position | ending position |
| HUMLYSYL_PEA_1_T2 (SEQ ID | 1574 | 1626 |
| NO: 313) | | |

Segment cluster HUMLYSYL_PEA_{—}1_node_{—}44 (SEQ ID NO:353) according to the present invention can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 64 below describes the starting and ending position of this segment on each transcript.

TABLE 64
Segment location on transcripts

| | | Segment |
|---|---|---|
| | Segment | ending |
| Transcript name | starting position | position |
| HUMLYSYL_PEA_1_T2 (SEQ ID | 1627 | 1646 |
| NO: 313) | | |
| HUMLYSYL_PEA_1_T4 (SEQ ID | 1715 | 1734 |
| NO: 314) | | |
| HUMLYSYL_PEA_1_T5 (SEQ ID | 1499 | 1518 |
| NO: 315) | | |
| HUMLYSYL_PEA_1_T6 (SEQ ID | 1601 | 1620 |
| NO: 316) | | |
| HUMLYSYL_PEA_1_T8 (SEQ ID | 1830 | 1849 |
| NO: 317) | | |
| HUMLYSYL_PEA_1_T9 (SEQ ID | 1580 | 1599 |
| NO: 318) | | |
| HUMLYSYL_PEA_1_T19 (SEQ ID | 1574 | 1593 |
| NO: 319) | | |
| HUMLYSYL_PEA_1_T20 (SEQ ID | 1574 | 1593 |
| NO: 320) | | |
| HUMLYSYL_PEA_1_T22 (SEQ ID | 1574 | 1593 |
| NO: 321) | | |

Segment cluster HUMLYSYL_PEA_{—}1_node_{—}45 (SEQ ID NO:354) according to the present invention is supported by 99 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 65 below describes the starting and ending position of this segment on each transcript.

TABLE 65
Segment location on transcripts

| | | Segment |
|---|---|---|
| | Segment | ending |
| Transcript name | starting position | position |
| HUMLYSYL_PEA_1_T2 (SEQ ID | 1647 | 1685 |
| NO: 313) | | |
| HUMLYSYL_PEA_1_T4 (SEQ ID | 1735 | 1773 |
| NO: 314) | | |
| HUMLYSYL_PEA_1_T5 (SEQ ID | 1519 | 1557 |
| NO: 315) | | |
| HUMLYSYL_PEA_1_T6 (SEQ ID | 1621 | 1659 |
| NO: 316) | | |
| HUMLYSYL_PEA_1_T8 (SEQ ID | 1850 | 1888 |
| NO: 317) | | |
| HUMLYSYL_PEA_1_T9 (SEQ ID | 1600 | 1638 |
| NO: 318) | | |
| HUMLYSYL_PEA_1_T19 (SEQ ID | 1594 | 1632 |
| NO: 319) | | |
| HUMLYSYL_PEA_1_T20 (SEQ ID | 1594 | 1632 |
| NO: 320) | | |
| HUMLYSYL_PEA_1_T22 (SEQ ID | 1594 | 1632 |
| NO: 321) | | |

Segment cluster HUMLYSYL_PEA_{—}1_node_{—}46 (SEQ ID NO:355) according to the present invention is supported by 106 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—1}_T22 (SEQ ID NO:321). Table 66 below describes the starting and ending position of this segment on each transcript.

TABLE 66
Segment location on transcripts

| | | Segment |
|---|---|---|
| | Segment | ending |
| Transcript name | starting position | position |
| HUMLYSYL_PEA_1_T2 (SEQ ID | 1686 | 1740 |
| NO: 313) | | |
| HUMLYSYL_PEA_1_T4 (SEQ ID | 1774 | 1828 |
| NO: 314) | | |
| HUMLYSYL_PEA_1_T5 (SEQ ID | 1558 | 1612 |
| NO: 315) | | |
| HUMLYSYL_PEA_1_T6 (SEQ ID | 1660 | 1714 |
| NO: 316) | | |
| HUMLYSYL_PEA_1_T8 (SEQ ID | 1889 | 1943 |
| NO: 317) | | |
| HUMLYSYL_PEA_1_T9 (SEQ ID | 1639 | 1693 |
| NO: 318) | | |
| HUMLYSYL_PEA_1_T19 (SEQ ID | 1633 | 1687 |
| NO: 319) | | |
| HUMLYSYL_PEA_1_T20 (SEQ ID | 1633 | 1687 |
| NO: 320) | | |
| HUMLYSYL_PEA_1_T22 (SEQ ID | 1633 | 1687 |
| NO: 321) | | |

Segment cluster HUMLYSYL_PEA_{—}1_node_{—}48 (SEQ ID NO:356) according to the present invention is supported by 116 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T22 (SEQ ID NO:321). Table 67 below describes the starting and ending position of this segment on each transcript.

TABLE 67
Segment location on transcripts

| | | Segment |
|---|---|---|
| | Segment | ending |
| Transcript name | starting position | position |
| HUMLYSYL_PEA_1_T2 (SEQ ID | 1741 | 1806 |
| NO: 313) | | |
| HUMLYSYL_PEA_1_T4 (SEQ ID | 1829 | 1894 |
| NO: 314) | | |
| HUMLYSYL_PEA_1_T5 (SEQ ID | 1613 | 1678 |
| NO: 315) | | |
| HUMLYSYL_PEA_1_T6 (SEQ ID | 1715 | 1780 |
| NO: 316) | | |
| HUMLYSYL_PEA_1_T8 (SEQ ID | 1944 | 2009 |
| NO: 317) | | |
| HUMLYSYL_PEA_1_T9 (SEQ ID | 1694 | 1759 |
| NO: 318) | | |
| HUMLYSYL_PEA_1_T19 (SEQ ID | 1688 | 1753 |
| NO: 319) | | |
| HUMLYSYL_PEA_1_T20 (SEQ ID | 1688 | 1753 |
| NO: 320) | | |
| HUMLYSYL_PEA_1_T22 (SEQ ID | 1688 | 1753 |
| NO: 321) | | |

Segment cluster HUMLYSYL_PEA_{—}1_node_{—}49 (SEQ ID NO:357) according to the present invention is supported by 1 libraries. The number of libraries was determined as previously described. This segment can be found in the following transcript(s): HUMLYSYL_PEA_{—}1_T22 (SEQ ID NO:321). Table 68 below describes the starting and ending position of this segment on each transcript.

TABLE 68
Segment location on transcripts

| | | Segment |
|---|---|---|
| | Segment | ending |
| Transcript name | starting position | position |
| HUMLYSYL_PEA_1_T22 (SEQ ID | 1754 | 1862 |
| NO: 321) | | |

Segment cluster HUMLYSYL_PEA_{—}1_node_{—}52 (SEQ ID NO:358) according to the present invention is supported by 114 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_T8 (SEQ ID NO:317), HUMLYSYL_PEA_{—}1_T9 (SEQ ID NO:318), HUMLYSYL_PEA_{—}1_T19 (SEQ ID NO:319) and HUMLYSYL_PEA_{—}1_T20 (SEQ ID NO:320). Table 69 below describes the starting and ending position of this segment on each transcript.

TABLE 69
Segment location on transcripts

| | | Segment |
|---|---|---|
| | Segment | ending |
| Transcript name | starting position | position |
| HUMLYSYL_PEA_1_T2 (SEQ ID | 1807 | 1835 |
| NO: 313) | | |
| HUMLYSYL_PEA_1_T4 (SEQ ID | 1895 | 1923 |
| NO: 314) | | |
| HUMLYSYL_PEA_1_T5 (SEQ ID | 1679 | 1707 |
| NO: 315) | | |
| HUMLYSYL_PEA_1_T6 (SEQ ID | 1781 | 1809 |
| NO: 316) | | |
| HUMLYSYL_PEA_1_T8 (SEQ ID | 2010 | 2038 |
| NO: 317) | | |
| HUMLYSYL_PEA_1_T9 (SEQ ID | 1760 | 1788 |
| NO: 318) | | |
| HUMLYSYL_PEA_1_T19 (SEQ ID | 1754 | 1782 |
| NO: 319) | | |
| HUMLYSYL_PEA_1_T20 (SEQ ID | 1754 | 1782 |
| NO: 320) | | |

Segment cluster HUMLYSYL_PEA_{—}1_node_{—}53 (SEQ ID NO:359) according to the present invention is supported by 126 libraries. The number of libraries was determined as previously described. This segment can be found in the following transcript(s): HUMLYSYL_PEA_{—}1_T2 (SEQ ID NO:313), HUMLYSYL_PEA_T4 (SEQ ID NO:314), HUMLYSYL_PEA_{—}1_T5 (SEQ ID NO:315), HUMLYSYL PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319) and HUMLYSYL_PEA_{—}1_T20 (SEQ ID NO:320). Table 70 below describes the starting and ending position of this segment on each transcript.

TABLE 70
Segment location on transcripts

| | | Segment |
|---|---|---|
| | Segment | ending |
| Transcript name | starting position | position |
| HUMLYSYL_PEA_1_T2 (SEQ ID | 1836 | 1911 |
| NO: 313) | | |
| HUMLYSYL_PEA_1_T4 (SEQ ID | 1924 | 1999 |
| NO: 314) | | |
| HUMLYSYL_PEA_1_T5 (SEQ ID | 1708 | 1783 |
| NO: 315) | | |
| HUMLYSYL_PEA_1_T6 (SEQ ID | 1810 | 1885 |
| NO: 316) | | |
| HUMLYSYL_PEA_1_T8 (SEQ ID | 2039 | 2114 |
| NO: 317) | | |
| HUMLYSYL_PEA_1_T9 (SEQ ID | 1789 | 1864 |
| NO: 318) | | |
| HUMLYSYL_PEA_1_T19 (SEQ ID | 1783 | 1858 |
| NO: 319) | | |
| HUMLYSYL_PEA_1_T20 (SEQ ID | 1783 | 1858 |
| NO: 320) | | |

Segment cluster HUMLYSYL_PEA_{—}1_node_{—}56 (SEQ ID NO:360) according to the present invention can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317) and HUMLYSYL_PEA_{—}1_T9 (SEQ ID NO:318). Table 71 below describes the starting and ending position of this segment on each transcript.

TABLE 71
Segment location on transcripts

| | | Segment |
|---|---|---|
| | Segment | ending |
| Transcript name | starting position | position |
| HUMLYSYL_PEA_1_T2 (SEQ ID | 2041 | 2058 |
| NO: 313) | | |
| HUMLYSYL_PEA_1_T4 (SEQ ID | 2129 | 2146 |
| NO: 314) | | |
| HUMLYSYL_PEA_1_T5 (SEQ ID | 1913 | 1930 |
| NO: 315) | | |
| HUMLYSYL_PEA_1_T6 (SEQ ID | 2015 | 2032 |
| NO: 316) | | |
| HUMLYSYL_PEA_1_T8 (SEQ ID | 2244 | 2261 |
| NO: 317) | | |
| HUMLYSYL_PEA_1_T9 (SEQ ID | 1994 | 2011 |
| NO: 318) | | |

Segment cluster HUMLYSYL_PEA_{—}1_node_{—}63 (SEQ ID NO:361) according to the present invention can be found in the following transcript(s): HUMLYSYL PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318) and HUMLYSYL_PEA_{—}1_T24 (SEQ ID NO:322). Table 72 below describes the starting and ending position of this segment on each transcript.

TABLE 72
Segment location on transcripts

| | | Segment |
|---|---|---|
| | Segment | ending |
| Transcript name | starting position | position |
| HUMLYSYL_PEA_1_T2 (SEQ ID | 2623 | 2644 |
| NO: 313) | | |
| HUMLYSYL_PEA_1_T4 (SEQ ID | 2711 | 2732 |
| NO: 314) | | |
| HUMLYSYL_PEA_1_T5 (SEQ ID | 2495 | 2516 |
| NO: 315) | | |
| HUMLYSYL_PEA_1_T6 (SEQ ID | 2597 | 2618 |
| NO: 316) | | |
| HUMLYSYL_PEA_1_T8 (SEQ ID | 2826 | 2847 |
| NO: 317) | | |
| HUMLYSYL_PEA_1_T9 (SEQ ID | 2576 | 2597 |
| NO: 318) | | |
| HUMLYSYL_PEA_1_T24 (SEQ ID | 544 | 565 |
| NO: 322) | | |

Segment cluster HUMLYSYL_PEA_{—}1_node_{—}64 (SEQ ID NO:362) according to the present invention is supported by 208 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318) and HUMLYSYL_PEA_{—}1_T24 (SEQ ID NO:322). Table 73 below describes the starting and ending position of this segment on each transcript.

TABLE 73
Segment location on transcripts

| | | Segment |
|---|---|---|
| | Segment | ending |
| Transcript name | starting position | position |
| HUMLYSYL_PEA_1_T2 (SEQ ID | 2645 | 2674 |
| NO: 313) | | |
| HUMLYSYL_PEA_1_T4 (SEQ ID | 2733 | 2762 |
| NO: 314) | | |
| HUMLYSYL_PEA_1_T5 (SEQ ID | 2517 | 2546 |
| NO: 315) | | |
| HUMLYSYL_PEA_1_T6 (SEQ ID | 2619 | 2648 |
| NO: 316) | | |
| HUMLYSYL_PEA_1_T8 (SEQ ID | 2848 | 2877 |
| NO: 317) | | |
| HUMLYSYL_PEA_1_T9 (SEQ ID | 2598 | 2627 |
| NO: 318) | | |
| HUMLYSYL_PEA_1_T24 (SEQ ID | 566 | 595 |
| NO: 322) | | |

Segment cluster HUMLYSYL_PEA_{—}1_node_{—}66 (SEQ ID NO:363) according to the present invention can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL PEA_{—}1_T19 (SEQ ID NO:319) and HUMLYSYL_PEA_{—}1_T24 (SEQ ID NO:322). Table 74 below describes the starting and ending position of this segment on each transcript.

TABLE 74
Segment location on transcripts

| | | Segment |
|---|---|---|
| | Segment | ending |
| Transcript name | starting position | position |
| HUMLYSYL_PEA_1_T2 (SEQ ID | 2815 | 2821 |
| NO: 313) | | |
| HUMLYSYL_PEA_1_T4 (SEQ ID | 2903 | 2909 |
| NO: 314) | | |
| HUMLYSYL_PEA_1_T5 (SEQ ID | 2687 | 2693 |
| NO: 315) | | |
| HUMLYSYL_PEA_1_T6 (SEQ ID | 2789 | 2795 |
| NO: 316) | | |
| HUMLYSYL_PEA_1_T8 (SEQ ID | 3018 | 3024 |
| NO: 317) | | |
| HUMLYSYL_PEA_1_T9 (SEQ ID | 2768 | 2774 |
| NO: 318) | | |
| HUMLYSYL_PEA_1_T19 (SEQ ID | 1859 | 1865 |
| NO: 319) | | |
| HUMLYSYL_PEA_1_T24 (SEQ ID | 736 | 742 |
| NO: 322) | | |

Segment cluster HUMLYSYL_PEA_{—}1_node_{—}67 (SEQ ID NO:364) according to the present invention is supported by 198 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319) and HUMLYSYL_PEA_{—}1_T24 (SEQ ID NO:322). Table 75 below describes the starting and ending position of this segment on each transcript.

TABLE 75
Segment location on transcripts

| | | Segment |
|---|---|---|
| | Segment | ending |
| Transcript name | starting position | position |
| HUMLYSYL_PEA_1_T2 (SEQ ID | 2822 | 2854 |
| NO: 313) | | |
| HUMLYSYL_PEA_1_T4 (SEQ ID | 2910 | 2942 |
| NO: 314) | | |
| HUMLYSYL_PEA_1_T5 (SEQ ID | 2694 | 2726 |
| NO: 315) | | |
| HUMLYSYL_PEA_1_T6 (SEQ ID | 2796 | 2828 |
| NO: 316) | | |
| HUMLYSYL_PEA_1_T8 (SEQ ID | 3025 | 3057 |
| NO: 317) | | |
| HUMLYSYL_PEA_1_T9 (SEQ ID | 2775 | 2807 |
| NO: 318) | | |
| HUMLYSYL_PEA_1_T19 (SEQ ID | 1866 | 1898 |
| NO: 319) | | |
| HUMLYSYL_PEA_1_T24 (SEQ ID | 743 | 775 |
| NO: 322) | | |

Segment cluster HUMLYSYL_PEA_{—}1_node_{—}68 (SEQ ID NO:365) according to the present invention is supported by 187 libraries. The number of libraries was determined as previously described. This segment can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319) and HUMLYSYL PEA_{—}1_T24 (SEQ ID NO:322). Table 76 below describes the starting and ending position of this segment on each transcript.

TABLE 76
Segment location on transcripts

| | | Segment |
|---|---|---|
| | Segment | ending |
| Transcript name | starting position | position |
| HUMLYSYL_PEA_1_T2 (SEQ ID | 2855 | 2881 |
| NO: 313) | | |
| HUMLYSYL_PEA_1_T4 (SEQ ID | 2943 | 2969 |
| NO: 314) | | |
| HUMLYSYL_PEA_1_T5 (SEQ ID | 2727 | 2753 |
| NO: 315) | | |
| HUMLYSYL_PEA_1_T6 (SEQ ID | 2829 | 2855 |
| NO: 316) | | |
| HUMLYSYL_PEA_1_T8 (SEQ ID | 3058 | 3084 |
| NO: 317) | | |
| HUMLYSYL_PEA_1_T9 (SEQ ID | 2808 | 2834 |
| NO: 318) | | |
| HUMLYSYL_PEA_1_T19 (SEQ ID | 1899 | 1925 |
| NO: 319) | | |
| HUMLYSYL_PEA_1_T24 (SEQ ID | 776 | 802 |
| NO: 322) | | |

Segment cluster HUMLYSYL_PEA_{—}1_node_{—}70 (SEQ ID NO:366) according to the present invention can be found in the following transcript(s): HUMLYSYL_PEA_{—}1_T2 (SEQ ID NO:313), HUMLYSYL_PEA_{—}1_T4 (SEQ ID NO:314), HUMLYSYL_PEA_{—}1_T5 (SEQ ID NO:315), HUMLYSYL_PEA_{—}1_T6 (SEQ ID NO:316), HUMLYSYL_PEA_{—}1_T8 (SEQ ID NO:317), HUMLYSYL_PEA_{—}1_T9 (SEQ ID NO:318), HUMLYSYL_PEA_{—}1_T19 (SEQ ID NO:319), HUMLYSYL_PEA_{—}1_T20 (SEQ ID NO:320) and HUMLYSYL_PEA_{—}1_T24 (SEQ ID NO:322). Table 77 below describes the starting and ending position of this segment on each transcript.

TABLE 77
Segment location on transcripts

| | | Segment |
|---|---|---|
| | Segment | ending |
| Transcript name | starting position | position |
| HUMLYSYL_PEA_1_T2 (SEQ ID | 2882 | 2894 |
| NO: 313) | | |
| HUMLYSYL_PEA_1_T4 (SEQ ID | 2970 | 2982 |
| NO: 314) | | |
| HUMLYSYL_PEA_1_T5 (SEQ ID | 2754 | 2766 |
| NO: 315) | | |
| HUMLYSYL_PEA_1_T6 (SEQ ID | 2856 | 2868 |
| NO: 316) | | |
| HUMLYSYL_PEA_1_T8 (SEQ ID | 3085 | 3097 |
| NO: 317) | | |
| HUMLYSYL_PEA_1_T9 (SEQ ID | 2835 | 2847 |
| NO: 318) | | |
| HUMLYSYL_PEA_1_T19 (SEQ ID | 1926 | 1938 |
| NO: 319) | | |
| HUMLYSYL_PEA_1_T20 (SEQ ID | 1859 | 1871 |
| NO: 320) | | |
| HUMLYSYL_PEA_1_T24 (SEQ ID | 803 | 815 |
| NO: 322) | | |

Variant protein alignment to the previously known protein:

Sequence name: PLO1_HUMAN_V1 (SEQ ID NO:368)

| Sequence documentation: | | | |
|---|---|---|---|
| Alignment of: HUMLYSYL_PEA_1_P2 (SEQ ID NO:369) × | | | |
| PLO1_HUMAN_V1 (SEQ ID NO:368) . . | | | |
| Alignment segment 1/1: | | | |
| Quality: | 4794.00 | Escore: | 0 |
| Matching length: | 490 | Total length: | 490 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALDEVVLKFEMGHVRARNLAYDTLPVLIHGNGPTKLQL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DHRCRIFQNLDGALDEVVLKFEMGHVRARNLAYDTLPVLIHGNGPTKLQL | 250 |
| | . . . . . | |
| 251 | NYLGNYIPRFWTFETGCTVCDEGLRSLKGIGDEALPTVLVGVFIEQPTPF | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | NYLGNYIPRFWTFETGCTVCDEGLRSLKGIGDEALPTVLVGVFIEQPTPF | 300 |
| | . . . . . | |
| 301 | VSLFFQRLLRLHYPQKHMRLFIHNHEQHHKAQVEEFLAQHGSEYQSVKLV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | VSLFFQRLLRLHYPQKHMRLFIHNHEQHHKAQVEEFLAQHGSEYQSVKLV | 350 |
| | . . . . . | |
| 351 | GPEVRMANADARNMGADLCRQDRSCTYYFSVDADVALTEPNSLRLLIQQN | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPEVRMANADARNMGADLCRQDRSCTYYFSVDADVALTEPNSLRLLIQQN | 400 |
| | . . . . . | |
| 401 | KNVIAPLMTRHGRLWSNFWGALSADGYYARSEDYVDIVQGRRVGVWNVPY | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | KNVIAPLMTRHGRLWSNFWGALSADGYYARSEDYVDIVQGRRVGVWNVPY | 450 |
| | . . . . | |
| 451 | ISNIYLIKGSALRGELQSSDLFHHSKLDPDMAFCANIRQQ | 490 |
| | \|\|\|\|\|\|\|\|\|\|\|\|\|\|\|\|\|\|\|\|\|\|\|\|\|\|\|\|\|\|\|\|\|\|\|\|\|\|\|\| | |
| 451 | ISNIYLIKGSALRGELQSSDLFHHSKLDPDMAFCANIRQQ | 490 |

| Sequence name: PLO1_HUMAN_V1 (SEQ ID NO:368) | | | |
|---|---|---|---|
| Sequence documentation: | | | |
| Alignment of: HUMLYSYL_PEA_1_P4 (SEQ ID NO:370) × | | | |
| PLO1_HUMAN_V1 (SEQ ID NO:368) . . | | | |
| Alignment segment 1/1: | | | |
| Quality: | 7109.00 | Escore: | 0 |
| Matching length: | 727 | Total length: | 774 |
| Matching Percent | 100.00 | Matching Percent | 100.00 |
| Similarity: | | Identity: | |
| Total Percent | 93.93 | Total Percent | 93.93 |
| Similarity: | | Identity: | |
| Gaps: | 1 | | |

| Alignment: | | |
|---|---|---|
| | . . . . . | |
| 1 | MRPLLLLALLGWLLLAEAKGDAKPEAPCCQEGLRAGGSGSLHLGRDFTVL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPE......................... | 25 |
| | . . . . . | |
| 51 | AGARGSPSPSVSSIPRFWIPGSDNLLVLTVATKETEGFRRFKRSAQFFNY | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 26 | ......................DNLLVLTVATKETEGFRRFKRSAQFFNY | 53 |
| | . . . . . | |
| 101 | KIQALGLGEDWNVEKGTSAGGGQKVRLLKKALEKHADKEDLVILFADSYD | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 54 | KIQALGLGEDWNVEKGTSAGGGQKVRLLKKALEKHADKEDLVILFADSYD | 103 |
| | . . . . . | |
| 151 | VLFASGPRELLKKFRQARSQVVFSAEELIYPDRRLETKYPVVSDGKRFLG | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 104 | VLFASGFRELLKKFRQARSQVVFSAEELIYPDRRLETKYPVVSDGKRFLG | 153 |
| | . . . . . | |
| 201 | SGGFIGYAPNLSKLVAEWEGQDSDSDQLFYTKIFLDPEKREQINITLDHR | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 154 | SGGFIGYAPNLSKLVAEWEGQDSDSDQLFYTKIFLDPEKREQINITLDHR | 203 |
| | . . . . . | |
| 251 | CRIFQNLDGALDEVVLKFEMGHVRARNLAYDTLPVLIHGNGPTKLQLNYL | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 204 | CRIFQNLDGALDEVVLKFEMGHVRARNLAYDTLPVLIHGNGPTKLQLNYL | 253 |
| | . . . . . | |
| 301 | GNYIPRFWTFETGCTVCDEGLRSLKGIGDEALPTVLVGVFIEQPTPFVSL | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 254 | GNYIPRFWTFETGCTVCDEGLRSLKGIGDEALPTVLVGVFIEQPTPFVSL | 303 |
| | . . . . . | |
| 351 | FFQRLLRLHYPQKHMRLFIHNHEQHHKAQVEEFLAQHGSEYQSVKLVGPE | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 304 | FFQRLLRLHYPQKHMRLFIHNHEQHHKAQVEEFLAQHGSEYQSVKLVGPE | 353 |
| | . . . . . | |
| 401 | VRMANADARNMGADLCRQDRSCTYYFSVDADVALTEPNSLRLLIQQNKNV | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 354 | VRMANADARNMGADLCRQDRSCTYYFSVDADVALTEPNSLRLLIQQNKNV | 403 |
| | . . . . . | |
| 451 | IAPLMTRHGRLWSNFWGALSADGYYARSEDYVDIVQGRRVGVWNVPYISN | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 404 | IAPLMTRHGRLWSNFWGALSADGYYARSEDYVDIVQGRRVGVWNVPYISN | 453 |
| | . . . . . | |
| 501 | IYLIKGSALRGELQSSDLFHHSKLDPDMAFCANIRQQDVFMFLTNRHTLG | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 454 | IYLIKGSALRGELQSSDLFHHSKLDPDMAFCANIRQQDVFMFLTNRHTLG | 503 |
| | . . . . . | |
| 551 | HLLSLDSYRTTHLHNDLWEVFSNPEDWKEKYIHQNYTKALAGKLVETPCP | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 504 | HLLSLDSYRTTHLHNDLWEVFSNPEDWKEKYIHQNYTKALAGKLVETPCP | 553 |
| | . . . . . | |
| 601 | DVYWFPIFTEVACDELVEEMEHFGQWSLGNNKDNRIQGGYENVPTIDIHM | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 554 | DVYWFPIFTEVACDELVEEMEHFGQWSLGNNKDNRIQGGYENVPTIDIHM | 603 |
| | . . . . . | |
| 651 | NQIGFEREWHKFLLEYIAPMTEKLYPGYYTRAQFDLAFVVRYKPDEQPSL | 700 |
| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| 604 | NQIGFEREWHKFLLEYIAPMTEKLYPGYYTRAQFDLAFVVRYKPDEQPSL | 653 |
| | . . . . . | |
| 701 | MPHHDASTFTINIALNRVGVDYEGGGCRFLRYNCSIRAPRKGWTLMHPGR | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 654 | MPHHDASTFTINIALNRVGVDYEGGGCRFLRYNCSIRAPRKGWTLMHPGR | 703 |
| | . . | |
| 751 | LTHYHEGLPTTRGTRYIAVSFVDP | 774 |
| | \|\|\|\|\|\|\|\|\|\|\|\|\|\|\|\|\|\|\|\|\|\|\|\| | |
| 704 | LTHYHEGLPTTRGTRYIAVSFVDP | 727 |

| Sequence name: PLO1_HUMAN_V1 (SEQ ID NO:368) | | | |
|---|---|---|---|
| Sequence documentation: | | | |
| Alignment of: HUMLYSYL_PEA_1_P5 (SEQ ID NO:371) × | | | |
| PLO1_HUMAN_V1 (SEQ ID NO:368) . . | | | |
| Alignment segment 1/1: | | | |
| Quality: | 6869.00 | Escore: | 0 |
| Matching length: | 702 | Total length: | 727 |
| Matching Percent Similarity: | 100.00 | Matching Percent Identity: | 100.00 |
| Total Percent Similarity: | 96.56 | Total Percent Identity: | 96.56 |
| Gaps: | 1 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALDEVVLKFEMGHVRARNLAYDTLPVLIHGNGPTKLQL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DHRCRIFQNLDGALDEVVLKFEMGHVRARNLAYDTLPVLIHGNGFTKLQL | 250 |
| | . . . . . | |
| 251 | NYLGNYIPRFWTFETGCTVCDEGLRSLKGIG................... | 281 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | NYLGNYIPRFWTFETGCTVCDEGLRSLKGIGDEALPTVLVGVFIEQPTPF | 300 |
| | . . . . . | |
| 282 | ......RLLRLHYPQKHMRLFIHNHEQHHKAQVEEFLAQHGSEYQSVKLV | 325 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | VSLFFQRLLRLHYPQKHMRLFIHNHEQHHKAQVEEFLAQHGSEYQSVKLV | 350 |
| | . . . . . | |
| 326 | GPEVRMANADARNMGADLCRQDRSCTYYFSVDADVALTEPNSLRLLIQQN | 375 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPEVRMANADARNMGADLCRQDRSCTYYFSVDADVALTEPNSLRLLIQQN | 400 |
| | . . . . . | |
| 376 | KNVIAPLMTRHGRLWSNFWGALSADGYYARSEDYVDIVQGRRVGVWNVPY | 425 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | KNVIAPLMTRHGRLWSNFWGALSADGYYARSEDYVDIVQGRRVGVWNVPY | 450 |
| | . . . . . | |
| 426 | ISNIYLIKGSALRGELQSSDLFHHSKLDPDMAFCANIRQQDVFMFLTNRH | 475 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | ISNIYLIKGSALRGELQSSDLFHHSKLDPDMAFCANIRQQDVFMFLTNRH | 500 |
| | . . . . . | |
| 476 | TLGHLLSLDSYRTTHLHNDLWEVFSNPEDWKEKYIHQNYTKALAGKLVET | 525 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | TLGHLLSLDSYRTTHLHNDLWEVFSNPEDWKEKYIHQNYTKALAGKLVET | 550 |
| | . . . . . | |
| 526 | PCPDVYWFPIFTEVACDELVEEMEHFGQWSLGNNKDNRIQGGYENVPTID | 575 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | PCPDVYWFPIFTEVACDELVEEMEHFGQWSLGNNKDNRIQGGYENVPTID | 600 |
| | . . . . . | |
| 576 | IHMNQIGFEREWHKFLLEYIAPMTEKLYPGYYTRAQFDLAFVVRYKPDEQ | 625 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | IHMNQIGFEREWHKFLLEYIAPMTEKLYPGYYTRAQFDLAFVVRYKPDEQ | 650 |
| | . . . . . | |
| 626 | PSLMPHHDASTFTINIALNRVGVDYEGGGCRFLRYNCSIRAPRKGWTLMH | 675 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | PSLMPHHDASTFTINIALNRVGVDYEGGGCRFLRYNCSIRAPRKGWTLMH | 700 |
| | . . | |
| 676 | PGRLTHYHEGLPTTRGTRYIAVSFVDP | 702 |
| | \|\|\|\|\|\|\|\|\|\|\|\|\|\|\|\|\|\|\|\|\|\|\|\|\|\|\| | |
| 701 | PGRLTHYHEGLPTTRGTRYIAVSFVDP | 727 |

| Sequence name: PLO1_HUMAN_V1 (SEQ ID NO:368) | | | |
|---|---|---|---|
| Sequence documentation: | | | |
| Alignment of: HUMLYSYL_PEA_1_P6 (SEQ ID NO:372) × | | | |
| PLO1_HUMAN_V1 (SEQ ID NO:368) . . | | | |
| Alignment segment 1/1: | | | |
| Quality: | 7109.00 | Escore: | 0 |
| Matching length: | 727 | Total length: | 736 |
| Matching Percent | 100.00 | Matching Percent | 100.00 |
| Similarity: | | Identity: | |
| Total Percent | 98.78 | Total Percent | 98.78 |
| Similarity: | | Identity: | |
| Gaps: | 1 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQPVLRGVSLQALGLGEDWNVEKGTSAGGGQKVRLLKKALEKHADK | 100 |
| | \|\|\|\|\| \|\|\|\|\|\|\|\|\|\|\|\|\|\|\|\|\|\|\|\|\|\|\|\|\|\|\|\|\|\|\|\|\|\|\|\| | |
| 51 | FNYKI.........QALGLGEDWNVEKGTSAGGGQKVRLLKKALEKHADK | 91 |
| | . . . . . | |
| 101 | EDLVILFADSYDVLFASGPRELLKKFRQARSQVVFSAEELIYPDRRLETK | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 92 | EDLVILFADSYDVLFASGPRELLKKFRQARSQVVFSAEELIYPDRRLETK | 141 |
| | . . . . . | |
| 151 | YPVVSDGKRFLGSGGFIGYAPNLSKLVAEWEGQDSDSDQLFYTKIFLDPE | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 142 | YPVVSDGKRFLGSGGFIGYAPNLSKLVAEWEGQDSDSDQLFYTKIFLDPE | 191 |
| | . . . . . | |
| 201 | KREQINITLDHRCRIFQNLDGALDEVVLKFEMGHVRARNLAYDTLFVLIH | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 192 | KREQINITLDHRCRIFQNLDGALDEVVLKFEMGHVRARNLAYDTLPVLIH | 241 |
| | . . . . . | |
| 251 | GNGPTKLQLNYLGNYIPRFWTFETGCTVCDEGLRSLKGIGDEALPTVLVG | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 242 | GNGPTKLQLNYLGNYIPRFWTFETGCTVCDEGLRSLKGIGDEALPTVLVG | 291 |
| | . . . . . | |
| 301 | VFIEQPTPFVSLFFQRLLRLHYPQKHMRLFIHNHEQHHKAQVEEFLAQHG | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 292 | VFIEQPTPFVSLFFQRLLRLHYPQKHMRLFIHNHEQHHKAQVEEFLAQHG | 341 |
| | . . . . . | |
| 351 | SEYQSVKLVGPEVRMANADARNMGADLCRQDRSCTYYFSVDADVALTEPN | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 342 | SEYQSVKLVGPEVRMANADARNMGADLCRQDRSCTYYFSVDADVALTEPN | 391 |
| | . . . . . | |
| 401 | SLRLLIQQNKNVIAPLMTRHGRLWSNFWGALSADGYYARSEDYVDIVQGR | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 392 | SLRLLIQQNKNVIAPLMTRHGRLWSNFWGALSADGYYARSEDYVDIVQGR | 441 |
| | . . . . . | |
| 451 | RVGVWNVPYISNIYLIKGSALRGELQSSDLFHHSKLDPDMAFCANIRQQD | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 442 | RVGVWNVPYISNIYLIKGSALRGELQSSDLFHHSKLDPDMAFCANIRQQD | 491 |
| | . . . . . | |
| 501 | VFMFLTNRHTLGHLLSLDSYRTTHLHNDLWEVFSNPEDWKEKYIHQNYTK | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 492 | VFMFLTNRHTLGHLLSLDSYRTTHLHNDLWEVFSNFEDWKEKYIHQNYTK | 541 |
| | . . . . . | |
| 551 | ALAGKLVETPCPDVYWFPIFTEVACDELVEEMEHFGQWSLGNNKDNRIQG | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 542 | ALAGKLVETPCPDVYWFPIFTEVACDELVEEMEHFGQWSLGNNKDNRIQG | 591 |
| | . . . . . | |
| 601 | GYENVPTIDIHMNQIGFEREWHKFLLEYIAPMTEKLYPGYYTRAQFDLAF | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 592 | GYENVPTIDIHMNQIGFEREWHKFLLEYIAPMTEKLYPGYYTRAQFDLAF | 641 |
| | . . . . . | |
| 651 | VVRYKPDEQPSLMPHHDASTFTINIALNRVGVDYEGGGCRFLRYNCSIRA | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 642 | VVRYKPDEQPSLMPHHDASTFTINIALNRVGVDYEGGGCRFLRYNCSIRA | 691 |
| | . . . | |
| 701 | PRKGWTLMHPGRLTHYHEGLPTTRGTRYIAVSFVDP | 736 |
| | \|\|\|\|\|\|\|\|\|\|\|\|\|\|\|\|\|\|\|\|\|\|\|\|\|\|\|\|\|\|\|\|\|\|\|\| | |
| 692 | PRKGWTLMHPGRLTHYHEGLPTTRGTRYIAVSFVDP | 727 |

| Sequence name: PLO1_HUMAN_V1 (SEQ ID NO:368) | | | |
|---|---|---|---|
| Sequence documentation: | | | |
| Alignment of: HUMLYSYL_PEA_1_P7 (SEQ ID NO:373) × | | | |
| PLO1_HUMAN_V1 (SEQ ID NO:368) . . | | | |
| Alignment segment 1/1: | | | |
| Quality: | 6697.00 | Escore: | 0 |
| Matching length: | 698 | Total length: | 758 |
| Matching Percent | 99.71 | Matching Percent | 99.71 |
| Similarity: | | Identity: | |
| Total Percent | 91.82 | Total Percent | 91.82 |
| Similarity: | | Identity: | |
| Gaps: | 2 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALVSPWGQGHLPGACYELTASVLTSELSVMPSFPAVV. | 249 |
| | \|\|\|\|\|\|\|\|\|\|\|\|\|\| \|\| | |
| 201 | DHRCRIFQNLDGAL...............................DEVVL | 219 |
| | . . . . . | |
| 250 | ............................LQLNYLGNYIPRFWTFETGCTV | 271 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 220 | KFEMGHVRARNLAYDTLPVLIHGNGPTKLQLNYLGNYIPRFWTFETGCTV | 269 |
| | . . . . . | |
| 272 | CDEGLRSLKGIGDEALPTVLVGVFIEQPTPFVSLFFQRLLRLHYPQKHMR | 321 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 270 | CDEGLRSLKGIGDEALPTVLVGVFIEQPTPFVSLFFQRLLRLHYFQKHMR | 319 |
| | . . . . . | |
| 322 | LFIHNHEQHHKAQVEEFLAQHGSEYQSVKLVGPEVRMANADARNMGADLC | 371 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 320 | LFIHNHEQHHKAQVEEFLAQHGSEYQSVKLVGPEVRMANADARNMGADLC | 369 |
| | . . . . . | |
| 372 | RQDRSCTYYFSVDADVALTEPNSLRLLIQQNKNVIAPLMTRHGRLWSNFW | 421 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 370 | RQDRSCTYYFSVDADVALTEPNSLRLLIQQNKNVIAPLMTRHGRLWSNFW | 419 |
| | . . . . . | |
| 422 | GALSADGYYARSEDYVDIVQGRRVGVWNVPYISNIYLIKGSALRGELQSS | 471 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 420 | GALSADGYYARSEDYVDIVQGRRVGVWNVPYISNIYLIKGSALRGELQSS | 469 |
| | . . . . . | |
| 472 | DLFHHSKLDPDMAFCANIRQQDVFNFLTNRHTLGHLLSLDSYRTTHLHND | 521 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 470 | DLFHHSKLDPDMAFCANIRQQDVFMFLTNRHTLGHLLSLDSYRTTHLHND | 519 |
| | . . . . . | |
| 522 | LWEVFSNPEDWKEKYIHQNYTKALAGKLVETPCPDVYWFPIFTEVACDEL | 571 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 520 | LWEVFSNPEDWKEKYIHQNYTKALAGKLVETPCPDVYWFPIFTEVACDEL | 569 |
| | . . . . . | |
| 572 | VEEMEHFGQWSLGNNKDNRIQGGYENVPTIDIHMNQIGFEREWHKFLLEY | 621 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 570 | VEEMEHFGQWSLGNNKDNRIQGGYENVPTIDIHMNQIGFEREWHKFLLEY | 619 |
| | . . . . . | |
| 622 | IAPMTEKLYPGYYTRAQFDLAFVVRYKPDEQPSLMPHHDASTFTINIALN | 671 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 620 | IAPMTEKLYPGYYTRAQFDLAFVVRYKPDEQPSLMPHHDASTFTINIALN | 669 |
| | . . . . . | |
| 672 | RVGVDYEGGGCRFLRYNCSIRAPRKGWTLMHPGRLTHYHEGLPTTRGTRY | 721 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 670 | RVGVDYEGGGCRFLRYNCSIRAPRKGWTLMHPGRLTHYHEGLPTTRGTRY | 719 |
| 722 | IAVSFVDP | 729 |
| | \|\|\|\|\|\|\|\| | |
| 720 | IAVSFVDP | 727 |

| Sequence name: PLO1_HUMAN_V1 (SEQ ID NO:368) | | | |
|---|---|---|---|
| Sequence documentation: | | | |
| Alignment of: HUMLYSYL_PEA_1 P13 (SEQ ID NO:374) × | | | |
| PLO1_HUMAN_V1 (SEQ ID NO:368) . . | | | |
| Alignment segment 1/1: | | | |
| Quality: | 5773.00 | Escore: | 0 |
| Matching length: | 585 | Total length: | 585 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALDEVVLKFEMGHVRARNLAYDTLPVLIHGNGPTKLQL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DHRCRIFQNLDGALDEVVLKFEMGHVRARNLAYDTLPVLIHGNGPTELQL | 250 |
| | . . . . . | |
| 251 | NYLGNYIPRFWTFETGCTVCDEGLRSLKGIGDEALPTVLVGVFIEQPTPF | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | NYLGNYIPRFWTFETGCTVCDEGLRSLKGIGDEALPTVLVGVFIEQPTPF | 300 |
| | . . . . . | |
| 301 | VSLFFQRLLRLHYPQKHMRLFIHNHEQHHKAQVEEFLAQHGSEYQSVKLV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | VSLFFQRLLRLHYPQKHMRLFIHNHEQHHKAQVEEFLAQHGSEYQSVKLV | 350 |
| | . . . . . | |
| 351 | GPEVRMANADARNMGADLCRQDRSCTYYFSVDADVALTEPNSLRLLIQQN | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPEVRMANADARNMGADLCRQDRSCTYYFSVDADVALTEPNSLRLLIQQN | 400 |
| | . . . . . | |
| 401 | KNVIAPLMTRHGRLWSNFWGALSADGYYARSEDYVDIVQGRRVGVWNVPY | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | KNVIAPLMTRHGRLWSNFWGALSADGYYARSEDYVDIVQGRRVGVWNVPY | 450 |
| | . . . . . | |
| 451 | ISNIYLIKGSALRGELQSSDLFHHSKLDPDMAFCANIRQQDVFMFLTNRH | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | ISNIYLIKGSALRGELQSSDLFHHSKLDPDMAFCANIRQQDVFMFLTNRH | 500 |
| | . . . . . | |
| 501 | TLGHLLSLDSYRTTHLHNDLWEVFSNPEDWKEKYIHQNYTKALAGKLVET | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | TLGHLLSLDSYRTTHLHNDLWEVFSNPEDWKEKYIHQNYTKALAGKLVET | 550 |
| | . . . | |
| 551 | PCPDVYWFPIFTEVACDELVEEMEHFGQWSLGNNK | 585 |
| | \|\|\|\|\|\|\|\|\|\|\|\|\|\|\|\|\|\|\|\|\|\|\|\|\|\|\|\|\|\|\|\|\|\|\| | |
| 551 | PCPDVYWFPIFTEVACDELVEEMEHFGQWSLGNNK | 585 |

Sequence name: PLO1_HUMAN_V1 (SEQ ID NO:368)
Sequence documentation:
Alignment of: HUMLYSYL_PEA_1_P14 (SEQ ID NO:375) ×
PLO1_HUMAN_V1 (SEQ ID NO: 368) . .
Alignment segment 1/1:

| | | | |
|---|---|---|---|
| +TL,Quality: | 5773.00 | Escore: | 0 |
| Matching length: | 585 | Total length: | 585 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALDEVVLKFEMGHVRARNLAYDTLPVLIHGNGPTKLQL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DHRCRIFQNLDGALDEVVLKFEMGHVRARNLAYDTLPVLIHGNGPTKLQL | 250 |
| | . . . . . | |
| 251 | NYLGNYIPRFWTFETGCTVCDEGLRSLKGIGDEALPTVLVGVFIEQPTPF | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | NYLGNYIPRFWTFETGCTVCDEGLRSLKGIGDEALPTVLVGVFIEQPTPF | 300 |
| | . . . . . | |
| 301 | VSLFFQRLLRLHYPQKHMRLFIHNHEQHHKAQVEEFLAQHGSEYQSVKLV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | VSLFFQRLLRLHYPQKHMRLFIHNHEQHHKAQVEEFLAQHGSEYQSVKLV | 350 |
| | . . . . . | |
| 351 | GPEVRMANADARNMGADLCRQDRSCTYYFSVDADVALTEPNSLRLLIQQN | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPEVRMANADARNMGADLCRQDRSCTYYFSVDADVALTEPNSLRLLIQQN | 400 |
| | . . . . . | |
| 401 | KNVIAPLMTRHGRLWSNFWGALSADGYYARSEDYVDIVQGRRVGVWNVPY | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | KNVIAPLMTRHGRLWSNFWGALSADGYYARSEDYVDIVQGRRVGVWNVPY | 450 |
| | . . . . . | |
| 451 | ISNIYLIKGSALRGELQSSDLFHHSKLDPDMAFCANIRQQDVFMFLTNRH | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | ISNIYLIKGSALRGELQSSDLFHHSKLDPDMAFCANIRQQDVFNFLTNRH | 500 |
| | . . . . . | |
| 501 | TLGHLLSLDSYRTTHLHNDLWEVFSNPEDWKEKYIHQNYTKALAGKLVET | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | TLGHLLSLDSYRTTHLHNDLWEVFSNPEDWKEKYIHQNYTKALAGKLVET | 550 |
| | . . . | |
| 551 | PCPDVYWFPIFTEVACDELVEEMEHFGQWSLGNNK | 585 |
| | \|\|\|\|\|\|\|\|\|\|\|\|\|\|\|\|\|\|\|\|\|\|\|\|\|\|\|\|\|\|\|\|\|\|\| | |
| 551 | PCPDVYWFPIFTEVACDELVEEMEHFGQWSLGNNK | 585 |

| Sequence name: PLO1_HUMAN_V1 (SEQ ID NO:368) | | | |
|---|---|---|---|
| Sequence documentation: | | | |
| Alignment of: HUMLYSYL_PEA_1_P16 (SEQ ID NO:376) × | | | |
| PLO1_HUMAN_V1 (SEQ ID NO:368) . . | | | |
| Alignment segment 1/1: | | | |
| Quality: | 5400.00 | Escore: | 0 |
| Matching length: | 550 | Total length: | 550 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYFVVSDGKR | 150 |
| | . . . . . | |
| 151 | FLGSGGFIGYAPNLSKLVAEWEGQDSDSDQLFYTKIFLDPEKREQINITL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKREQINITL | 200 |
| | . . . . . | |
| 201 | DHRCRIFQNLDGALDEVVLKFEMGHVRARNLAYDTLPVLIHGNGPTKLQL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DHRCRIFQNLDGALDEVVLKFEMGHVRARNLAYDTLPVLIHGNGPTKLQL | 250 |
| | . . . . . | |
| 251 | NYLGNYIPRFWTFETGCTVCDEGLRSLKGIGDEALPTVLVGVFIEQPTPF | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | NYLGNYIPRFWTFETGCTVCDEGLRSLKGIGDEALPTVLVGVFIEQPTPF | 300 |
| | . . . . . | |
| 301 | VSLFFQRLLRLHYPQKHMRLFIHNHEQHHKAQVEEFLAQHGSEYQSVKLV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | VSLFFQRLLRLHYPQKHMRLFIHNHEQHHKAQVEEFLAQHGSEYQSVKLV | 350 |
| | . . . . . | |
| 351 | GPEVRMANADARNMGADLCRQDRSCTYYFSVDADVALTEPNSLRLLIQQN | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | GPEVRMANADARNMGADLCRQDRSCTYYFSVDADVALTEPNSLRLLIQQN | 400 |
| | . . . . . | |
| 401 | KNVIAPLMTRXGRLWSNFWGALSADGYYARSEDYVDIVQGRRVGVWNVPY | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | KNVIAPLMTRHGRLWSNFWGALSADGYYARSEDYVDIVQGRRVGVWNVPY | 450 |
| | . . . . . | |
| 451 | ISNIYLIKGSALRGELQSSDLFHHSKLDPDMAFCANIRQQDVFMFLTNRH | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | ISNIYLIKGSALRGELQSSDLFHHSKLDPDMAFCANIRQQDVFMFLTNRH | 500 |
| | . . . . . | |
| 501 | TLGHLLSLDSYRTTHLHNDLWEVFSNPEDWKEKYIHQNYTKALAGKLVET | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | TLGHLLSLDSYRTTHLHNDLWEVFSNPEDWKEKYIHQNYTKALAGKLVET | 550 |

| Sequence name: PLO1_HUMAN_V1 (SEQ ID NO:368) | | | |
|---|---|---|---|
| Sequence documentation: | | | |
| Alignment of: HUMLYSYL_PEA_1_P24 (SEQ ID NO:378) × | | | |
| PLO1_HUMAN_V1 (SEQ ID NO:368) . . | | | |
| Alignment segment 1/1: | | | |
| Quality: | 1850.00 | Escore: | 0 |
| Matching length: | 193 | Total length: | 193 |
| Matching Percent | 100.00 | Matching Percent | 100.00 |
| Similarity: | | Identity: | |
| Total Percent | 100.00 | Total Percent | 100.00 |
| Similarity: | | Identity: | |
| Gaps: | 0 | | |

| Alignment: | | |
|---|---|---|
| | . . . . . | |
| 1 | MRPLLLLALLGWLLLAEAKGDAKPEDNLLVLTVATKETEGFRRFKRSAQF | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MRPLLLLALLGWLLLAEAKGDAKPEDNLLVLTVATKETEGFRRFKRSAQF | 50 |
| | . . . . . | |
| 51 | FNYKIQALGLGEDWNVEKGTSAGGGQKVRLLKKALEKHADKEDLVILFAD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | FNYKIQALGLGEDWNVEKGTSAGGGQKVRLLKKALEKHADKEDLVILFAD | 100 |
| | . . . . . | |
| 101 | SYDVLFASGPRELLKKFRQARSQVVFSAEELIYPDRRLETKYPVVSDGKR | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | SYDVLFASGPRELLKKFRQARSQVVFSAEELIYPDRRLETKYPVVSDGKR | 150 |
| | . . . . | |
| 151 | FLGSGGFIGYAPNLSKLVAEWEGQDSDSDQLFYTKIFLDPEKR | 193 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | FLGSGGFIGYAPNLSKLVAEWEGQDSDSDQLFYTKIFLDPEKR | 193 |

### Additional Examples of Endometrial Markers

The present invention also encompasses additional examples of markers that are suitable for use with endometriosis. These markers relate to the chordin-like-2 (CHL2) family of variants that was discovered by the present applicants. These variants are disclosed in PCT Application No. WO 01/34796 and in PCT Application No. IL2004/000735, both of which are hereby incorporated by reference as if fully set forth herein. Preferably, these markers are serum markers but optionally they are immunohistochemistry markers. They are useful for diagnosis with any suitable biological, including but not limited to the examples listed previously.

As previously published by the present applicants (Oren et al, Gene. 2004 Apr. 28; 331:17-31), these variants bind Activin A specifically (and not BMP-2, 4, 6 as other members of the chordin family). By the literature, Activin A is associated with endometriosis. For example, there is evidence for local production and secretion of Activin A in ovarian endometriotic cysts (Reis et al, Fertil Steril. 2001 February; 75(2):367-73; Florio et al, Steroids. 2003 November; 68(10-13):801-7). All of these references are hereby incorporated by reference as if fully set forth herein. A brief description of these sequences is provided below.

Chordin is an abundant glycoprotein, and is a secreted protein of 955 amino acids (aa) with a molecular mass of 120 Kda. It is a key developmental protein that dorsalizes early vertebrate embryonic tissues by binding to ventralizing TGF-beta-like bone morphogenic proteins (BMP) and sequestering them in latent complexes. BMPs participate in a broad spectrum of cellular inducing events involving all three germ layers during metazoan development. Chordin binds to ventral BMP-2 and BMP-4 signals in the extracellular space, blocking the interaction of BMPs with their receptors. Chordin mimics the action of the Spemann organizer and can induce the formation of neural tissue from ectoderm and dorsalization of the ventral mesoderm to form muscle.

During early embryogenesis of vertebrates and invertebrates, antagonism between BMPs and several unrelated proteins is a general mechanism by which the dorso-ventral axis is established. One of these extracellular antagonists is Chordin, which binds with high affinity to certain BMPs, preventing their interaction with their cognate cell surface receptors. Chordin plays a role in dorso-ventral axis formation and induction, as well as in maintenance and differentiation of neural tissues in early vertebrate embryogenesis. The inhibitory activity of Chordin on BMPs is mediated by binding through specific domains named Cysteine-Rich (CR) repeats.

The conservation of each specific CR repeat between Chordin orthologs in different species is higher than that of different CRs within a particular ortholog. The individual CR repeats in Chordin vary in their binding affinity to BMPs, but they function cooperatively in the full-length protein.

Several alternatively spliced transcripts have been reported for the human Chordin gene. These variants were found to be differentially expressed in various tissues, and code for C-truncated isoforms of the Chordin protein that vary in their content of CR repeats and in their biological activity as BMP antagonists.

A New Chordin-like protein (CHL) was recently reported. CHL also binds and inhibits BMP activity. During embryogenesis and organogenesis, Chordin and CHL display distinct spatiotemporal expression patterns. Several splicing variants of mouse and human CHL have been reported which differ primarily in the length and sequence of their C-termini.

CHL has been shown to be secreted and to bind BMPs and other TGFb superfamily members. Expression patterns as well as functional studies in mouse, chicken and xenopus, indicate that it may function as a modulator of BMP signaling during embryonic development.

Recently, another chordin-like protein, which is structurally most homologous to CHL/neuralin/ventroptin, was identified (Development, 2004 January; 131(1):229-40. Epub 2003 Dec. 03.). When injected into Xenopus embryos, RNA of this protein induced a secondary dorso-ventral axis. Recombinant protein interacted directly with BMPs in a competitive manner to prevent binding to the type I BMP receptor ectodomain, and inhibited BMP-dependent induction of alkaline phosphatase in C2Cl2 cells. Thus, this protein behaves as a secreted BMP-binding inhibitor. In situ hybridization revealed that expression of this protein is restricted to chondrocytes of various developing joint cartilage surfaces and connective tissues in reproductive organs. Adult mesenchymal progenitor cells expressed this protein, and its levels decreased during chondrogenic differentiation. Addition of this protein to a chondrogenic culture system reduced cartilage matrix deposition. Consistently, protein transcripts were weakly detected in normal adult joint cartilage. However, its expression was upregulated in middle zone chondrocytes in osteoarthritic joint cartilage (where hypertrophic markers are induced). This protein depressed chondrocyte mineralization when added during the hypertrophic differentiation of cultured hyaline cartilage particles. Thus, this protein may play negative roles in the (re)generation and maturation of articular chondrocytes in the hyaline cartilage of both developing and degenerated joints.

A novel member of the Chordin-like protein family was identified and characterized by the present applicant in human and in mouse (PCT Application No. WO 01/34796, hereby incorporated by reference as if fully set forth herein). This novel protein, named CLH, shows high similarity to the recently reported CHL protein, also named Neuralin-1 or Ventroptin. For the sake of clarity, CLH will be referred to here as CHL2, since it is most closely related to the CHL sequence reported by Nakayama et al.

The high level of homology between CHL2 and CHL is reflected not only in the protein sequence, for example with regard to the number and location of the CR repeats (two adjacent repeats at the N′-terminus, and a third one further downstream), and the absence of other recognizable protein domains, but also in the gene structure, number and size of exons and the spacing of the CR repeats within the exons. Further characterization of CHL2 revealed ubiquitous expression in a variety of tissues and complex alternative splicing, resulting in differentially expressed CHL2 isoforms that differ in their C-termini, the presence of a signal peptide, and the content of their CR repeats.

It has been postulated that Chordin may be expressed by cells of the osteoblast lineage to limit BMP actions in osteoblasts. This may suggest an important function for Chordin as a BMP binding protein since excessive BMP-4 has been implicated in pathogenesis of Fibrodysplasia Ossificans Progressiva (FOP). FOP is a rare genetic disease in which muscles, tendons, ligaments and other connective tissues may ossify into bone. BMPs can cause induction of noggin and Chordin mRNA and protein levels in skeletal cells by transcriptional mechanisms, and these, in turn, prevent the effect of BMPs in osteoblasts in a negative-feedback mechanism. The induction of these proteins by BMPs appears to be a mechanism to limit the BMP effect in bones. Existing therapies which are being investigated for their effectiveness in preventing heterotopic bone formation include inhibitors of BMPs.

The Chordin-like protein 2 (CHL2) variants according to the present invention are useful for diagnosis of endometriosis, as markers. These markers may optionally comprise an isolated nucleic acid molecule comprising the sequence of any one of SEQ ID NO: 379 to SEQ ID NO: 383, fragments of said sequences having at least 20 nucleic acids, or a molecule comprising a sequence having at least 80%, preferably 90%, and most preferably 95% or 98% identity to any one of SEQ ID NO:379 to SEQ ID NO: 383, as well as sequences complementary thereto and/or capable of hybridizing therewith, preferably under moderate to stringent conditions (described above). Optionally and more preferably, a nucleic acid molecule comprising or consisting of a non-coding sequence which is complementary to that of any one of SEQ ID NO: 379 to SEQ ID NO: 383, or complementary to a sequence having at least 80%, preferably 90%, most preferably 95% or 98% identity to said sequences or a fragment of said sequences. The complementary sequence may be a DNA sequence which hybridizes to any one of the sequences of SEQ ID NO: 379 to SEQ ID NO: 383, or hybridizes to a portion of these sequences which includes the “unique” sequences or bridges, and which has a length sufficient to inhibit the transcription of any one of the sequences of SEQ ID NO:379 to SEQ ID NO:383. The complementary sequence may be a DNA sequence which can be transcribed into an mRNA being an antisense of the mRNA transcribed from any one of SEQ ID NO: 379 to SEQ ID NO: 383 amend or into an mRNA which is an antisense to a fragment of the mRNA transcribed from any one of SEQ ID NO: 379 to SEQ ID NO: 383 which has a length sufficient to hybridize with the mRNA transcribed from any one of SEQ ID NO: 379 to SEQ ID NO: 383, so as to inhibit its translation. The complementary sequence may also be the mRNA or the fragment of the mRNA itself.

These markers may optionally comprise a protein or polypeptide comprising or consisting of an amino acid sequence encoded by any of the above nucleic acid sequences, termed herein “CHL2 product”, for example, an amino acid sequence having the sequence in any one of SEQ ID NO: 389 to 393, fragments of the above amino acid sequences having a length of at least 10 amino acids, as well as homologues of the amino acid sequences of any one of SEQ ID NO: 389 to 393 in which one or more of the amino acid residues has been substituted (by conservative or non-conservative substitution) added, deleted, or chemically modified.

Markers according to the present invention may also optionally comprise nucleic acid molecule comprising or consisting of a sequence which encodes the above amino acid sequences (including the fragments and analogs of the amino acid sequences). Due to the degenerative nature of the genetic code, a plurality of alternative nucleic acid sequences, beyond SEQ ID NO: 379 to SEQ ID NO: 383, can code for the amino acid sequence of the invention. Those alternative nucleic acid sequences which code for the same amino acid sequences encoded by the sequences of SEQ ID NO:379 to SEQ ID NO: 383 are also an aspect of the of the present invention.

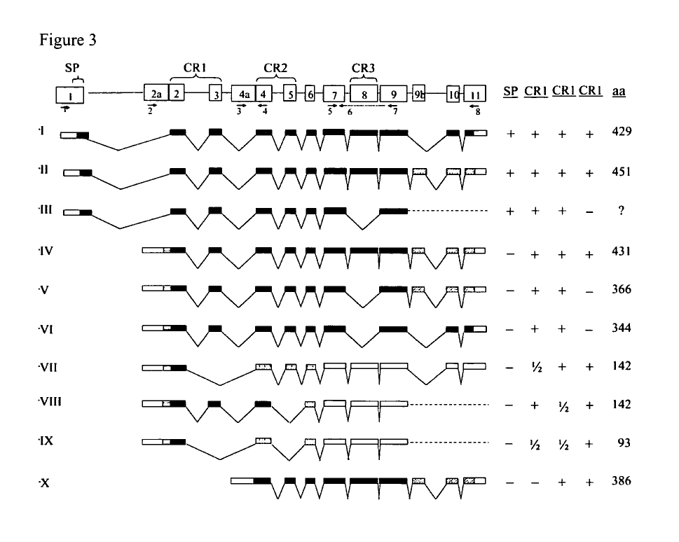

The first variant (SEQ ID NO: 379, termed “Var I” in the figures) lacks exon 9b (FIG. 3), creating a unique sequence (bridge) between exons 9 and 10.

The second variant (SEQ ID NO: 380, termed “Var III” in the figures) is identical to SEQ ID NO: 379 except that it skips exon 8, and ends with exon 9, creating a unique sequence (bridge) between exons 7 and 9.

The third variant (SEQ ID NO: 381, termed “Var VII” in the figures) Starts from exon 2a, skips exon 3 and exon 9b, as described in FIG. 3, creating a unique sequence (bridge) between exon 2 and 4 and another unique sequence (bridge) between 9(a) and 10.

The fourth variant (SEQ ID NO: 382, termed “Var VIII” in the figures) Starts at exon 2a, skips exon 5 and terminates at exon 9, without exons 9b, 10 and 11, creating a unique sequence (bridge) between exons 4 and 6.

The fifth variant (SEQ ID NO: 383, termed “Var IX” in the figures) is identical to SEQ ID NO: 382, but without exon 3, creating a unique sequence (bridge) between exons 2 and 4, and another unique sequence (bridge) between exons 4 and 6.

It should be noted that the amino acid sequences of the above variants (for which nucleic acid sequences are shown in SEQ ID Nos: 379-383) are preferably described as “consisting essentially of” the numbered sequences; for example, the fifth variant preferably is of a nucleic acid sequence having a sequence consisting essentially of the sequence shown in SEQ ID NO:383.

SEQ IDs NO: 389-393 are the amino acid sequences encoded by SEQ IDs NO: 379-383, respectively.

“Primers and Amplicons According to the Present Invention”

SEQ ID NOs: 399-426 are Primers Used for PCR Amplifications:

a. hCHL2:
SEQ ID NO: 399 is referred to in the description below as p1.
SEQ ID NO: 400 is referred to in the description below as p2.
SEQ ID NO: 401 is referred to in the description below as p3.
SEQ ID NO: 402 is referred to in the description below as p4.
SEQ ID NO: 403 is referred to in the description below as p5.
SEQ ID NO: 404 is referred to in the description below as p6.
SEQ ID NO: 405 is referred to in the description below as p7.
SEQ ID NO: 406 is referred to in the description below as p8.
SEQ ID NO: 407 is referred to in the description below as p9.
b. mCHL2:
SEQ ID NO: 408 is referred to in the description below as p1.
SEQ ID NO: 409 is referred to in the description below as p2.
SEQ ID NO: 410 is referred to in the description below as p3.
SEQ ID NO: 411 is referred to in the description below as p4.
SEQ ID NO: 412 is referred to in the description below as p5.
SEQ ID NO: 413 is referred to in the description below as p6.
c. Human Osteocalcin: SEQ ID NOs: 414 and 415.
d. Mouse Osteocalcin: SEQ ID NOs: 416 and 417.
e. Mouse Myogenin: SEQ ID NOs: 418 and 419.
f. ATP synthase 6: SEQ ID NOs: 420 and 421.
g. 26SPSP: SEQ ID NOs: 422 and 423.
h. Mouse GAPDH: SEQ ID NOs: 424 and 425.
SEQ ID NO 426: mouse CHL2 nucleotide sequence
SEQ ID NO 427: mouse CHL2 protein sequence
SEQ ID NO 428: HPRT1-Forward primer
SEQ ID NO 429: HPRT1-Reverse primer
SEQ ID NO 430: HPRT1 amplicon
SEQ ID NO 431: PBGD-Forward primer
SEQ ID NO 432: PBGD-Reverse primer
SEQ ID NO 433: PBGD amplicon
SEQ ID NO 434: SDHA-Forward primer
SEQ ID NO 435: SDHA-Reverse primer
SEQ ID NO 436: SDHA amplicon
SEQ ID NO 437: G6PD-Forward primer
SEQ ID NO 438: G6PD-Reverse primer
SEQ ID NO 439: G6PD amplicon
SEQ ID NO 440: Exon 2a-Forward primer
SEQ ID NO 441: Exon 2a-Reverse primer
SEQ ID NO 442: amplicon exon 2a
SEQ ID NO 443: Ubiquitin-Forward primer
SEQ ID NO 444: Ubiquitin-Reverse primer
SEQ ID NO 445: Ubiquitin Amplicon
SEQ ID NO 446: Exon 4a Forward primer
SEQ ID NO 447: Exon 4a-Reverse primer
SEQ ID NO 448: Exon 4a-amplicon
SEQ ID NO 449: RPL-19-Forward primer
SEQ ID NO 450: RPL-19-Reverse primer
SEQ ID NO 451: RPL-19 amplicon

“CLH2 (Chordin Like Homolog) Sequences”:

All of the sequences described in this section refer to Group II CLH2 sequences.

SEQ ID NO: 384 (described in the figures as “Var II”) Has an accession number of AX140199. Var II contains an additional exon between exons 9 and 10, referred as “9b” in FIG. 3, creating a unique amino acid sequence.

SEQ ID NO: 394 is the amino acid sequence encoded by SEQ ID NO: 384.

SEQ ID NO: 385 (described in the figures as “Var IV”) Has an accession number of AX140202. Var IV starts from a unique exon 2a, as is demonstrated in FIG. 3, and contains an additional exon between exons 9 and 10, referred as “9b” in FIG. 3, creating a unique amino acid sequence. SEQ ID NO: 395 is the amino acid sequence encoded by SEQ ID NO: 385.

SEQ ID NO: 386 (described in the figures as “Var V”) Has an accession number of AX140203. Var V is identical to Var IV, while it skips exon 8, creating a unique sequence (bridge) between exons 7 and 9. SEQ ID NO: 396 is the amino acid sequence encoded by SEQ ID NO: 386.

SEQ ID NO: 387 (described in the figures as “Var VI”) Has an accession number of AX140204. Var VI starts from a unique exon 2a, as is demonstrated in FIG. 3, it skips exon 8, creating a unique sequence (bridge) between exons 7 and 9, and it does not contain exon 9b, creating a unique sequence (bridge) between exons 9 and 10.

SEQ ID NO: 397 is the amino acid sequence encoded by SEQ ID NO: 387.

SEQ ID NO: 388 (described in the figures as “Var X”) Has an accession number of AX140201. Var X starts from a unique exon 4a, as is demonstrated in FIG. 3. SEQ ID NO: 398 is the amino acid sequence encoded by SEQ ID NO: 388.

SEQ ID NOS 452-462 are amino acid sequences corresponding to the nucleic acid sequences shown in SEQ ID NOS 452-462, and so form Group II CLH nucleotide fragments. SEQ ID NOS 463-473 form amino acid sequences corresponding to Group II CLH polypeptides.

SEQ ID NO 474: mouse CHL2, corresponding to genbank accession number: AAH19399.

Thus, Group I sequences include amino acid sequences having at least about 70%, optionally at least about 80%, preferably at least about 85%, more preferably at least about 90% and most preferably at least about 95% homology to any of SEQ ID NOs 389-393; and nucleic acid sequences having at least about 70%, optionally at least about 80%, preferably at least about 85%, more preferably at least about 90% and most preferably at least about 95% homology to any of SEQ ID NOs 379-383.

Group II sequences include amino acid sequences having at least about 70%, optionally at least about 80%, preferably at least about 85%, more preferably at least about 90% and most preferably at least about 95% homology to any of SEQ ID NOs 394-398 or 463-473; and nucleic acid sequences having at least about 70%, optionally at least about 80%, preferably at least about 85%, more preferably at least about 90% and most preferably at least about 95% homology to any of SEQ ID NOs 384-388 or 452-462.

In addition, it should be noted that Group I sequences also have unique bridges. These bridges were noted above for the nucleotide sequences in terms of the exons. They are described below in terms of the amino acid sequences, although it should be noted that optionally a nucleotide sequence could be constructed according to any of the amino acid sequences below and used for any purpose ascribed to a nucleotide sequence as described herein. All the alignments were done against Var II, such that the bridges are described with regard to the amino acid sequence of Var II (SEQ ID NO: 394). The bridge is marked on a portion of the actual sequence below by //, which indicates that a portion of the sequence for that SEQ ID NO (relative to the sequence of Var II) is not present.

(SEQ ID NO 389)

Variant I bridge:
RFALEHEASDLVEIYL WKLVK // GIFHLTQIKKV
RKQDFQKEAQHFRLLA

This bridge is present between amino acid positions 373 (lys) and 374 (gly), and preferably comprises a peptide having a sequence taken from either side of these positions. For example, the peptide could optionally comprise a bridge portion of SEQ ID NO: 389, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KG, having a structure as follows (numbering according to SEQ ID NO:389): a sequence starting from any of amino acid number 373−x to 373; and ending at any of amino acid numbers 374+((n−2)−x), in which x varies from 0 to n−2.

For example, for peptides of 10 amino acids (such that n=10), the starting position could be as “early” in the sequence as amino acid number 365 if x=n−2=8 (ie 365=373-8), such that the peptide would end at amino acid number 374 (374+(8−8=0)). On the other hand, the peptide could start at amino acid number 373 if x=0 (ie 373=373-0), and could end at amino acid 382 (374+(8−0=8)).

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: VKGI, KGIF, or LVKG. All peptides feature KG as a portion thereof. Peptides of from about five to about nine amino acids could optionally be similarly constructed.

(SEQ ID NO 390)

Variant III bridge:
PRHFRPKGAGSTFFVKIVLKEKHKK//EDKADPGHSEISSTRCPKAPGRV
LVHTSVSPSPDNLRRFALEHEA

This bridge is present between amino acid positions 250 (lys) and 251 (glu), and preferably comprises a peptide having a sequence taken from either side of these positions. For example, the peptide could optionally comprise a bridge portion of SEQ ID NO: 390, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KE, having a structure as follows (numbering according to SEQ ID NO:390): a sequence starting from any of amino acid number 250−x to 250; and ending at any of amino acid numbers 251+((n−2)−x), in which x varies from 0 to n−2.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: KKED, HKKE, or KEDK. All peptides feature KE as a portion thereof. Peptides of from about five to about nine amino acids could optionally be similarly constructed.

(SEQ ID NO 391)

Variant VII bridge:
PDMIFCLFHGKRYSPGESWIIPYLEPQGLMYCLRCTCSE //
NLTLPLDSGPHQSPASTTGPCLFHGKRYSPGESWH

This bridge is present between amino acid positions 45 (glu) and 46 (asn), and preferably comprises a peptide having a sequence taken from either side of these positions. For example, the peptide could optionally comprise a bridge portion of SEQ ID NO: 391, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EN, having a structure as follows (numbering according to SEQ ID NO:391): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: SENL, ENLT, or CSEN. All peptides feature EN as a portion thereof. Peptides of from about five to about nine amino acids could optionally be similarly constructed.

This variant also has a new N-terminal sequence, which may optionally be constructed as part of a bridge as described above: MALVGLPG.

(SEQ ID NO 392)

Variant VIII bridge:
TPSGLRAPPKSCQHNGTMYQHGEIFSAHELFPSRLPNQCVLCSCT //
MRQVSNRMKRTVCSRSMG

This bridge is present between amino acid positions 124 (thr) and 125 (met), and preferably comprises a peptide having a sequence taken from either side of these positions. For example, the peptide could optionally comprise a bridge portion of SEQ ID NO: 392, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TM, having a structure as follows (numbering according to SEQ ID NO:392): a sequence starting from any of amino acid number 124−x to 124 and ending at any of amino acid numbers 125+((n−2)−x), in which x varies from 0 to n−2, wherein the ending position is not greater than 142.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CTMR, SCTM, or TMRQ. All peptides feature TM as a portion thereof. Peptides of from about five to about nine amino acids could optionally be similarly constructed.

This variant also has a new N-terminal sequence, which may optionally be constructed as part of a bridge as described above:

MALVGLPG

(SEQ ID NO 393)

Variant IX bridge:
PDMFCLFHGKRYSPGESWHPYLEPQGLMYCLRCTCSE // NLTLPLDSG
PHQSPASTTGPCLFHGKRYSPGESWHPYLEPQGLMYCLRCTCS

This bridge is present between amino acid positions 45 (glu) and 46 (asn), and preferably comprises a peptide having a sequence taken from either side of these positions. For example, the peptide could optionally comprise a bridge portion of SEQ ID NO: 393, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EN, having a structure as follows (numbering according to SEQ ID NO:393): a sequence starting from any of amino acid number 45-x to 45; and ending at any of amino acid numbers 46+((n−2)−x), in which x varies from 0 to n−2; wherein if the peptide is 50 amino acids in length, the starting position cannot be any smaller than 1.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: SENL, ENLT, or CSEN. All peptides feature EN as a portion thereof. Peptides of from about five to about nine amino acids could optionally be similarly constructed.

This variant also has a new N-terminal sequence, which may optionally be constructed as part of a bridge as described above:
MALVGLPG

“Unique sequence”—as a result of alternative splicing, a non terminal exon is skipped (see for example variant 1 (exon 9b skipped), 2 (exons 9b and 3 are skipped), etc. Skipping of a non-terminal exon creates a unique sequence not present in the parent CHL2 which is the result of a ligation of the two exons flanking the “skipped” exon. This unique sequence results from the unique skipping pattern of the specific variant distinguishing the variant CHL2 of the invention from the parent chordin, or other known variants of chordin. Another possible unique sequence is intron-included sequences marked as exon 2a (variants IV, V, VI, VII, VIII) or exon 4a (variant X). Specific positions of the unique sequences are specified herein.

In order to understand the invention and to see how it may be carried out in practice, a preferred embodiment will now be described, by way of non-limiting example only, with reference to the accompanying drawings, described hereinbelow.

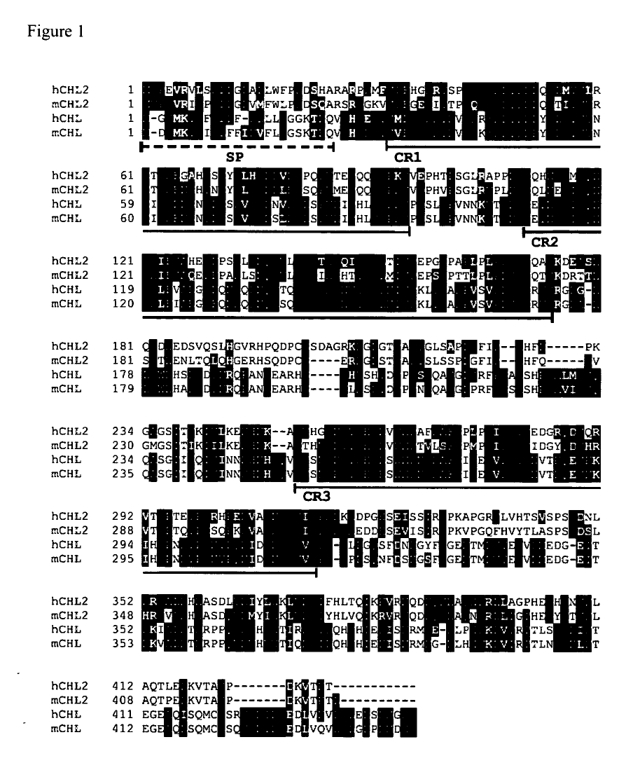

FIG. 1 shows a comparison of the human and mouse CHL2 variant I and CHL proteins. Amino acid sequence alignment of the orthologous and paralogous proteins indicates high conservation between these two vertebrate genes. The position of the signal peptide (SP) and the three CR repeats (CR1-CR3) is indicated. Sequences were aligned using the ClustalW program. Identical and similar residues are indicated by dark and light shading, respectively. Dashes indicate gaps introduced to align sequences. Protein sequences taken for the analysis were: hCHL2 (SEQ ID NO:11), mCHL2 (SEQ ID NO:96), hCHL (amino acid sequence corresponding to nucleotide sequence given in Genbank accession number AX175130), and mCHL (genebank accession number BC066832).

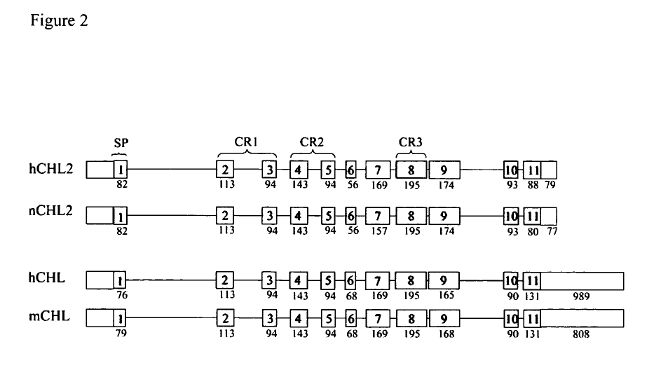

FIG. 2 shows a schematic representation of the human and mouse CHL2 and CHL genes (sequence identification numbers as for FIG. 1). Shown is the intron-exon genomic organization of the genes. Exons are depicted as boxes, and their size is given in bp. Introns, not drawn to scale, are drawn as thin lines. Coding and untranslated sequences are shown in gray and white, respectively. Sequences encoding for the signal peptide and the CR repeats are indicated on top. Note that CR1 and CR2 are each encoded by two exons, while CR3 is encoded by a single exon.

FIG. 3 shows alternative splicing of the hCHL2 gene. The exon-intron organization and the primers employed in the RT-PCR analysis are indicated on the top diagram, which shows the entire gene. The various splice variants identified are shown. UTRs are depicted in white, and the ORFs of the splice variants encoding different isoforms are indicated in gray or varying patterns. The size of the protein isoforms is given in amino acids, and the existence of a signal peptide (SP) and the CR repeats is indicated for each isoform.

Primers p1 (SEQ ID NO:399)+p4 (SEQ ID NO: 402) were used to detect variants I, II, III; primers p1 (SEQ ID NO:399)+p8 (SEQ ID NO: 406) were used to detect variants I, II; primers p2 (SEQ ID NO: 400)+p4 (SEQ ID NO: 402) were used to detect variants IV, V, VI, VII, VIII, IX; primers p3 (SEQ ID NO: 401)+p4 (SEQ ID NO: 402) were used to detect variant X; primers p2 (SEQ ID NO: 400)+p7 (SEQ ID NO: 405) were used to detect variants IV, VIII; primers p5 (SEQ ID NO: 403)+p7 (SEQ ID NO: 405) were used to detect variants containing exon 8; primers p1 (SEQ ID NO:399)+p6 (SEQ ID NO: 404) were used to detect variant III) in adult human tissues (results not shown).

The following describes the exons that characterize variants according to the present invention and primers that may optionally used to amplify each exon: exon 1 (p1 (SEQ ID NO:399)+p4 (SEQ ID NO: 402)) characterizes variants I, II and III; exon 2a (p2 (SEQ ID NO: 400)+p4 (SEQ ID NO: 402)) characterizes variants IV, V, VI, VII, VIII, IX; exon 4a (p3 (SEQ ID NO: 401)+p7 (SEQ ID NO: 405)) characterizes variant X; exon 8 (p5 (SEQ ID NO: 403)+p7 (SEQ ID NO: 405)) characterizes variants I, II, IV, VII, VIII, IX, X) Splice variants.

Relative expression of hCHL2 transcripts containing the amplicon of the unique exon 2a, SEQ ID NO: 442 (e.g., variant no. IV, V, VI, VII, VIII, IX), in normal and cancerous breast tissues was determined by real time PCR using primers for SEQ ID NO: 442 (SEQ ID NO: 440, 441). Expression was normalized to the averaged expression of four housekeeping genes PBGD (GenBank Accession No. BC019323; amplicon—SEQ ID NO: 433, primers SEQ ID Nos: 431, 432), HPRT1 (GenBank Accession No. NM_{—}000194; amplicon—SEQ ID NO: 430, primers SEQ ID Nos: 428, 429), G-6_PD (GenBank Accession No. NM_{—}000402; amplicon—SEQ ID NO: 439, primers SEQ ID Nos: 437, 438) and SDHA (GenBank Accession No. NM_{—}004168; amplicon—SEQ ID NO: 436, primers SEQ ID Nos: 434, 435); results not shown. However, the primers were able to successfully amplify the desired amplicon.

Relative expression of hCHL2 transcripts containing the amplicon of the unique exon 4a, SEQ ID NO: 448, (e.g., variant no. X) in normal, benign and cancerous prostate tissues was determined by real time PCR using primers for SEQ ID NO: 448 (SEQ ID NO: 446, 447). Expression was normalized to the averaged expression of four housekeeping genes; results not shown. However, the primers were able to successfully amplify the desired amplicon.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated polynucleotide comprising a nucleic acid sequence according to SEQ ID NO:1.

2. The isolated polynucleotide of claim 1, comprising a polynucleotide having a nucleic acid sequence according to any one of SEQ ID NOs:2-7.

3. An isolated polypeptide comprising an amino acid sequence according to SEQ ID NO:9.

4. An isolated chimeric polypeptide encoding for S71513_P2 (SEQ ID NO:9), comprising a first amino acid sequence being at least 90% homologous to MKVSAALLCLLLIAATFIPQGLAQPDAINAPVTCCYNFTNRKISVQRLASYRRITSSKCP KEAV corresponding to amino acids 1-64 of SY02_HUMAN, which also corresponds to amino acids 1-64 of S71513_P2 (SEQ ID NO:9), and a second amino acid sequence comprising a polypeptide having the sequence M corresponding to amino acid 65 of S71513_P2 (SEQ ID NO:9), wherein said first amino acid sequence and second amino acid sequence are contiguous and in a sequential order.

5. An antibody capable of specifically binding to an epitope of an amino acid sequence of claim 3.

6. The antibody of claim 5, wherein said amino acid sequence corresponds to a bridge including amino acids 64 and 65 of SEQ ID NO: 9, of at least about 10 amino acids (amino acids 55-65 of SEQ ID NO:9), at least about 20 amino acids (amino acids 45-65 of SEQ ID NO:9), at least about 30 amino acids (amino acids 35-65 of SEQ ID NO:9) and at least about 40 amino acids (amino acids 25-65 of SEQ ID NO:9) in length.

7. The antibody of claim 5, wherein said antibody is capable of differentiating between a splice variant having said epitope and a corresponding known protein, SY02_HUMAN.

8. A kit for detecting endometriosis, comprising a kit detecting overexpression of a splice variant of claim 1.

9. The kit of claim 8, wherein said kit comprises a NAT-based technology.

10. The kit of claim 9, wherein, where a nucleic acid sequence is utilized, said kit further comprises at least one primer pair capable of selectively hybridizing to the nucleic acid sequence.

11. The kit of claim 10, wherein, where a nucleic acid sequence is utilized, said kit further comprises at least one oligonucleotide capable of selectively hybridizing to the nucleic acid sequence.

12. A kit for detecting endometriosis, comprising a kit comprising an antibody according to claim 5.

13. The kit of claim 12, wherein said kit further comprises at least one reagent for performing an ELISA or a Western blot.

14. A method for detecting endometriosis, comprising detecting overexpression and/or underexpression of a splice variant according to claim 1.

15. The method of claim 14, wherein said detecting overexpression is performed with a NAT-based technology.

16. A method for detecting endometriosis, comprising detecting overexpression and/or underexpression of a splice variant according to claim 3, wherein said detecting overexpression is performed with an immunoassay.

17. A method for detecting endometriosis, comprising detecting overexpression and/or underexpression of a splice variant performed with an immunoassay, according to claim 5.

18. A biomarker capable of detecting endometriosis, comprising a nucleic acid sequence or a fragment thereof according to claim 1, or an amino acid sequence or a fragment thereof according to claim 3.

19. A method for screening for endometriosis, comprising detecting endometriosis cells with a biomarker according to claim 18.

20. A method for diagnosing endometriosis, comprising detecting endometriosis cells with a biomarker according to claim 18.

21. A method for monitoring disease progression and/or treatment efficacy and/or relapse of endometriosis, comprising detecting endometriosis cells with a biomarker according to claim 18.

22. A method of selecting a therapy for endometriosis, comprising detecting endometriosis cells with a biomarker according to claim 18 and selecting a therapy according to said detection.
